(19) United States
(12) Patent Application Publication
(10) Pub. No.: US 2023/0165142 A1
(43) Pub. Date: May 25, 2023

(54) ORGANIC ELECTROLUMINESCENT COMPOUND, A PLURALITY OF HOST MATERIALS COMPRISING THE SAME, AND ORGANIC ELECTROLUMINESCENT DEVICE

(71) Applicant: ROHM AND HAAS ELECTRONIC MATERIALS KOREA LTD., Chungcheongnam-do (KR)

(72) Inventors: Doo-Hyeon MOON, Gyeonggi-do (KR); Du-Yong Park, Gyeonggi-do (KR); Su-Hyun Lee, Gyeonggi-do (KR); Sang-Hee Cho, Gyeonggi-do (KR); Ji-Song Jun, Gyeonggi-do (KR); So-Young Jung, Gyeonggi-do (KR); Hyo-Nim Shin, Gyeonggi-do (KR)

(21) Appl. No.: 17/979,252

(22) Filed: Nov. 2, 2022

(30) Foreign Application Priority Data
Nov. 5, 2021 (KR) 10-2021-0151027
Apr. 6, 2022 (KR) 10-2022-0042712
Sep. 1, 2022 (KR) 10-2022-0110701

(51) Int. Cl. H10K 85/60; C07D 421/04; C07D 421/14; C07F 7/08; H10K 85/40; C07F 7/30; H10K 85/30; C07D 421/10; C07D 491/048; H10K 50/11

(52) U.S. Cl. CPC H10K 85/657; H10K 85/654; C07D 421/04; H10K 85/6574; C07D 421/14; H10K 85/6576; H10K 85/6572; C07F 7/0812; H10K 85/40; C07F 7/30; H10K 85/30; H10K 85/652; C07D 421/10; C07D 491/048; H10K 85/636; H10K 85/631; H10K 85/633; H10K 85/615; H10K 85/622; H10K 50/11; C07B 2200/05

(57) ABSTRACT

The present disclosure relates to an organic electroluminescent compound, a plurality of host materials comprising the same, and an organic electroluminescent device. By comprising an organic electroluminescent compound according to the present disclosure and a plurality of host materials comprising the same, an organic electroluminescent device having a low driving voltage and/or a high luminous efficiency and/or a long lifespan can be prepared.

### TECHNICAL FIELD

The present disclosure relates to an organic electroluminescent compound, a plurality of host materials comprising the same, and an organic electroluminescent device.

### BACKGROUND ART

An electroluminescent device (EL device) is a self light-emitting display device which has advantages in that it provides a wider viewing angle, a greater contrast ratio, and a faster response time. An organic electroluminescent device (OLED) was first developed by Eastman Kodak in 1987, by using small aromatic diamine molecules and aluminum complexes as materials for forming a light-emitting layer [Appl. Phys. Lett. 51, 913, 1987].

The light-emitting material of an OLED is the most important factor determining luminous efficiency of the device, and may be classified into a host material and a dopant material in a functional aspect. A light-emitting material can be used by mixing a host and a dopant in order to improve color purity, luminous efficiency, and stability. Generally, a device having excellent electroluminescent (EL) characteristics has a structure comprising a light-emitting layer formed by doping a dopant to a host. When using such a dopant/host material system as a light-emitting material, their selection is important since host materials greatly influence the efficiency and lifespan of the light-emitting device.

Recently, an urgent task is the development of an OLED having high efficiency and long lifespan characteristics. In particular, the development of highly excellent light-emitting material over conventional light-emitting materials is urgently required, considering the EL properties necessary for medium and large-sized OLED panels.

Korean Patent Application Laid-Open No. 10-2017-0115940 discloses an organic electroluminescent device including a naphthooxazole-based or naphthothiazole-based compound, however, there is a continuous need to develop a light-emitting material having more improved performance, e.g., improved driving voltage, luminous efficiency, and/or lifespan characteristics.

### DISCLOSURE OF THE INVENTION

### Technical Problem

The object of the present disclosure is firstly, to provide an organic electroluminescent compound and a plurality of host materials comprising the same which are able to produce an organic electroluminescent device having a low driving voltage and/or high luminous efficiency and/or long lifespan characteristics, and secondly, to provide an organic electroluminescent device comprising the organic electroluminescent compound and/or the plurality of host materials.

### Solution to Problems

As a result of intensive studies to solve the technical problem above, the present inventors found that the aforementioned objective can be achieved by an organic electroluminescent compound represented by the following formula 1, so that the present invention was completed.

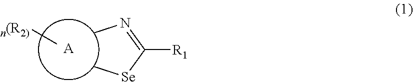

in formula 1,

ring A is a naphthalene ring;

R₁ represents a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, -L′₁-SiR′₁R′₂R′₃, -L′₁-GeR′₁R′₂R′₃, or -L′₂-NR′₄R′₅;

R₂ represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, -L′1-SiR′1R′₂R′₃, or -L′₂-NR′₄R′₅; or may be linked to the adjacent substituents to form a ring(s);

L′₁ and L′₂ each independently represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

R′₁ to R′₅ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and

n is an integer of 0 to 6.

### Advantageous Effects of Invention

By comprising an organic electroluminescent compound according to the present disclosure, an organic electroluminescent device having a low driving voltage and/or high luminous efficiency and/or long lifespan characteristics can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to an organic electroluminescent compound represented by formula 1, an organic electroluminescent material comprising the same, and an organic electroluminescent device comprising the organic electroluminescent compound and/or the organic electroluminescent material.

The present disclosure relates to a plurality of host materials comprising a first host material represented by formula 1 and a second host material which is different from the first host material, and an organic electroluminescent device comprising the same.

The term “organic electroluminescent compound” in the present disclosure means a compound that may be used in an organic electroluminescent device, and may be comprised in any material layer constituting an organic electroluminescent device, as necessary.

Herein, the term “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such at least two compounds may be comprised in the same layer or different layers, and may be mixture-evaporated or co-evaporated, or may be individually evaporated.

Herein, “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, two or more compounds may be mixture-evaporated to form a layer or may be individually and simultaneously co-evaporated to form a layer.

Herein, “(C1-C30)alkyl(ene)” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the term “(C3-C30)cycloalkyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, “(C6-C30)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3- to 30-membered)heteroaryl(ene)” is an aryl having 3 to 30 ring backbone atoms including at least one, preferably 1 to 4 heteroatoms selected from the group consisting of B, N, O, S, Si, P, Se, and Ge, in which the number of the ring backbone carbon atoms is preferably 5 to 25. The above heteroaryl(ene) may be a monocyclic ring, or a fused ring condensed with at least one benzene ring; and may be partially saturated. Also, the above heteroaryl or heteroarylene herein may be one formed by linking at least one heteroaryl or aryl group to a heteroaryl group via a single bond(s). Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthiridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthrdinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrmidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Herein, the term “a fused ring of (C3-C30) aliphatic ring and (C6-C30) aromatic ring” means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C3-C30) aliphatic ring and (C6-C30) aromatic ring may be replaced with at least one heteroatom selected from B, N, O, S, Si and P, preferably at least one heteroatom selected from N, O and S. The term “Halogen” in the present disclosure includes F, Cl, Br, and I.

In addition, “ortho (o),” “meta (m),” and “para (p)” are meant to signify the substitution position of all substituents. Ortho position is a compound with substituents, which are adjacent to each other, e.g., at the 1 and 2 positions on benzene. Meta position is the next substitution position of the immediately adjacent substitution position, e.g., a compound with substituents at the 1 and 3 positions on benzene. Para position is the next substitution position of the meta position, e.g., a compound with substituents at the 1 and 4 positions on benzene.

Herein, “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si and P, preferably, N. O and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” means that a hydrogen atom in a certain functional group is replaced with another atom or functional group, i.e., a substituent, and substituted with a group to which two or more substituents are connected among the substituents. For example, “a substituent to which two or more substituents are connected” may be pyridine-triazine. That is, pyridine-triazine may be heteroaryl or may be interpreted as one substituent in which two heteroaryls are connected. The substituents of the substituted alkyl(ene), the substituted alkenyl, the substituted aryl(ene), the substituted heteroaryl(ene), the substituted cycloalkyl(ene), and the substituted alkoxy in the formulas of the present disclosure each independently represent, preferably, at least one selected from the group consisting of deuterium; halogen; cyano; carboxyl; nitro; hydroxy; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted with at least one (C6-C30)aryl; (C6-C30)aryl unsubstituted or substituted with at least one of (C1-C30)alkyl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; tri(C6-C30)arylgermanyl; amino; mono- or di-(C1-C30)alkylamino; mono- or di-(C2-C30)alkenylamino; mono- or di-(C6-C30)arylamino unsubstituted or substituted with (C1-C30)alkyl; mono- or di-(3- to 30-membered)heteroarylamino; (C1-C30)alkyl(C2-C30)alkenylamino; (C1-C30)alkyl(C6-C30)arylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl, etc.

In the formula herein, when there are a plurality of substituents represented by the same symbol, each of the substituents represented by the same symbol may be the same or different.

Hereinafter, an organic electroluminescent compound according to one embodiment will be described.

The organic electroluminescent compound according to one embodiment is represented by the following formula 1.

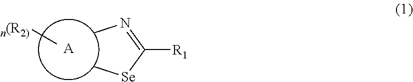

in formula 1,

ring A is a naphthalene ring;

R₁ represents a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, -L′₁-SiR′₁R′₂R′₃, -L′₁-GeR′₁R′₂R′₃, or -L′₂-NR′₄R′₅;

R₂ represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, -L′₁-SiR′₁R′₂R′₃, or -L′₂-NR′₄R′₅; or may be linked to the adjacent substituents to form a ring(s);

L′₁ and L′₂ each independently represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

R′₁ to R′₅ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and

n is an integer of 0 to 6.

According to one embodiment, ring A is a naphthalene ring, and the compound represented by formula 1 may be represented by any one of the following formulas 1-1 to 1-3.

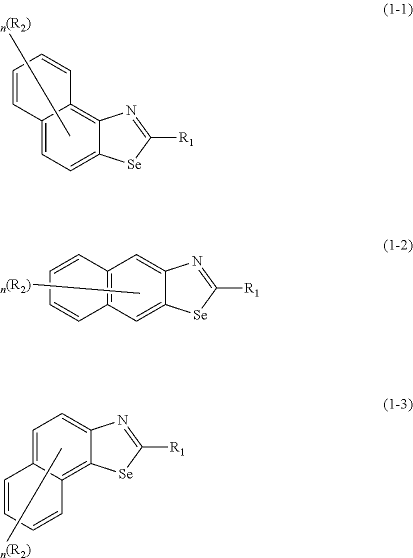

in formulas 1-1 to 1-3,

R₁, R₂, and n are as defined in formula 1.

According to one embodiment, the compound represented by formula 1 may be represented by any one of the following formulas 1-4 to 1-6.

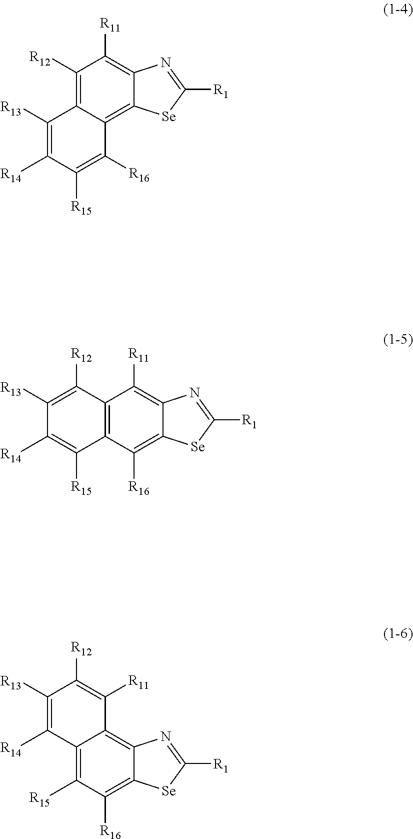

in formulas 1-4 to 1-6,

R₁ is as defined in formula 1;

R₁₁ to R₁₆ are each independently as defined as R₂ in formula 1; provided that at least one of R₁ and R₁₁ to R₁₆ represent(s) -L₁-HAr;

L₁ represents a single bond, a substituted or unsubstituted (C6-C30)arylene, a substituted or unsubstituted (3- to 30-membered)heteroarylene, or a substituted or unsubstituted (C3-C30)cycloalkylene; and

HAr represents a substituted or unsubstituted nitrogen-containing (3- to 30-membered)heteroaryl.

In one embodiment, R₁ may be a substituted or unsubstituted (C6-C30)aryl, a substituted or unsubstituted (5- to 30-membered)heteroaryl, -L′₁-SiR′₁R′₂R′₃, or -L′₁-GeR′₁R′₂R′₃, preferably a substituted or unsubstituted (C6-C25)aryl, a substituted or unsubstituted (5- to 25-membered)heteroaryl, -L′₁-SiR′₁R′₂R′₃, or -L′₁-GeR′₁R′₂R′₃, more preferably a substituted or unsubstituted (C6-C18)aryl, a substituted or unsubstituted (5- to 18-membered)heteroaryl, -L′₁-SiR′₁R′₂R′₃, or -L′₁-GeR′₁R′₂R′₃. Wherein, L′₁ may be a substituted or unsubstituted (C6-C30)arylene, and R′₁ to R′₃ each independently may be a substituted or unsubstituted (C6-C30)aryl. For example, R₁ may be a substituted or unsubstituted phenyl, a substituted or unsubstituted biphenyl, a substituted or unsubstituted naphthyl, a substituted or unsubstituted phenanthrenyl, a substituted or unsubstituted fluorenyl, a substituted or unsubstituted carbazolyl, a substituted or unsubstituted dibenzofuranyl, a substituted or unsubstituted dibenzothiophenyl, phenyl substituted with triphenylsilanyl, or phenyl substituted with triphenylgermanyl. For example, the substituent of the substituents may be at least one selected from deuterium, cyano, methyl, and phenyl.

In one embodiment, R₁₁ to R₁₆ each independently represent hydrogen, deuteriumcyano, or -L₁-HAr.

In one embodiment, L₁ may be a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 25-membered)heteroarylene, more preferably a single bond, a substituted or unsubstituted (C6-C18)arylene, or a substituted or unsubstituted (5- to 18-membered)heteroarylene. For example, L₁ may be a single bond, a substituted or unsubstituted phenylene, a substituted or unsubstituted m-biphenylene, a substituted or unsubstituted o-biphenylene, a substituted or unsubstituted naphthalenylene, a substituted or unsubstituted fluorenylene, a substituted or unsubstituted pyridinylene, a substituted or unsubstituted phenylpyridinylene, a substituted or unsubstituted carbazolylene, a substituted or unsubstituted dibenzofuranylene, or a substituted or unsubstituted dibenzothiophenylene. For example, the substituent of the substituents may be at least one selected from deuterium, methyl, and phenyl.

In one embodiment, HAr may be a substituted or unsubstituted nitrogen containing (5- to 30-membered)heteroaryl. For example, HAr may be a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted benzoxazolyl, a substituted or unsubstituted pyrazolyl, a substituted or unsubstituted naphthyridinyl, or a substituted or unsubstituted benzothienopyrimidinyl, for example, a substituted or unsubstituted triazinyl, a substituted or unsubstituted pyrimidinyl, a substituted or unsubstituted quinazolinyl, a substituted or unsubstituted quinoxalinyl, a substituted or unsubstituted benzoxazolyl, or a substituted or unsubstituted benzothienopyrimidinyl. The substituent of the substituents may be a substituted or unsubstituted (C6-C30)aryl or a substituted or unsubstituted (5- to 30-membered)heteroaryl, for example, may be at least one of phenyl unsubstituted or substituted with at least one selected from deuterium; cyano; methyl; naphthyl; triphenylsilanyl; and triphenylgermanyl, p-biphenyl, m-biphenyl, o-biphenyl, p-terphenyl, m-terphenyl, naphthyl unsubstituted or substituted with phenyl, phenanthrenyl, chrysenyl, fluorenyl unsubstituted or substituted with at least one of methyl and phenyl, spirobifluorenyl, carbazolyl unsubstituted or substituted with at least one of phenyl and biphenyl, dibenzofuranyl, dibenzothiophenyl, and dibenzoselenophenyl.

In one embodiment, -L₁-HAr may be represented by the following formula 1′.

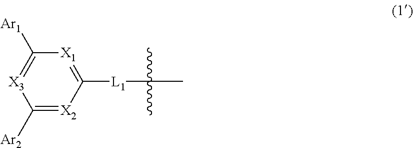

in formula 1′,

X₁ to X₃ each independently represent N or CH; provided that, at least one of X₁ to X₃ represent(s) N;

L₁ is as defined as L₁ in formulas 1-4 to 1-6; and

Ar₁ and Ar₂ each independently represent a substituted or unsubstituted (C6-C30)aryl or a substituted or unsubstituted (3- to 30-membered)heteroaryl.

In one embodiment, at least one of X₁ to X₃ represent(s) N, preferably at least two of X₁ to X₃ may be N, more preferably all of X₁ to X₃ may be N.

In one embodiment. Ar₁ and Ar₂ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C25)aryl or a substituted or unsubstituted (5- to 18-membered)heteroaryl. For example, Ar₁ and Ar₂ each independently may be a substituted or unsubstituted phenyl, a substituted or unsubstituted p-biphenyl, a substituted or unsubstituted m-biphenyl, a substituted or unsubstituted o-biphenyl, a substituted or unsubstituted p-terphenyl, a substituted or unsubstituted m-terphenyl, a substituted or unsubstituted naphthyl, a substituted or unsubstituted fluorenyl, a substituted or unsubstituted phenanthrenyl, a substituted or unsubstituted chrysenyl, a substituted or unsubstituted spirobifluorenyl, a substituted or unsubstituted carbazolyl, a substituted or unsubstituted dibenzofuranyl, a substituted or unsubstituted dibenzothiophenyl, a substituted or unsubstituted dibenzoselenophenyl. For example, the substituent of the substituents may be at least one of deuterium; cyano; methyl; phenyl; biphenyl; naphthyl; triphenylsilanyl; and triphenylgermanyl.

According to one embodiment, the organic electroluminescent compound of formula 1 may be more specifically illustrated by the following compounds, but is not limited thereto.

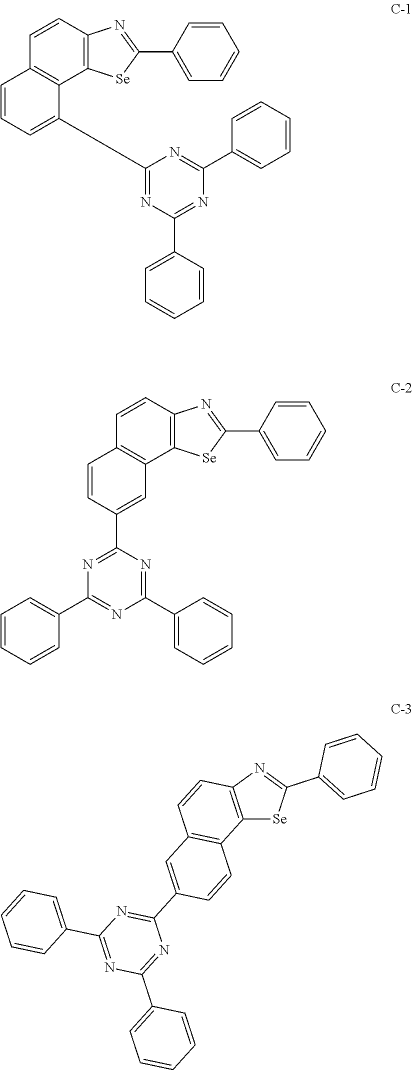 [SMILES: c1ccc(-c2nc(-c3ccccc3)nc(-c3cccc4ccc5nc(-c6ccccc6)[se]c5c34)n2)cc1 ; c1ccc(-c2nc(-c3ccccc3)nc(-c3ccc4ccc5nc(-c6ccccc6)[se]c5c4c3)n2)cc1 ; c1ccc(-c2nc(-c3ccccc3)nc(-c3ccc4c(ccc5nc(-c6ccccc6)[se]c54)c3)n2)cc1]

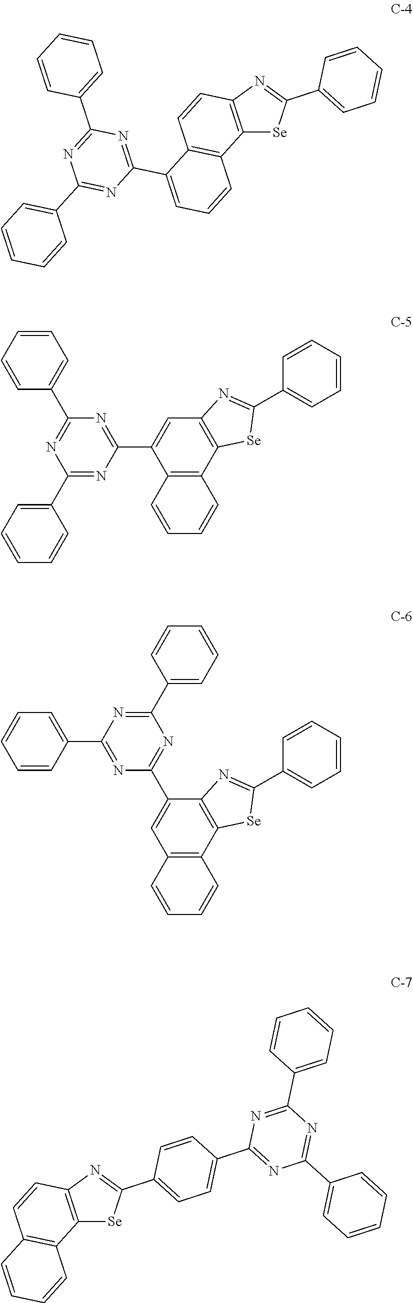 [SMILES: c1ccc(-c2nc(-c3ccccc3)nc(-c3cccc4c3ccc3nc(-c5ccccc5)[se]c34)n2)cc1 ; c1ccc(-c2nc(-c3ccccc3)nc(-c3cc4nc(-c5ccccc5)[se]c4c4ccccc34)n2)cc1 ; c1ccc(-c2nc(-c3ccccc3)nc(-c3cc4ccccc4c4[se]c(-c5ccccc5)nc34)n2)cc1 ; c1ccc(-c2nc(-c3ccccc3)nc(-c3ccc(-c4nc5ccc6ccccc6c5[se]4)cc3)n2)cc1]

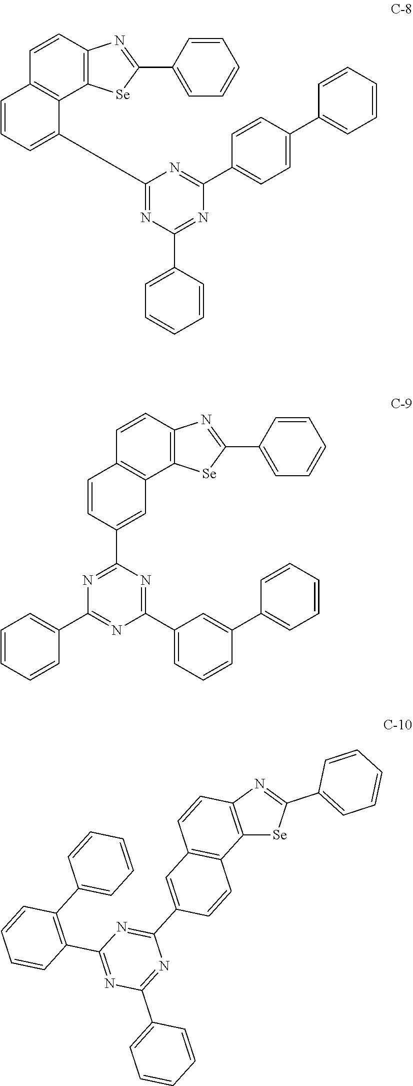 [SMILES: c1ccc(-c2ccc(-c3nc(-c4ccccc4)nc(-c4cccc5ccc6nc(-c7ccccc7)[se]c6c45)n3)cc2)cc1 ; c1ccc(-c2cccc(-c3nc(-c4ccccc4)nc(-c4ccc5ccc6nc(-c7ccccc7)[se]c6c5c4)n3)c2)cc1 ; c1ccc(-c2nc(-c3ccc4c(ccc5nc(-c6ccccc6)[se]c54)c3)nc(-c3ccccc3-c3ccccc3)n2)cc1]

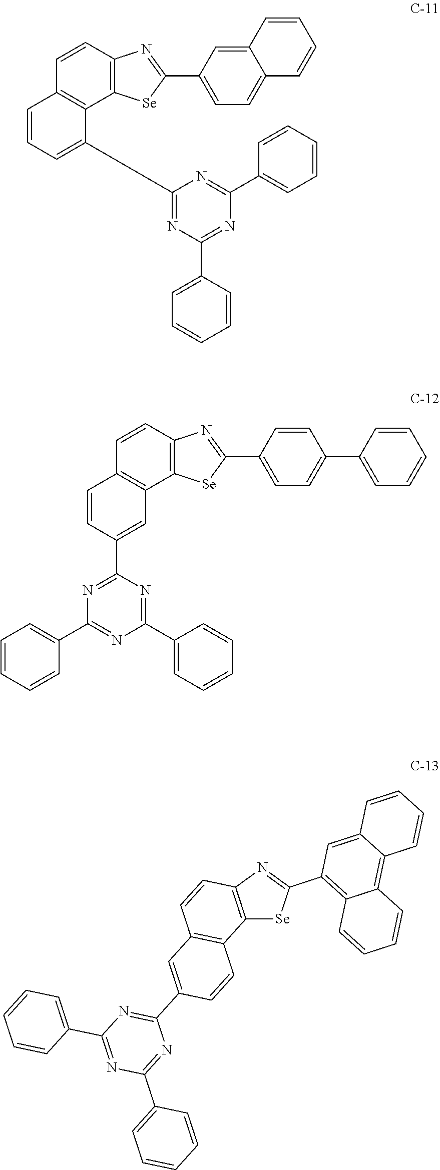 [SMILES: c1ccc(-c2nc(-c3ccccc3)nc(-c3cccc4ccc5nc(-c6ccc7ccccc7c6)[se]c5c34)n2)cc1 ; c1ccc(-c2ccc(-c3nc4ccc5ccc(-c6nc(-c7ccccc7)nc(-c7ccccc7)n6)cc5c4[se]3)cc2)cc1 ; c1ccc(-c2nc(-c3ccccc3)nc(-c3ccc4c(ccc5nc(-c6cc7ccccc7c7ccccc67)[se]c54)c3)n2)cc1]

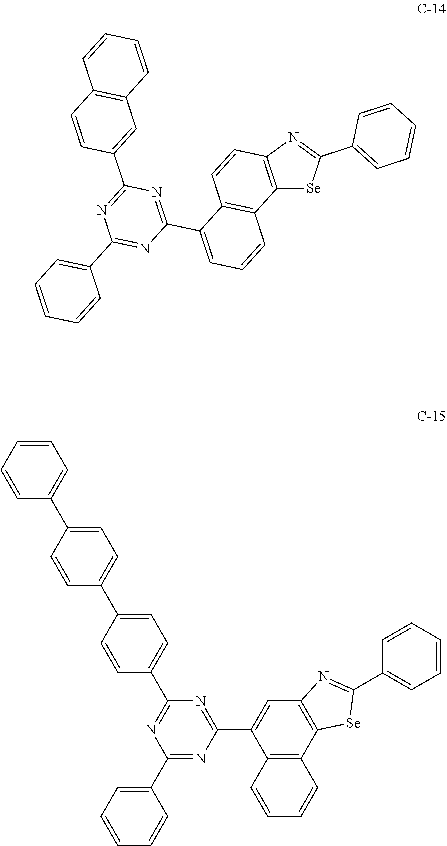 [SMILES: c1ccc(-c2nc(-c3ccc4ccccc4c3)nc(-c3cccc4c3ccc3nc(-c5ccccc5)[se]c34)n2)cc1 ; c1ccc(-c2ccc(-c3ccc(-c4nc(-c5ccccc5)nc(-c5cc6nc(-c7ccccc7)[se]c6c6ccccc56)n4)cc3)cc2)cc1]

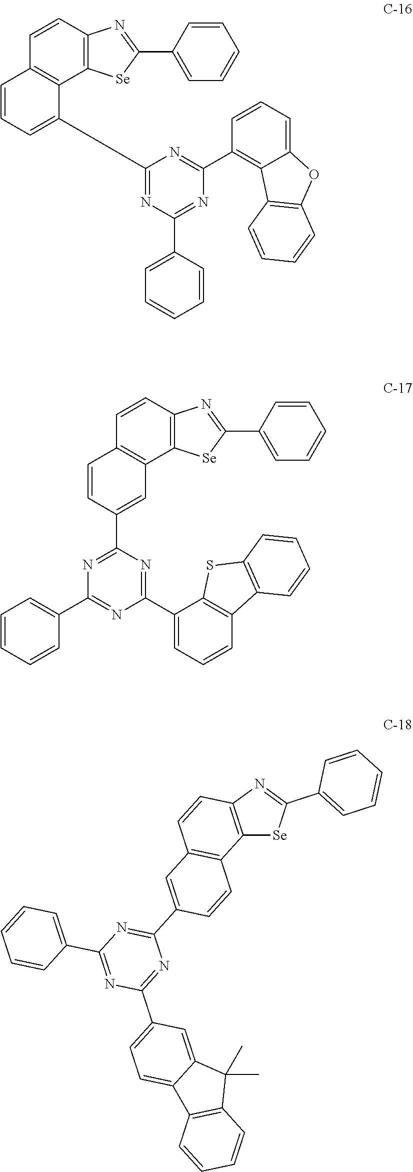 [SMILES: c1ccc(-c2nc(-c3cccc4oc5ccccc5c34)nc(-c3cccc4ccc5nc(-c6ccccc6)[se]c5c34)n2)cc1 ; c1ccc(-c2nc(-c3ccc4ccc5nc(-c6ccccc6)[se]c5c4c3)nc(-c3cccc4c3sc3ccccc34)n2)cc1 ; CC1(C)c2ccccc2-c2ccc(-c3nc(-c4ccccc4)nc(-c4ccc5c(ccc6nc(-c7ccccc7)[se]c65)c4)n3)cc21]

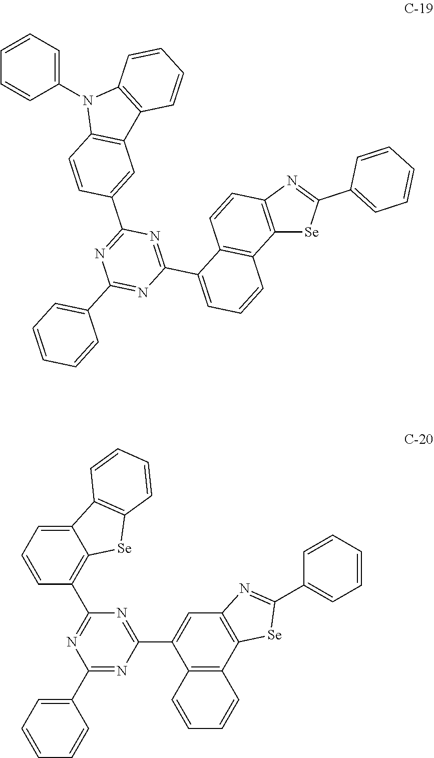 [SMILES: c1ccc(-c2nc(-c3ccc4c(c3)c3ccccc3n4-c3ccccc3)nc(-c3cccc4c3ccc3nc(-c5ccccc5)[se]c34)n2)cc1 ; c1ccc(-c2nc(-c3cc4nc(-c5ccccc5)[se]c4c4ccccc34)nc(-c3cccc4c3[se]c3ccccc34)n2)cc1]

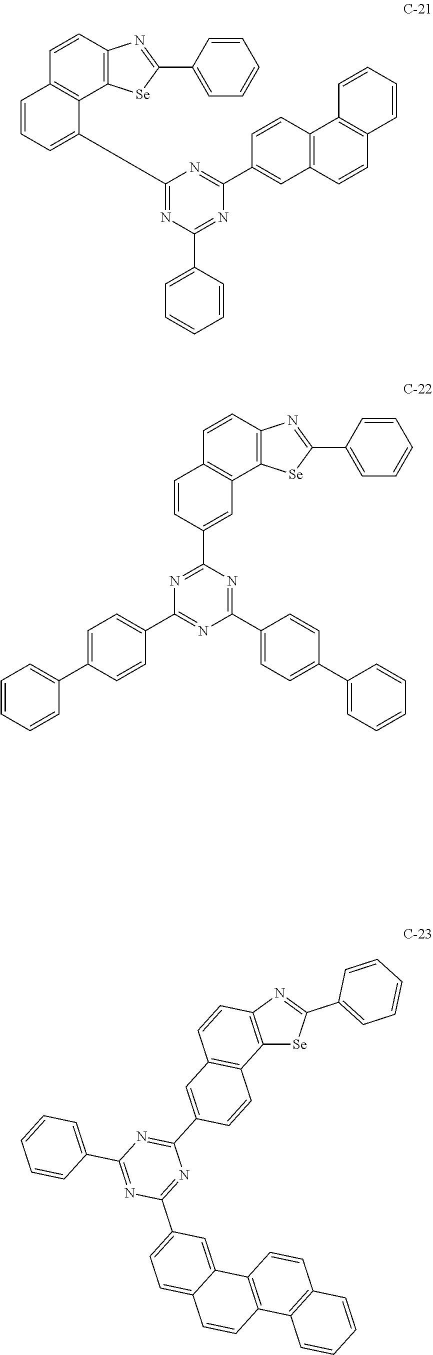 [SMILES: c1ccc(-c2nc(-c3ccc4c(ccc5ccccc54)c3)nc(-c3cccc4ccc5nc(-c6ccccc6)[se]c5c34)n2)cc1 ; c1ccc(-c2ccc(-c3nc(-c4ccc(-c5ccccc5)cc4)nc(-c4ccc5ccc6nc(-c7ccccc7)[se]c6c5c4)n3)cc2)cc1 ; c1ccc(-c2nc(-c3ccc4c(ccc5nc(-c6ccccc6)[se]c54)c3)nc(-c3ccc4ccc5c6ccccc6ccc5c4c3)n2)cc1]

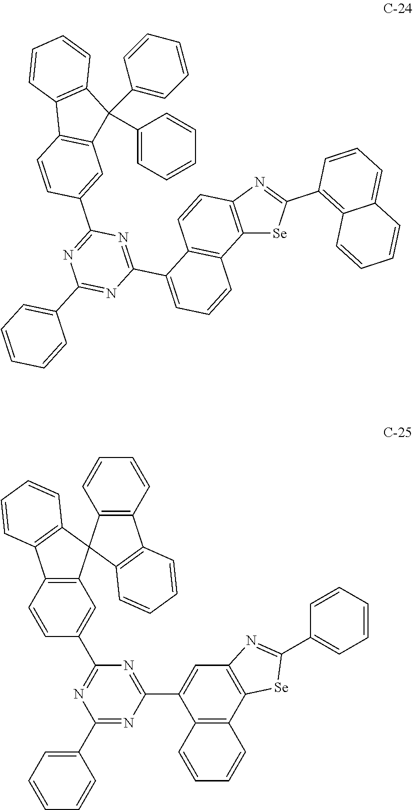 [SMILES: c1ccc(-c2nc(-c3ccc4c(c3)C(c3ccccc3)(c3ccccc3)c3ccccc3-4)nc(-c3cccc4c3ccc3nc(-c5cccc6ccccc56)[se]c34)n2)cc1 ; c1ccc(-c2nc(-c3ccc4c(c3)C3(c5ccccc5-c5ccccc53)c3ccccc3-4)nc(-c3cc4nc(-c5ccccc5)[se]c4c4ccccc34)n2)cc1]

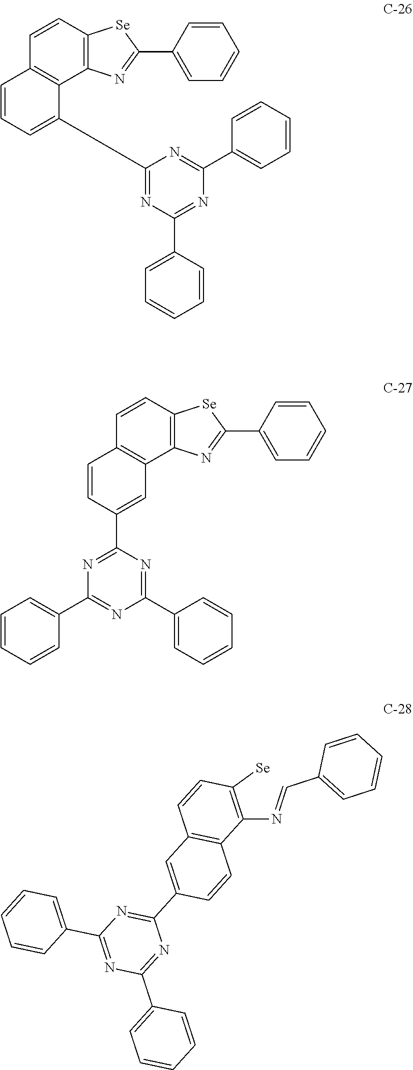

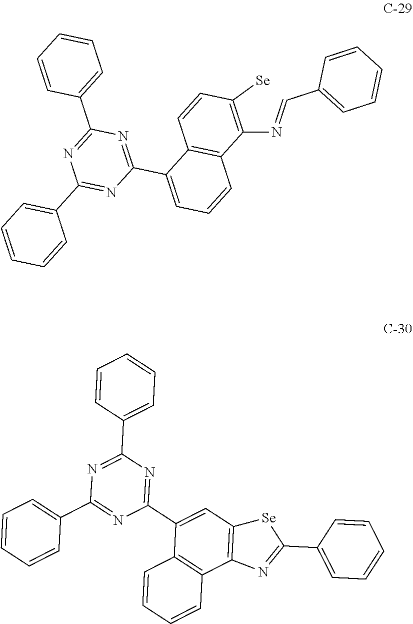

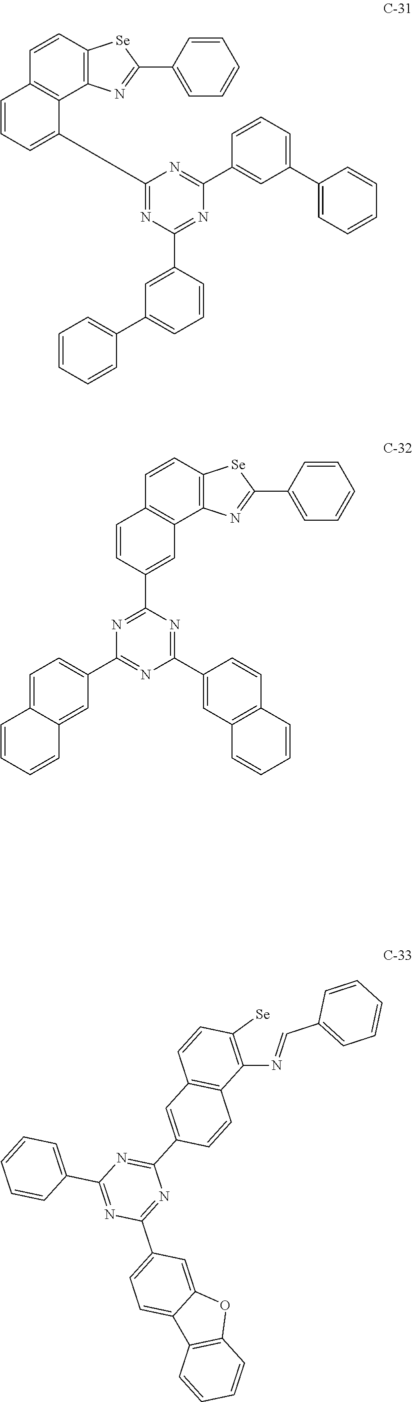

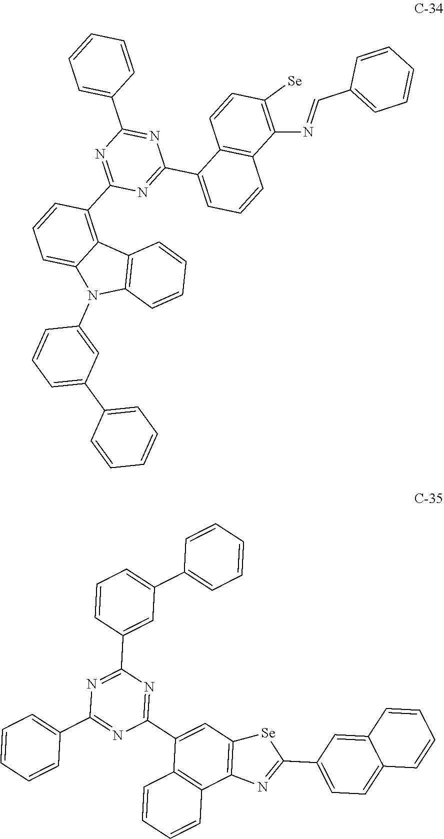

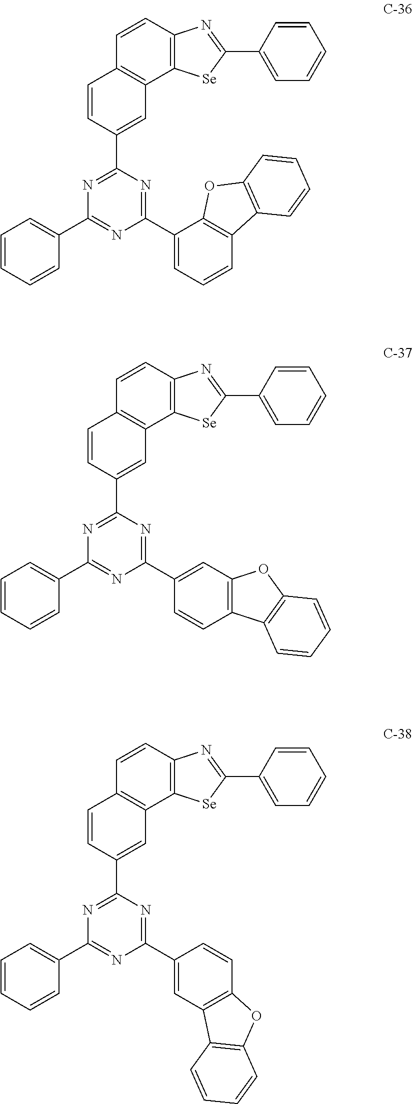 [SMILES: c1ccc(-c2nc(-c3ccc4ccc5nc(-c6ccccc6)[se]c5c4c3)nc(-c3cccc4c3oc3ccccc34)n2)cc1 ; c1ccc(-c2nc(-c3ccc4c(c3)oc3ccccc34)nc(-c3ccc4ccc5nc(-c6ccccc6)[se]c5c4c3)n2)cc1 ; c1ccc(-c2nc(-c3ccc4oc5ccccc5c4c3)nc(-c3ccc4ccc5nc(-c6ccccc6)[se]c5c4c3)n2)cc1]

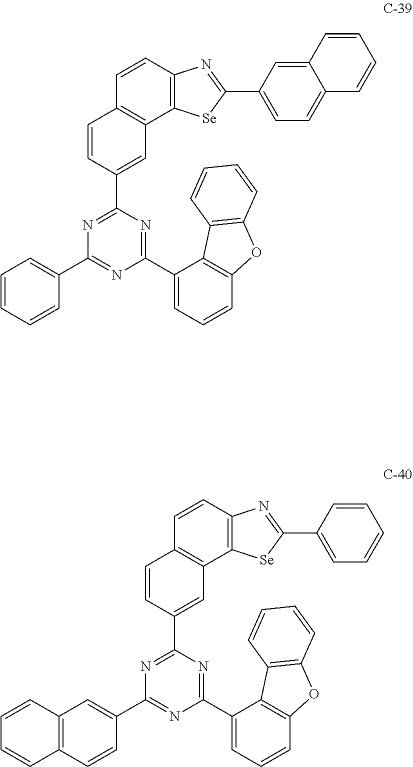 [SMILES: c1ccc(-c2nc(-c3ccc4ccc5nc(-c6ccc7ccccc7c6)[se]c5c4c3)nc(-c3cccc4oc5ccccc5c34)n2)cc1 ; c1ccc(-c2nc3ccc4ccc(-c5nc(-c6ccc7ccccc7c6)nc(-c6cccc7oc8ccccc8c67)n5)cc4c3[se]2)cc1]

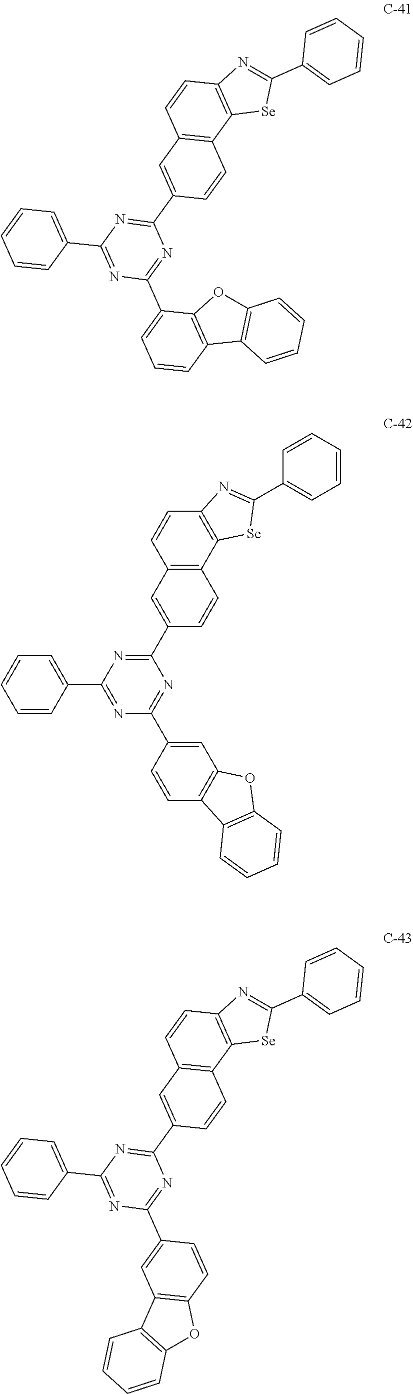 [SMILES: c1ccc(-c2nc(-c3ccc4c(ccc5nc(-c6ccccc6)[se]c54)c3)nc(-c3cccc4c3oc3ccccc34)n2)cc1 ; c1ccc(-c2nc(-c3ccc4c(ccc5nc(-c6ccccc6)[se]c54)c3)nc(-c3ccc4c(c3)oc3ccccc34)n2)cc1 ; c1ccc(-c2nc(-c3ccc4c(ccc5nc(-c6ccccc6)[se]c54)c3)nc(-c3ccc4oc5ccccc5c4c3)n2)cc1]

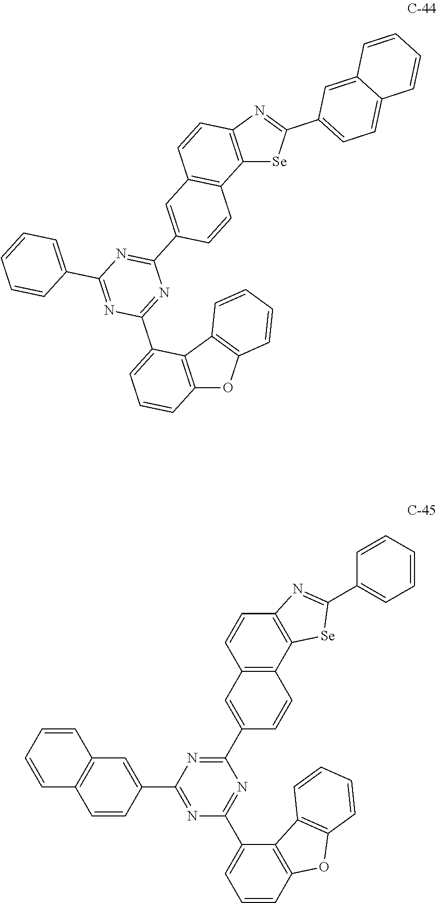 [SMILES: c1ccc(-c2nc(-c3ccc4c(ccc5nc(-c6ccc7ccccc7c6)[se]c54)c3)nc(-c3cccc4oc5ccccc5c34)n2)cc1 ; c1ccc(-c2nc3ccc4cc(-c5nc(-c6ccc7ccccc7c6)nc(-c6cccc7oc8ccccc8c67)n5)ccc4c3[se]2)cc1]

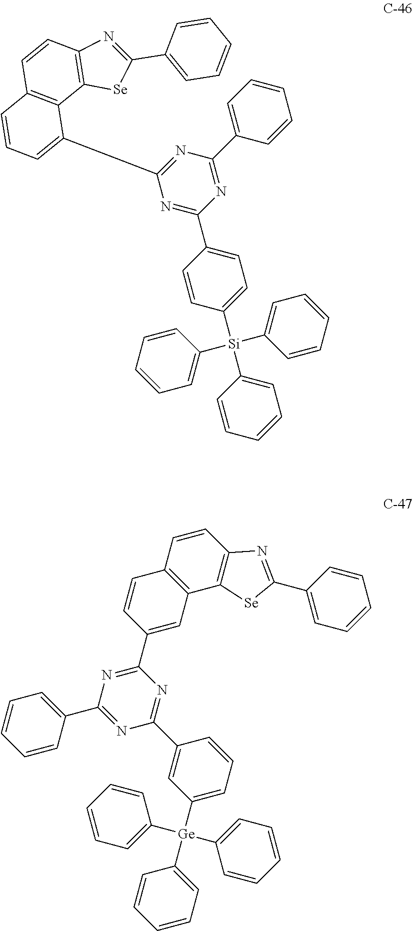

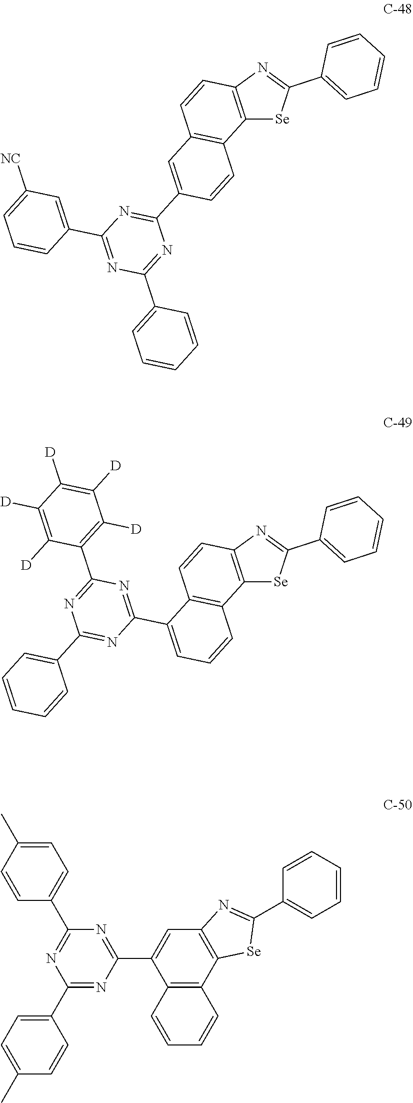 [SMILES: N#Cc1cccc(-c2nc(-c3ccccc3)nc(-c3ccc4c(ccc5nc(-c6ccccc6)[se]c54)c3)n2)c1 ; [2H]c1c([2H])c([2H])c(-c2nc(-c3ccccc3)nc(-c3cccc4c3ccc3nc(-c5ccccc5)[se]c34)n2)c([2H])c1[2H] ; Cc1ccc(-c2nc(-c3ccc(C)cc3)nc(-c3cc4nc(-c5ccccc5)[se]c4c4ccccc34)n2)cc1]

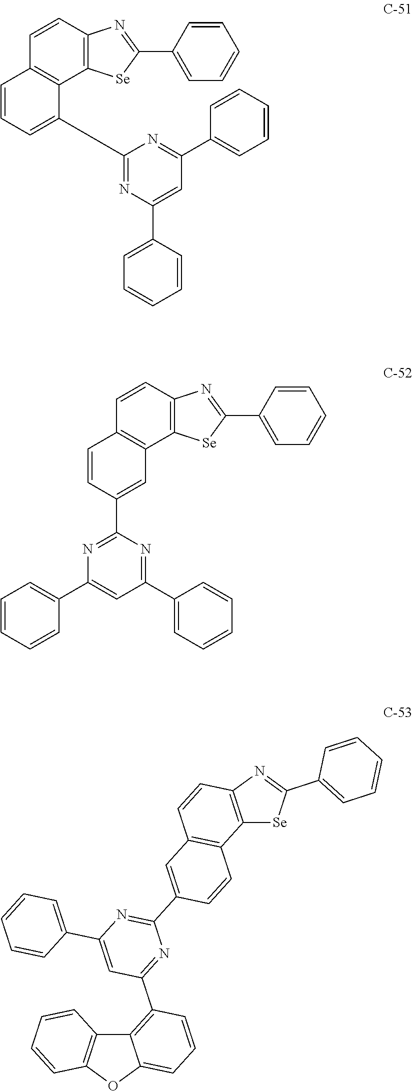 [SMILES: c1ccc(-c2cc(-c3ccccc3)nc(-c3cccc4ccc5nc(-c6ccccc6)[se]c5c34)n2)cc1 ; c1ccc(-c2cc(-c3ccccc3)nc(-c3ccc4ccc5nc(-c6ccccc6)[se]c5c4c3)n2)cc1 ; c1ccc(-c2cc(-c3cccc4oc5ccccc5c34)nc(-c3ccc4c(ccc5nc(-c6ccccc6)[se]c54)c3)n2)cc1]

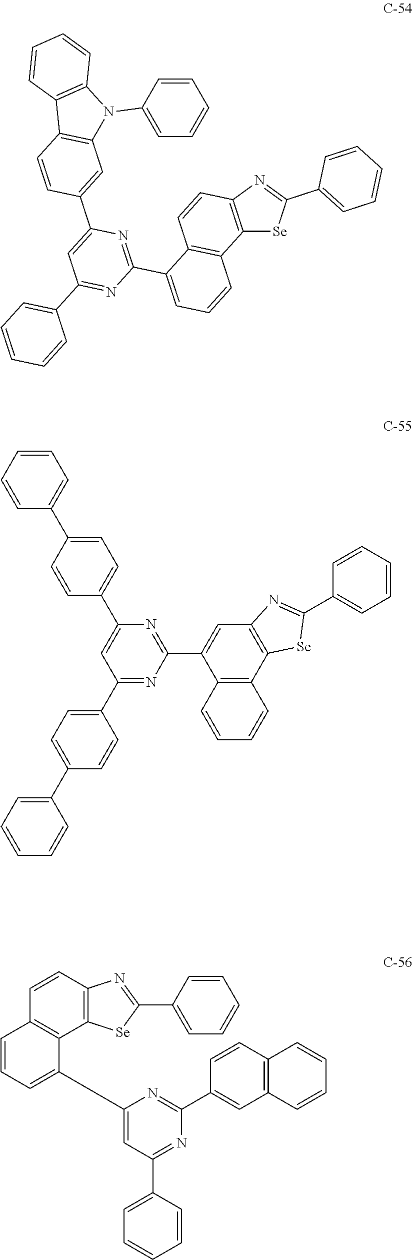 [SMILES: c1ccc(-c2cc(-c3ccc4c5ccccc5n(-c5ccccc5)c4c3)nc(-c3cccc4c3ccc3nc(-c5ccccc5)[se]c34)n2)cc1 ; c1ccc(-c2ccc(-c3cc(-c4ccc(-c5ccccc5)cc4)nc(-c4cc5nc(-c6ccccc6)[se]c5c5ccccc45)n3)cc2)cc1 ; c1ccc(-c2cc(-c3cccc4ccc5nc(-c6ccccc6)[se]c5c34)nc(-c3ccc4ccccc4c3)n2)cc1]

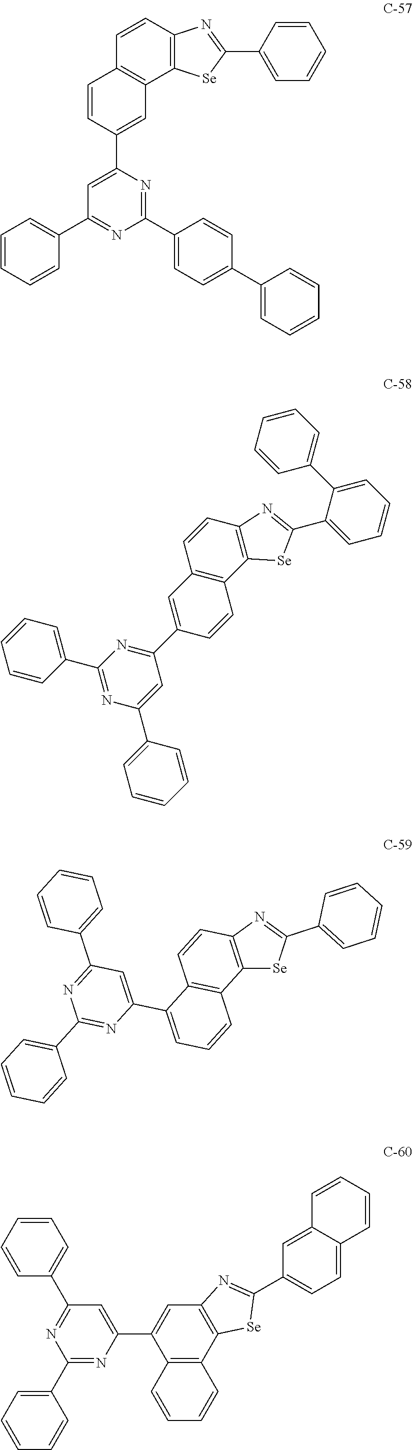 [SMILES: c1ccc(-c2ccc(-c3nc(-c4ccccc4)cc(-c4ccc5ccc6nc(-c7ccccc7)[se]c6c5c4)n3)cc2)cc1 ; c1ccc(-c2cc(-c3ccc4c(ccc5nc(-c6ccccc6-c6ccccc6)[se]c54)c3)nc(-c3ccccc3)n2)cc1 ; c1ccc(-c2cc(-c3cccc4c3ccc3nc(-c5ccccc5)[se]c34)nc(-c3ccccc3)n2)cc1 ; c1ccc(-c2cc(-c3cc4nc(-c5ccc6ccccc6c5)[se]c4c4ccccc34)nc(-c3ccccc3)n2)cc1]

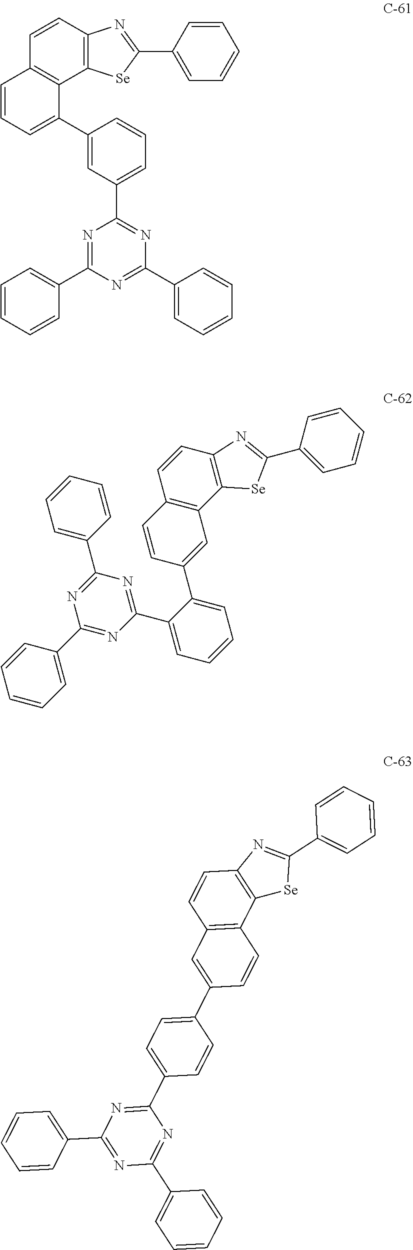 [SMILES: c1ccc(-c2nc(-c3ccccc3)nc(-c3cccc(-c4cccc5ccc6nc(-c7ccccc7)[se]c6c45)c3)n2)cc1 ; c1ccc(-c2nc(-c3ccccc3)nc(-c3ccccc3-c3ccc4ccc5nc(-c6ccccc6)[se]c5c4c3)n2)cc1 ; c1ccc(-c2nc(-c3ccccc3)nc(-c3ccc(-c4ccc5c(ccc6nc(-c7ccccc7)[se]c65)c4)cc3)n2)cc1]

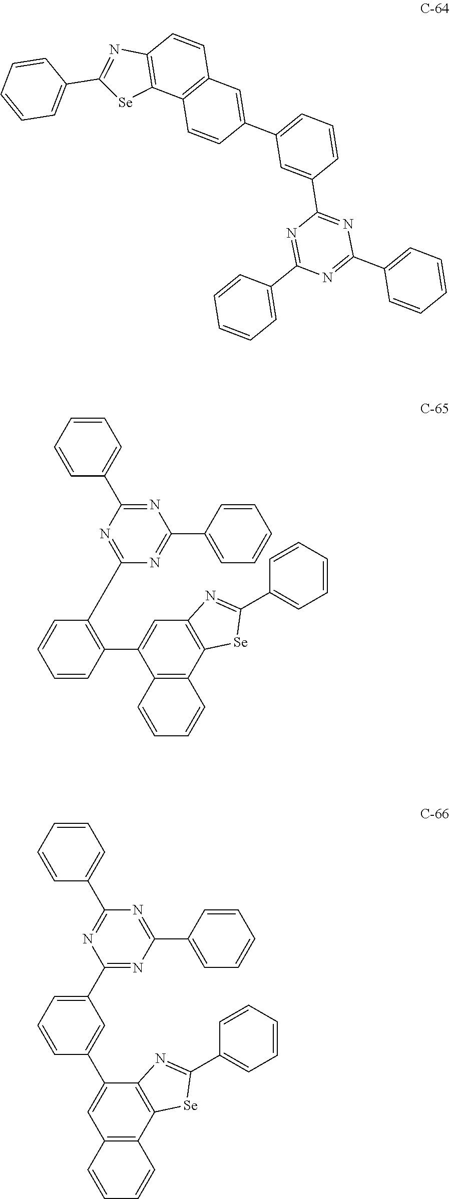 [SMILES: c1ccc(-c2nc(-c3ccccc3)nc(-c3cccc(-c4ccc5c(ccc6nc(-c7ccccc7)[se]c65)c4)c3)n2)cc1 ; c1ccc(-c2nc(-c3ccccc3)nc(-c3ccccc3-c3cc4nc(-c5ccccc5)[se]c4c4ccccc34)n2)cc1 ; c1ccc(-c2nc(-c3ccccc3)nc(-c3cccc(-c4cc5ccccc5c5[se]c(-c6ccccc6)nc45)c3)n2)cc1]

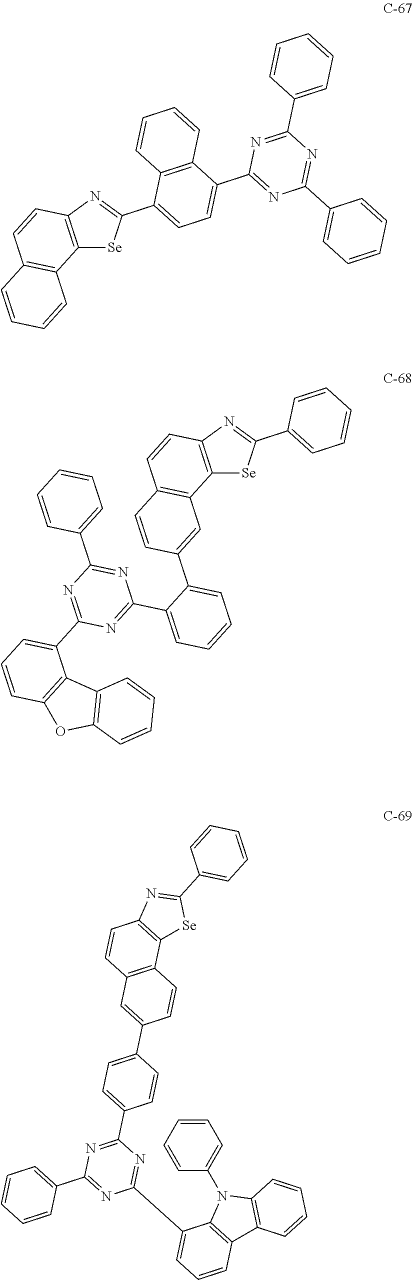 [SMILES: c1ccc(-c2nc(-c3ccccc3)nc(-c3ccc(-c4nc5ccc6ccccc6c5[se]4)c4ccccc34)n2)cc1 ; c1ccc(-c2nc(-c3ccccc3-c3ccc4ccc5nc(-c6ccccc6)[se]c5c4c3)nc(-c3cccc4oc5ccccc5c34)n2)cc1 ; c1ccc(-c2nc(-c3ccc(-c4ccc5c(ccc6nc(-c7ccccc7)[se]c65)c4)cc3)nc(-c3cccc4c5ccccc5n(-c5ccccc5)c34)n2)cc1]

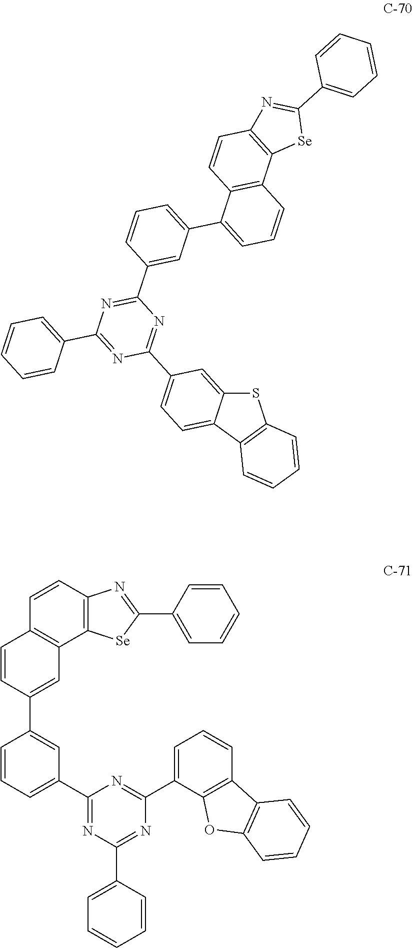 [SMILES: c1ccc(-c2nc(-c3cccc(-c4cccc5c4ccc4nc(-c6ccccc6)[se]c45)c3)nc(-c3ccc4c(c3)sc3ccccc34)n2)cc1 ; c1ccc(-c2nc(-c3cccc(-c4ccc5ccc6nc(-c7ccccc7)[se]c6c5c4)c3)nc(-c3cccc4c3oc3ccccc34)n2)cc1]

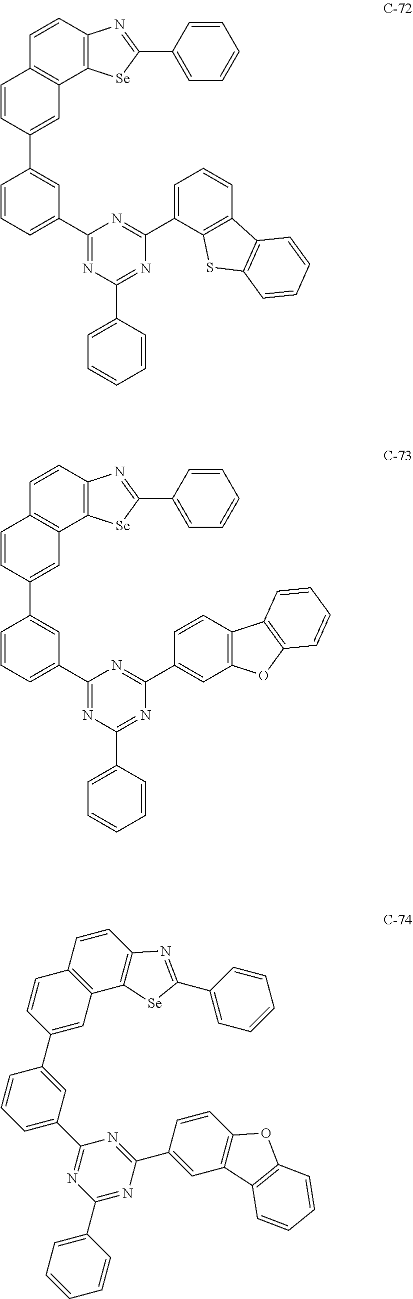 [SMILES: c1ccc(-c2nc(-c3cccc(-c4ccc5ccc6nc(-c7ccccc7)[se]c6c5c4)c3)nc(-c3cccc4c3sc3ccccc34)n2)cc1 ; c1ccc(-c2nc(-c3cccc(-c4ccc5ccc6nc(-c7ccccc7)[se]c6c5c4)c3)nc(-c3ccc4c(c3)oc3ccccc34)n2)cc1 ; c1ccc(-c2nc(-c3cccc(-c4ccc5ccc6nc(-c7ccccc7)[se]c6c5c4)c3)nc(-c3ccc4oc5ccccc5c4c3)n2)cc1]

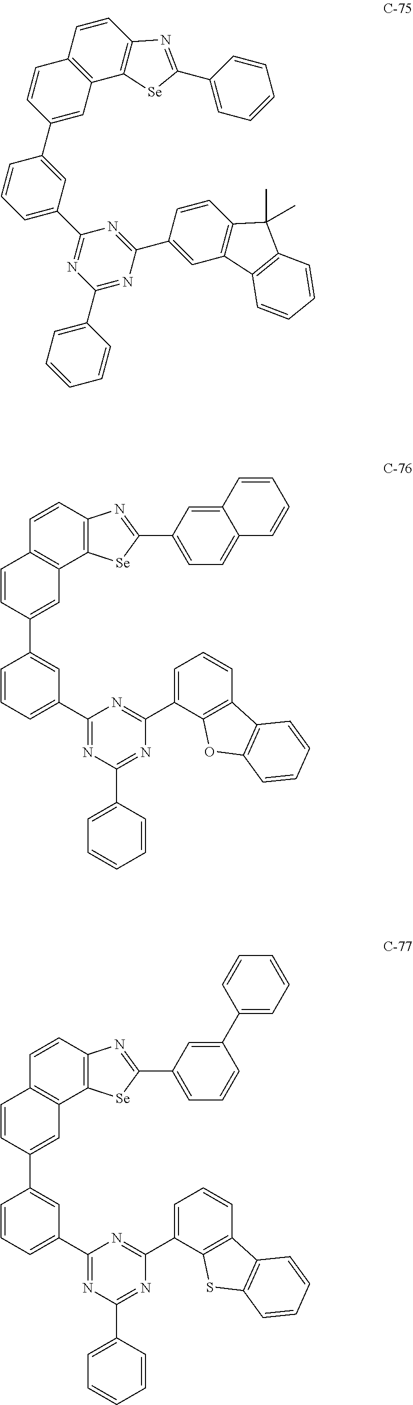 [SMILES: CC1(C)c2ccccc2-c2cc(-c3nc(-c4ccccc4)nc(-c4cccc(-c5ccc6ccc7nc(-c8ccccc8)[se]c7c6c5)c4)n3)ccc21 ; c1ccc(-c2nc(-c3cccc(-c4ccc5ccc6nc(-c7ccc8ccccc8c7)[se]c6c5c4)c3)nc(-c3cccc4c3oc3ccccc34)n2)cc1 ; c1ccc(-c2cccc(-c3nc4ccc5ccc(-c6cccc(-c7nc(-c8ccccc8)nc(-c8cccc9c8sc8ccccc89)n7)c6)cc5c4[se]3)c2)cc1]

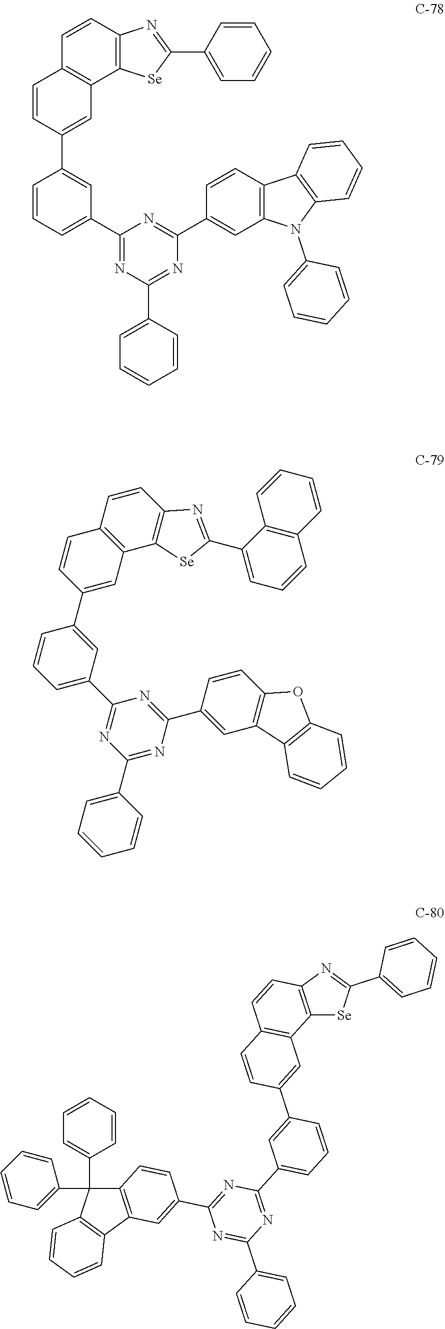 [SMILES: c1ccc(-c2nc(-c3cccc(-c4ccc5ccc6nc(-c7ccccc7)[se]c6c5c4)c3)nc(-c3ccc4c5ccccc5n(-c5ccccc5)c4c3)n2)cc1 ; c1ccc(-c2nc(-c3cccc(-c4ccc5ccc6nc(-c7cccc8ccccc78)[se]c6c5c4)c3)nc(-c3ccc4oc5ccccc5c4c3)n2)cc1 ; c1ccc(-c2nc(-c3cccc(-c4ccc5ccc6nc(-c7ccccc7)[se]c6c5c4)c3)nc(-c3ccc4c(c3)-c3ccccc3C4(c3ccccc3)c3ccccc3)n2)cc1]

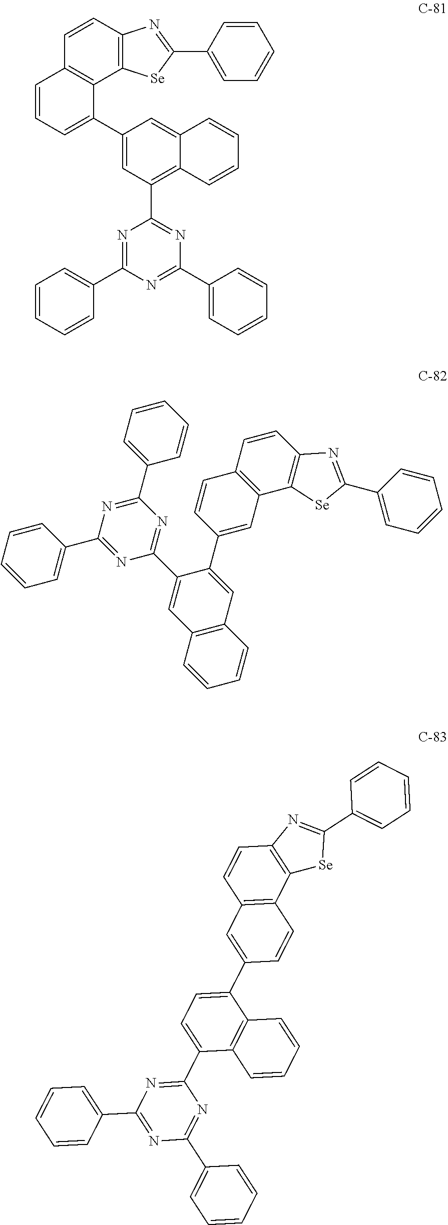 [SMILES: c1ccc(-c2nc(-c3ccccc3)nc(-c3cc(-c4cccc5ccc6nc(-c7ccccc7)[se]c6c45)cc4ccccc34)n2)cc1 ; c1ccc(-c2nc(-c3ccccc3)nc(-c3cc4ccccc4cc3-c3ccc4ccc5nc(-c6ccccc6)[se]c5c4c3)n2)cc1 ; c1ccc(-c2nc(-c3ccccc3)nc(-c3ccc(-c4ccc5c(ccc6nc(-c7ccccc7)[se]c65)c4)c4ccccc34)n2)cc1]

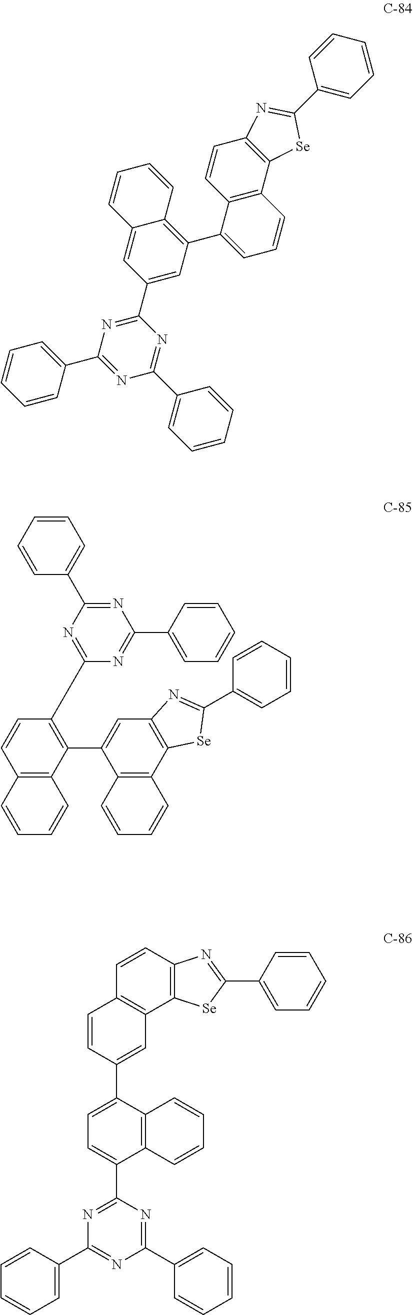 [SMILES: c1ccc(-c2nc(-c3ccccc3)nc(-c3cc(-c4cccc5c4ccc4nc(-c6ccccc6)[se]c45)c4ccccc4c3)n2)cc1 ; c1ccc(-c2nc(-c3ccccc3)nc(-c3ccc4ccccc4c3-c3cc4nc(-c5ccccc5)[se]c4c4ccccc34)n2)cc1 ; c1ccc(-c2nc(-c3ccccc3)nc(-c3ccc(-c4ccc5ccc6nc(-c7ccccc7)[se]c6c5c4)c4ccccc34)n2)cc1]

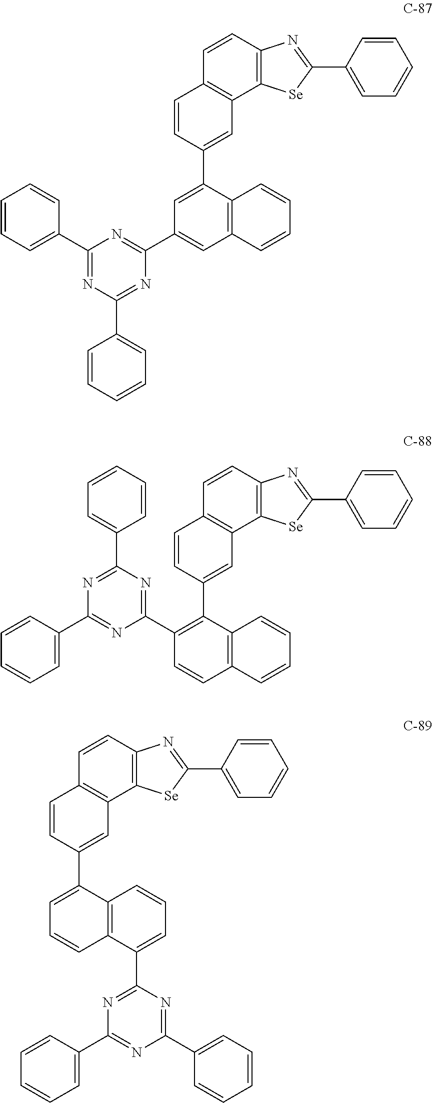 [SMILES: c1ccc(-c2nc(-c3ccccc3)nc(-c3cc(-c4ccc5ccc6nc(-c7ccccc7)[se]c6c5c4)c4ccccc4c3)n2)cc1 ; c1ccc(-c2nc(-c3ccccc3)nc(-c3ccc4ccccc4c3-c3ccc4ccc5nc(-c6ccccc6)[se]c5c4c3)n2)cc1 ; c1ccc(-c2nc(-c3ccccc3)nc(-c3cccc4c(-c5ccc6ccc7nc(-c8ccccc8)[se]c7c6c5)cccc34)n2)cc1]

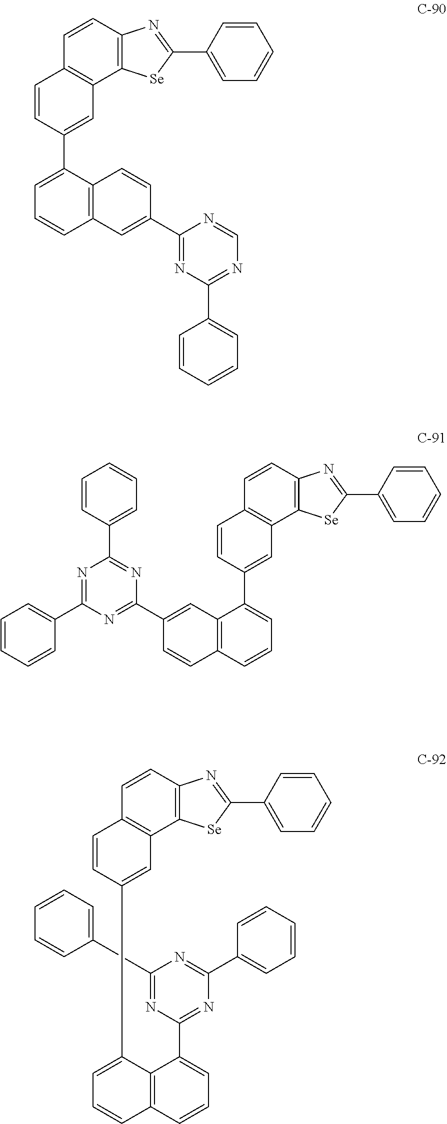 [SMILES: c1ccc(-c2ncnc(-c3ccc4c(-c5ccc6ccc7nc(-c8ccccc8)[se]c7c6c5)cccc4c3)n2)cc1 ; c1ccc(-c2nc(-c3ccccc3)nc(-c3ccc4cccc(-c5ccc6ccc7nc(-c8ccccc8)[se]c7c6c5)c4c3)n2)cc1 ; c1ccc(-c2nc(-c3ccccc3)nc(-c3cccc4cccc(-c5ccc6ccc7nc(-c8ccccc8)[se]c7c6c5)c34)n2)cc1]

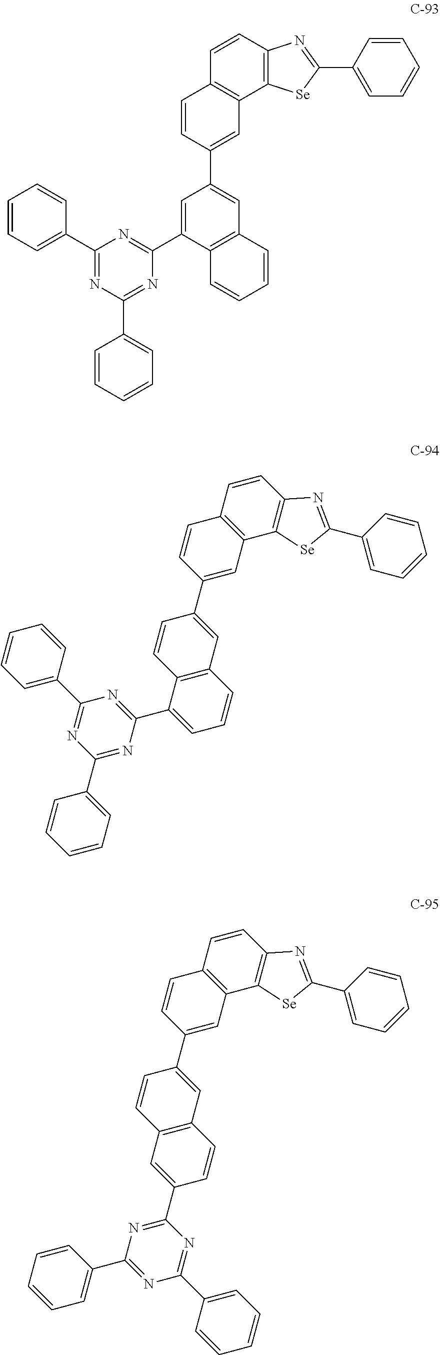 [SMILES: c1ccc(-c2nc(-c3ccccc3)nc(-c3cc(-c4ccc5ccc6nc(-c7ccccc7)[se]c6c5c4)cc4ccccc34)n2)cc1 ; c1ccc(-c2nc(-c3ccccc3)nc(-c3cccc4cc(-c5ccc6ccc7nc(-c8ccccc8)[se]c7c6c5)ccc34)n2)cc1 ; c1ccc(-c2nc(-c3ccccc3)nc(-c3ccc4cc(-c5ccc6ccc7nc(-c8ccccc8)[se]c7c6c5)ccc4c3)n2)cc1]

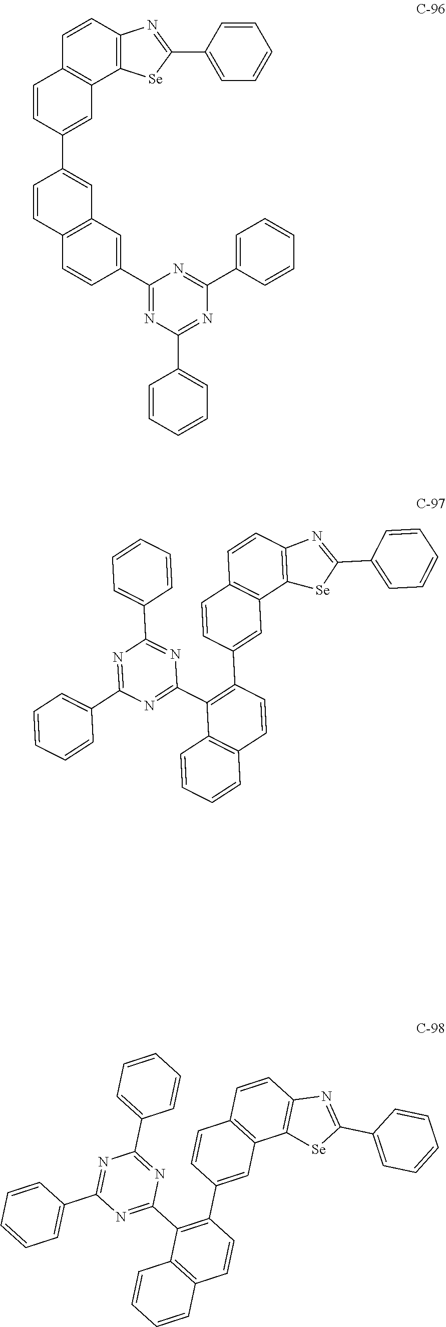 [SMILES: c1ccc(-c2nc(-c3ccccc3)nc(-c3ccc4ccc(-c5ccc6ccc7nc(-c8ccccc8)[se]c7c6c5)cc4c3)n2)cc1 ; c1ccc(-c2nc(-c3ccccc3)nc(-c3c(-c4ccc5ccc6nc(-c7ccccc7)[se]c6c5c4)ccc4ccccc34)n2)cc1 ; c1ccc(-c2nc(-c3ccccc3)nc(-c3c(-c4ccc5ccc6nc(-c7ccccc7)[se]c6c5c4)ccc4ccccc34)n2)cc1]

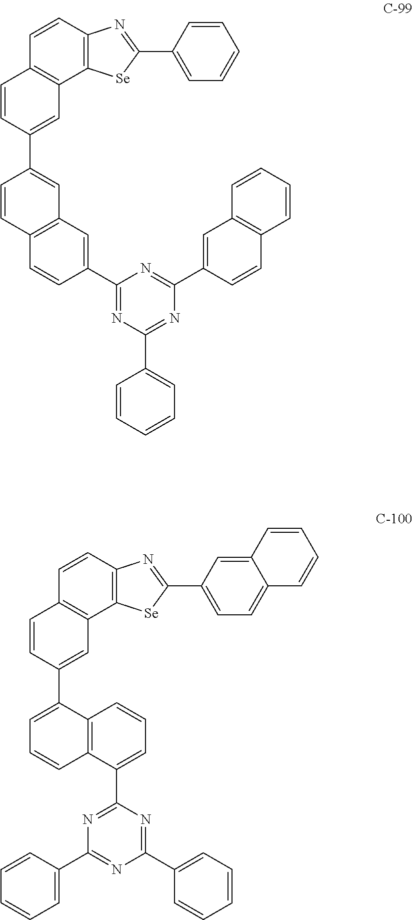 [SMILES: c1ccc(-c2nc(-c3ccc4ccccc4c3)nc(-c3ccc4ccc(-c5ccc6ccc7nc(-c8ccccc8)[se]c7c6c5)cc4c3)n2)cc1 ; c1ccc(-c2nc(-c3ccccc3)nc(-c3cccc4c(-c5ccc6ccc7nc(-c8ccc9ccccc9c8)[se]c7c6c5)cccc34)n2)cc1]

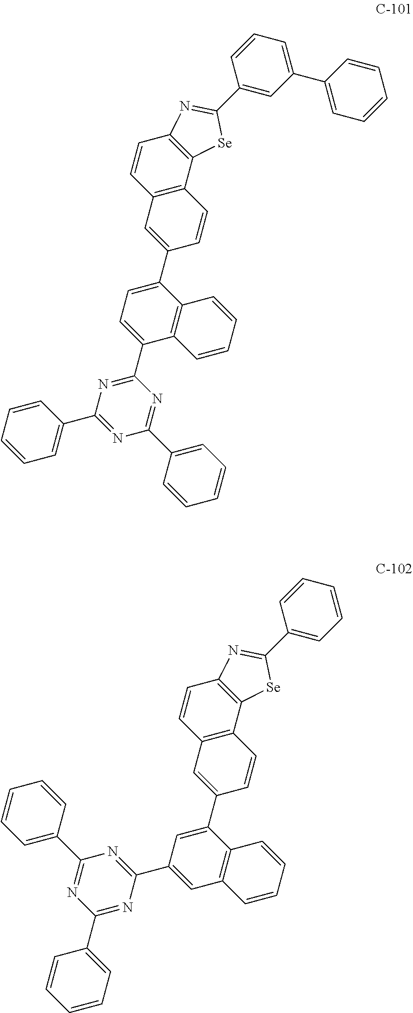 [SMILES: c1ccc(-c2cccc(-c3nc4ccc5cc(-c6ccc(-c7nc(-c8ccccc8)nc(-c8ccccc8)n7)c7ccccc67)ccc5c4[se]3)c2)cc1 ; c1ccc(-c2nc(-c3ccccc3)nc(-c3cc(-c4ccc5c(ccc6nc(-c7ccccc7)[se]c65)c4)c4ccccc4c3)n2)cc1]

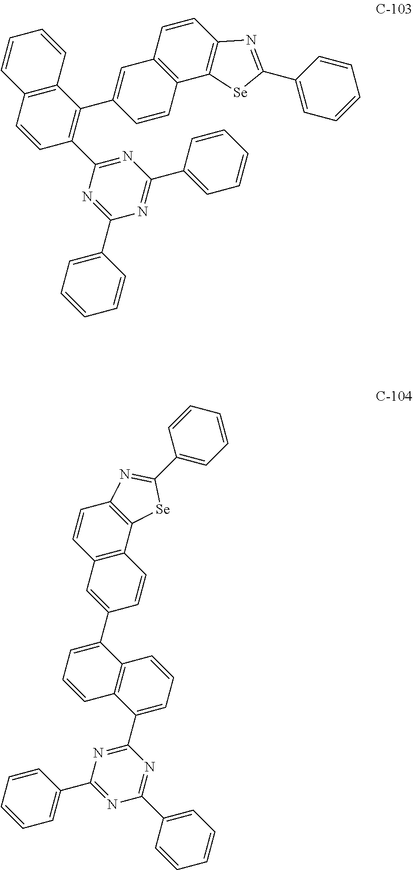 [SMILES: c1ccc(-c2nc(-c3ccccc3)nc(-c3ccc4ccccc4c3-c3ccc4c(ccc5nc(-c6ccccc6)[se]c54)c3)n2)cc1 ; c1ccc(-c2nc(-c3ccccc3)nc(-c3cccc4c(-c5ccc6c(ccc7nc(-c8ccccc8)[se]c76)c5)cccc34)n2)cc1]

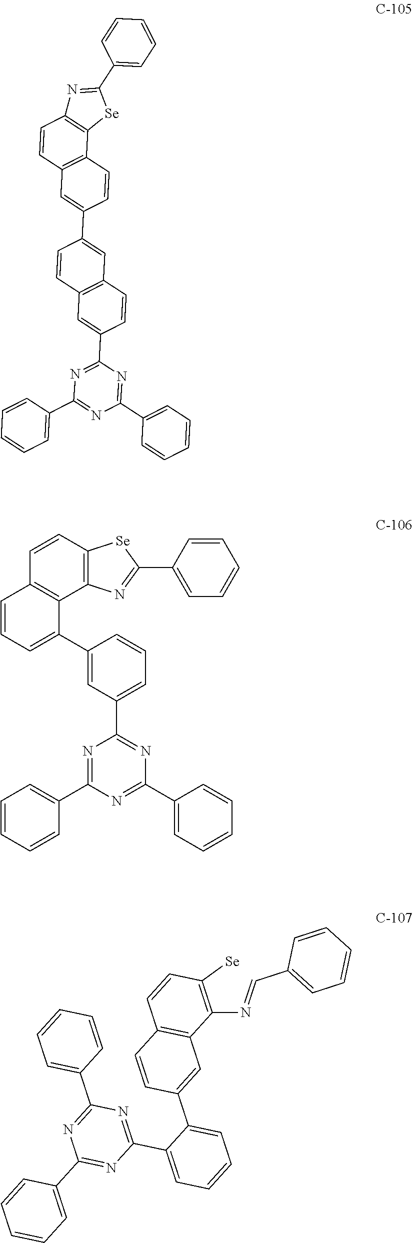

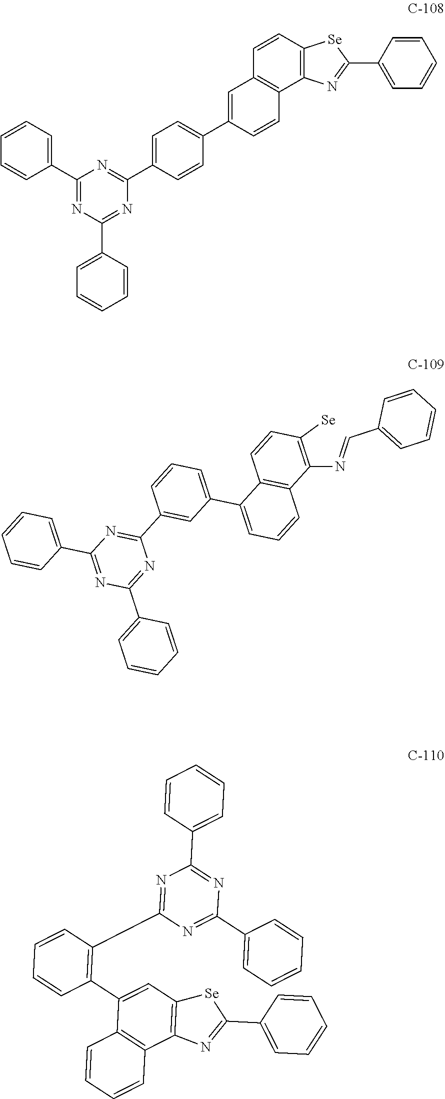

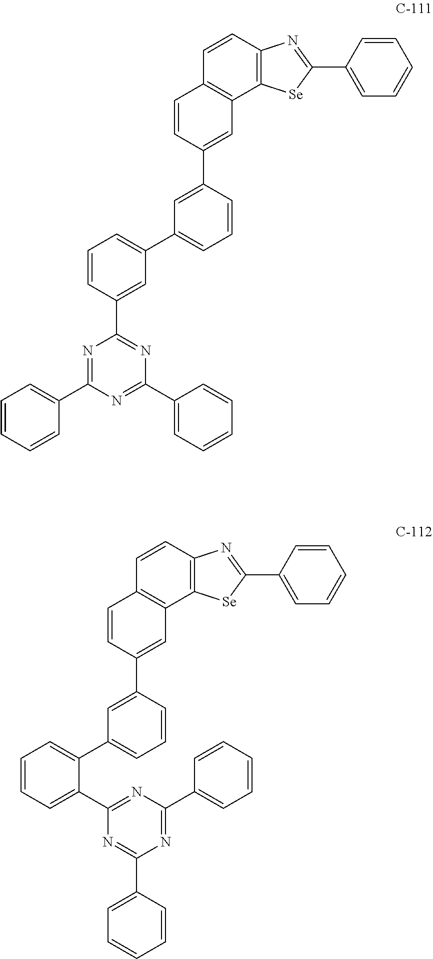 [SMILES: c1ccc(-c2nc(-c3ccccc3)nc(-c3cccc(-c4cccc(-c5ccc6ccc7nc(-c8ccccc8)[se]c7c6c5)c4)c3)n2)cc1 ; c1ccc(-c2nc(-c3ccccc3)nc(-c3ccccc3-c3cccc(-c4ccc5ccc6nc(-c7ccccc7)[se]c6c5c4)c3)n2)cc1]

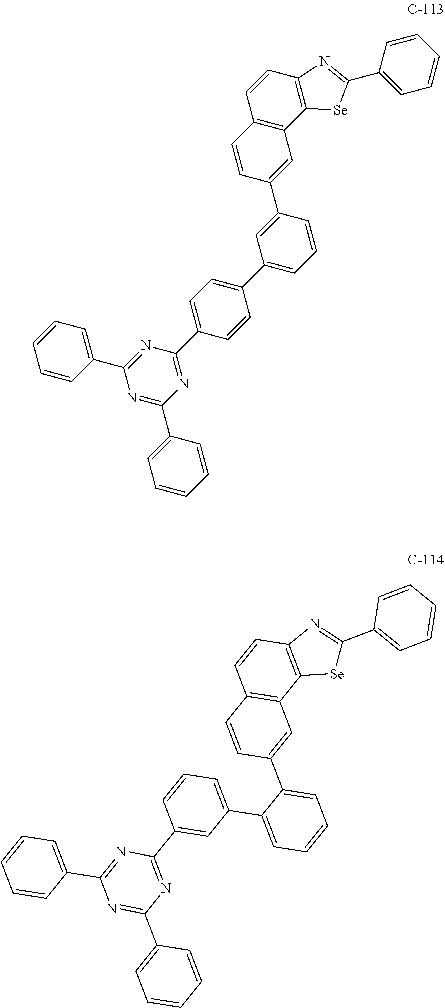 [SMILES: c1ccc(-c2nc(-c3ccccc3)nc(-c3ccc(-c4cccc(-c5ccc6ccc7nc(-c8ccccc8)[se]c7c6c5)c4)cc3)n2)cc1 ; c1ccc(-c2nc(-c3ccccc3)nc(-c3cccc(-c4ccccc4-c4ccc5ccc6nc(-c7ccccc7)[se]c6c5c4)c3)n2)cc1]

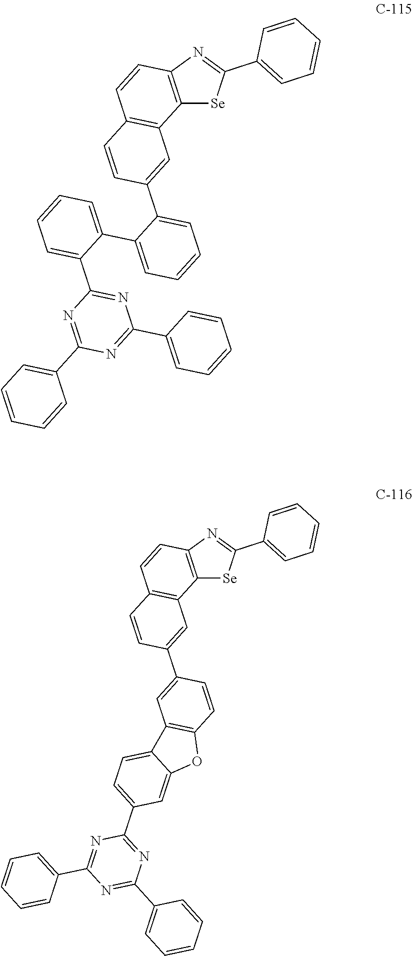 [SMILES: c1ccc(-c2nc(-c3ccccc3)nc(-c3ccccc3-c3ccccc3-c3ccc4ccc5nc(-c6ccccc6)[se]c5c4c3)n2)cc1 ; c1ccc(-c2nc(-c3ccccc3)nc(-c3ccc4c(c3)oc3ccc(-c5ccc6ccc7nc(-c8ccccc8)[se]c7c6c5)cc34)n2)cc1]

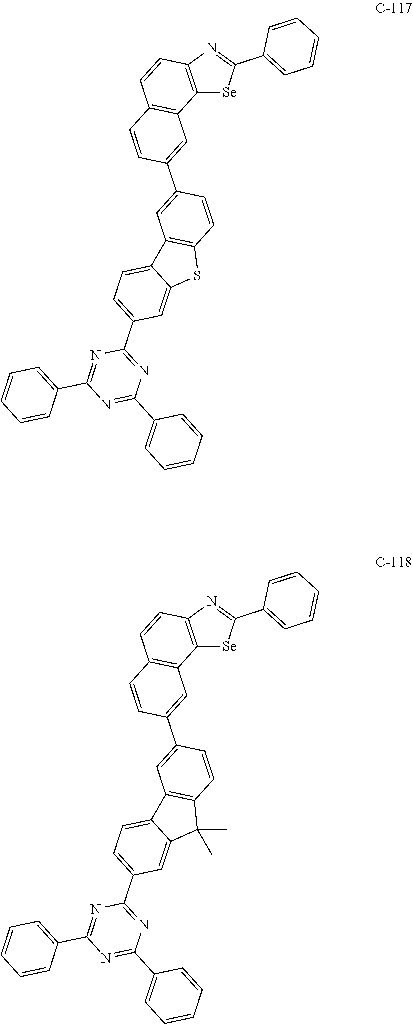 [SMILES: c1ccc(-c2nc(-c3ccccc3)nc(-c3ccc4c(c3)sc3ccc(-c5ccc6ccc7nc(-c8ccccc8)[se]c7c6c5)cc34)n2)cc1 ; CC1(C)c2ccc(-c3ccc4ccc5nc(-c6ccccc6)[se]c5c4c3)cc2-c2ccc(-c3nc(-c4ccccc4)nc(-c4ccccc4)n3)cc21]

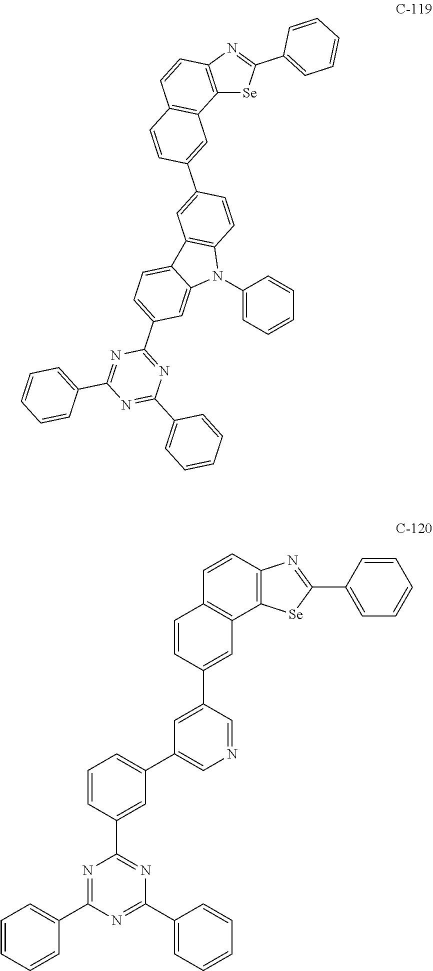 [SMILES: c1ccc(-c2nc(-c3ccccc3)nc(-c3ccc4c5cc(-c6ccc7ccc8nc(-c9ccccc9)[se]c8c7c6)ccc5n(-c5ccccc5)c4c3)n2)cc1 ; c1ccc(-c2nc(-c3ccccc3)nc(-c3cccc(-c4cncc(-c5ccc6ccc7nc(-c8ccccc8)[se]c7c6c5)c4)c3)n2)cc1]

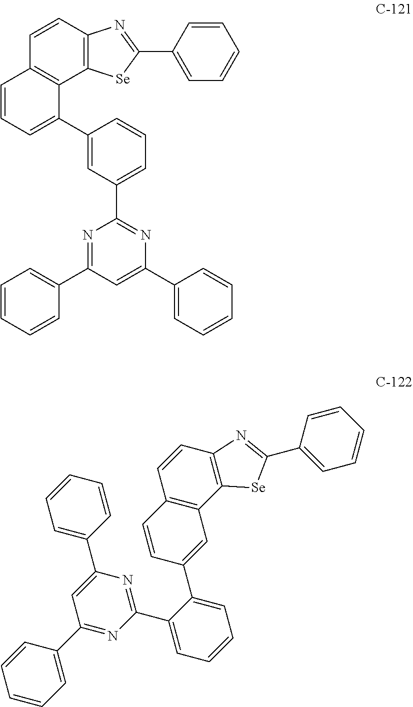 [SMILES: c1ccc(-c2cc(-c3ccccc3)nc(-c3cccc(-c4cccc5ccc6nc(-c7ccccc7)[se]c6c45)c3)n2)cc1 ; c1ccc(-c2cc(-c3ccccc3)nc(-c3ccccc3-c3ccc4ccc5nc(-c6ccccc6)[se]c5c4c3)n2)cc1]

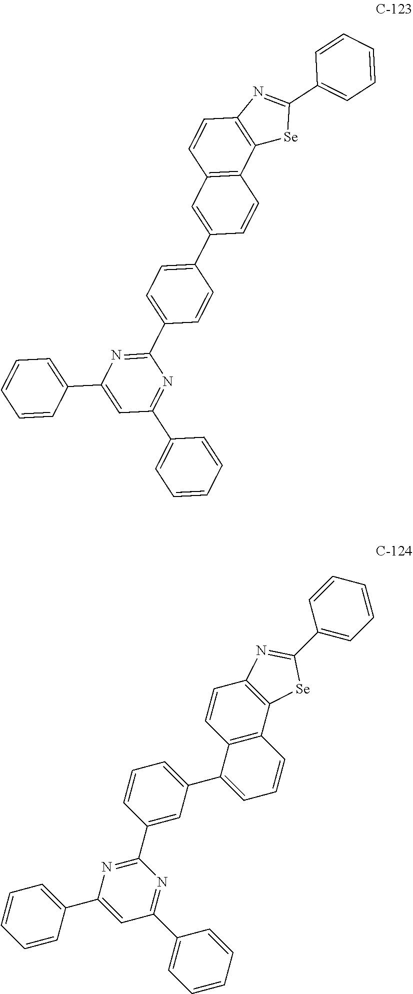 [SMILES: c1ccc(-c2cc(-c3ccccc3)nc(-c3ccc(-c4ccc5c(ccc6nc(-c7ccccc7)[se]c65)c4)cc3)n2)cc1 ; c1ccc(-c2cc(-c3ccccc3)nc(-c3cccc(-c4cccc5c4ccc4nc(-c6ccccc6)[se]c45)c3)n2)cc1]

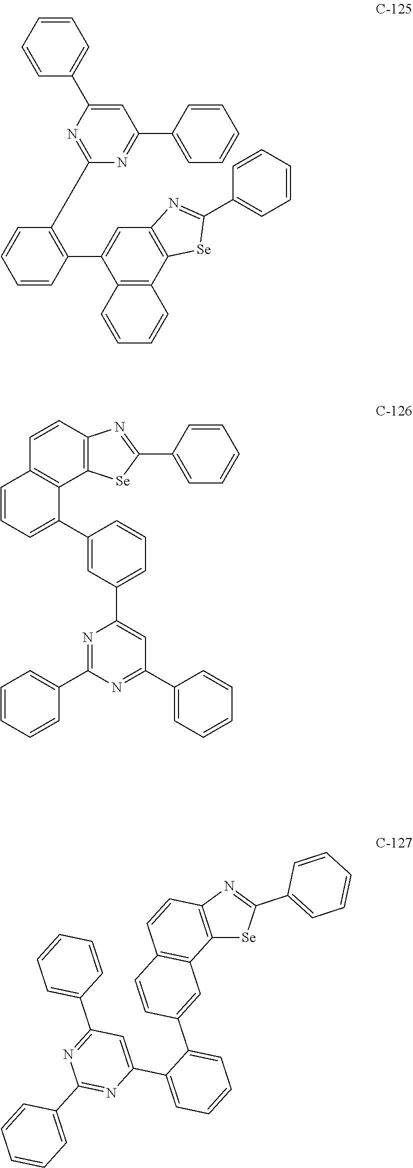 [SMILES: c1ccc(-c2cc(-c3ccccc3)nc(-c3ccccc3-c3cc4nc(-c5ccccc5)[se]c4c4ccccc34)n2)cc1 ; c1ccc(-c2cc(-c3cccc(-c4cccc5ccc6nc(-c7ccccc7)[se]c6c45)c3)nc(-c3ccccc3)n2)cc1 ; c1ccc(-c2cc(-c3ccccc3-c3ccc4ccc5nc(-c6ccccc6)[se]c5c4c3)nc(-c3ccccc3)n2)cc1]

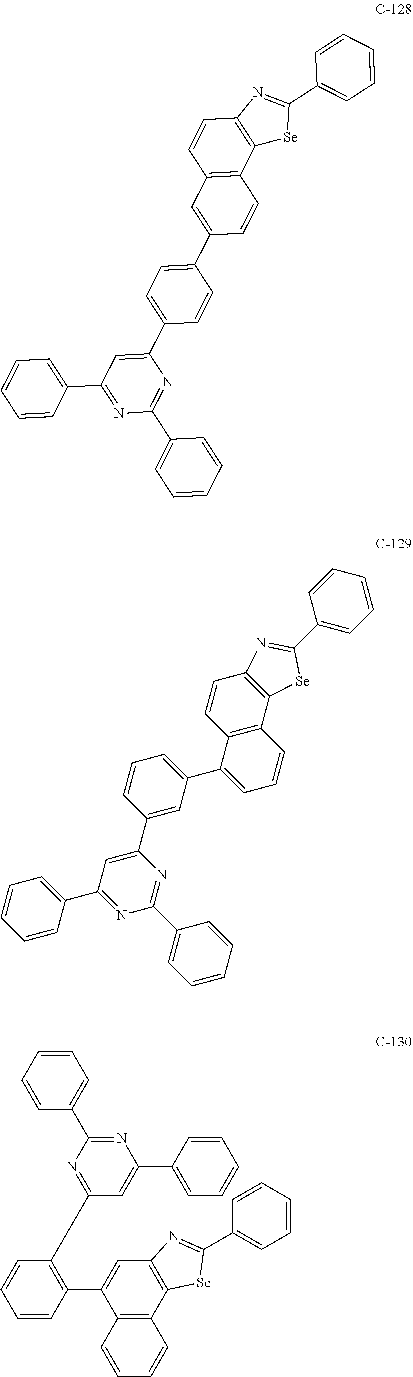 [SMILES: c1ccc(-c2cc(-c3ccc(-c4ccc5c(ccc6nc(-c7ccccc7)[se]c65)c4)cc3)nc(-c3ccccc3)n2)cc1 ; c1ccc(-c2cc(-c3cccc(-c4cccc5c4ccc4nc(-c6ccccc6)[se]c45)c3)nc(-c3ccccc3)n2)cc1 ; c1ccc(-c2cc(-c3ccccc3-c3cc4nc(-c5ccccc5)[se]c4c4ccccc34)nc(-c3ccccc3)n2)cc1]

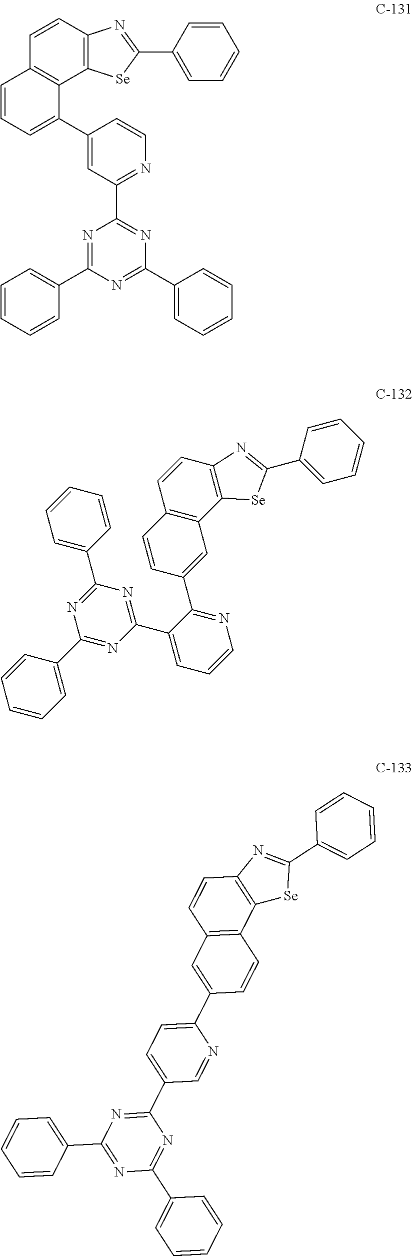 [SMILES: c1ccc(-c2nc(-c3ccccc3)nc(-c3cc(-c4cccc5ccc6nc(-c7ccccc7)[se]c6c45)ccn3)n2)cc1 ; c1ccc(-c2nc(-c3ccccc3)nc(-c3cccnc3-c3ccc4ccc5nc(-c6ccccc6)[se]c5c4c3)n2)cc1 ; c1ccc(-c2nc(-c3ccccc3)nc(-c3ccc(-c4ccc5c(ccc6nc(-c7ccccc7)[se]c65)c4)nc3)n2)cc1]

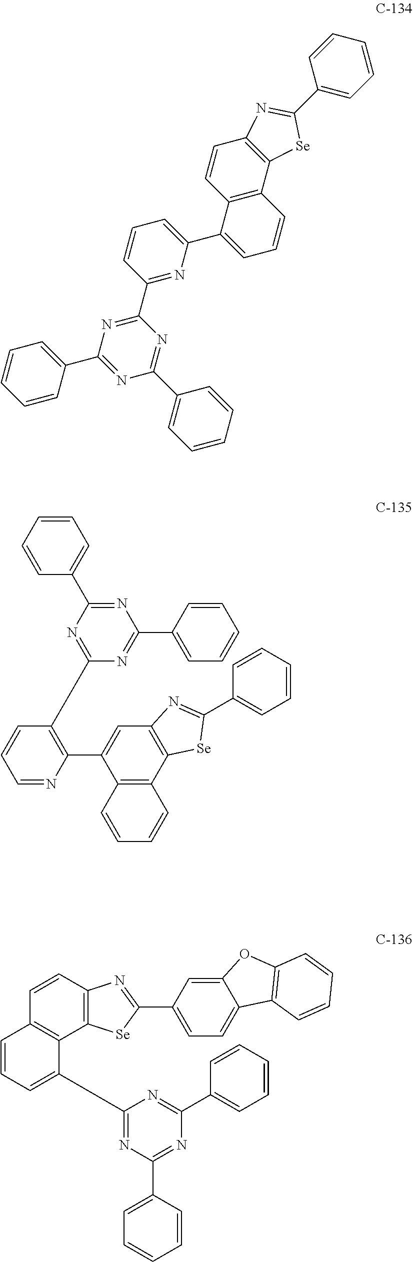 [SMILES: c1ccc(-c2nc(-c3ccccc3)nc(-c3cccc(-c4cccc5c4ccc4nc(-c6ccccc6)[se]c45)n3)n2)cc1 ; c1ccc(-c2nc(-c3ccccc3)nc(-c3cccnc3-c3cc4nc(-c5ccccc5)[se]c4c4ccccc34)n2)cc1 ; c1ccc(-c2nc(-c3ccccc3)nc(-c3cccc4ccc5nc(-c6ccc7c(c6)oc6ccccc67)[se]c5c34)n2)cc1]

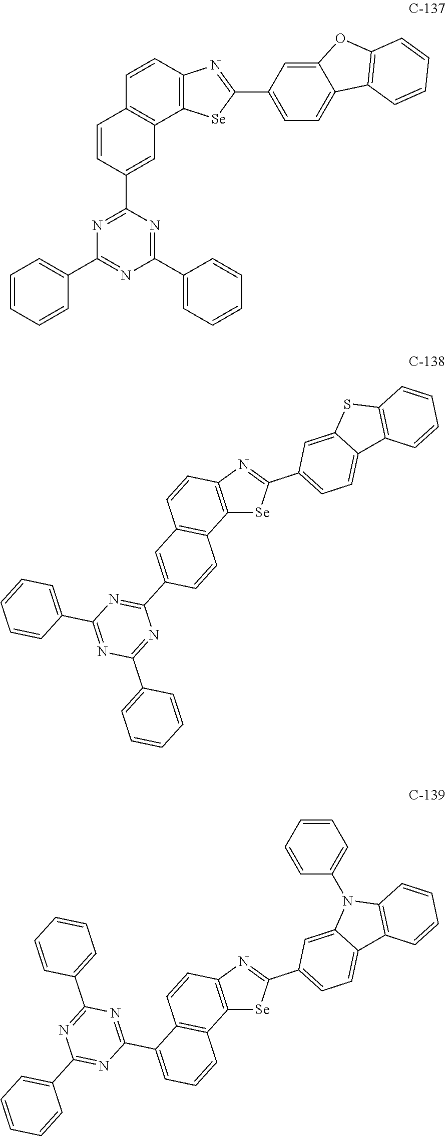 [SMILES: c1ccc(-c2nc(-c3ccccc3)nc(-c3ccc4ccc5nc(-c6ccc7c(c6)oc6ccccc67)[se]c5c4c3)n2)cc1 ; c1ccc(-c2nc(-c3ccccc3)nc(-c3ccc4c(ccc5nc(-c6ccc7c(c6)sc6ccccc67)[se]c54)c3)n2)cc1 ; c1ccc(-c2nc(-c3ccccc3)nc(-c3cccc4c3ccc3nc(-c5ccc6c7ccccc7n(-c7ccccc7)c6c5)[se]c34)n2)cc1]

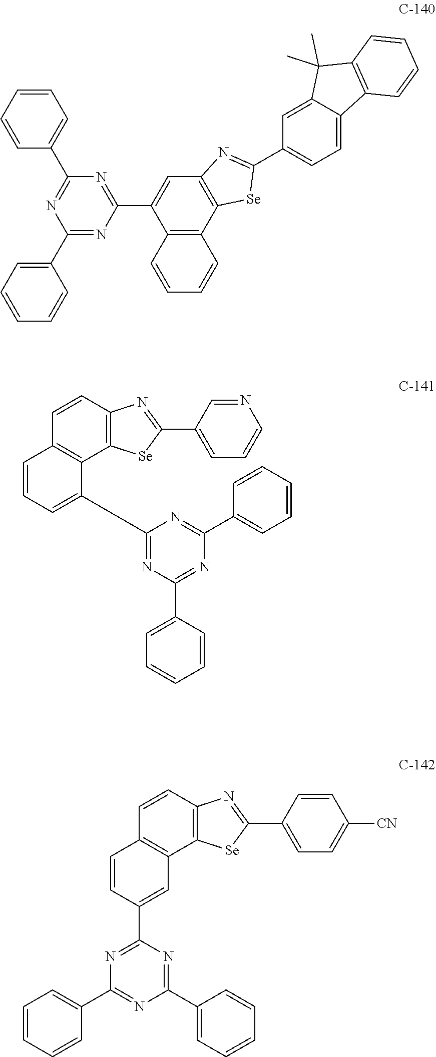 [SMILES: CC1(C)c2ccccc2-c2ccc(-c3nc4cc(-c5nc(-c6ccccc6)nc(-c6ccccc6)n5)c5ccccc5c4[se]3)cc21 ; c1ccc(-c2nc(-c3ccccc3)nc(-c3cccc4ccc5nc(-c6cccnc6)[se]c5c34)n2)cc1 ; N#Cc1ccc(-c2nc3ccc4ccc(-c5nc(-c6ccccc6)nc(-c6ccccc6)n5)cc4c3[se]2)cc1]

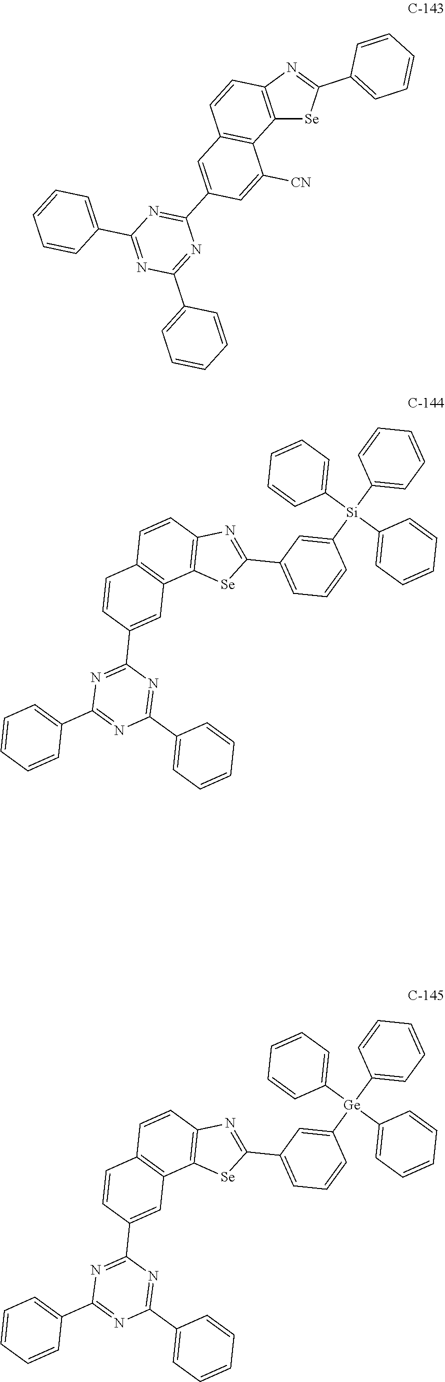

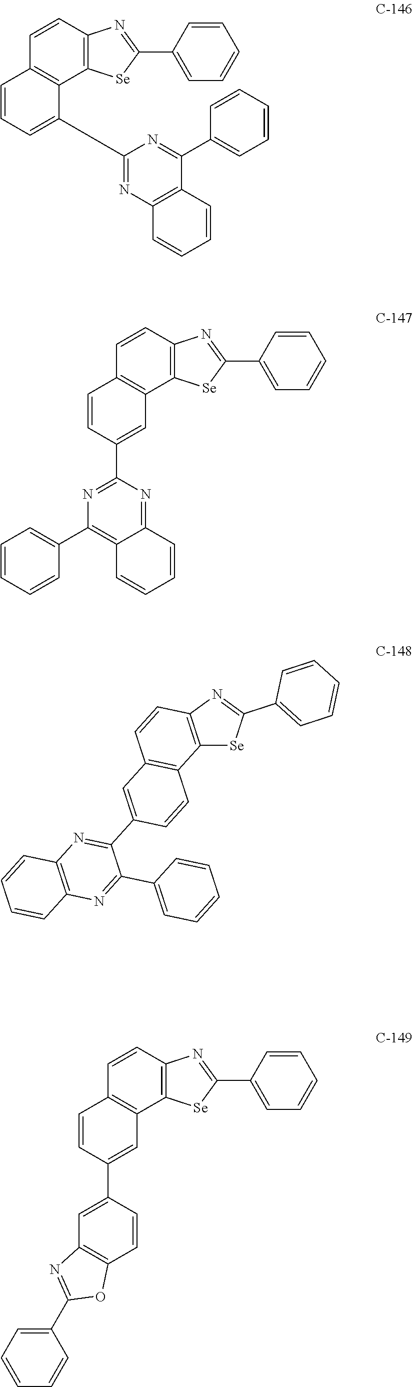 [SMILES: c1ccc(-c2nc3ccc4cccc(-c5nc(-c6ccccc6)c6ccccc6n5)c4c3[se]2)cc1 ; c1ccc(-c2nc3ccc4ccc(-c5nc(-c6ccccc6)c6ccccc6n5)cc4c3[se]2)cc1 ; c1ccc(-c2nc3ccc4cc(-c5nc6ccccc6nc5-c5ccccc5)ccc4c3[se]2)cc1 ; c1ccc(-c2nc3cc(-c4ccc5ccc6nc(-c7ccccc7)[se]c6c5c4)ccc3o2)cc1]

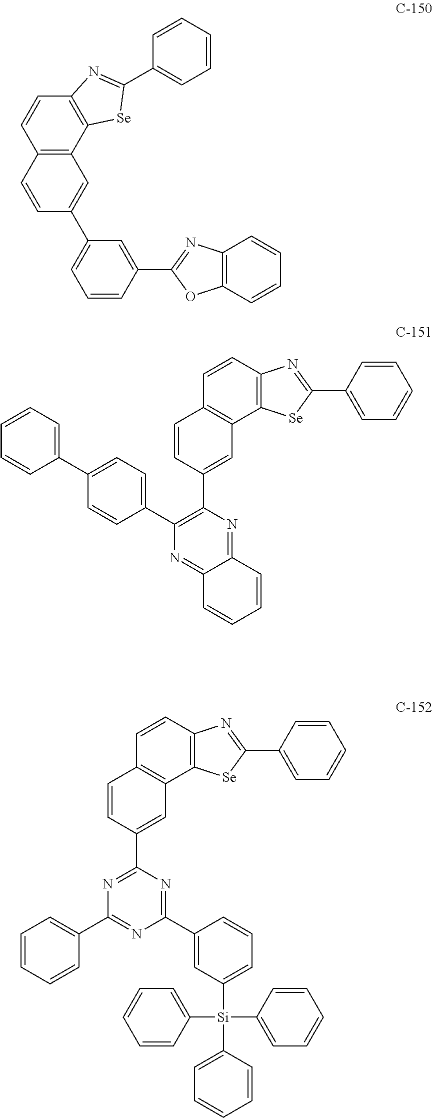 [SMILES: c1ccc(-c2nc3ccc4ccc(-c5cccc(-c6nc7ccccc7o6)c5)cc4c3[se]2)cc1 ; c1ccc(-c2ccc(-c3nc4ccccc4nc3-c3ccc4ccc5nc(-c6ccccc6)[se]c5c4c3)cc2)cc1 ; c1ccc(-c2nc(-c3cccc([Si](c4ccccc4)(c4ccccc4)c4ccccc4)c3)nc(-c3ccc4ccc5nc(-c6ccccc6)[se]c5c4c3)n2)cc1]

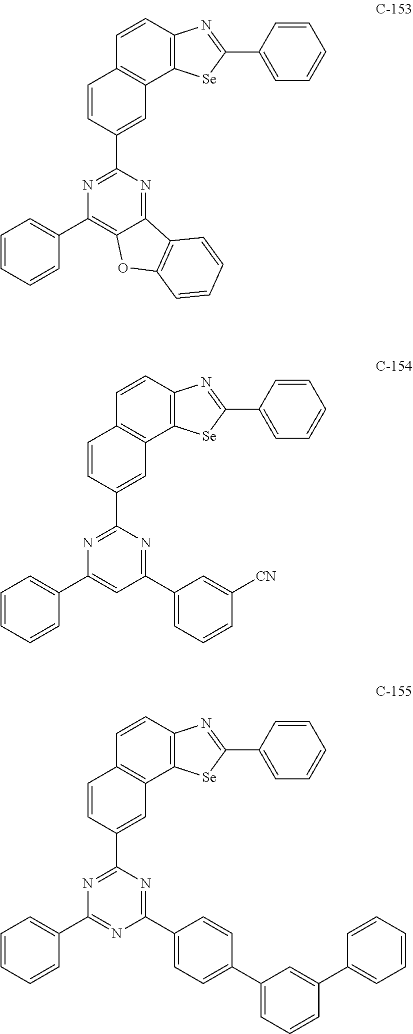 [SMILES: c1ccc(-c2nc3ccc4ccc(-c5nc(-c6ccccc6)c6oc7ccccc7c6n5)cc4c3[se]2)cc1 ; N#Cc1cccc(-c2cc(-c3ccccc3)nc(-c3ccc4ccc5nc(-c6ccccc6)[se]c5c4c3)n2)c1 ; c1ccc(-c2cccc(-c3ccc(-c4nc(-c5ccccc5)nc(-c5ccc6ccc7nc(-c8ccccc8)[se]c7c6c5)n4)cc3)c2)cc1]

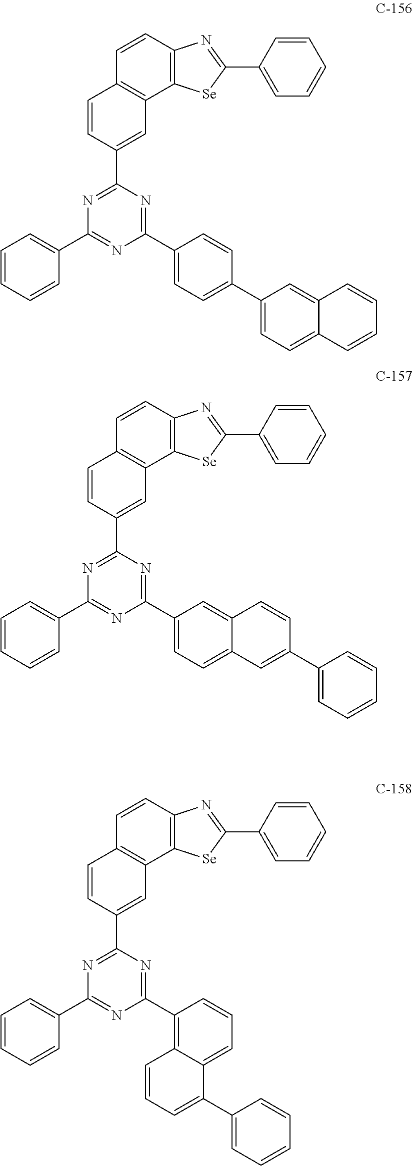 [SMILES: c1ccc(-c2nc(-c3ccc(-c4ccc5ccccc5c4)cc3)nc(-c3ccc4ccc5nc(-c6ccccc6)[se]c5c4c3)n2)cc1 ; c1ccc(-c2ccc3cc(-c4nc(-c5ccccc5)nc(-c5ccc6ccc7nc(-c8ccccc8)[se]c7c6c5)n4)ccc3c2)cc1 ; c1ccc(-c2nc(-c3ccc4ccc5nc(-c6ccccc6)[se]c5c4c3)nc(-c3cccc4c(-c5ccccc5)cccc34)n2)cc1]

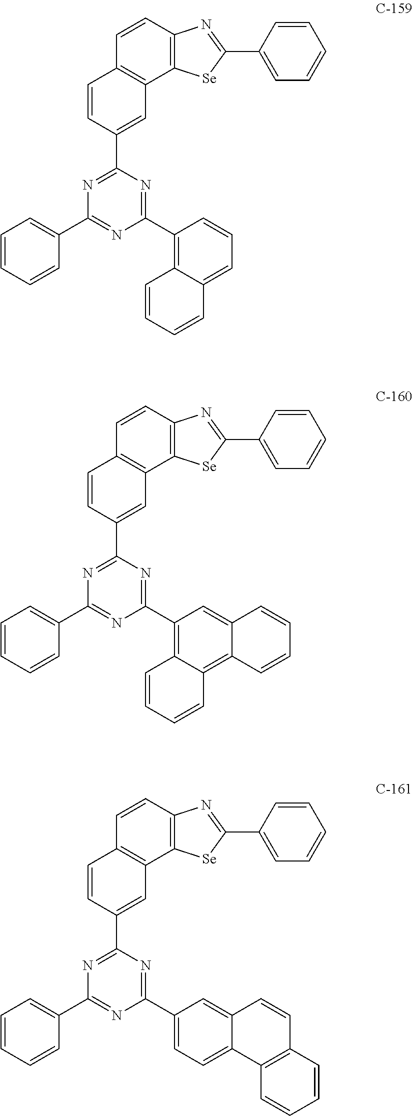 [SMILES: c1ccc(-c2nc(-c3ccc4ccc5nc(-c6ccccc6)[se]c5c4c3)nc(-c3cccc4ccccc34)n2)cc1 ; c1ccc(-c2nc(-c3ccc4ccc5nc(-c6ccccc6)[se]c5c4c3)nc(-c3cc4ccccc4c4ccccc34)n2)cc1 ; c1ccc(-c2nc(-c3ccc4c(ccc5ccccc54)c3)nc(-c3ccc4ccc5nc(-c6ccccc6)[se]c5c4c3)n2)cc1]

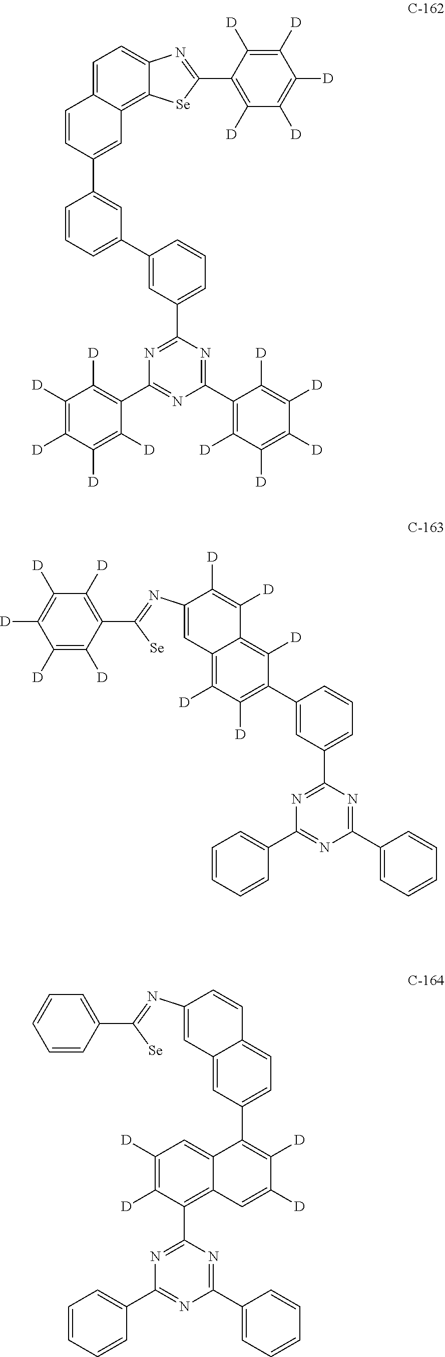

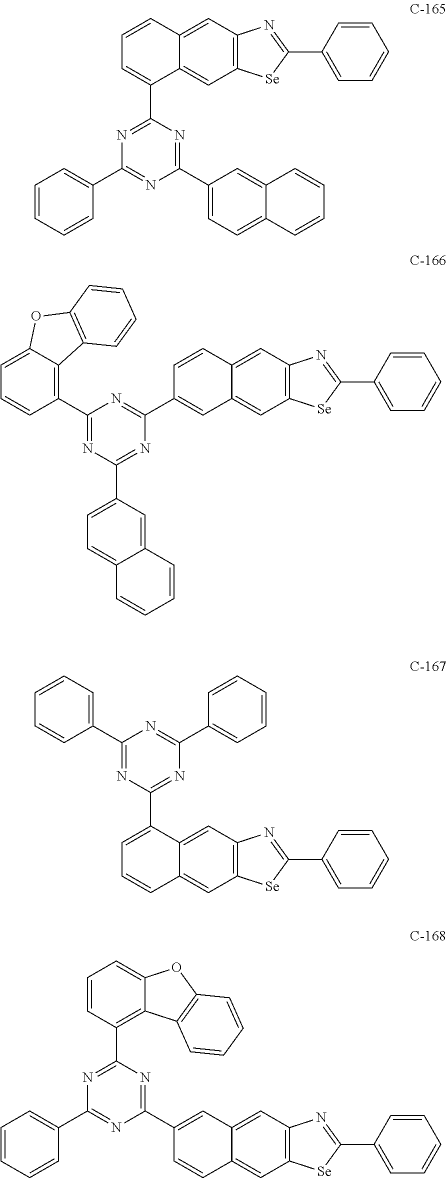 [SMILES: c1ccc(-c2nc(-c3ccc4ccccc4c3)nc(-c3cccc4cc5nc(-c6ccccc6)[se]c5cc34)n2)cc1 ; c1ccc(-c2nc3cc4ccc(-c5nc(-c6ccc7ccccc7c6)nc(-c6cccc7oc8ccccc8c67)n5)cc4cc3[se]2)cc1 ; c1ccc(-c2nc(-c3ccccc3)nc(-c3cccc4cc5[se]c(-c6ccccc6)nc5cc34)n2)cc1 ; c1ccc(-c2nc(-c3ccc4cc5[se]c(-c6ccccc6)nc5cc4c3)nc(-c3cccc4oc5ccccc5c34)n2)cc1]

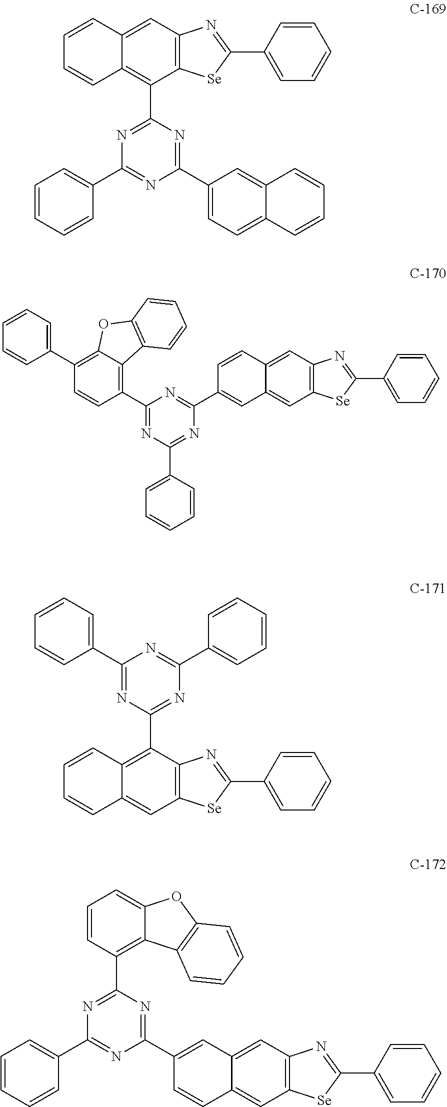 [SMILES: c1ccc(-c2nc(-c3ccc4ccccc4c3)nc(-c3c4ccccc4cc4nc(-c5ccccc5)[se]c34)n2)cc1 ; c1ccc(-c2nc(-c3ccc4cc5nc(-c6ccccc6)[se]c5cc4c3)nc(-c3ccc(-c4ccccc4)c4oc5ccccc5c34)n2)cc1 ; c1ccc(-c2nc(-c3ccccc3)nc(-c3c4ccccc4cc4[se]c(-c5ccccc5)nc34)n2)cc1 ; c1ccc(-c2nc(-c3ccc4cc5[se]c(-c6ccccc6)nc5cc4c3)nc(-c3cccc4oc5ccccc5c34)n2)cc1]

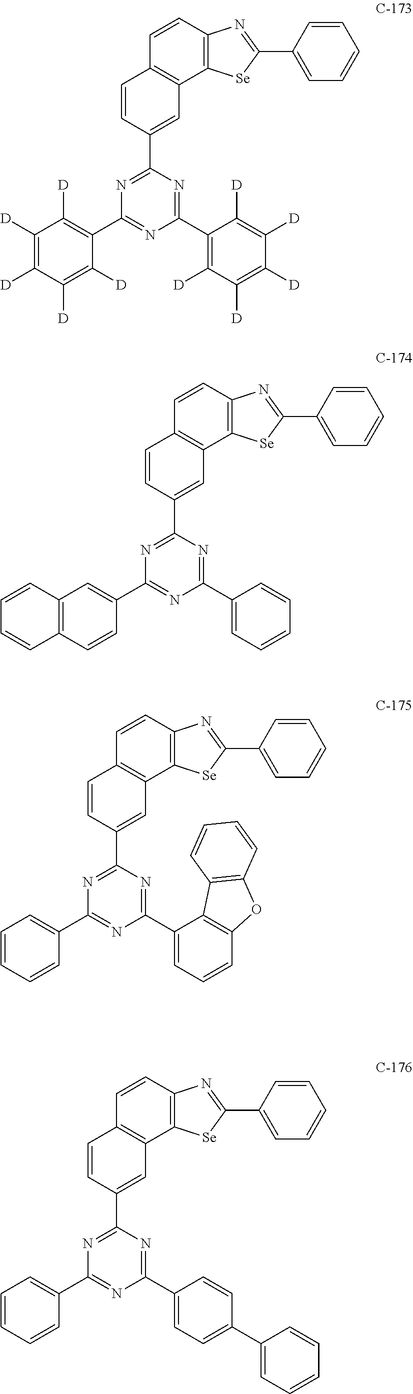 [SMILES: [2H]c1c([2H])c([2H])c(-c2nc(-c3ccc4ccc5nc(-c6ccccc6)[se]c5c4c3)nc(-c3c([2H])c([2H])c([2H])c([2H])c3[2H])n2)c([2H])c1[2H] ; c1ccc(-c2nc(-c3ccc4ccccc4c3)nc(-c3ccc4ccc5nc(-c6ccccc6)[se]c5c4c3)n2)cc1 ; c1ccc(-c2nc(-c3ccc4ccc5nc(-c6ccccc6)[se]c5c4c3)nc(-c3cccc4oc5ccccc5c34)n2)cc1 ; c1ccc(-c2ccc(-c3nc(-c4ccccc4)nc(-c4ccc5ccc6nc(-c7ccccc7)[se]c6c5c4)n3)cc2)cc1]

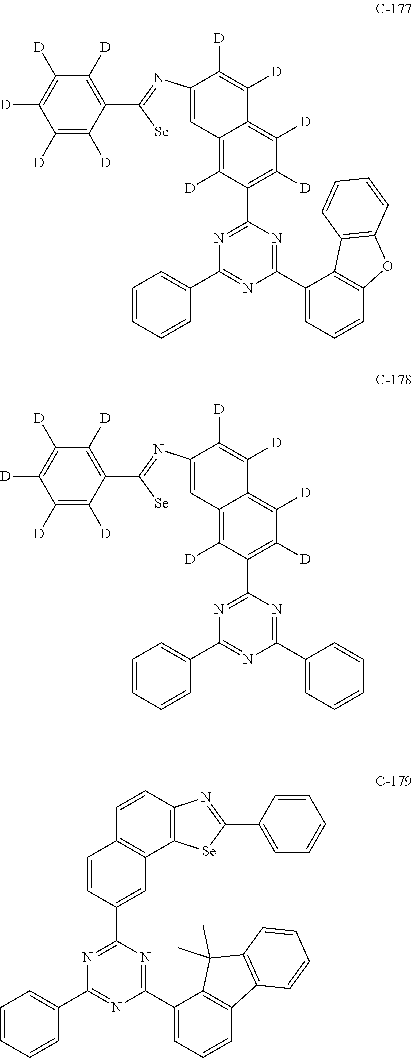

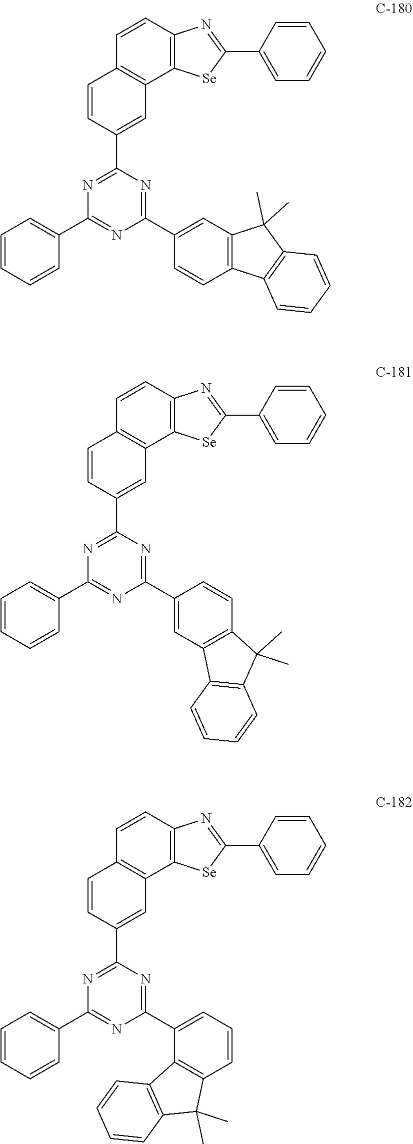 [SMILES: CC1(C)c2ccccc2-c2ccc(-c3nc(-c4ccccc4)nc(-c4ccc5ccc6nc(-c7ccccc7)[se]c6c5c4)n3)cc21 ; CC1(C)c2ccccc2-c2cc(-c3nc(-c4ccccc4)nc(-c4ccc5ccc6nc(-c7ccccc7)[se]c6c5c4)n3)ccc21 ; CC1(C)c2ccccc2-c2c(-c3nc(-c4ccccc4)nc(-c4ccc5ccc6nc(-c7ccccc7)[se]c6c5c4)n3)cccc21]

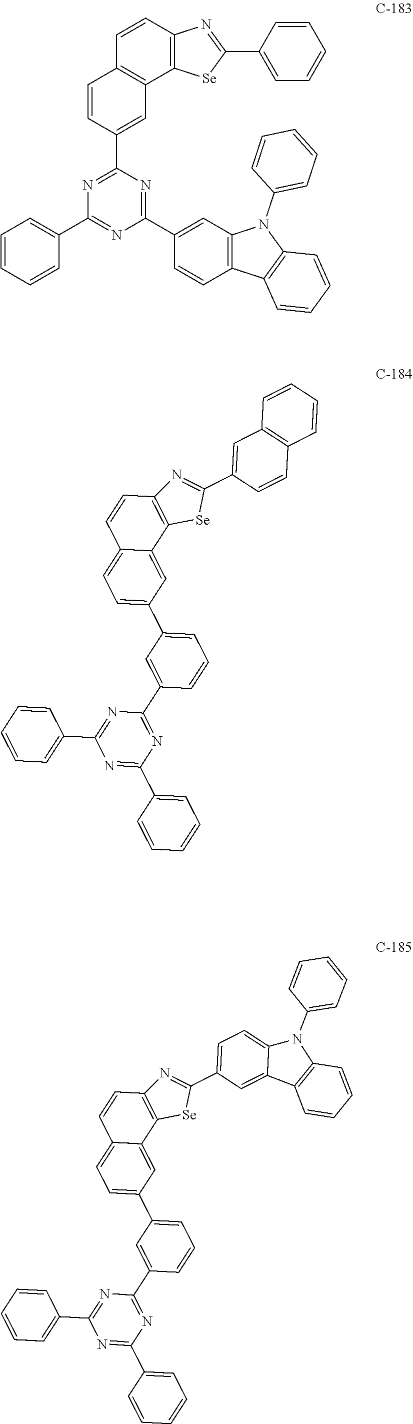 [SMILES: c1ccc(-c2nc(-c3ccc4ccc5nc(-c6ccccc6)[se]c5c4c3)nc(-c3ccc4c5ccccc5n(-c5ccccc5)c4c3)n2)cc1 ; c1ccc(-c2nc(-c3ccccc3)nc(-c3cccc(-c4ccc5ccc6nc(-c7ccc8ccccc8c7)[se]c6c5c4)c3)n2)cc1 ; c1ccc(-c2nc(-c3ccccc3)nc(-c3cccc(-c4ccc5ccc6nc(-c7ccc8c(c7)c7ccccc7n8-c7ccccc7)[se]c6c5c4)c3)n2)cc1]

The compound of formula 1 according to the present disclosure may be prepared as represented by the following reaction schemes 1 to 6, but is not limited thereto; they may further be produced by a synthetic method known to a person skilled in the art.

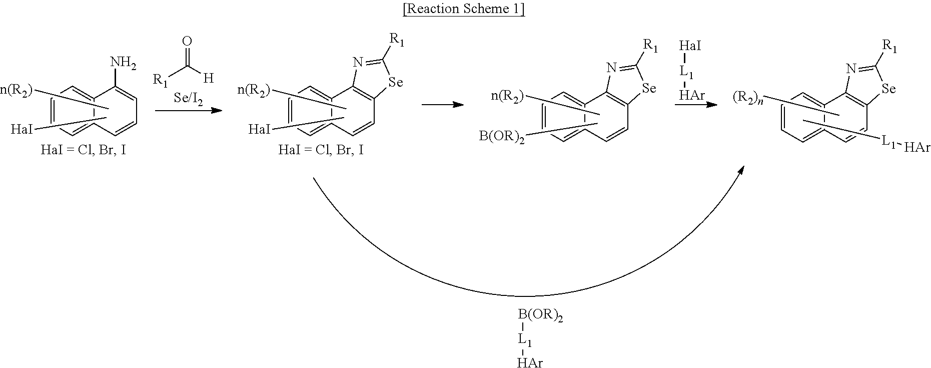

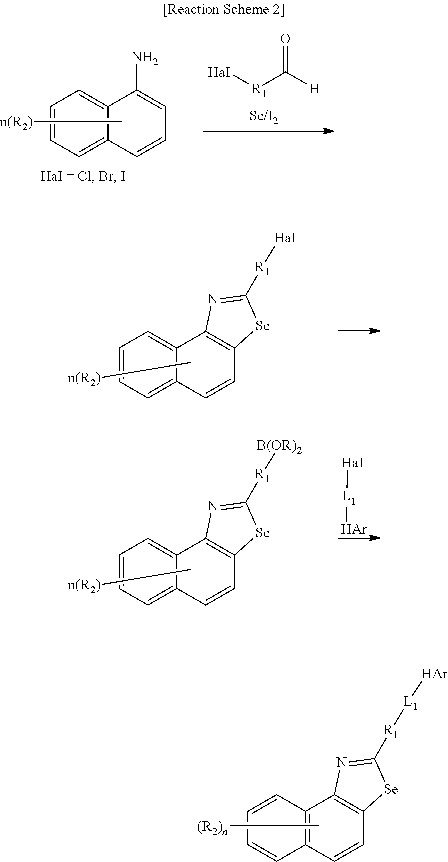

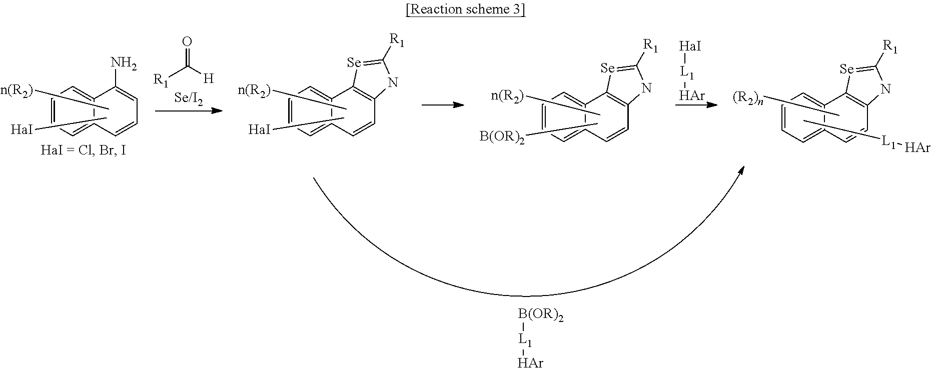

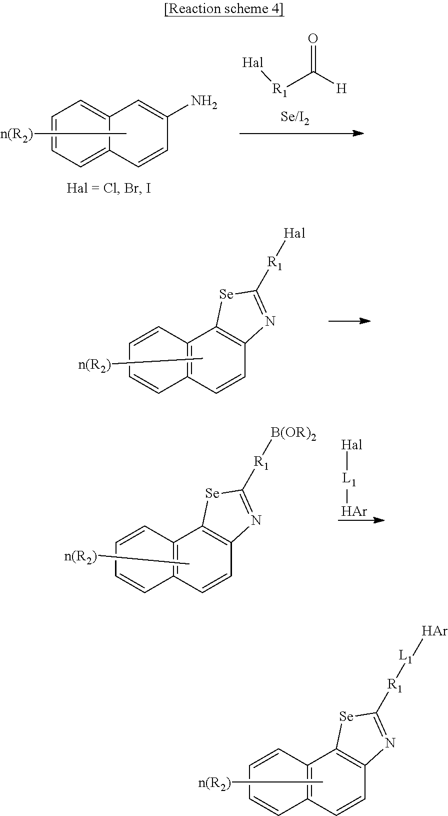

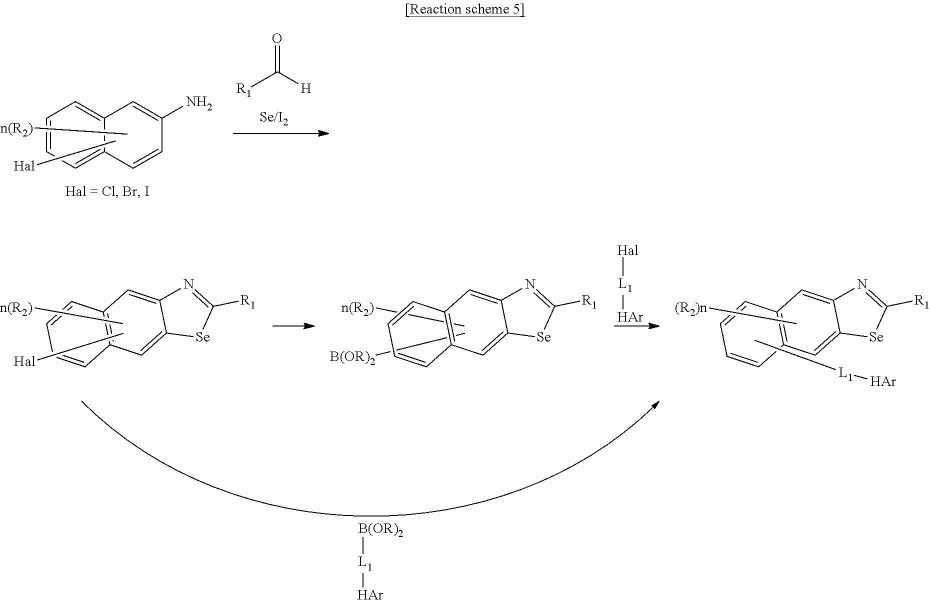

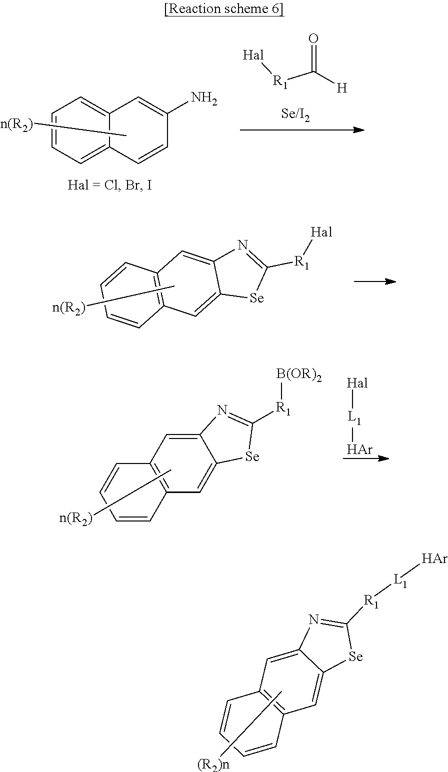

In reaction schemes 1 to 6, the definition of each of the substituents is as defined in formula 1.

As described above, exemplary synthesis examples of the compounds represented by formula 1 according to the present disclosure are described, but they are based on Sandmeyer reaction, Buchwald-Hartwig cross coupling reaction, N-arylation reaction, H-mont-mediated etherification reaction, Miyaura borylation reaction, Suzuki cross-coupling reaction, Intramolecular acid-induced cyclization reaction, Pd(II)-catalyzed oxidative cyclization reaction, Grignard reaction. Heck reaction. Cyclic Dehydration reaction, SN₁ substitution reaction, SN₂ substitution reaction, and Phosphine-mediated reductive cyclization reaction, etc. It will be understood by one skilled in the art that the above reaction proceeds even if other substituents defined in the formula 1 other than the substituents described in the specific synthesis examples are bonded.

According to one embodiment, the present disclosure may provide an organic electroluminescent material comprising an organic electroluminescent compound of formula 1, and an organic electroluminescent device comprising an organic electroluminescent material.

The organic electroluminescent material may consist of the organic electroluminescent compound of the present disclosure alone, or may further include conventional materials included in the organic electroluminescent material. When two or more materials are included in one layer, they may be mixed and deposited to form a layer, or may be separately and simultaneously co-deposited to form a layer. The organic electroluminescent material according to one embodiment may comprise at least one compound(s) represented by formula 1 above. The organic electroluminescent compound of formula 1 of the present disclosure may preferably be included in the hole transport layer, the light-emitting layer, the buffer layer, and/or the electron transport layer of the organic electroluminescent device, more preferably, the electron transport layer or the light emitting layer. When included in the light-emitting layer, the organic electroluminescent compound of formula 1 may be included as a host, and more specifically, may be included as a phosphorescent red host.

According to one embodiment, the present disclosure provides a plurality of host materials comprising a first host material represented by formula 1 and a second host material which is different from the first host material. Specifically, the second host material according to one embodiment comprises at least one compound(s) of organic electroluminescent compounds represented by the following formulas 2 to 5.

The second host material according to one embodiment comprises an organic electroluminescent compound of formula 2.

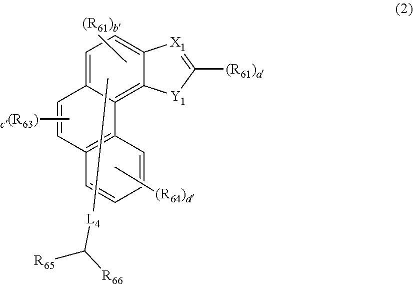

in formula 2.

X₁ and Y₁ each independently represent —N═, —NR₆₇—, —O— or —S—; provided that, one of X₁ and Y₁ is —N═, and the other of X₁ and Y₁ is —NR₆₇—. —O— or —S—;

R₆₁ represents a substituted or unsubstituted (C6-C30)aryl or a substituted or unsubstituted (3- to 30-membered)heteroaryl;

R₆₂ to R₆₄ and R₆₇ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsiyl, a substituted or unsubstituted tri(C6-C30)arylsilyl, a substituted or unsubstituted fused ring of (C3-C30) aliphatic ring and an (C6-C30) aromatic ring, or -L₃″-N(Ar₃″)(Ar₄″); or may be linked to the adjacent substituents to form a ring(s);

L₃″ represents a single bond, a substituted or unsubstituted (C6-C30)arylene, or a substituted or unsubstituted (3- to 30-membered)heteroarylene;

Ar₃″ and Ar₄″ each independently represent hydrogen, a substituted or unsubstituted (C1-C30)alkyl, a substituted or unsubstituted (C2-C30)alkenyl, a substituted or unsubstituted fused ring of (C3-C30) aliphatic ring and an (C6-C30) aromatic ring, a substituted or unsubstituted (C6-C30)aryl, or a substituted or unsubstituted (3- to 30-membered)heteroaryl;

L₄ represents a single bond, a substituted or unsubstituted (C6-C30)arylene, or a substituted or unsubstituted (3- to 30-membered)heteroarylene;

R₆₅ and R₆₆ each independently represent a substituted or unsubstituted (C6-C30)aryl, or a substituted or unsubstituted (3- to 30-membered)heteroaryl;

a′ is 1, b′ and c′ each independently represent 1 or 2, and d′ represents an integer of 1 to 4; and

when b′ to d′ are 2 or more, each of R₆₂ to R₆₄ may be the same or different.

In one embodiment, in formula 2, one of X₁ and Y₁ is —N═, and the other X₁ and Y₁ may be —O— or —S—. For example, X₁ is —N═ and Y₁ is —O—, or X₁ is —O— and Y₁ is —N═, or X₁ is —S— and Y₁ is —N═.

In one embodiment, in formula 2, R₆₁ may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C18)aryl or a substituted or unsubstituted (5- to 18-membered)heteroaryl. For example, R₆₁ may be an unsubstituted phenyl, an unsubstituted naphthyl, an unsubstituted o-biphenyl, an unsubstituted m-biphenyl, an unsubstituted p-biphenyl, or an unsubstituted pyridyl.

In one embodiment, in formula 2, R₆₂ to R₆₄ and R₆₇ each independently may be hydrogen, deuterium, halogen, cyano, a substituted or unsubstituted (C1-C30)alkyl, or a substituted or unsubstituted (C6-C30)aryl, preferably hydrogen, deuterium, halogen, cyano, or a substituted or unsubstituted (C6-C25)aryl, more preferably hydrogen, deuterium, or a substituted or unsubstituted (C6-C18)aryl. For example, R₆₂ to R₆₄ each independently may be hydrogen or an unsubstituted phenyl.

In one embodiment, in formula 2, L₄ may be a single bond or a substituted or unsubstituted (C6-C30)arylene, preferably a single bond or a substituted or unsubstituted (C6-C25)arylene, more preferably a substituted or unsubstituted (C6-C18)arylene. For example, L₄ may be a single bond or an unsubstituted phenylene or an unsubstituted naphthylene.

In one embodiment, in formula 2, R₆₅ and R₆₆ each independently may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably a substituted or unsubstituted (C6-C25)aryl or a substituted or unsubstituted (5- to 20-membered)heteroaryl. For example, R₆₅ and R₆₆ each independently may be a substituted or unsubstituted phenyl, a substituted or unsubstituted o-biphenyl, a substituted or unsubstituted m-biphenyl, a substituted or unsubstituted p-biphenyl, a substituted or unsubstituted naphthyl, a substituted or unsubstituted m-terphenyl, a substituted or unsubstituted o-terphenyl, a substituted or unsubstituted fluorenyl, a substituted or unsubstituted phenanthrenyl, a substituted or unsubstituted benzofuranyl, a substituted or unsubstituted dibenzofuranyl, a substituted or unsubstituted dibenzothiophenyl, a substituted or unsubstituted benzofluorenyl, a substituted or unsubstituted spirobifluorenyl, a substituted or unsubstituted C22 aryl, a substituted or unsubstituted carbazolyl, a substituted or unsubstituted benzonaphthofuranyl, or a substituted or unsubstituted benzofuropyridyl. The substituent of the substituents may be at least one of phenyl unsubstituted or substituted with at least one of deuterium; methyl; tert-butyl; cyclohexyl; methyl and tert-butyl; naphthyl; biphenyl; anthracenyl; fluoranthenyl; phenylfluorenyl; pyridyl unsubstituted or substituted with phenyl; phenoxazinyl; diphenylamino; benzimidazolyl substituted with phenyl; triphenylsilanyl; diphenylnaphthylsilanyl; and biphenyldiphenylsilanyl.

The second host material according to one embodiment comprises an organic electroluminescent compound of formula 3.

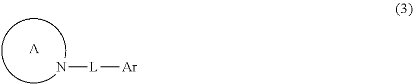

in formula 3,

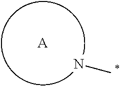

is a structure in which 5 to 8 substituted or unsubstituted (C6-C30)arene rings or a substituted or unsubstituted (3- to 30-membered)heteroarene ring are fused around a 7-membered or 8-membered ring, wherein,

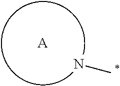

includes at least one pentacyclic ring(s) containing at least one nitrogen(s);

L represents a single bond, a substituted or unsubstituted (C1-C30)alkylene, a substituted or unsubstituted (C3-C30)cycloalkylene, a substituted or unsubstituted (C6-C30)arylene, or a substituted or unsubstituted (3- to 30-membered)heteroarylene;

Ar represents deuterium, halogen, cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₁R₁₂ or —SiR₁₃R₁₄R₁₅; and

R₁₁ to R₁₅ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

In one embodiment, in formula 3, L may be a single bond, a substituted or unsubstituted (C6-C30)arylene, or a substituted or unsubstituted (5- to 30-membered)heteroarylene, preferably a single bond, a substituted or unsubstituted (C6-C25)arylene, or a substituted or unsubstituted (5- to 20-membered)heteroarylene, more preferably a single bond, an unsubstituted (C6-C18)arylene, or (5- to 20-membered)heteroarylene unsubstituted or substituted with (C6-C15)aryl. For example, L may be a single bond, phenylene, naphthylene, biphenylene, pyridylene, pyrimidinylene, triazinylene, quinolinylene, quinazolinylene, quinoxalinylene, naphthyridinylene, carbazolylene, dibenzofuranylene, benzofuropyrimidinylene, benzothienopyrimidinylene, pyrimidoindolylene, benzoquinazolinylene, benzoquinoxalinylene, phenylquinazolinylene, or phenylquinoxalinylene, etc.

In one embodiment, in formula 3, Ar may be deuterium, halogen, cyano, a substituted or unsubstituted (C1-C30)alkyl, a substituted or unsubstituted (C5-C30)cycloalkyl, a substituted or unsubstituted (C5-C30)cycloalkenyl, a substituted or unsubstituted (3- to 7-membered)heterocycloalkyl, a substituted or unsubstituted (C6-C30)aryl, a substituted or unsubstituted (5- to 30-membered)heteroaryl, —NR₁₁R₁₂ or —SiR₁₃R₁₄R₁₅, preferably a substituted or unsubstituted (C6-C25)aryl, a substituted or unsubstituted (5- to 20-membered)heteroaryl, or —NR₁₁R₁₂, more preferably (C6-C25)aryl unsubstituted or substituted with (C1-C6)alkyl; (5- to 20-membered)heteroaryl unsubstituted or substituted with at least one of deuterium, (C1-C6)alkyl, (C6-C18)aryl and (5- to 26-membered)heteroaryl; or —NR₁₁R₁₂. For example. Ar may be an unsubstituted phenyl, phenyl substituted with at least one deuterium(s), phenyl substituted with 26-membered heteroaryl, naphthyl, biphenyl, fluorenyl substituted with at least one methyl, benzofluorenyl substituted with at least one methyl, spirobifluorenyl, terphenyl, triphenylenyl, pyridyl unsubstituted or substituted with phenyl, pyrimidinyl substituted with phenyl, a substituted triazinyl, a substituted quinoxalinyl, a substituted quinazolinyl, quinolyl substituted with phenyl, naphthyridinyl substituted with phenyl, benzoquinazolinyl substituted with phenyl, benzoquinoxalinyl substituted with phenyl, carbazolyl unsubstituted or substituted with phenyl, dibenzofuranyl unsubstituted or substituted with phenyl, dibenzothiophenyl unsubstituted or substituted with phenyl, benzofuropyrimidinyl substituted with phenyl, benzothienopyrimidinyl substituted with phenyl, pyrimidoindolyl substituted with phenyl, or —NR₁₁R₁₂. The substituent of the substituted triazinyl, the substituted quinoxalinyl, and the substituted quinazolinyl each independently may be at least one of phenyl unsubstituted or substituted with at least one of deuterium and 26-membered heteroaryl, naphthyl, biphenyl, terphenyl, dimethylfluorenyl, pyridyl unsubstituted or substituted with phenyl, dibenzofuranyl and dibenzothiophenyl.

In one embodiment, in formula 3, R₁₁ to R₁₅ each independently may be a substituted or unsubstituted (C1-C30)alkyl, a substituted or unsubstituted (C6-C30)aryl, or a substituted or unsubstituted (5- to 30-membered)heteroaryl, preferably, a substituted or unsubstituted (C6-C15)aryl or a substituted or unsubstituted (5- to 25-membered)heteroaryl, more preferably, (C6-C18)aryl unsubstituted or substituted with at least one (C1-C6)alkyl, or (5- to 20-membered)heteroaryl unsubstituted or substituted with (C6-C18)aryl. For example, R₁₁ to R₁₅ each independently may be phenyl, naphthyl, biphenyl, or dimethylfluorenyl, etc.

According to one embodiment,

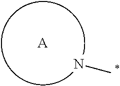

may be represented by any one of the following formulas 3-1 to 3-3.

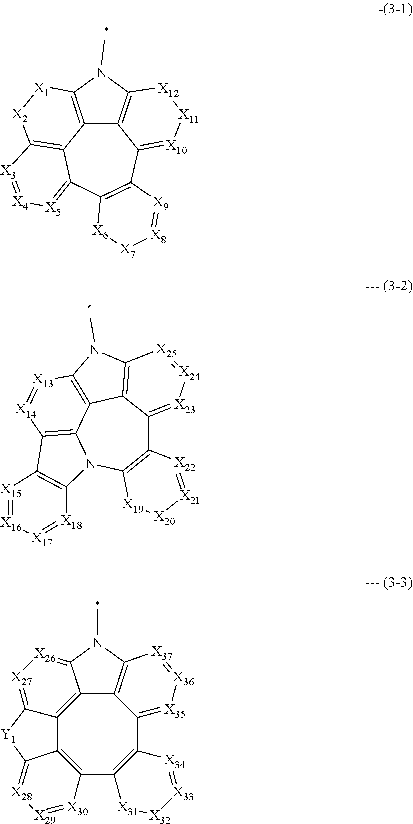

in formulas 3-1 to 3-3,

X₁ to X₃₇ each independently represent —N═ or —C(R₁₁)═;

R₁₁ represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or adjacent Rₐs may be linked to each other to form a ring(s);

Y₁ represents —N(L₁₁-(Ar₁₁)ₘ)—, —O—, —S—, or —C(R₁₂)(R₁₃)—;

L₁₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

Ar₁₁ represents a substituted or unsubstituted (C6-C30)aryl, a substituted or unsubstituted (3- to 30-membered)heteroaryl, or —N(R₁₄)(R₁₅);

R₁₂ to R₁₅ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to each other to form a ring(s); and

m is an integer of 1 or 2; and

when m is 2, each of Ar₁₁ may be the same or different.

In one embodiment, all of X₁ to X₃₇ may be —C(R₁₁)═.

In one embodiment, Rn may be hydrogen, a substituted or unsubstituted (C6-C25)aryl, or a substituted or unsubstituted (5- to 25-membered)heteroaryl; or adjacent R₁₁ may be linked to each other to form a substituted or unsubstituted (3- to 30-membered) mono- or polycyclic, alicyclic, aromatic ring, or a combination thereof, preferably hydrogen, an unsubstituted (C6-C18)aryl, or (5- to 20-membered)heteroaryl substituted with at least one (C6-C18)aryl; or adjacent R₁₈ may be linked to each other to form a substituted or unsubstituted (5- to 20-membered) mono- or polycyclic, alicyclic, aromatic ring, or a combination thereof. For example, R₁₁ may be hydrogen, phenyl, naphthyl, or triazinyl substituted with at least one phenyl; or adjacent R₁₁ may be linked to each other to form benzene ring, benzofuran ring, or indene ring substituted with at least methyl.

In one embodiment, in formula 3-3, Y₁ may be —N(L₁₁-(Ar₁₁)ₘ)—, —O—, —S—, or —C(R₁₂)(R₁₃)—.

In one embodiment, L₁₁ may be a single bond or a substituted or unsubstituted (C6-C30)arylene. For example, L′₁ may be a single bond or phenylene.

In one embodiment, Ar₁₁ may be a substituted or unsubstituted (C6-C30)aryl or a substituted or unsubstituted (5- to 30-membered)heteroaryl, preferably a substituted or unsubstituted (C6-C25)aryl or a substituted or unsubstituted (5- to 25-membered)heteroaryl, more preferably, (C6-C18)aryl unsubstituted or substituted with deuterium or an unsubstituted (5- to 18-membered)heteroaryl. For example, Ar₁₁ may be phenyl unsubstituted or substituted with deuterium, biphenyl, or pyridyl.

In one embodiment, R₁₂ and R₁₃ each independently may be a substituted or unsubstituted (C1-C10)alkyl. For example, R₁₂ and R₁₃ each independently may be methyl.

The formula 3-1 according to one embodiment may be represented by the following formula 3-1-1.

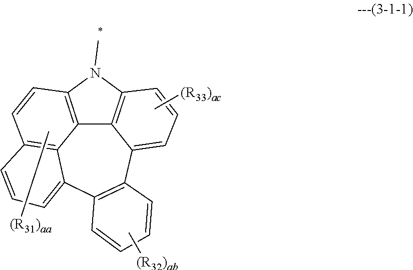

in formula 3-1-1,

R₃₁ to R₃₃ are each independently as defined as R₁₁ in formula 3-1;

aa is an integer of 1 to 5, ab is an integer of 1 to 4, and ac is an integer of 1 to 3; and

when aa, ab, and ac are 2 or more, each of R₃₁, each of R₃₂ and each of R₃₃ may be the same or different.

The formula 3-2 according to one embodiment may be represented by the following formula 3-2-1.

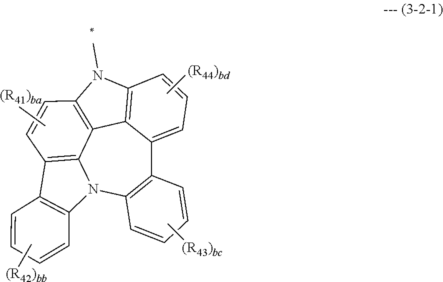

in formula 3-2-1,

R₄₁ to R₄₄ are each independently as defined as R₁₁ in formula 3-2;

ba is an integer of 1 or 2, bb and bc are each independently an integer of 1 to 4, and bd is an integer of 1 to 3; and

when ba, bb, bc, and bd are 2 or more, each of R₄₁, each of R₄₂, each of R₄₃ and each of R₄₄ may be the same or different.

The formula 3-3 according to one embodiment may be represented by the following formula 3-3-1.

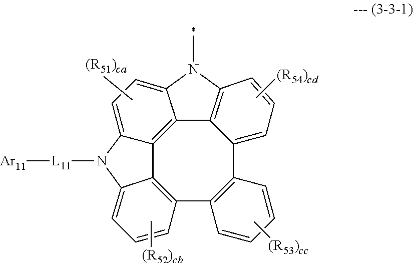

in formula 3-3-1,

R₅₁ to R₅₄ are each independently as defined as R₁₁ in formula 3-3;

L₁₁ and Ar₁₁ are as defined in formula 3-3;

ca is an integer of 1 or 2, cb and cd are each independently an integer of 1 to 3, and cc is an integer of 1 to 4; and

when ca, cb, cc, and cd are 2 or more, each of R₅₁, each of R₅₂, each of R₅₃ and each of R₅₄ may be the same or different.

The second host material according to one embodiment comprises an organic electroluminescent compound of formula 4.

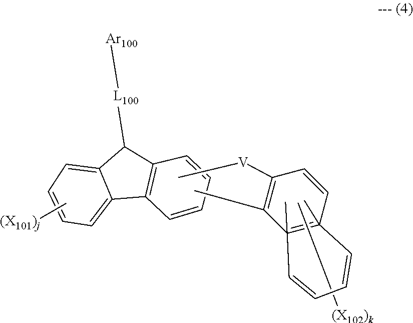

in formula 4,

V represents CX₁₁X₁₂, NX₁₃, O, or S;

L₁₀₀ represents a single bond, a substituted or unsubstituted (C6-C30)arylene, or a substituted or unsubstituted (3- to 30-membered)heteroarylene;

Ar₁₀₀ represents a substituted or unsubstituted (C6-C30)aryl, a substituted or unsubstituted (3- to 30-membered)heteroaryl, or —NX₉X₁₀;

X₉ and X₁₀ each independently represent a substituted or unsubstituted (C1-C30)alkyl, a substituted or unsubstituted (C3-C30)cycloalkyl, a substituted or unsubstituted (C6-C30)aryl, or a substituted or unsubstituted (3- to 30-membered)heteroaryl;

X₁₁ to X₁₃, X₁₀₁, and X₁₀₂ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted (C2-C30)alkenyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(3- to 30-membered)heteroarylamino, or a substituted or unsubstituted (C6-C30)aryl(3- to 30-membered)heteroarylamino;

j is an integer of 1 to 4, and k is an integer of 1 to 6; and

when j and k are 2 or more, each of X₁₀₁ and each of X₁₀₂ may be the same or different.

In one embodiment, in formula 4, V may be NX₁₃, wherein X₁₃ may be a substituted or unsubstituted (C6-C30)aryl, preferably, a substituted or unsubstituted (C6-C25)aryl, more preferably, a substituted or unsubstituted (C6-C18)aryl. For example, X₁₃ may be an unsubstituted phenyl.

In one embodiment, in formula 4, L₁₀₀ may be a single bond, a substituted or unsubstituted (C6-C30)arylene, or a substituted or unsubstituted (5- to 30-membered)heteroarylene, preferably, a single bond, a substituted or unsubstituted (C6-C25)arylene, or a substituted or unsubstituted (5- to 25-membered)heteroarylene, more preferably, a single bond, a substituted or unsubstituted (C6-C18)arylene, or a substituted or unsubstituted (5- to 18-membered)heteroarylene. For example, L₁₀₀ may be a single bond, a substituted or unsubstituted phenylene, a substituted or unsubstituted biphenylene, a substituted or unsubstituted pyridylphenylene, a substituted or unsubstituted naphthalenylene, a substituted or unsubstituted quinolinylene, a substituted or unsubstituted quinazolinylene, a substituted or unsubstituted quinoxalinylene, a substituted or unsubstituted carbazolylene, a substituted or unsubstituted naphthyridinylene, a substituted or unsubstituted benzoquinoxalinylene, a substituted or unsubstituted benzoquinazolinylene, or a substituted or unsubstituted benzofuropyrimidinylene.

In one embodiment, in formula 4, Ar₁₀₀ may be a substituted or unsubstituted (C6-C30)aryl, a substituted or unsubstituted (5- to 30-membered)heteroaryl, or —NX₉X₁₀, preferably, a substituted or unsubstituted (C6-C25)aryl, a substituted or unsubstituted (5- to 25-membered)heteroaryl, or —NX₉X₁₀, more preferably, a substituted or unsubstituted (C6-C18)aryl, a substituted or unsubstituted (5- to 18-membered)heteroaryl, or —NX₉X₁₀. Wherein, X₉ and X₁₀ each independently may be a substituted or unsubstituted (C6-C30)aryl, preferably, a substituted or unsubstituted (C6-C25)aryl, more preferably, a substituted or unsubstituted (C6-C18)aryl. For example, Ar₁₀₀ may be a substituted or unsubstituted phenyl, a substituted or unsubstituted naphthyl, a substituted or unsubstituted m-biphenyl, a substituted or unsubstituted p-biphenyl, a substituted or unsubstituted o-terphenyl, a substituted or unsubstituted m-terphenyl, a substituted or unsubstituted p-terphenyl, a substituted or unsubstituted dibenzofuranyl, a substituted or unsubstituted dibenzothiophenyl, a substituted or unsubstituted carbazolyl, a substituted or unsubstituted fluorenyl, a substituted or unsubstituted benzofluorenyl, a substituted or unsubstituted spirobifluorenyl, a substituted or unsubstituted pyridyl, a substituted or unsubstituted pyrimidinyl, a substituted or unsubstituted naphthyridinyl, a substituted or unsubstituted triazinyl, a substituted or unsubstituted benzofuropyrimidinyl, phenylbiphenylamino, phenylnaphthylamino, or diphenylamino.

In one embodiment, in formula 4, X₁₀₁ and X₁₀₂ each independently may be hydrogen or deuterium.

The second host material according to one embodiment comprises an organic electroluminescent compound of formula 5.

T-Lₚ-Arₚ (5)

in formula 5,

Arₚ represents a substituted or unsubstituted (C6-C30)aryl, a substituted or unsubstituted (3- to 30-membered)heteroaryl, or

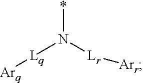

Ar_{q} and Arᵣ each independently represent a substituted or unsubstituted (C6-C30)aryl, a substituted or unsubstituted (3- to 30-membered)heteroaryl, or a substituted or unsubstituted (C3-C30)cycloalkyl;

Lₚ to Lᵣ each independently represent a single bond, a substituted or unsubstituted (C6-C30)arylene, or a substituted or unsubstituted (3- to 30-membered)heteroarylene; and

T is represented by the following formula 5-1 or 5-2;

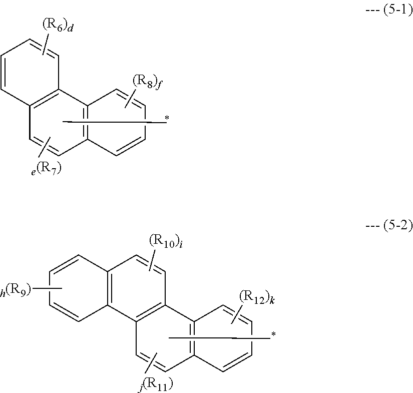

in formulas 5-1 and 5-2,

R₆ to R₁₂ each independently represent hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and an (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;

d, f, h, and k are each independently an integer of 1 to 4, and e, i, and j are each independently an integer of 1 or 2; and

when d to f and h to k are 2 or more, each of R₆ to R₁₁ may be the same or different.

In one embodiment, in formula 5, Re to R₁₂ each independently may be hydrogen, deuterium, a substituted or unsubstituted (C6-C25)aryl, a substituted or unsubstituted (5- to 25-membered)heteroaryl, or a substituted or unsubstituted mono- or di-(C6-C25)arylamino, preferably, hydrogen, deuterium, (C6-C28)aryl unsubstituted or substituted with (C6-C18)aryl, an unsubstituted (5- to 20-membered)heteroaryl, or an unsubstituted di(C6-C18)arylamino. For example, Re to R₁₂ each independently may be hydrogen, deuterium, phenyl, naphthyl, phenylnaphthyl, naphthylphenyl, phenanthrenyl, terphenyl, triphenylenyl, pyridyl, quinolyl, dibenzofuranyl, dibenzothiophenyl, diphenylamino, etc.

In one embodiment, in formula 5, Arₚ may be a substituted or unsubstituted (C6-C30)aryl, a substituted or unsubstituted (5- to 20-membered)heteroaryl, or

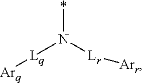

preferably, (C6-C30)aryl unsubstituted or substituted with at least one of (C1-C10)alkyl and (C6-C18)aryl; an unsubstituted (5- to 20-membered)heteroaryl, or

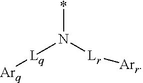

For example, Arₚ may be phenyl, naphthyl, biphenyl, phenanthrenyl, triphenylenyl, fluoranthenyl, terphenyl, dimethylfluorenyl, diphenylfluorenyl, dimethylbenzofluorenyl, diphenylbenzofluorenyl, spirobifluorenyl, spiro[cyclocyclopentan-fluoren]yl, spiro[dihydroinden-fluoren]yl, spiro[fluoren-benzofluoren]yl, dibenzofuranyl, dibenzothiophenyl, benzonaphthofuranyl, benzonaphthothiophenyl, etc.

In one embodiment, in formula 5. Ar_{q} and Arᵣ each independently may be a substituted or unsubstituted (C6-C25)aryl, or a substituted or unsubstituted (5- to 25-membered)heteroaryl, preferably, (C6-C25)aryl unsubstituted or substituted with at least one of deuterium, (C1-C10)alkyl, (5- to 20-membered)heteroaryl and tri(C6-C18)arylsilyl; or (5- to 20-membered)heteroaryl unsubstituted or substituted with (C6-C18)aryl. For example, Ar_{q} and Arᵣ each independently may be phenyl unsubstituted or substituted with tert-butyl, pyridyl or triphenylsilyl; naphthyl; naphthylphenyl; phenylnaphthyl; biphenyl unsubstituted or substituted with deuterium or tert-butyl; phenanthrenyl; terphenyl; dimethylfluorenyl unsubstituted or substituted with phenyl; diphenylfluorenyl; spirobifluorenyl; pyridyl unsubstituted or substituted with phenyl; benzofuranyl substituted with phenyl, dibenzofuranyl unsubstituted or substituted with phenyl; dibenzothiophenyl unsubstituted or substituted with phenyl; benzonaphthofuranyl; benzonaphthothiophenyl, etc.

In one embodiment, in formula 5, Lₚ to Lᵣ each independently may be a single bond, a substituted or unsubstituted (C6-C25)arylene, or a substituted or unsubstituted (5- to 25-membered)heteroarylene, preferably a single bond, (C6-C18)arylene unsubstituted or substituted with (C6-C18)aryl, or an unsubstituted (5- to 20-membered)heteroarylene. For example, Lₚ may be a single bond; phenylene unsubstituted or substituted with phenyl; naphthylene; biphenylene; pyridylene, etc., and L_{q} and Lᵣ each independently may be a single bond, phenylene, naphthylene, dibenzofluorenylene, etc.

The formula 5 according to one embodiment may be represented by the following formula 5-1-1 or 5-2-1.

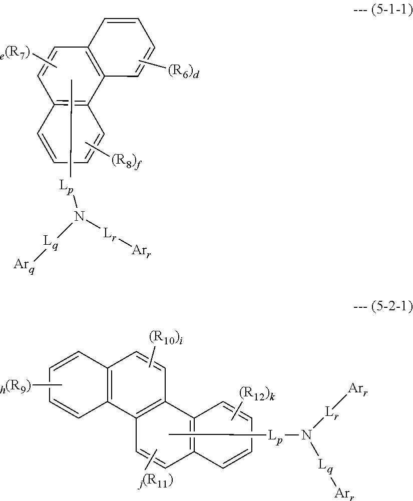

in formulas 5-1-1 and 5-2-1,

R₆ to R₁₂, Lₚ to Lᵣ, Ar_{q}, Arᵣ, d to f, and h to k are as defined in formula 5.

According to one embodiment, the organic electroluminescent compound represented by formula 2 may be more specifically illustrated by the following compounds, but is not limited thereto.

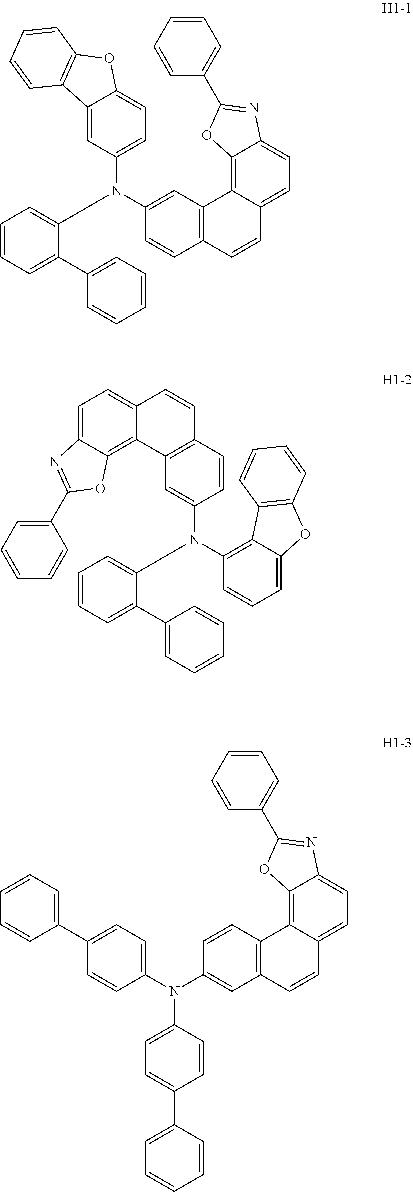 [SMILES: c1ccc(-c2nc3ccc4ccc5ccc(N(c6ccc7oc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

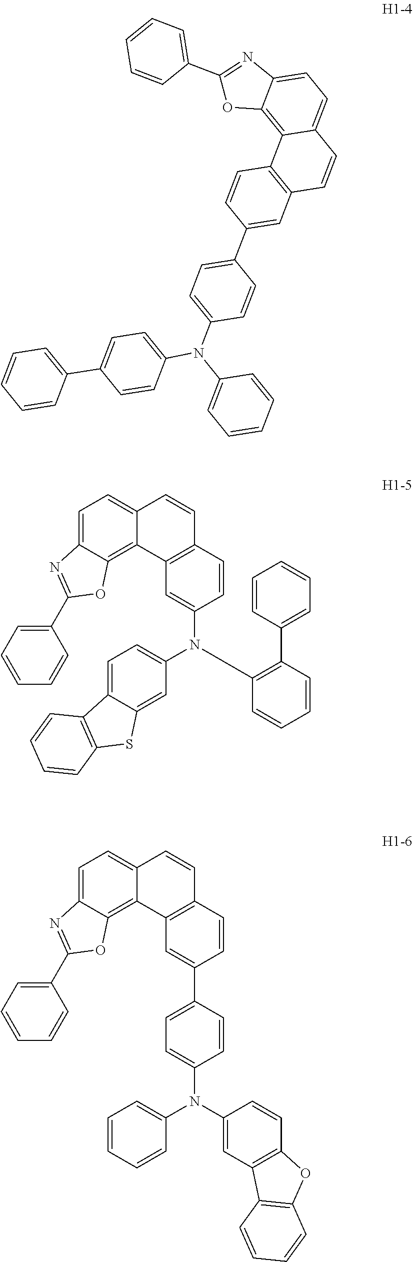 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

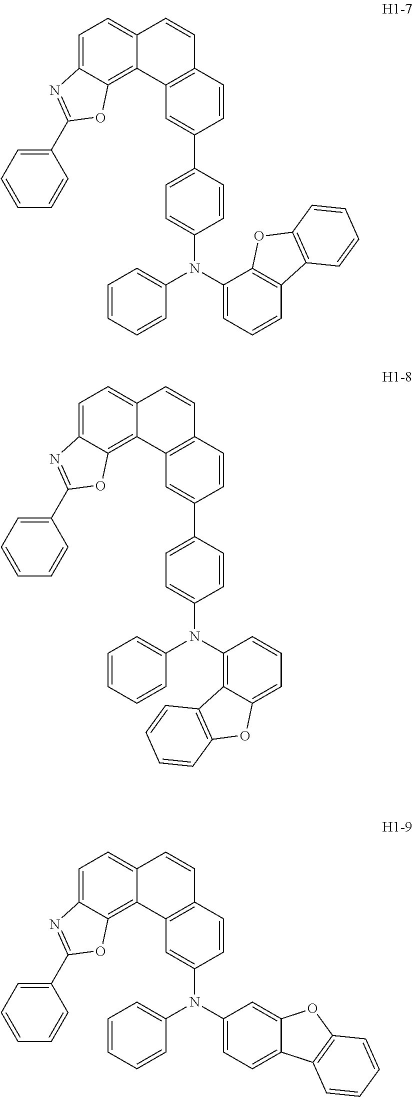 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccccc6)c6ccc7c(c6)oc6ccccc67)cc5c4c3o2)cc1]

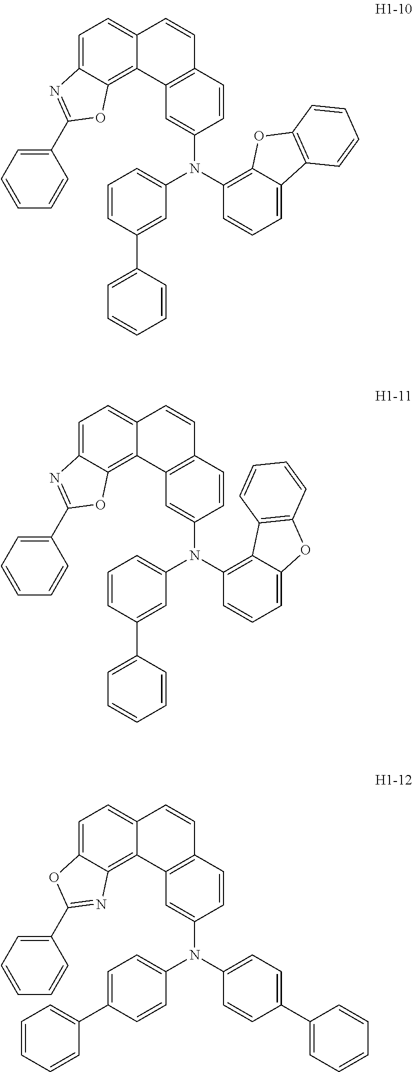 [SMILES: c1ccc(-c2cccc(N(c3ccc4ccc5ccc6nc(-c7ccccc7)oc6c5c4c3)c3cccc4c3oc3ccccc34)c2)cc1 ; c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1]

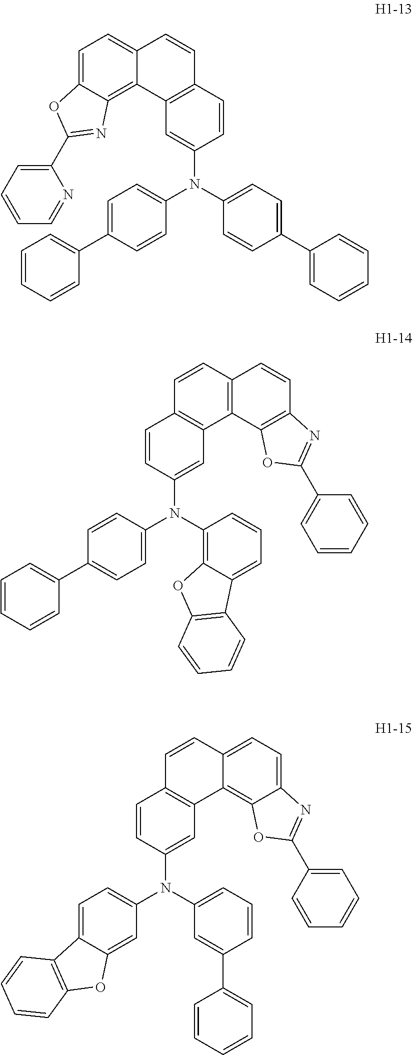 [SMILES: c1ccc(-c2ccc(N(c3ccc(-c4ccccc4)cc3)c3ccc4ccc5ccc6oc(-c7ccccn7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1 ; c1ccc(-c2cccc(N(c3ccc4c(c3)oc3ccccc34)c3ccc4ccc5ccc6nc(-c7ccccc7)oc6c5c4c3)c2)cc1]

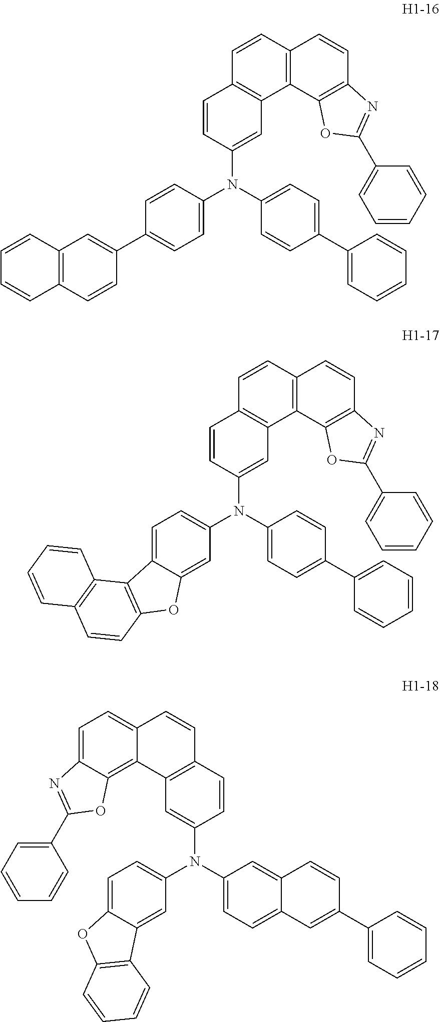 [SMILES: c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1]

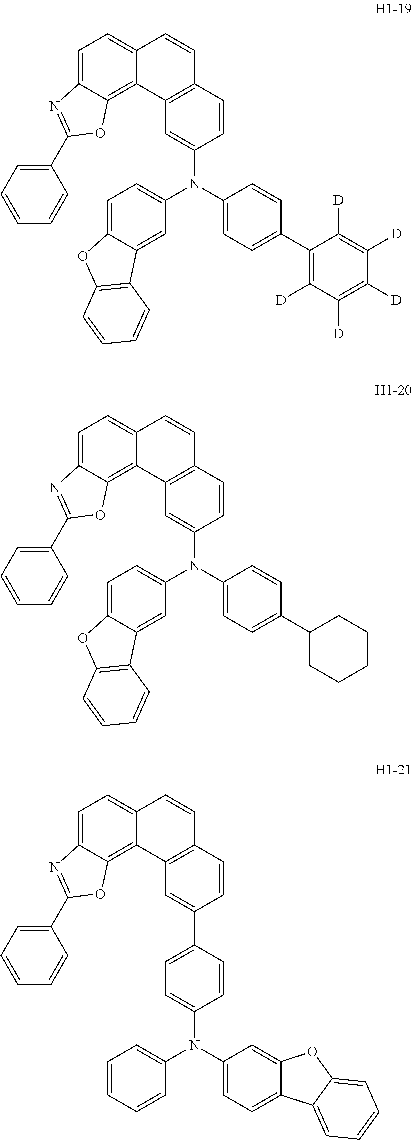 [SMILES: [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2nc3ccc4ccc5ccc(N(c6ccc(C7CCCCC7)cc6)c6ccc7oc8ccccc8c7c6)cc5c4c3o2)cc1 ; c1ccc(-c2nc3ccc4ccc5ccc(-c6ccc(N(c7ccccc7)c7ccc8c(c7)oc7ccccc78)cc6)cc5c4c3o2)cc1]

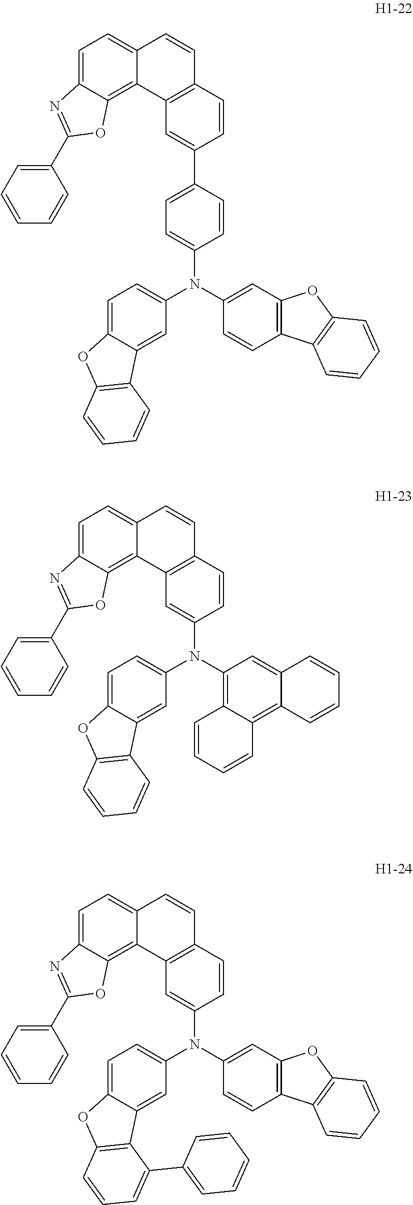 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1]

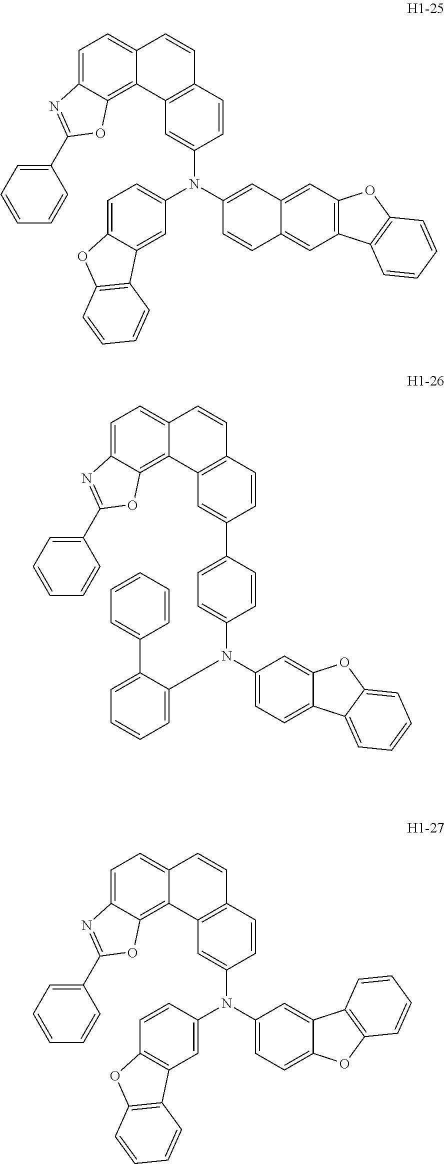 [SMILES: c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1]

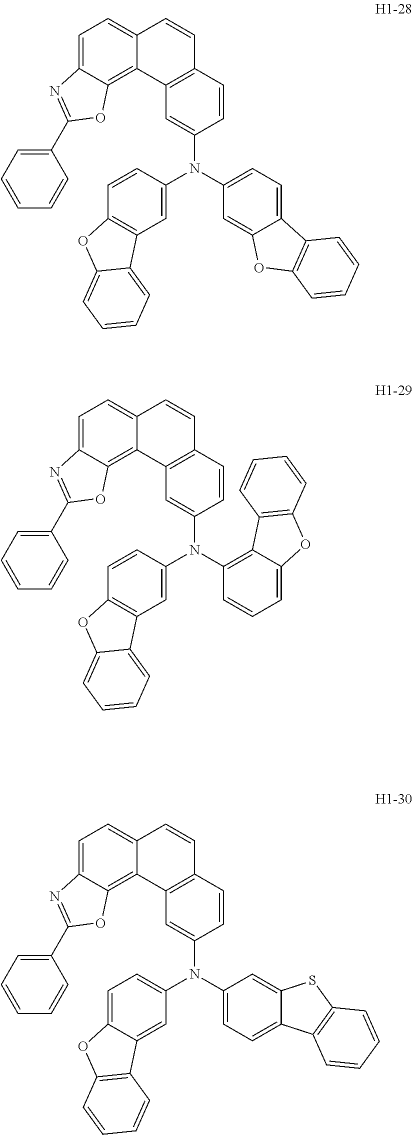 [SMILES: c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1]

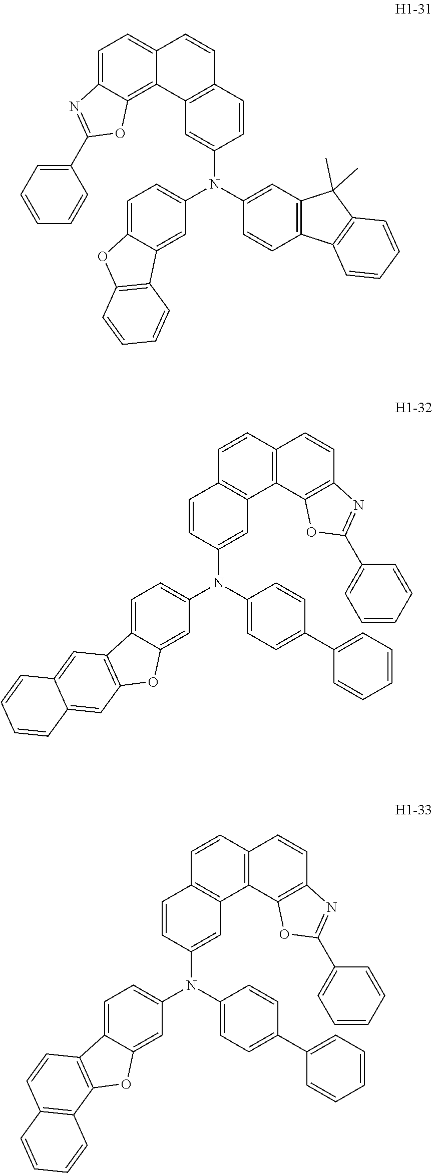 [SMILES: CC1(C)c2ccccc2-c2ccc(N(c3ccc4oc5ccccc5c4c3)c3ccc4ccc5ccc6nc(-c7ccccc7)oc6c5c4c3)cc21 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1]

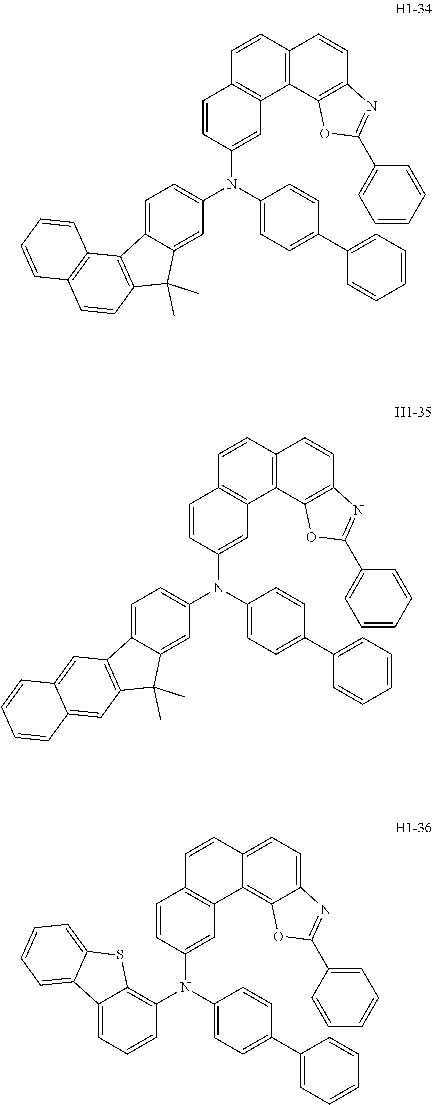 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6nc(-c7ccccc7)oc6c5c4c3)c3cccc4c3sc3ccccc34)cc2)cc1]

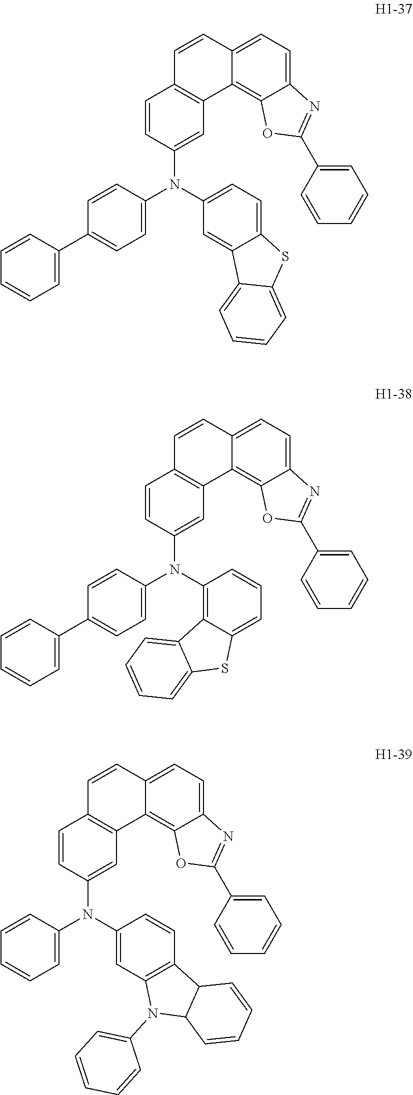 [SMILES: c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1]

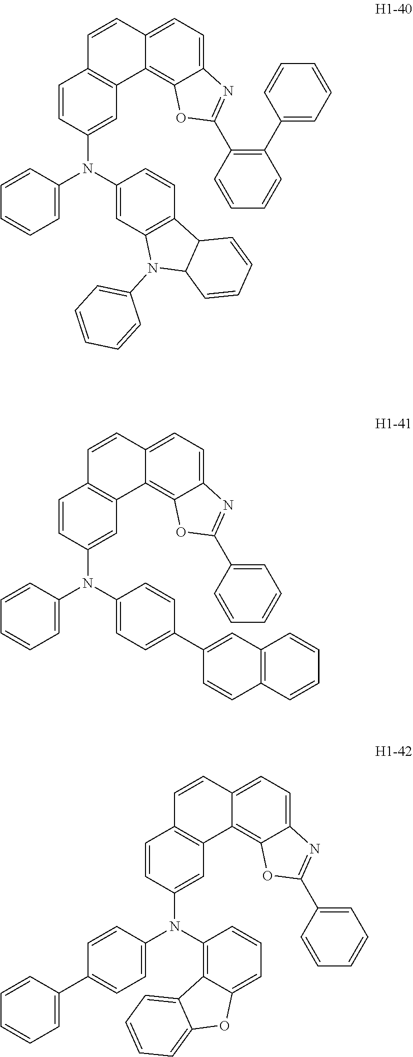 [SMILES: C1=CC2c3ccc(N(c4ccccc4)c4ccc5ccc6ccc7nc(-c8ccccc8-c8ccccc8)oc7c6c5c4)cc3N(c3ccccc3)C2C=C1 ; c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1]

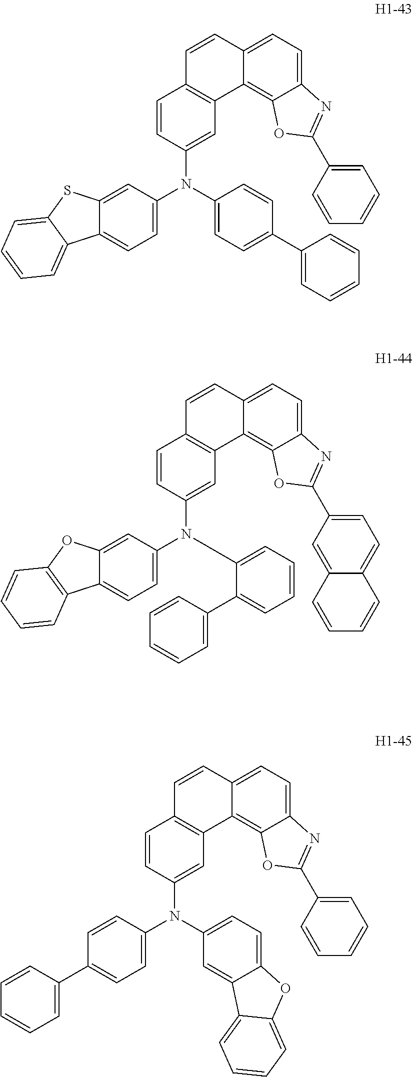 [SMILES: c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccccc2N(c2ccc3c(c2)oc2ccccc23)c2ccc3ccc4ccc5nc(-c6ccc7ccccc7c6)oc5c4c3c2)cc1 ; c1ccc(-c2ccc(N(c3ccc4oc5ccccc5c4c3)c3ccc4ccc5ccc6nc(-c7ccccc7)oc6c5c4c3)cc2)cc1]

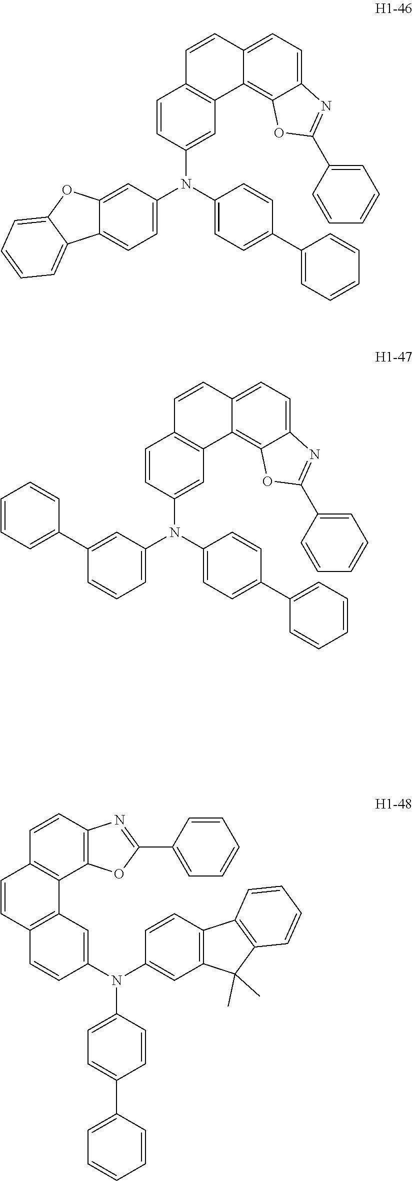 [SMILES: c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21]

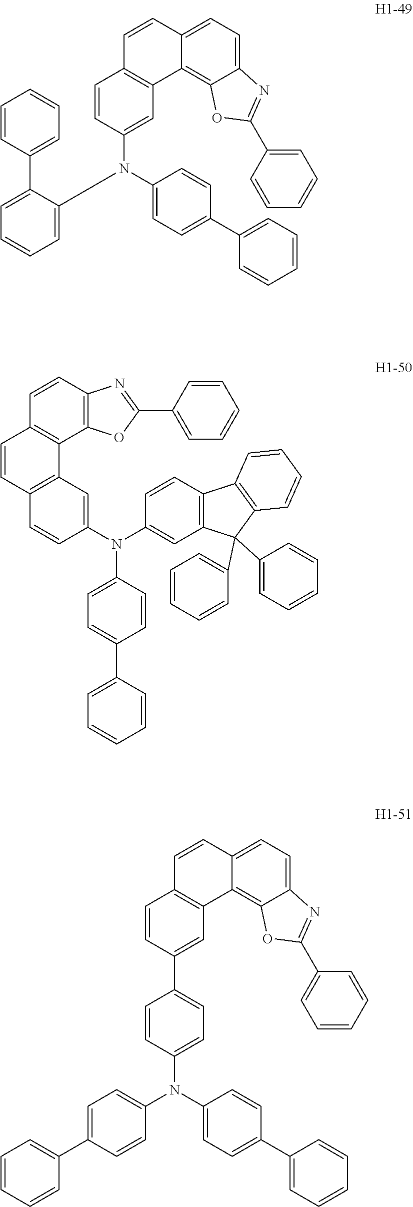 [SMILES: c1ccc(-c2ccc(N(c3ccc4ccc5ccc6nc(-c7ccccc7)oc6c5c4c3)c3ccccc3-c3ccccc3)cc2)cc1 ; c1ccc(-c2ccc(N(c3ccc4c(c3)C(c3ccccc3)(c3ccccc3)c3ccccc3-4)c3ccc4ccc5ccc6nc(-c7ccccc7)oc6c5c4c3)cc2)cc1 ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1]

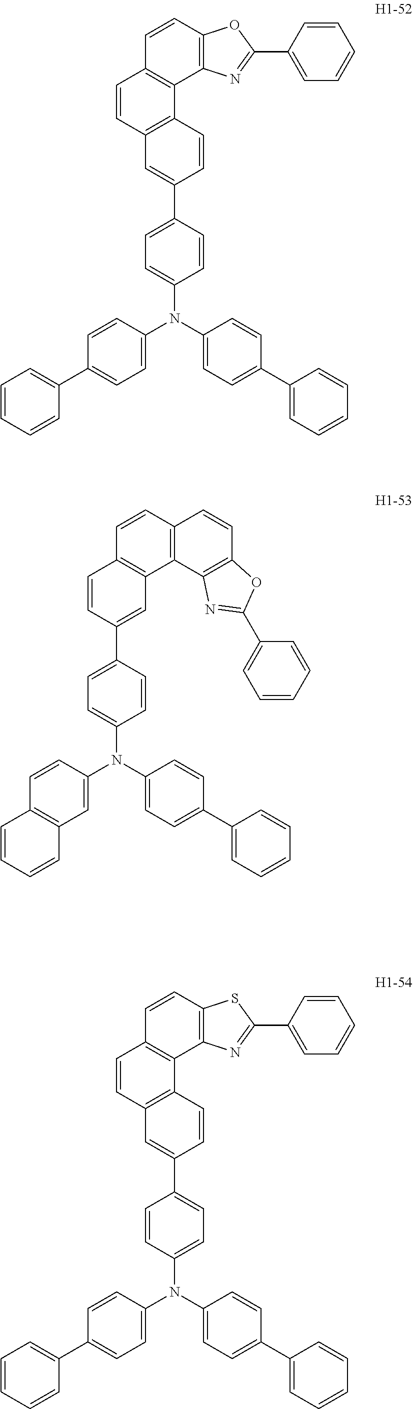 [SMILES: c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1]

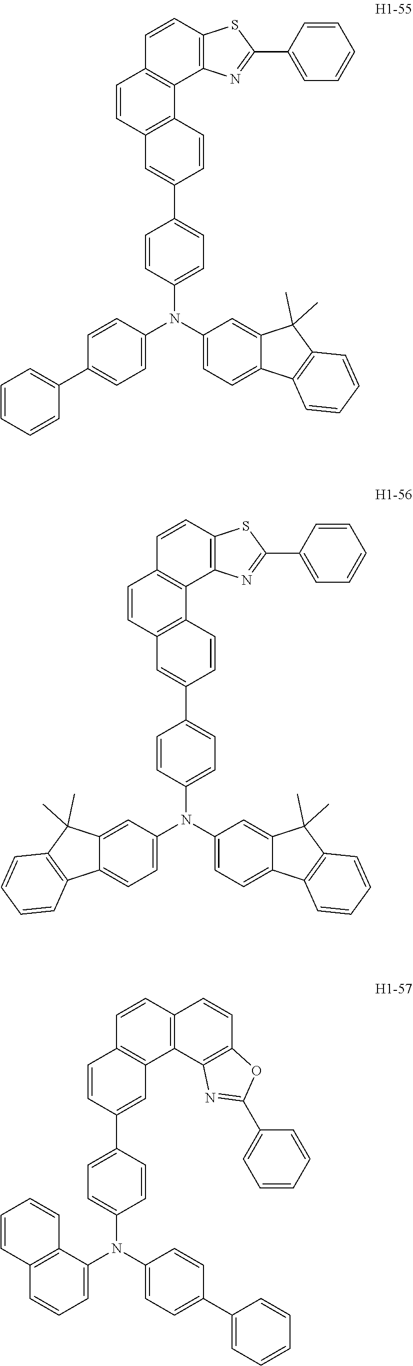 [SMILES: CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(N(c3ccc(-c4ccc5ccc6ccc7oc(-c8ccccc8)nc7c6c5c4)cc3)c3cccc4ccccc34)cc2)cc1]

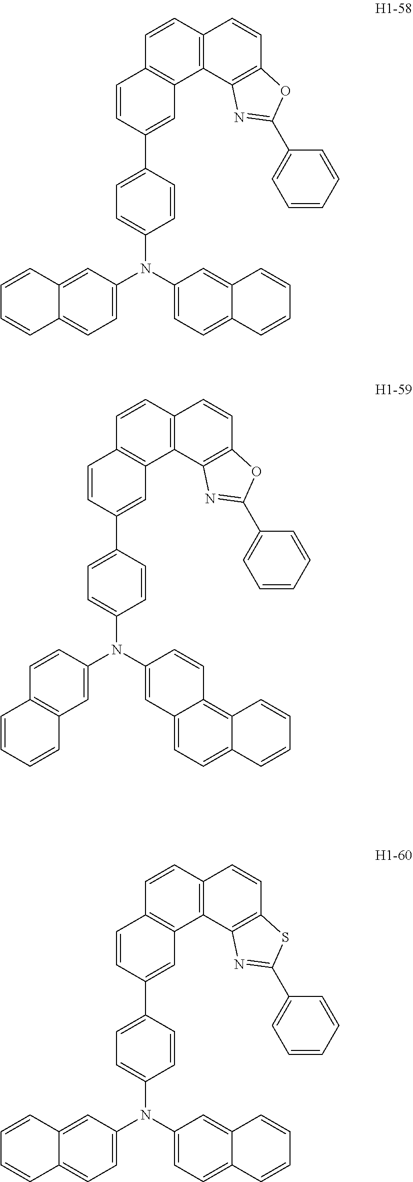 [SMILES: c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1 ; c1ccc(-c2nc3c(ccc4ccc5ccc(-c6ccc(N(c7ccc8ccccc8c7)c7ccc8ccccc8c7)cc6)cc5c43)s2)cc1]

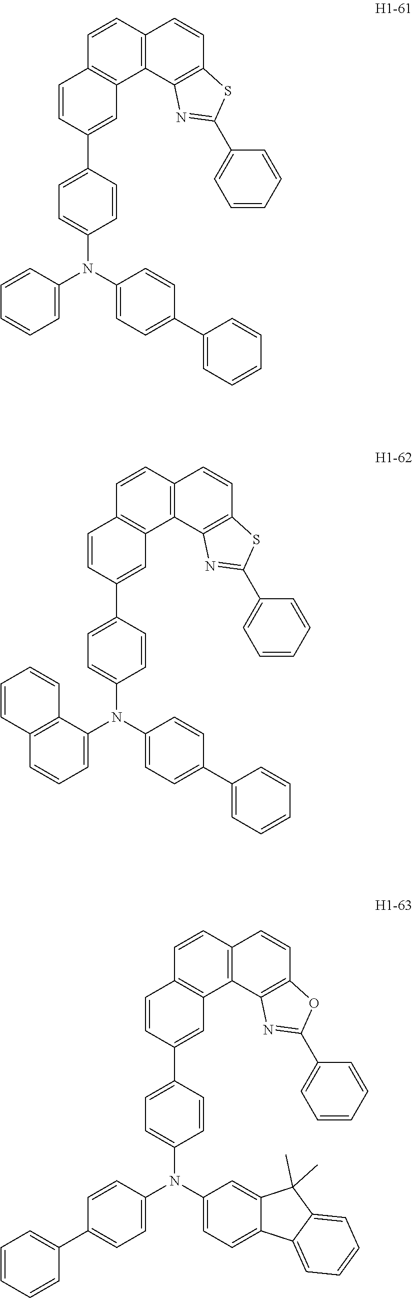 [SMILES: c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21]

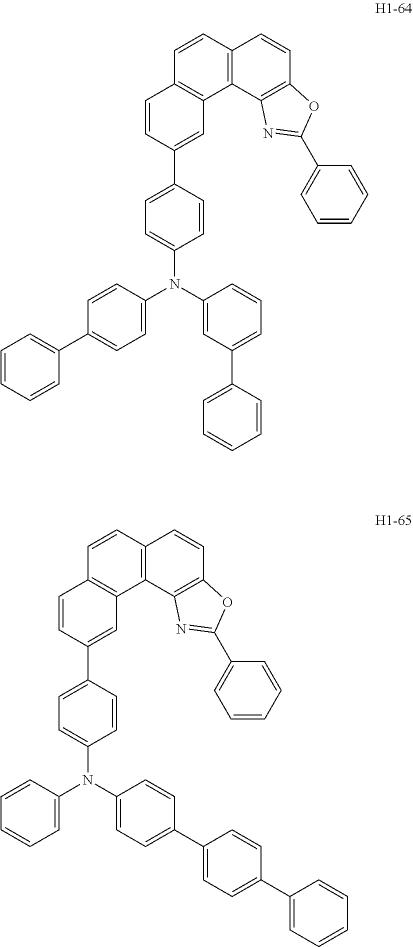 [SMILES: c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

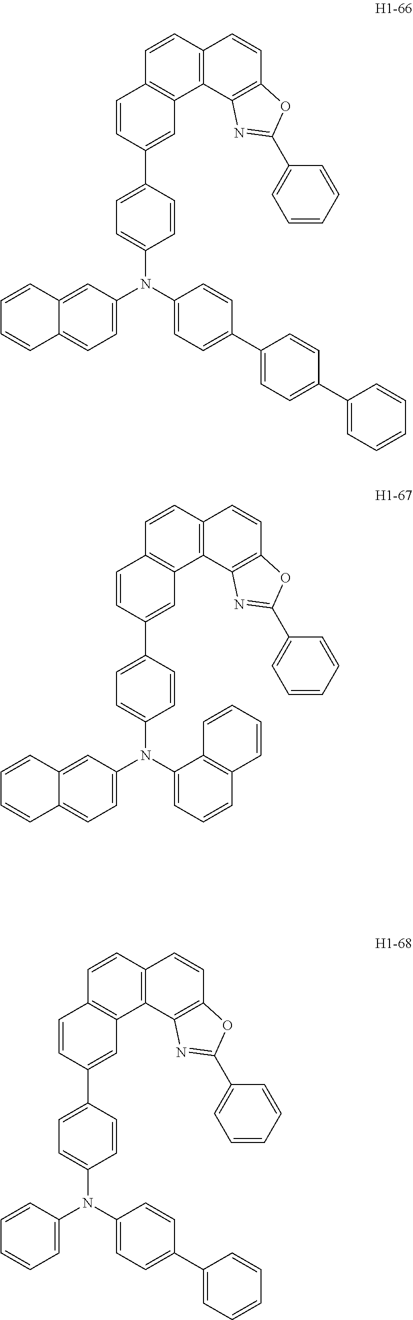 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

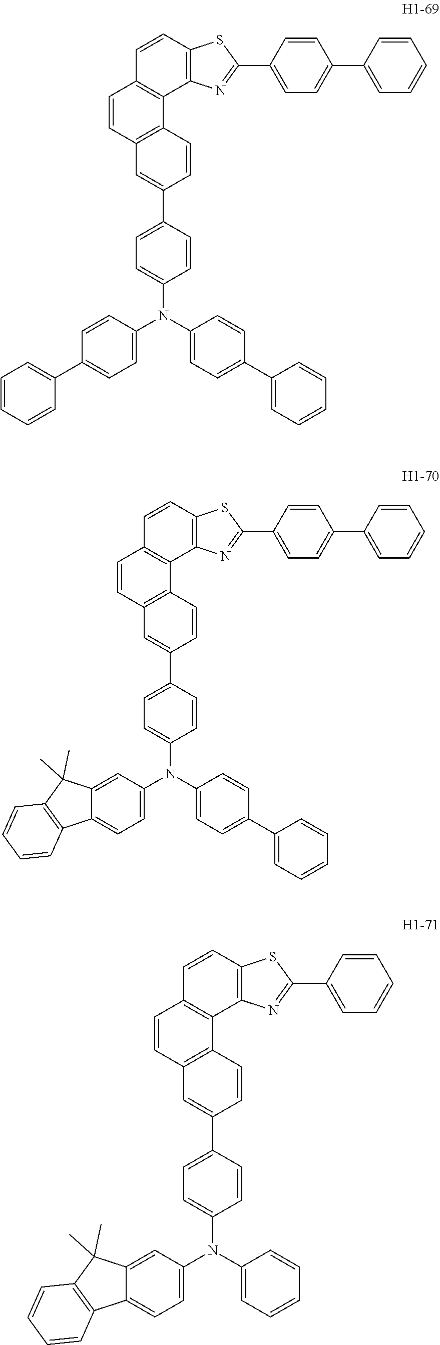 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

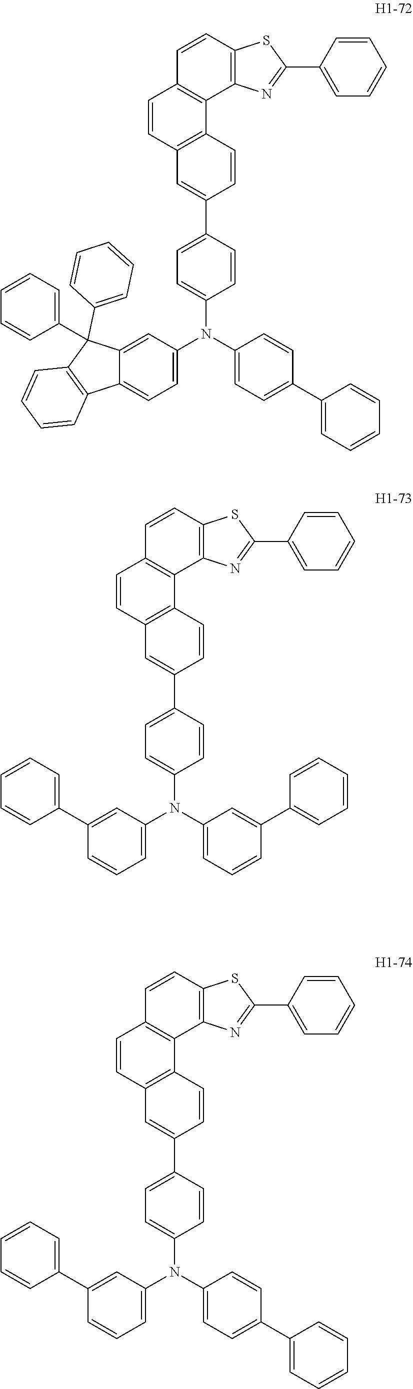 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

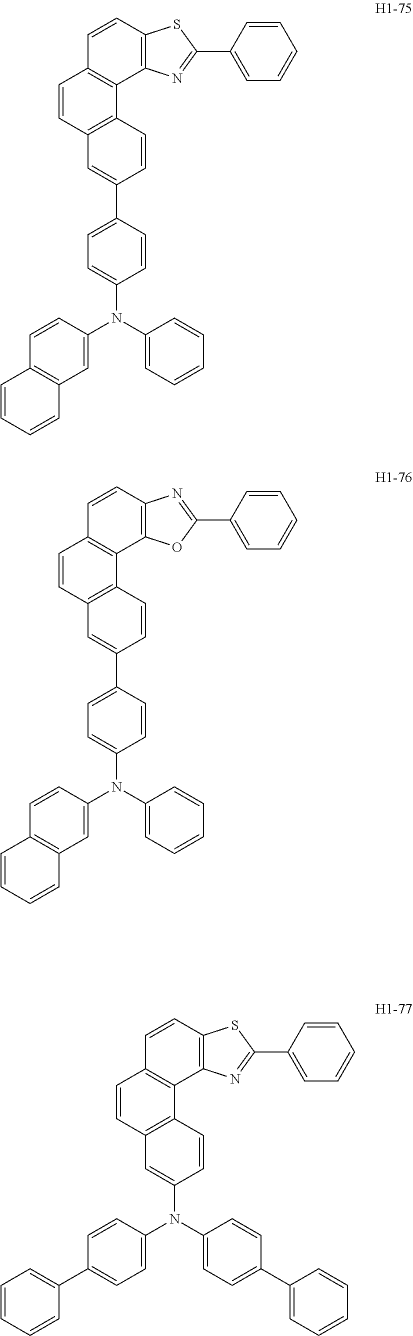 [SMILES: c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1]

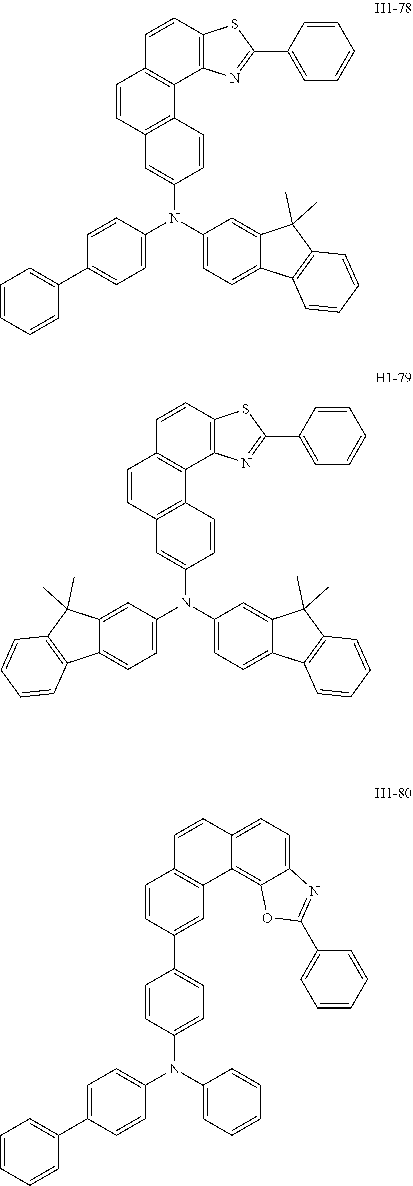 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

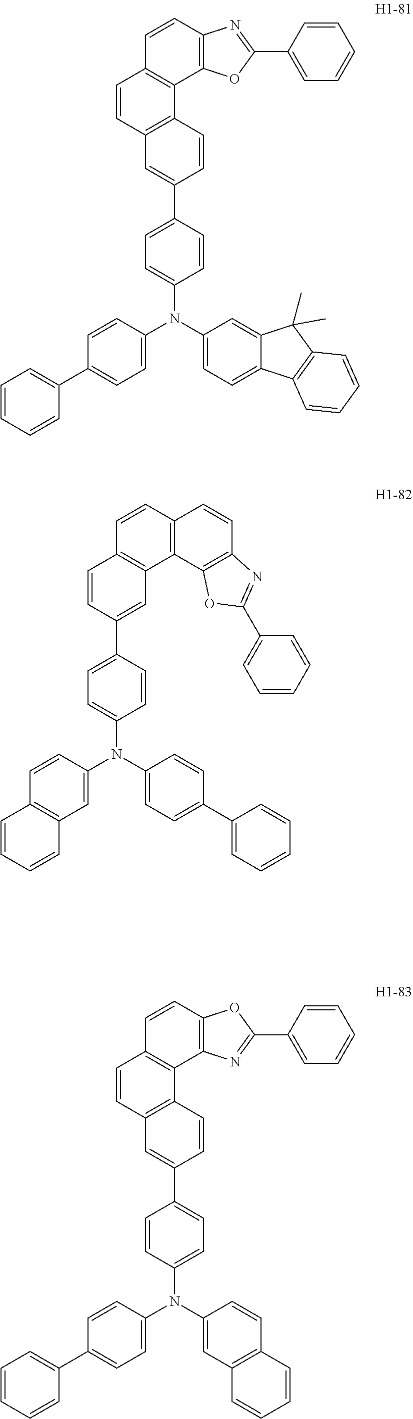 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

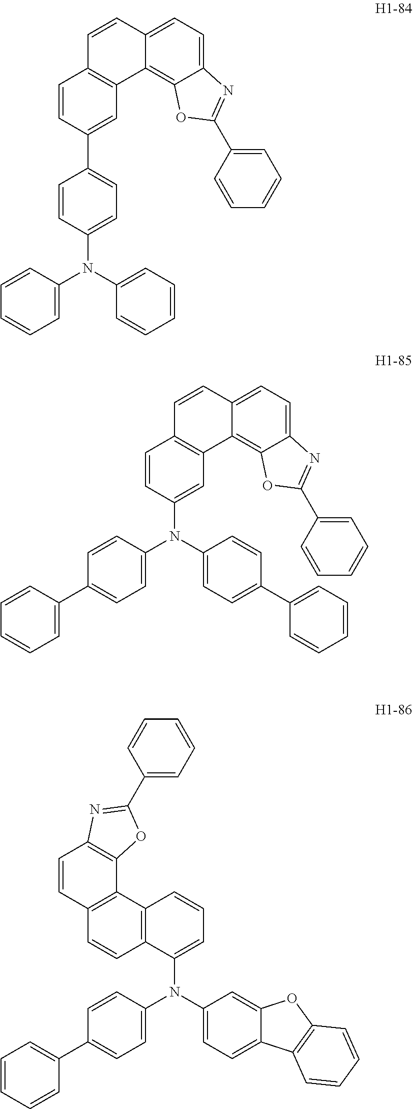 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1]

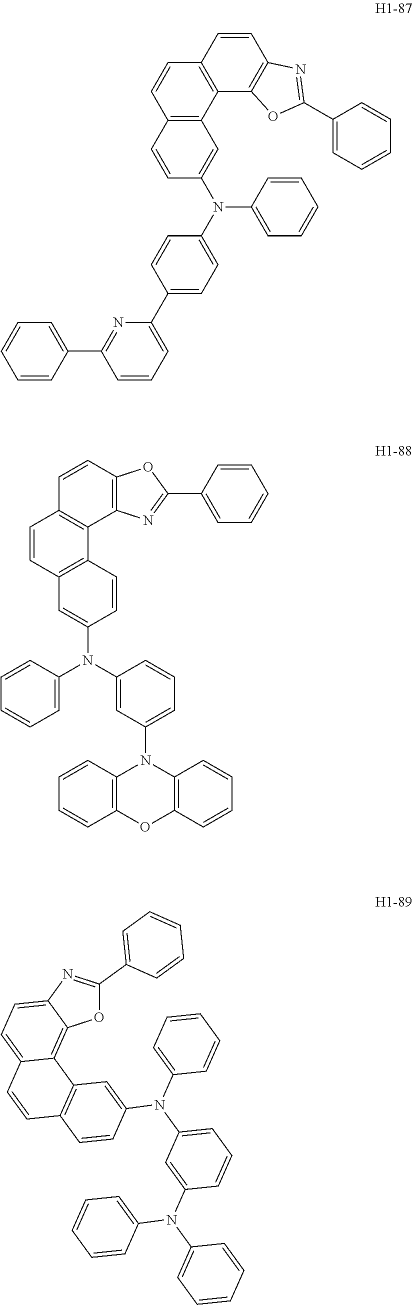 [SMILES: c1ccc(-c2cccc(-c3ccc(N(c4ccccc4)c4ccc5ccc6ccc7nc(-c8ccccc8)oc7c6c5c4)cc3)n2)cc1 ; c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1]

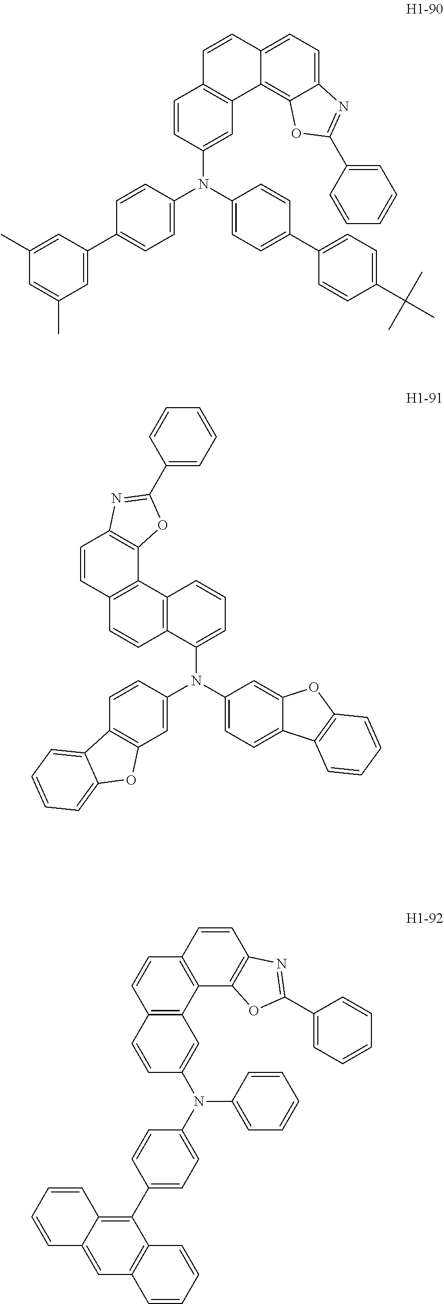 [SMILES: Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2nc3ccc4ccc5ccc(N(c6ccccc6)c6ccc(-c7c8ccccc8cc8ccccc78)cc6)cc5c4c3o2)cc1]

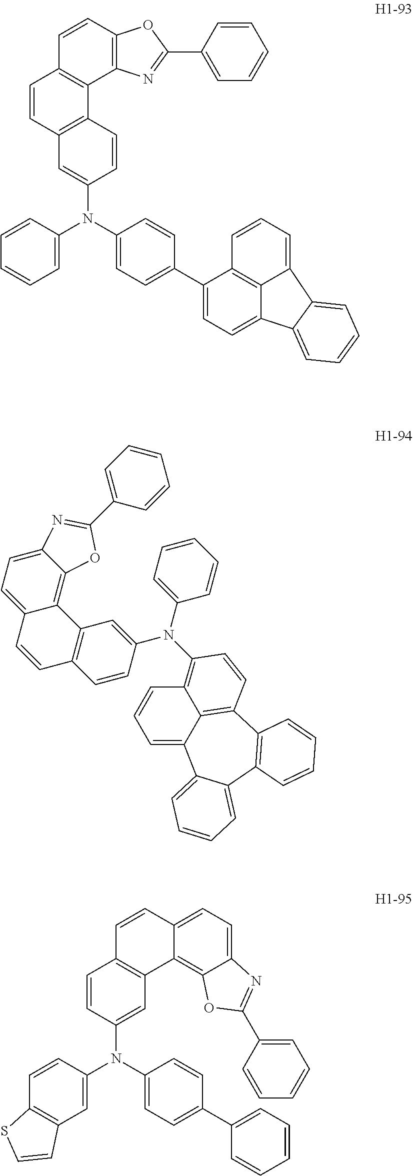 [SMILES: c1ccc(-c2nc3c(ccc4ccc5cc(N(c6ccccc6)c6ccc(-c7ccc8c9c(cccc79)-c7ccccc7-8)cc6)ccc5c43)o2)cc1 ; c1ccc(-c2nc3ccc4ccc5ccc(N(c6ccccc6)c6ccc7c8c(cccc68)-c6ccccc6-c6ccccc6-7)cc5c4c3o2)cc1 ; c1ccc(-c2ccc(N(c3ccc4sccc4c3)c3ccc4ccc5ccc6nc(-c7ccccc7)oc6c5c4c3)cc2)cc1]

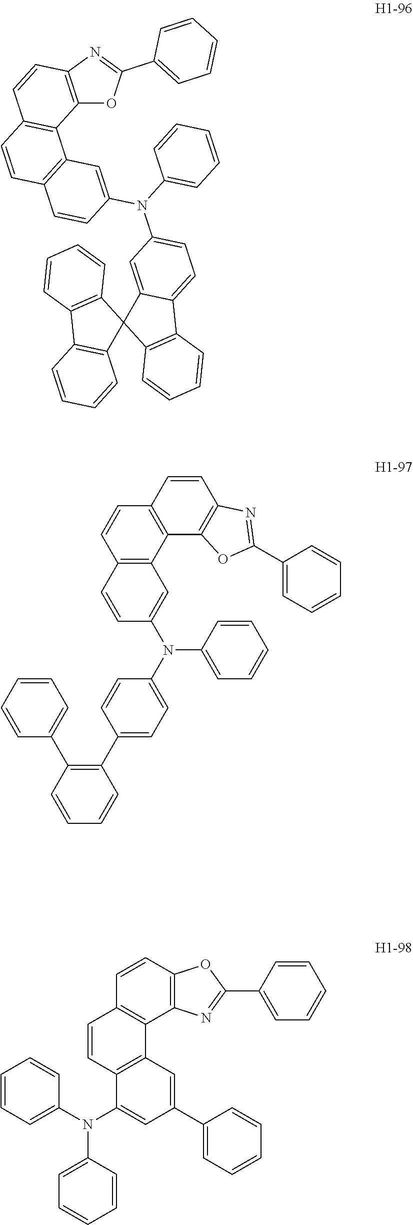 [SMILES: c1ccc(-c2nc3ccc4ccc5ccc(N(c6ccccc6)c6ccc7c(c6)C6(c8ccccc8-c8ccccc86)c6ccccc6-7)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1]

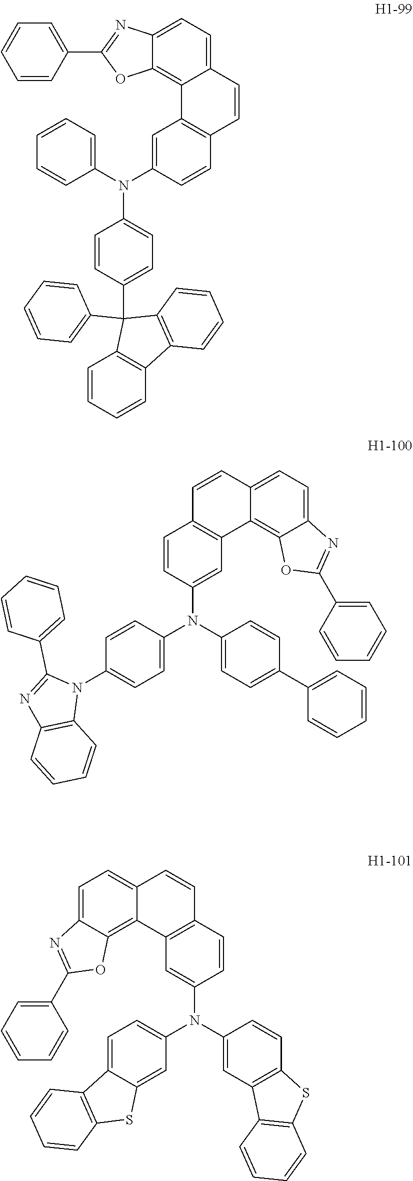 [SMILES: c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1 ; c1ccc(-c2nc3ccc4ccc5ccc(N(c6ccc7c(c6)sc6ccccc67)c6ccc7sc8ccccc8c7c6)cc5c4c3o2)cc1]

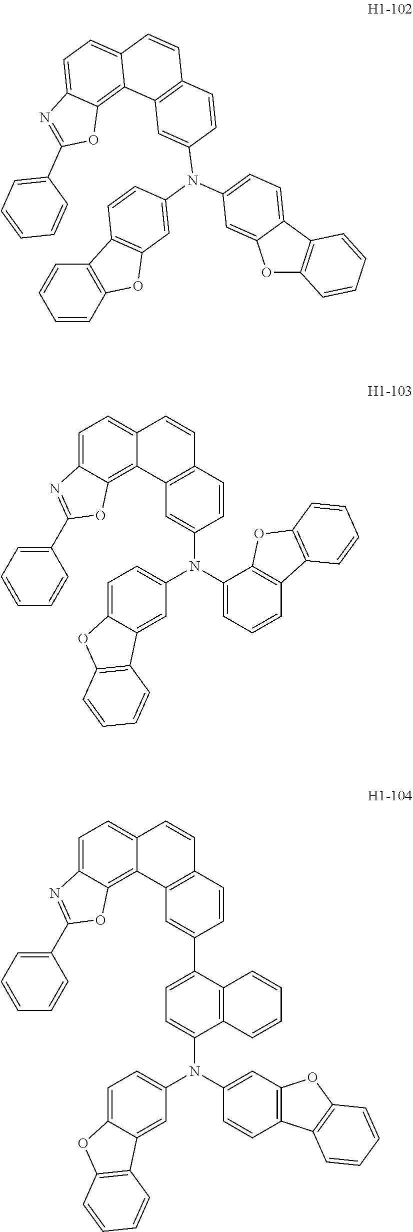 [SMILES: c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1]

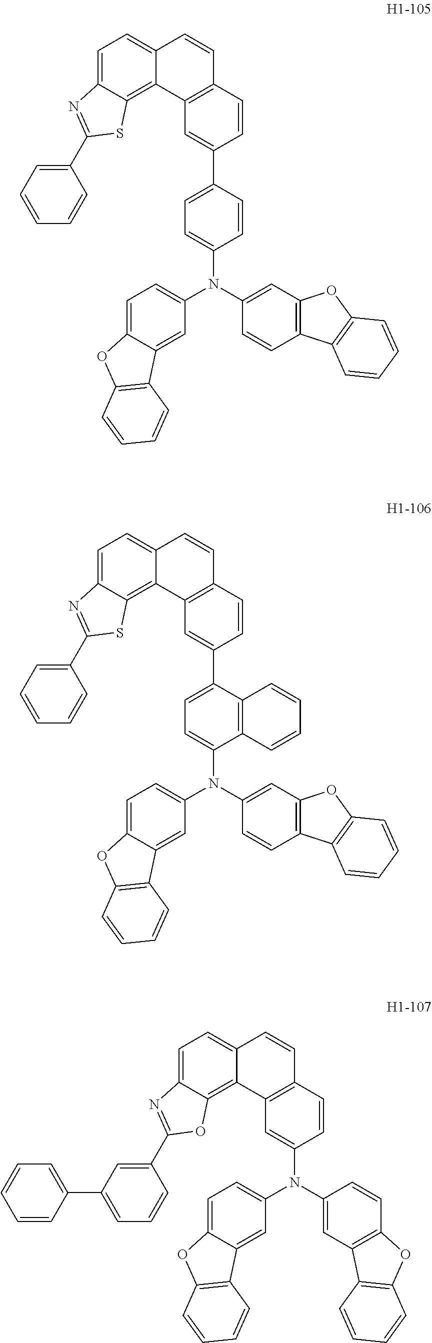 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3s2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3s2)cc1 ; c1ccc(-c2cccc(-c3nc4ccc5ccc6ccc(N(c7ccc8oc9ccccc9c8c7)c7ccc8oc9ccccc9c8c7)cc6c5c4o3)c2)cc1]

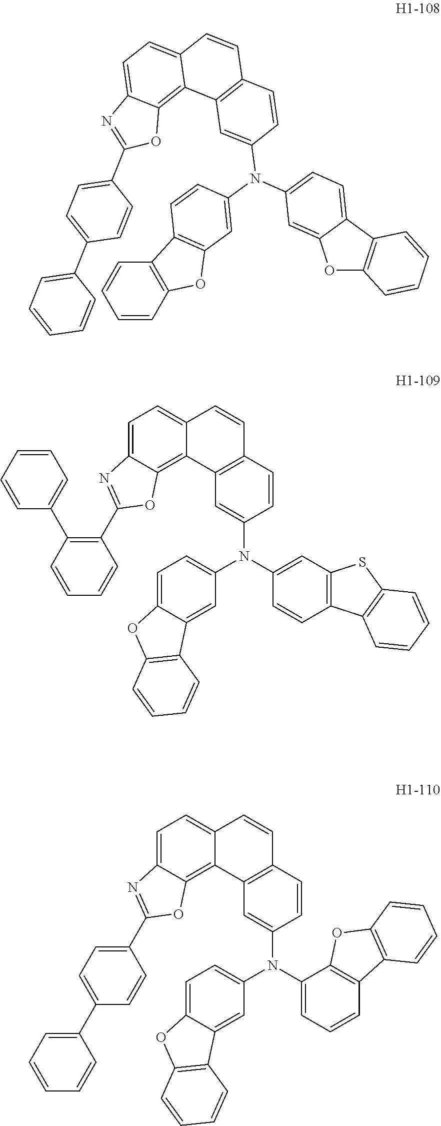 [SMILES: c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1 ; c1ccc(-c2ccc(-c3nc4ccc5ccc6ccc(N(c7ccc8oc9ccccc9c8c7)c7cccc8c7oc7ccccc78)cc6c5c4o3)cc2)cc1]

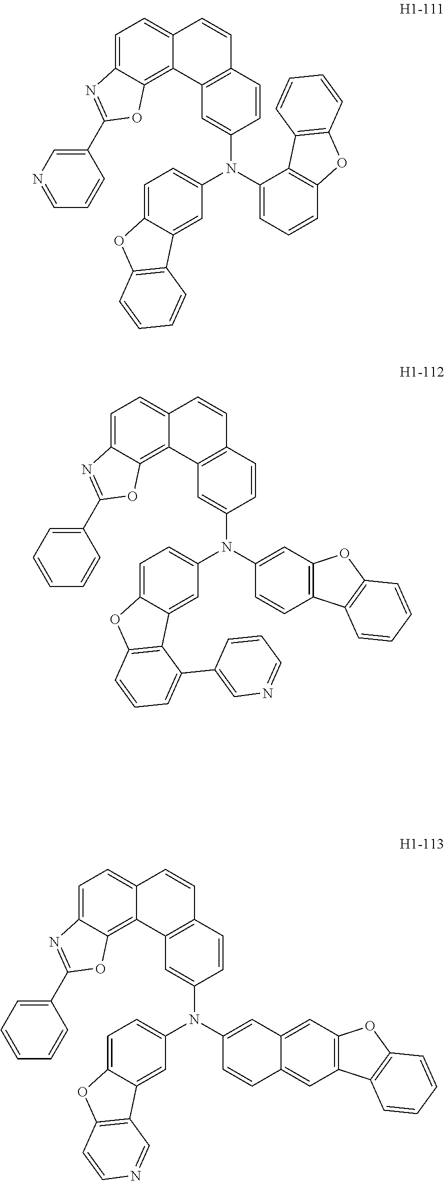 [SMILES: c1cncc(-c2nc3ccc4ccc5ccc(N(c6ccc7oc8ccccc8c7c6)c6cccc7oc8ccccc8c67)cc5c4c3o2)c1 ; c1ccc(-c2nc3ccc4ccc5ccc(N(c6ccc7c(c6)oc6ccccc67)c6ccc7oc8cccc(-c9cccnc9)c8c7c6)cc5c4c3o2)cc1 ; c1ccc(-c2nc3ccc4ccc5ccc(N(c6ccc7cc8c(cc7c6)oc6ccccc68)c6ccc7oc8ccncc8c7c6)cc5c4c3o2)cc1]

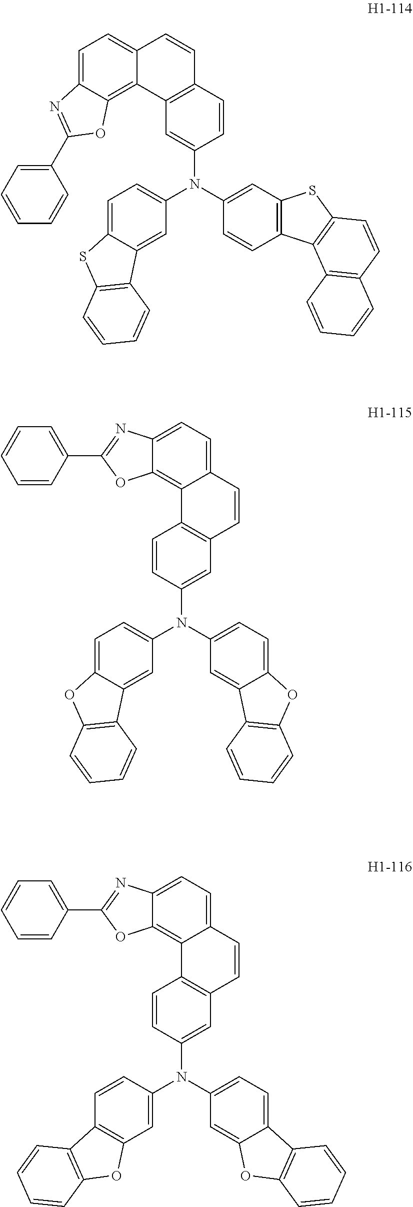 [SMILES: c1ccc(-c2nc3ccc4ccc5ccc(N(c6ccc7c(c6)sc6ccc8ccccc8c67)c6ccc7sc8ccccc8c7c6)cc5c4c3o2)cc1 ; c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1]

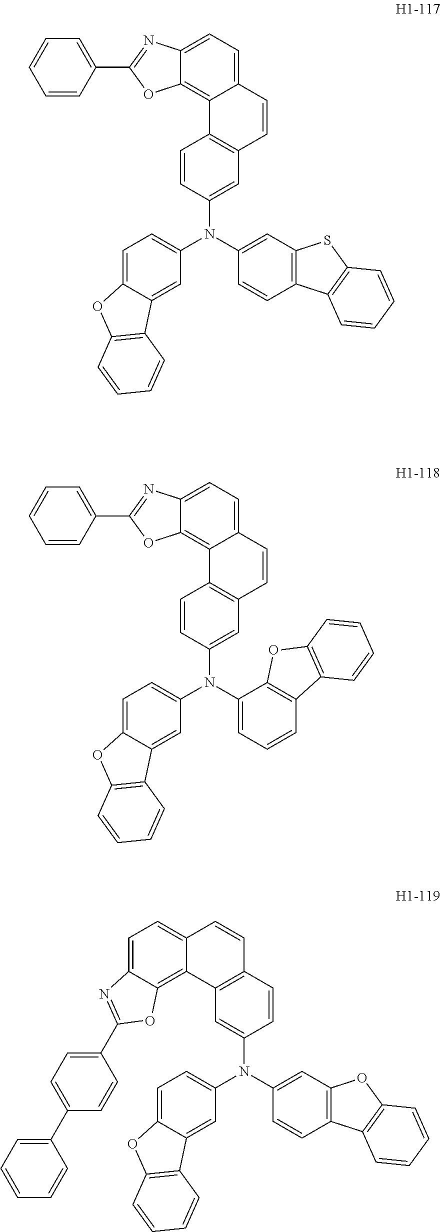 [SMILES: c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7oc8ccccc8c7c6)c6cccc7c6oc6ccccc67)ccc5c4c3o2)cc1 ; c1ccc(-c2ccc(-c3nc4ccc5ccc6ccc(N(c7ccc8c(c7)oc7ccccc78)c7ccc8oc9ccccc9c8c7)cc6c5c4o3)cc2)cc1]

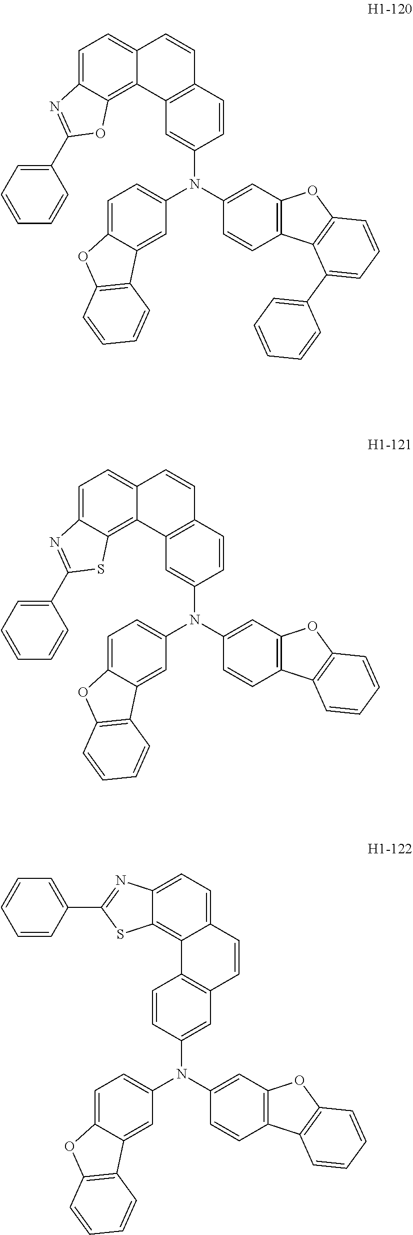 [SMILES: c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2nc3ccc4ccc5cc(N(c6ccc7c(c6)oc6ccccc67)c6ccc7oc8ccccc8c7c6)ccc5c4c3s2)cc1]

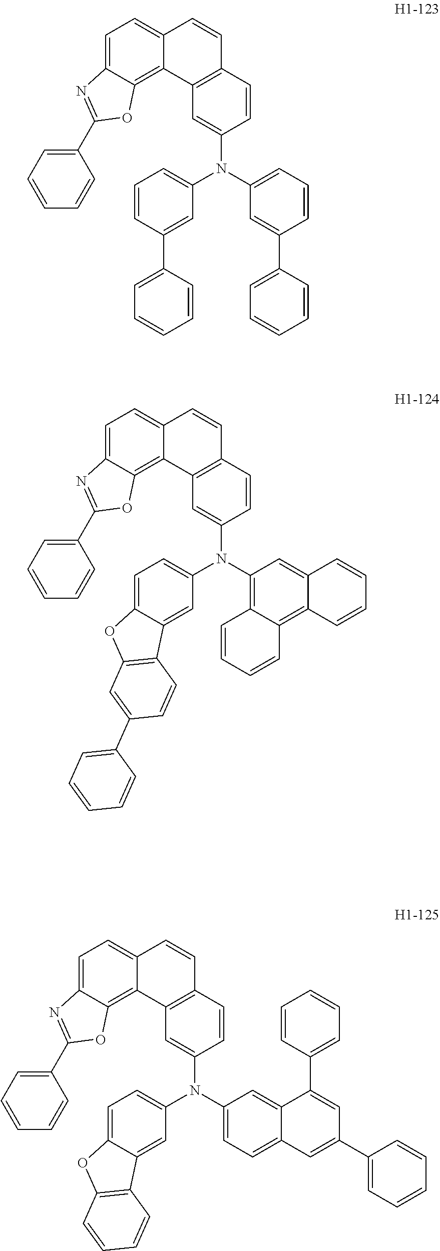 [SMILES: c1ccc(-c2cccc(N(c3cccc(-c4ccccc4)c3)c3ccc4ccc5ccc6nc(-c7ccccc7)oc6c5c4c3)c2)cc1 ; c1ccc(-c2ccc3c(c2)oc2ccc(N(c4ccc5ccc6ccc7nc(-c8ccccc8)oc7c6c5c4)c4cc5ccccc5c5ccccc45)cc23)cc1 ; c1ccc(-c2cc(-c3ccccc3)c3cc(N(c4ccc5oc6ccccc6c5c4)c4ccc5ccc6ccc7nc(-c8ccccc8)oc7c6c5c4)ccc3c2)cc1]

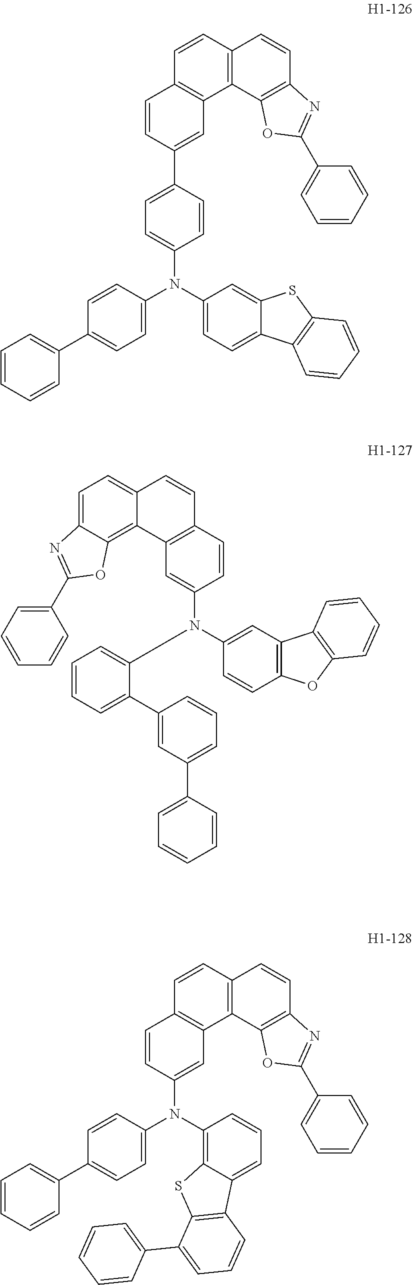 [SMILES: c1ccc(-c2ccc(N(c3ccc(-c4ccc5ccc6ccc7nc(-c8ccccc8)oc7c6c5c4)cc3)c3ccc4c(c3)sc3ccccc34)cc2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3cccc4c3sc3c(-c5ccccc5)cccc34)cc2)cc1]

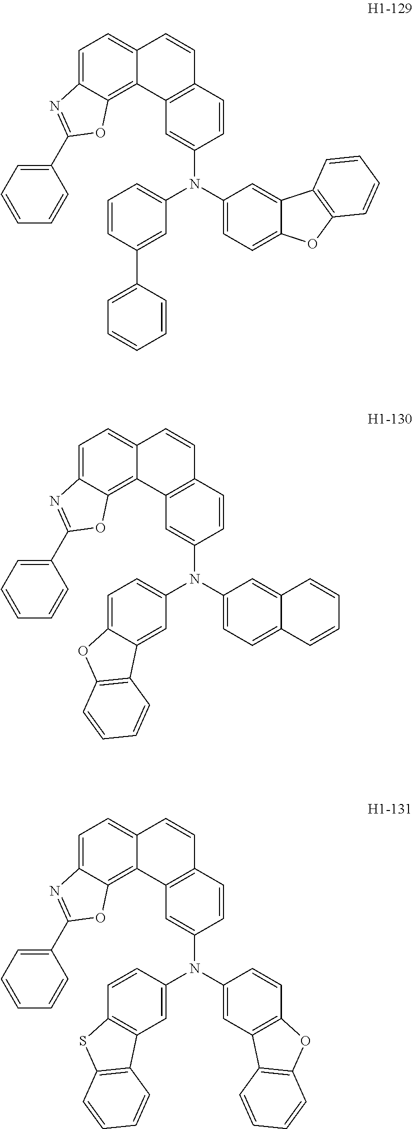 [SMILES: c1ccc(-c2cccc(N(c3ccc4oc5ccccc5c4c3)c3ccc4ccc5ccc6nc(-c7ccccc7)oc6c5c4c3)c2)cc1 ; c1ccc(-c2nc3ccc4ccc5ccc(N(c6ccc7ccccc7c6)c6ccc7oc8ccccc8c7c6)cc5c4c3o2)cc1 ; c1ccc(-c2nc3ccc4ccc5ccc(N(c6ccc7oc8ccccc8c7c6)c6ccc7sc8ccccc8c7c6)cc5c4c3o2)cc1]

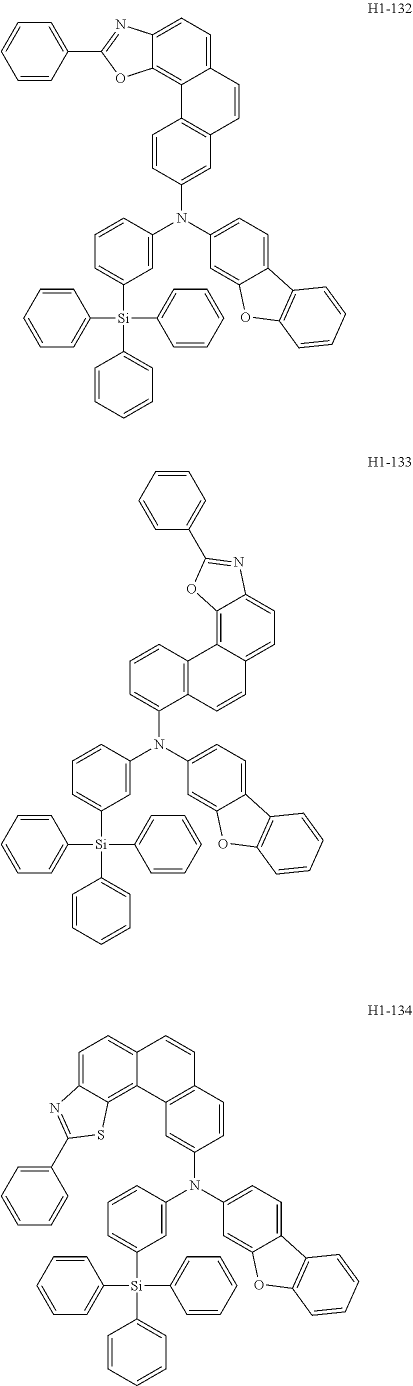 [SMILES: c1ccc(-c2nc3ccc4ccc5cc(N(c6cccc([Si](c7ccccc7)(c7ccccc7)c7ccccc7)c6)c6ccc7c(c6)oc6ccccc67)ccc5c4c3o2)cc1 ; c1ccc(-c2nc3ccc4ccc5c(N(c6cccc([Si](c7ccccc7)(c7ccccc7)c7ccccc7)c6)c6ccc7c(c6)oc6ccccc67)cccc5c4c3o2)cc1 ; c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3s2)cc1]

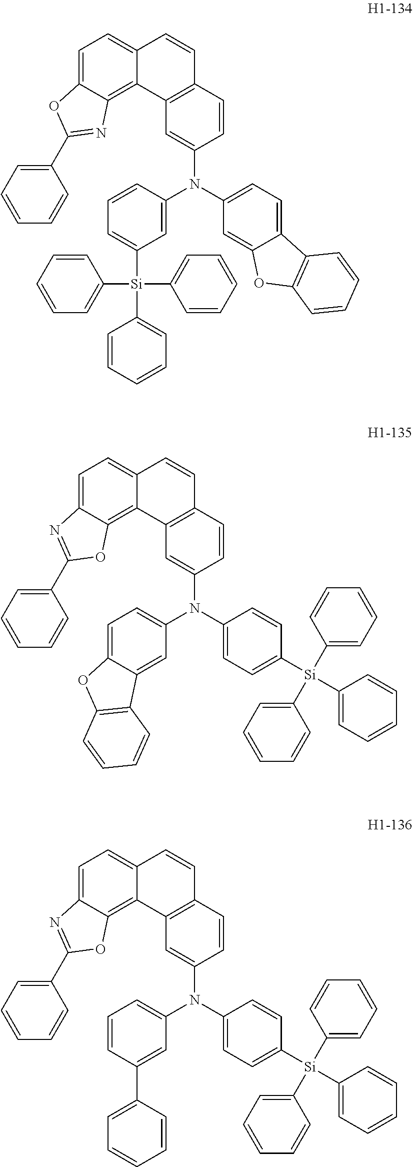 [SMILES: c1ccc(-c2nc3c(ccc4ccc5ccc(N(c6cccc([Si](c7ccccc7)(c7ccccc7)c7ccccc7)c6)c6ccc7c(c6)oc6ccccc67)cc5c43)o2)cc1 ; c1ccc(-c2nc3ccc4ccc5ccc(N(c6ccc([Si](c7ccccc7)(c7ccccc7)c7ccccc7)cc6)c6ccc7oc8ccccc8c7c6)cc5c4c3o2)cc1 ; c1ccc(-c2cccc(N(c3ccc([Si](c4ccccc4)(c4ccccc4)c4ccccc4)cc3)c3ccc4ccc5ccc6nc(-c7ccccc7)oc6c5c4c3)c2)cc1]

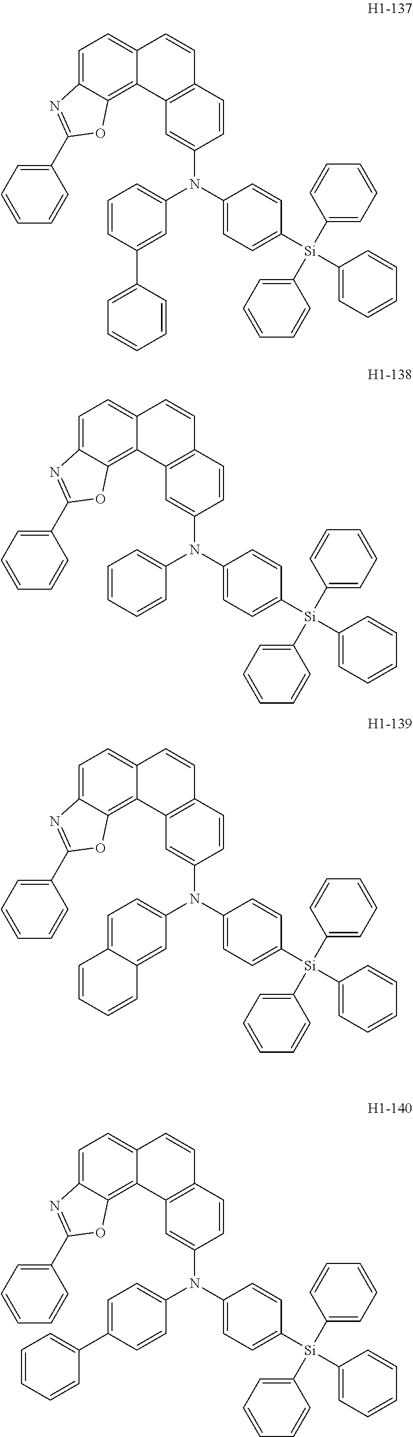 [SMILES: c1ccc(-c2cccc(N(c3ccc([Si](c4ccccc4)(c4ccccc4)c4ccccc4)cc3)c3ccc4ccc5ccc6nc(-c7ccccc7)oc6c5c4c3)c2)cc1 ; c1ccc(-c2nc3ccc4ccc5ccc(N(c6ccccc6)c6ccc([Si](c7ccccc7)(c7ccccc7)c7ccccc7)cc6)cc5c4c3o2)cc1 ; c1ccc(-c2nc3ccc4ccc5ccc(N(c6ccc([Si](c7ccccc7)(c7ccccc7)c7ccccc7)cc6)c6ccc7ccccc7c6)cc5c4c3o2)cc1 ; c1ccc(-c2ccc(N(c3ccc([Si](c4ccccc4)(c4ccccc4)c4ccccc4)cc3)c3ccc4ccc5ccc6nc(-c7ccccc7)oc6c5c4c3)cc2)cc1]

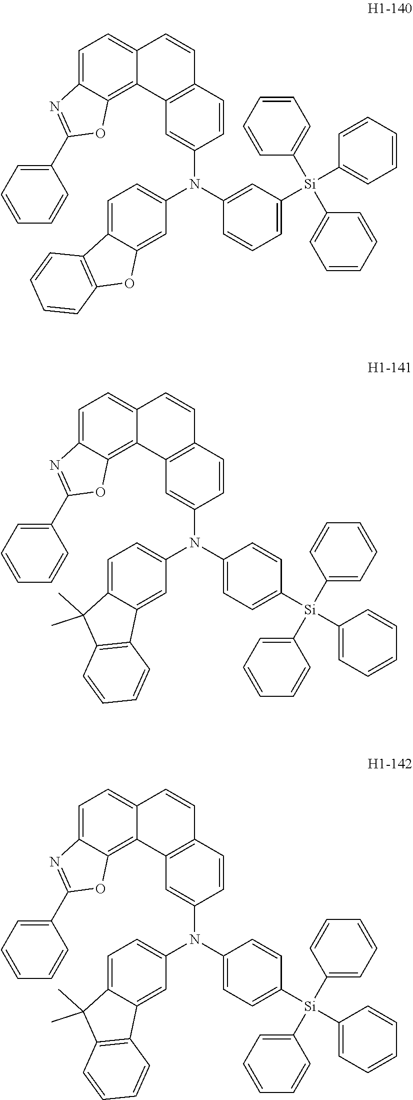 [SMILES: c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3o2)cc1 ; CC1(C)c2ccccc2-c2cc(N(c3ccc([Si](c4ccccc4)(c4ccccc4)c4ccccc4)cc3)c3ccc4ccc5ccc6nc(-c7ccccc7)oc6c5c4c3)ccc21 ; CC1(C)c2ccccc2-c2cc(N(c3ccc([Si](c4ccccc4)(c4ccccc4)c4ccccc4)cc3)c3ccc4ccc5ccc6nc(-c7ccccc7)oc6c5c4c3)ccc21]

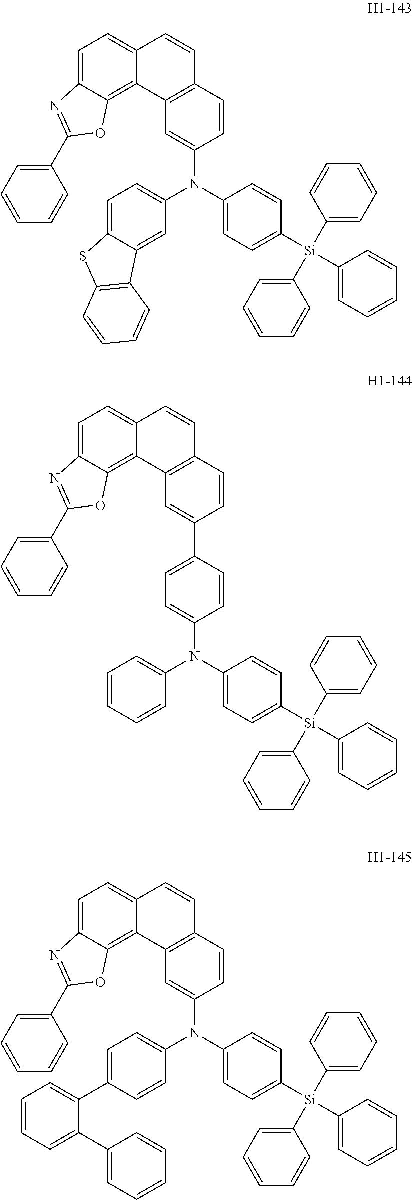 [SMILES: c1ccc(-c2nc3ccc4ccc5ccc(N(c6ccc([Si](c7ccccc7)(c7ccccc7)c7ccccc7)cc6)c6ccc7sc8ccccc8c7c6)cc5c4c3o2)cc1 ; c1ccc(-c2nc3ccc4ccc5ccc(-c6ccc(N(c7ccccc7)c7ccc([Si](c8ccccc8)(c8ccccc8)c8ccccc8)cc7)cc6)cc5c4c3o2)cc1 ; c1ccc(-c2nc3ccc4ccc5ccc(N(c6ccc(-c7ccccc7-c7ccccc7)cc6)c6ccc([Si](c7ccccc7)(c7ccccc7)c7ccccc7)cc6)cc5c4c3o2)cc1]

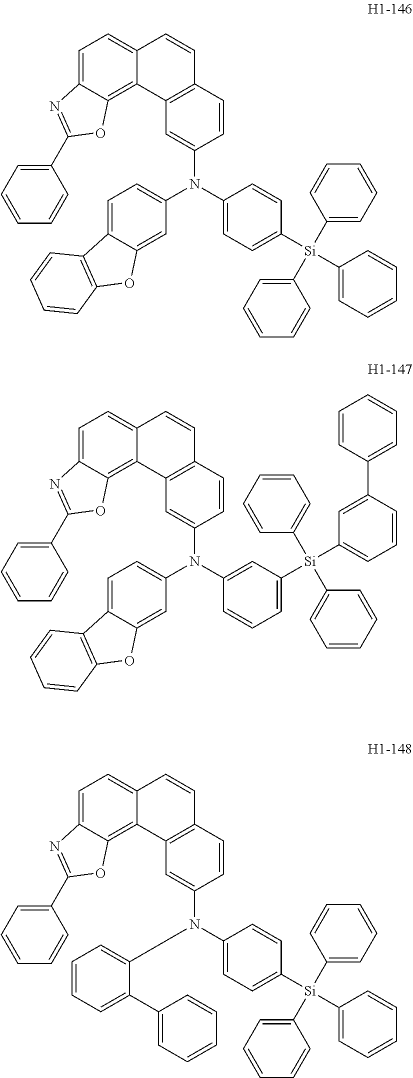 [SMILES: c1ccc(-c2nc3ccc4ccc5ccc(N(c6ccc([Si](c7ccccc7)(c7ccccc7)c7ccccc7)cc6)c6ccc7c(c6)oc6ccccc67)cc5c4c3o2)cc1 ; c1ccc(-c2cccc([Si](c3ccccc3)(c3ccccc3)c3cccc(N(c4ccc5c(c4)oc4ccccc45)c4ccc5ccc6ccc7nc(-c8ccccc8)oc7c6c5c4)c3)c2)cc1 ; c1ccc(-c2nc3ccc4ccc5ccc(N(c6ccc([Si](c7ccccc7)(c7ccccc7)c7ccccc7)cc6)c6ccccc6-c6ccccc6)cc5c4c3o2)cc1]

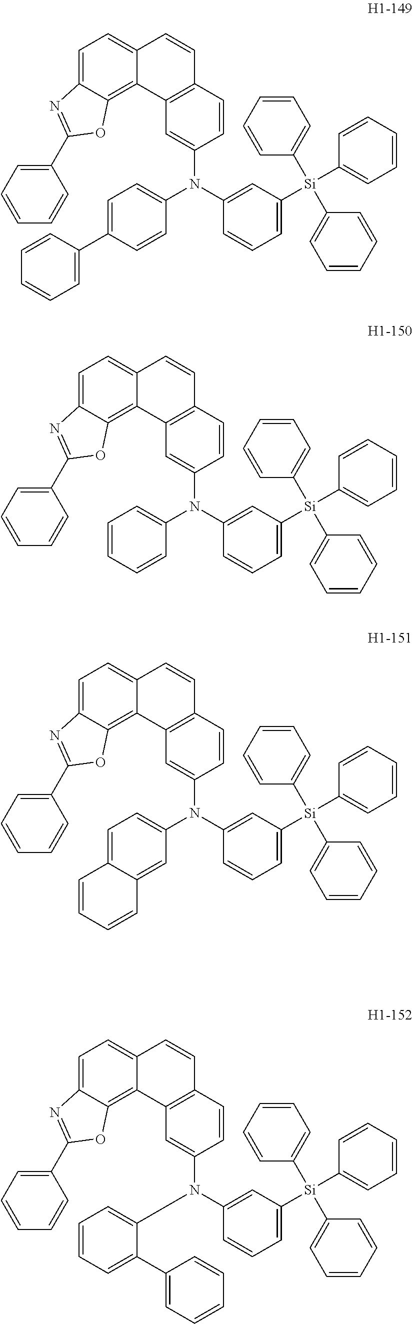 [SMILES: c1ccc(-c2ccc(N(c3cccc([Si](c4ccccc4)(c4ccccc4)c4ccccc4)c3)c3ccc4ccc5ccc6nc(-c7ccccc7)oc6c5c4c3)cc2)cc1 ; c1ccc(-c2nc3ccc4ccc5ccc(N(c6ccccc6)c6cccc([Si](c7ccccc7)(c7ccccc7)c7ccccc7)c6)cc5c4c3o2)cc1 ; c1ccc(-c2nc3ccc4ccc5ccc(N(c6cccc([Si](c7ccccc7)(c7ccccc7)c7ccccc7)c6)c6ccc7ccccc7c6)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6ccccc6)cc5c4c3o2)cc1]

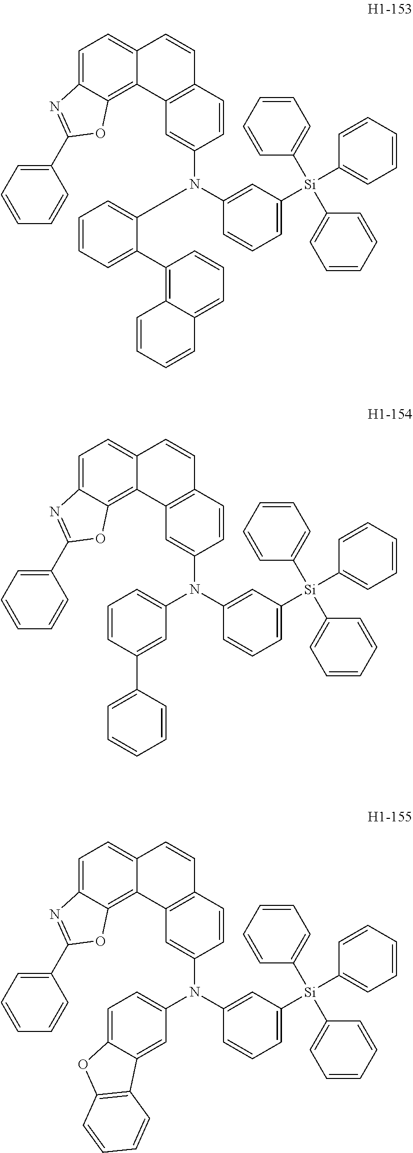 [SMILES: c1ccc(-c2nc3ccc4ccc5ccc(N(c6cccc([Si](c7ccccc7)(c7ccccc7)c7ccccc7)c6)c6ccccc6-c6cccc7ccccc67)cc5c4c3o2)cc1 ; c1ccc(-c2cccc(N(c3cccc([Si](c4ccccc4)(c4ccccc4)c4ccccc4)c3)c3ccc4ccc5ccc6nc(-c7ccccc7)oc6c5c4c3)c2)cc1 ; c1ccc(-c2nc3ccc4ccc5ccc(N(c6cccc([Si](c7ccccc7)(c7ccccc7)c7ccccc7)c6)c6ccc7oc8ccccc8c7c6)cc5c4c3o2)cc1]

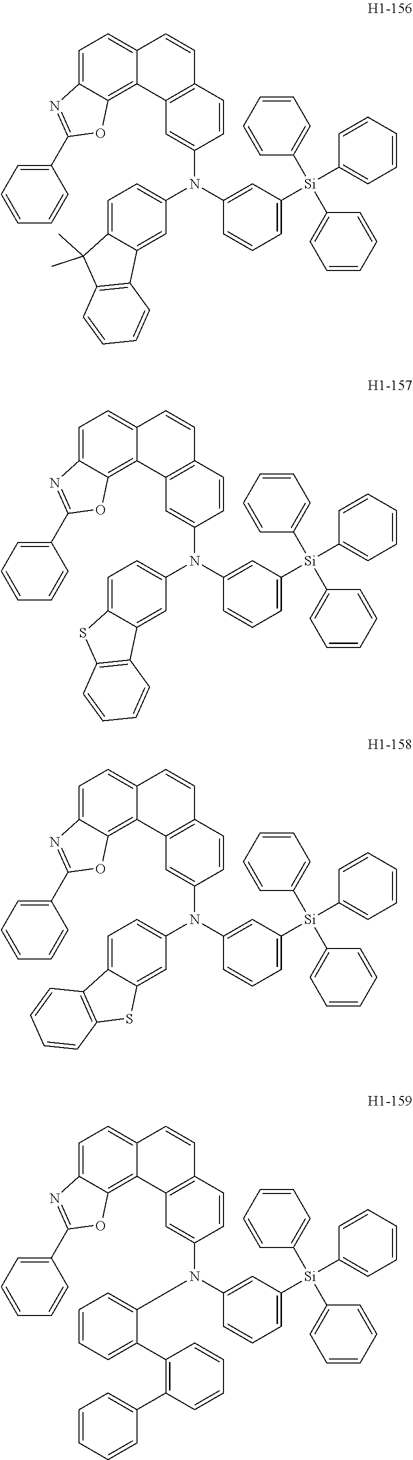 [SMILES: CC1(C)c2ccccc2-c2cc(N(c3cccc([Si](c4ccccc4)(c4ccccc4)c4ccccc4)c3)c3ccc4ccc5ccc6nc(-c7ccccc7)oc6c5c4c3)ccc21 ; c1ccc(-c2nc3ccc4ccc5ccc(N(c6cccc([Si](c7ccccc7)(c7ccccc7)c7ccccc7)c6)c6ccc7sc8ccccc8c7c6)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)sc6ccccc67)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6ccccc6-c6ccccc6)cc5c4c3o2)cc1]

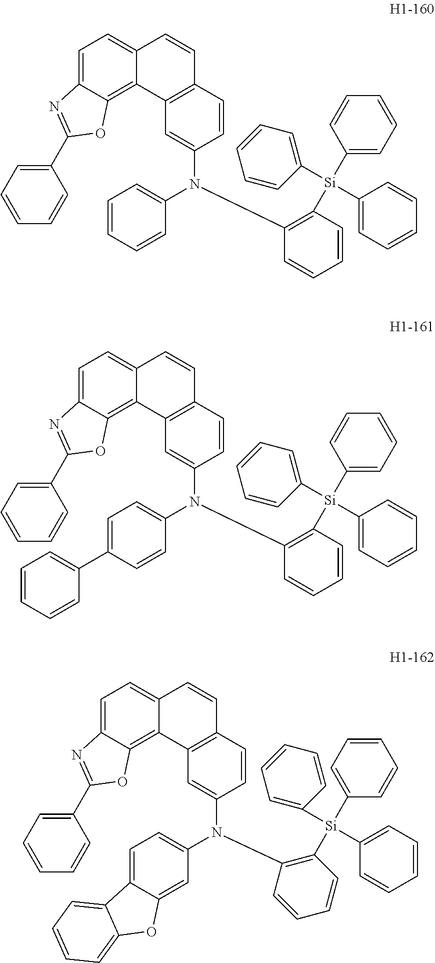 [SMILES: c1ccc(-c2nc3ccc4ccc5ccc(N(c6ccccc6)c6ccccc6[Si](c6ccccc6)(c6ccccc6)c6ccccc6)cc5c4c3o2)cc1 ; c1ccc(-c2ccc(N(c3ccc4ccc5ccc6nc(-c7ccccc7)oc6c5c4c3)c3ccccc3[Si](c3ccccc3)(c3ccccc3)c3ccccc3)cc2)cc1 ; c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1]

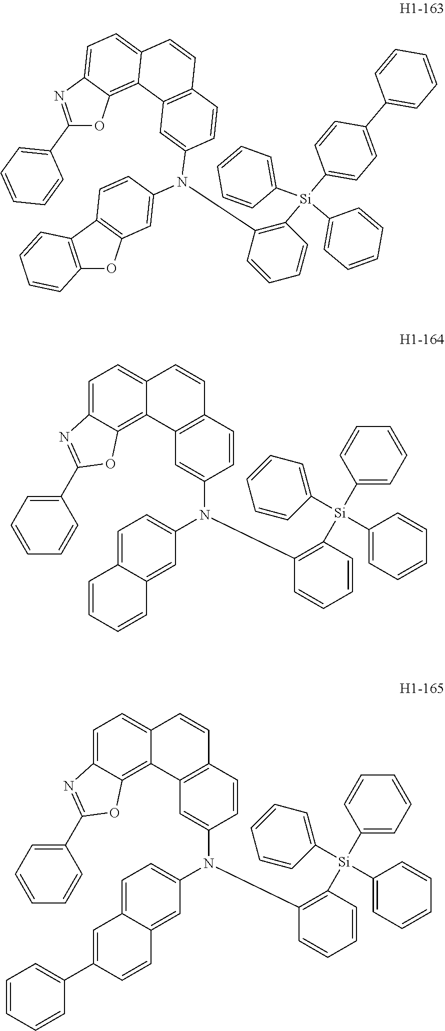 [SMILES: c1ccc(-c2ccc([Si](c3ccccc3)(c3ccccc3)c3ccccc3N(c3ccc4c(c3)oc3ccccc34)c3ccc4ccc5ccc6nc(-c7ccccc7)oc6c5c4c3)cc2)cc1 ; c1ccc(-c2nc3ccc4ccc5ccc(N(c6ccc7ccccc7c6)c6ccccc6[Si](c6ccccc6)(c6ccccc6)c6ccccc6)cc5c4c3o2)cc1 ; c1ccc(-c2ccc3cc(N(c4ccc5ccc6ccc7nc(-c8ccccc8)oc7c6c5c4)c4ccccc4[Si](c4ccccc4)(c4ccccc4)c4ccccc4)ccc3c2)cc1]

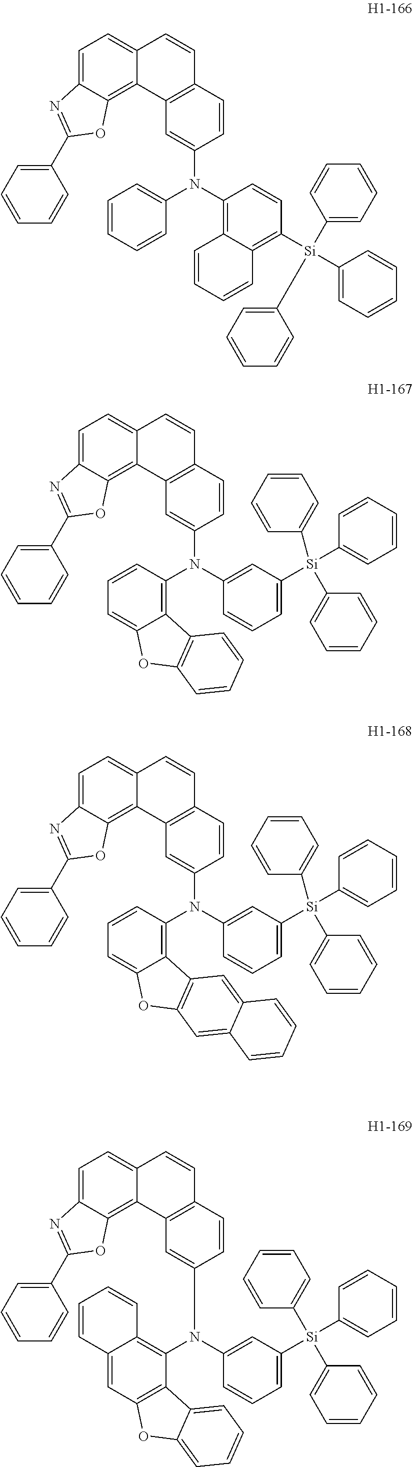 [SMILES: c1ccc(-c2nc3ccc4ccc5ccc(N(c6ccccc6)c6ccc([Si](c7ccccc7)(c7ccccc7)c7ccccc7)c7ccccc67)cc5c4c3o2)cc1 ; c1ccc(-c2nc3ccc4ccc5ccc(N(c6cccc([Si](c7ccccc7)(c7ccccc7)c7ccccc7)c6)c6cccc7oc8ccccc8c67)cc5c4c3o2)cc1 ; c1ccc(-c2nc3ccc4ccc5ccc(N(c6cccc([Si](c7ccccc7)(c7ccccc7)c7ccccc7)c6)c6cccc7oc8cc9ccccc9cc8c67)cc5c4c3o2)cc1 ; c1ccc(-c2nc3ccc4ccc5ccc(N(c6cccc([Si](c7ccccc7)(c7ccccc7)c7ccccc7)c6)c6c7ccccc7cc7oc8ccccc8c67)cc5c4c3o2)cc1]

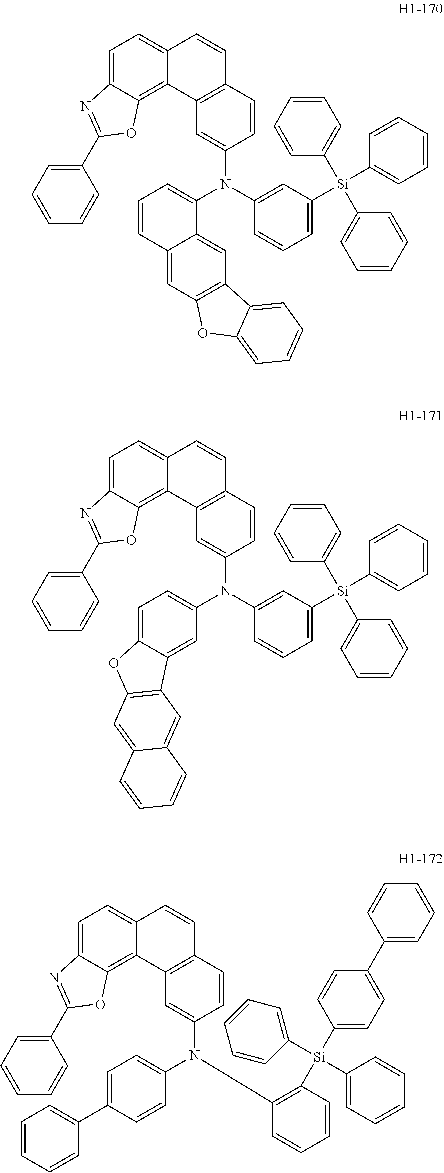 [SMILES: c1ccc(-c2nc3ccc4ccc5ccc(N(c6cccc([Si](c7ccccc7)(c7ccccc7)c7ccccc7)c6)c6cccc7cc8oc9ccccc9c8cc67)cc5c4c3o2)cc1 ; c1ccc(-c2nc3ccc4ccc5ccc(N(c6cccc([Si](c7ccccc7)(c7ccccc7)c7ccccc7)c6)c6ccc7oc8cc9ccccc9cc8c7c6)cc5c4c3o2)cc1 ; c1ccc(-c2ccc(N(c3ccc4ccc5ccc6nc(-c7ccccc7)oc6c5c4c3)c3ccccc3[Si](c3ccccc3)(c3ccccc3)c3ccc(-c4ccccc4)cc3)cc2)cc1]

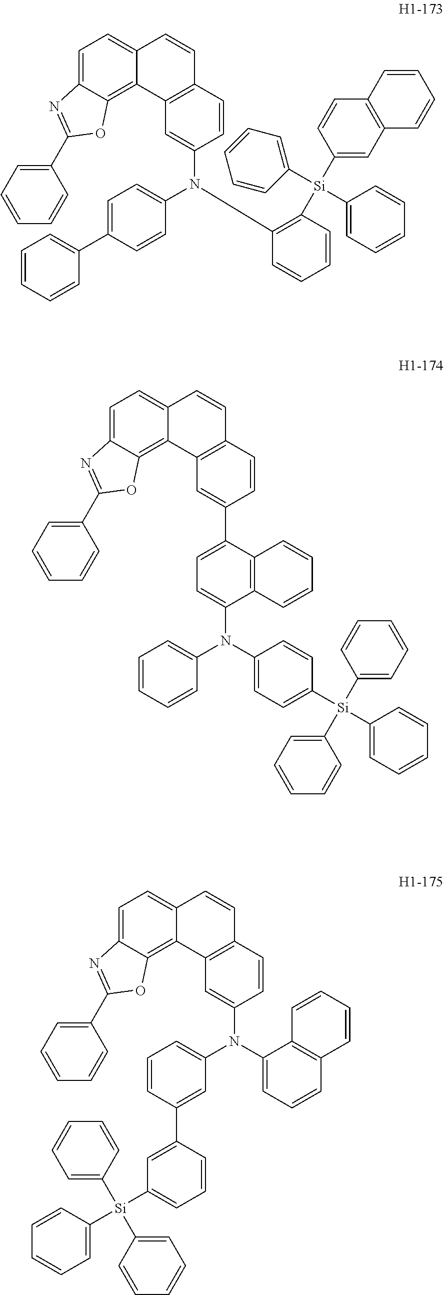 [SMILES: c1ccc(-c2ccc(N(c3ccc4ccc5ccc6nc(-c7ccccc7)oc6c5c4c3)c3ccccc3[Si](c3ccccc3)(c3ccccc3)c3ccc4ccccc4c3)cc2)cc1 ; c1ccc(-c2nc3ccc4ccc5ccc(-c6ccc(N(c7ccccc7)c7ccc([Si](c8ccccc8)(c8ccccc8)c8ccccc8)cc7)c7ccccc67)cc5c4c3o2)cc1 ; c1ccc(-c2nc3ccc4ccc5ccc(N(c6cccc(-c7cccc([Si](c8ccccc8)(c8ccccc8)c8ccccc8)c7)c6)c6cccc7ccccc67)cc5c4c3o2)cc1]

According to one embodiment, the organic electroluminescent compound represented by formula 3 may be more specifically illustrated by the following compounds, but is not limited thereto.

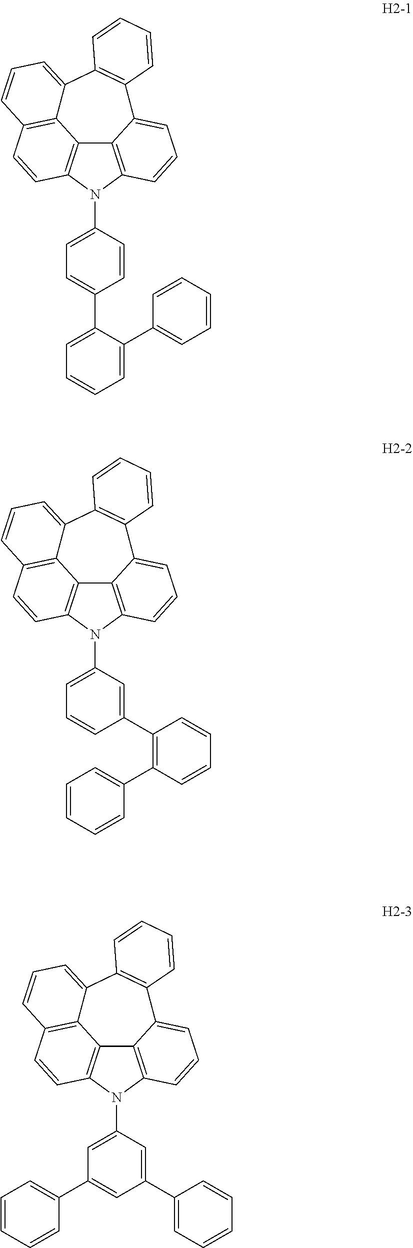 [SMILES: c1ccc(-c2ccccc2-c2ccc(-n3c4cccc5c4c4c6c(cccc6ccc43)-c3ccccc3-5)cc2)cc1 ; c1ccc(-c2ccccc2-c2cccc(-n3c4cccc5c4c4c6c(cccc6ccc43)-c3ccccc3-5)c2)cc1 ; c1ccc(-c2cc(-c3ccccc3)cc(-n3c4cccc5c4c4c6c(cccc6ccc43)-c3ccccc3-5)c2)cc1]

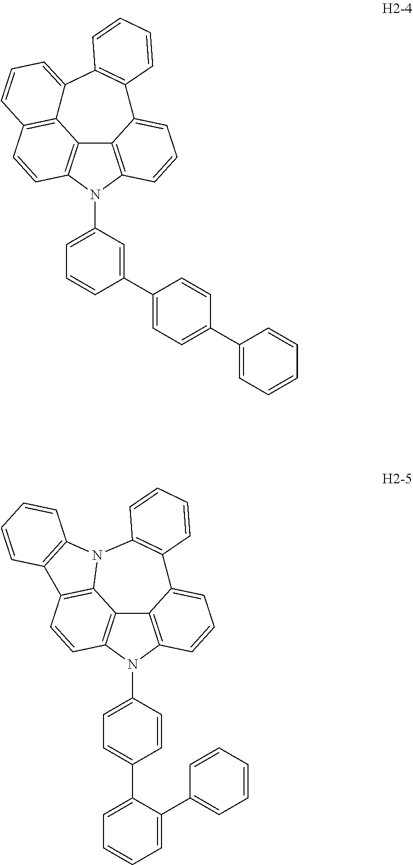 [SMILES: c1ccc(-c2ccc(-c3cccc(-n4c5cccc6c5c5c7c(cccc7ccc54)-c4ccccc4-6)c3)cc2)cc1 ; c1ccc(-c2ccccc2-c2ccc(-n3c4cccc5c6ccccc6n6c7ccccc7c7ccc3c(c54)c76)cc2)cc1]

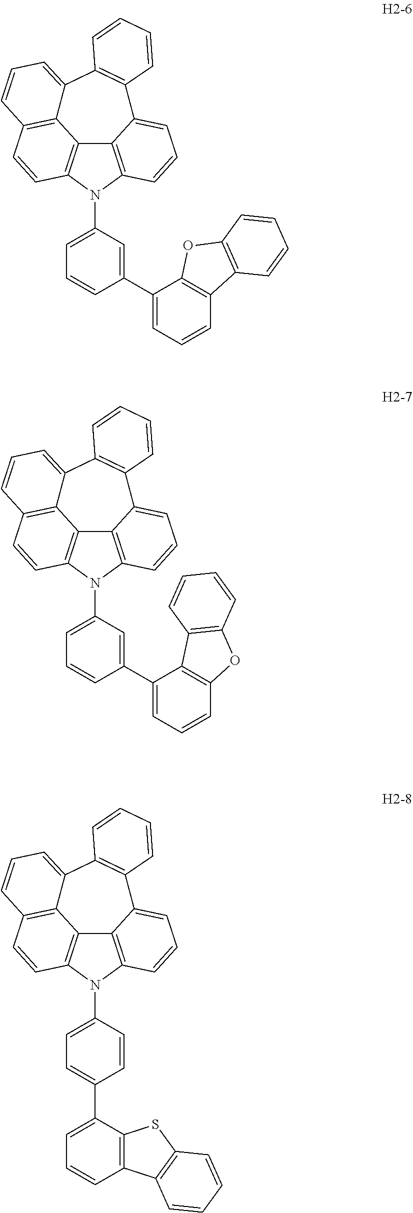 [SMILES: c1cc(-c2cccc3c2oc2ccccc23)cc(-n2c3cccc4c3c3c5c(cccc5ccc32)-c2ccccc2-4)c1 ; c1cc(-c2cccc3oc4ccccc4c23)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc(-c2cccc3c2sc2ccccc23)cc1]

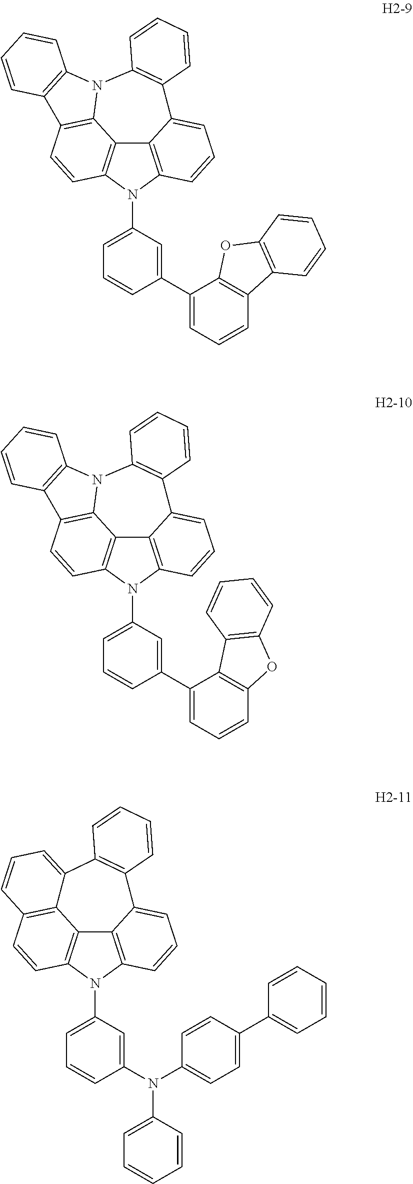 [SMILES: c1cc(-c2cccc3c2oc2ccccc23)cc(-n2c3cccc4c5ccccc5n5c6ccccc6c6ccc2c(c43)c65)c1 ; c1cc(-c2cccc3oc4ccccc4c23)cc(-n2c3cccc4c5ccccc5n5c6ccccc6c6ccc2c(c43)c65)c1 ; c1ccc(-c2ccc(N(c3ccccc3)c3cccc(-n4c5cccc6c5c5c7c(cccc7ccc54)-c4ccccc4-6)c3)cc2)cc1]

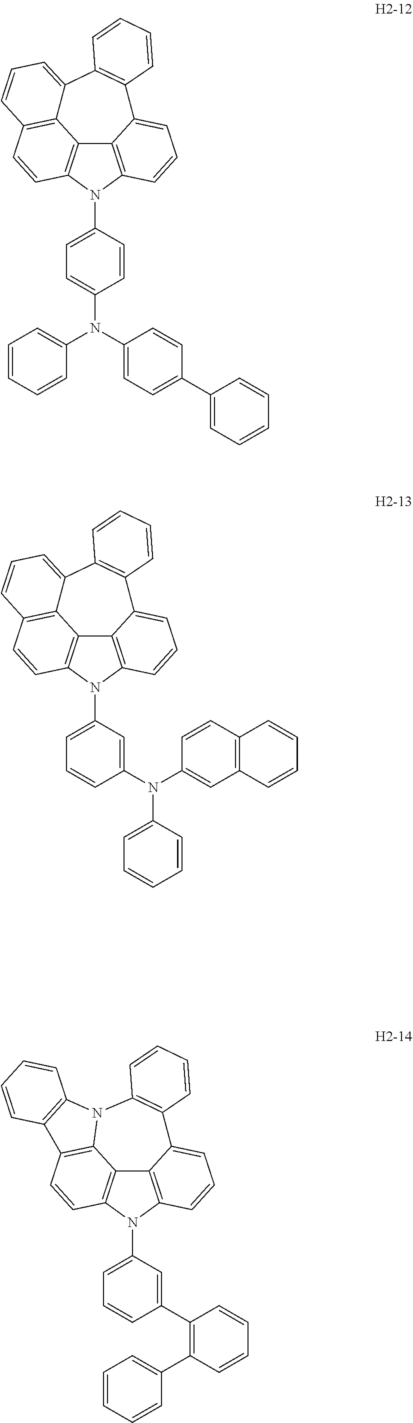 [SMILES: c1ccc(-c2ccc(N(c3ccccc3)c3ccc(-n4c5cccc6c5c5c7c(cccc7ccc54)-c4ccccc4-6)cc3)cc2)cc1 ; c1ccc(N(c2cccc(-n3c4cccc5c4c4c6c(cccc6ccc43)-c3ccccc3-5)c2)c2ccc3ccccc3c2)cc1 ; c1ccc(-c2ccccc2-c2cccc(-n3c4cccc5c6ccccc6n6c7ccccc7c7ccc3c(c54)c76)c2)cc1]

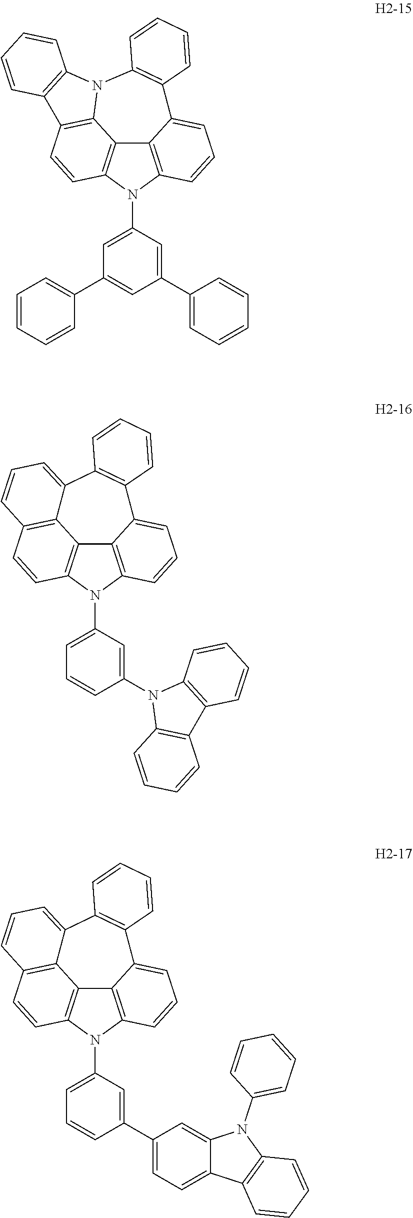 [SMILES: c1ccc(-c2cc(-c3ccccc3)cc(-n3c4cccc5c6ccccc6n6c7ccccc7c7ccc3c(c54)c76)c2)cc1 ; c1cc(-n2c3ccccc3c3ccccc32)cc(-n2c3cccc4c3c3c5c(cccc5ccc32)-c2ccccc2-4)c1 ; c1ccc(-n2c3ccccc3c3ccc(-c4cccc(-n5c6cccc7c6c6c8c(cccc8ccc65)-c5ccccc5-7)c4)cc32)cc1]

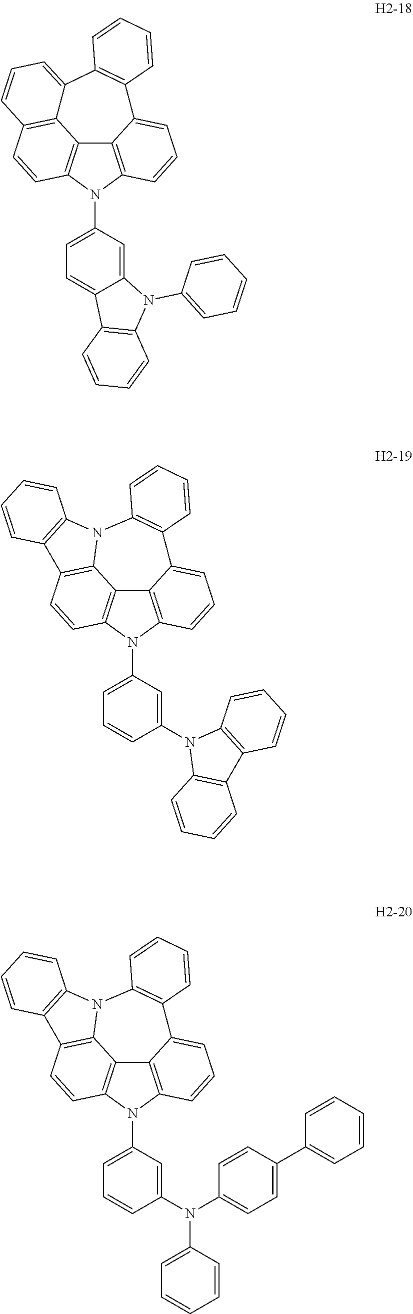 [SMILES: c1ccc(-n2c3ccccc3c3ccc(-n4c5cccc6c5c5c7c(cccc7ccc54)-c4ccccc4-6)cc32)cc1 ; c1cc(-n2c3ccccc3c3ccccc32)cc(-n2c3cccc4c5ccccc5n5c6ccccc6c6ccc2c(c43)c65)c1 ; c1ccc(-c2ccc(N(c3ccccc3)c3cccc(-n4c5cccc6c7ccccc7n7c8ccccc8c8ccc4c(c65)c87)c3)cc2)cc1]

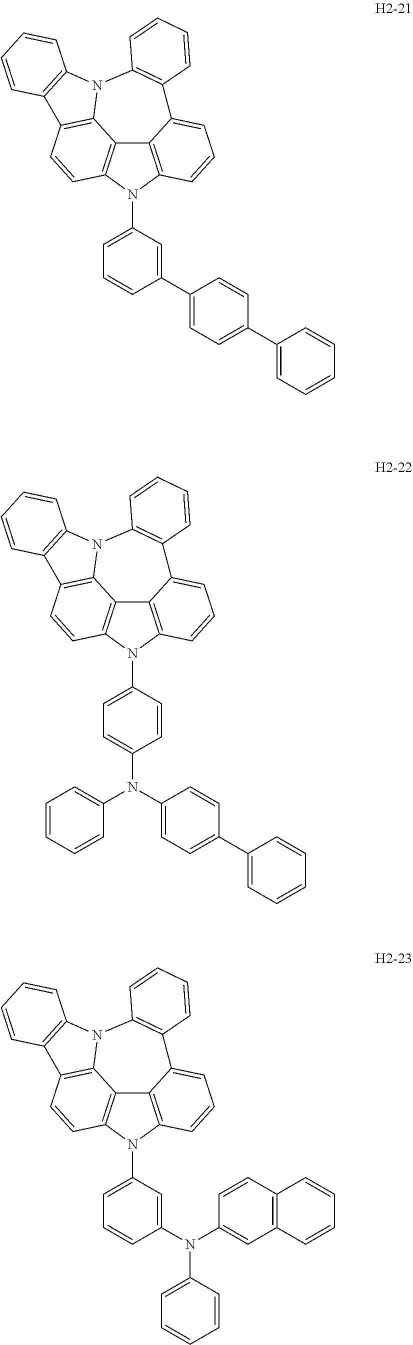 [SMILES: c1ccc(-c2ccc(-c3cccc(-n4c5cccc6c7ccccc7n7c8ccccc8c8ccc4c(c65)c87)c3)cc2)cc1 ; c1ccc(-c2ccc(N(c3ccccc3)c3ccc(-n4c5cccc6c7ccccc7n7c8ccccc8c8ccc4c(c65)c87)cc3)cc2)cc1 ; c1ccc(N(c2cccc(-n3c4cccc5c6ccccc6n6c7ccccc7c7ccc3c(c54)c76)c2)c2ccc3ccccc3c2)cc1]

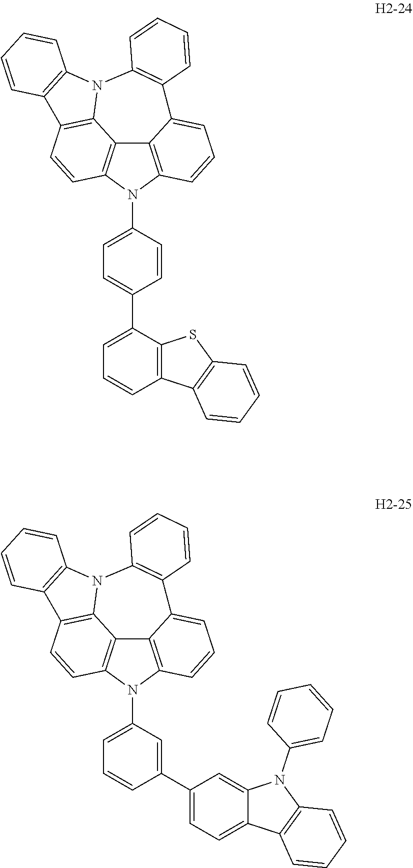 [SMILES: c1ccc2c(c1)sc1c(-c3ccc(-n4c5cccc6c7ccccc7n7c8ccccc8c8ccc4c(c65)c87)cc3)cccc12 ; c1ccc(-n2c3ccccc3c3ccc(-c4cccc(-n5c6cccc7c8ccccc8n8c9ccccc9c9ccc5c(c76)c98)c4)cc32)cc1]

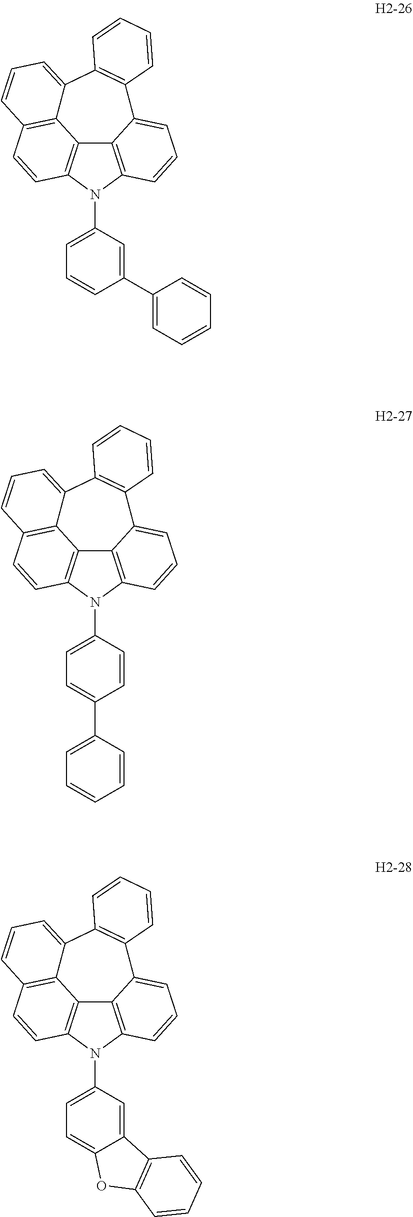 [SMILES: c1ccc(-c2cccc(-n3c4cccc5c4c4c6c(cccc6ccc43)-c3ccccc3-5)c2)cc1 ; c1ccc(-c2ccc(-n3c4cccc5c4c4c6c(cccc6ccc43)-c3ccccc3-5)cc2)cc1 ; c1ccc2c(c1)-c1cccc3ccc4c(c13)c1c-2cccc1n4-c1ccc2oc3ccccc3c2c1]

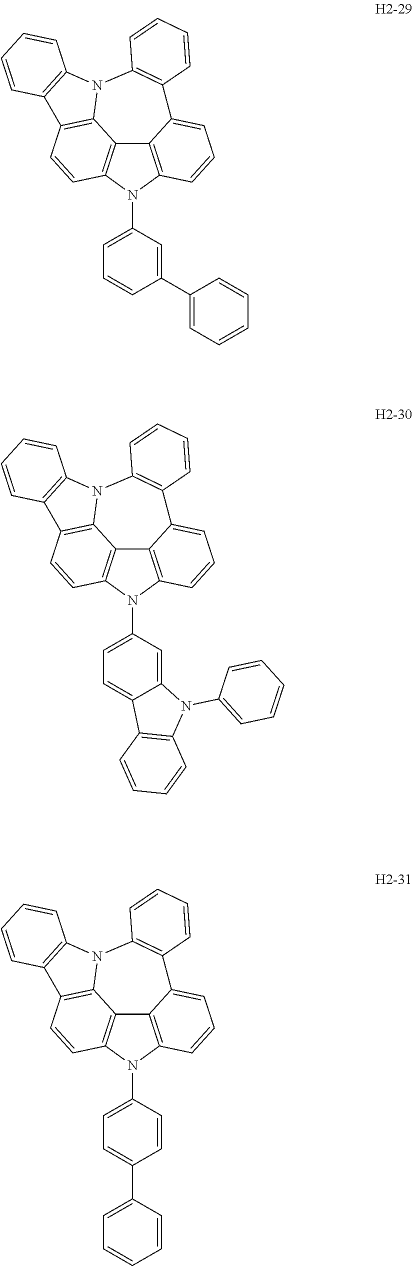 [SMILES: c1ccc(-c2cccc(-n3c4cccc5c6ccccc6n6c7ccccc7c7ccc3c(c54)c76)c2)cc1 ; c1ccc(-n2c3ccccc3c3ccc(-n4c5cccc6c7ccccc7n7c8ccccc8c8ccc4c(c65)c87)cc32)cc1 ; c1ccc(-c2ccc(-n3c4cccc5c6ccccc6n6c7ccccc7c7ccc3c(c54)c76)cc2)cc1]

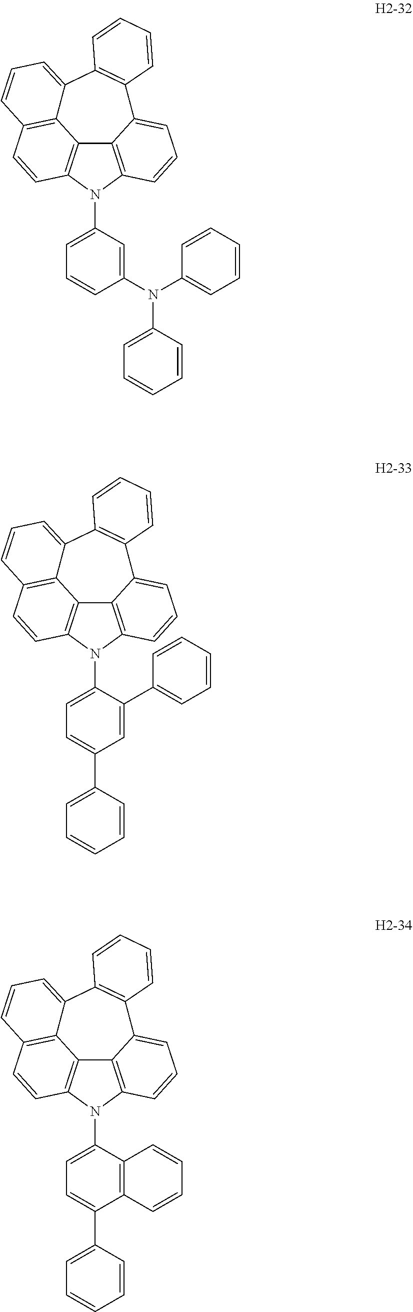 [SMILES: c1ccc(N(c2ccccc2)c2cccc(-n3c4cccc5c4c4c6c(cccc6ccc43)-c3ccccc3-5)c2)cc1 ; c1ccc(-c2ccc(-n3c4cccc5c4c4c6c(cccc6ccc43)-c3ccccc3-5)c(-c3ccccc3)c2)cc1 ; c1ccc(-c2ccc(-n3c4cccc5c4c4c6c(cccc6ccc43)-c3ccccc3-5)c3ccccc23)cc1]

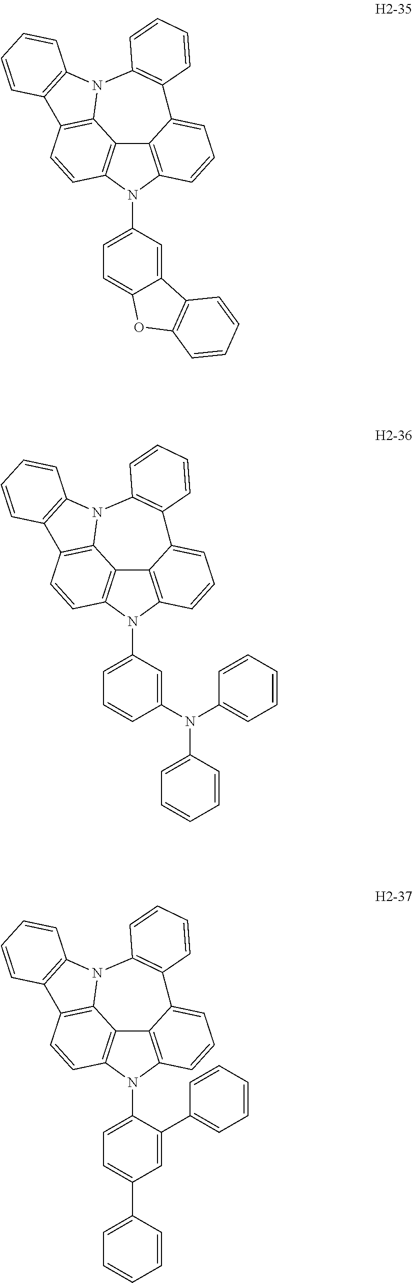 [SMILES: c1ccc2c(c1)oc1ccc(-n3c4cccc5c6ccccc6n6c7ccccc7c7ccc3c(c54)c76)cc12 ; c1ccc(N(c2ccccc2)c2cccc(-n3c4cccc5c6ccccc6n6c7ccccc7c7ccc3c(c54)c76)c2)cc1 ; c1ccc(-c2ccc(-n3c4cccc5c6ccccc6n6c7ccccc7c7ccc3c(c54)c76)c(-c3ccccc3)c2)cc1]

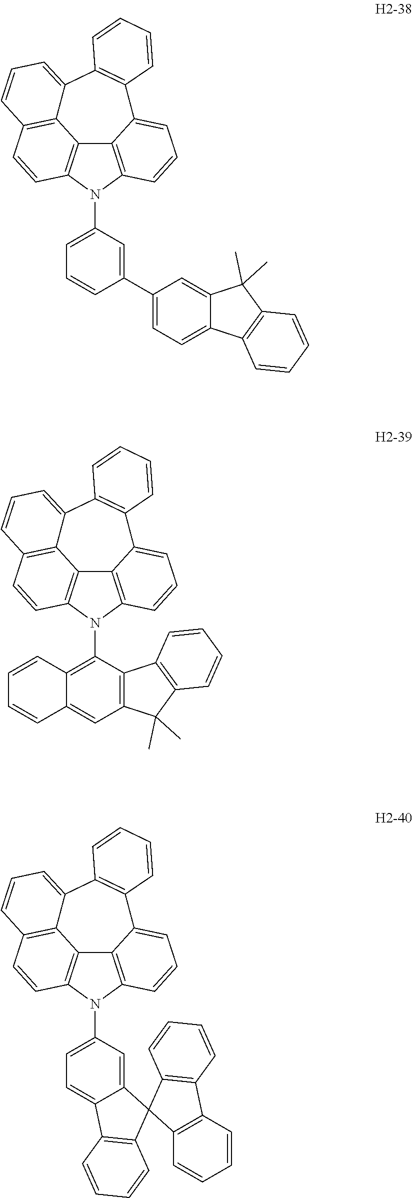 [SMILES: CC1(C)c2ccccc2-c2ccc(-c3cccc(-n4c5cccc6c5c5c7c(cccc7ccc54)-c4ccccc4-6)c3)cc21 ; CC1(C)c2ccccc2-c2c1cc1ccccc1c2-n1c2cccc3c2c2c4c(cccc4ccc21)-c1ccccc1-3 ; c1ccc2c(c1)-c1cccc3ccc4c(c13)c1c-2cccc1n4-c1ccc2c(c1)C1(c3ccccc3-c3ccccc31)c1ccccc1-2]

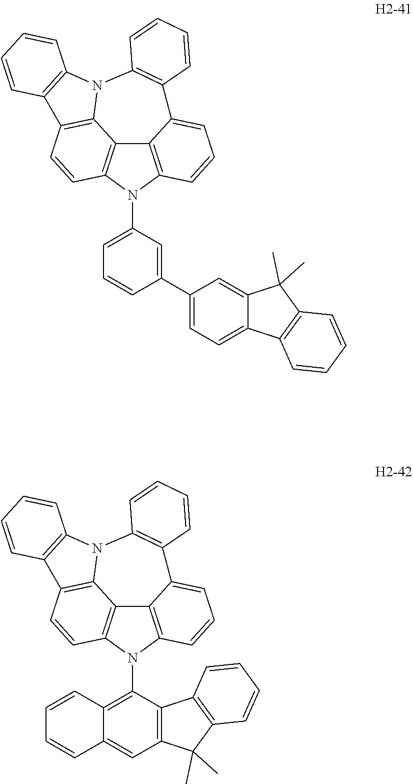 [SMILES: CC1(C)c2ccccc2-c2ccc(-c3cccc(-n4c5cccc6c7ccccc7n7c8ccccc8c8ccc4c(c65)c87)c3)cc21 ; CC1(C)c2ccccc2-c2c1cc1ccccc1c2-n1c2cccc3c4ccccc4n4c5ccccc5c5ccc1c(c32)c54]

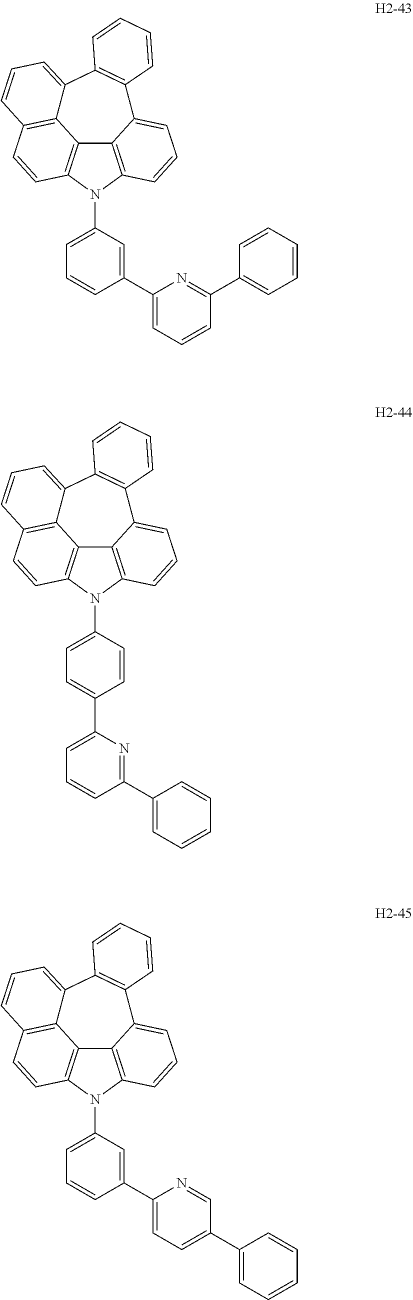 [SMILES: c1ccc(-c2cccc(-c3cccc(-n4c5cccc6c5c5c7c(cccc7ccc54)-c4ccccc4-6)c3)n2)cc1 ; c1ccc(-c2cccc(-c3ccc(-n4c5cccc6c5c5c7c(cccc7ccc54)-c4ccccc4-6)cc3)n2)cc1 ; c1ccc(-c2ccc(-c3cccc(-n4c5cccc6c5c5c7c(cccc7ccc54)-c4ccccc4-6)c3)nc2)cc1]

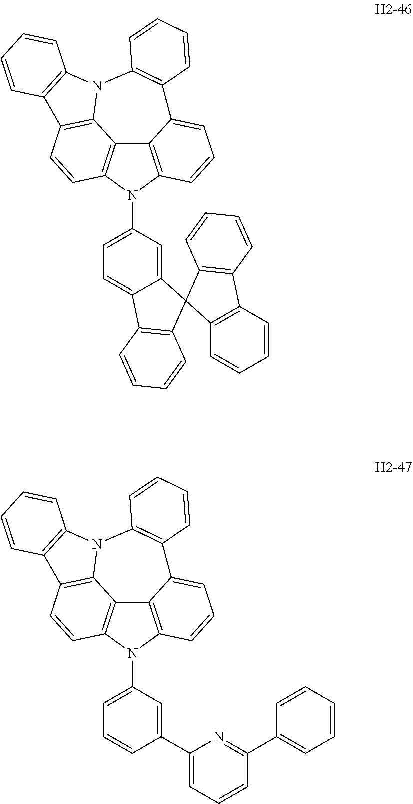 [SMILES: c1ccc2c(c1)-c1ccccc1C21c2ccccc2-c2ccc(-n3c4cccc5c6ccccc6n6c7ccccc7c7ccc3c(c54)c76)cc21 ; c1ccc(-c2cccc(-c3cccc(-n4c5cccc6c7ccccc7n7c8ccccc8c8ccc4c(c65)c87)c3)n2)cc1]

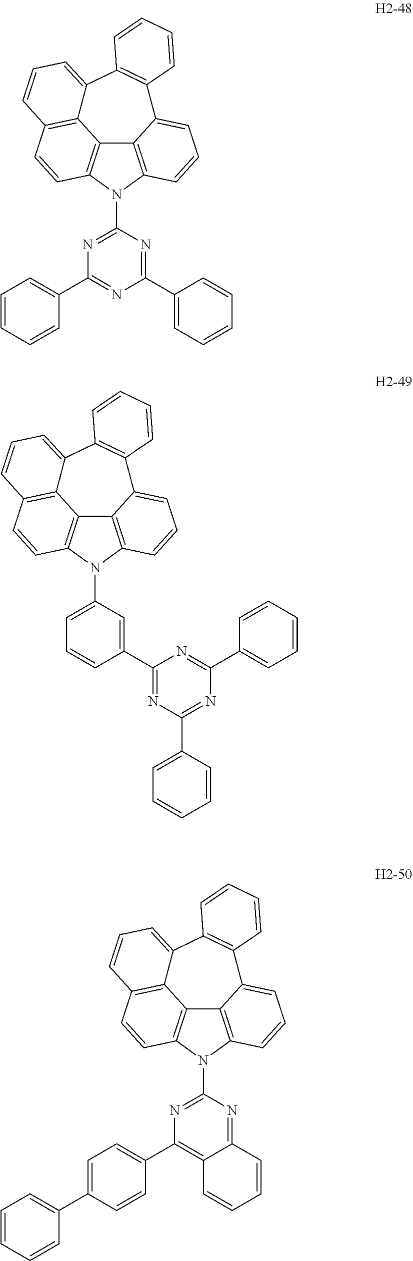 [SMILES: c1ccc(-c2nc(-c3ccccc3)nc(-n3c4cccc5c4c4c6c(cccc6ccc43)-c3ccccc3-5)n2)cc1 ; c1ccc(-c2nc(-c3ccccc3)nc(-c3cccc(-n4c5cccc6c5c5c7c(cccc7ccc54)-c4ccccc4-6)c3)n2)cc1 ; c1ccc(-c2ccc(-c3nc(-n4c5cccc6c5c5c7c(cccc7ccc54)-c4ccccc4-6)nc4ccccc34)cc2)cc1]

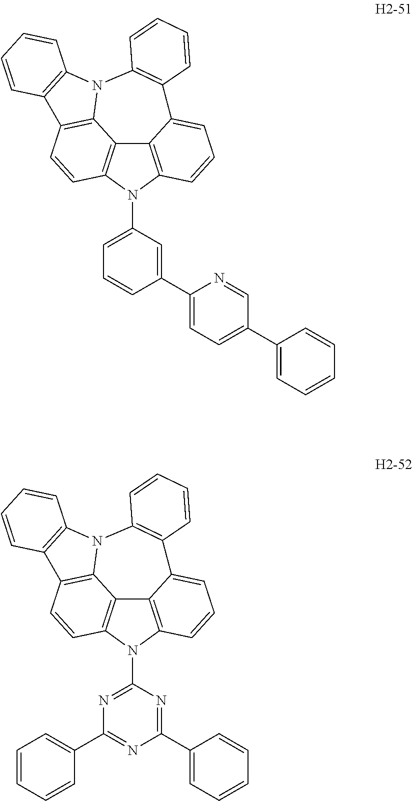 [SMILES: c1ccc(-c2ccc(-c3cccc(-n4c5cccc6c7ccccc7n7c8ccccc8c8ccc4c(c65)c87)c3)nc2)cc1 ; c1ccc(-c2nc(-c3ccccc3)nc(-n3c4cccc5c6ccccc6n6c7ccccc7c7ccc3c(c54)c76)n2)cc1]

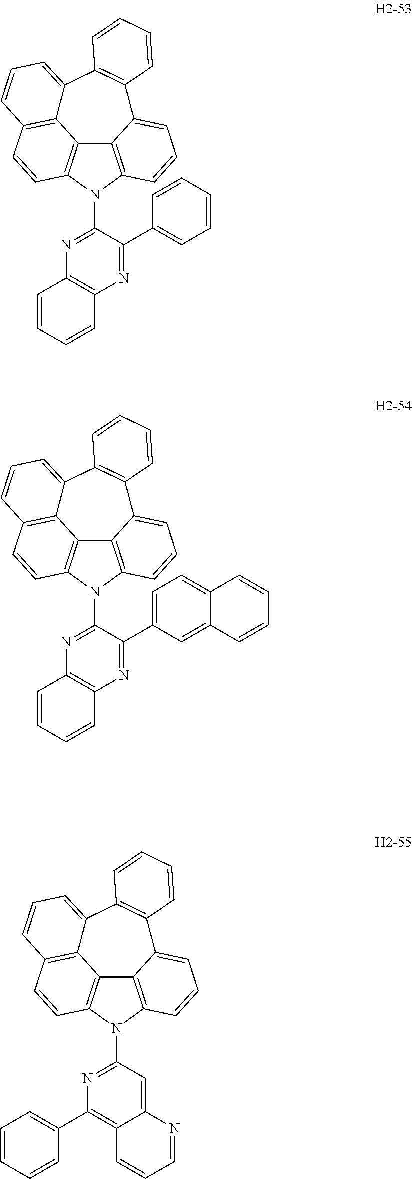 [SMILES: c1ccc(-c2nc3ccccc3nc2-n2c3cccc4c3c3c5c(cccc5ccc32)-c2ccccc2-4)cc1 ; c1ccc2c(c1)-c1cccc3ccc4c(c13)c1c-2cccc1n4-c1nc2ccccc2nc1-c1ccc2ccccc2c1 ; c1ccc(-c2nc(-n3c4cccc5c4c4c6c(cccc6ccc43)-c3ccccc3-5)cc3ncccc23)cc1]

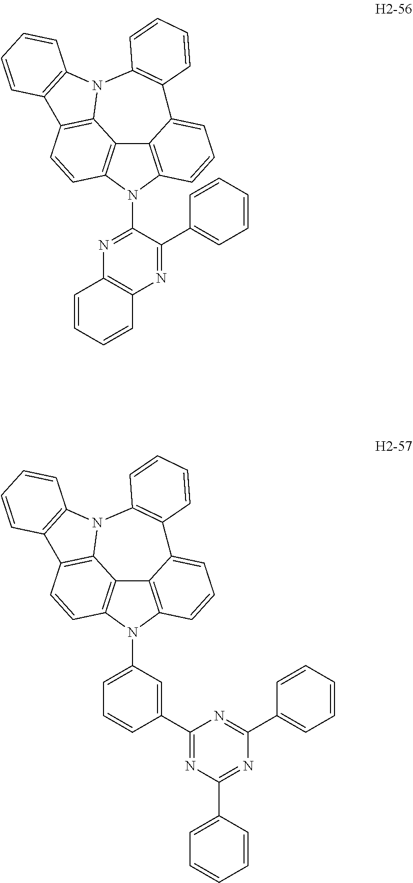 [SMILES: c1ccc(-c2nc3ccccc3nc2-n2c3cccc4c5ccccc5n5c6ccccc6c6ccc2c(c43)c65)cc1 ; c1ccc(-c2nc(-c3ccccc3)nc(-c3cccc(-n4c5cccc6c7ccccc7n7c8ccccc8c8ccc4c(c65)c87)c3)n2)cc1]

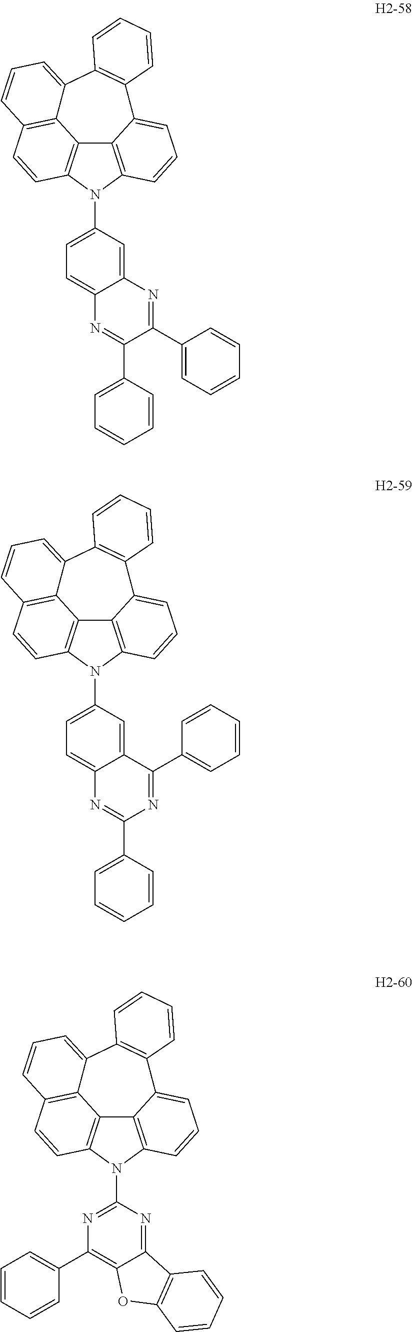 [SMILES: c1ccc(-c2nc3ccc(-n4c5cccc6c5c5c7c(cccc7ccc54)-c4ccccc4-6)cc3nc2-c2ccccc2)cc1 ; c1ccc(-c2nc(-c3ccccc3)c3cc(-n4c5cccc6c5c5c7c(cccc7ccc54)-c4ccccc4-6)ccc3n2)cc1 ; c1ccc(-c2nc(-n3c4cccc5c4c4c6c(cccc6ccc43)-c3ccccc3-5)nc3c2oc2ccccc23)cc1]

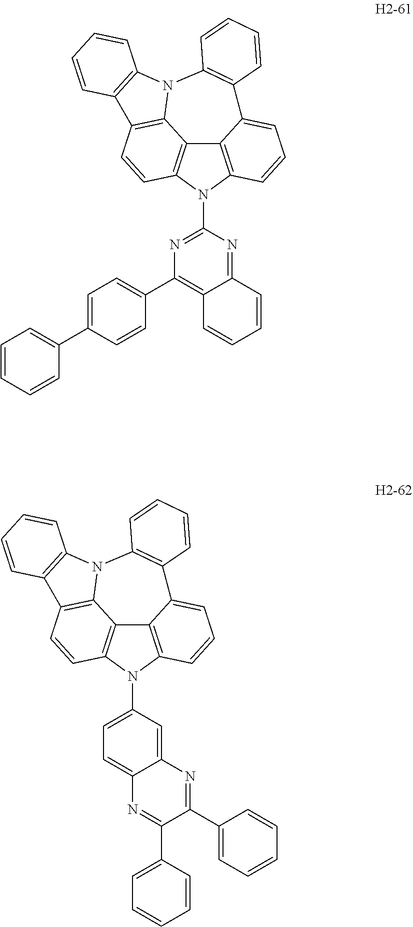 [SMILES: c1ccc(-c2ccc(-c3nc(-n4c5cccc6c7ccccc7n7c8ccccc8c8ccc4c(c65)c87)nc4ccccc34)cc2)cc1 ; c1ccc(-c2nc3ccc(-n4c5cccc6c7ccccc7n7c8ccccc8c8ccc4c(c65)c87)cc3nc2-c2ccccc2)cc1]

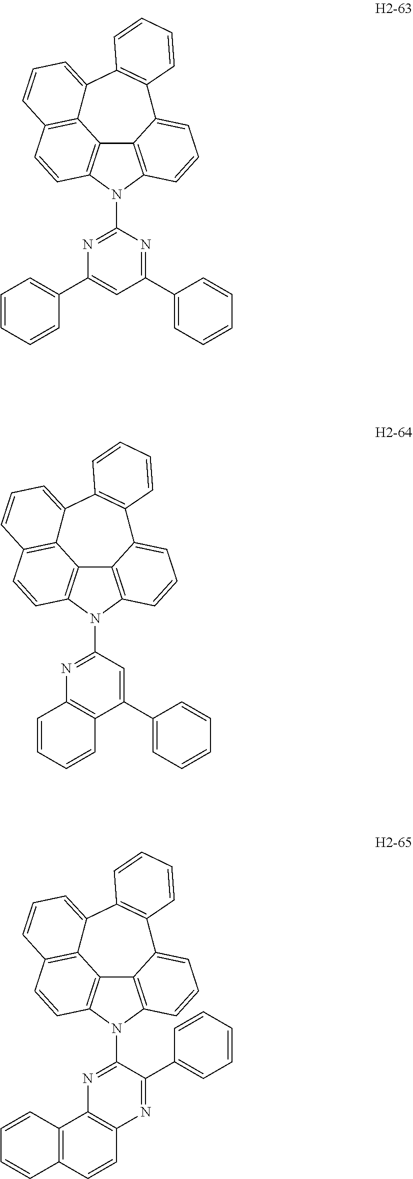 [SMILES: c1ccc(-c2cc(-c3ccccc3)nc(-n3c4cccc5c4c4c6c(cccc6ccc43)-c3ccccc3-5)n2)cc1 ; c1ccc(-c2cc(-n3c4cccc5c4c4c6c(cccc6ccc43)-c3ccccc3-5)nc3ccccc23)cc1 ; c1ccc(-c2nc3ccc4ccccc4c3nc2-n2c3cccc4c3c3c5c(cccc5ccc32)-c2ccccc2-4)cc1]

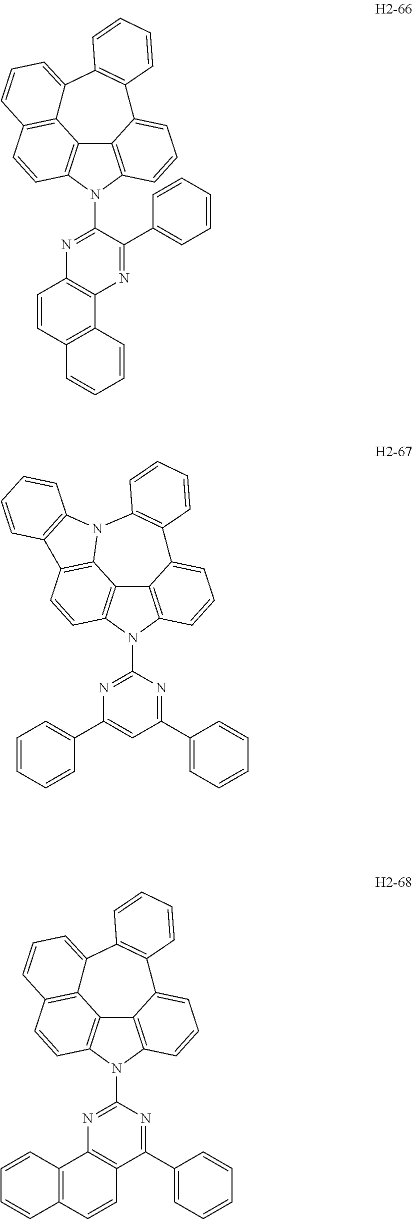 [SMILES: c1ccc(-c2nc3c(ccc4ccccc43)nc2-n2c3cccc4c3c3c5c(cccc5ccc32)-c2ccccc2-4)cc1 ; c1ccc(-c2cc(-c3ccccc3)nc(-n3c4cccc5c6ccccc6n6c7ccccc7c7ccc3c(c54)c76)n2)cc1 ; c1ccc(-c2nc(-n3c4cccc5c4c4c6c(cccc6ccc43)-c3ccccc3-5)nc3c2ccc2ccccc23)cc1]

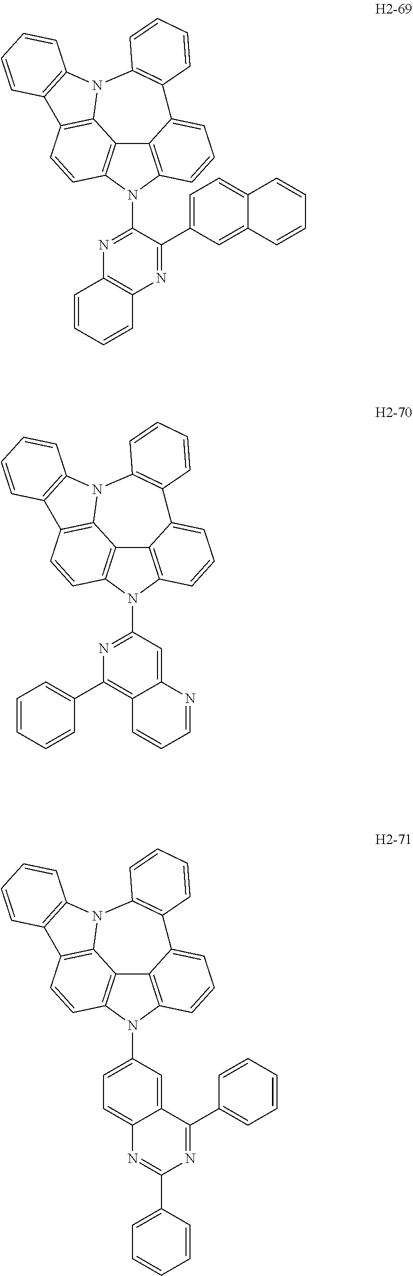 [SMILES: c1ccc2cc(-c3nc4ccccc4nc3-n3c4cccc5c6ccccc6n6c7ccccc7c7ccc3c(c54)c76)ccc2c1 ; c1ccc(-c2nc(-n3c4cccc5c6ccccc6n6c7ccccc7c7ccc3c(c54)c76)cc3ncccc23)cc1 ; c1ccc(-c2nc(-c3ccccc3)c3cc(-n4c5cccc6c7ccccc7n7c8ccccc8c8ccc4c(c65)c87)ccc3n2)cc1]

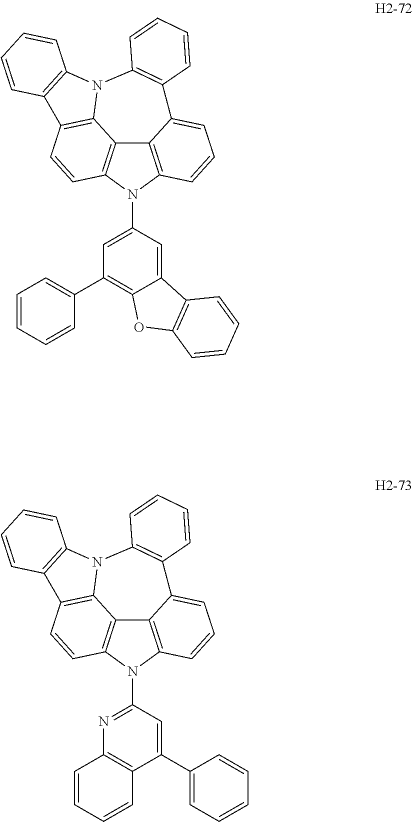 [SMILES: c1ccc(-c2cc(-n3c4cccc5c6ccccc6n6c7ccccc7c7ccc3c(c54)c76)cc3c2oc2ccccc23)cc1 ; c1ccc(-c2cc(-n3c4cccc5c6ccccc6n6c7ccccc7c7ccc3c(c54)c76)nc3ccccc23)cc1]

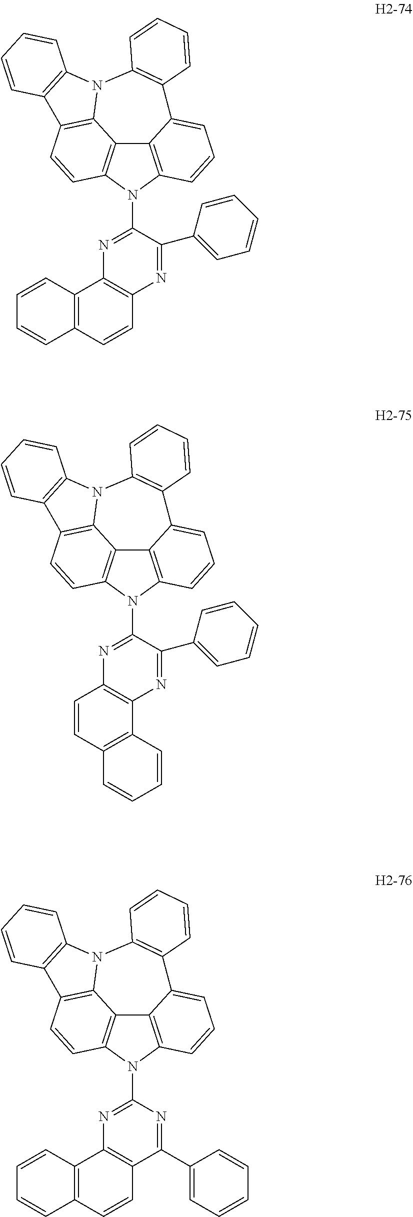 [SMILES: c1ccc(-c2nc3ccc4ccccc4c3nc2-n2c3cccc4c5ccccc5n5c6ccccc6c6ccc2c(c43)c65)cc1 ; c1ccc(-c2nc3c(ccc4ccccc43)nc2-n2c3cccc4c5ccccc5n5c6ccccc6c6ccc2c(c43)c65)cc1 ; c1ccc(-c2nc(-n3c4cccc5c6ccccc6n6c7ccccc7c7ccc3c(c54)c76)nc3c2ccc2ccccc23)cc1]

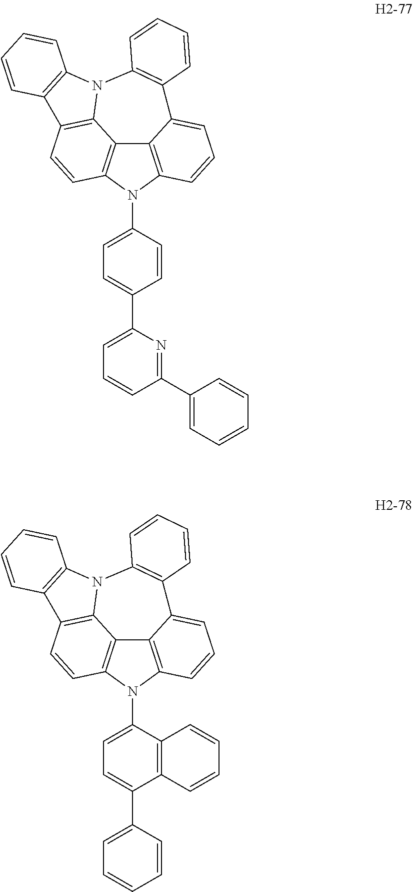 [SMILES: c1ccc(-c2cccc(-c3ccc(-n4c5cccc6c7ccccc7n7c8ccccc8c8ccc4c(c65)c87)cc3)n2)cc1 ; c1ccc(-c2ccc(-n3c4cccc5c6ccccc6n6c7ccccc7c7ccc3c(c54)c76)c3ccccc23)cc1]

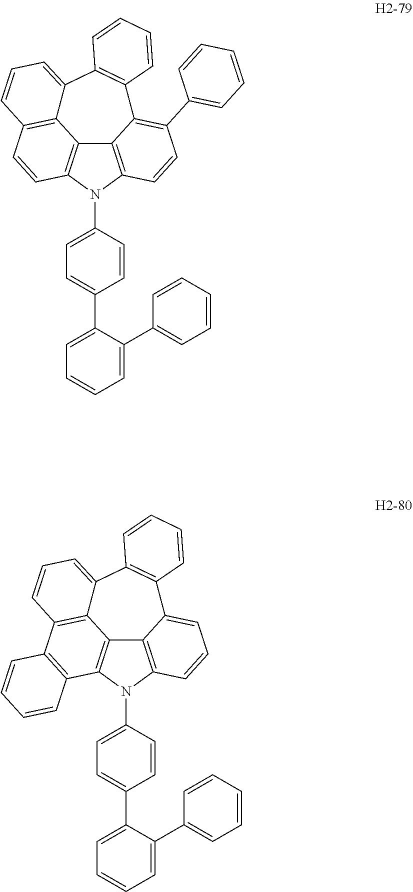 [SMILES: c1ccc(-c2ccccc2-c2ccc(-n3c4ccc(-c5ccccc5)c5c4c4c6c(cccc6ccc43)-c3ccccc3-5)cc2)cc1 ; c1ccc(-c2ccccc2-c2ccc(-n3c4cccc5c4c4c6c(cccc6c6ccccc6c43)-c3ccccc3-5)cc2)cc1]

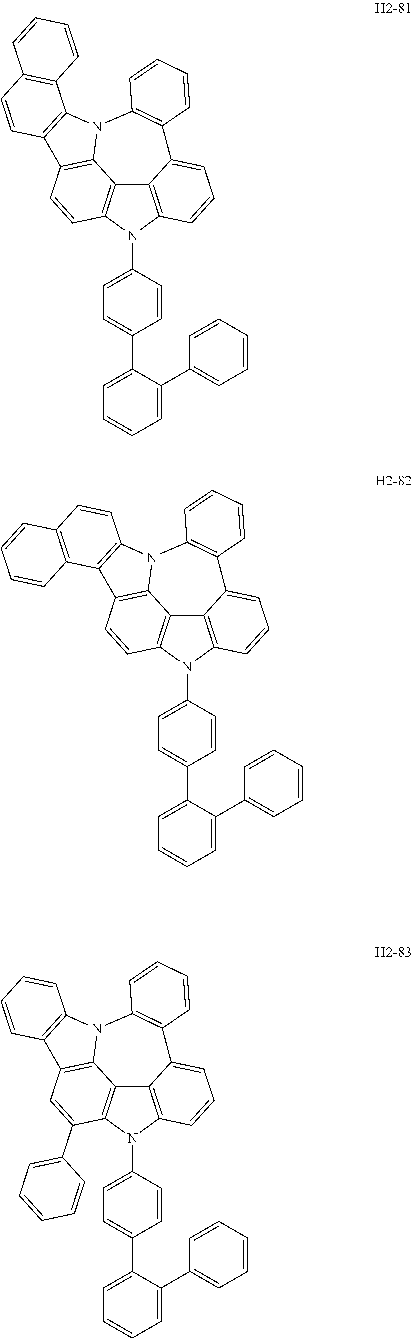 [SMILES: c1ccc(-c2ccccc2-c2ccc(-n3c4cccc5c6ccccc6n6c7c8ccccc8ccc7c7ccc3c(c54)c76)cc2)cc1 ; c1ccc(-c2ccccc2-c2ccc(-n3c4cccc5c6ccccc6n6c7ccc8ccccc8c7c7ccc3c(c54)c76)cc2)cc1 ; c1ccc(-c2ccccc2-c2ccc(-n3c4cccc5c6ccccc6n6c7ccccc7c7cc(-c8ccccc8)c3c(c54)c76)cc2)cc1]

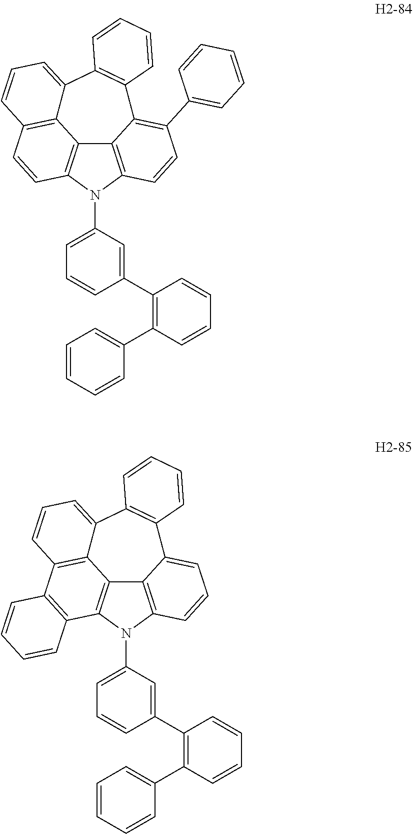 [SMILES: c1ccc(-c2ccccc2-c2cccc(-n3c4ccc(-c5ccccc5)c5c4c4c6c(cccc6ccc43)-c3ccccc3-5)c2)cc1 ; c1ccc(-c2ccccc2-c2cccc(-n3c4cccc5c4c4c6c(cccc6c6ccccc6c43)-c3ccccc3-5)c2)cc1]

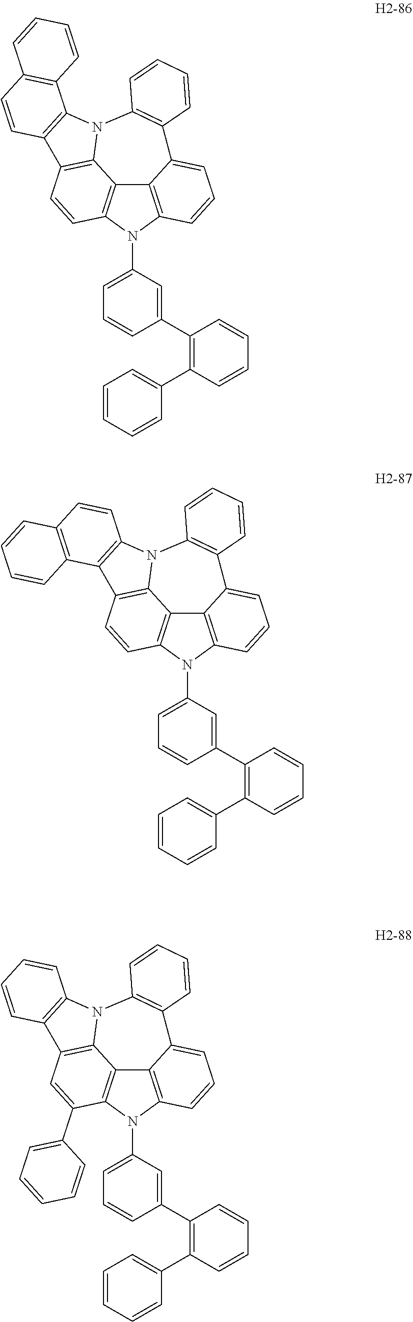 [SMILES: c1ccc(-c2ccccc2-c2cccc(-n3c4cccc5c6ccccc6n6c7c8ccccc8ccc7c7ccc3c(c54)c76)c2)cc1 ; c1ccc(-c2ccccc2-c2cccc(-n3c4cccc5c6ccccc6n6c7ccc8ccccc8c7c7ccc3c(c54)c76)c2)cc1 ; c1ccc(-c2ccccc2-c2cccc(-n3c4cccc5c6ccccc6n6c7ccccc7c7cc(-c8ccccc8)c3c(c54)c76)c2)cc1]

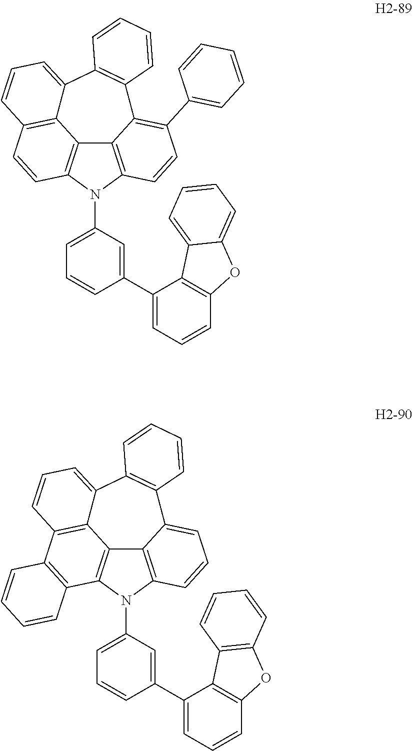 [SMILES: c1ccc(-c2ccc3c4c2-c2ccccc2-c2cccc5ccc(c4c25)n3-c2cccc(-c3cccc4oc5ccccc5c34)c2)cc1 ; c1cc(-c2cccc3oc4ccccc4c23)cc(-n2c3cccc4c3c3c5c(cccc5c5ccccc5c32)-c2ccccc2-4)c1]

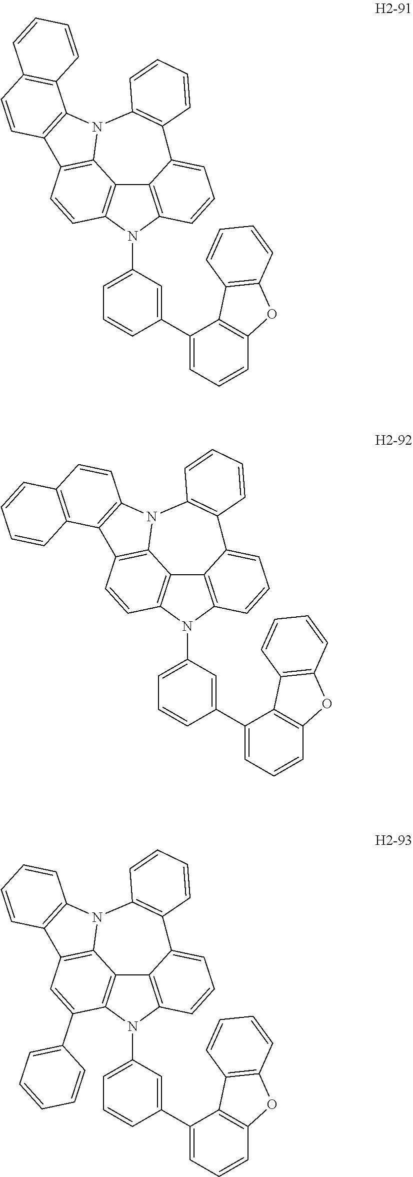 [SMILES: c1cc(-c2cccc3oc4ccccc4c23)cc(-n2c3cccc4c5ccccc5n5c6c7ccccc7ccc6c6ccc2c(c43)c65)c1 ; c1cc(-c2cccc3oc4ccccc4c23)cc(-n2c3cccc4c5ccccc5n5c6ccc7ccccc7c6c6ccc2c(c43)c65)c1 ; c1ccc(-c2cc3c4ccccc4n4c5ccccc5c5cccc6c5c(c2n6-c2cccc(-c5cccc6oc7ccccc7c56)c2)c34)cc1]

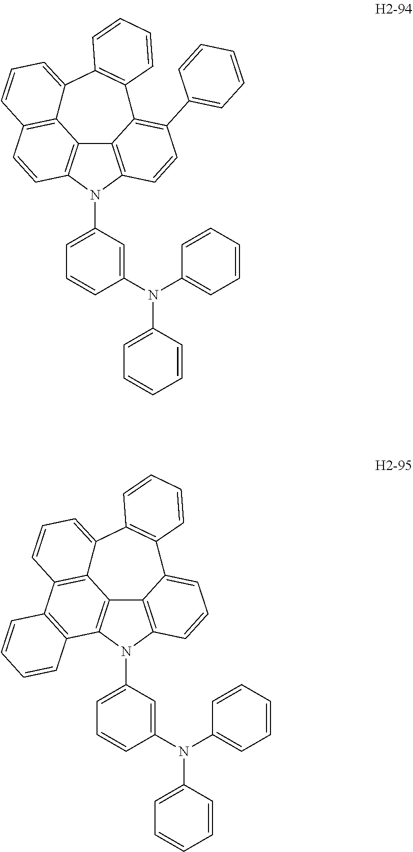 [SMILES: c1ccc(-c2ccc3c4c2-c2ccccc2-c2cccc5ccc(c4c25)n3-c2cccc(N(c3ccccc3)c3ccccc3)c2)cc1 ; c1ccc(N(c2ccccc2)c2cccc(-n3c4cccc5c4c4c6c(cccc6c6ccccc6c43)-c3ccccc3-5)c2)cc1]

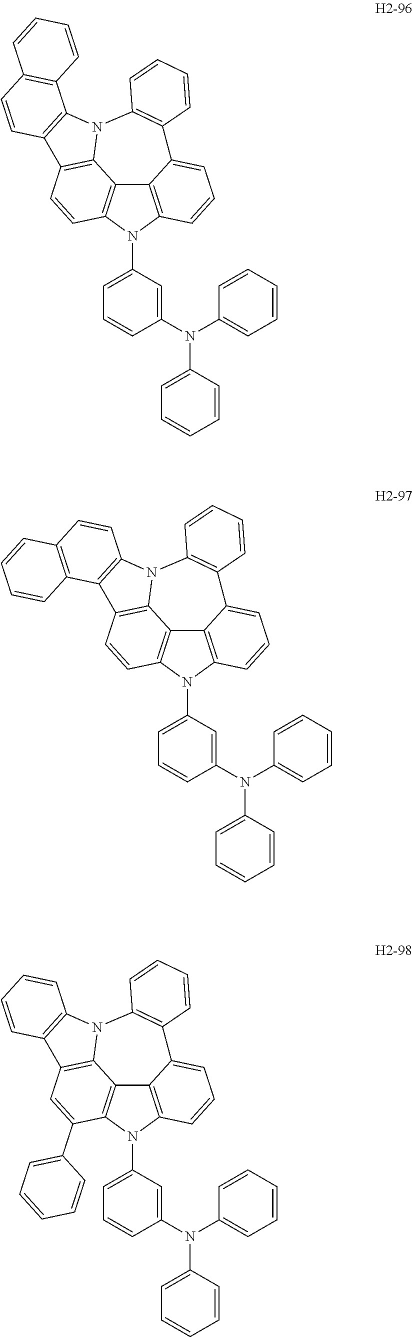 [SMILES: c1ccc(N(c2ccccc2)c2cccc(-n3c4cccc5c6ccccc6n6c7c8ccccc8ccc7c7ccc3c(c54)c76)c2)cc1 ; c1ccc(N(c2ccccc2)c2cccc(-n3c4cccc5c6ccccc6n6c7ccc8ccccc8c7c7ccc3c(c54)c76)c2)cc1 ; c1ccc(-c2cc3c4ccccc4n4c5ccccc5c5cccc6c5c(c2n6-c2cccc(N(c5ccccc5)c5ccccc5)c2)c34)cc1]

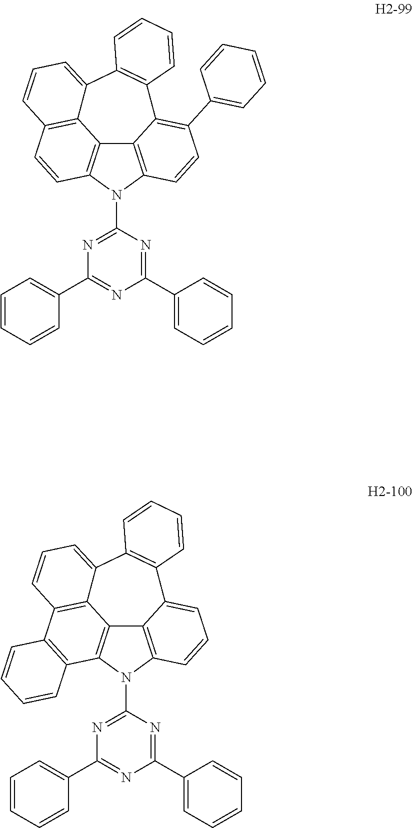 [SMILES: c1ccc(-c2nc(-c3ccccc3)nc(-n3c4ccc(-c5ccccc5)c5c4c4c6c(cccc6ccc43)-c3ccccc3-5)n2)cc1 ; c1ccc(-c2nc(-c3ccccc3)nc(-n3c4cccc5c4c4c6c(cccc6c6ccccc6c43)-c3ccccc3-5)n2)cc1]

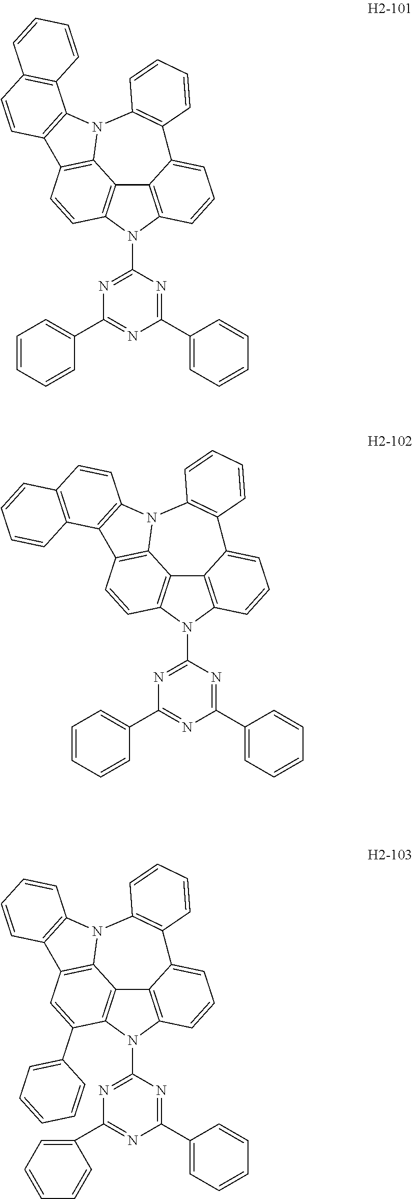 [SMILES: c1ccc(-c2nc(-c3ccccc3)nc(-n3c4cccc5c6ccccc6n6c7c8ccccc8ccc7c7ccc3c(c54)c76)n2)cc1 ; c1ccc(-c2nc(-c3ccccc3)nc(-n3c4cccc5c6ccccc6n6c7ccc8ccccc8c7c7ccc3c(c54)c76)n2)cc1 ; c1ccc(-c2nc(-c3ccccc3)nc(-n3c4cccc5c6ccccc6n6c7ccccc7c7cc(-c8ccccc8)c3c(c54)c76)n2)cc1]

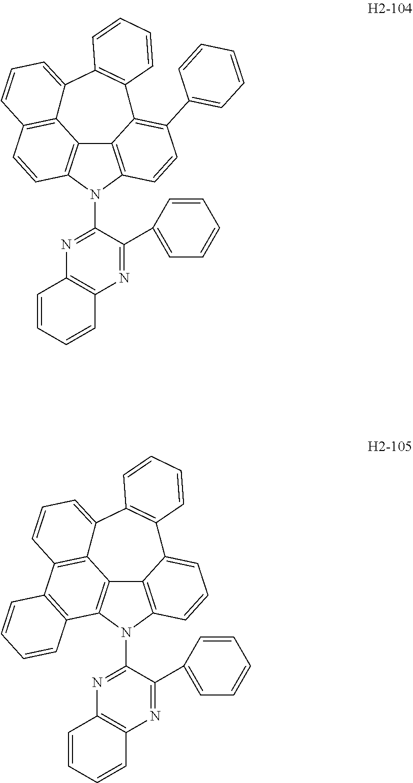 [SMILES: c1ccc(-c2ccc3c4c2-c2ccccc2-c2cccc5ccc(c4c25)n3-c2nc3ccccc3nc2-c2ccccc2)cc1 ; c1ccc(-c2nc3ccccc3nc2-n2c3cccc4c3c3c5c(cccc5c5ccccc5c32)-c2ccccc2-4)cc1]

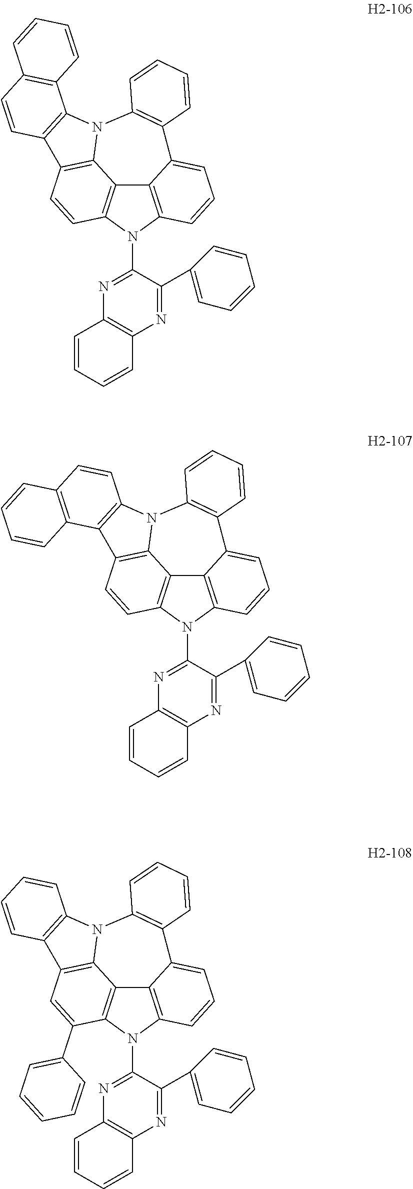 [SMILES: c1ccc(-c2nc3ccccc3nc2-n2c3cccc4c5ccccc5n5c6c7ccccc7ccc6c6ccc2c(c43)c65)cc1 ; c1ccc(-c2nc3ccccc3nc2-n2c3cccc4c5ccccc5n5c6ccc7ccccc7c6c6ccc2c(c43)c65)cc1 ; c1ccc(-c2nc3ccccc3nc2-n2c3cccc4c5ccccc5n5c6ccccc6c6cc(-c7ccccc7)c2c(c43)c65)cc1]

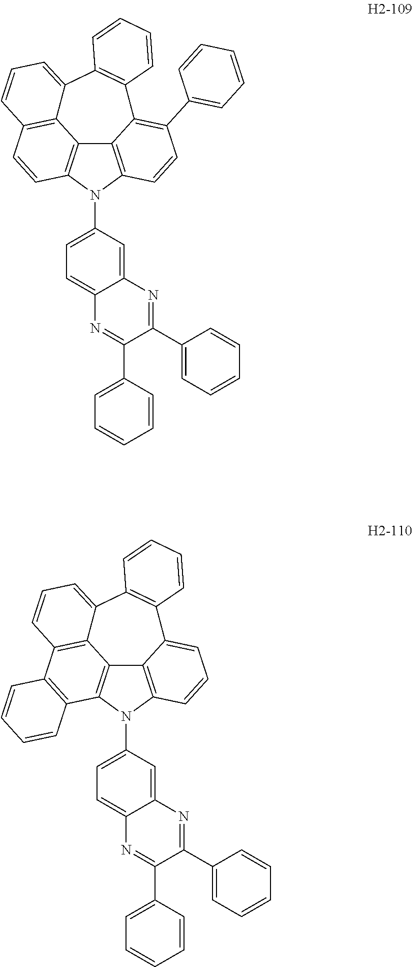 [SMILES: c1ccc(-c2ccc3c4c2-c2ccccc2-c2cccc5ccc(c4c25)n3-c2ccc3nc(-c4ccccc4)c(-c4ccccc4)nc3c2)cc1 ; c1ccc(-c2nc3ccc(-n4c5cccc6c5c5c7c(cccc7c7ccccc7c54)-c4ccccc4-6)cc3nc2-c2ccccc2)cc1]

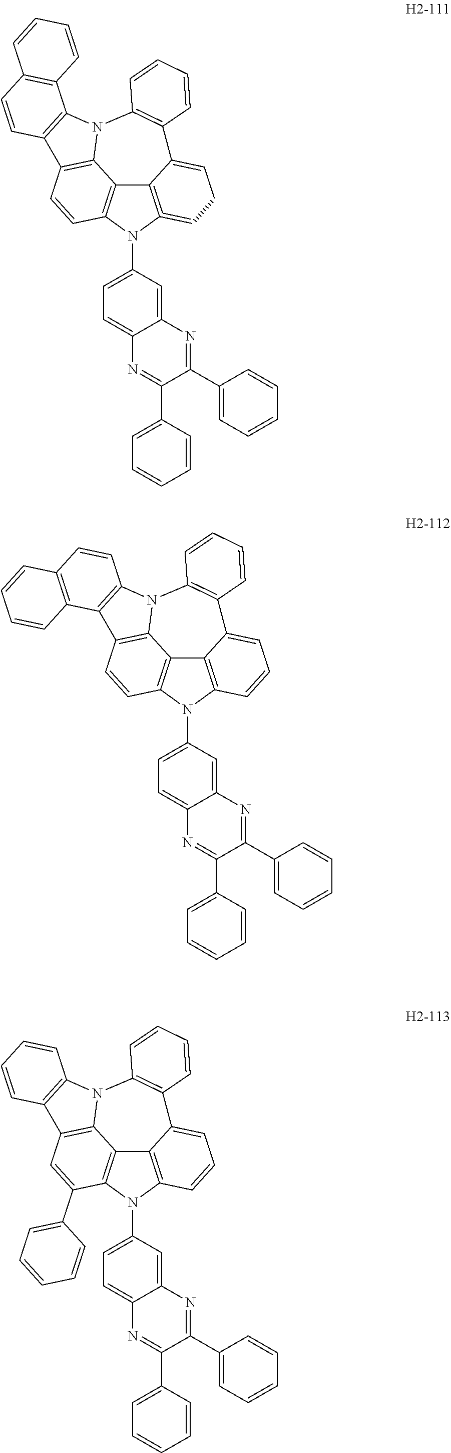

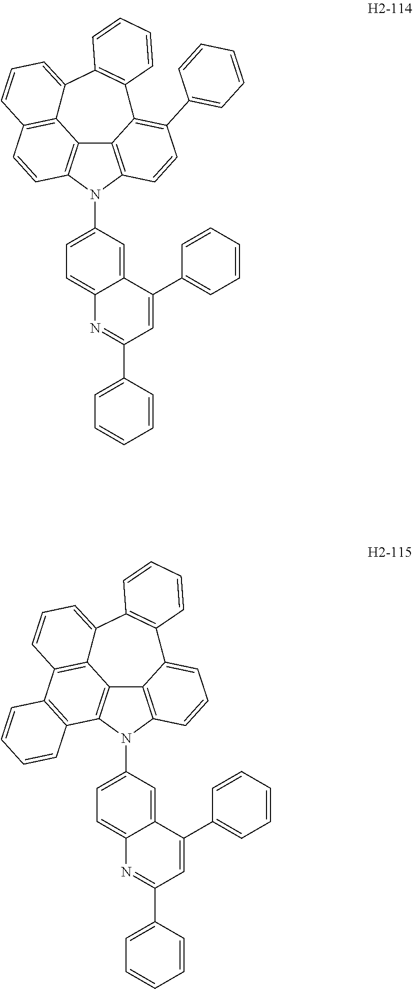 [SMILES: c1ccc(-c2cc(-c3ccccc3)c3cc(-n4c5ccc(-c6ccccc6)c6c5c5c7c(cccc7ccc54)-c4ccccc4-6)ccc3n2)cc1 ; c1ccc(-c2cc(-c3ccccc3)c3cc(-n4c5cccc6c5c5c7c(cccc7c7ccccc7c54)-c4ccccc4-6)ccc3n2)cc1]

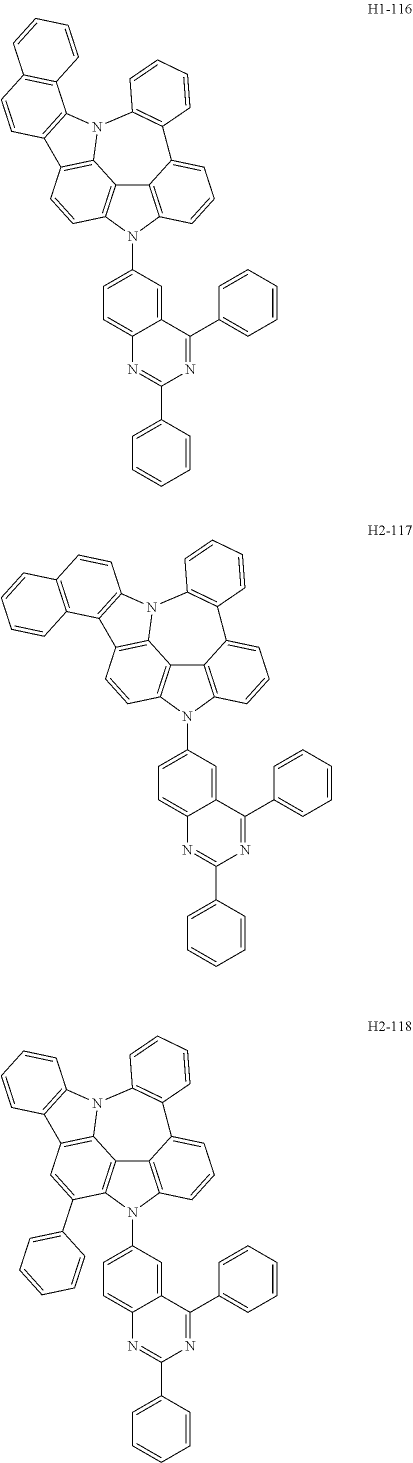 [SMILES: c1ccc(-c2nc(-c3ccccc3)c3cc(-n4c5cccc6c7ccccc7n7c8c9ccccc9ccc8c8ccc4c(c65)c87)ccc3n2)cc1 ; c1ccc(-c2nc(-c3ccccc3)c3cc(-n4c5cccc6c7ccccc7n7c8ccc9ccccc9c8c8ccc4c(c65)c87)ccc3n2)cc1 ; c1ccc(-c2nc(-c3ccccc3)c3cc(-n4c5cccc6c7ccccc7n7c8ccccc8c8cc(-c9ccccc9)c4c(c65)c87)ccc3n2)cc1]

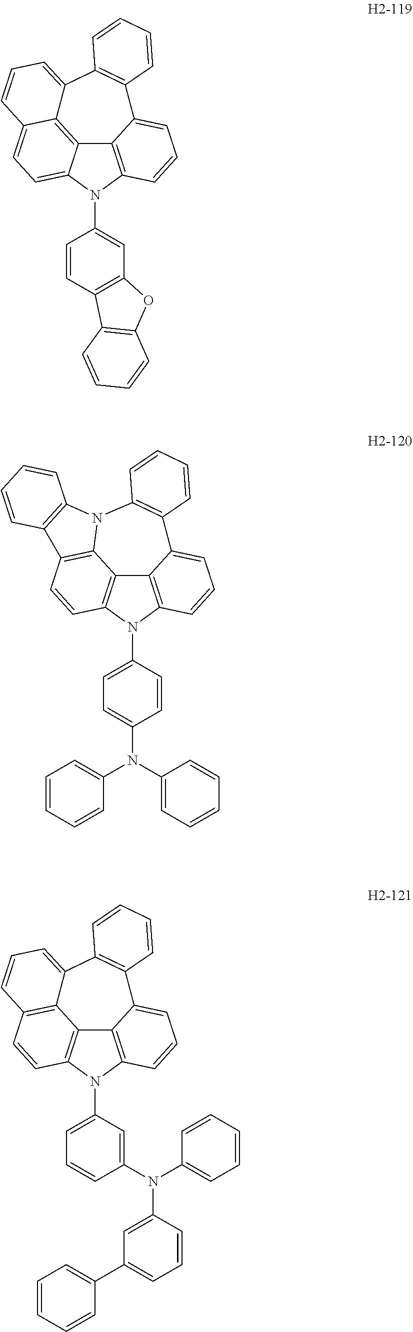 [SMILES: c1ccc2c(c1)-c1cccc3ccc4c(c13)c1c-2cccc1n4-c1ccc2c(c1)oc1ccccc12 ; c1ccc(N(c2ccccc2)c2ccc(-n3c4cccc5c6ccccc6n6c7ccccc7c7ccc3c(c54)c76)cc2)cc1 ; c1ccc(-c2cccc(N(c3ccccc3)c3cccc(-n4c5cccc6c5c5c7c(cccc7ccc54)-c4ccccc4-6)c3)c2)cc1]

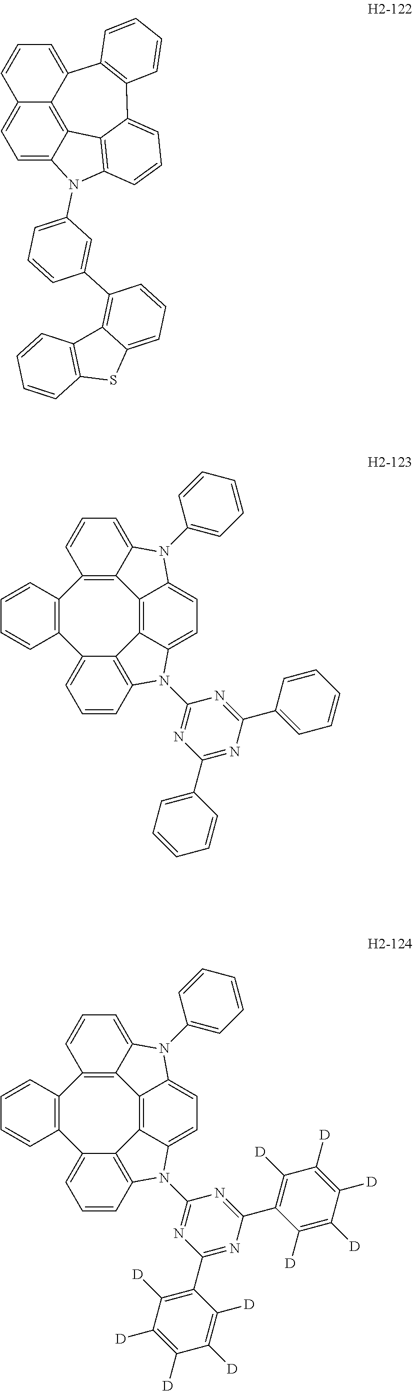 [SMILES: c1cc(-c2cccc3sc4ccccc4c23)cc(-n2c3cccc4c3c3c5c(cccc5ccc32)-c2ccccc2-4)c1 ; c1ccc(-c2nc(-c3ccccc3)nc(-n3c4cccc5c6ccccc6c6cccc7c6c6c(c54)c3ccc6n7-c3ccccc3)n2)cc1 ; [2H]c1c([2H])c([2H])c(-c2nc(-c3c([2H])c([2H])c([2H])c([2H])c3[2H])nc(-n3c4cccc5c6ccccc6c6cccc7c6c6c(c54)c3ccc6n7-c3ccccc3)n2)c([2H])c1[2H]]

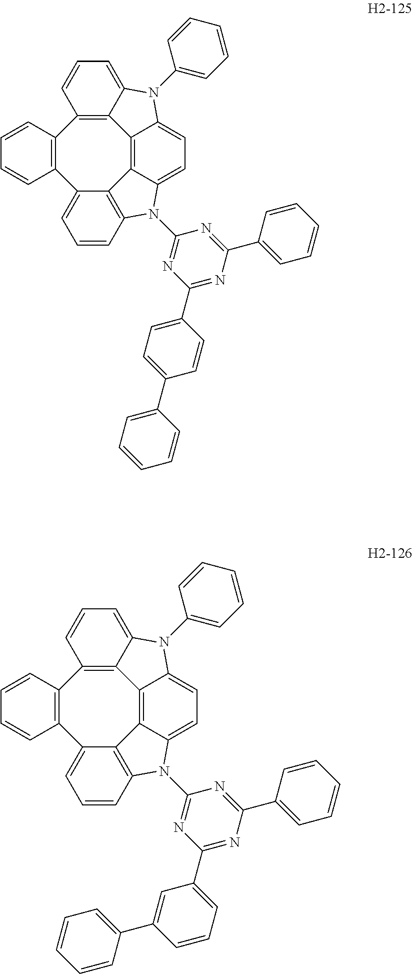 [SMILES: c1ccc(-c2ccc(-c3nc(-c4ccccc4)nc(-n4c5cccc6c7ccccc7c7cccc8c7c7c(c65)c4ccc7n8-c4ccccc4)n3)cc2)cc1 ; c1ccc(-c2cccc(-c3nc(-c4ccccc4)nc(-n4c5cccc6c7ccccc7c7cccc8c7c7c(c65)c4ccc7n8-c4ccccc4)n3)c2)cc1]

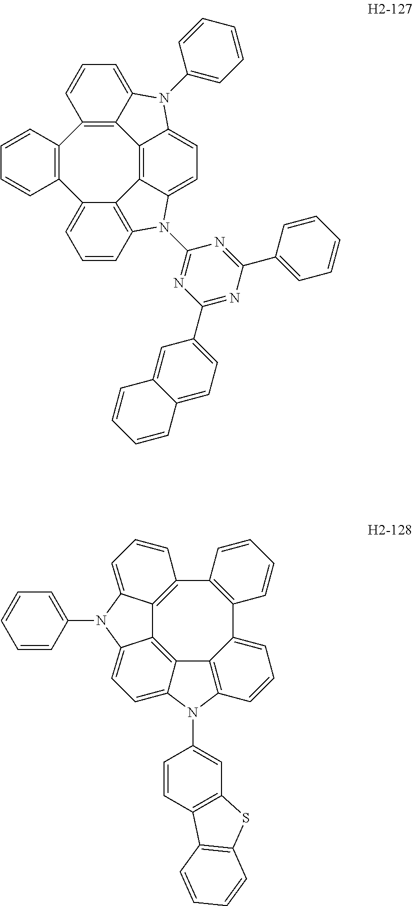 [SMILES: c1ccc(-c2nc(-c3ccc4ccccc4c3)nc(-n3c4cccc5c6ccccc6c6cccc7c6c6c(c54)c3ccc6n7-c3ccccc3)n2)cc1 ; c1ccc(-n2c3cccc4c5ccccc5c5cccc6c5c5c(c43)c2ccc5n6-c2ccc3c(c2)sc2ccccc23)cc1]

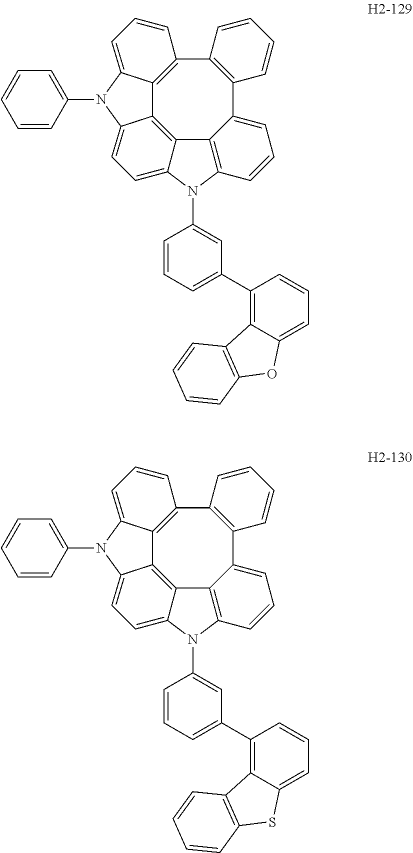 [SMILES: c1ccc(-n2c3cccc4c5ccccc5c5cccc6c5c5c(c43)c2ccc5n6-c2cccc(-c3cccc4oc5ccccc5c34)c2)cc1 ; c1ccc(-n2c3cccc4c5ccccc5c5cccc6c5c5c(c43)c2ccc5n6-c2cccc(-c3cccc4sc5ccccc5c34)c2)cc1]

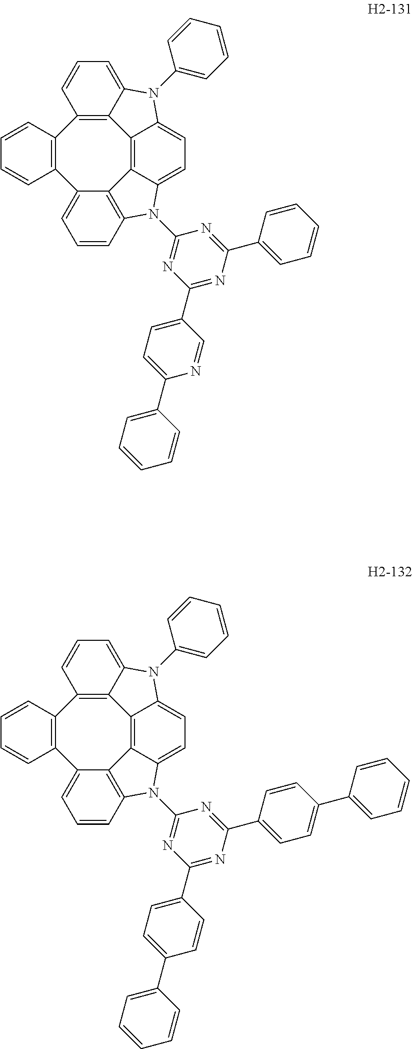 [SMILES: c1ccc(-c2ccc(-c3nc(-c4ccccc4)nc(-n4c5cccc6c7ccccc7c7cccc8c7c7c(c65)c4ccc7n8-c4ccccc4)n3)cn2)cc1 ; c1ccc(-c2ccc(-c3nc(-c4ccc(-c5ccccc5)cc4)nc(-n4c5cccc6c7ccccc7c7cccc8c7c7c(c65)c4ccc7n8-c4ccccc4)n3)cc2)cc1]

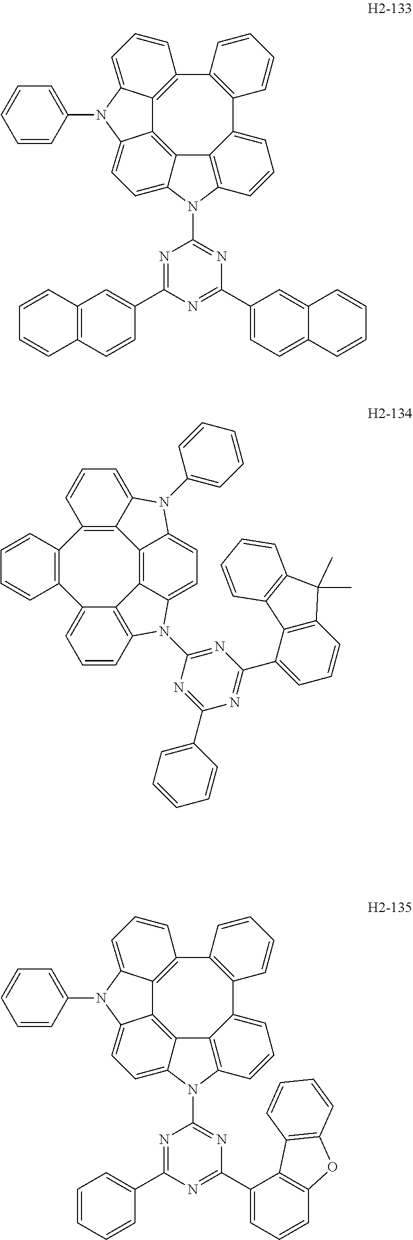 [SMILES: c1ccc(-n2c3cccc4c5ccccc5c5cccc6c5c5c(c43)c2ccc5n6-c2nc(-c3ccc4ccccc4c3)nc(-c3ccc4ccccc4c3)n2)cc1 ; CC1(C)c2ccccc2-c2c(-c3nc(-c4ccccc4)nc(-n4c5cccc6c7ccccc7c7cccc8c7c7c(c65)c4ccc7n8-c4ccccc4)n3)cccc21 ; c1ccc(-c2nc(-c3cccc4oc5ccccc5c34)nc(-n3c4cccc5c6ccccc6c6cccc7c6c6c(c54)c3ccc6n7-c3ccccc3)n2)cc1]

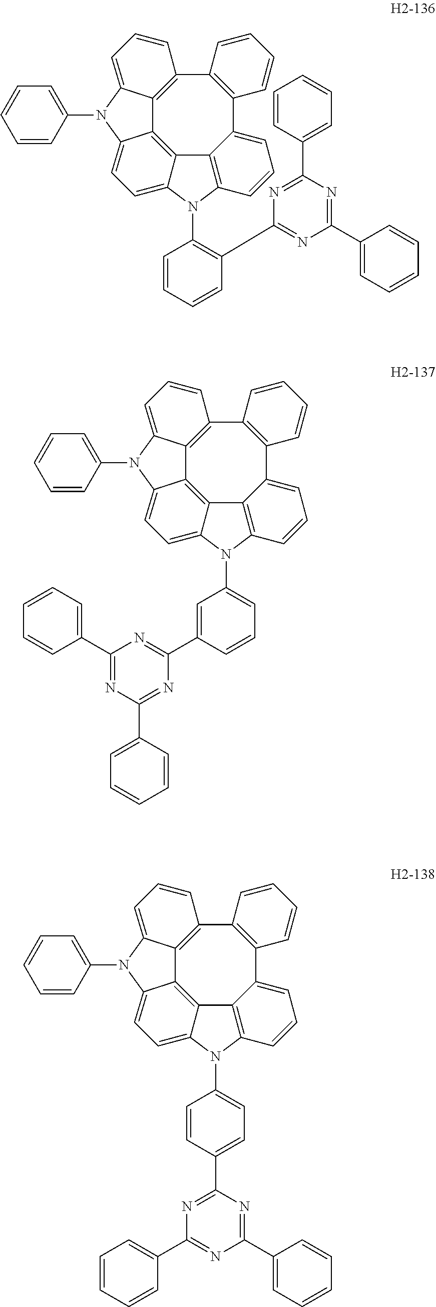 [SMILES: c1ccc(-c2nc(-c3ccccc3)nc(-c3ccccc3-n3c4cccc5c6ccccc6c6cccc7c6c6c(c54)c3ccc6n7-c3ccccc3)n2)cc1 ; c1ccc(-c2nc(-c3ccccc3)nc(-c3cccc(-n4c5cccc6c7ccccc7c7cccc8c7c7c(c65)c4ccc7n8-c4ccccc4)c3)n2)cc1 ; c1ccc(-c2nc(-c3ccccc3)nc(-c3ccc(-n4c5cccc6c7ccccc7c7cccc8c7c7c(c65)c4ccc7n8-c4ccccc4)cc3)n2)cc1]

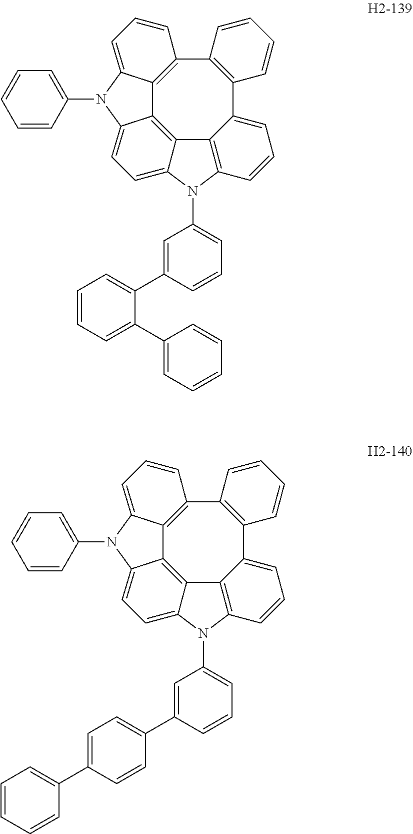 [SMILES: c1ccc(-c2ccccc2-c2cccc(-n3c4cccc5c6ccccc6c6cccc7c6c6c(c54)c3ccc6n7-c3ccccc3)c2)cc1 ; c1ccc(-c2ccc(-c3cccc(-n4c5cccc6c7ccccc7c7cccc8c7c7c(c65)c4ccc7n8-c4ccccc4)c3)cc2)cc1]

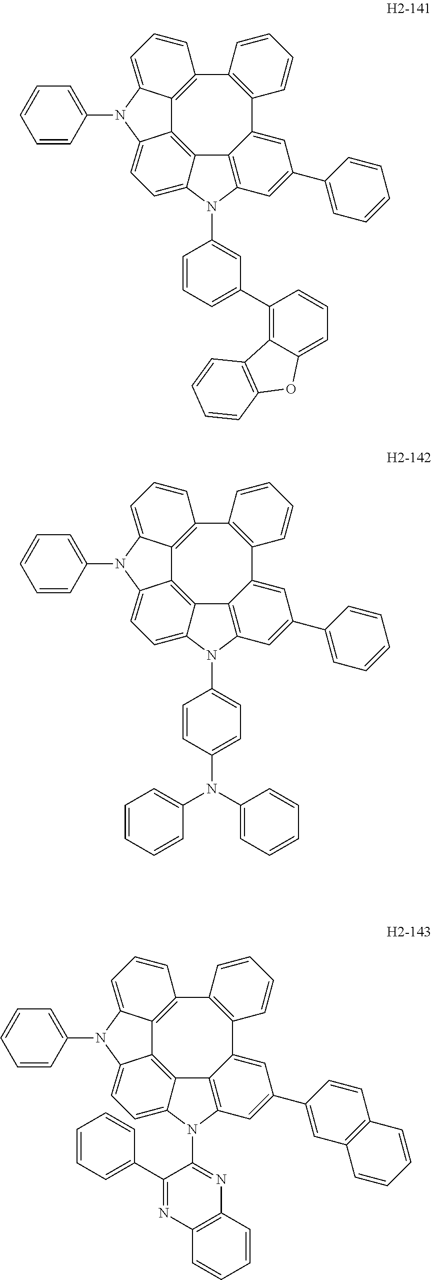 [SMILES: c1ccc(-c2cc3c4ccccc4c4cccc5c4c4c6c3c(c2)n(-c2cccc(-c3cccc7oc8ccccc8c37)c2)c6ccc4n5-c2ccccc2)cc1 ; c1ccc(-c2cc3c4ccccc4c4cccc5c4c4c6c3c(c2)n(-c2ccc(N(c3ccccc3)c3ccccc3)cc2)c6ccc4n5-c2ccccc2)cc1 ; c1ccc(-c2nc3ccccc3nc2-n2c3cc(-c4ccc5ccccc5c4)cc4c5ccccc5c5cccc6c5c5c(c43)c2ccc5n6-c2ccccc2)cc1]

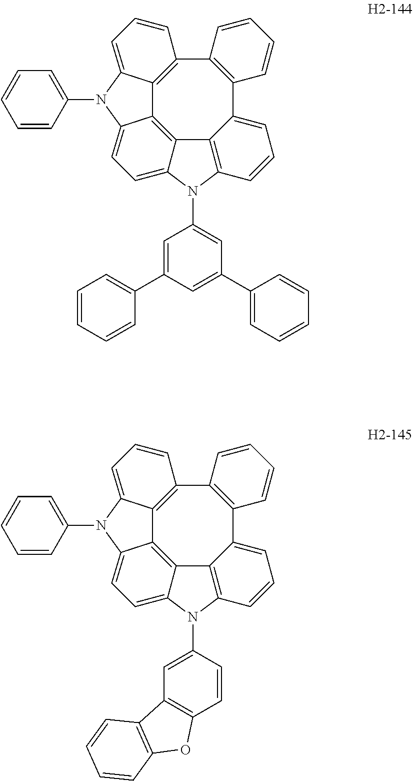 [SMILES: c1ccc(-c2cc(-c3ccccc3)cc(-n3c4cccc5c6ccccc6c6cccc7c6c6c(c54)c3ccc6n7-c3ccccc3)c2)cc1 ; c1ccc(-n2c3cccc4c5ccccc5c5cccc6c5c5c(c43)c2ccc5n6-c2ccc3oc4ccccc4c3c2)cc1]

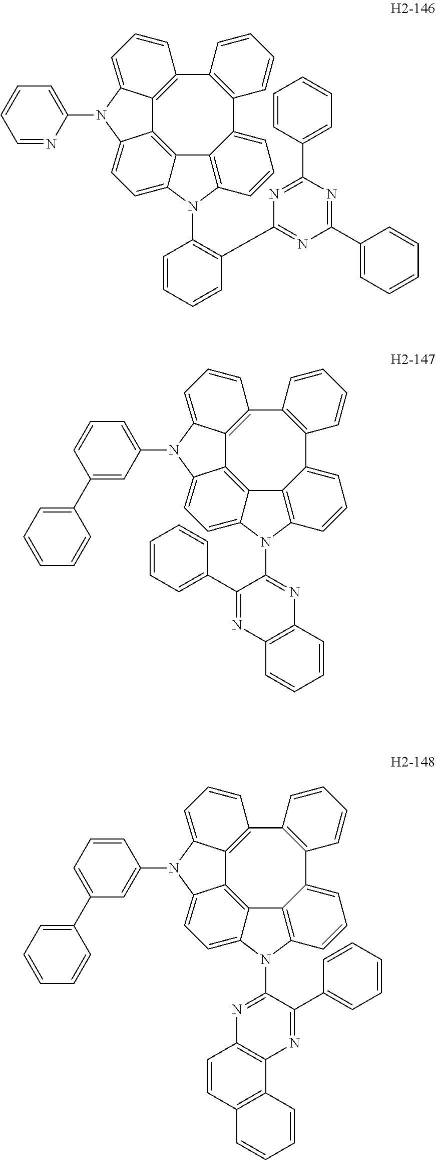 [SMILES: c1ccc(-c2nc(-c3ccccc3)nc(-c3ccccc3-n3c4cccc5c6ccccc6c6cccc7c6c6c(c54)c3ccc6n7-c3ccccn3)n2)cc1 ; c1ccc(-c2cccc(-n3c4cccc5c6ccccc6c6cccc7c6c6c(c54)c3ccc6n7-c3nc4ccccc4nc3-c3ccccc3)c2)cc1 ; c1ccc(-c2cccc(-n3c4cccc5c6ccccc6c6cccc7c6c6c(c54)c3ccc6n7-c3nc4ccc5ccccc5c4nc3-c3ccccc3)c2)cc1]

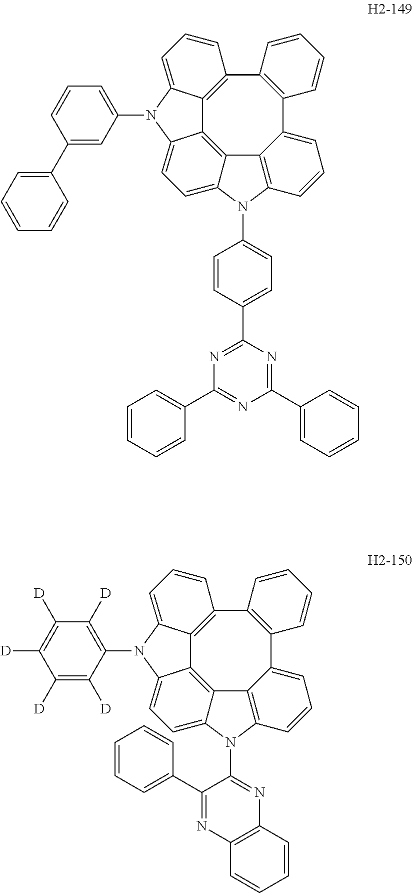 [SMILES: c1ccc(-c2cccc(-n3c4cccc5c6ccccc6c6cccc7c6c6c(c54)c3ccc6n7-c3ccc(-c4nc(-c5ccccc5)nc(-c5ccccc5)n4)cc3)c2)cc1 ; [2H]c1c([2H])c([2H])c(-n2c3cccc4c5ccccc5c5cccc6c5c5c(c43)c2ccc5n6-c2nc3ccccc3nc2-c2ccccc2)c([2H])c1[2H]]

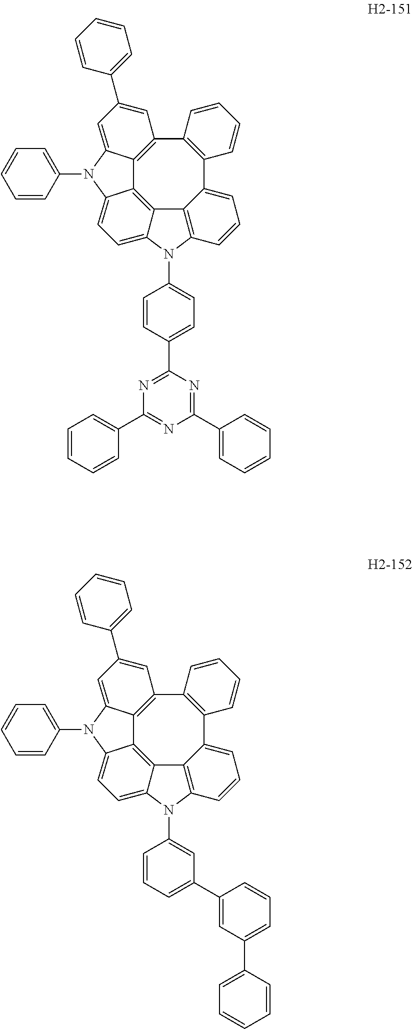 [SMILES: c1ccc(-c2cc3c4ccccc4c4cccc5c4c4c6c3c(c2)n(-c2ccccc2)c6ccc4n5-c2ccc(-c3nc(-c4ccccc4)nc(-c4ccccc4)n3)cc2)cc1 ; c1ccc(-c2cccc(-c3cccc(-n4c5cccc6c7ccccc7c7cc(-c8ccccc8)cc8c7c7c(c65)c4ccc7n8-c4ccccc4)c3)c2)cc1]

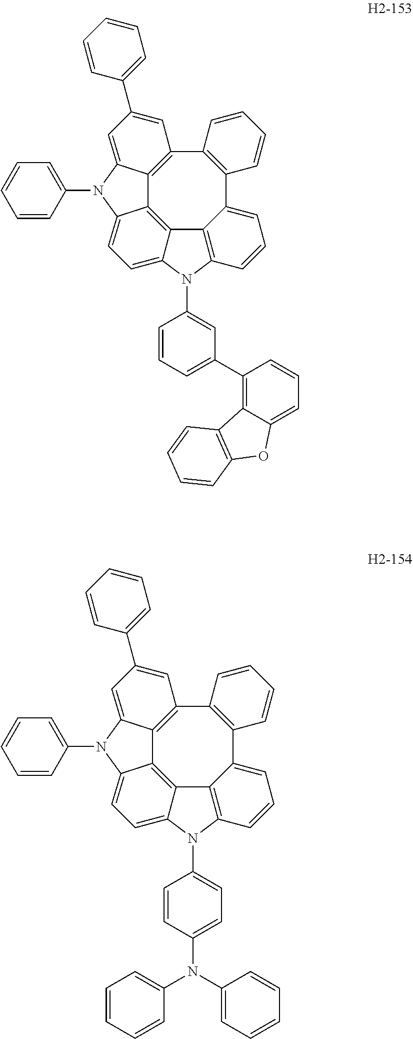 [SMILES: c1ccc(-c2cc3c4ccccc4c4cccc5c4c4c6c3c(c2)n(-c2ccccc2)c6ccc4n5-c2cccc(-c3cccc4oc5ccccc5c34)c2)cc1 ; c1ccc(-c2cc3c4ccccc4c4cccc5c4c4c6c3c(c2)n(-c2ccccc2)c6ccc4n5-c2ccc(N(c3ccccc3)c3ccccc3)cc2)cc1]

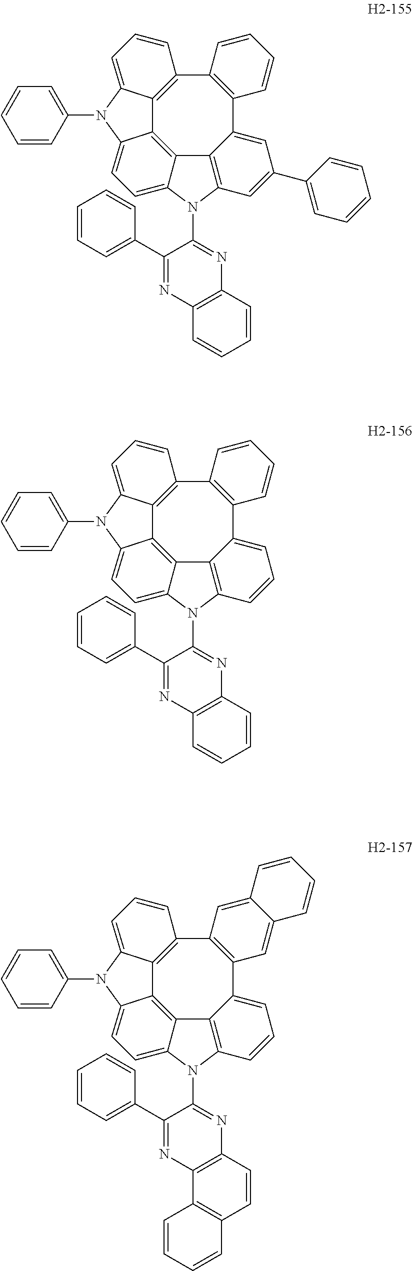 [SMILES: c1ccc(-c2cc3c4ccccc4c4cccc5c4c4c6c3c(c2)n(-c2nc3ccccc3nc2-c2ccccc2)c6ccc4n5-c2ccccc2)cc1 ; c1ccc(-c2nc3ccccc3nc2-n2c3cccc4c5ccccc5c5cccc6c5c5c(c43)c2ccc5n6-c2ccccc2)cc1 ; c1ccc(-c2nc3c(ccc4ccccc43)nc2-n2c3cccc4c5cc6ccccc6cc5c5cccc6c5c5c(c43)c2ccc5n6-c2ccccc2)cc1]

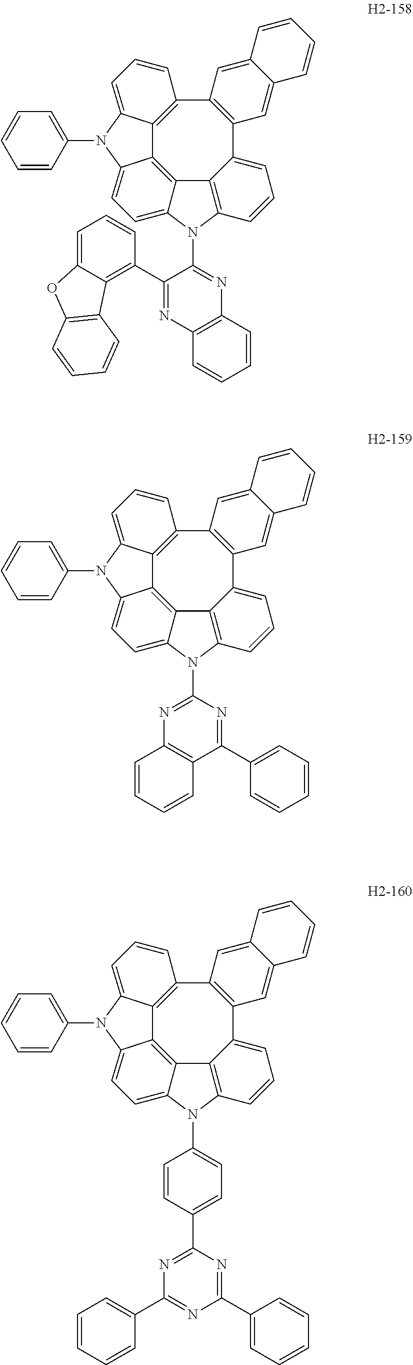 [SMILES: c1ccc(-n2c3cccc4c5cc6ccccc6cc5c5cccc6c5c5c(c43)c2ccc5n6-c2nc3ccccc3nc2-c2cccc3oc4ccccc4c23)cc1 ; c1ccc(-c2nc(-n3c4cccc5c6cc7ccccc7cc6c6cccc7c6c6c(c54)c3ccc6n7-c3ccccc3)nc3ccccc23)cc1 ; c1ccc(-c2nc(-c3ccccc3)nc(-c3ccc(-n4c5cccc6c7cc8ccccc8cc7c7cccc8c7c7c(c65)c4ccc7n8-c4ccccc4)cc3)n2)cc1]

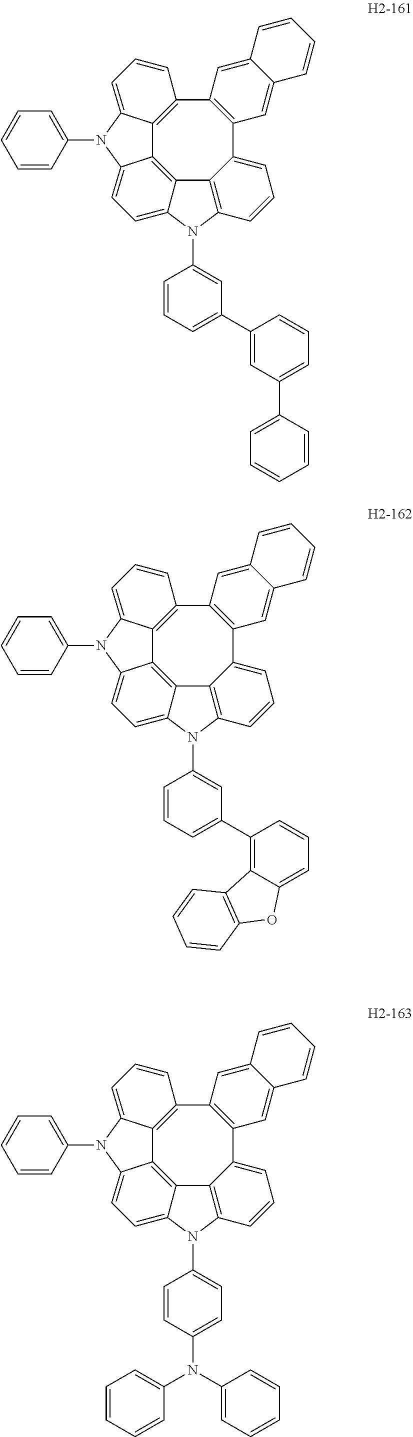

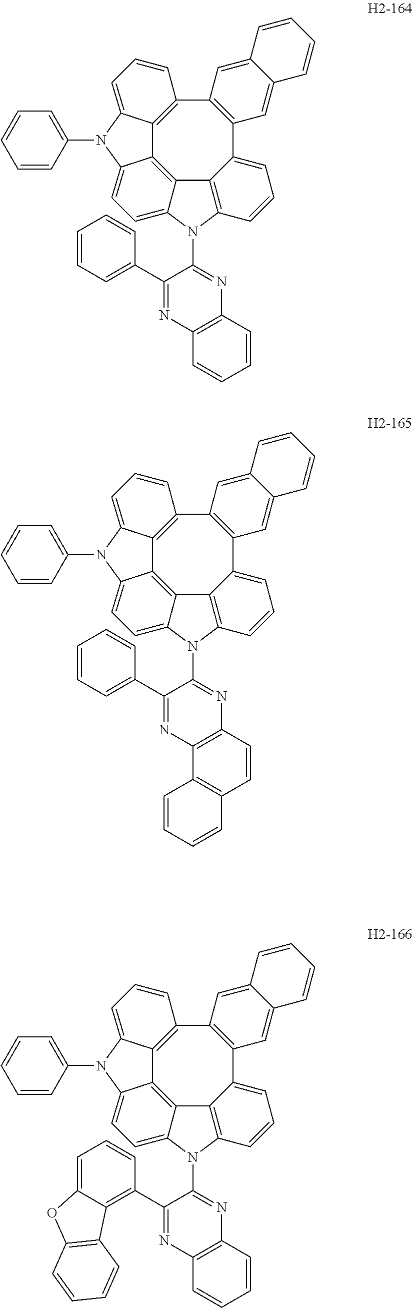 [SMILES: c1ccc(-c2nc3ccccc3nc2-n2c3cccc4c5cc6ccccc6cc5c5cccc6c5c5c(c43)c2ccc5n6-c2ccccc2)cc1 ; c1ccc(-c2nc3c(ccc4ccccc43)nc2-n2c3cccc4c5cc6ccccc6cc5c5cccc6c5c5c(c43)c2ccc5n6-c2ccccc2)cc1 ; c1ccc(-n2c3cccc4c5cc6ccccc6cc5c5cccc6c5c5c(c43)c2ccc5n6-c2nc3ccccc3nc2-c2cccc3oc4ccccc4c23)cc1]

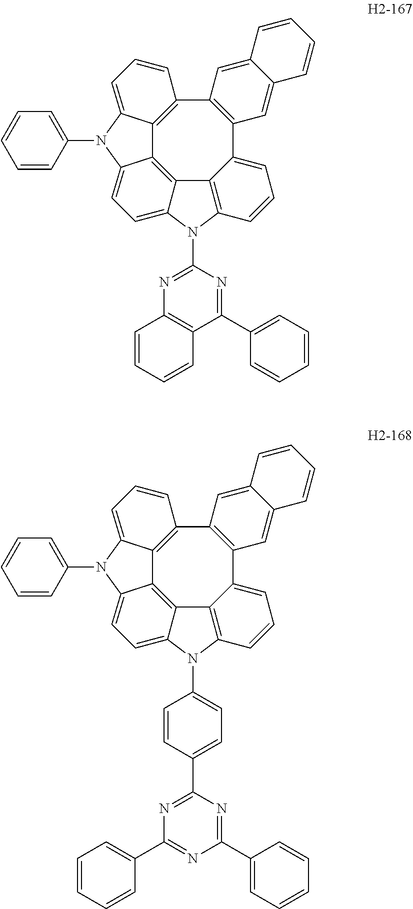 [SMILES: c1ccc(-c2nc(-n3c4cccc5c6cc7ccccc7cc6c6cccc7c6c6c(c54)c3ccc6n7-c3ccccc3)nc3ccccc23)cc1 ; c1ccc(-c2nc(-c3ccccc3)nc(-c3ccc(-n4c5cccc6c7cc8ccccc8cc7c7cccc8c7c7c(c65)c4ccc7n8-c4ccccc4)cc3)n2)cc1]

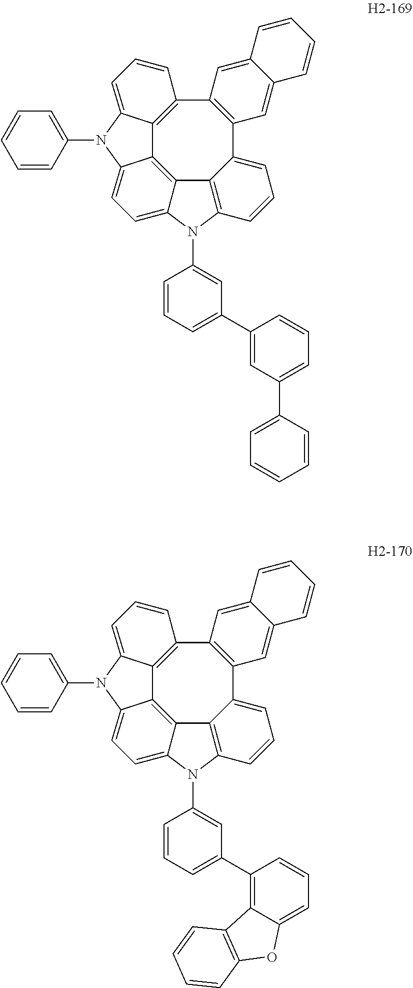 [SMILES: c1ccc(-c2cccc(-c3cccc(-n4c5cccc6c7cc8ccccc8cc7c7cccc8c7c7c(c65)c4ccc7n8-c4ccccc4)c3)c2)cc1 ; c1ccc(-n2c3cccc4c5cc6ccccc6cc5c5cccc6c5c5c(c43)c2ccc5n6-c2cccc(-c3cccc4oc5ccccc5c34)c2)cc1]

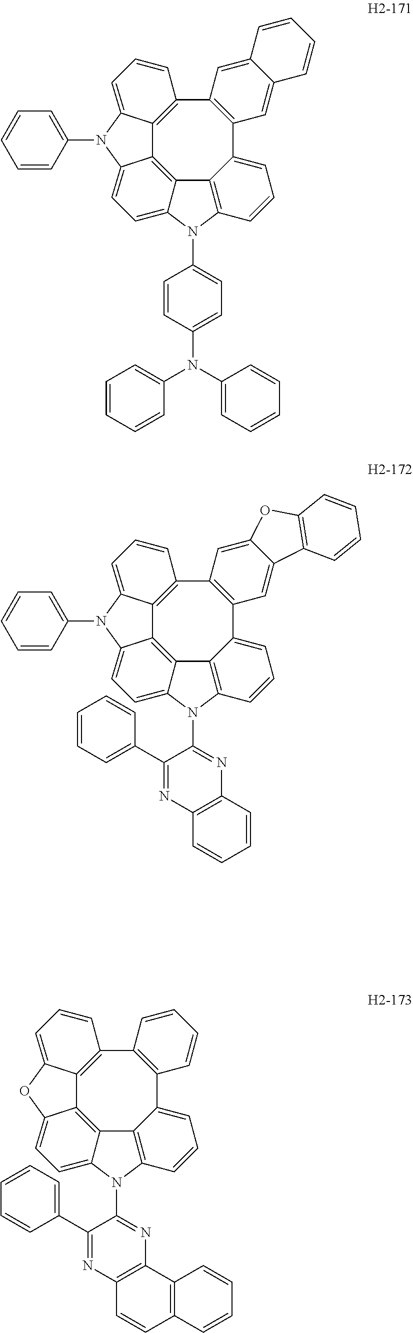 [SMILES: c1ccc(N(c2ccccc2)c2ccc(-n3c4cccc5c6cc7ccccc7cc6c6cccc7c6c6c(c54)c3ccc6n7-c3ccccc3)cc2)cc1 ; c1ccc(-c2nc3ccccc3nc2-n2c3cccc4c5cc6c(cc5c5cccc7c5c5c(c43)c2ccc5n7-c2ccccc2)oc2ccccc26)cc1 ; c1ccc(-c2nc3ccc4ccccc4c3nc2-n2c3cccc4c5ccccc5c5cccc6oc7ccc2c(c7c65)c43)cc1]

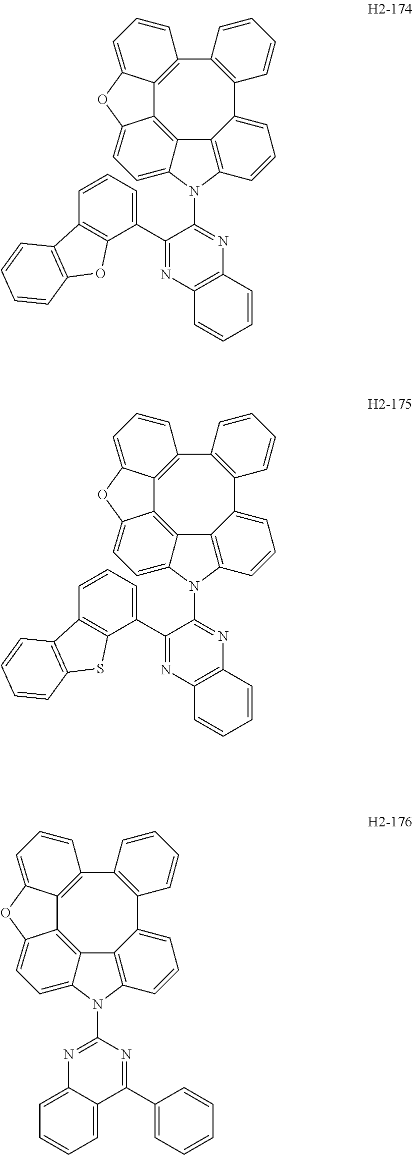 [SMILES: c1ccc2nc(-n3c4cccc5c6ccccc6c6cccc7oc8ccc3c(c8c76)c54)c(-c3cccc4c3oc3ccccc34)nc2c1 ; c1ccc2nc(-n3c4cccc5c6ccccc6c6cccc7oc8ccc3c(c8c76)c54)c(-c3cccc4c3sc3ccccc34)nc2c1 ; c1ccc(-c2nc(-n3c4cccc5c6ccccc6c6cccc7oc8ccc3c(c8c76)c54)nc3ccccc23)cc1]

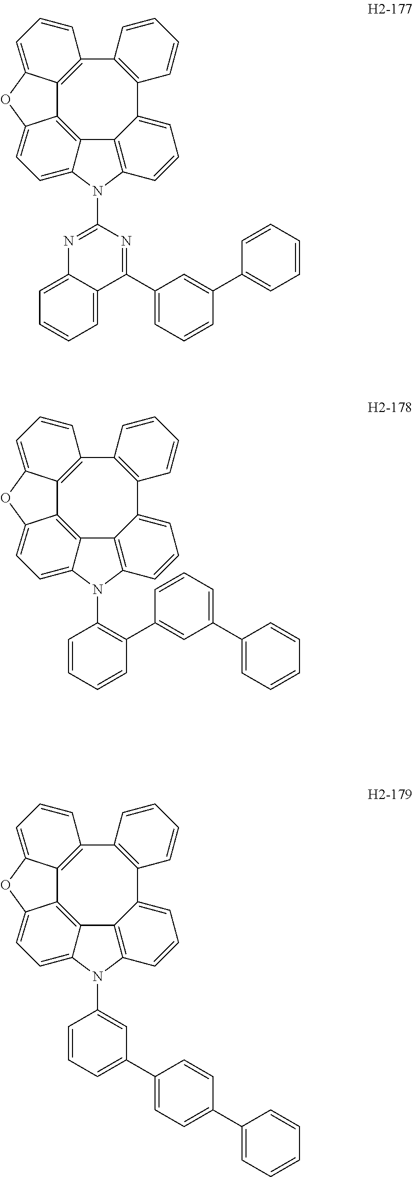 [SMILES: c1ccc(-c2cccc(-c3nc(-n4c5cccc6c7ccccc7c7cccc8oc9ccc4c(c9c87)c65)nc4ccccc34)c2)cc1 ; c1ccc(-c2cccc(-c3ccccc3-n3c4cccc5c6ccccc6c6cccc7oc8ccc3c(c8c76)c54)c2)cc1 ; c1ccc(-c2ccc(-c3cccc(-n4c5cccc6c7ccccc7c7cccc8oc9ccc4c(c9c87)c65)c3)cc2)cc1]

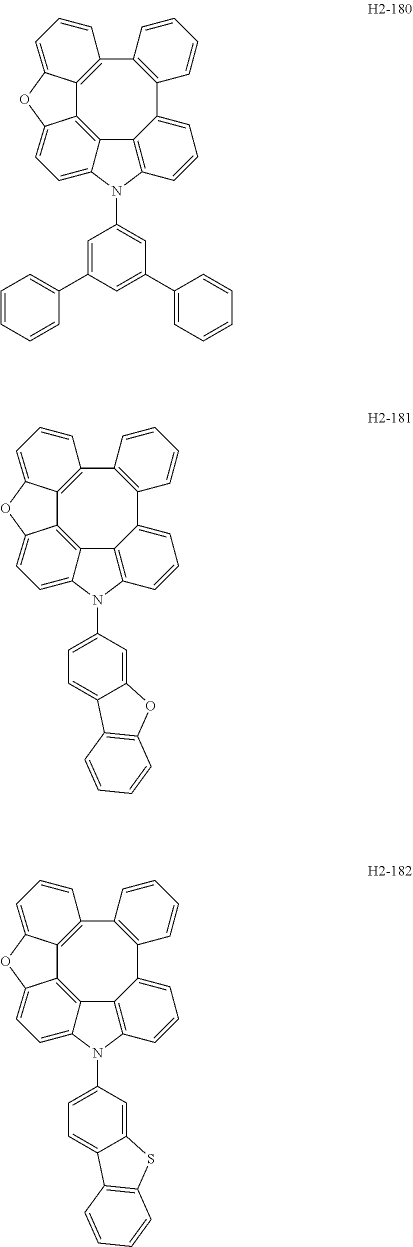 [SMILES: c1ccc(-c2cc(-c3ccccc3)cc(-n3c4cccc5c6ccccc6c6cccc7oc8ccc3c(c8c76)c54)c2)cc1 ; c1ccc2c(c1)oc1cc(-n3c4cccc5c6ccccc6c6cccc7oc8ccc3c(c8c76)c54)ccc12 ; c1ccc2c(c1)sc1cc(-n3c4cccc5c6ccccc6c6cccc7oc8ccc3c(c8c76)c54)ccc12]

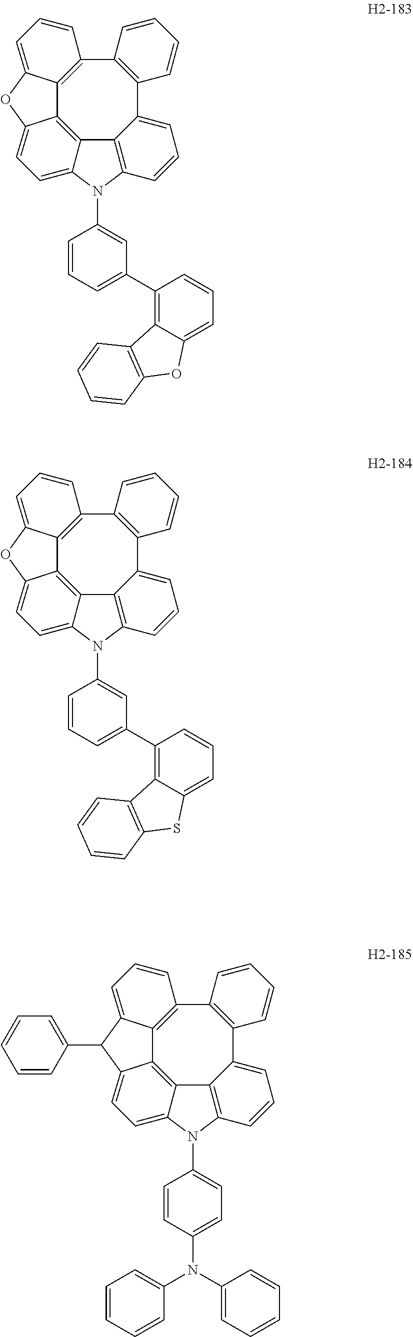 [SMILES: c1cc(-c2cccc3oc4ccccc4c23)cc(-n2c3cccc4c5ccccc5c5cccc6oc7ccc2c(c7c65)c43)c1 ; c1cc(-c2cccc3sc4ccccc4c23)cc(-n2c3cccc4c5ccccc5c5cccc6oc7ccc2c(c7c65)c43)c1 ; c1ccc(C2c3cccc4c5ccccc5c5cccc6c5c5c(c2ccc5n6-c2ccc(N(c5ccccc5)c5ccccc5)cc2)c34)cc1]

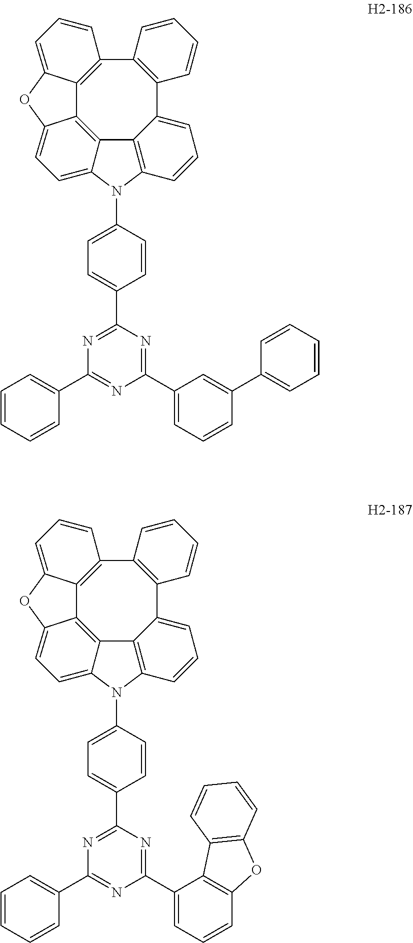 [SMILES: c1ccc(-c2cccc(-c3nc(-c4ccccc4)nc(-c4ccc(-n5c6cccc7c8ccccc8c8cccc9oc%10ccc5c(c%10c98)c76)cc4)n3)c2)cc1 ; c1ccc(-c2nc(-c3ccc(-n4c5cccc6c7ccccc7c7cccc8oc9ccc4c(c9c87)c65)cc3)nc(-c3cccc4oc5ccccc5c34)n2)cc1]

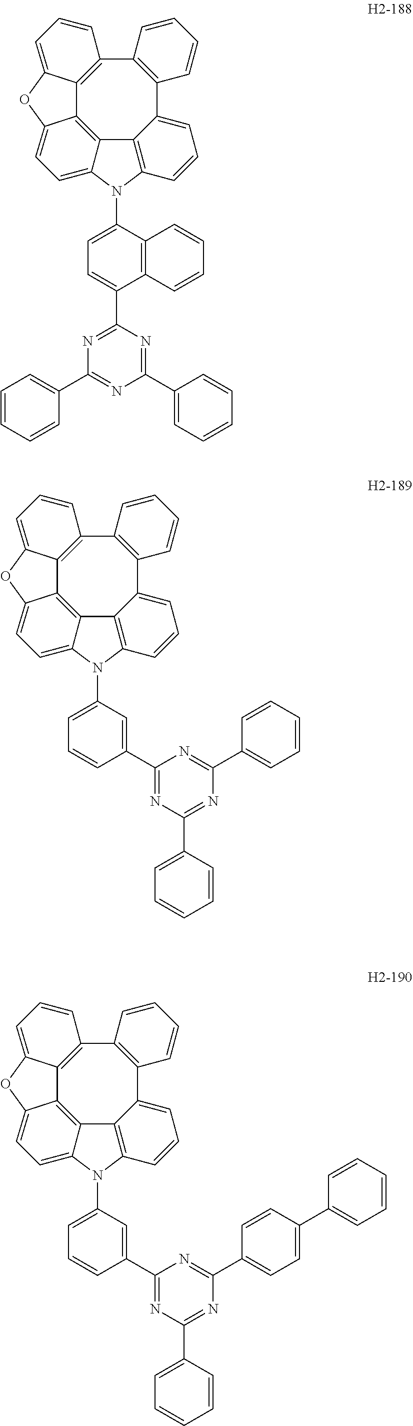 [SMILES: c1ccc(-c2nc(-c3ccccc3)nc(-c3ccc(-n4c5cccc6c7ccccc7c7cccc8oc9ccc4c(c9c87)c65)c4ccccc34)n2)cc1 ; c1ccc(-c2nc(-c3ccccc3)nc(-c3cccc(-n4c5cccc6c7ccccc7c7cccc8oc9ccc4c(c9c87)c65)c3)n2)cc1 ; c1ccc(-c2ccc(-c3nc(-c4ccccc4)nc(-c4cccc(-n5c6cccc7c8ccccc8c8cccc9oc%10ccc5c(c%10c98)c76)c4)n3)cc2)cc1]

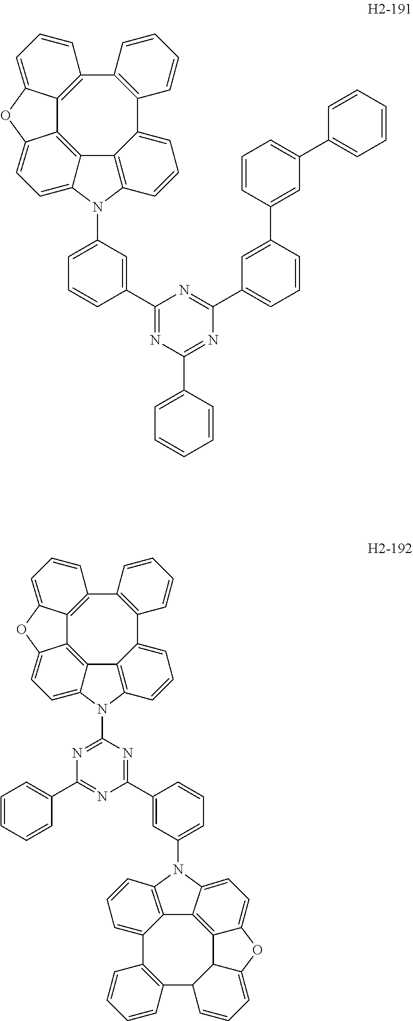 [SMILES: c1ccc(-c2cccc(-c3cccc(-c4nc(-c5ccccc5)nc(-c5cccc(-n6c7cccc8c9ccccc9c9cccc%10oc%11ccc6c(c%11c%109)c87)c5)n4)c3)c2)cc1 ; C1=CC2c3ccccc3-c3cccc4c3c3c5c(ccc3n4-c3cccc(-c4nc(-c6ccccc6)nc(-n6c7cccc8c9ccccc9c9cccc%10oc%11ccc6c(c%11c%109)c87)n4)c3)OC(=C1)C52]

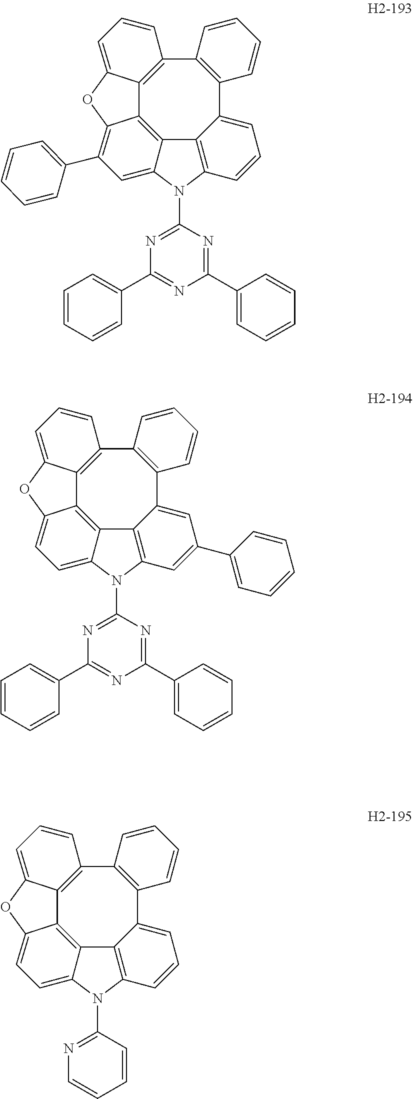 [SMILES: c1ccc(-c2nc(-c3ccccc3)nc(-n3c4cccc5c6ccccc6c6cccc7oc8c(-c9ccccc9)cc3c(c8c76)c54)n2)cc1 ; c1ccc(-c2cc3c4ccccc4c4cccc5oc6ccc7c(c6c54)c3c(c2)n7-c2nc(-c3ccccc3)nc(-c3ccccc3)n2)cc1 ; c1ccc(-n2c3cccc4c5ccccc5c5cccc6oc7ccc2c(c7c65)c43)nc1]

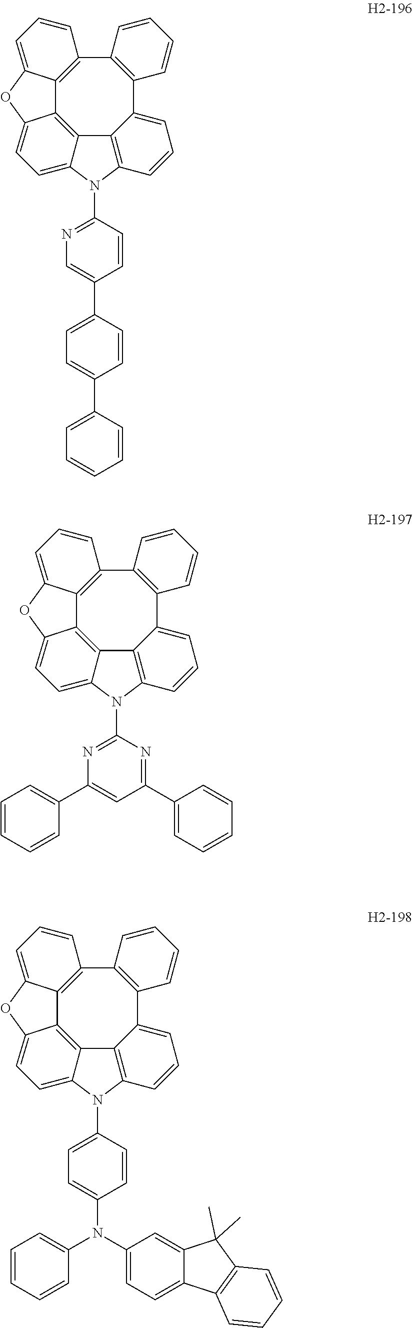 [SMILES: c1ccc(-c2ccc(-c3ccc(-n4c5cccc6c7ccccc7c7cccc8oc9ccc4c(c9c87)c65)nc3)cc2)cc1 ; c1ccc(-c2cc(-c3ccccc3)nc(-n3c4cccc5c6ccccc6c6cccc7oc8ccc3c(c8c76)c54)n2)cc1 ; CC1(C)c2ccccc2-c2ccc(N(c3ccccc3)c3ccc(-n4c5cccc6c7ccccc7c7cccc8oc9ccc4c(c9c87)c65)cc3)cc21]

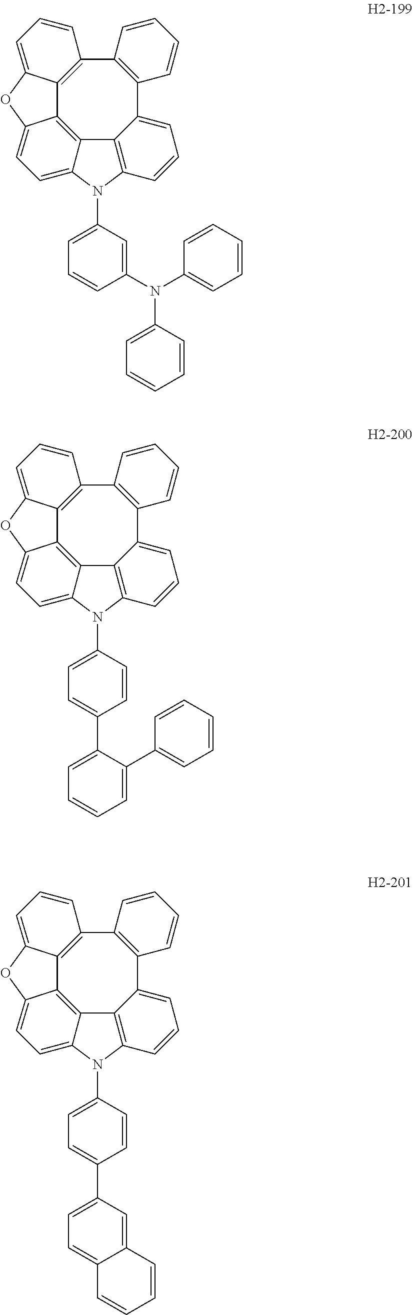 [SMILES: c1ccc(N(c2ccccc2)c2cccc(-n3c4cccc5c6ccccc6c6cccc7oc8ccc3c(c8c76)c54)c2)cc1 ; c1ccc(-c2ccccc2-c2ccc(-n3c4cccc5c6ccccc6c6cccc7oc8ccc3c(c8c76)c54)cc2)cc1 ; c1ccc2cc(-c3ccc(-n4c5cccc6c7ccccc7c7cccc8oc9ccc4c(c9c87)c65)cc3)ccc2c1]

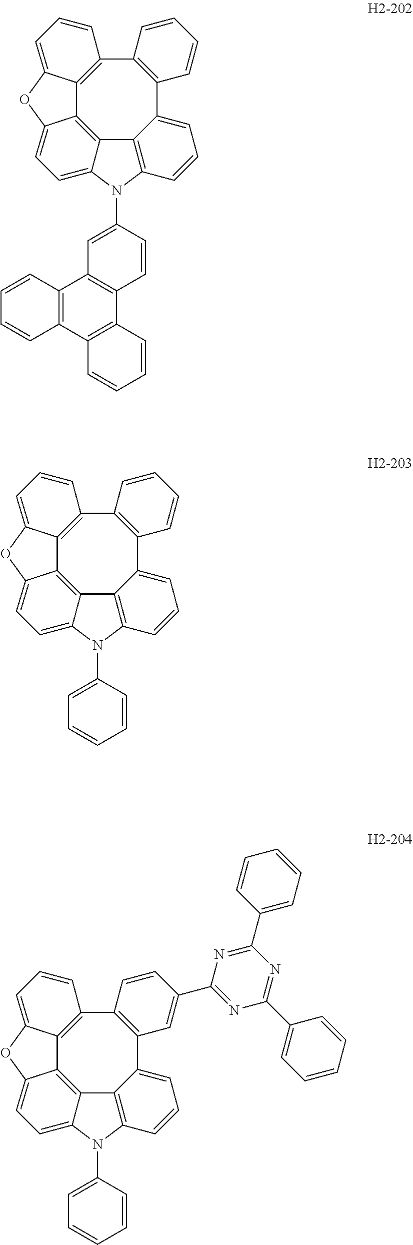 [SMILES: c1ccc2c(c1)c1ccccc1c1cc(-n3c4cccc5c6ccccc6c6cccc7oc8ccc3c(c8c76)c54)ccc21 ; c1ccc(-n2c3cccc4c5ccccc5c5cccc6oc7ccc2c(c7c65)c43)cc1 ; c1ccc(-c2nc(-c3ccccc3)nc(-c3ccc4c(c3)c3cccc5c3c3c6c(ccc3n5-c3ccccc3)oc3cccc4c36)n2)cc1]

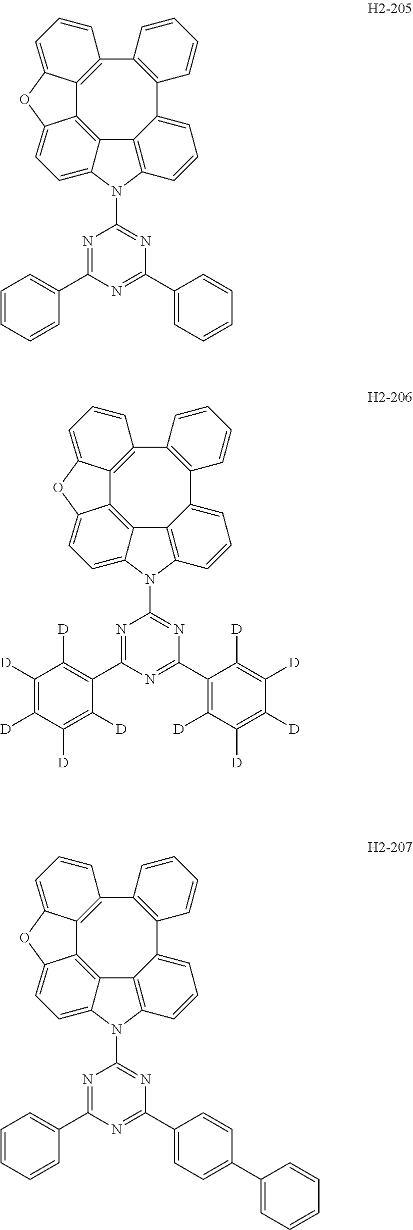 [SMILES: c1ccc(-c2nc(-c3ccccc3)nc(-n3c4cccc5c6ccccc6c6cccc7oc8ccc3c(c8c76)c54)n2)cc1 ; [2H]c1c([2H])c([2H])c(-c2nc(-c3c([2H])c([2H])c([2H])c([2H])c3[2H])nc(-n3c4cccc5c6ccccc6c6cccc7oc8ccc3c(c8c76)c54)n2)c([2H])c1[2H] ; c1ccc(-c2ccc(-c3nc(-c4ccccc4)nc(-n4c5cccc6c7ccccc7c7cccc8oc9ccc4c(c9c87)c65)n3)cc2)cc1]

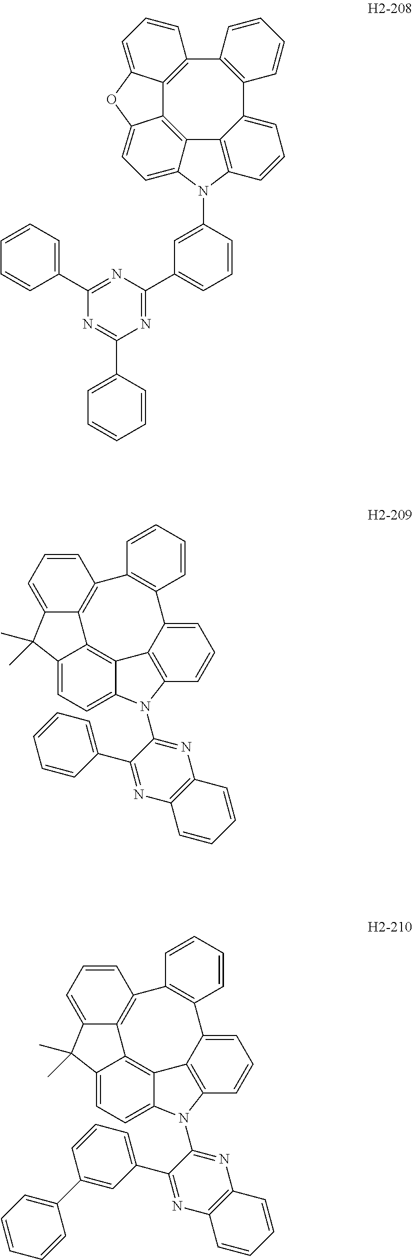 [SMILES: c1ccc(-c2nc(-c3ccccc3)nc(-c3cccc(-n4c5cccc6c7ccccc7c7cccc8oc9ccc4c(c9c87)c65)c3)n2)cc1 ; CC1(C)c2cccc3c4ccccc4c4cccc5c4c4c(c1ccc4n5-c1nc4ccccc4nc1-c1ccccc1)c23 ; CC1(C)c2cccc3c4ccccc4c4cccc5c4c4c(c1ccc4n5-c1nc4ccccc4nc1-c1cccc(-c4ccccc4)c1)c23]

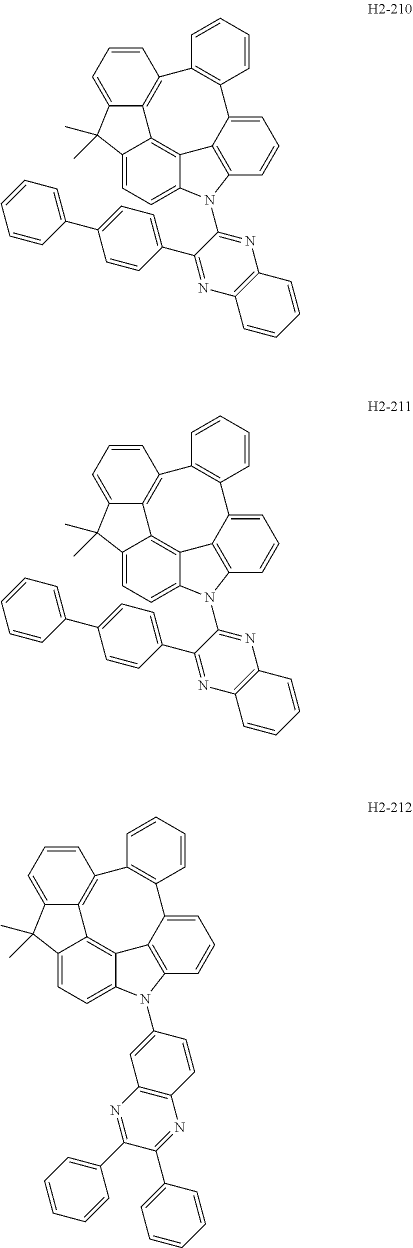 [SMILES: CC1(C)c2cccc3c4ccccc4c4cccc5c4c4c(c1ccc4n5-c1nc4ccccc4nc1-c1ccc(-c4ccccc4)cc1)c23 ; CC1(C)c2cccc3c4ccccc4c4cccc5c4c4c(c1ccc4n5-c1nc4ccccc4nc1-c1ccc(-c4ccccc4)cc1)c23 ; CC1(C)c2cccc3c4ccccc4c4cccc5c4c4c(c1ccc4n5-c1ccc4nc(-c5ccccc5)c(-c5ccccc5)nc4c1)c23]

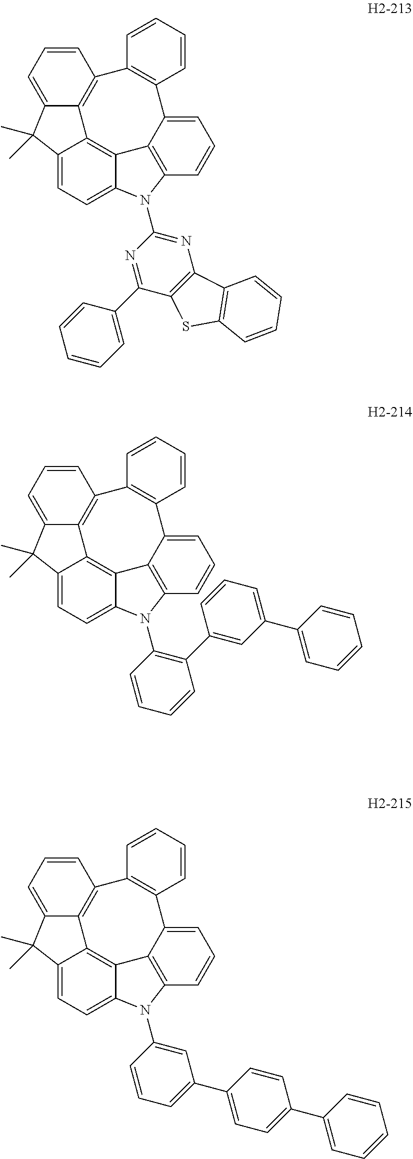 [SMILES: CC1(C)c2cccc3c4ccccc4c4cccc5c4c4c(c1ccc4n5-c1nc(-c4ccccc4)c4sc5ccccc5c4n1)c23 ; CC1(C)c2cccc3c4ccccc4c4cccc5c4c4c(c1ccc4n5-c1ccccc1-c1cccc(-c4ccccc4)c1)c23 ; CC1(C)c2cccc3c4ccccc4c4cccc5c4c4c(c1ccc4n5-c1cccc(-c4ccc(-c5ccccc5)cc4)c1)c23]

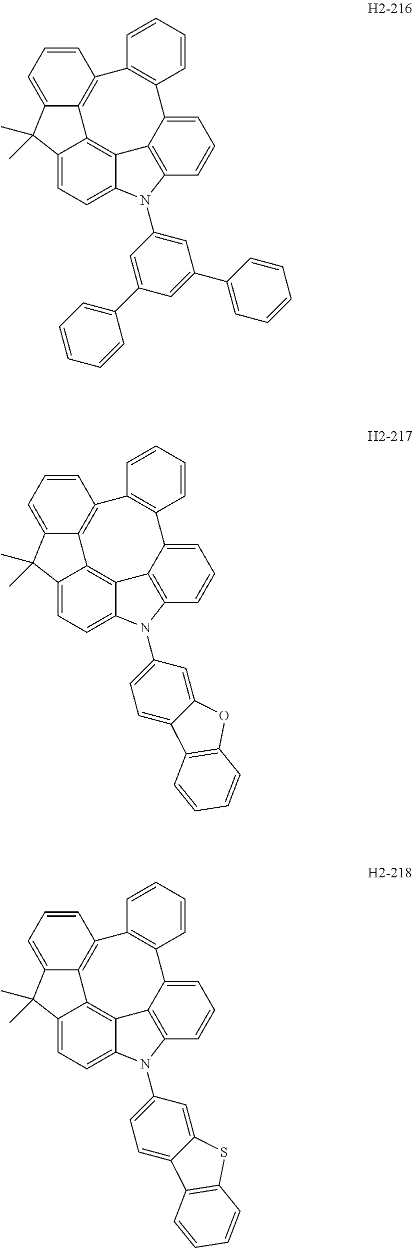 [SMILES: CC1(C)c2cccc3c4ccccc4c4cccc5c4c4c(c1ccc4n5-c1cc(-c4ccccc4)cc(-c4ccccc4)c1)c23 ; CC1(C)c2cccc3c4ccccc4c4cccc5c4c4c(c1ccc4n5-c1ccc4c(c1)oc1ccccc14)c23 ; CC1(C)c2cccc3c4ccccc4c4cccc5c4c4c(c1ccc4n5-c1ccc4c(c1)sc1ccccc14)c23]

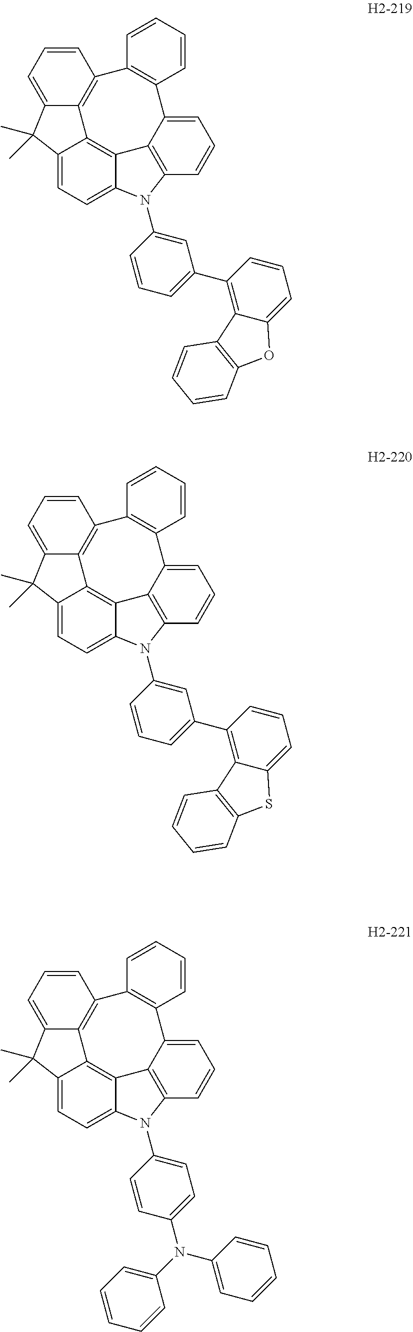 [SMILES: CC1(C)c2cccc3c4ccccc4c4cccc5c4c4c(c1ccc4n5-c1cccc(-c4cccc5oc6ccccc6c45)c1)c23 ; CC1(C)c2cccc3c4ccccc4c4cccc5c4c4c(c1ccc4n5-c1cccc(-c4cccc5sc6ccccc6c45)c1)c23 ; CC1(C)c2cccc3c4ccccc4c4cccc5c4c4c(c1ccc4n5-c1ccc(N(c4ccccc4)c4ccccc4)cc1)c23]

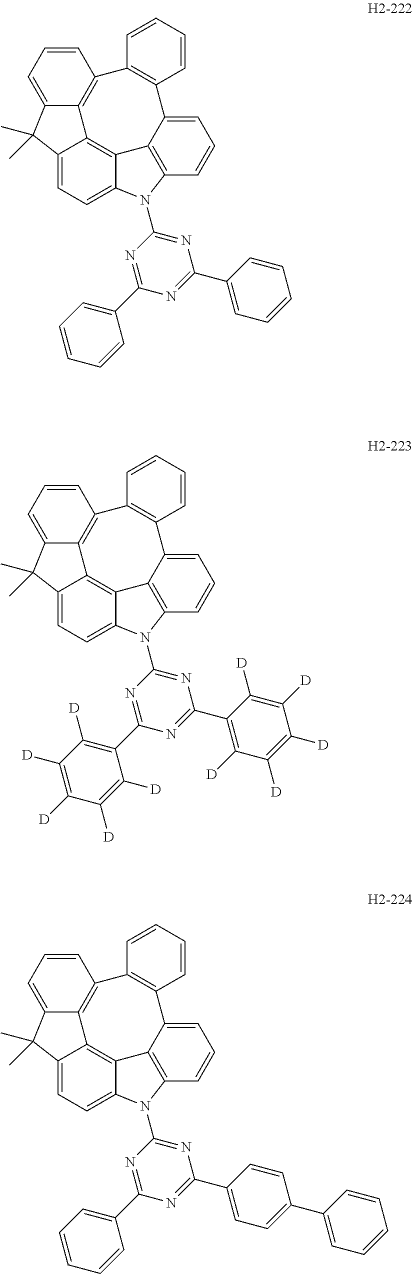 [SMILES: CC1(C)c2cccc3c4ccccc4c4cccc5c4c4c(c1ccc4n5-c1nc(-c4ccccc4)nc(-c4ccccc4)n1)c23 ; [2H]c1c([2H])c([2H])c(-c2nc(-c3c([2H])c([2H])c([2H])c([2H])c3[2H])nc(-n3c4cccc5c6ccccc6c6cccc7c6c6c(ccc3c6c54)C7(C)C)n2)c([2H])c1[2H] ; CC1(C)c2cccc3c4ccccc4c4cccc5c4c4c(c1ccc4n5-c1nc(-c4ccccc4)nc(-c4ccc(-c5ccccc5)cc4)n1)c23]

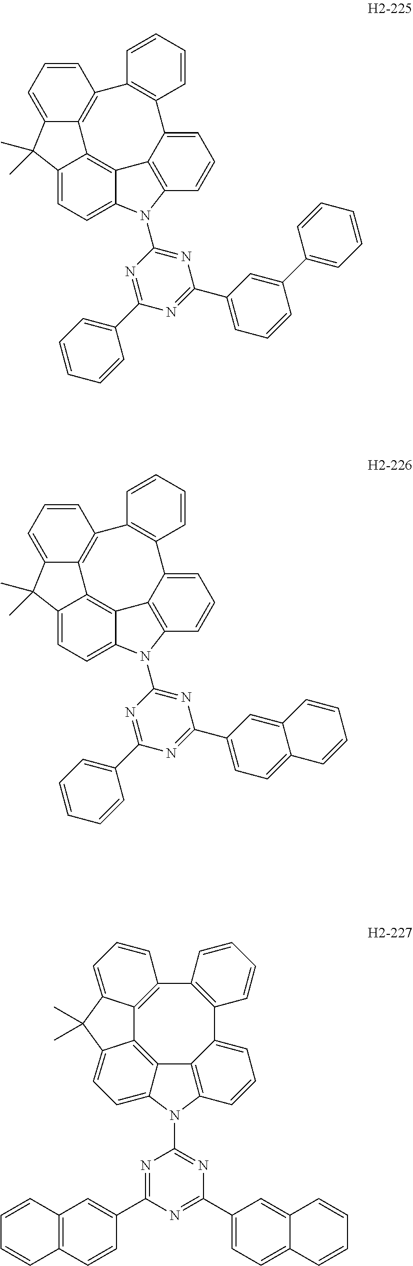 [SMILES: CC1(C)c2cccc3c4ccccc4c4cccc5c4c4c(c1ccc4n5-c1nc(-c4ccccc4)nc(-c4cccc(-c5ccccc5)c4)n1)c23 ; CC1(C)c2cccc3c4ccccc4c4cccc5c4c4c(c1ccc4n5-c1nc(-c4ccccc4)nc(-c4ccc5ccccc5c4)n1)c23 ; CC1(C)c2cccc3c4ccccc4c4cccc5c4c4c(c1ccc4n5-c1nc(-c4ccc5ccccc5c4)nc(-c4ccc5ccccc5c4)n1)c23]

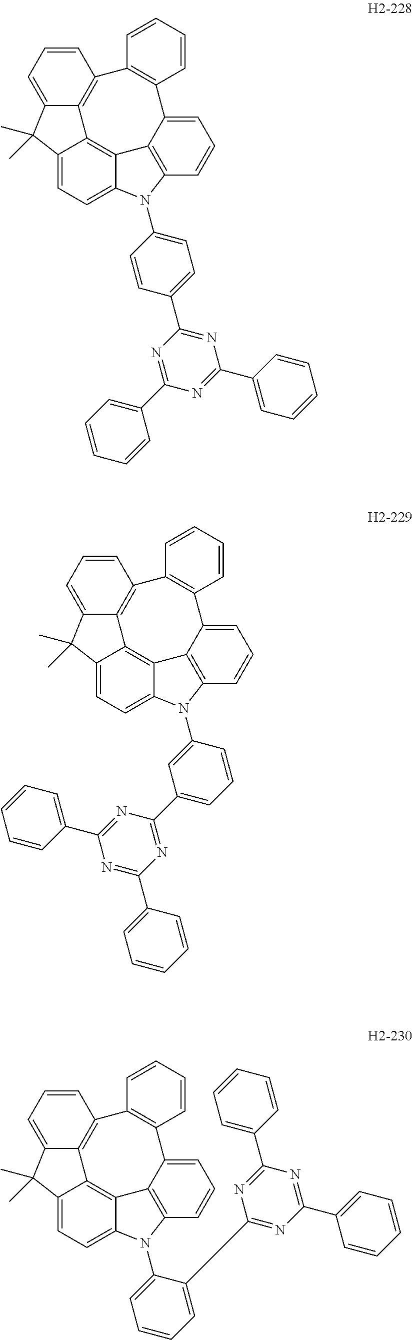 [SMILES: CC1(C)c2cccc3c4ccccc4c4cccc5c4c4c(c1ccc4n5-c1ccc(-c4nc(-c5ccccc5)nc(-c5ccccc5)n4)cc1)c23 ; CC1(C)c2cccc3c4ccccc4c4cccc5c4c4c(c1ccc4n5-c1cccc(-c4nc(-c5ccccc5)nc(-c5ccccc5)n4)c1)c23 ; CC1(C)c2cccc3c4ccccc4c4cccc5c4c4c(c1ccc4n5-c1ccccc1-c1nc(-c4ccccc4)nc(-c4ccccc4)n1)c23]

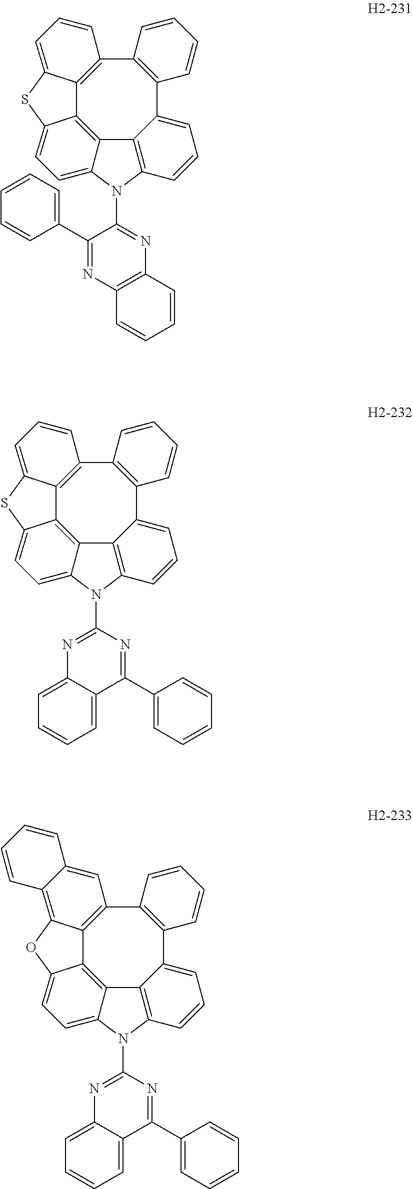 [SMILES: c1ccc(-c2nc3ccccc3nc2-n2c3cccc4c5ccccc5c5cccc6sc7ccc2c(c7c65)c43)cc1 ; c1ccc(-c2nc(-n3c4cccc5c6ccccc6c6cccc7sc8ccc3c(c8c76)c54)nc3ccccc23)cc1 ; c1ccc(-c2nc(-n3c4cccc5c6ccccc6c6cc7ccccc7c7oc8ccc3c(c8c67)c54)nc3ccccc23)cc1]

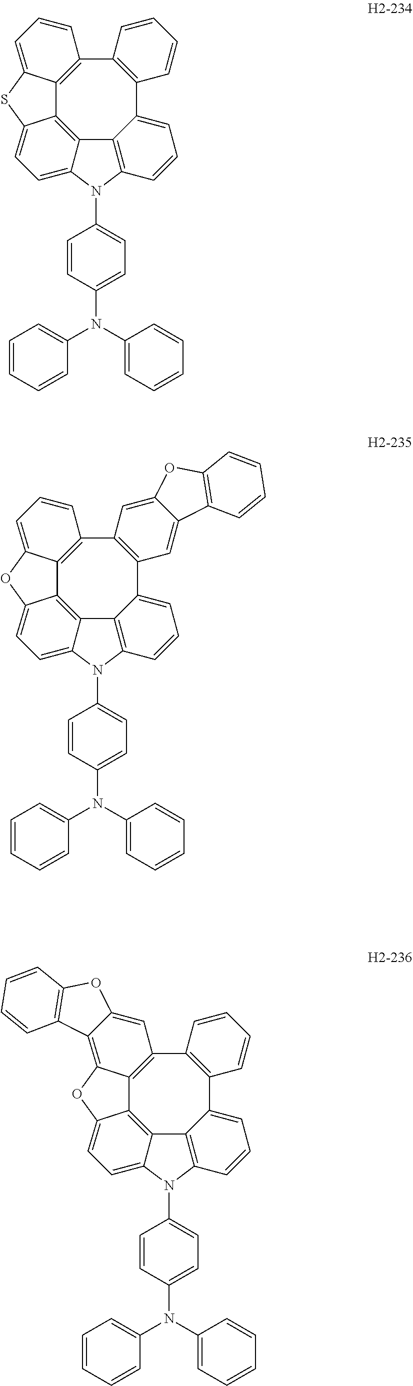 [SMILES: c1ccc(N(c2ccccc2)c2ccc(-n3c4cccc5c6ccccc6c6cccc7sc8ccc3c(c8c76)c54)cc2)cc1 ; c1ccc(N(c2ccccc2)c2ccc(-n3c4cccc5c6cc7c(cc6c6cccc8oc9ccc3c(c9c86)c54)oc3ccccc37)cc2)cc1 ; c1ccc(N(c2ccccc2)c2ccc(-n3c4cccc5c6ccccc6c6cc7oc8ccccc8c7c7oc8ccc3c(c8c67)c54)cc2)cc1]

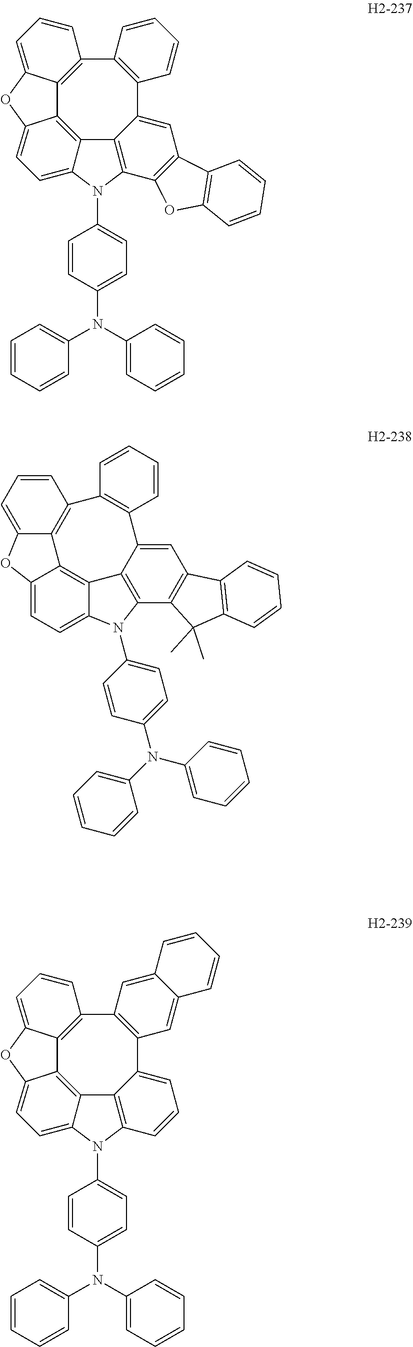 [SMILES: c1ccc(N(c2ccccc2)c2ccc(-n3c4ccc5oc6cccc7c8ccccc8c8cc9c%10ccccc%10oc9c3c8c4c5c67)cc2)cc1 ; CC1(C)c2ccccc2-c2cc3c4ccccc4c4cccc5oc6ccc7c(c6c54)c3c(c21)n7-c1ccc(N(c2ccccc2)c2ccccc2)cc1 ; c1ccc(N(c2ccccc2)c2ccc(-n3c4cccc5c6cc7ccccc7cc6c6cccc7oc8ccc3c(c8c76)c54)cc2)cc1]

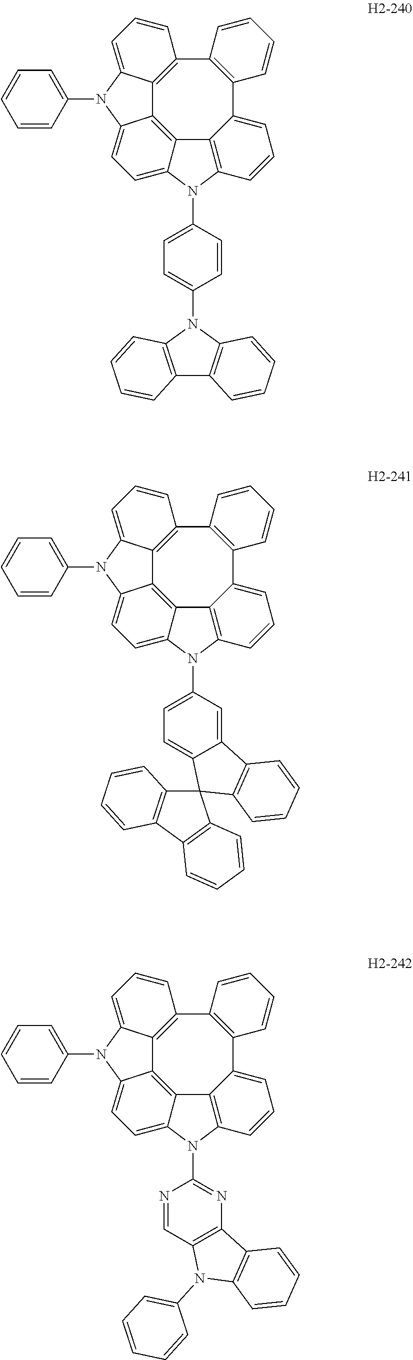 [SMILES: c1ccc(-n2c3cccc4c5ccccc5c5cccc6c5c5c(c43)c2ccc5n6-c2ccc(-n3c4ccccc4c4ccccc43)cc2)cc1 ; c1ccc(-n2c3cccc4c5ccccc5c5cccc6c5c5c(c43)c2ccc5n6-c2ccc3c(c2)-c2ccccc2C32c3ccccc3-c3ccccc32)cc1 ; c1ccc(-n2c3ccccc3c3nc(-n4c5cccc6c7ccccc7c7cccc8c7c7c(c65)c4ccc7n8-c4ccccc4)ncc32)cc1]

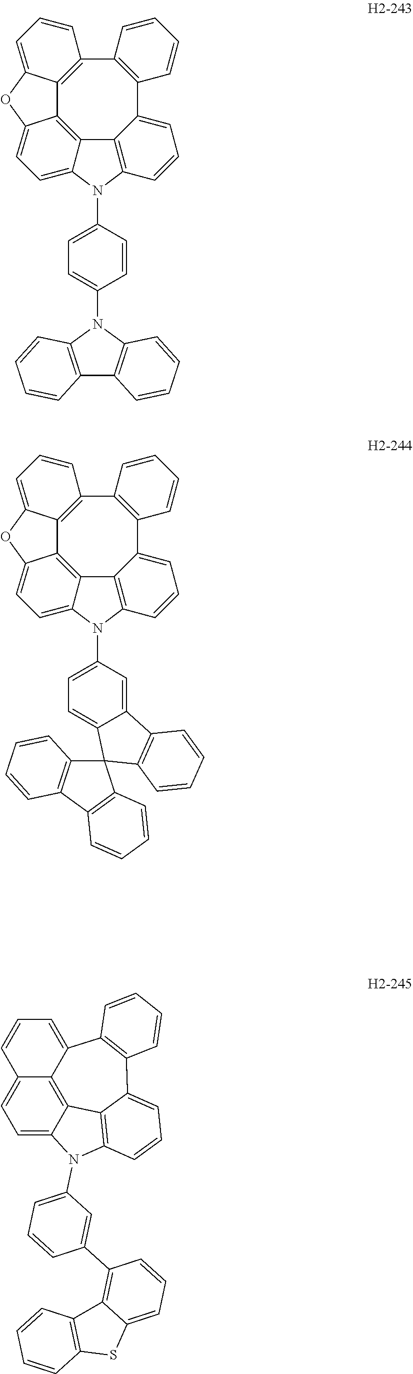 [SMILES: c1ccc2c(c1)c1cccc3oc4ccc5c(c4c31)c1c2cccc1n5-c1ccc(-n2c3ccccc3c3ccccc32)cc1 ; c1ccc2c(c1)-c1ccccc1C21c2ccccc2-c2cc(-n3c4cccc5c6ccccc6c6cccc7oc8ccc3c(c8c76)c54)ccc21 ; c1cc(-c2cccc3sc4ccccc4c23)cc(-n2c3cccc4c3c3c5c(cccc5ccc32)-c2ccccc2-4)c1]

According to one embodiment, the organic electroluminescent compound represented by formula 4 may be more specifically illustrated by the following compounds, but is not limited thereto.

[chemical structure]

[chemical structure]

[chemical structure]

[chemical structure]

[chemical structure]

[chemical structure]

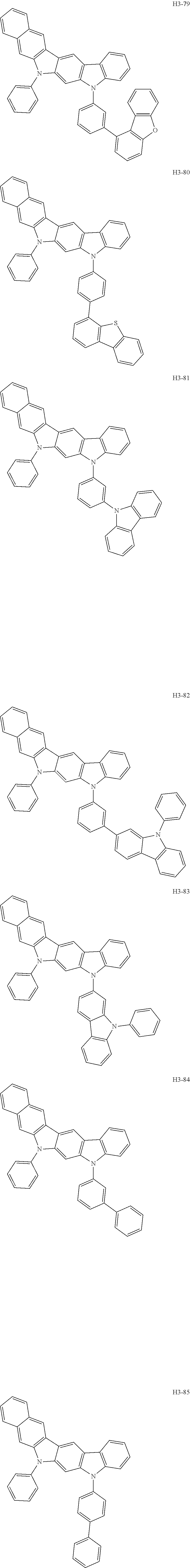

According to one embodiment, the organic electroluminescent compound represented by formula 5 may be more specifically illustrated by the following compounds, but is not limited thereto.

[chemical structure]

[chemical structure]

[chemical structure]

[chemical structure]

[chemical structure]

[chemical structure]

[chemical structure]

[chemical structure]

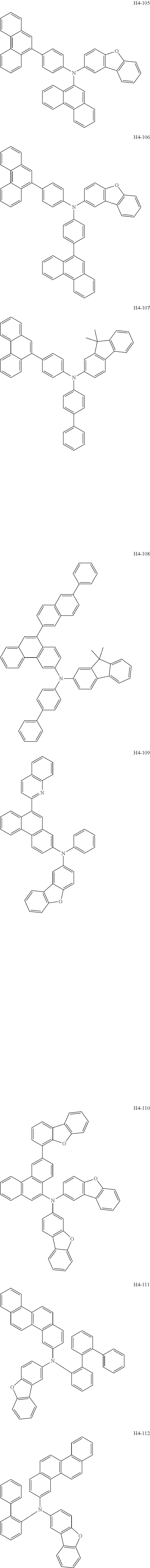

The compounds represented by formulas 2 to 5 according to one embodiment may be prepared with reference to synthetic methods known to those skilled in the art.

Hereinafter, an organic electroluminescent device to which the above-described organic electroluminescent compound and/or the plurality of host materials is(are) applied will be described.

The organic electroluminescent device according to one embodiment includes a first electrode; a second electrode; and at least one organic layer(s) interposed between the first electrode and the second electrode, wherein the organic layer includes at least one light-emitting layer or an electron transport layer. The light-emitting layer or the electron transport layer includes the organic electroluminescent compound represented by formula 1 above.

The organic electroluminescent device according to another embodiment may include a plurality of host materials comprising at least one first host material(s) represented by formula 1 and a second host material which is different from the first host material. The second host material comprises at least one of the organic electroluminescent compound(s) represented by formulas 2 to 5. Wherein the weight ratio of the first host compound to the second host compound may be in the range of about 1:99 to about 99:1, preferably about 10:90 to about 90:10, more preferably about 30:70 to about 70:30, more preferably about 40:60 to about 60:40, and even more preferably about 50:50 in the light-emitting layer. These pluralities of host materials may be included in the same organic layers such as the same light-emitting layer, or may be included in different light-emitting layers, respectively.

According to one embodiment, the organic electroluminescent material of the present disclosure comprises at least one of compound(s) represented by formula 1, i.e., compounds C-1 to C-185, alone or in combination of two or more. These organic electroluminescent materials may be included an organic layer of an organic electroluminescent device, for example, they may be included in the electron transport layer or the light-emitting layer.

According to another embodiment, a plurality of host materials of the present disclosure, for example, may comprise at least one of compound(s) C-1 to C-185 as the first host material and at least one of compound(s) H1-1 to H1-175 as the second host material which is represented by formula 2; may comprise at least one of compound(s) C-1 to C-185 as the first host material and at least one of compound(s) H2-1 to H2-245 as the second host material which is represented by formula 3; may comprise at least one of compound(s) C-1 to C-185 as the first host material and at least one of compound(s) H3-1 to H3-85 as the second host material which is represented by formula 4; or may comprise at least one of compound(s) C-1 to C-185 as the first host material and at least one of compound(s) H4-1 to H4-112 as the second host material which is represented by formula 5.

The organic layer may further comprise at least one layer selected from a hole injection layer, a hole transport layer, a hole auxiliary layer, a light-emitting auxiliary layer, an electron injection layer, an interlayer, a hole blocking layer, an electron blocking layer, and an electron buffer layer in addition to the electron transport layer and the light-emitting layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron blocking material. Also, the electron transport layer, the electron injection layer, the electron buffer layer, or the hole blocking layer may contain the azine-based compound as an electron transport material, an electron injection material, an electron buffer material, or a hole blocking material. Also, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

The plurality of host materials according to one embodiment may be used as light-emitting materials for a white organic light-emitting device. The white organic light-emitting device has suggested various structures such as a parallel side-by-side arrangement method, a stacking arrangement method, or CCM (color conversion material) method, etc., according to the arrangement of R (Red), G (Green), YG (yellowish green), or B (blue) light-emitting units. In addition, the organic electroluminescent material according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. Also, the hole injection layer may be doped as a p-dopant. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may be multi-layers, and wherein each layer may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer may be placed between the electron transport layer (or electron injection layer) and the light-emitting layer, and blocks the arrival of holes to the cathode, thereby improving the probability of recombination of electrons and holes in the light-emitting layer. The hole blocking layer or the electron transport layer may also be multi-layers, wherein each layer may use a plurality of compounds. Also, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron blocking layer. The light-emitting auxiliary layer, the hole auxiliary layer, or the electron blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiO_{X}(1≤X≤2), AlO_{X}(1≤X≤1.5), SiON, SiAlON, etc.; the halogenated metal includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Also, a reductive dopant layer may be employed as a charge generating layer to prepare an organic electroluminescent device having two or more light-emitting layers and emitting white light.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant according to the present disclosure may use the compound represented by the following formula 101, but is not limited thereto.

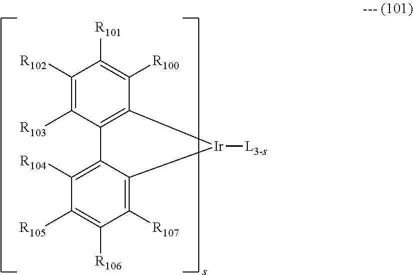

in formula 101,

L is selected from any one of the following structures 1 to 3;

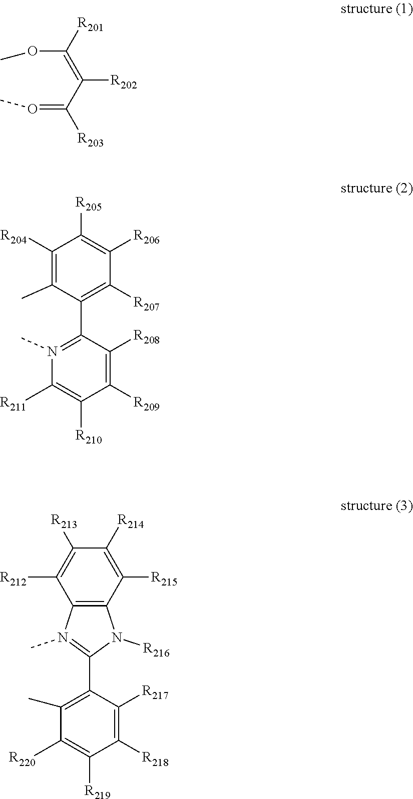

in structures 1 to 3,

R₁₀₀ to R₁₀₃ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; or may be linked to the adjacent substituent to form a substituted or unsubstituted ring(s), e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;

R₁₀₄ to R₁₀₇ each independently represent, hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or may be linked to the adjacent substituent to form a substituted or unsubstituted ring(s), for example, a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;

R₂₀₁ to R₂₂₀ each independently represent, hydrogen, deuterium, halogen, (C1-C30)alkyl unsubstituted or substituted with deuterium and/or halogen, a substituted or unsubstituted (C3-C30)cycloalkyl, or a substituted or unsubstituted (C6-C30)aryl; or may be linked to the adjacent substituent to form a substituted or unsubstituted ring(s); and

s represents an integer of 1 to 3.

Specifically, the specific examples of the dopant compound include the following, but are not limited thereto.

[chemical structure]

[chemical structure]

[chemical structure]

[chemical structure]

[chemical structure]

[chemical structure]

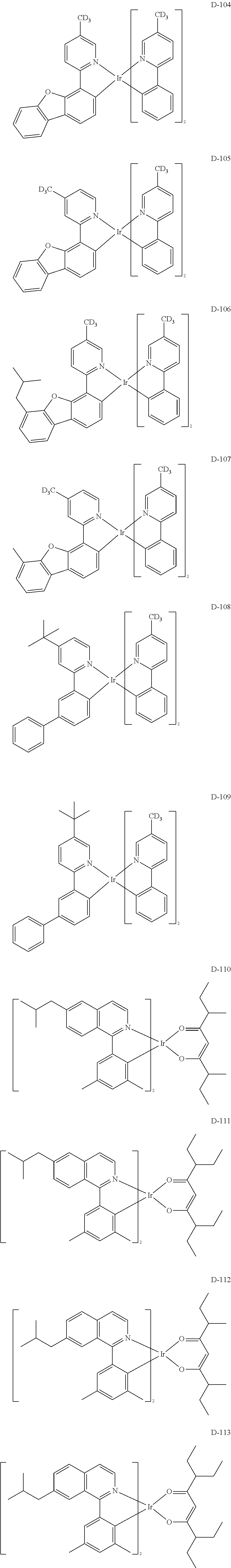

[chemical structure]

[chemical structure]

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by a host compound and a dopant compound according to one embodiment, the layer can be formed by co-deposition or mixed deposition, but is not limited thereto. The co-deposition is a mixed deposition method in which two or more materials are put into respective individual crucible sources and a current is applied to both cells simultaneously to evaporate the materials; and the mixed deposition is a method in which two or more materials are mixed in one crucible source before deposition, and then a current is applied to one cell to evaporate the materials.

According to one embodiment, the present disclosure can provide display devices comprising an organic electroluminescent compound represented by the formula 1, and/or the organic electroluminescent compound represented by formula 1 and any one of the organic electroluminescent compound represented by formulas 2 to 5. In addition, by using the organic electroluminescent device of the present disclosure, display devices such as smartphones, tablets, notebooks, PCs, TVs, or display devices for vehicles, or lighting devices such as outdoor or indoor lighting can be prepared.

Hereinafter, the preparation method of compounds according to the present disclosure will be explained with reference to the synthesis method of a representative compound or intermediate compound in order to understand the present disclosure in detail.

### [Example 1] Synthesis of Compound C-64

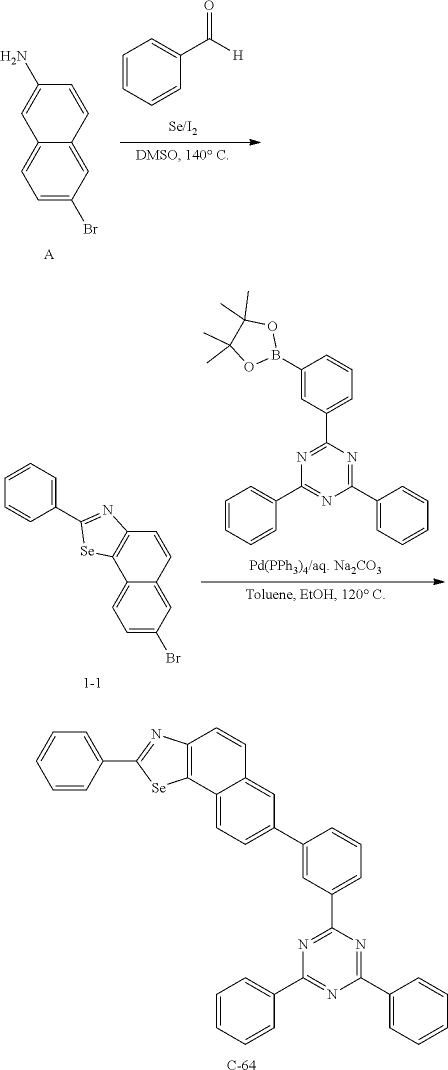

1) Synthesis of Compound 1-1

Compound A (5.0 g, 22.51 mmol), benzaldehyde (2.1 mL, 20.47 mmol), selenium (Se) (1.6 g, 20.47 mmol), iodine (12) (0.5 g, 2.05 mmol), and 100 mL of dimethylsulfoxide (DMSO) were added to the reaction vessel and then stirred at 140° C. for 4 hours. After completion of the reaction, the mixture was cooled to room temperature and then the resulting solid was filtered and washed with ethyl acetate. Next, the filtrate was distilled under reduced pressure followed by being purified by column chromatography to obtain compound 1-1 (5.4 g, yield: 68%).

2) Synthesis of Compound C-64

Compound 1-1 (5.4 g, 13.95 mmol), 2,4-diphenyl-6-(3-(4,4,5,5-tetramethyl-1,3,2-dioxaborolan-2-yl)phenyl)-1,3,5-triazine (6.4 g, 14.65 mmol), tetrakis(triphenylphosphine)palladium (Pd(PPh₃)₄) (0.5 g, 0.42 mmol), sodium carbonate (Na₂CO₃) (3.7 g, 34.88 mmol), 70 mL of toluene, 17 mL of ethanol, and 17 mL of water were added to the reaction vessel and then stirred at 120° C. for 4 hours. After completion of the reaction, the mixture was washed with distilled water and then the organic layers were extracted with ethyl acetate. The extracted organic layer was dried over magnesium sulfate, and then the solvent was removed using a rotary evaporator. Next, it was purified by column chromatography to obtain compound C-64 (5.6 g. yield: 65%).

| | MW | M.P |
|---|---|---|
| C-64 | 615.58 | 293° C. |

### [Example 2] Synthesis of Compound C-89

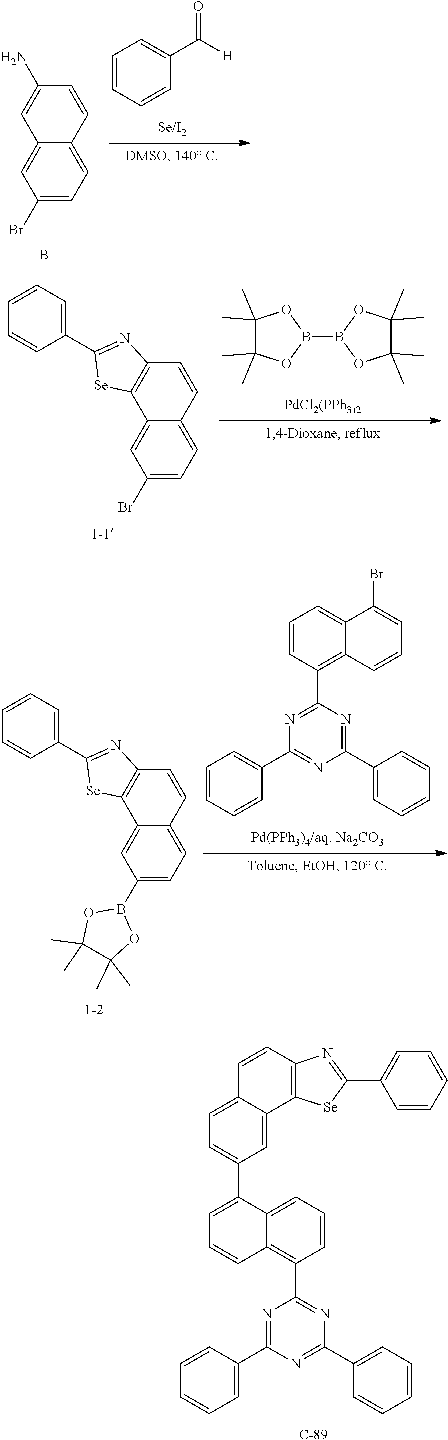

1) Synthesis of Compound 1-1′

Compound B (20.0 g, 90.05 mmol), benzaldehyde (8.4 mL, 81.87 mmol), Se (6.5 g, 81.87 mmol), I₂ (2.1 g, 8.19 mmol), and 400 mL of DMSO were added to the reaction vessel and then stirred at 140° C. for 4 hours. After completion of the reaction, the mixture was cooled to room temperature and then the resulting solid was filtered and washed with ethyl acetate. Next, the filtrate was distilled under reduced pressure followed by being purified by column chromatography to obtain compound 1-1′ (22.7 g, yield: 72%).

2) Synthesis of Compound 1-2

Compound 1-1′ (15.0 g, 38.75 mmol), bis(pinacolato)diborane (11.8 g, 46.50 mmol), bis(triphenylphosphine)palladium(II)dichloride (PdCl₂(PPh₃)₂) (2.7 g, 3.88 mmol), potassium acetate (KOAc) (9.5 g, 96.88 mmol) and 200 mL of 1,4-dioxane were added to the reaction vessel and then stirred under reflux for 6 hours. After completion of the reaction, the mixture was cooled to room temperature and then the organic layers were extracted with ethyl acetate. The extracted organic layer was dried over magnesium sulfate, and then the solvent was removed using a rotary evaporator. Next, it was purified by column chromatography to obtain compound 1-2 (14.8 g, yield: 88%).

3) Synthesis of Compound C-89

Compound 1-2 (5.1 g, 11.75 mmol), 2-(5-bromonaphthalen-1-yl)-4,6-diphenyl-1,3,5-triazine (5.1 g, 11.75 mmol), Pd(PPh₃)₄ (0.4 g, 0.35 mmol), Na₂CO₃ (3.1 g, 29.38 mmol), 60 mL of toluene, 15 mL of ethanol, and 5 mL of water were added to the reaction vessel and then stirred at 120° C. for 3 hours. After completion of the reaction, the mixture was washed with distilled water and then the organic layers were extracted with ethyl acetate. The extracted organic layer was dried over magnesium sulfate, and then the solvent was removed using a rotary evaporator. Next, it was purified by column chromatography to obtain compound C-89 (3.4 g, yield: 44%).

| | MW | M.P |
|---|---|---|
| C-89 | 665.34 | 286° C. |

### [Example 3] Synthesis of Compound C-111

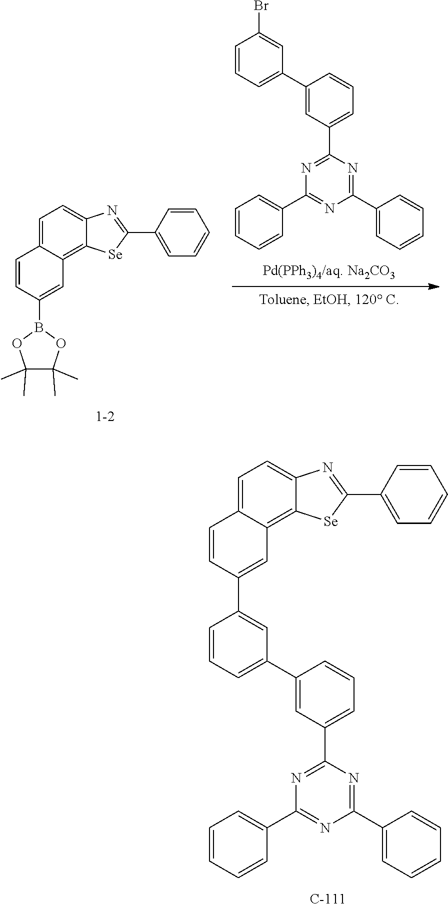

Compound 1-2 (5.4 g, 12.44 mmol), 2-(3′-bromo-[1,1′-biphenyl]-3-yl)-4,6-diphenyl)-1,3,5-triazine (5.8 g, 12.44 mmol), Pd(PPh₃)₄ (0.4 g, 0.37 mmol), Na₂CO₃ (3.3 g, 31.10 mmol), 62 mL of toluene, 15 mL of ethanol, and 15 mL of water were added to the reaction vessel and then stirred at 120° C. for 4 hours. After completion of the reaction, the mixture was washed with distilled water and then the organic layers were extracted with ethyl acetate. The extracted organic layer was dried over magnesium sulfate, and then the solvent was removed using a rotary evaporator. Next, it was purified by column chromatography to obtain compound C-111 (3.5 g, yield: 41%).

| | MW | M.P |
|---|---|---|
| C-111 | 691.68 | 275° C. |

### [Example 4] Synthesis of Compound C-95

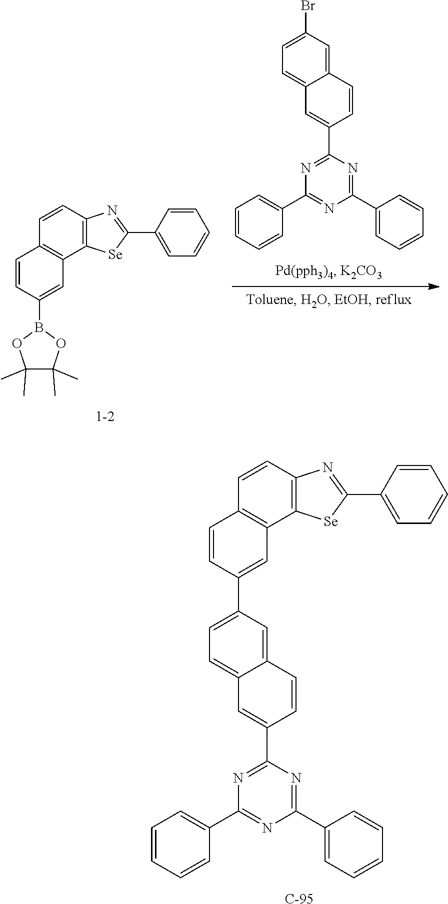

Compound 1-2 (5 g, 11.52 mmol), 2-(6-bromonaphthalene-2-yl)-4,6-diphenyl-1,3,5-triazine (5.6 g, 12.66 mmol), Pd(PPh₃)₄ (0.40 g, 0.35 mmol), potassium carbonate (K₂CO₃) (4.0 g, 28.80 mmol), 60 mL of toluene, 15 mL of ethanol, and 15 mL of water were added to the flask and then dissolved, followed by stirring under reflux at 120° C. for 4 hours. After completion of the reaction, the organic layers were extracted with ethyl acetate. The extracted organic layer was dried over magnesium sulfate. Next, it was distilled under reduced pressure and separated by column chromatography to obtain compound C-95 (2.0 g, yield: 24%).

| | MW | M.P |
|---|---|---|
| C-95 | 666.64 | 288.0° C. |

### [Example 5] Synthesis of Compound C-105

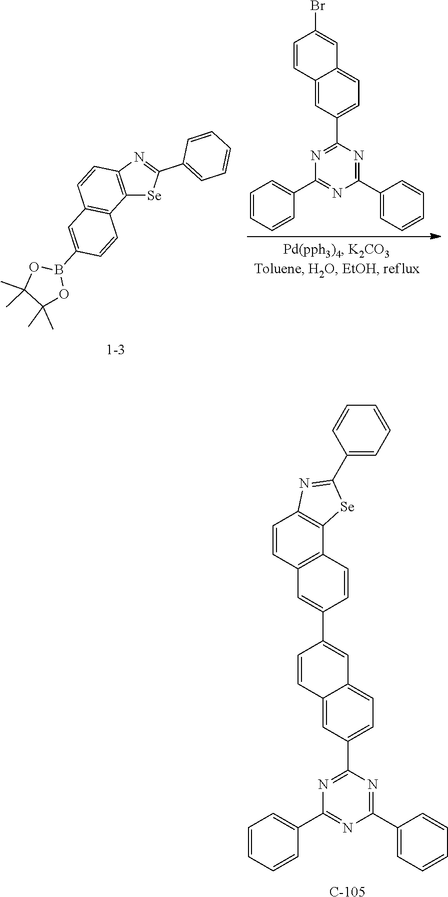

Compound 1-3 (6 g, 13.82 mmol), 2-(6-bromonaphthalene-2-yl)-4,6-diphenyl-1,3,5-triazine (6.7 g, 15.20 mmol), Pd(PPh₃)₄ (0.48 g, 0.42 mmol), K₂CO₃ (4.8 g, 34.55 mmol), 70 mL of toluene, 18 mL of ethanol, and 18 mL of water were added to the flask and then dissolved, followed by stirring under reflux at 120° C. for 4 hours. After completion of the reaction, the organic layers were extracted with ethyl acetate and then dried over magnesium sulfate. Next, it was distilled under reduced pressure and separated by column chromatography to obtain compound C-105 (2.1 g, yield: 23%).

| | MW | M.P |
|---|---|---|
| C-105 | 666.64 | 339.0° C. |

### [Example 6] Synthesis of Compound C-104

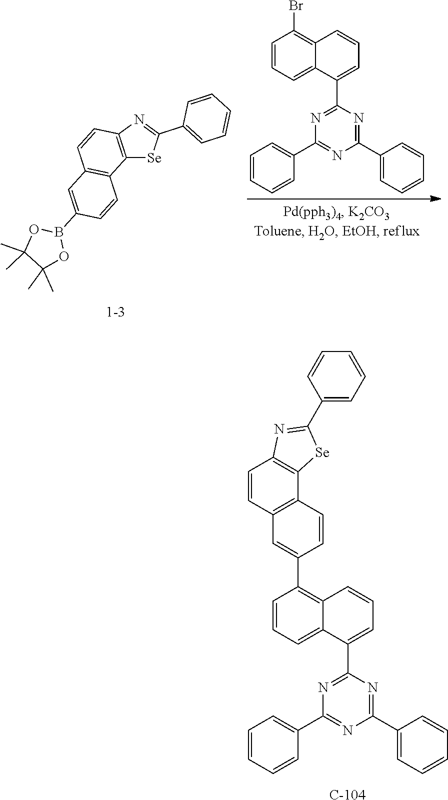

Compound 1-3 (6 g, 13.82 mmol), 2-(5-bromonaphthalene-1-yl)-4,6-diphenyl-1,3,5-triazine (6.7 g, 15.20 mmol), Pd(PPh₃)₄ (0.48 g, 0.42 mmol), K₂CO₃ (4.8 g, 34.55 mmol), 70 mL of toluene, 18 mL of ethanol, and 18 mL of water were added to the flask and then dissolved, followed by stirring under reflux at 120° C. for 4 hours. After completion of the reaction, the organic layers were extracted with ethyl acetate and then dried over magnesium sulfate. Next, it was distilled under reduced pressure and separated by column chromatography to obtain compound C-104 (3.2 g, yield: 34%).

| | MW | M.P |
|---|---|---|
| C-104 | 666.64 | 323.0° C. |

### [Example 7] Synthesis of Compound C-174

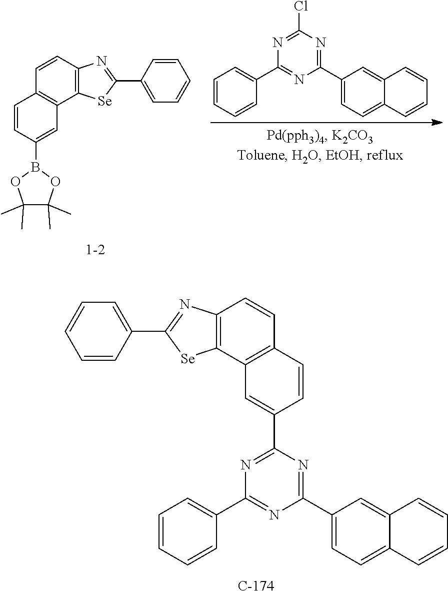

Compound 1-2 (4.0 g, 9.2 mmol), 2-chloro-4-(naphthalene-2-yl)-6-phenyl-1,3,5-triazine (3.5 g, 11.0 mmol), Pd(PPh₃)₄ (0.53 g, 0.5 mmol), K₂CO₃ (2.5 g, 18.4 mmol), toluene (36 mL), ethanol (7 mL), and distilled water (9 mL) were added to the reaction vessel and then stirred at 130° C. for 6 hours. After completion of the reaction, the mixture was added dropwise to methanol, and the resulting solid was filtered. Next, the resulting solid was purified by column chromatography to obtain compound C-174 (2.2 g, yield: 41%).

| | MW | M.P |
|---|---|---|
| C-174 | 589.6 | 189° C. |

### [Example 8] Synthesis of Compound C-175

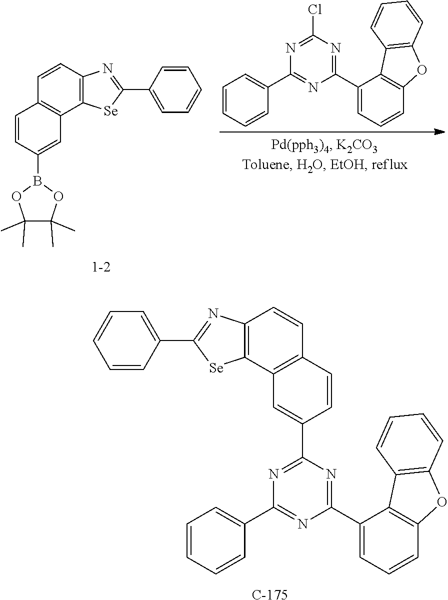

Compound 1-2 (4.0 g, 9.2 mmol), 2-chloro-4-(dibenzo[b,d]furan-1-yl)-6-phenyl-1,3,5-triazine (4.0 g, 11.0 mmol), Pd(PPh₃)₄ (0.53 g, 0.5 mmol), K₂CO₃ (2.5 g, 18.4 mmol), toluene (36 mL), ethanol (7 mL), and distilled water (9 mL) were added to the reaction vessel and then stirred at 130° C. for 6 hours. After completion of the reaction the mixture was added dropwise to methanol, and the resulting solid was filtered. Next, the resulting solid was rifled b column chromatography to obtain compound C-175 (4.8 g, yield: 83%).

| | MW | M.P |
|---|---|---|
| C-175 | 629.6 | 312° C. |

### [Example 9] Synthesis of Compound C-184

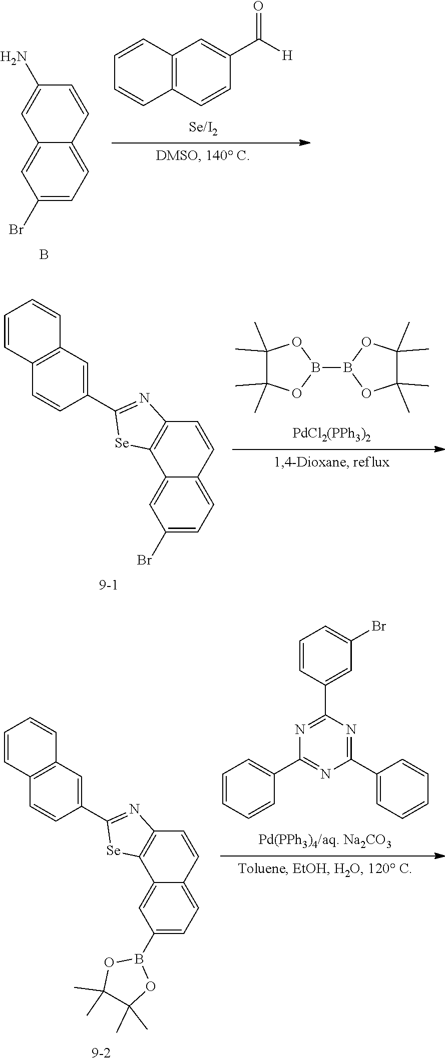

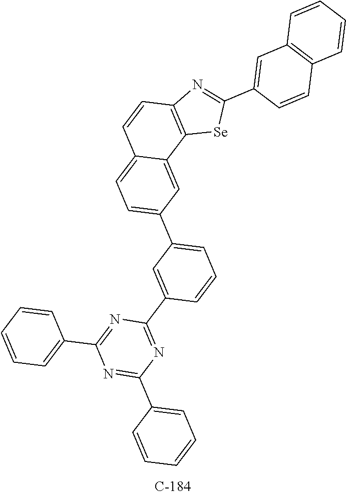 [SMILES: c1ccc(-c2nc(-c3ccccc3)nc(-c3cccc(-c4ccc5ccc6nc(-c7ccc8ccccc8c7)[se]c6c5c4)c3)n2)cc1]

1) Synthesis of Compound 9-1

Compound B (18.0 g, 81.05 mmol), 2-naphthaldehyde (11.51 g, 73.68 mmol), Se (5.82 g, 73.68 mmol), I₂ (1.87 g, 7.37 mmol), and 370 mL of DMSO were added to the reaction vessel, and then stirred at 140° C. for 4 hours. After completion of the reaction, the mixture was cooled to room temperature and then the resulting solid was filtered and washed with ethyl acetate. Next, the filtrate was distilled under reduced pressure followed by being purified by column chromatography to obtain compound 9-1 (13 g, yield: 36%).

2) Synthesis of Compound 9-2

Compound 9-1 (13.0 g, 29.73 mmol), bis(pinacolato)diborane (9.1 g, 35.68 mmol), PdCl₂(PPh₃)₂ (2.1 g, 2.97 mmol), KOAc (5.8 g, 59.47 mmol) and 150 mL of 1,4-dioxane were added to the reaction vessel and then stirred under reflux for 4 hours. After completion of the reaction, the mixture was cooled to room temperature and then the organic layers were extracted with ethyl acetate. The extracted organic layer was dried over magnesium sulfate, and then the solvent was removed using a rotary evaporator. Next, it was purified by column chromatography to obtain compound 9-2 (11.0 g, yield: 78%).

3) Synthesis of Compound C-184

Compound 9-2 (5.0 g, 10.33 mmol), 2-(3-bromophenyl)-4,6-diphenyl-1,3,5-triazine (4.4 g, 11.36 mmol), Pd(PPh₃)₄ (0.36 g, 0.31 mmol), K₂CO₃ (3.6 g, 25.8 mmol), 51 mL of toluene, 13 mL of ethanol, and 13 mL of water were added to the reaction vessel and then stirred at 120° C. for 4 hours. After completion of the reaction, the mixture was washed with distilled water and then the organic layers were extracted with ethyl acetate. The extracted organic layer was dried over magnesium sulfate, and then the solvent was removed using a rotary evaporator. Next, it was purified by column chromatography to obtain compound C-184 (4.9 g, yield: 71%).

| | MW | M.P |
|---|---|---|
| C-184 | 666.64 | 348° C. |

### [Example 10] Synthesis of Compound C-94

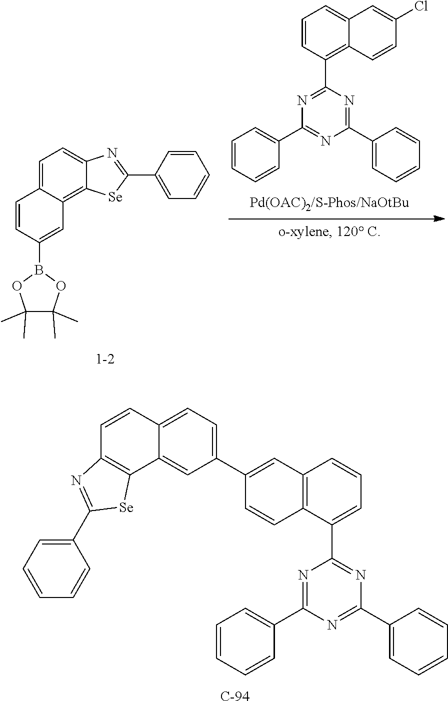

Compound 1-2 (2.5 g, 6.33 mmol), Compound 2-(6-chloronaphthalene-1-yl)-4,6-diphenyl-1,3,5-triazine (3.3 g, 7.60 mmol), Pd(OAC)₂ (0.04 g, 0.19 mmol), S-Phos (0.15 g, 0.38 mmol), and NaOtBu (0.9 g, 9.50 mmol) in the reaction vessel were dissolved in 63 mL of o-xylene, and then the mixture was stirred under reflux for 5 hours. After completion of the reaction, the mixture was cooled to room temperature and then, filtered, separated with a silica filter to form a solid, followed by recrystallized to obtain compound C-94 (2.8 g, yield: 66.6%).

| | MW | M.P |
|---|---|---|
| C-94 | 665.65 | 301° C. |

### [Example 11] Synthesis of Compound C-183

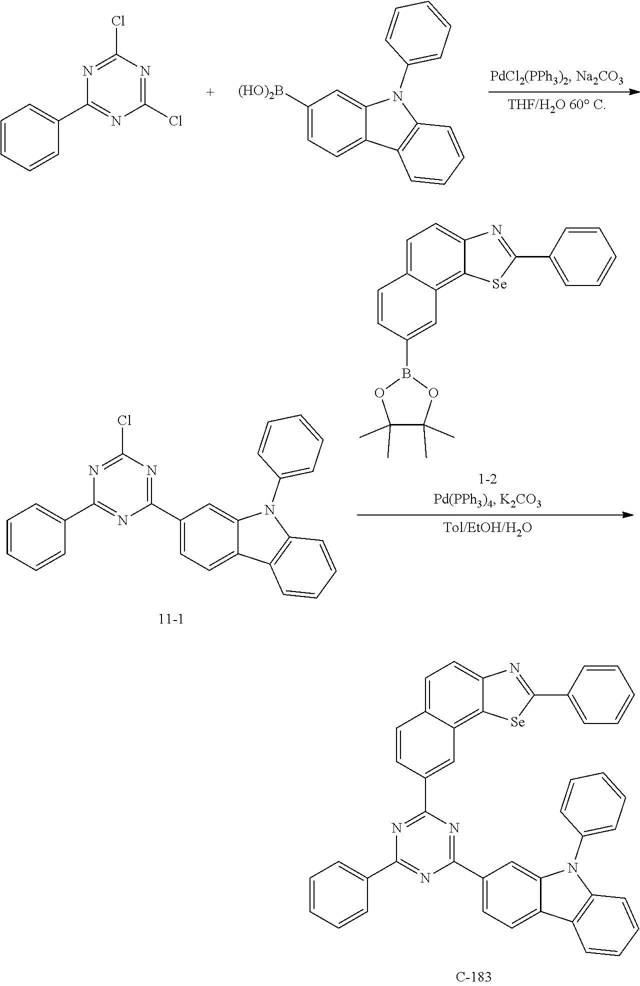

1) Synthesis of Compound 11-1

(2,4-dichloro-6-phenyl-1,3,5-triazine (10.2 g, 45.3 mmol). (9-phenyl-9H-carbazol-2-yl)boronic acid (10.0 g, 34.8 mmol), PdCl₂(PPh₃)₄ (733 mg, 1.0 mmol), Na₂CO₃ (9.2 g, 87.1 mmol), 175 mL of THF, and 45 mL of H₂O were added to the flask and then stirred at 60° C. for 6 hours. After completion of the reaction, the mixture was cooled to room temperature and then the organic layers were extracted with ethyl acetate. The residual moisture was removed using magnesium sulfate and then dried. Next, it was separated by column chromatography to obtain compound 11-1 (13.5 g, yield: 69%).

2) Synthesis of Compound C-183

Compound 11-1 (6 g, 13.8 mmol), compound 1-2 (5.0 g, 11.5 mmol), Pd(PPh₃)₄ (400 mg, 0.35 mmol), K₂CO₃ (4.0 g, 28.8 mmol), 57 mL of toluene, 14 mL of EtOH, and 14 mL of H₂O were added to the flask and then stirred at 130° C. for 3 hours. After completion of the reaction, the mixture was cooled to room temperature and then the organic layers were extracted with ethyl acetate. The residual moisture was removed using magnesium sulfate and then dried. Next, it was separated by column chromatography to obtain compound C-183 (6.7 g, yield: 83%).

| | MW | M.P |
|---|---|---|
| C-183 | 765.9 | 292° C. |

### [Example 12] Synthesis of Compound C-185

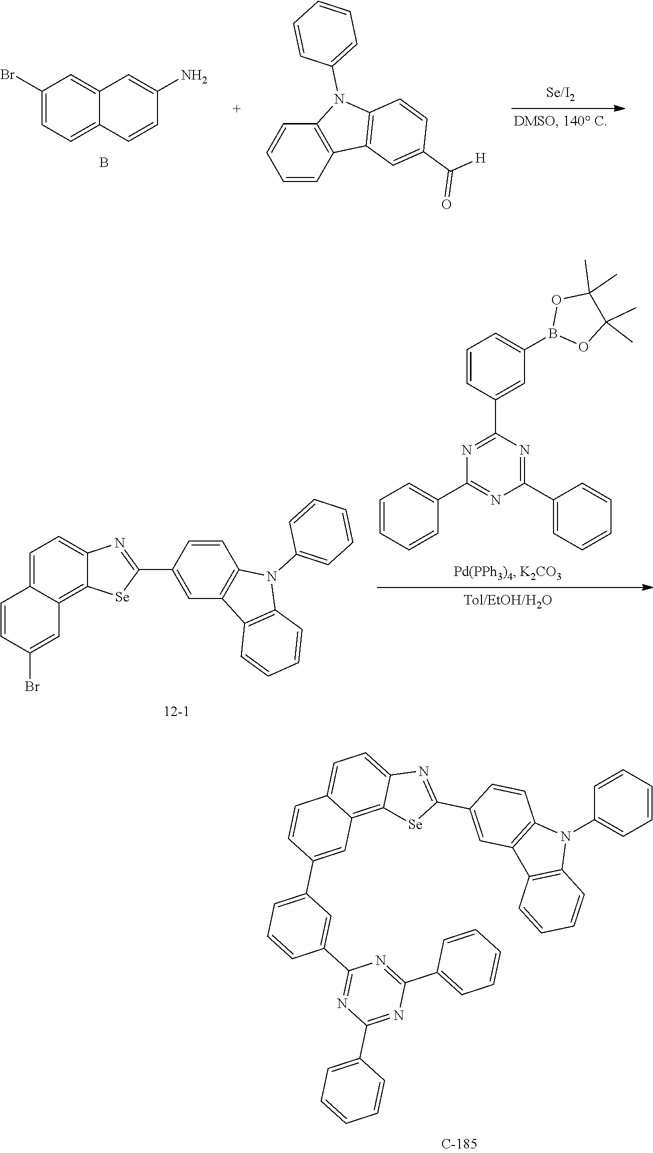

1) Synthesis of Compound 12-1

Compound B (10 g, 45.0 mmol), 9-phenyl-9H-carbazole-3-carbaldehyde (11.1 g, 40.9 mmol), Se (3.3 g, 40.9 mmol), I₂ (1.0 g, 4.1 mmol), and 205 mL of DMSO were added to the flask and then stirred at 140° C. for 6 hours. After completion of the reaction, the mixture was cooled to room temperature and then the organic layers were extracted with ethyl acetate. The residual moisture was removed using magnesium sulfate and then dried. Next, it was separated by column chromatography to obtain compound 12-1 (17.0 g, yield: 76%).

2) Synthesis of Compound C-185

Compound 12-1 (6 g, 10 mmol), 2,4-diphenyl-6-(3-(4,4,5,5-tetramethyl-1,3,2-dioxaborolan-2-yl)phenyl)-1,3,5-triazine (5.2 g, 11.9 mmol), Pd(PPh₃)₄ (627 mg, 0.5 mmol), K₂CO₃ (3.5 g, 25 mmol), 50 mL of toluene, 25 mL of EtOH, and 25 mL of H₂O were added to the flask and then stirred at 130° C. for 3 hours. After completion of the reaction, the mixture was cooled to room temperature and then the organic layers were extracted with ethyl acetate. The residual moisture was removed using magnesium sulfate and then dried. Next, it was separated by column chromatography to obtain compound C-185 (3.8 g, yield: 48%).

| | MW | M.P |
|---|---|---|
| C-185 | 780.0 | 311.7° C. |

Hereinafter, the preparation method of an organic electroluminescent device comprising the organic electroluminescent compound and/or the plurality of host materials of the present disclosure, and the device property thereof will be explained in order to understand the present disclosure in detail.

### [Device Examples 1 and 2] Preparation of Blue-Light Emitting OLEDs According to the Present Disclosure

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, compound HI-1 was introduced into a cell of the vacuum vapor deposition apparatus, and compound HT-1 was introduced into another cell. The two materials were evaporated at different rates and compound HI-1 was deposited in a doping amount of 3 wt % based on the total amount of compounds HI-1 and HT-1 to form a hole injection layer having a thickness of 10 nm. Next, compound HT-1 was deposited as a first hole transport layer having a thickness of 80 nm on the hole injection layer. Compound HT-2 was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows. Compound BH as a host was introduced into the one cell of two cells of the vacuum vapor deposition apparatus, and compound BD was introduced into another cell as a dopant, and then the dopant material was deposited in a doping amount of 2 wt % based on the total amounts of the host and the dopant to form a light-emitting layer having a thickness of 20 nm on the second hole transport layer. Next, the compound of the following Table 1 and EIL-1 as electron transport materials were deposited at a weight ratio of 50:50 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. Each compound used for all the materials were purified by vacuum sublimation under 10⁻⁶ torr.

### [Device Comparative Example 1] Preparation of an OLED with Comparative Compounds as Electron Transport Layer Materials

An OLED was produced in the same manner as in Device Example 1, except that compound A-1 was used as a material for the electron transport layer.

The driving voltage, the luminous efficiency, and the luminous color at a luminance of 1,000 nits, and the time taken for luminance to decrease from 100% to 80% at a luminance of 2,000 nits (lifespan; T80) of the OLEDs according to Device Examples 1 and 2 and Device Comparative Example 1 produced as described above, are measured, and the results thereof are shown in the following Table 1.

TABLE 1

| | Electron | Driving | Luminous | | |
|---|---|---|---|---|---|
| | Transport | Voltage | Efficiency | Luminous | Lifespan |
| | Layer | (V) | (cd/A) | Color | (T80, hrs) |
| Device | C-89: | 4.4 | 4.9 | Blue | 637 |
| Example 1 | EIL-1 | | | | |
| Device | C-95: | 4.6 | 3.8 | Blue | 666 |
| Example 2 | EIL-1 | | | | |
| Device | A-1: | 4.2 | 6.1 | Blue | 294 |
| Comparative | EIL-1 | | | | |
| Example 1 | | | | | |

From Table 1 above, it can be confirmed that the organic electroluminescent device including the organic electroluminescent compound according to the present disclosure as a material for an electron transport layer exhibits significantly improved lifespan characteristics compared to the organic electroluminescent device including the conventional compound in the electron transport layer.

The compounds used in Device Examples 1 and 2 and Device Comparative Example 1 are specifically shown in the following Table 2.

| | |
|---|---|
| Hole Injection Layer/ Hole Transport Layer | 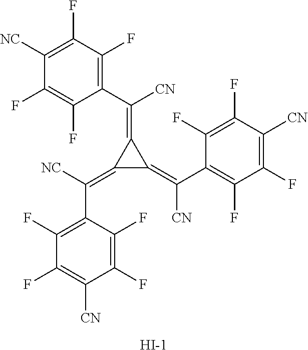 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 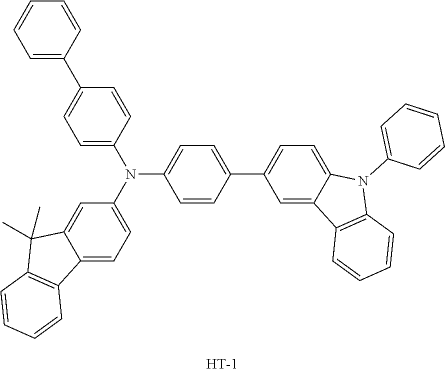 |
| | 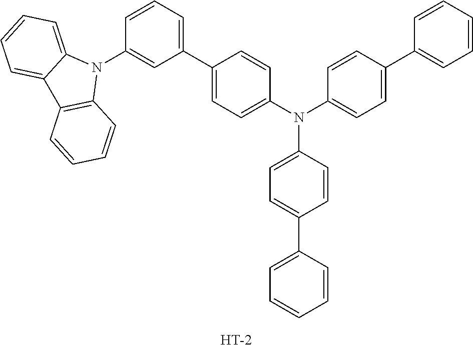 |
| Light-Emitting Layer | 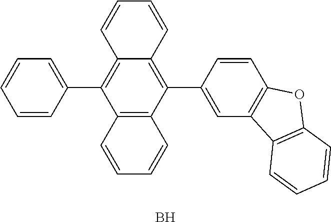 |
| | 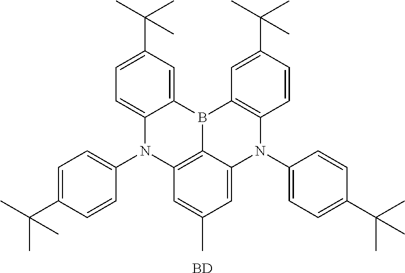 |
| Electron Transport Layer/ Electron Injection Layer | 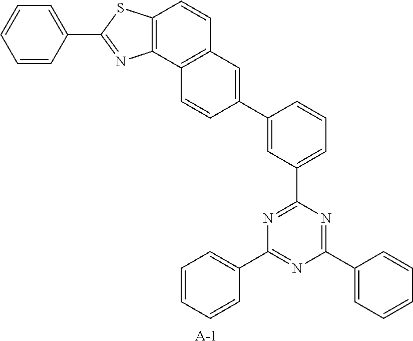 |
| | 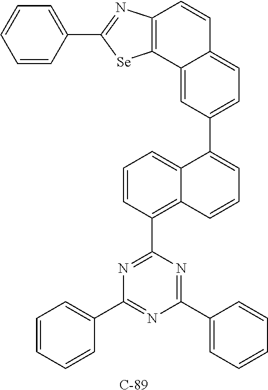 |
| | 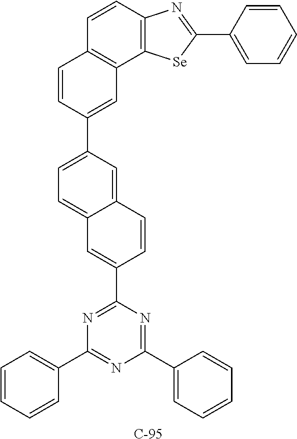 |
| | 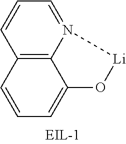 |

### [Device Examples 3 to 16] Preparation of Red-Light Emitting OLEDs According to the Present Disclosure

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropanol and then used. Thereafter, the ITO substrate was mounted on a substrate holder of a vacuum vapor deposition apparatus. Then, compound HI-1 as a first hole injection compound was introduced into a cell of the vacuum vapor deposition apparatus, and compound HT-1 as a first hole transport compound was introduced into another cell. The two materials were evaporated at different rates and the first hole injection compound was deposited in a doping amount of 3 wt % based on the total amount of the first hole injection and the first hole transport compounds to form a hole injection layer having a thickness of 10 nm. Next, compound HT-1 was deposited as a first hole transport layer having a thickness of 80 nm on the hole injection layer. Compound HT-3 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: each of the first host material and the second host material described in the following Table 3 were introduced into two cells of the vacuum vapor deposition apparatus as hosts, respectively, and compound D-39 was introduced into another cell as a dopant. The two host materials were evaporated at a rate of 1:1 and the dopant material was evaporated at a different rate, simultaneously, and was deposited in a doping amount of 3 wt % based on the total amount of the hosts and dopant to form a light-emitting layer having a thickness of 40 nm on the second hole transport layer. Next, compounds ETL-1 and EIL-1 as electron transport materials in another two cells were deposited at a rate of 1:1 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus. OLEDs were produced.

### [Device Comparative Example 2] Preparation of an OLED Comprising the Comparative Compound as a Host

An OLED was produced in the same manner as in Device Example 3, except that compound A-1 was used as a first host material of the light-emitting layer.

### [Device Comparative Examples 3 to 9] Preparation of OLEDs Comprising the Comparative Compound as a Host

OLEDs were produced in the same manner as in Device Example 3, except that a host material of the following Table 3 was used alone as the host material of the light-emitting layer.

The driving voltage, the luminous efficiency, and the luminous color at a luminance of 1,000 nits, and the time taken for luminance to decrease from 100% to 95% at a luminance of 10,000 nits (lifespan; T95) of the OLEDs according to Device Examples 3 to 16 and Device Comparative Examples 2 to 9 produced as described above, are measured, and the results thereof are shown in the following Table 3.

TABLE 3

| | | | Driving | Luminous | | |
|---|---|---|---|---|---|---|
| | First Host | Second Host | Voltage | Efficiency | Luminous | Lifespan |
| | Material | Material | (V) | (cd/A) | Color | (T95, hrs) |
| Device | C-111 | H1-129 | 3.2 | 37.2 | Red | 73.3 |
| Example 3 | | | | | | |
| Device | C-64 | H1-129 | 3.2 | 36.5 | Red | 28.0 |
| Example 4 | | | | | | |
| Device | C-89 | H1-129 | 3.0 | 36.0 | Red | 140 |
| Example 5 | | | | | | |
| Device | C-175 | H4-111 | 2.9 | 34.4 | Red | 502 |
| Example 6 | | | | | | |
| Device | C-175 | H4-21 | 2.9 | 31.4 | Red | 416 |
| Example 7 | | | | | | |
| Device | C-175 | H4-112 | 2.9 | 35.8 | Red | 719 |
| Example 8 | | | | | | |
| Device | C-175 | H3-1 | 2.9 | 35.2 | Red | 542 |
| Example 9 | | | | | | |
| Device | C-175 | H3-3 | 2.8 | 33.2 | Red | 466 |
| Example 10 | | | | | | |
| Device | C-89 | H3-3 | 3.0 | 35.4 | Red | 446 |
| Example 11 | | | | | | |
| Device | C-89 | H3-3 | 2.9 | 33.9 | Red | 346 |
| Example 12 | | | | | | |
| Device | C-175 | H2-11 | 2.9 | 33.3 | Red | 482 |
| Example 13 | | | | | | |
| Device | C-175 | H2-14 | 2.9 | 32.7 | Red | 655 |
| Example 14 | | | | | | |
| Device | C-89 | H2-11 | 3.0 | 33.6 | Red | 376 |
| Example 15 | | | | | | |
| Device | C-89 | H2-14 | 3.0 | 33.9 | Red | 470 |
| Example 16 | | | | | | |
| Device | A-1 | H1-129 | 3.5 | 36.1 | Red | 3.1 |
| Comparative | | | | | | |
| Example 2 | | | | | | |
| Device | — | H4-111 | 4.8 | 5.3 | Red | 6.1 |
| Comparative | | | | | | |
| Example 3 | | | | | | |
| Device | — | H4-21 | 6.2 | 3.3 | Red | 2.3 |
| Comparative | | | | | | |
| Example 4 | | | | | | |
| Device | — | H4-112 | 4.6 | 6.5 | Red | 9.4 |
| Comparative | | | | | | |
| Example 5 | | | | | | |
| Device | — | H3-1 | 4.2 | 5.7 | Red | 14.3 |
| Comparative | | | | | | |
| Example 6 | | | | | | |
| Device | — | H3-3 | 4.6 | 3.8 | Red | 10.4 |
| Comparative | | | | | | |
| Example 7 | | | | | | |
| Device | — | H2-11 | 4.4 | 8.8 | Red | 34 |
| Comparative | | | | | | |
| Example 8 | | | | | | |
| Device | — | H2-14 | 4.2 | 7.4 | Red | 34 |
| Comparative | | | | | | |
| Example 9 | | | | | | |

From Table 3 above, it can be seen that the organic electroluminescent devices according to Device Examples 3 to 16 exhibit low driving voltage, high efficiency, and long lifespan characteristics compared to Device Comparative Examples 2 to 9.

### [Device Example 17] Preparation of Red-Light Emitting OLED According to the Present Disclosure

An OLED was produced in the same manner as in Device Example 3, except that compound C-175 was used as a first host material of the light-emitting layer.

The driving voltage, the luminous efficiency, and the luminous color at a luminance of 1,000 nits, and the time taken for luminance to decrease from 100% to 95% at a luminance of 10,000 nits (lifespan; T95) of the OLED according to Device Example 17 produced as described above, are measured, and the results thereof are shown in the following Table 4, and compared with the characteristics of Device Comparative Example 2 above.

TABLE 4

| | | | Driving | Luminous | | |
|---|---|---|---|---|---|---|
| | First Host | Second Host | Voltage | Efficiency | Luminous | Lifespan |
| | Material | Material | (V) | (cd/A) | Color | (T95, hrs) |
| Device | C-175 | H1-129 | 2.9 | 34.5 | Red | 232 |
| Example 17 | | | | | | |
| Device | A-1 | H1-129 | 3.5 | 36.1 | Red | 3.1 |
| Comparative | | | | | | |
| Example 2 | | | | | | |

From Table 4 above, it can be confirmed that the organic electroluminescent device according to Device Example 17 exhibits low driving voltage and in particular, significantly improved lifespan characteristics when comparing with Device Comparative Example 2.

### [Device Examples 18 and 19] Preparation of Red-Light Emitting OLEDs According to the Present Disclosure

OLEDs were produced in the same manner as in Device Example 3, except that a compound of the following Table 5 was used alone as a first host material of the light-emitting layer.

### [Device Comparative Example 10] Preparation of an OLED Comprising the Comparative Compound as a Host

An OLED was produced in the same manner as in Device Example 18, except that a compound of the following Table 5 was used alone as a first host material of the light-emitting layer.

The driving voltage and the luminous color at a luminance of 1,000 nits, and the time taken for luminance to decrease from 100% to 95% at a luminance of 10,000 nits (lifespan; T95) of the OLEDs according to Device Examples 18 and 19 and Device Comparative Example 10 produced as described above, are measured, and the results thereof are shown in the following Table 5.

TABLE 5

| | First | Second | Driving | | |
|---|---|---|---|---|---|
| | Host | Host | Voltage | Luminous | Lifespan |
| | Material | Material | (V) | Color | (T95, hrs) |
| Device | C-183 | — | 3.0 | Red | 8.2 |
| Example 18 | | | | | |
| Device | C-185 | — | 3.0 | Red | 15.1 |
| Example 19 | | | | | |
| Device | A-1 | — | 4.8 | Red | 0.2 |
| Comparative | | | | | |
| Example 10 | | | | | |

From Table 5 above, it can be confirmed that the organic electroluminescent devices according to Device Examples 18 and 19 exhibit low driving voltage and in particular, significantly improved lifespan characteristics when comparing with Device Comparative Example 10.

The compounds used in Device Examples 3 to 19 and Device Comparative Examples 2 to 10 are specifically shown in the following Table 6.

| | |
|---|---|
| Hole Injection Layer/ Hole Transport Layer | 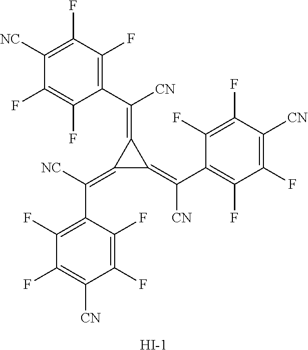 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 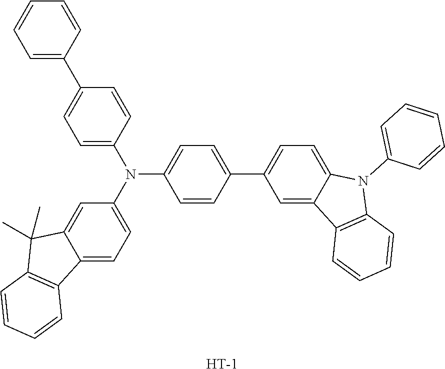 |
| | 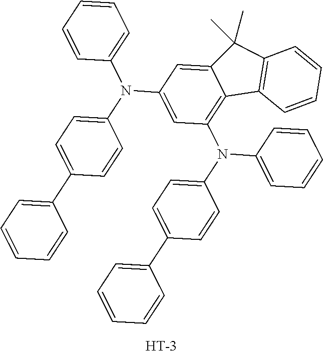 |
| Light-Emitting Layer | 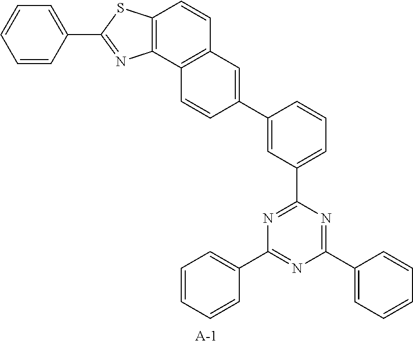 |
| | 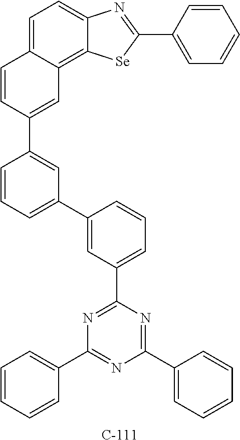 [SMILES: c1ccc(-c2nc(-c3ccccc3)nc(-c3cccc(-c4cccc(-c5ccc6ccc7nc(-c8ccccc8)[se]c7c6c5)c4)c3)n2)cc1 ; "C-111"] |
| | 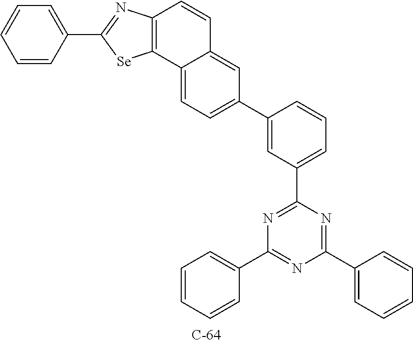 |
| | 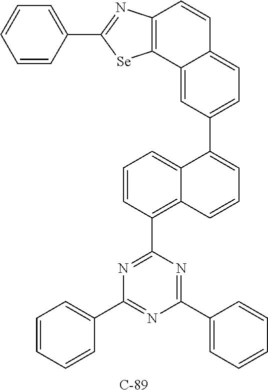 |
| | 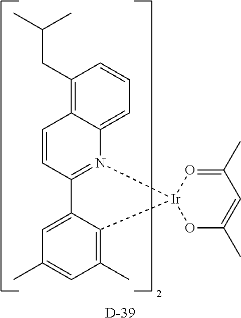 |
| | 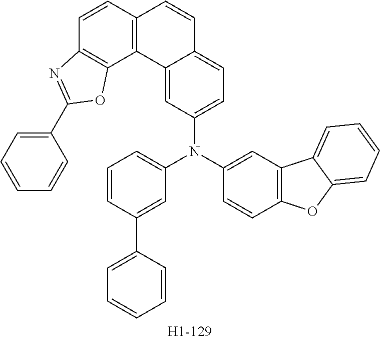 [SMILES: c1ccc(-c2cccc(N(c3ccc4oc5ccccc5c4c3)c3ccc4ccc5ccc6nc(-c7ccccc7)oc6c5c4c3)c2)cc1] |
| | 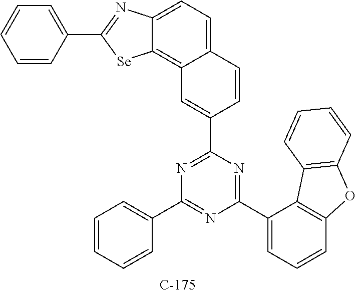 |
| | 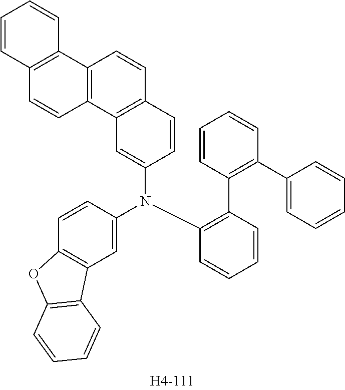 [SMILES: c1ccc(-c2ccccc2-c2ccccc2N(c2ccc3ccc4c5ccccc5ccc4c3c2)c2ccc3oc4ccccc4c3c2)cc1] |
| | 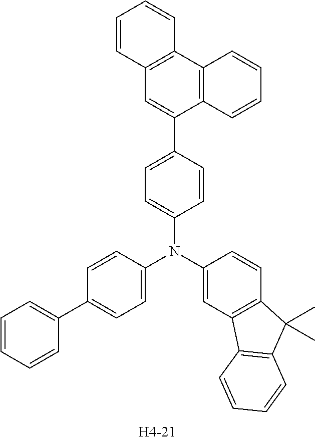 [SMILES: CC1(C)c2ccccc2-c2cc(N(c3ccc(-c4ccccc4)cc3)c3ccc(-c4cc5ccccc5c5ccccc45)cc3)ccc21] |
| | 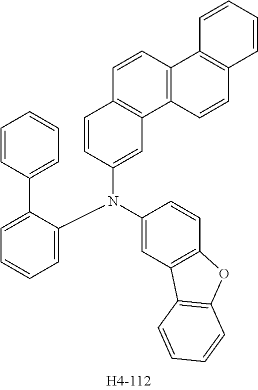 [SMILES: c1ccc(-c2ccccc2N(c2ccc3ccc4c5ccccc5ccc4c3c2)c2ccc3oc4ccccc4c3c2)cc1] |
| | 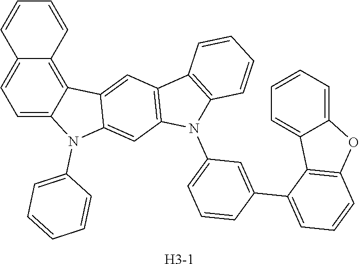 [SMILES: c1ccc(-n2c3cc4c(cc3c3c5ccccc5ccc32)c2ccccc2n4-c2cccc(-c3cccc4oc5ccccc5c34)c2)cc1 ; "H3-1"] |
| | 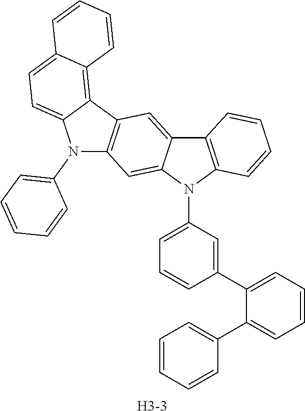 |
| | 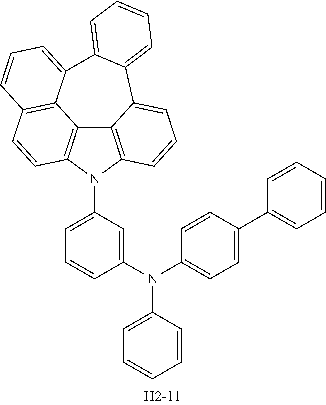 [SMILES: c1ccc(-c2ccc(N(c3ccccc3)c3cccc(-n4c5cccc6c5c5c7c(cccc7ccc54)-c4ccccc4-6)c3)cc2)cc1] |
| | 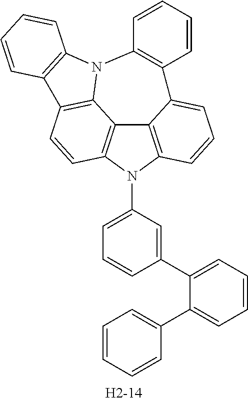 |
| | 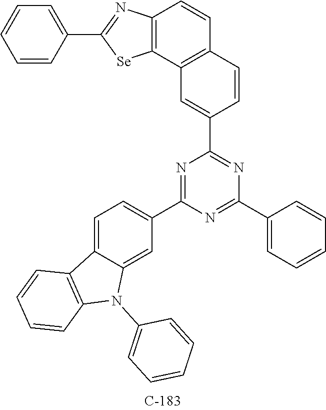 [SMILES: c1ccc(-c2nc(-c3ccc4ccc5nc(-c6ccccc6)[se]c5c4c3)nc(-c3ccc4c5ccccc5n(-c5ccccc5)c4c3)n2)cc1] |
| | 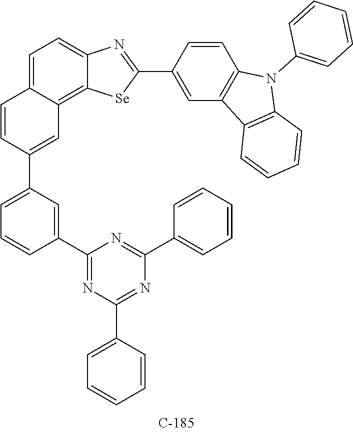 [SMILES: c1ccc(-c2nc(-c3ccccc3)nc(-c3cccc(-c4ccc5ccc6nc(-c7ccc8c(c7)c7ccccc7n8-c7ccccc7)[se]c6c5c4)c3)n2)cc1] |
| Electron Transport Layer/ Electron Injection Layer | 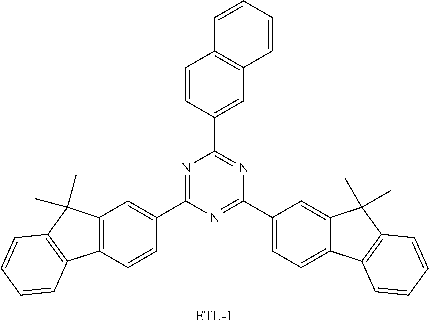 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 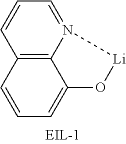 |

1. An organic electroluminescent compound represented by the following formula 1: 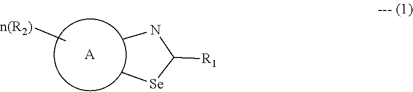
wherein,
ring A is a naphthalene ring;
R₁ represents a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, -L′₁-SiR′₁R′₂R′₃, -L′₁-GeR′₁R′₂R′₃, or -L′₂NR′₄R′₅;
R₂ represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, -L′₁-SiR′₁R′₂R′₃, or -L′₂-NR′₄R′₅; or may be linked to the adjacent substituents to form a ring(s);
L′₁ and L′₂ each independently represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
R′₁ to R′₅ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
n is an integer of 0 to 6.

2. The organic electroluminescent compound according to claim 1, wherein the formula 1 is represented by any one of the following formulas 1-1 to 1-3: 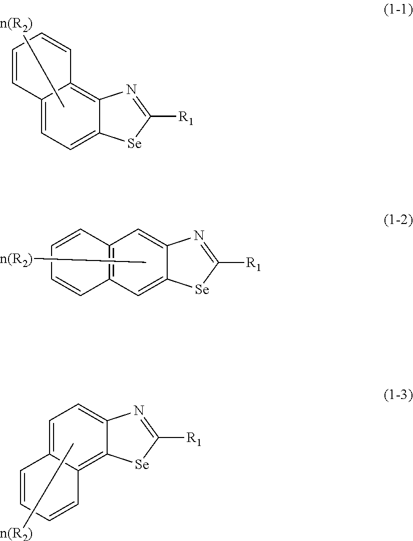
wherein,
R₁, R₂, and n are as defined in claim 1.

3. The organic electroluminescent compound according to claim 1, wherein the formula 1 is represented by any one of the following formulas 1-4 to 1-6: 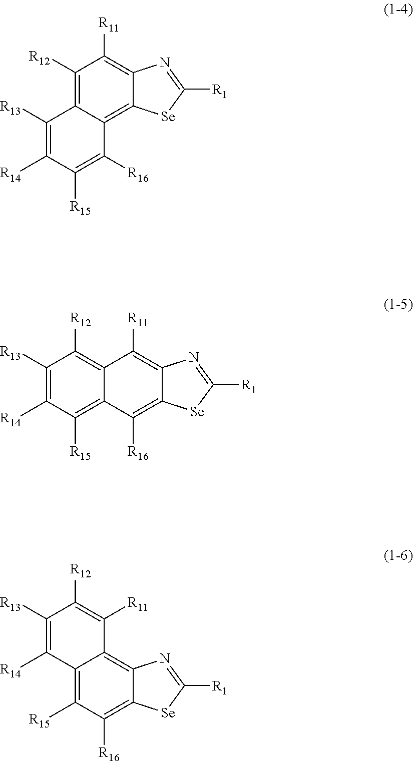
wherein,
R₁ is as defined in claim 1;
R₁₁ to R₁₆ are each independently as defined as R₂ in claim 1; provided that, at least one of R₁ and R₁₁ to R₁₆ represent(s) -L₁-HAr;
L₁ represents a single bond, a substituted or unsubstituted (C6-C30)arylene, a substituted or unsubstituted (3- to 30-membered)heteroarylene, or a substituted or unsubstituted (C3-C30)cycloalkylene; and
HAr represents a substituted or unsubstituted nitrogen containing (3- to 30-membered)heteroaryl.

4. The organic electroluminescent compound according to claim 3, wherein HAr is a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted benzoxazolyl, a substituted or unsubstituted pyrazolyl, a substituted or unsubstituted naphthyridinyl, or a substituted or unsubstituted benzothienopyrimidinyl.

5. The organic electroluminescent compound according to claim 3, wherein -L₁-HAr is represented by the following formula 1′: 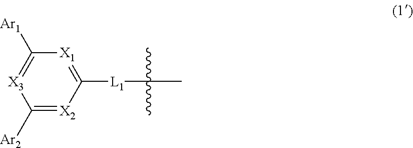
wherein,
X₁ to X₃ each independently represent N or CH; provided that, at least one of X₁ to X₃ represent(s) N;
L₁ is as defined as L₁ in claim 3; and
Ar₁ and Ar₂ each independently represent a substituted or unsubstituted (C6-C30)aryl or a substituted or unsubstituted (3- to 30-membered)heteroaryl.

6. The organic electroluminescent compound according to claim 1, wherein the substituents of the substituted alkyl(ene), the substituted alkenyl, the substituted aryl(ene), the substituted heteroaryl(ene), the substituted cycloalkyl(ene), and the substituted alkoxy, each independently represent at least one selected from the group consisting of deuterium; halogen; cyano; carboxyl; nitro; hydroxy; (C1-C30)alkyl; halo(C1-C30)alkyl; (C2-C30)alkenyl; (C2-C30)alkynyl; (C1-C30)alkoxy; (C1-C30)alkylthio; (C3-C30)cycloalkyl; (C3-C30)cycloalkenyl; (3- to 7-membered)heterocycloalkyl; (C6-C30)aryloxy; (C6-C30)arylthio; (3- to 30-membered)heteroaryl unsubstituted or substituted with at least one (C6-C30)aryl; (C6-C30)aryl unsubstituted or substituted with at least one of (C1-C30)alkyl and (3- to 30-membered)heteroaryl; tri(C1-C30)alkylsilyl; tri(C6-C30)arylsilyl; di(C1-C30)alkyl(C6-C30)arylsilyl; (C1-C30)alkyldi(C6-C30)arylsilyl; tri(C6-C30)arylgermanyl; amino; mono- or di-(C1-C30)alkylamino; mono- or di-(C2-C30)alkenylamino; mono- or di-(C6-C30)arylamino unsubstituted or substituted with (C1-C30)alkyl; mono- or di-(3- to 30-membered)heteroarylamino; (C1-C30)alkyl(C2-C30)alkenylamino; (C1-C30)alkyl(C6-C30)arylamino; (C1-C30)alkyl(3- to 30-membered)heteroarylamino; (C2-C30)alkenyl(C6-C30)arylamino; (C2-C30)alkenyl(3- to 30-membered)heteroarylamino; (C6-C30)aryl(3- to 30-membered)heteroarylamino; (C1-C30)alkylcarbonyl; (C1-C30)alkoxycarbonyl; (C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl.

7. The organic electroluminescent compound according to claim 1, wherein the compound represented by the formula 1 is selected from the following compounds:

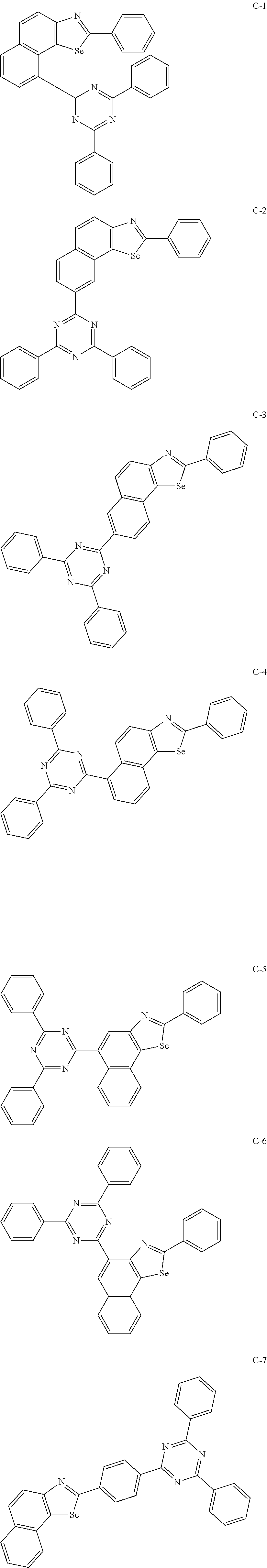

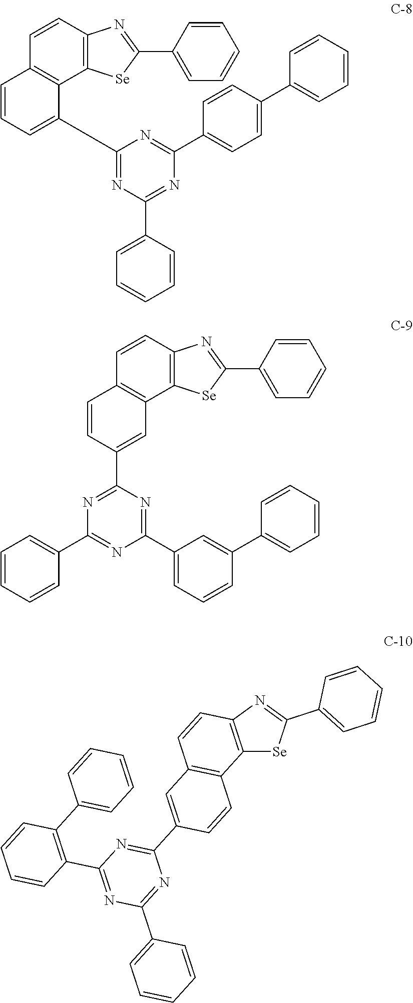

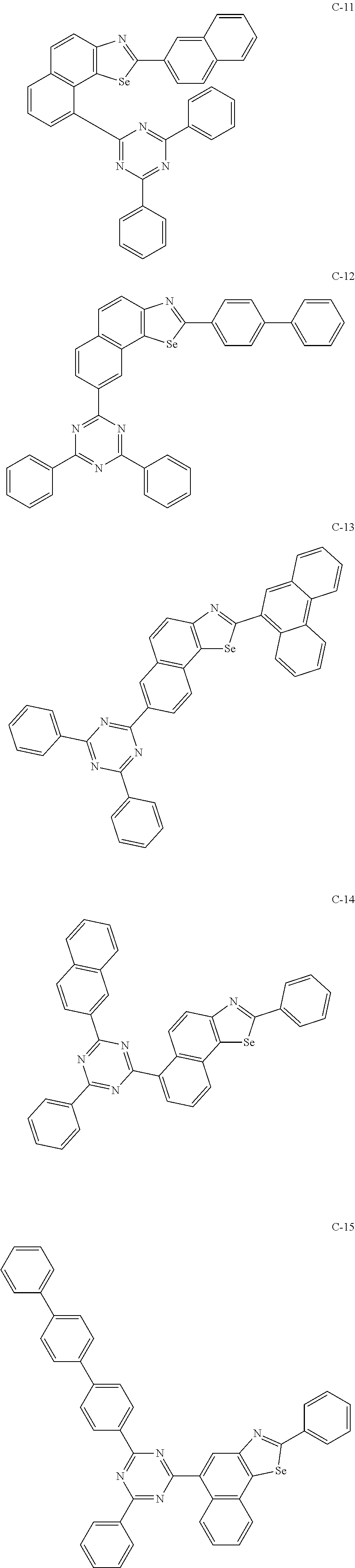 [SMILES: c1ccc(-c2nc(-c3ccccc3)nc(-c3cccc4ccc5nc(-c6ccc7ccccc7c6)[se]c5c34)n2)cc1 ; c1ccc(-c2ccc(-c3nc4ccc5ccc(-c6nc(-c7ccccc7)nc(-c7ccccc7)n6)cc5c4[se]3)cc2)cc1 ; c1ccc(-c2nc(-c3ccccc3)nc(-c3ccc4c(ccc5nc(-c6cc7ccccc7c7ccccc67)[se]c54)c3)n2)cc1 ; c1ccc(-c2nc(-c3ccc4ccccc4c3)nc(-c3cccc4c3ccc3nc(-c5ccccc5)[se]c34)n2)cc1 ; c1ccc(-c2ccc(-c3ccc(-c4nc(-c5ccccc5)nc(-c5cc6nc(-c7ccccc7)[se]c6c6ccccc56)n4)cc3)cc2)cc1]

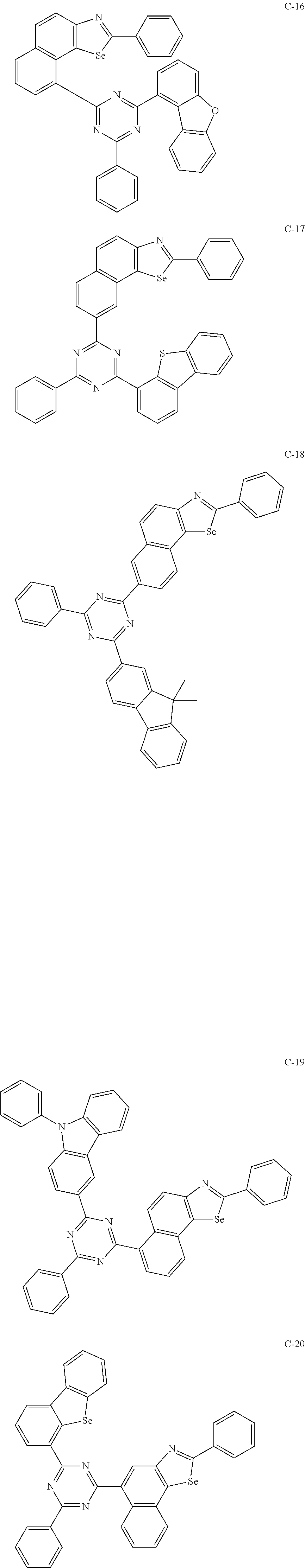

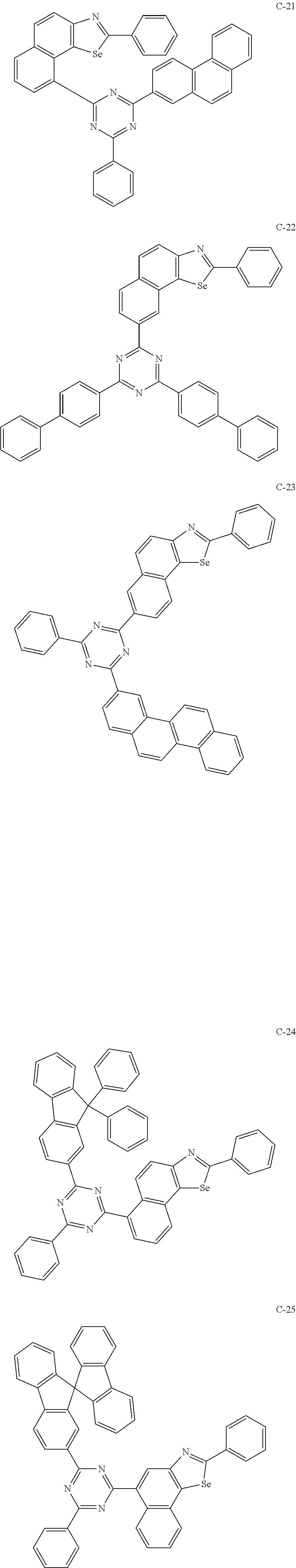 [SMILES: c1ccc(-c2nc(-c3ccc4c(ccc5ccccc54)c3)nc(-c3cccc4ccc5nc(-c6ccccc6)[se]c5c34)n2)cc1 ; c1ccc(-c2ccc(-c3nc(-c4ccc(-c5ccccc5)cc4)nc(-c4ccc5ccc6nc(-c7ccccc7)[se]c6c5c4)n3)cc2)cc1 ; c1ccc(-c2nc(-c3ccc4c(ccc5nc(-c6ccccc6)[se]c54)c3)nc(-c3ccc4ccc5c6ccccc6ccc5c4c3)n2)cc1 ; c1ccc(-c2nc(-c3ccc4c(c3)C(c3ccccc3)(c3ccccc3)c3ccccc3-4)nc(-c3cccc4c3ccc3nc(-c5ccccc5)[se]c34)n2)cc1 ; c1ccc(-c2nc(-c3ccc4c(c3)C3(c5ccccc5-c5ccccc53)c3ccccc3-4)nc(-c3cc4nc(-c5ccccc5)[se]c4c4ccccc34)n2)cc1]

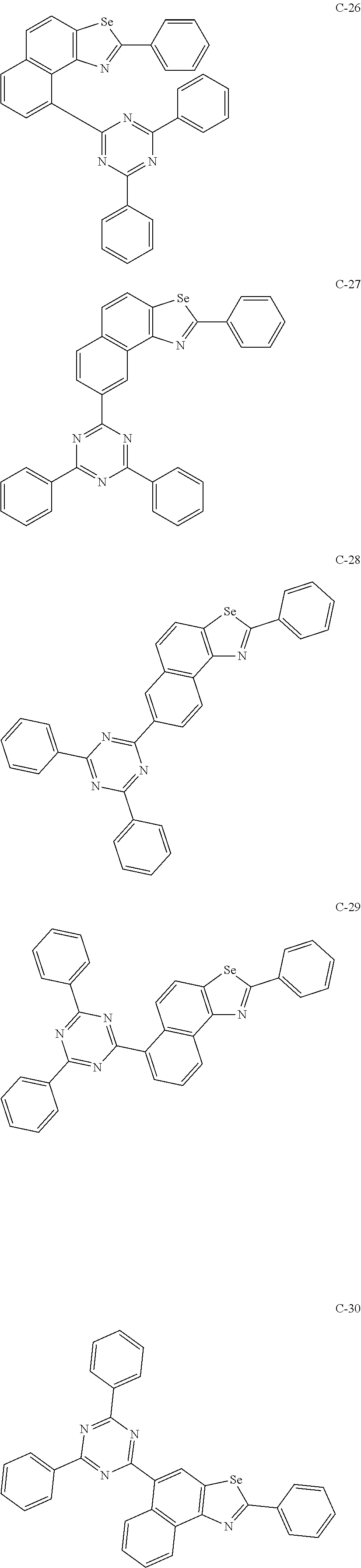

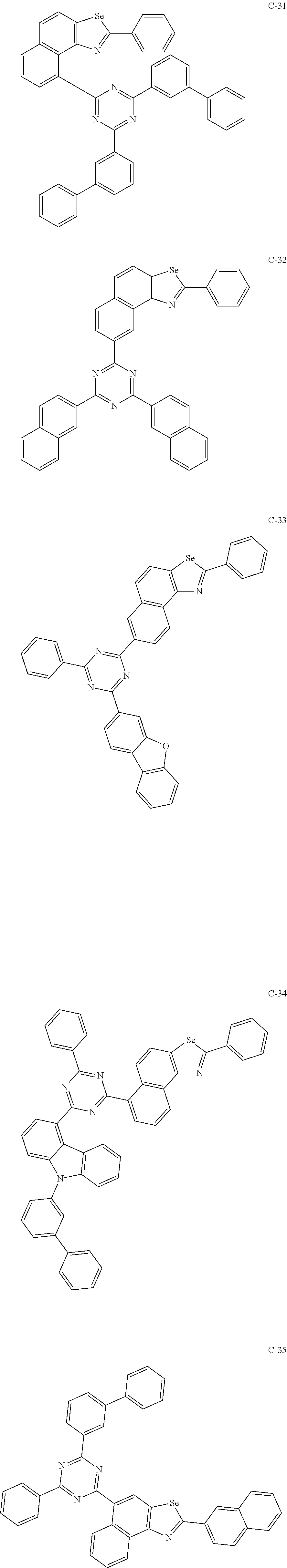 [SMILES: c1ccc(-c2cccc(-c3nc(-c4cccc(-c5ccccc5)c4)nc(-c4cccc5ccc6[se]c(-c7ccccc7)nc6c45)n3)c2)cc1 ; c1ccc(-c2nc3c(ccc4ccc(-c5nc(-c6ccc7ccccc7c6)nc(-c6ccc7ccccc7c6)n5)cc43)[se]2)cc1 ; c1ccc(-c2nc(-c3ccc4c(ccc5[se]c(-c6ccccc6)nc54)c3)nc(-c3ccc4c(c3)oc3ccccc34)n2)cc1 ; c1ccc(-c2cccc(-n3c4ccccc4c4c(-c5nc(-c6ccccc6)nc(-c6cccc7c6ccc6[se]c(-c8ccccc8)nc67)n5)cccc43)c2)cc1 ; c1ccc(-c2cccc(-c3nc(-c4ccccc4)nc(-c4cc5[se]c(-c6ccc7ccccc7c6)nc5c5ccccc45)n3)c2)cc1]

[chemical structure]

[chemical structure]

[chemical structure]

[chemical structure]

[chemical structure]

[chemical structure]

[chemical structure]

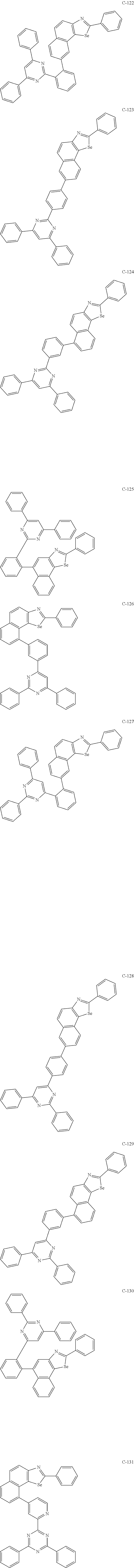

[chemical structure]

[chemical structure]

[chemical structure]

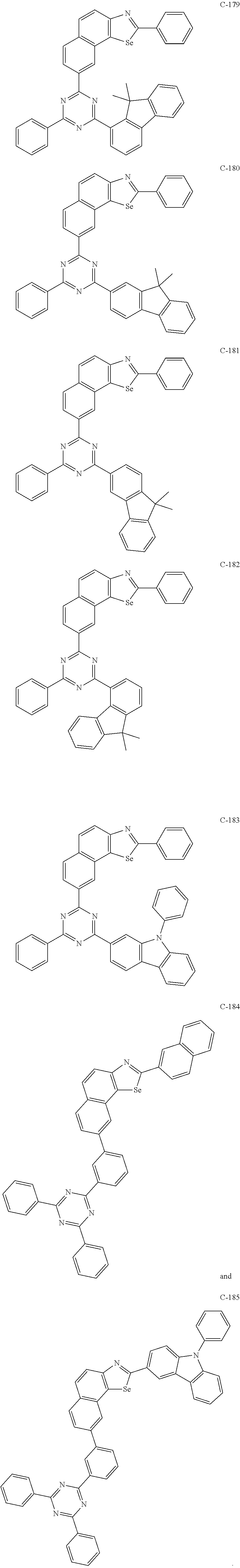

8. A plurality of host materials comprising a first host material and a second host material, wherein the first host material comprises an organic electroluminescent compound according to claim 1 and the second host material comprises an organic electroluminescent compound represented by the following formula 2: 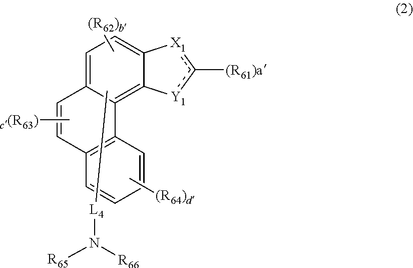
wherein,
X₁ and Y₁ each independently represent —N═, —NR₆₇—, —O— or —S—; provided that, one of X₁ and Y₁ represents —N═, and the other of X₁ and Y₁ represents —NR₆₇—, —O— or —S—;
R₆₁ represents a substituted or unsubstituted (C6-C30)aryl, or a substituted or unsubstituted (3- to 30-membered)heteroaryl;
R₆₂ to R₆₄ and R₆₇ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and an (C6-C30) aromatic ring, or -L₃″-N(Ar₃″)(Ar₄″); or may be linked to the adjacent substituents to form a ring(s);
L₃″ represents a single bond, a substituted or unsubstituted (C6-C30)arylene, or a substituted or unsubstituted (3- to 30-membered)heteroarylene;
Ar₃″ and Ar₄″ each independently represent hydrogen, a substituted or unsubstituted (C1-C30)alkyl, a substituted or unsubstituted (C2-C30)alkenyl, a substituted or unsubstituted fused ring of (C3-C30) aliphatic ring and an (C6-C30) aromatic ring, a substituted or unsubstituted (C6-C30)aryl, or a substituted or unsubstituted (3- to 30-membered)heteroaryl;
L₄ represents a single bond, a substituted or unsubstituted (C6-C30)arylene, or a substituted or unsubstituted (3- to 30-membered)heteroarylene;
R₆₅ and R₆₆ each independently represent a substituted or unsubstituted (C6-C30)aryl, or a substituted or unsubstituted (3- to 30-membered)heteroaryl;
a′ is 1, b′ and c′ each independently represent an integer of 1 or 2, and d′ represents an integer of 1 to 4; and
when b′ to d′ are 2 or more, each of R₆₂ to R₆₄ may be the same or different.

9. The plurality of host materials according to claim 8, wherein the compound represented by formula 2 is selected from the following compounds:

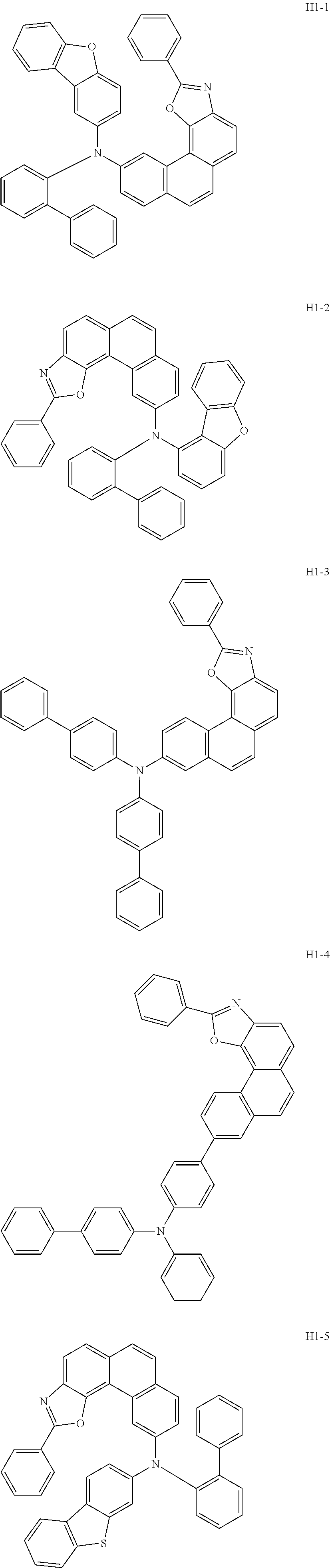 [SMILES: c1ccc(-c2nc3ccc4ccc5ccc(N(c6ccc7oc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1 ; C1=CC(N(c2ccc(-c3ccccc3)cc2)c2ccc(-c3ccc4c(ccc5ccc6nc(-c7ccccc7)oc6c54)c3)cc2)=CCC1 ; c1ccc(-c2nc3ccc4ccc5ccc(N(c6ccc7c(c6)sc6ccccc67)c6ccccc6-c6ccccc6)cc5c4c3o2)cc1]

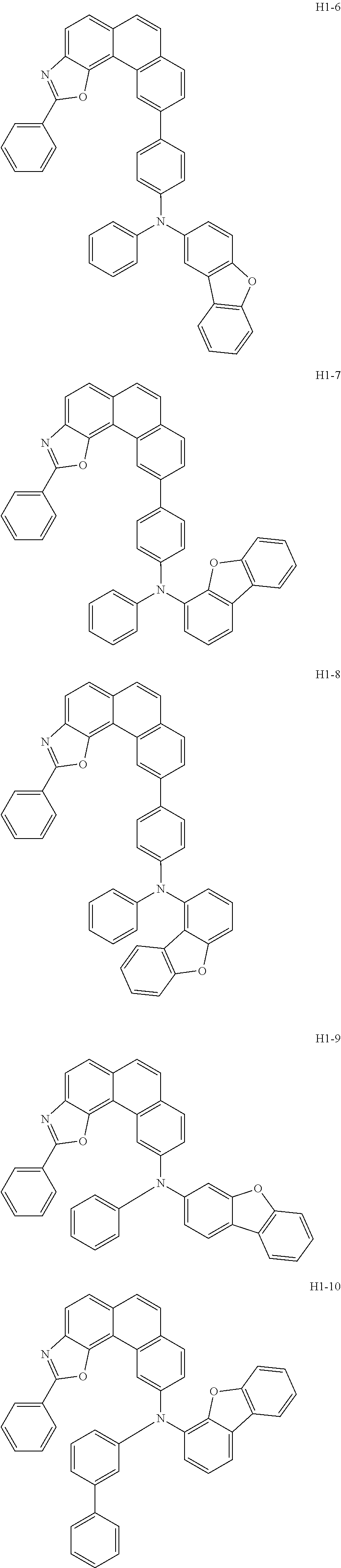 [SMILES: c1ccc(-c2nc3ccc4ccc5ccc(-c6ccc(N(c7ccccc7)c7ccc8oc9ccccc9c8c7)cc6)cc5c4c3o2)cc1 ; c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccccc6)c6ccc7c(c6)oc6ccccc67)cc5c4c3o2)cc1 ; c1ccc(-c2cccc(N(c3ccc4ccc5ccc6nc(-c7ccccc7)oc6c5c4c3)c3cccc4c3oc3ccccc34)c2)cc1]

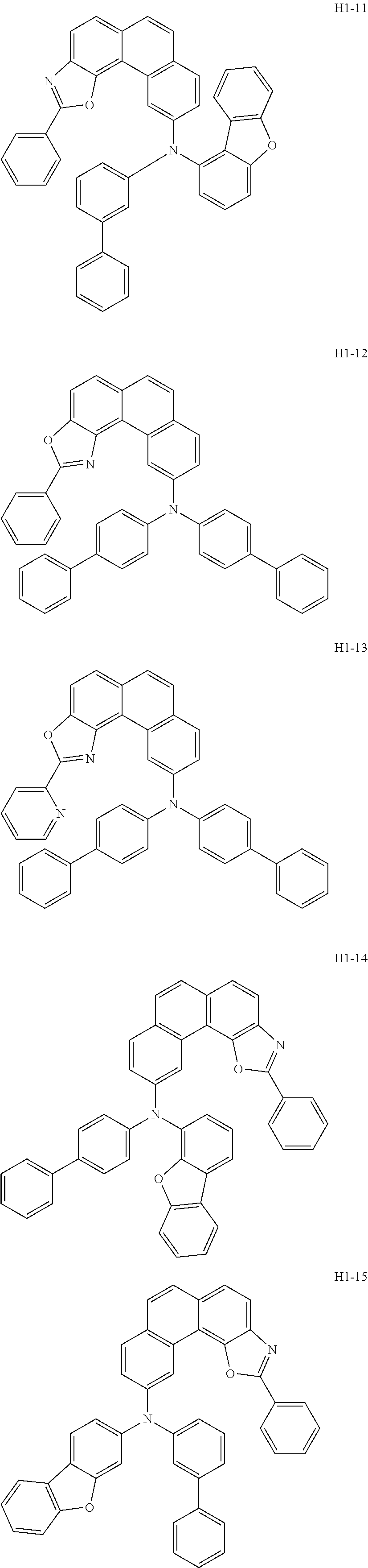

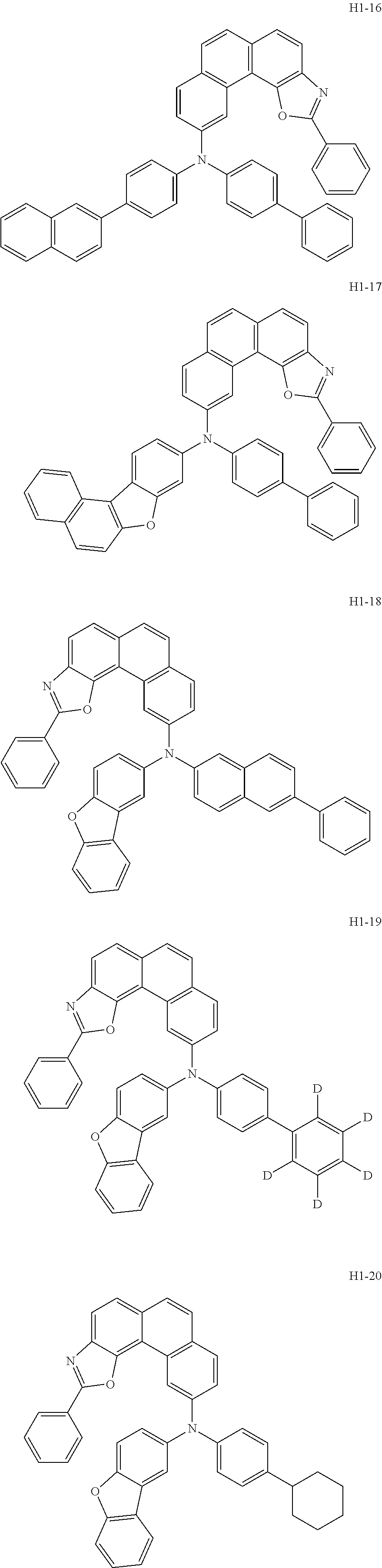 [SMILES: c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1 ; [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2nc3ccc4ccc5ccc(N(c6ccc(C7CCCCC7)cc6)c6ccc7oc8ccccc8c7c6)cc5c4c3o2)cc1]

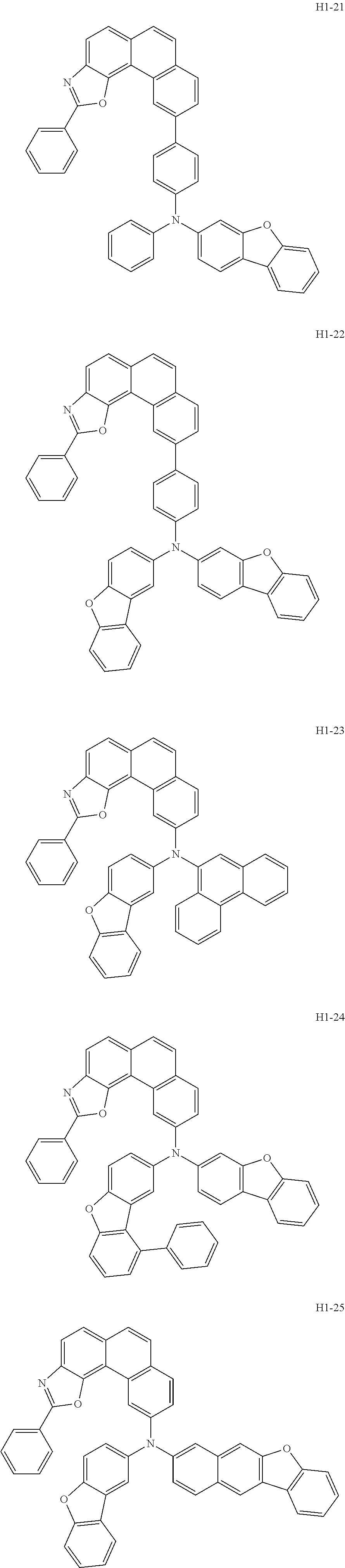 [SMILES: c1ccc(-c2nc3ccc4ccc5ccc(-c6ccc(N(c7ccccc7)c7ccc8c(c7)oc7ccccc78)cc6)cc5c4c3o2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1]

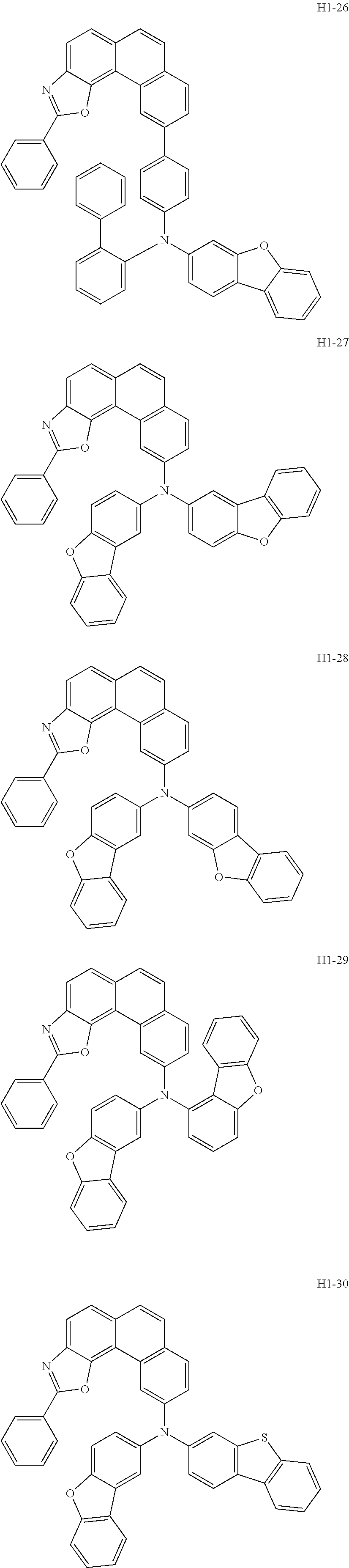 [SMILES: c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1 ; c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1]

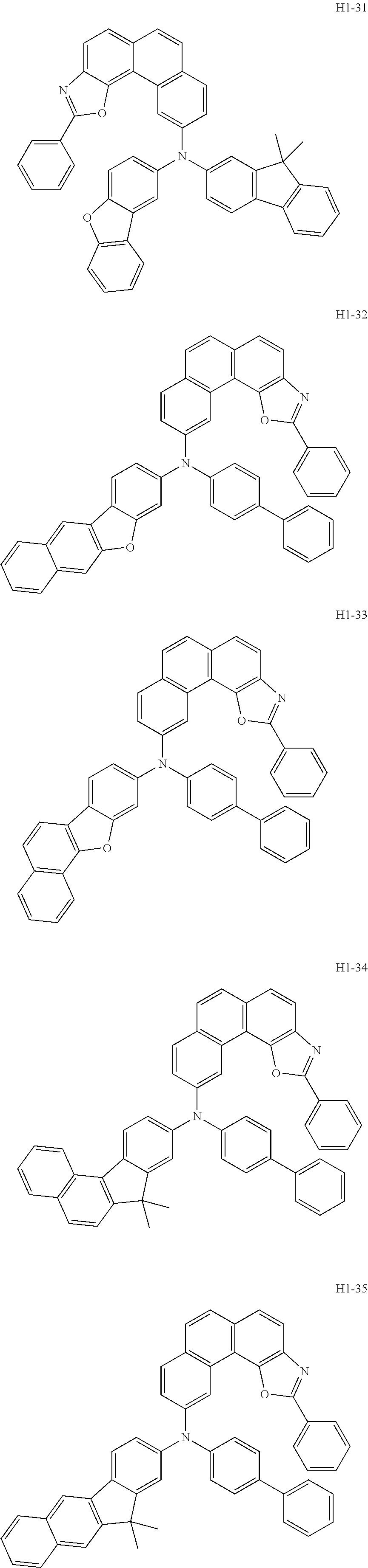 [SMILES: CC1(C)c2ccccc2-c2ccc(N(c3ccc4oc5ccccc5c4c3)c3ccc4ccc5ccc6nc(-c7ccccc7)oc6c5c4c3)cc21 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21]

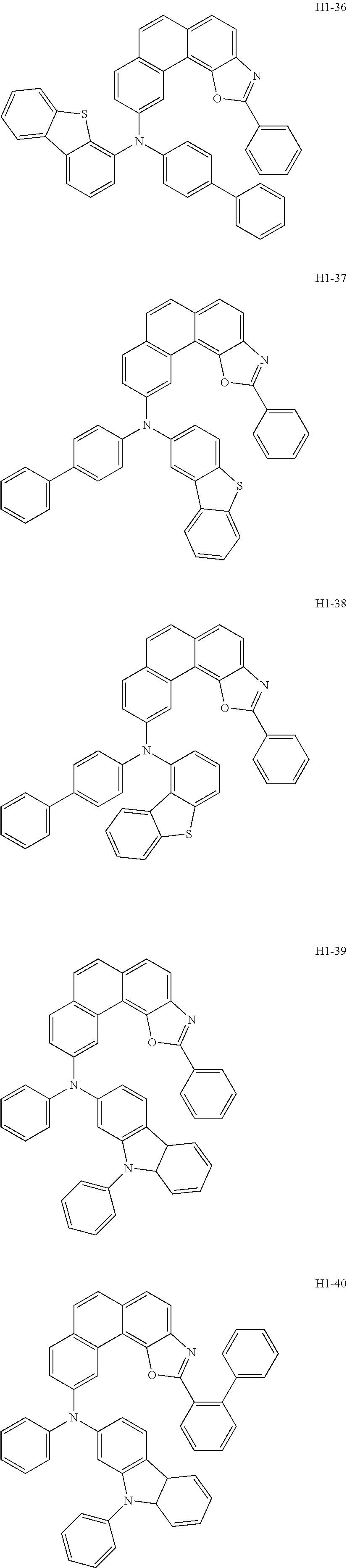 [SMILES: c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1 ; C1=CC2c3ccc(N(c4ccccc4)c4ccc5ccc6ccc7nc(-c8ccccc8-c8ccccc8)oc7c6c5c4)cc3N(c3ccccc3)C2C=C1]

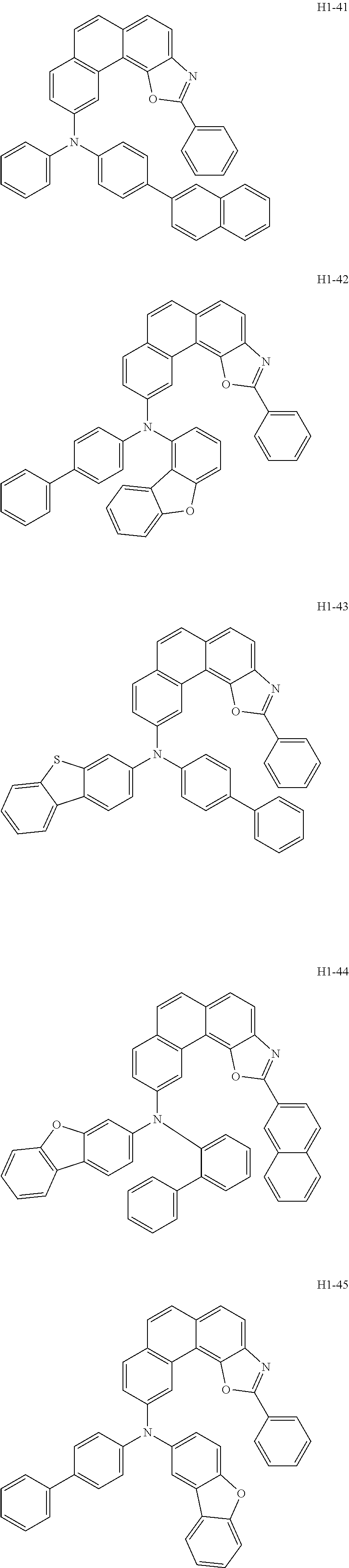 [SMILES: c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccccc2N(c2ccc3c(c2)oc2ccccc23)c2ccc3ccc4ccc5nc(-c6ccc7ccccc7c6)oc5c4c3c2)cc1 ; c1ccc(-c2ccc(N(c3ccc4oc5ccccc5c4c3)c3ccc4ccc5ccc6nc(-c7ccccc7)oc6c5c4c3)cc2)cc1]

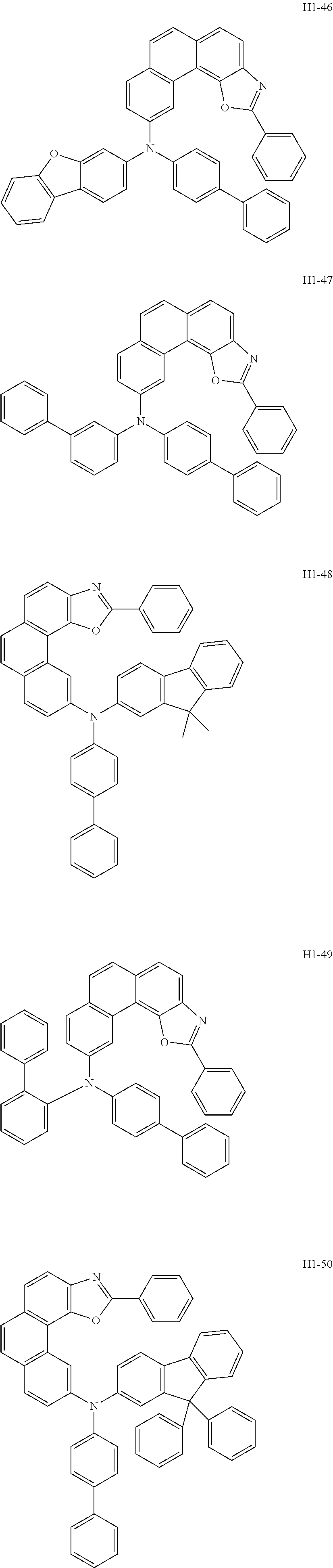 [SMILES: c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1 ; c1ccc(-c2ccc(N(c3ccc4c(c3)C(c3ccccc3)(c3ccccc3)c3ccccc3-4)c3ccc4ccc5ccc6nc(-c7ccccc7)oc6c5c4c3)cc2)cc1]

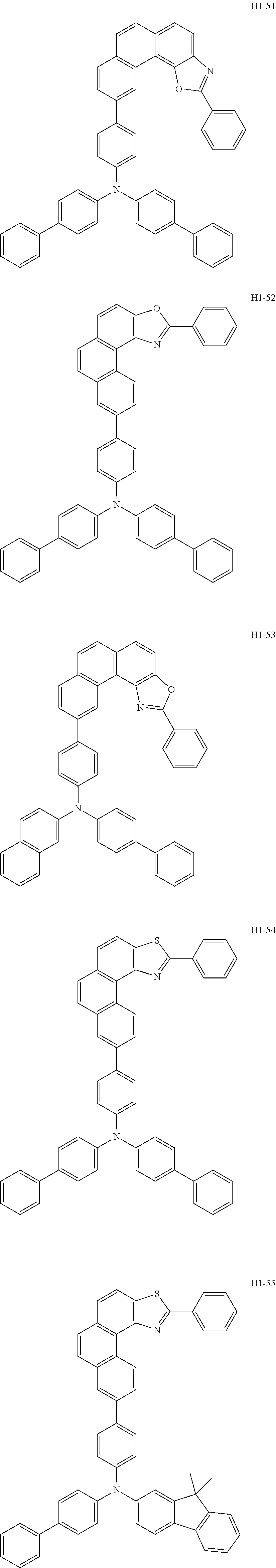 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21]

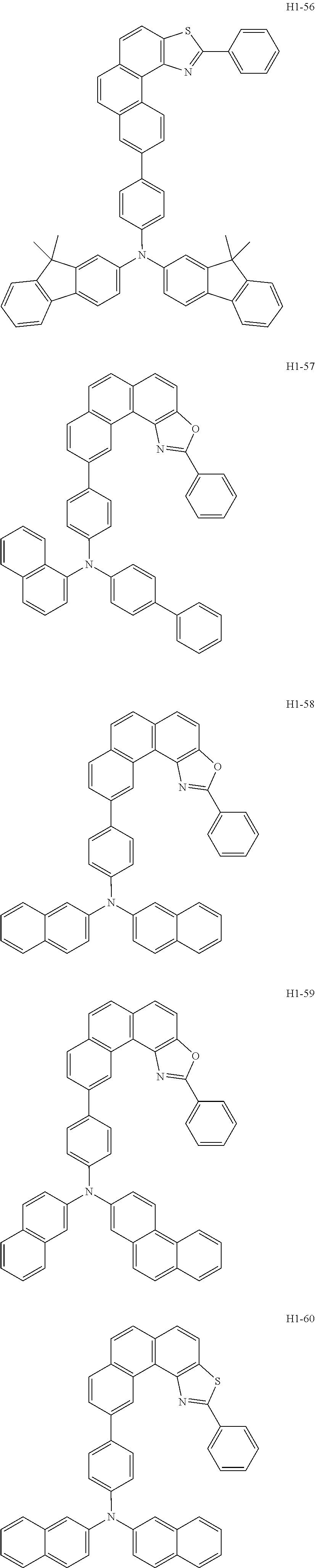 [SMILES: CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1 ; c1ccc(-c2nc3c(ccc4ccc5ccc(-c6ccc(N(c7ccc8ccccc8c7)c7ccc8ccccc8c7)cc6)cc5c43)s2)cc1]

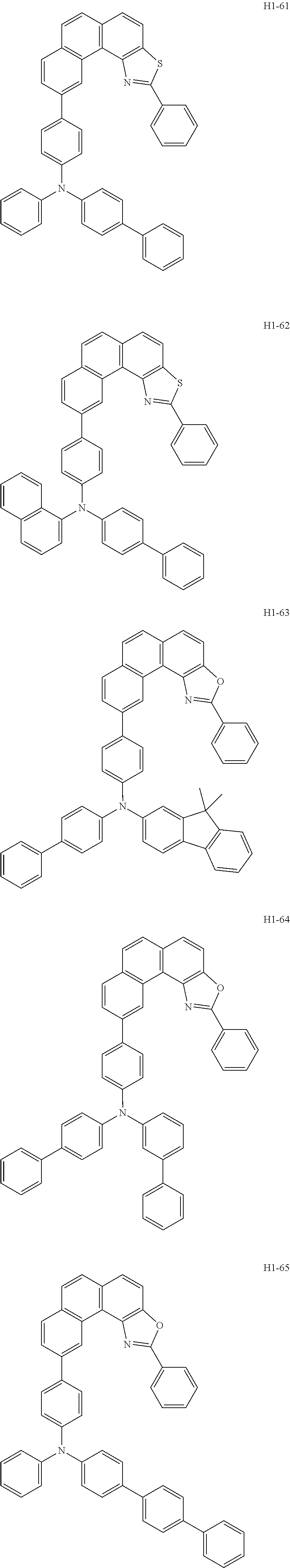 [SMILES: c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

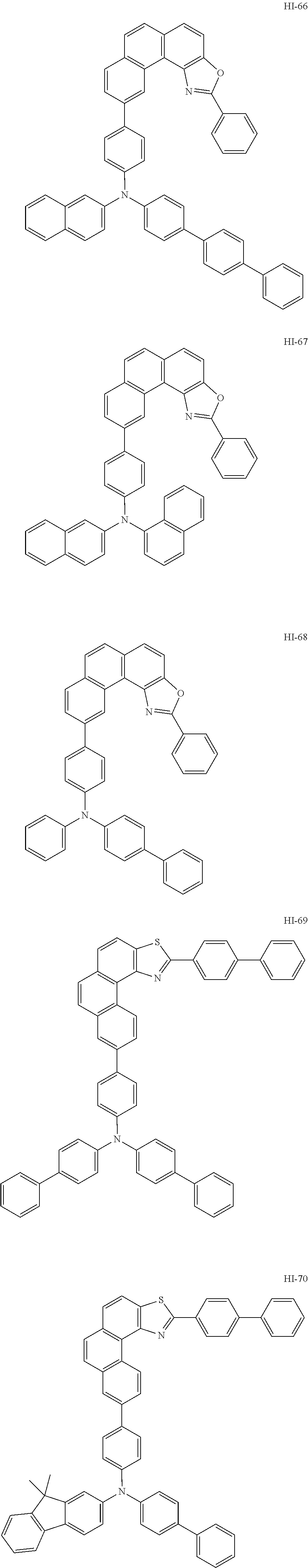 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21]

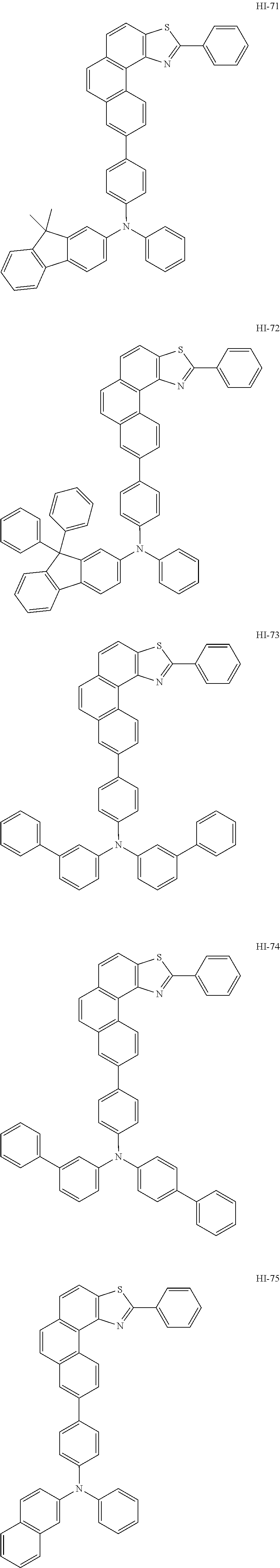 [SMILES: CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2nc3c(ccc4ccc5cc(-c6ccc(N(c7ccccc7)c7ccc8c(c7)C(c7ccccc7)(c7ccccc7)c7ccccc7-8)cc6)ccc5c43)s2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1]

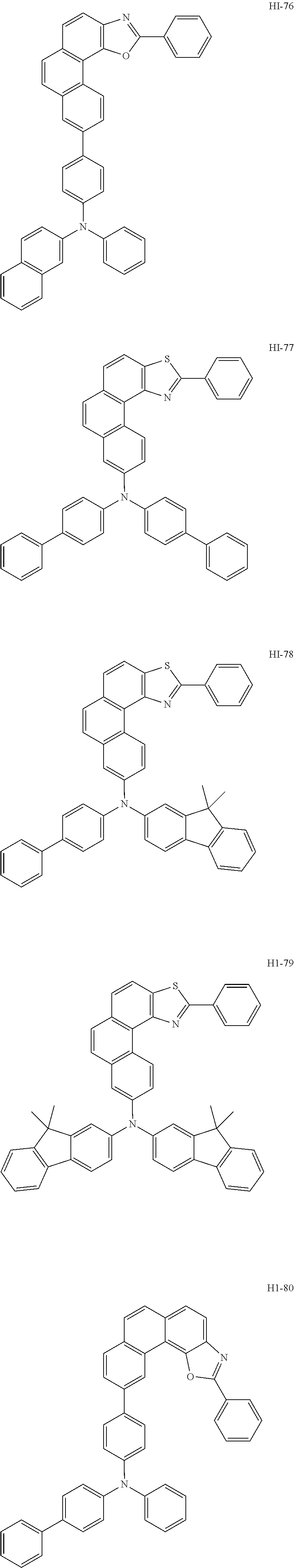 [SMILES: c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1 ; CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

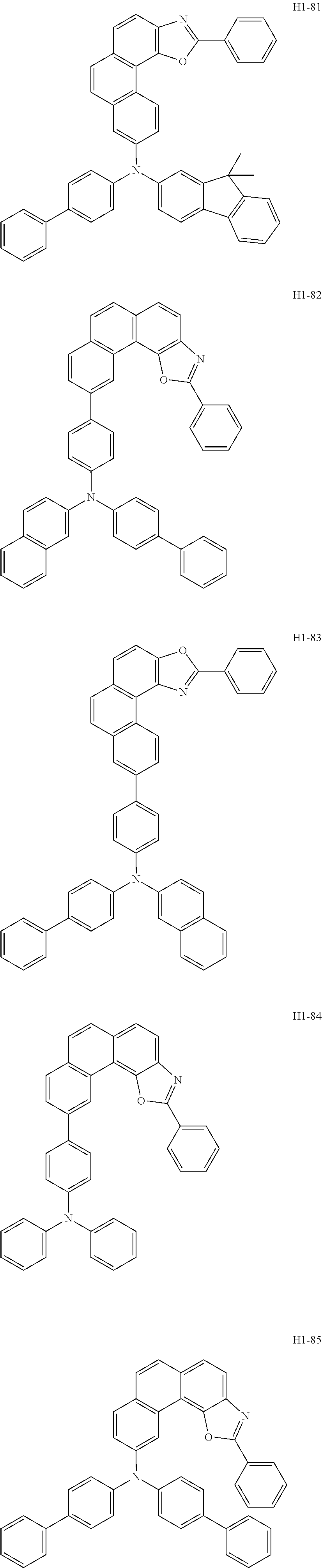 [SMILES: CC1(C)c2ccccc2-c2ccc(N(c3ccc(-c4ccccc4)cc3)c3ccc4c(ccc5ccc6nc(-c7ccccc7)oc6c54)c3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1]

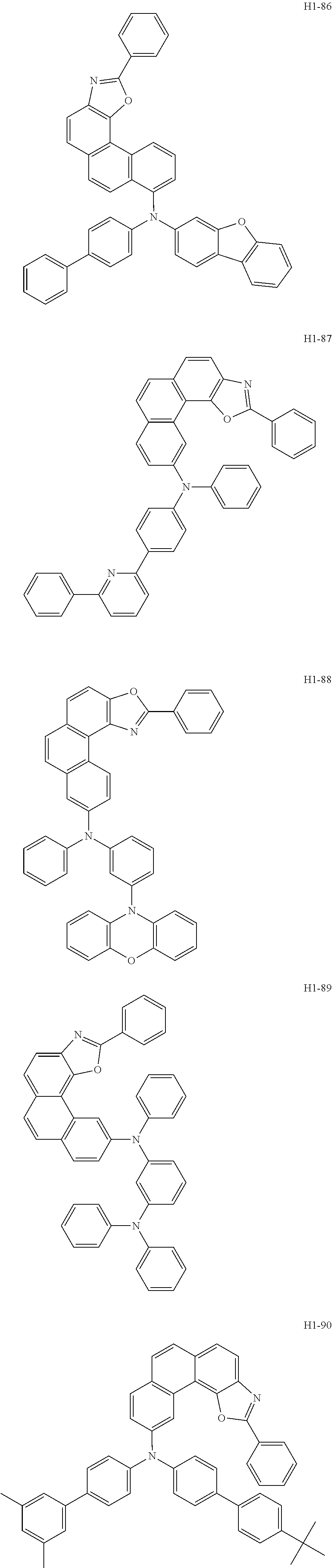 [SMILES: c1ccc(-c2ccc(N(c3ccc4c(c3)oc3ccccc34)c3cccc4c3ccc3ccc5nc(-c6ccccc6)oc5c34)cc2)cc1 ; c1ccc(-c2cccc(-c3ccc(N(c4ccccc4)c4ccc5ccc6ccc7nc(-c8ccccc8)oc7c6c5c4)cc3)n2)cc1 ; c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1 ; Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1]

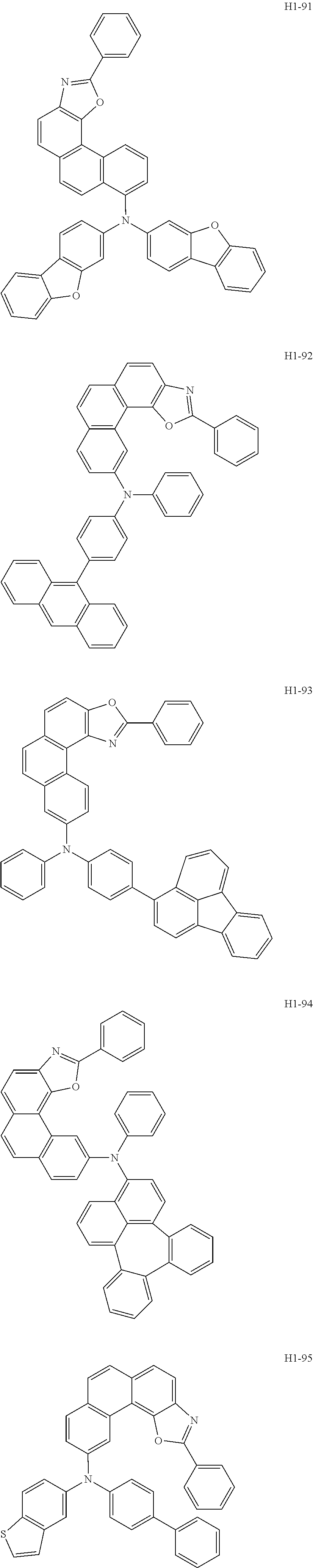 [SMILES: c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2nc3ccc4ccc5ccc(N(c6ccccc6)c6ccc(-c7c8ccccc8cc8ccccc78)cc6)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1 ; c1ccc(-c2nc3ccc4ccc5ccc(N(c6ccccc6)c6ccc7c8c(cccc68)-c6ccccc6-c6ccccc6-7)cc5c4c3o2)cc1 ; c1ccc(-c2ccc(N(c3ccc4sccc4c3)c3ccc4ccc5ccc6nc(-c7ccccc7)oc6c5c4c3)cc2)cc1]

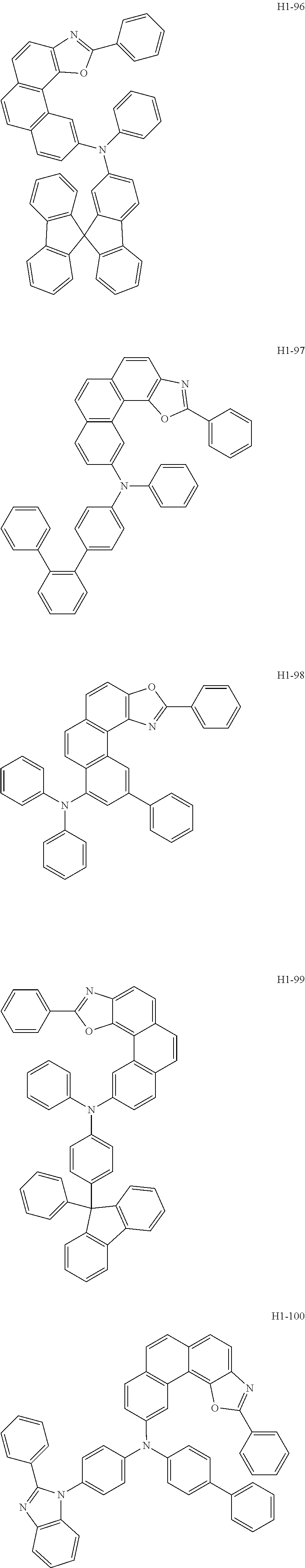 [SMILES: c1ccc(-c2nc3ccc4ccc5ccc(N(c6ccccc6)c6ccc7c(c6)C6(c8ccccc8-c8ccccc86)c6ccccc6-7)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1]

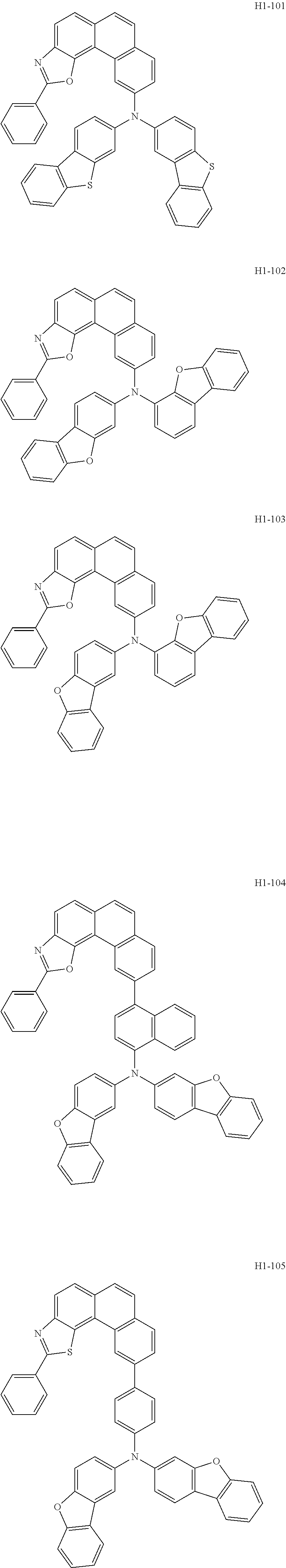 [SMILES: c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7c(c6)oc6ccccc67)c6cccc7c6oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3s2)cc1]

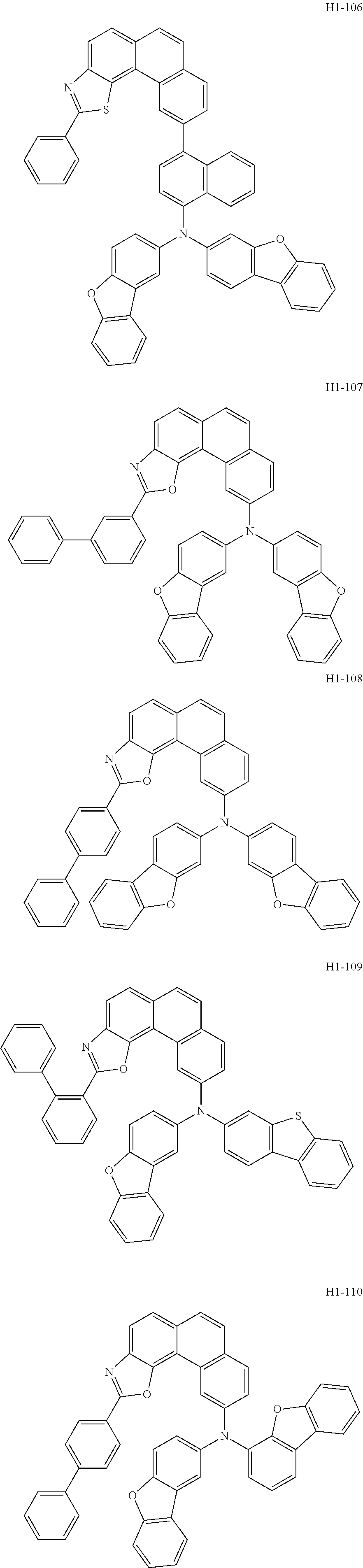 [SMILES: c1ccc(-c2nc3ccc4ccc5ccc(-c6ccc(N(c7ccc8c(c7)oc7ccccc78)c7ccc8oc9ccccc9c8c7)c7ccccc67)cc5c4c3s2)cc1 ; c1ccc(-c2cccc(-c3nc4ccc5ccc6ccc(N(c7ccc8oc9ccccc9c8c7)c7ccc8oc9ccccc9c8c7)cc6c5c4o3)c2)cc1 ; c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1 ; c1ccc(-c2ccc(-c3nc4ccc5ccc6ccc(N(c7ccc8oc9ccccc9c8c7)c7cccc8c7oc7ccccc78)cc6c5c4o3)cc2)cc1]

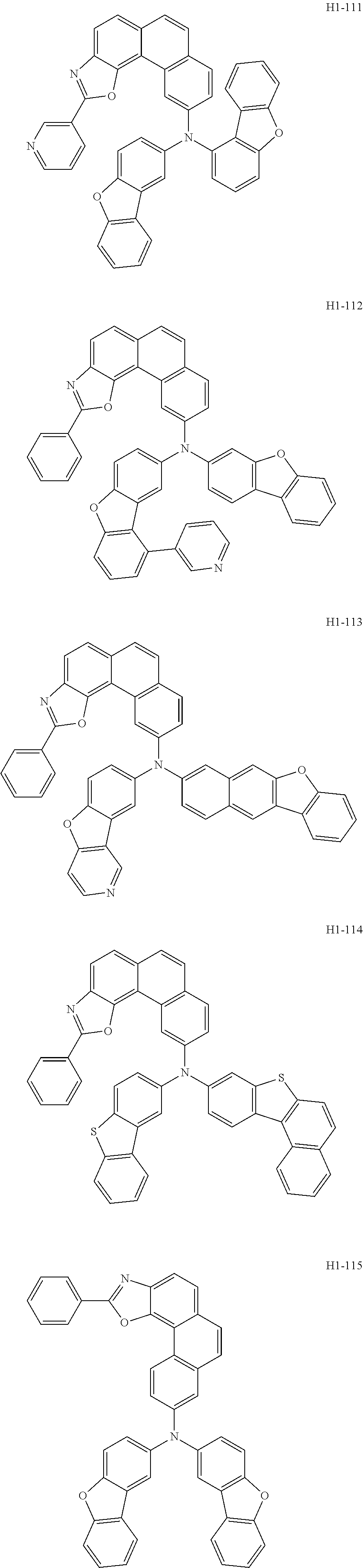 [SMILES: c1cncc(-c2nc3ccc4ccc5ccc(N(c6ccc7oc8ccccc8c7c6)c6cccc7oc8ccccc8c67)cc5c4c3o2)c1 ; c1ccc(-c2nc3ccc4ccc5ccc(N(c6ccc7c(c6)oc6ccccc67)c6ccc7oc8cccc(-c9cccnc9)c8c7c6)cc5c4c3o2)cc1 ; c1ccc(-c2nc3ccc4ccc5ccc(N(c6ccc7cc8c(cc7c6)oc6ccccc68)c6ccc7oc8ccncc8c7c6)cc5c4c3o2)cc1 ; c1ccc(-c2nc3ccc4ccc5ccc(N(c6ccc7c(c6)sc6ccc8ccccc8c67)c6ccc7sc8ccccc8c7c6)cc5c4c3o2)cc1 ; c1ccc(-c2nc3ccc4ccc5cc(N(c6ccc7oc8ccccc8c7c6)c6ccc7oc8ccccc8c7c6)ccc5c4c3o2)cc1]

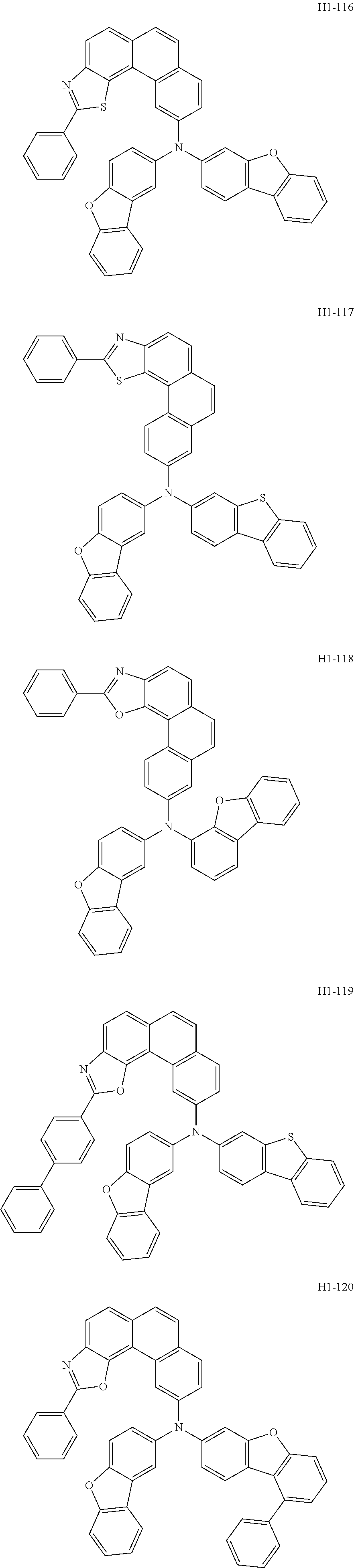 [SMILES: c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2nc3ccc4ccc5cc(N(c6ccc7c(c6)sc6ccccc67)c6ccc7oc8ccccc8c7c6)ccc5c4c3s2)cc1 ; c1ccc(-c2nc3ccc4ccc5cc(N(c6ccc7oc8ccccc8c7c6)c6cccc7c6oc6ccccc67)ccc5c4c3o2)cc1 ; c1ccc(-c2ccc(-c3nc4ccc5ccc6ccc(N(c7ccc8c(c7)sc7ccccc78)c7ccc8oc9ccccc9c8c7)cc6c5c4o3)cc2)cc1 ; c1ccc(-c2nc3ccc4ccc5ccc(N(c6ccc7c(c6)oc6cccc(-c8ccccc8)c67)c6ccc7oc8ccccc8c7c6)cc5c4c3o2)cc1]

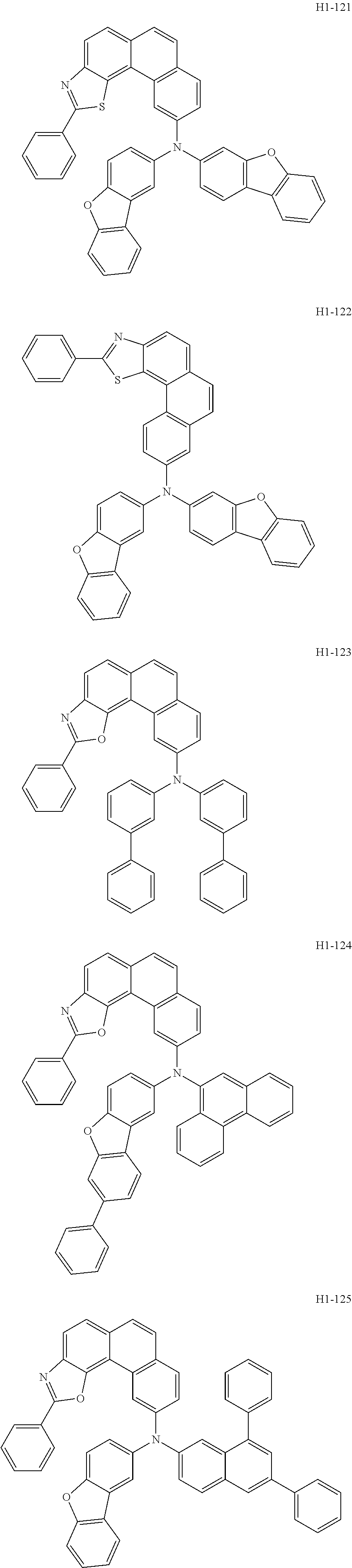 [SMILES: c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2nc3ccc4ccc5cc(N(c6ccc7c(c6)oc6ccccc67)c6ccc7oc8ccccc8c7c6)ccc5c4c3s2)cc1 ; c1ccc(-c2cccc(N(c3cccc(-c4ccccc4)c3)c3ccc4ccc5ccc6nc(-c7ccccc7)oc6c5c4c3)c2)cc1 ; c1ccc(-c2ccc3c(c2)oc2ccc(N(c4ccc5ccc6ccc7nc(-c8ccccc8)oc7c6c5c4)c4cc5ccccc5c5ccccc45)cc23)cc1 ; c1ccc(-c2cc(-c3ccccc3)c3cc(N(c4ccc5oc6ccccc6c5c4)c4ccc5ccc6ccc7nc(-c8ccccc8)oc7c6c5c4)ccc3c2)cc1]

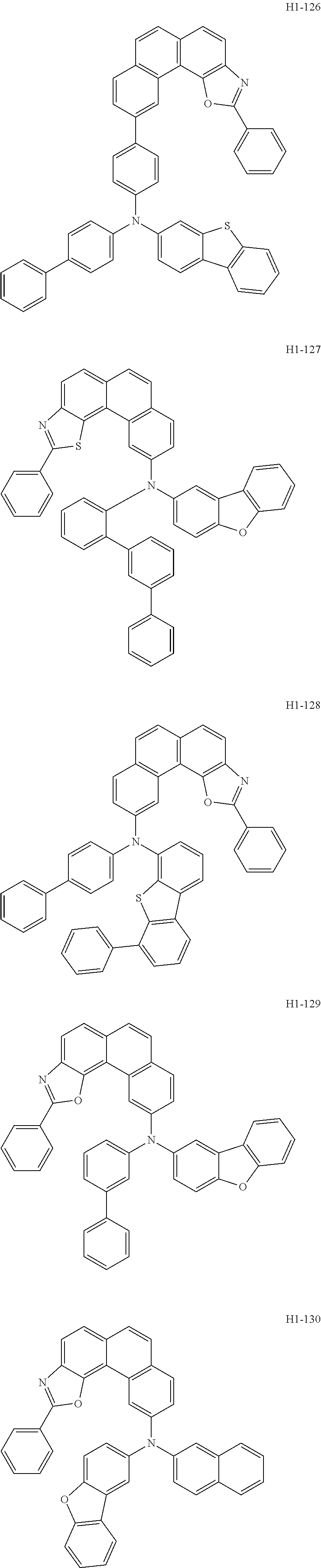 [SMILES: c1ccc(-c2ccc(N(c3ccc(-c4ccc5ccc6ccc7nc(-c8ccccc8)oc7c6c5c4)cc3)c3ccc4c(c3)sc3ccccc34)cc2)cc1 ; c1ccc(-c2cccc(-c3ccccc3N(c3ccc4oc5ccccc5c4c3)c3ccc4ccc5ccc6nc(-c7ccccc7)sc6c5c4c3)c2)cc1 ; c1ccc(-c2ccc(N(c3ccc4ccc5ccc6nc(-c7ccccc7)oc6c5c4c3)c3cccc4c3sc3c(-c5ccccc5)cccc34)cc2)cc1 ; c1ccc(-c2cccc(N(c3ccc4oc5ccccc5c4c3)c3ccc4ccc5ccc6nc(-c7ccccc7)oc6c5c4c3)c2)cc1 ; c1ccc(-c2nc3ccc4ccc5ccc(N(c6ccc7ccccc7c6)c6ccc7oc8ccccc8c7c6)cc5c4c3o2)cc1]

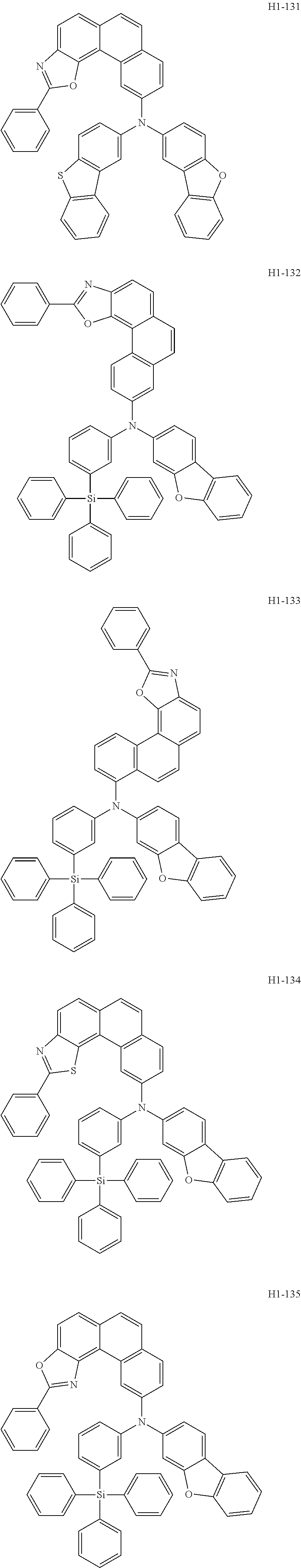

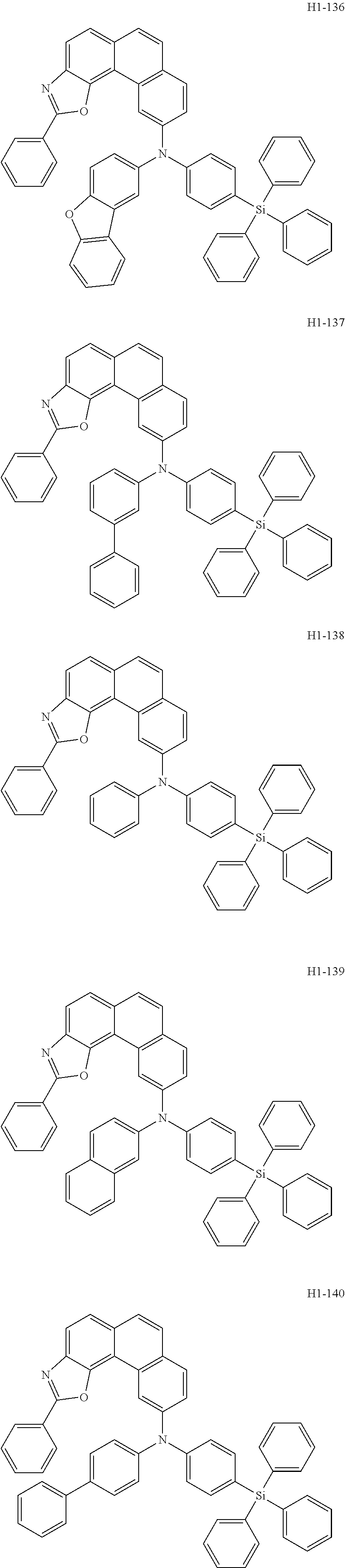

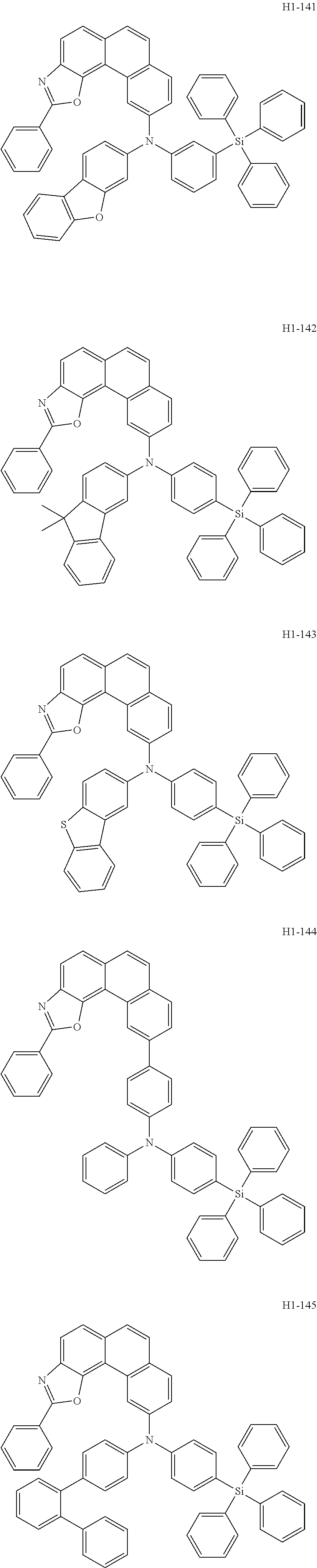

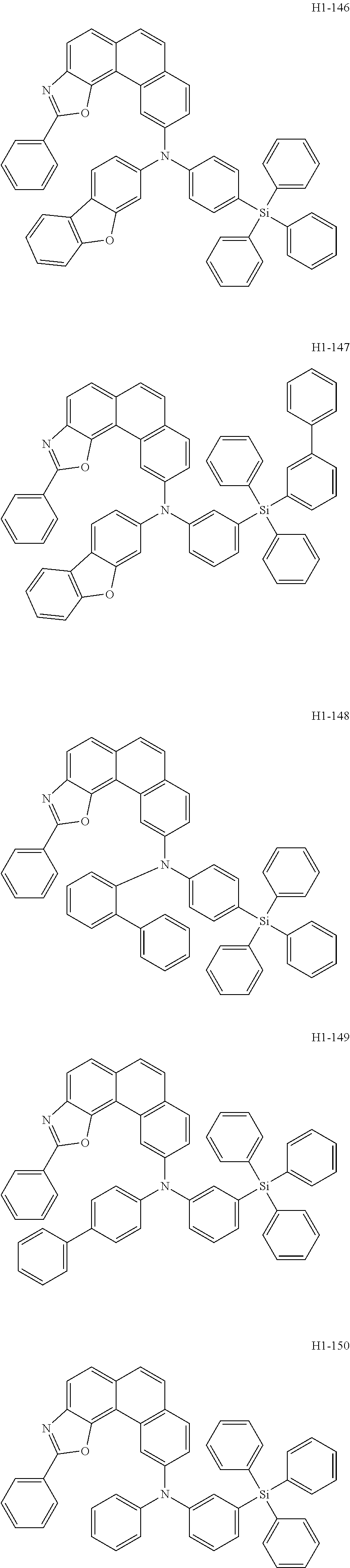

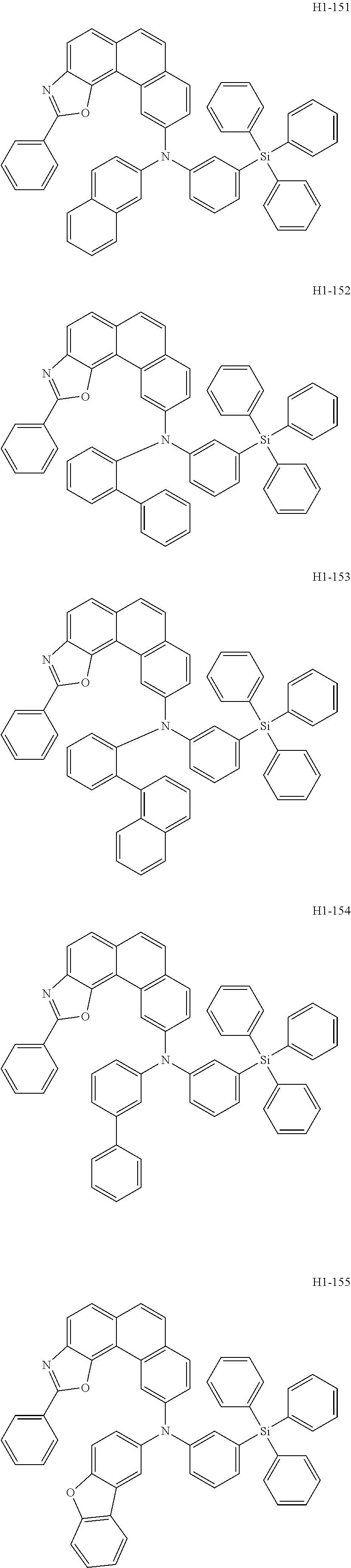

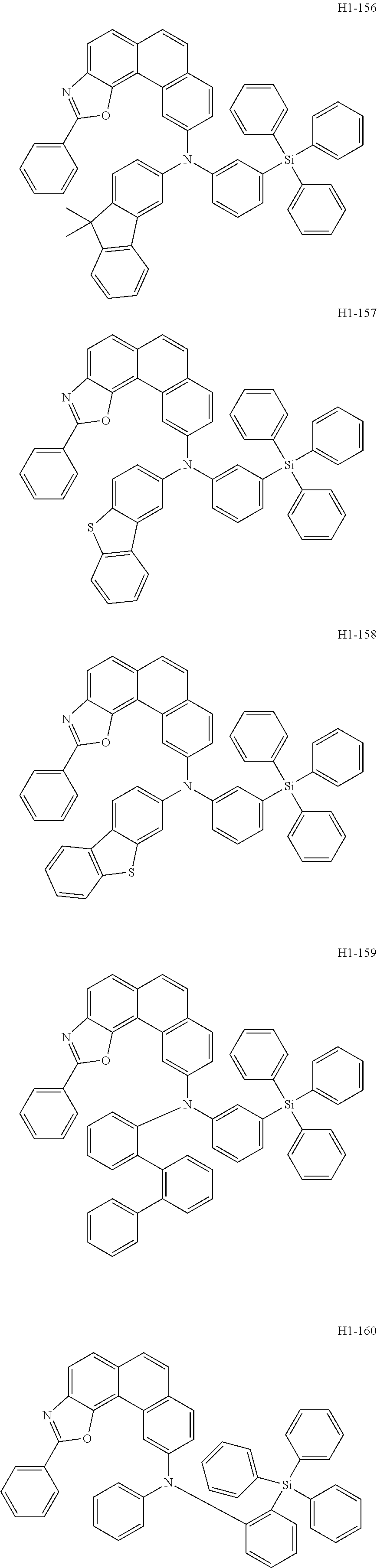

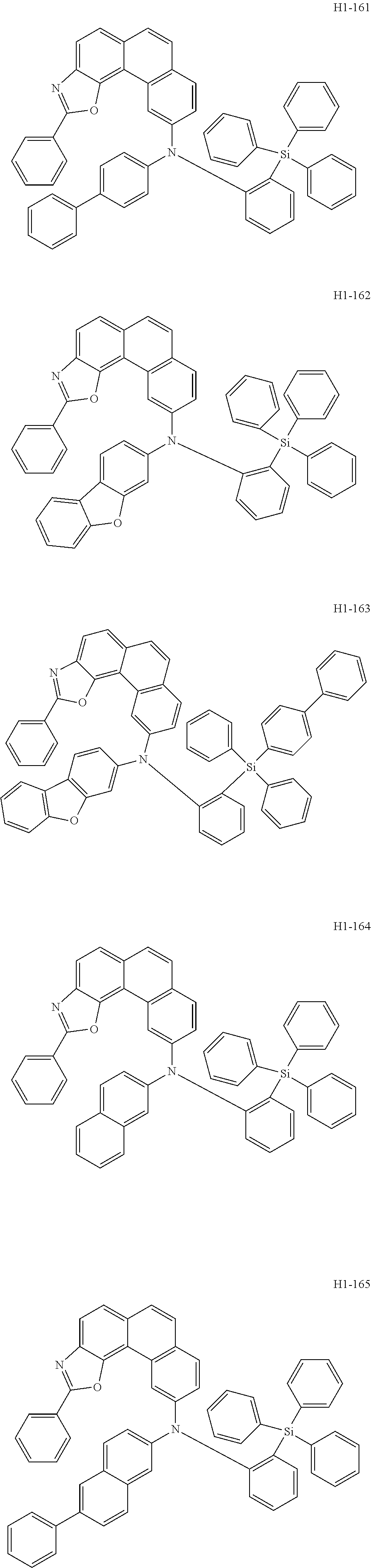

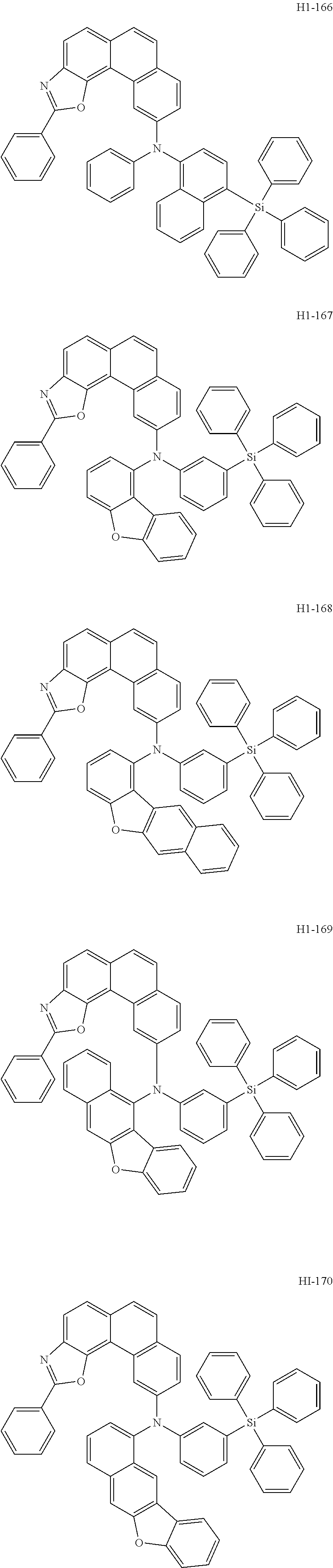

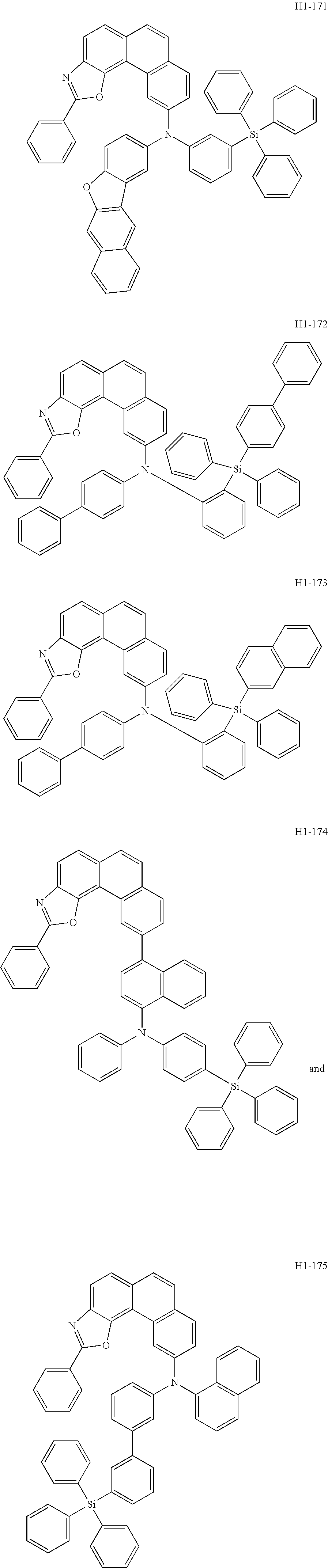

10. A plurality of host materials comprising a first host material and a second host material, wherein the first host material comprises an organic electroluminescent compound according to claim 1 and the second host material comprises an organic electroluminescent compound represented by the following formula 3: 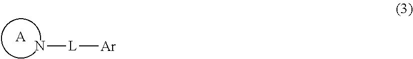
wherein, 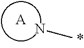
is a structure in which 5 to 8 substituted or unsubstituted (C6-C30)arene rings or a substituted or unsubstituted (3- to 30-membered)heteroarene ring are fused around a 7-membered or 8-membered ring, wherein, 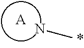
includes at least one pentacyclic ring(s) containing at least one nitrogen(s);
L represents a single bond, a substituted or unsubstituted (C1-C30)alkylene, a substituted or unsubstituted (C3-C30)cycloalkylene, a substituted or unsubstituted (C6-C30)arylene, or a substituted or unsubstituted (3- to 30-membered)heteroarylene;
Ar represents deuterium, halogen, cyano, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₁₁R₁₂, or —SiR₁₃R₁₄R₁₅; and
R₁₁ to R₁₅ each independently represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

11. The plurality of host materials according to claim 10, wherein 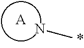
is represented by any one of the following formulas 3-1 to 3-3: 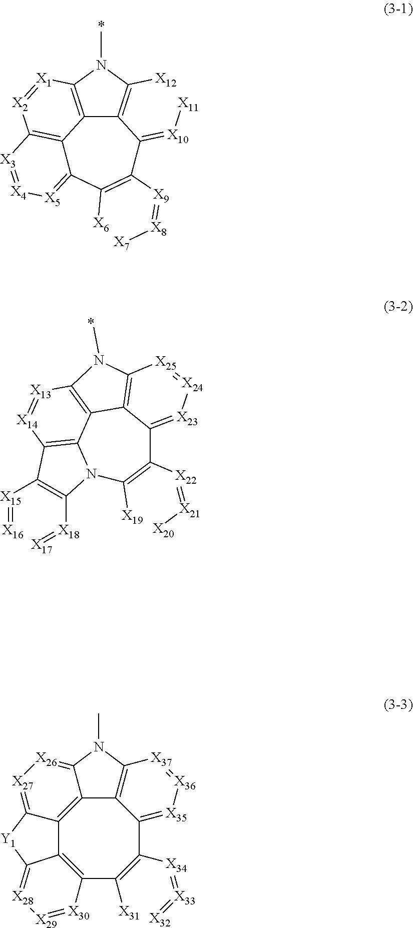
wherein,
X₁ to X₃₇ each independently represent —N═ or —C(R₁₁)═;
R₁₁ represents hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or adjacent Rₐs may be linked to each other to form a ring(s);
Y₁ represents —N(L₁₁-(Ar₁₁)ₘ)—, —O—, —S—, or —C(R₁₂)(R₁₃)—;
L₁₁ represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₁₁ represents a substituted or unsubstituted (C6-C30)aryl, a substituted or unsubstituted (3- to 30-membered)heteroaryl, or —N(R₁₄)(R₁₅);
R₁₂ to R₁₅ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, or a substituted or unsubstituted (C3-C30)cycloalkyl; or may be linked to each other to form a ring(s);
m is an integer of 1 or 2; and
when m is 2, each of Ar₁₁ may be the same or different.

12. The plurality of host materials according to claim 11, wherein the formula 3-1 is represented by the following formula 3-1-1: 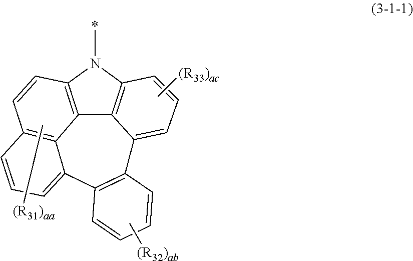
wherein,
R₃₁ to R₃₃ are each independently as defined as R₁₁ in claim 11;
aa is an integer of 1 to 5, ab is an integer of 1 to 4, and ac is an integer of 1 to 3; and
when aa, ab, and ac are 2 or more, each of R₃₁, each of R₃₂ and each of R₃₃ may be the same or different.

13. The plurality of host materials according to claim 11, wherein the formula 3-2 is represented by the following formula 3-2-1: 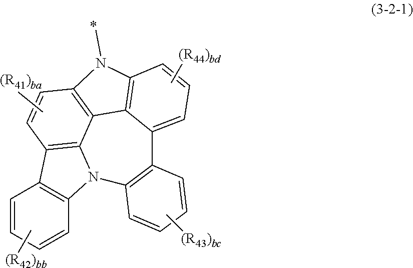
wherein,
R₄₁ to R₄₄ are each independently as defined as R₁₁ in claim 11;
ba is an integer of 1 or 2, bb and bc are each independently an integer of 1 to 4, and bd is an integer of 1 to 3; and
when ba, bb, bc, and bd are 2 or more, each of R₄₁, each of R₄₂, each of R₄₃ and each of R₄₄ may be the same or different.

14. The plurality of host materials according to claim 11, wherein the formula 3-3 is represented by the following formula 3-3-1: 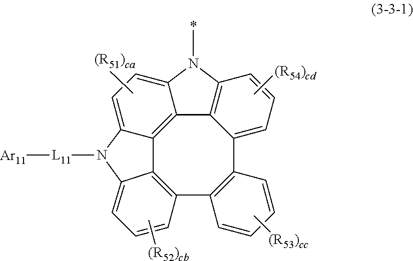
wherein,
R₅₁ to R₅₄ are each independently as defined as R₁₁ in claim 11;
L₁₁ and Ar₁₁ are as defined in claim 11;
ca is an integer of 1 or 2, cb and cd are each independently an integer of 1 to 3, and cc is an integer of 1 to 4; and
when ca, cb, cc, and cd are 2 or more, each of R₅₁, each of R₅₂, each of R₅₃ and each of R₅₄ may be the same or different.

15. The plurality of host materials according to claim 10, wherein the compound represented by formula 3 is selected from the following compounds:

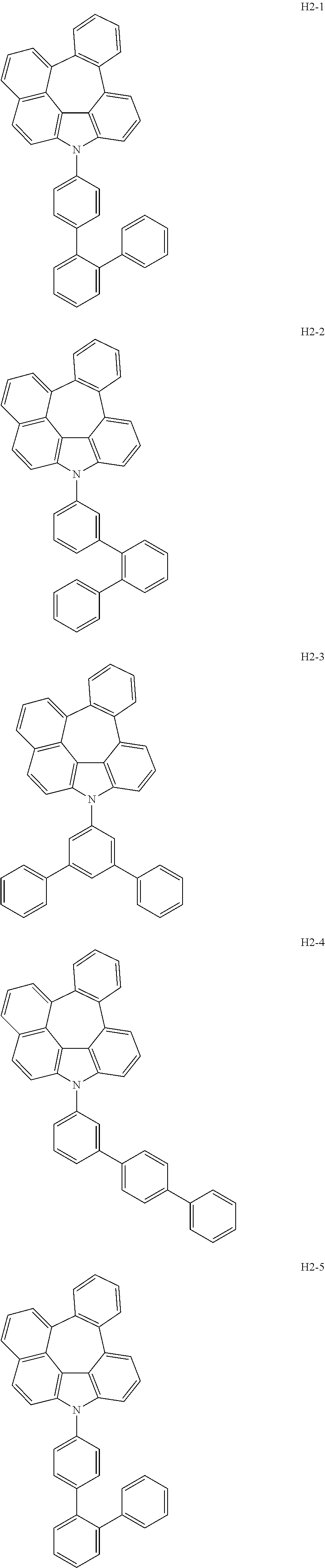 [SMILES: c1ccc(-c2ccccc2-c2ccc(-n3c4cccc5c4c4c6c(cccc6ccc43)-c3ccccc3-5)cc2)cc1 ; c1ccc(-c2ccccc2-c2cccc(-n3c4cccc5c4c4c6c(cccc6ccc43)-c3ccccc3-5)c2)cc1 ; c1ccc(-c2cc(-c3ccccc3)cc(-n3c4cccc5c4c4c6c(cccc6ccc43)-c3ccccc3-5)c2)cc1 ; c1ccc(-c2ccc(-c3cccc(-n4c5cccc6c5c5c7c(cccc7ccc54)-c4ccccc4-6)c3)cc2)cc1 ; c1ccc(-c2ccccc2-c2ccc(-n3c4cccc5c4c4c6c(cccc6ccc43)-c3ccccc3-5)cc2)cc1]

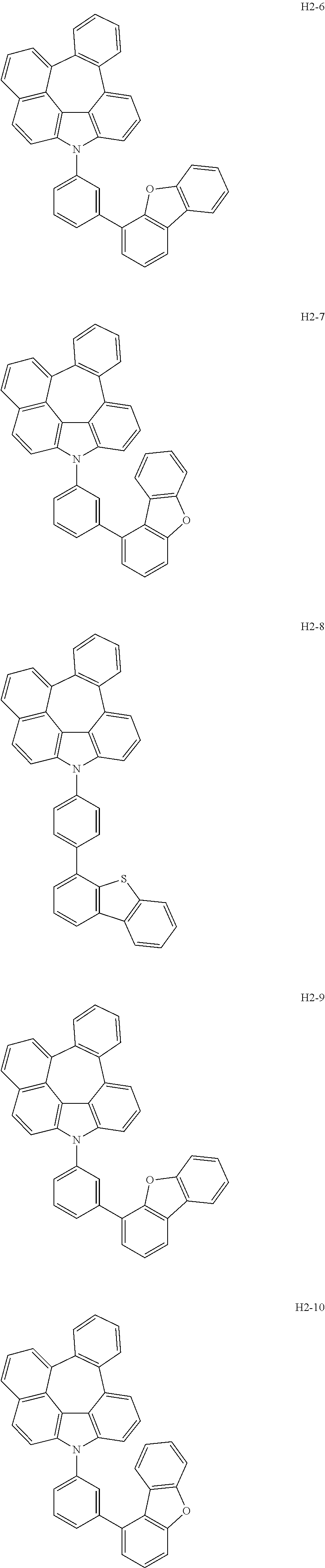 [SMILES: c1cc(-c2cccc3c2oc2ccccc23)cc(-n2c3cccc4c3c3c5c(cccc5ccc32)-c2ccccc2-4)c1 ; c1cc(-c2cccc3oc4ccccc4c23)cc(-n2c3cccc4c3c3c5c(cccc5ccc32)-c2ccccc2-4)c1 ; c1ccc2c(c1)-c1cccc3ccc4c(c13)c1c-2cccc1n4-c1ccc(-c2cccc3c2sc2ccccc23)cc1 ; c1cc(-c2cccc3c2oc2ccccc23)cc(-n2c3cccc4c3c3c5c(cccc5ccc32)-c2ccccc2-4)c1 ; c1cc(-c2cccc3oc4ccccc4c23)cc(-n2c3cccc4c3c3c5c(cccc5ccc32)-c2ccccc2-4)c1]

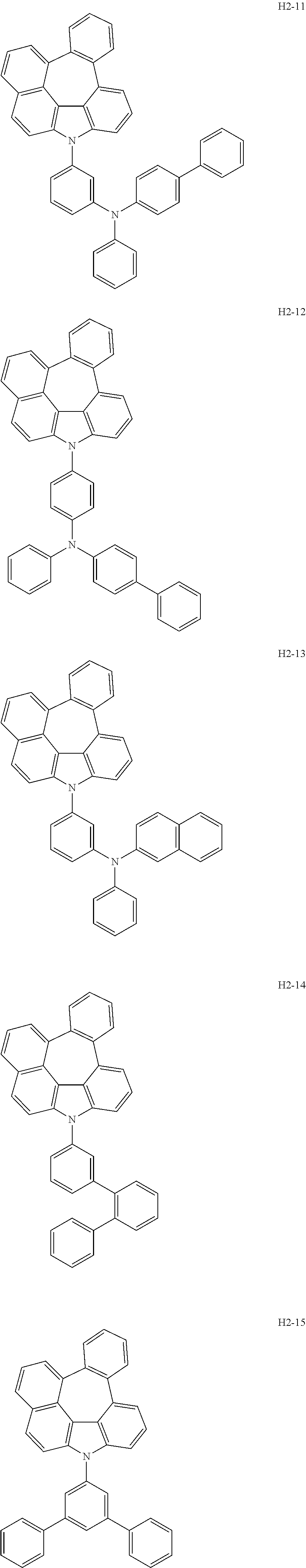 [SMILES: c1ccc(-c2ccc(N(c3ccccc3)c3cccc(-n4c5cccc6c5c5c7c(cccc7ccc54)-c4ccccc4-6)c3)cc2)cc1 ; c1ccc(-c2ccc(N(c3ccccc3)c3ccc(-n4c5cccc6c5c5c7c(cccc7ccc54)-c4ccccc4-6)cc3)cc2)cc1 ; c1ccc(N(c2cccc(-n3c4cccc5c4c4c6c(cccc6ccc43)-c3ccccc3-5)c2)c2ccc3ccccc3c2)cc1 ; c1ccc(-c2ccccc2-c2cccc(-n3c4cccc5c4c4c6c(cccc6ccc43)-c3ccccc3-5)c2)cc1 ; c1ccc(-c2cc(-c3ccccc3)cc(-n3c4cccc5c4c4c6c(cccc6ccc43)-c3ccccc3-5)c2)cc1]

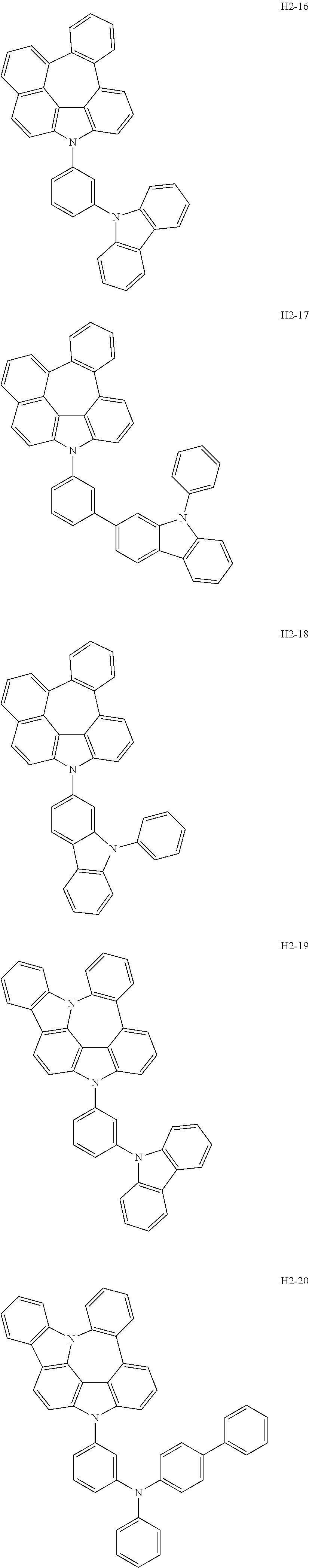 [SMILES: c1cc(-n2c3ccccc3c3ccccc32)cc(-n2c3cccc4c3c3c5c(cccc5ccc32)-c2ccccc2-4)c1 ; c1ccc(-n2c3ccccc3c3ccc(-c4cccc(-n5c6cccc7c6c6c8c(cccc8ccc65)-c5ccccc5-7)c4)cc32)cc1 ; c1ccc(-n2c3ccccc3c3ccc(-n4c5cccc6c5c5c7c(cccc7ccc54)-c4ccccc4-6)cc32)cc1 ; c1cc(-n2c3ccccc3c3ccccc32)cc(-n2c3cccc4c5ccccc5n5c6ccccc6c6ccc2c(c43)c65)c1 ; c1ccc(-c2ccc(N(c3ccccc3)c3cccc(-n4c5cccc6c7ccccc7n7c8ccccc8c8ccc4c(c65)c87)c3)cc2)cc1]

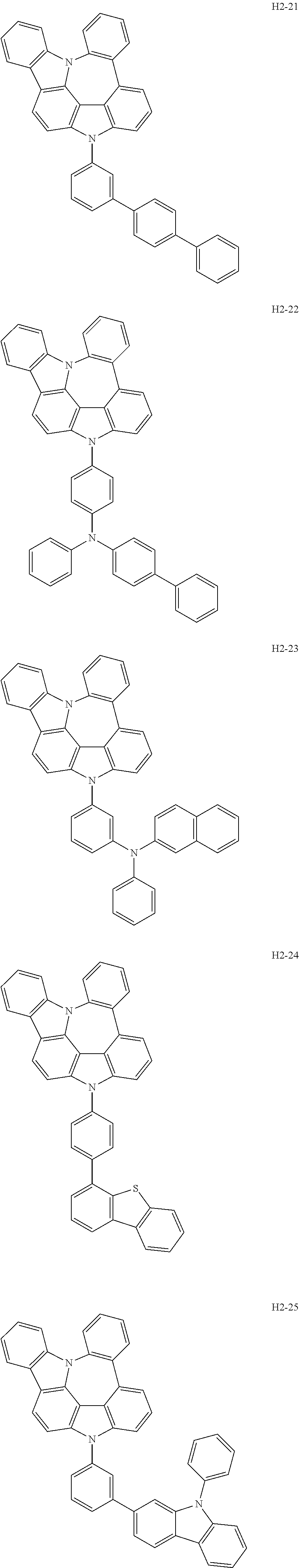 [SMILES: c1ccc(-c2ccc(-c3cccc(-n4c5cccc6c7ccccc7n7c8ccccc8c8ccc4c(c65)c87)c3)cc2)cc1 ; c1ccc(-c2ccc(N(c3ccccc3)c3ccc(-n4c5cccc6c7ccccc7n7c8ccccc8c8ccc4c(c65)c87)cc3)cc2)cc1 ; c1ccc(N(c2cccc(-n3c4cccc5c6ccccc6n6c7ccccc7c7ccc3c(c54)c76)c2)c2ccc3ccccc3c2)cc1 ; c1ccc2c(c1)sc1c(-c3ccc(-n4c5cccc6c7ccccc7n7c8ccccc8c8ccc4c(c65)c87)cc3)cccc12 ; c1ccc(-n2c3ccccc3c3ccc(-c4cccc(-n5c6cccc7c8ccccc8n8c9ccccc9c9ccc5c(c76)c98)c4)cc32)cc1]

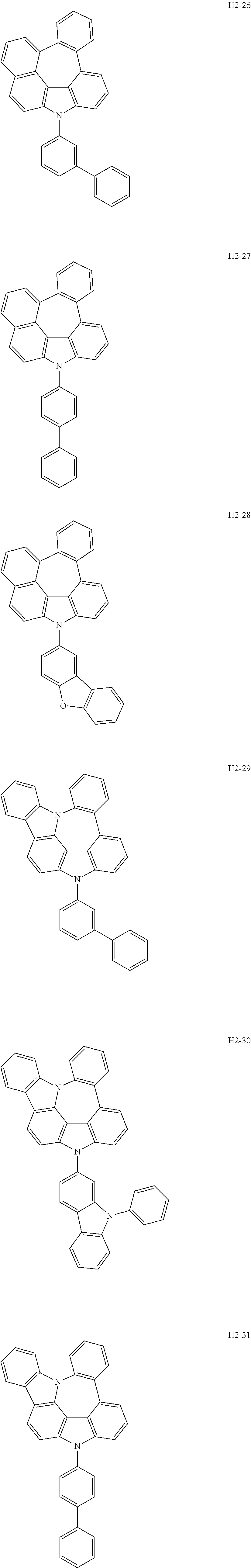 [SMILES: c1ccc(-c2cccc(-n3c4cccc5c4c4c6c(cccc6ccc43)-c3ccccc3-5)c2)cc1 ; c1ccc(-c2ccc(-n3c4cccc5c4c4c6c(cccc6ccc43)-c3ccccc3-5)cc2)cc1 ; c1ccc2c(c1)-c1cccc3ccc4c(c13)c1c-2cccc1n4-c1ccc2oc3ccccc3c2c1 ; c1ccc(-c2cccc(-n3c4cccc5c6ccccc6n6c7ccccc7c7ccc3c(c54)c76)c2)cc1 ; c1ccc(-n2c3ccccc3c3ccc(-n4c5cccc6c7ccccc7n7c8ccccc8c8ccc4c(c65)c87)cc32)cc1 ; c1ccc(-c2ccc(-n3c4cccc5c6ccccc6n6c7ccccc7c7ccc3c(c54)c76)cc2)cc1]

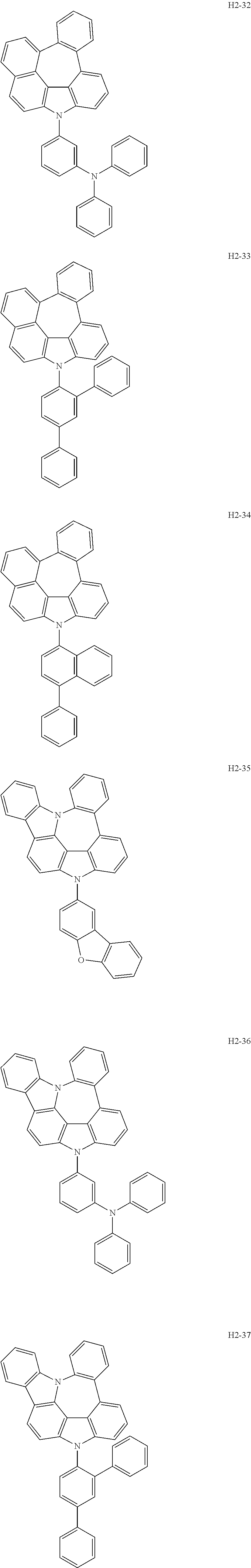 [SMILES: c1ccc(N(c2ccccc2)c2cccc(-n3c4cccc5c4c4c6c(cccc6ccc43)-c3ccccc3-5)c2)cc1 ; c1ccc(-c2ccc(-n3c4cccc5c4c4c6c(cccc6ccc43)-c3ccccc3-5)c(-c3ccccc3)c2)cc1 ; c1ccc(-c2ccc(-n3c4cccc5c4c4c6c(cccc6ccc43)-c3ccccc3-5)c3ccccc23)cc1 ; c1ccc2c(c1)oc1ccc(-n3c4cccc5c6ccccc6n6c7ccccc7c7ccc3c(c54)c76)cc12 ; c1ccc(N(c2ccccc2)c2cccc(-n3c4cccc5c6ccccc6n6c7ccccc7c7ccc3c(c54)c76)c2)cc1 ; c1ccc(-c2ccc(-n3c4cccc5c6ccccc6n6c7ccccc7c7ccc3c(c54)c76)c(-c3ccccc3)c2)cc1]

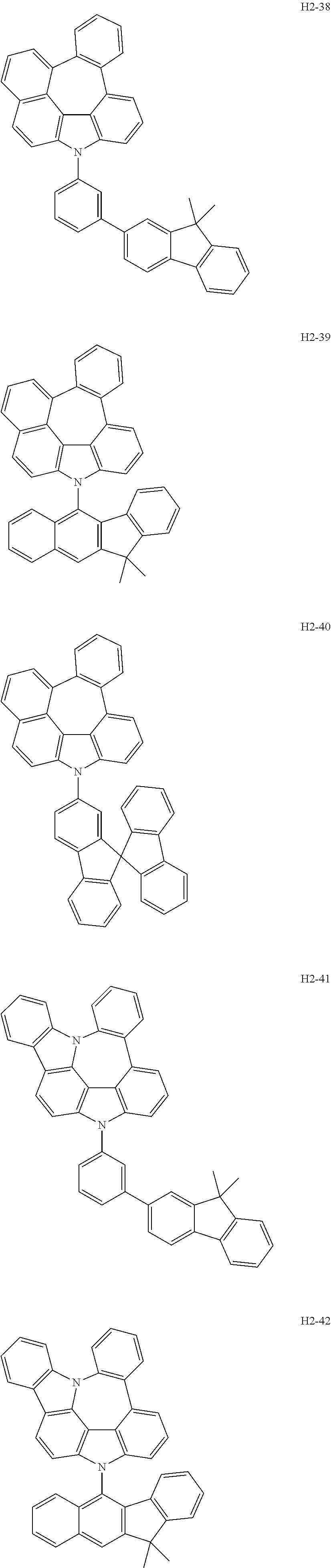 [SMILES: CC1(C)c2ccccc2-c2ccc(-c3cccc(-n4c5cccc6c5c5c7c(cccc7ccc54)-c4ccccc4-6)c3)cc21 ; CC1(C)c2ccccc2-c2c1cc1ccccc1c2-n1c2cccc3c2c2c4c(cccc4ccc21)-c1ccccc1-3 ; c1ccc2c(c1)-c1cccc3ccc4c(c13)c1c-2cccc1n4-c1ccc2c(c1)C1(c3ccccc3-c3ccccc31)c1ccccc1-2 ; CC1(C)c2ccccc2-c2ccc(-c3cccc(-n4c5cccc6c7ccccc7n7c8ccccc8c8ccc4c(c65)c87)c3)cc21 ; CC1(C)c2ccccc2-c2c1cc1ccccc1c2-n1c2cccc3c4ccccc4n4c5ccccc5c5ccc1c(c32)c54]

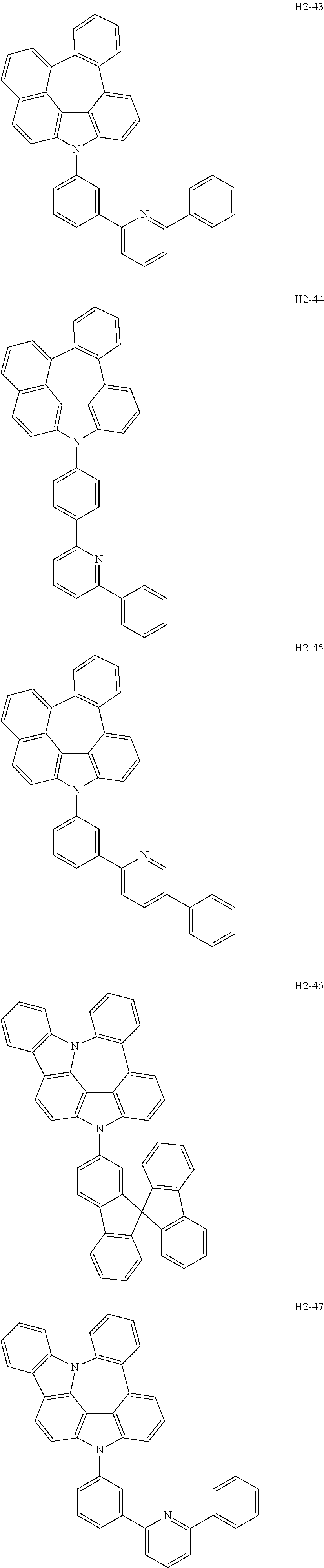 [SMILES: c1ccc(-c2cccc(-c3cccc(-n4c5cccc6c5c5c7c(cccc7ccc54)-c4ccccc4-6)c3)n2)cc1 ; c1ccc(-c2cccc(-c3ccc(-n4c5cccc6c5c5c7c(cccc7ccc54)-c4ccccc4-6)cc3)n2)cc1 ; c1ccc(-c2ccc(-c3cccc(-n4c5cccc6c5c5c7c(cccc7ccc54)-c4ccccc4-6)c3)nc2)cc1 ; c1ccc2c(c1)-c1ccccc1C21c2ccccc2-c2ccc(-n3c4cccc5c6ccccc6n6c7ccccc7c7ccc3c(c54)c76)cc21 ; c1ccc(-c2cccc(-c3cccc(-n4c5cccc6c7ccccc7n7c8ccccc8c8ccc4c(c65)c87)c3)n2)cc1]

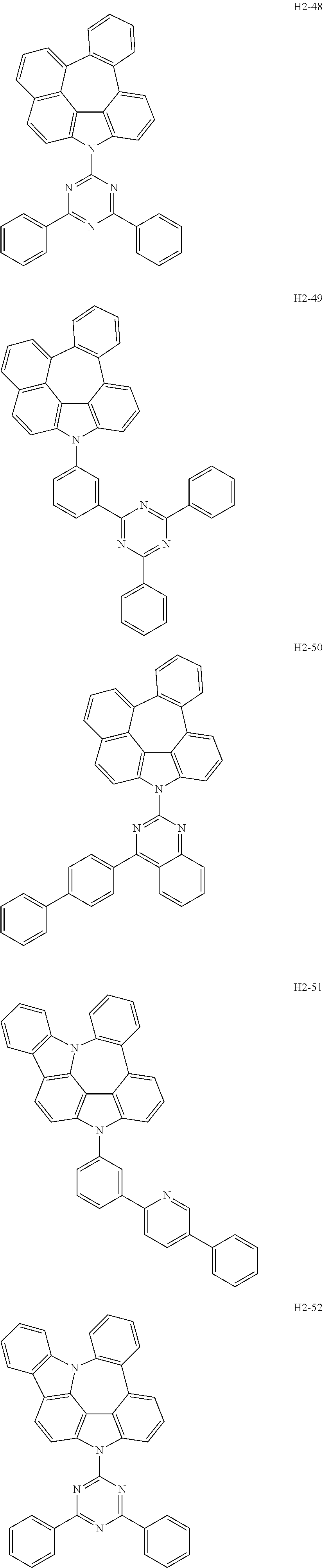

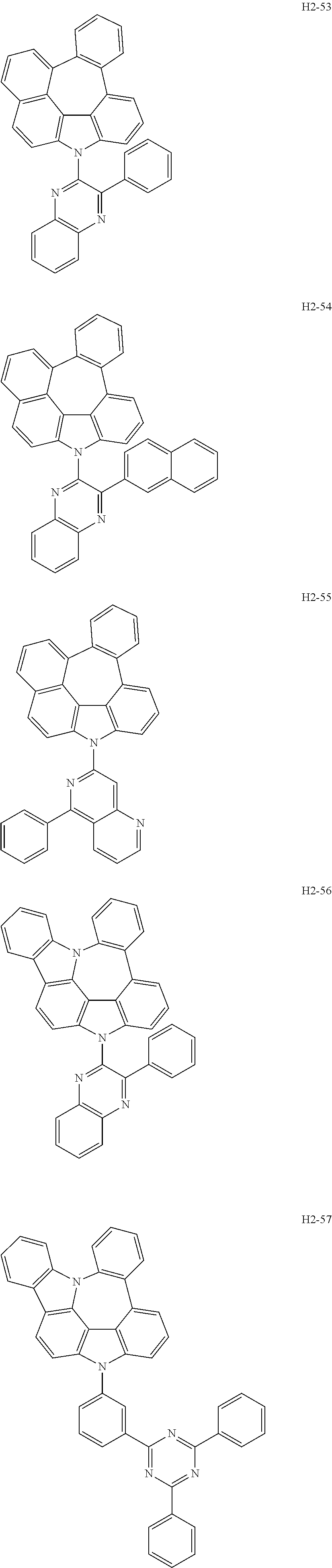

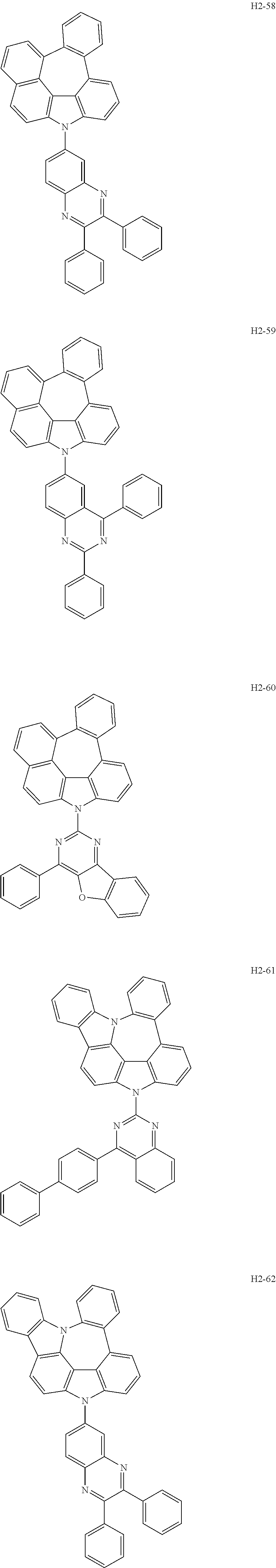 [SMILES: c1ccc(-c2nc3ccc(-n4c5cccc6c5c5c7c(cccc7ccc54)-c4ccccc4-6)cc3nc2-c2ccccc2)cc1 ; c1ccc(-c2nc(-c3ccccc3)c3cc(-n4c5cccc6c5c5c7c(cccc7ccc54)-c4ccccc4-6)ccc3n2)cc1 ; c1ccc(-c2nc(-n3c4cccc5c4c4c6c(cccc6ccc43)-c3ccccc3-5)nc3c2oc2ccccc23)cc1 ; c1ccc(-c2ccc(-c3nc(-n4c5cccc6c7ccccc7n7c8ccccc8c8ccc4c(c65)c87)nc4ccccc34)cc2)cc1 ; c1ccc(-c2nc3ccc(-n4c5cccc6c7ccccc7n7c8ccccc8c8ccc4c(c65)c87)cc3nc2-c2ccccc2)cc1]

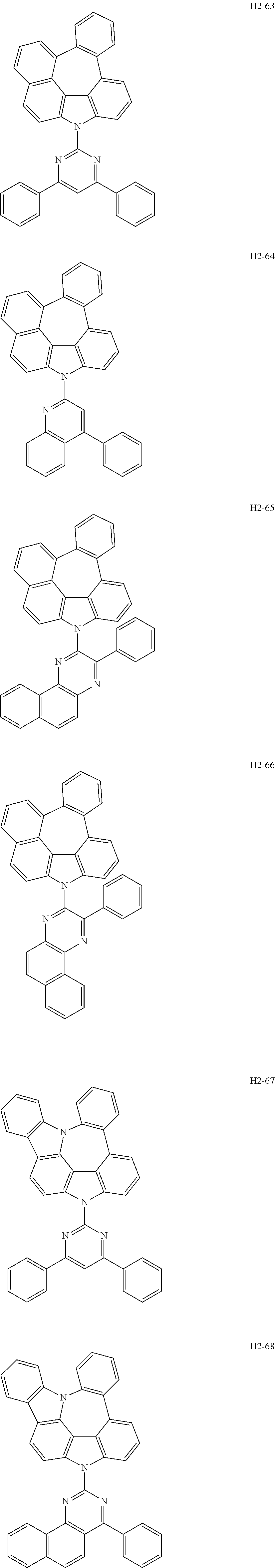 [SMILES: c1ccc(-c2cc(-c3ccccc3)nc(-n3c4cccc5c4c4c6c(cccc6ccc43)-c3ccccc3-5)n2)cc1 ; c1ccc(-c2cc(-n3c4cccc5c4c4c6c(cccc6ccc43)-c3ccccc3-5)nc3ccccc23)cc1 ; c1ccc(-c2nc3ccc4ccccc4c3nc2-n2c3cccc4c3c3c5c(cccc5ccc32)-c2ccccc2-4)cc1 ; c1ccc(-c2nc3c(ccc4ccccc43)nc2-n2c3cccc4c3c3c5c(cccc5ccc32)-c2ccccc2-4)cc1 ; c1ccc(-c2cc(-c3ccccc3)nc(-n3c4cccc5c6ccccc6n6c7ccccc7c7ccc3c(c54)c76)n2)cc1 ; c1ccc(-c2nc(-n3c4cccc5c6ccccc6n6c7ccccc7c7ccc3c(c54)c76)nc3c2ccc2ccccc23)cc1]

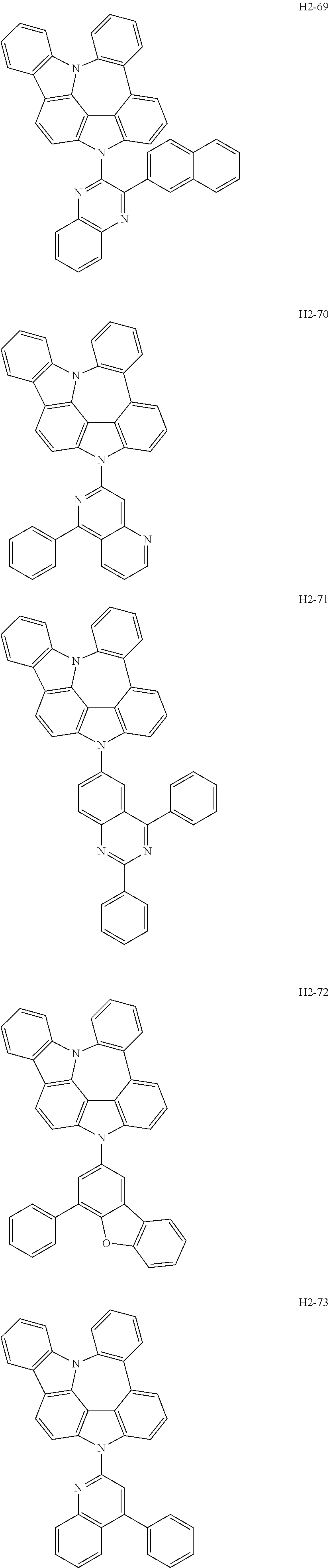

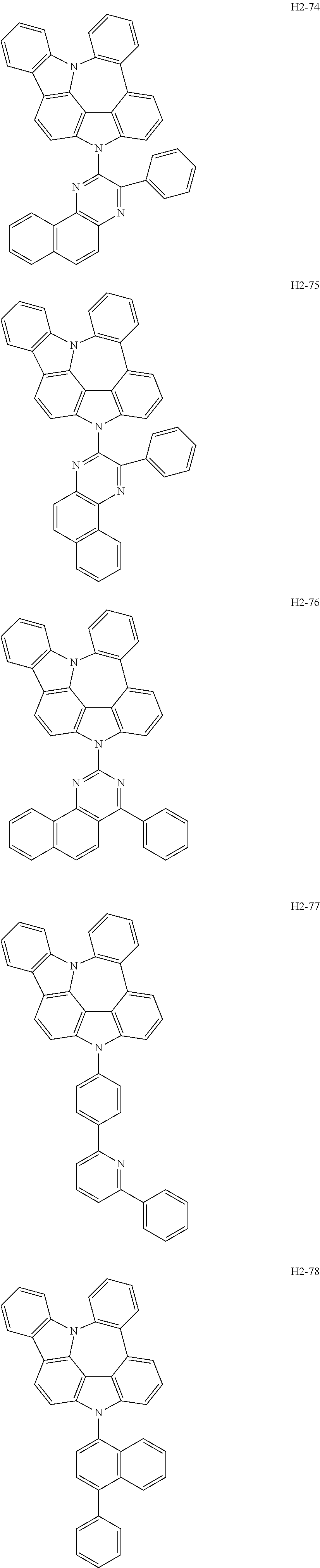

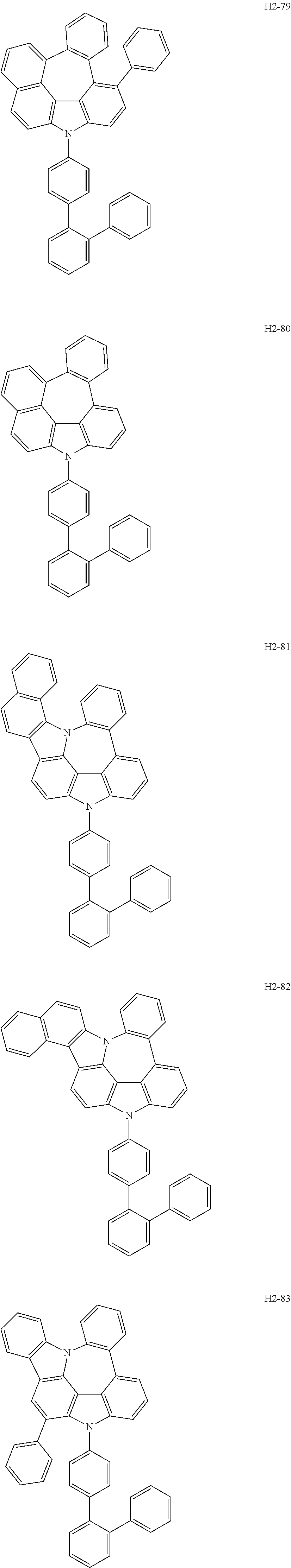 [SMILES: c1ccc(-c2ccccc2-c2ccc(-n3c4ccc(-c5ccccc5)c5c4c4c6c(cccc6ccc43)-c3ccccc3-5)cc2)cc1 ; c1ccc(-c2ccccc2-c2ccc(-n3c4cccc5c4c4c6c(cccc6ccc43)-c3ccccc3-5)cc2)cc1 ; c1ccc(-c2ccccc2-c2ccc(-n3c4cccc5c6ccccc6n6c7c8ccccc8ccc7c7ccc3c(c54)c76)cc2)cc1 ; c1ccc(-c2ccccc2-c2ccc(-n3c4cccc5c6ccccc6n6c7ccc8ccccc8c7c7ccc3c(c54)c76)cc2)cc1 ; c1ccc(-c2ccccc2-c2ccc(-n3c4cccc5c6ccccc6n6c7ccccc7c7cc(-c8ccccc8)c3c(c54)c76)cc2)cc1]

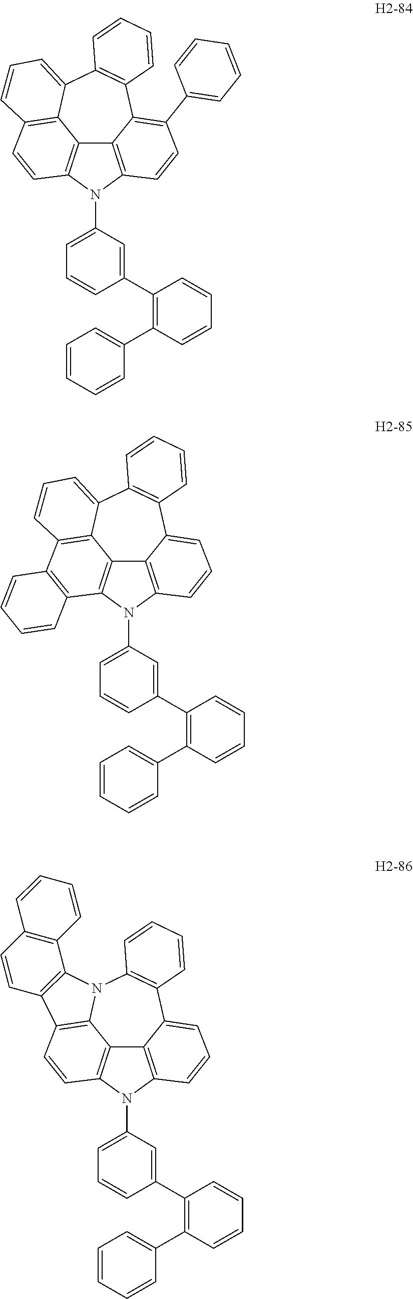 [SMILES: c1ccc(-c2ccccc2-c2cccc(-n3c4ccc(-c5ccccc5)c5c4c4c6c(cccc6ccc43)-c3ccccc3-5)c2)cc1 ; c1ccc(-c2ccccc2-c2cccc(-n3c4cccc5c4c4c6c(cccc6c6ccccc6c43)-c3ccccc3-5)c2)cc1 ; c1ccc(-c2ccccc2-c2cccc(-n3c4cccc5c6ccccc6n6c7c8ccccc8ccc7c7ccc3c(c54)c76)c2)cc1]

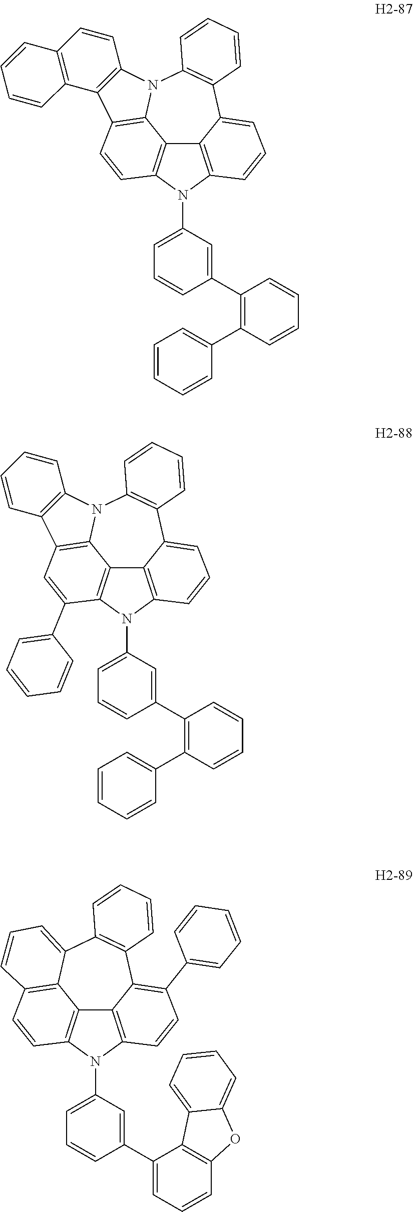 [SMILES: c1ccc(-c2ccccc2-c2cccc(-n3c4cccc5c6ccccc6n6c7ccc8ccccc8c7c7ccc3c(c54)c76)c2)cc1 ; c1ccc(-c2ccccc2-c2cccc(-n3c4cccc5c6ccccc6n6c7ccccc7c7cc(-c8ccccc8)c3c(c54)c76)c2)cc1 ; c1ccc(-c2ccc3c4c2-c2ccccc2-c2cccc5ccc(c4c25)n3-c2cccc(-c3cccc4oc5ccccc5c34)c2)cc1]

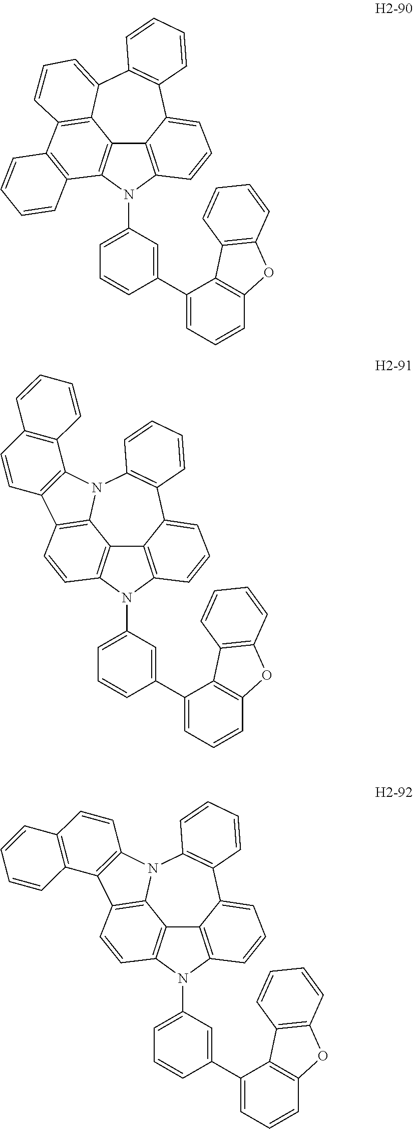 [SMILES: c1cc(-c2cccc3oc4ccccc4c23)cc(-n2c3cccc4c3c3c5c(cccc5c5ccccc5c32)-c2ccccc2-4)c1 ; c1cc(-c2cccc3oc4ccccc4c23)cc(-n2c3cccc4c5ccccc5n5c6c7ccccc7ccc6c6ccc2c(c43)c65)c1 ; c1cc(-c2cccc3oc4ccccc4c23)cc(-n2c3cccc4c5ccccc5n5c6ccc7ccccc7c6c6ccc2c(c43)c65)c1]

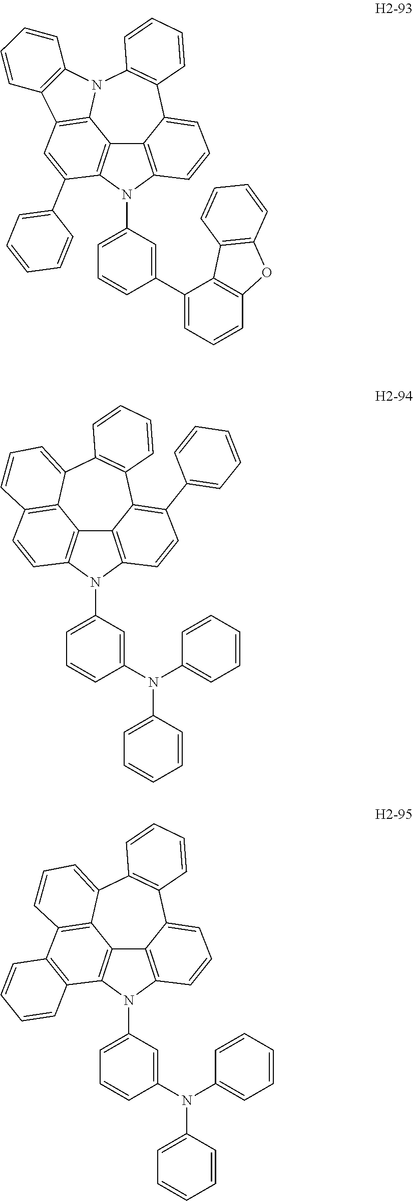

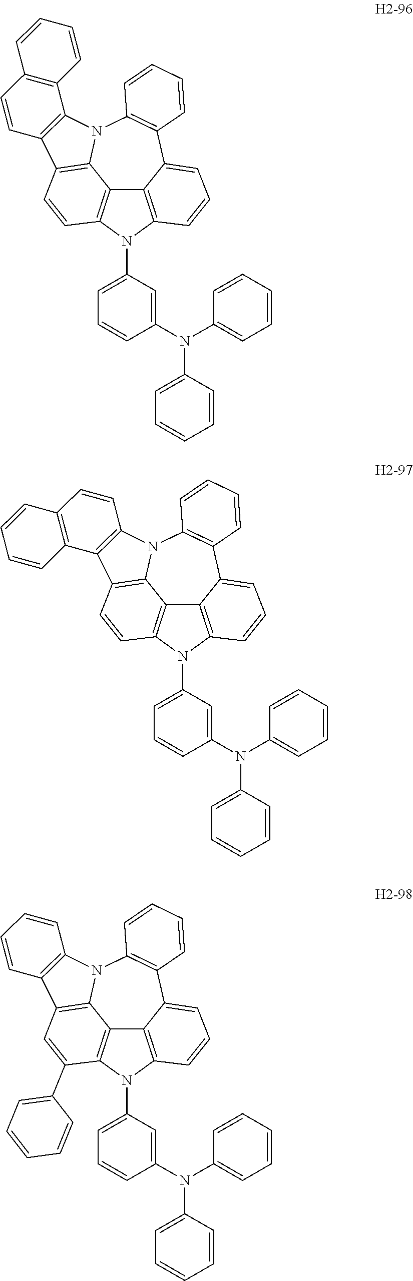

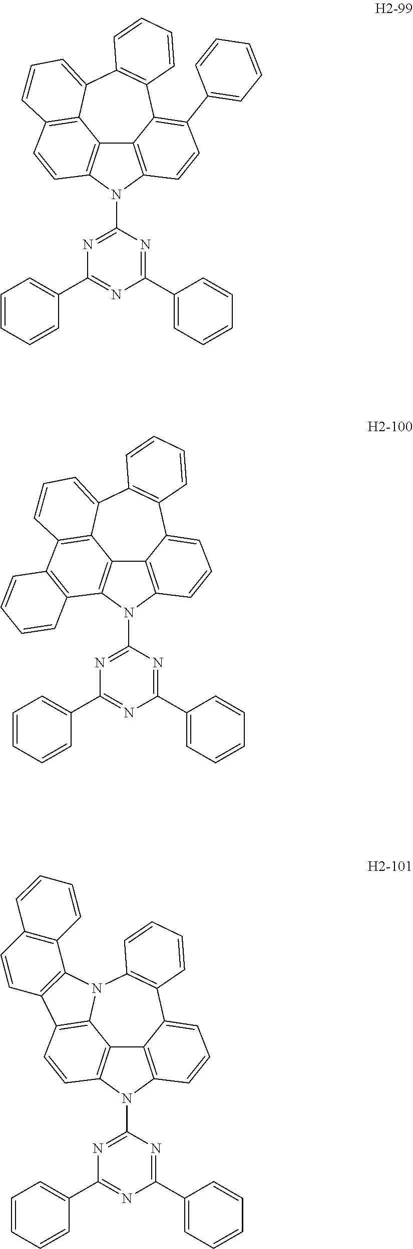 [SMILES: c1ccc(-c2nc(-c3ccccc3)nc(-n3c4ccc(-c5ccccc5)c5c4c4c6c(cccc6ccc43)-c3ccccc3-5)n2)cc1 ; c1ccc(-c2nc(-c3ccccc3)nc(-n3c4cccc5c4c4c6c(cccc6c6ccccc6c43)-c3ccccc3-5)n2)cc1 ; c1ccc(-c2nc(-c3ccccc3)nc(-n3c4cccc5c6ccccc6n6c7c8ccccc8ccc7c7ccc3c(c54)c76)n2)cc1]

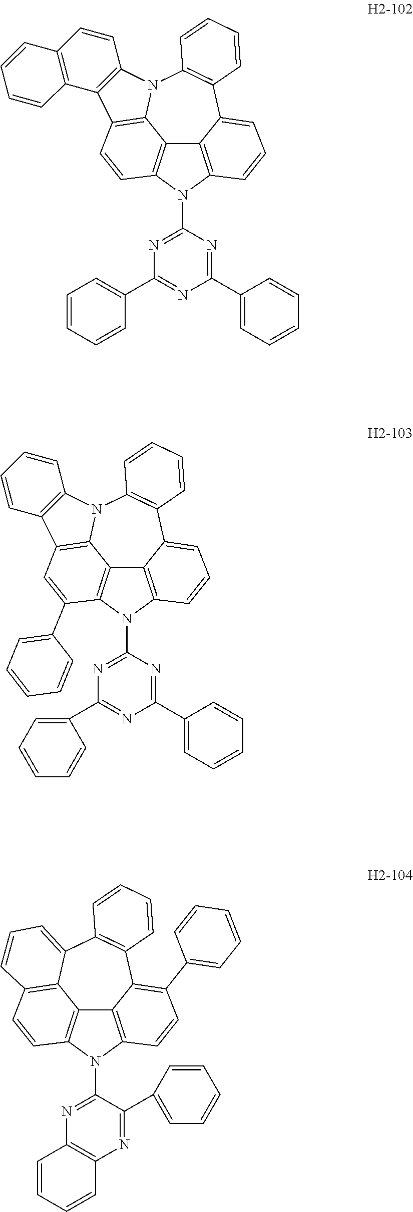 [SMILES: c1ccc(-c2nc(-c3ccccc3)nc(-n3c4cccc5c6ccccc6n6c7ccc8ccccc8c7c7ccc3c(c54)c76)n2)cc1 ; c1ccc(-c2nc(-c3ccccc3)nc(-n3c4cccc5c6ccccc6n6c7ccccc7c7cc(-c8ccccc8)c3c(c54)c76)n2)cc1 ; c1ccc(-c2ccc3c4c2-c2ccccc2-c2cccc5ccc(c4c25)n3-c2nc3ccccc3nc2-c2ccccc2)cc1]

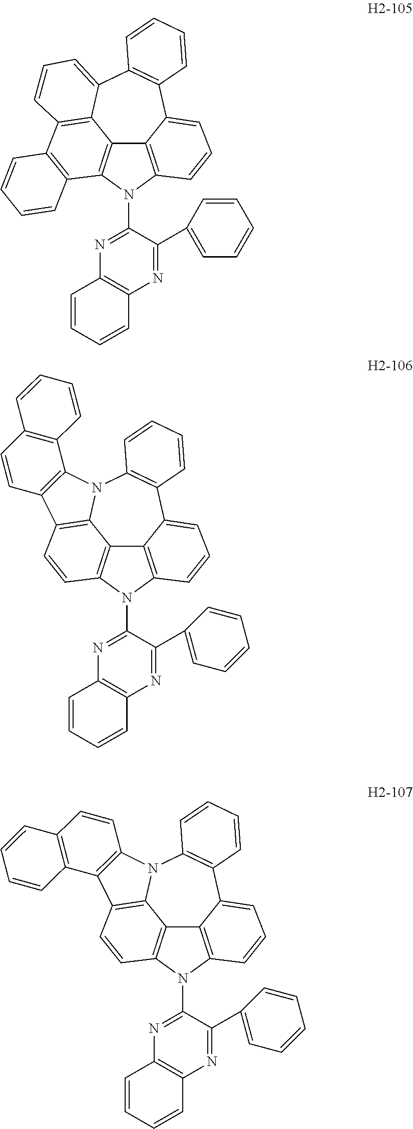 [SMILES: c1ccc(-c2nc3ccccc3nc2-n2c3cccc4c3c3c5c(cccc5c5ccccc5c32)-c2ccccc2-4)cc1 ; c1ccc(-c2nc3ccccc3nc2-n2c3cccc4c5ccccc5n5c6c7ccccc7ccc6c6ccc2c(c43)c65)cc1 ; c1ccc(-c2nc3ccccc3nc2-n2c3cccc4c5ccccc5n5c6ccc7ccccc7c6c6ccc2c(c43)c65)cc1]

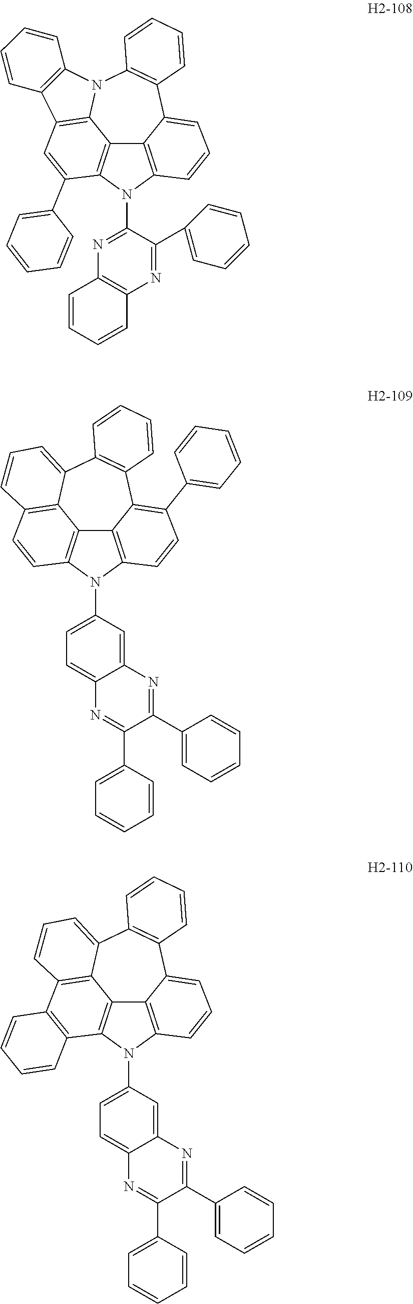 [SMILES: c1ccc(-c2nc3ccccc3nc2-n2c3cccc4c5ccccc5n5c6ccccc6c6cc(-c7ccccc7)c2c(c43)c65)cc1 ; c1ccc(-c2ccc3c4c2-c2ccccc2-c2cccc5ccc(c4c25)n3-c2ccc3nc(-c4ccccc4)c(-c4ccccc4)nc3c2)cc1 ; c1ccc(-c2nc3ccc(-n4c5cccc6c5c5c7c(cccc7c7ccccc7c54)-c4ccccc4-6)cc3nc2-c2ccccc2)cc1]

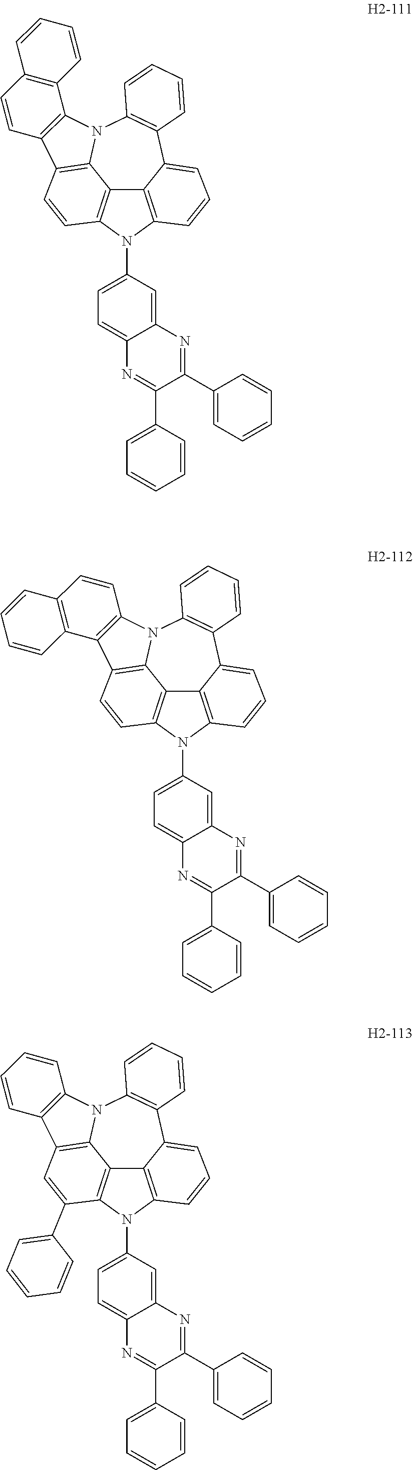 [SMILES: c1ccc(-c2nc3ccc(-n4c5cccc6c7ccccc7n7c8c9ccccc9ccc8c8ccc4c(c65)c87)cc3nc2-c2ccccc2)cc1 ; c1ccc(-c2nc3ccc(-n4c5cccc6c7ccccc7n7c8ccc9ccccc9c8c8ccc4c(c65)c87)cc3nc2-c2ccccc2)cc1 ; c1ccc(-c2nc3ccc(-n4c5cccc6c7ccccc7n7c8ccccc8c8cc(-c9ccccc9)c4c(c65)c87)cc3nc2-c2ccccc2)cc1]

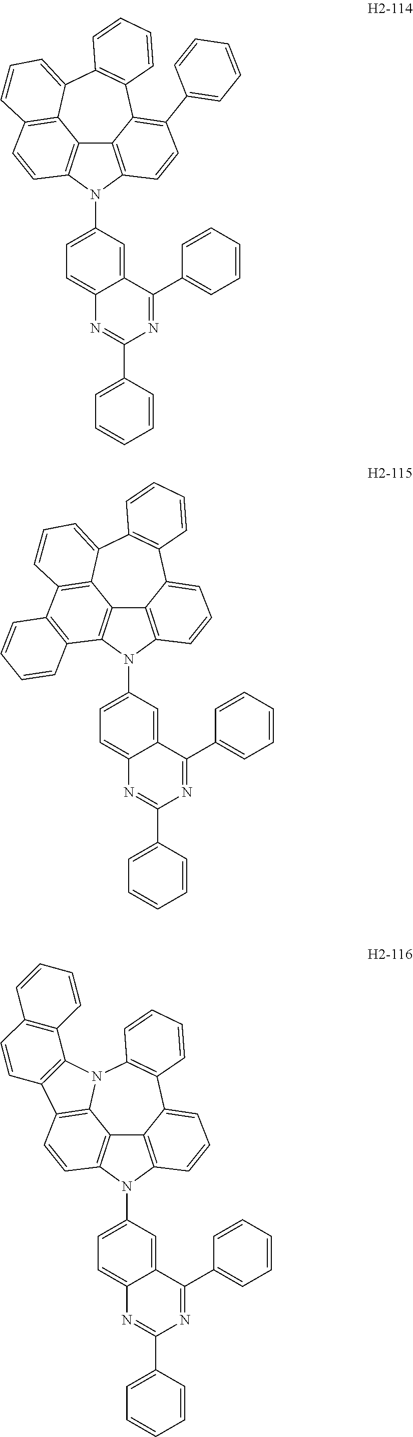 [SMILES: c1ccc(-c2nc(-c3ccccc3)c3cc(-n4c5ccc(-c6ccccc6)c6c5c5c7c(cccc7ccc54)-c4ccccc4-6)ccc3n2)cc1 ; c1ccc(-c2nc(-c3ccccc3)c3cc(-n4c5cccc6c5c5c7c(cccc7c7ccccc7c54)-c4ccccc4-6)ccc3n2)cc1 ; c1ccc(-c2nc(-c3ccccc3)c3cc(-n4c5cccc6c7ccccc7n7c8c9ccccc9ccc8c8ccc4c(c65)c87)ccc3n2)cc1]

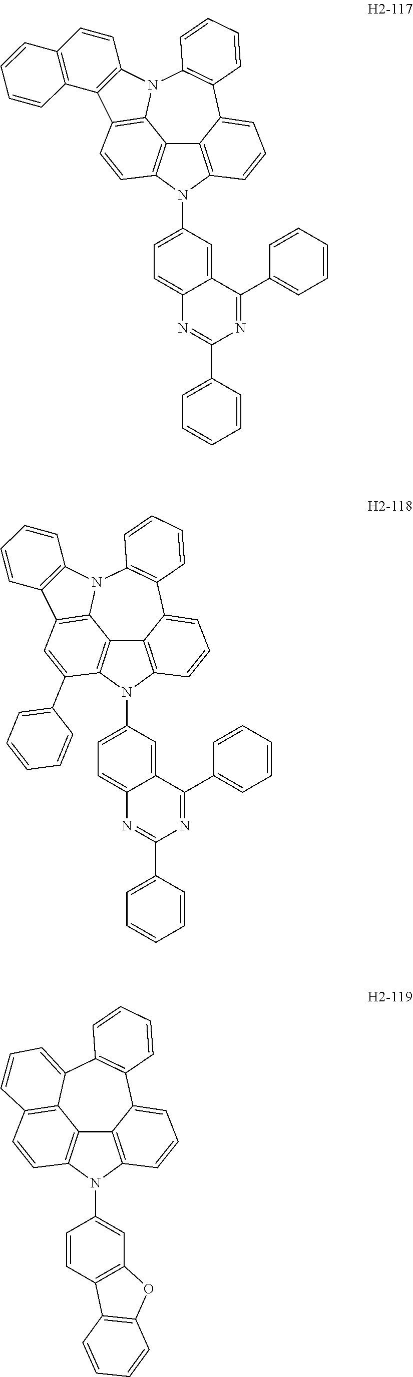 [SMILES: c1ccc(-c2nc(-c3ccccc3)c3cc(-n4c5cccc6c7ccccc7n7c8ccc9ccccc9c8c8ccc4c(c65)c87)ccc3n2)cc1 ; c1ccc(-c2nc(-c3ccccc3)c3cc(-n4c5cccc6c7ccccc7n7c8ccccc8c8cc(-c9ccccc9)c4c(c65)c87)ccc3n2)cc1 ; c1ccc2c(c1)-c1cccc3ccc4c(c13)c1c-2cccc1n4-c1ccc2c(c1)oc1ccccc12]

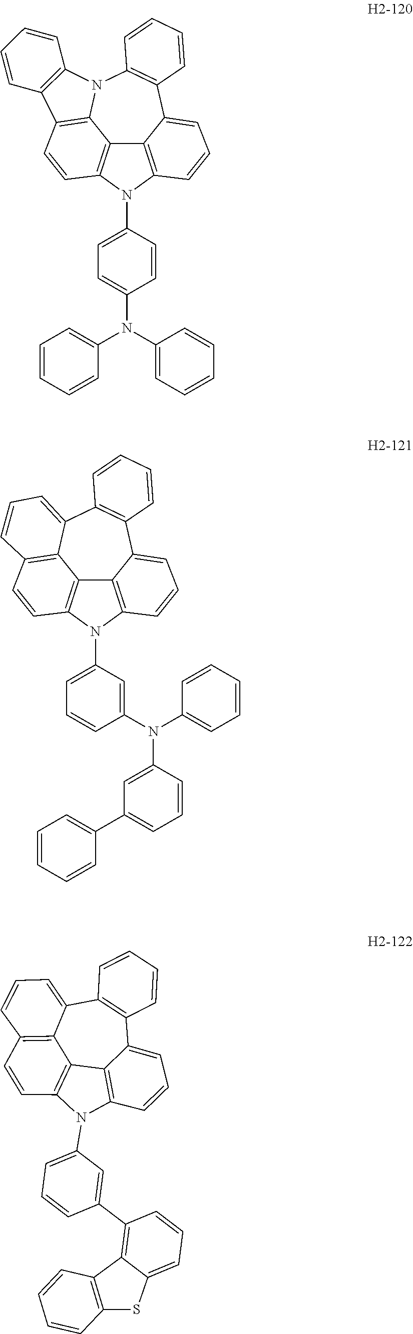 [SMILES: c1ccc(N(c2ccccc2)c2ccc(-n3c4cccc5c6ccccc6n6c7ccccc7c7ccc3c(c54)c76)cc2)cc1 ; c1ccc(-c2cccc(N(c3ccccc3)c3cccc(-n4c5cccc6c5c5c7c(cccc7ccc54)-c4ccccc4-6)c3)c2)cc1 ; c1cc(-c2cccc3sc4ccccc4c23)cc(-n2c3cccc4c3c3c5c(cccc5ccc32)-c2ccccc2-4)c1]

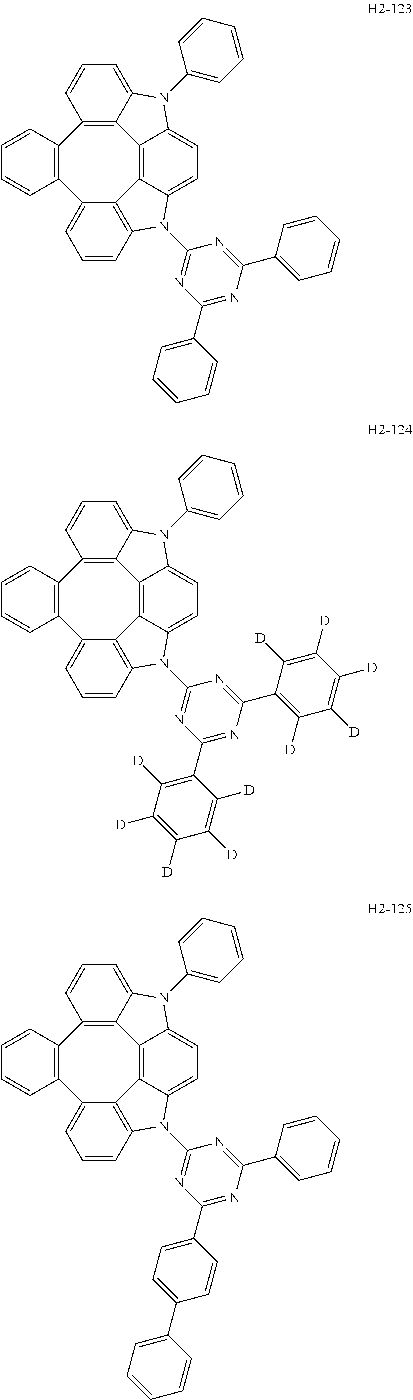 [SMILES: c1ccc(-c2nc(-c3ccccc3)nc(-n3c4cccc5c6ccccc6c6cccc7c6c6c(c54)c3ccc6n7-c3ccccc3)n2)cc1 ; [2H]c1c([2H])c([2H])c(-c2nc(-c3c([2H])c([2H])c([2H])c([2H])c3[2H])nc(-n3c4cccc5c6ccccc6c6cccc7c6c6c(c54)c3ccc6n7-c3ccccc3)n2)c([2H])c1[2H] ; c1ccc(-c2ccc(-c3nc(-c4ccccc4)nc(-n4c5cccc6c7ccccc7c7cccc8c7c7c(c65)c4ccc7n8-c4ccccc4)n3)cc2)cc1]

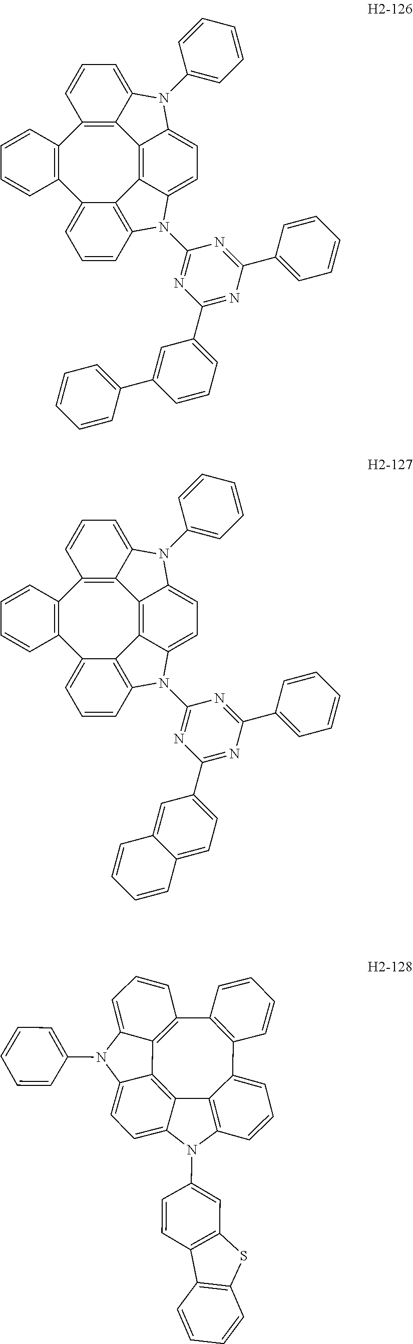 [SMILES: c1ccc(-c2cccc(-c3nc(-c4ccccc4)nc(-n4c5cccc6c7ccccc7c7cccc8c7c7c(c65)c4ccc7n8-c4ccccc4)n3)c2)cc1 ; c1ccc(-c2nc(-c3ccc4ccccc4c3)nc(-n3c4cccc5c6ccccc6c6cccc7c6c6c(c54)c3ccc6n7-c3ccccc3)n2)cc1 ; c1ccc(-n2c3cccc4c5ccccc5c5cccc6c5c5c(c43)c2ccc5n6-c2ccc3c(c2)sc2ccccc23)cc1]

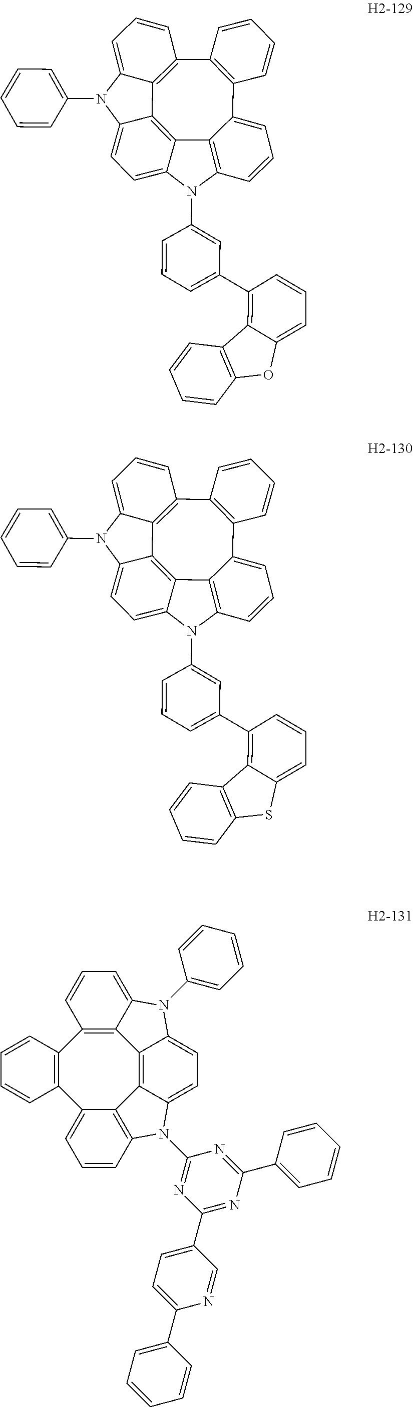 [SMILES: c1ccc(-n2c3cccc4c5ccccc5c5cccc6c5c5c(c43)c2ccc5n6-c2cccc(-c3cccc4oc5ccccc5c34)c2)cc1 ; c1ccc(-n2c3cccc4c5ccccc5c5cccc6c5c5c(c43)c2ccc5n6-c2cccc(-c3cccc4sc5ccccc5c34)c2)cc1 ; c1ccc(-c2ccc(-c3nc(-c4ccccc4)nc(-n4c5cccc6c7ccccc7c7cccc8c7c7c(c65)c4ccc7n8-c4ccccc4)n3)cn2)cc1]

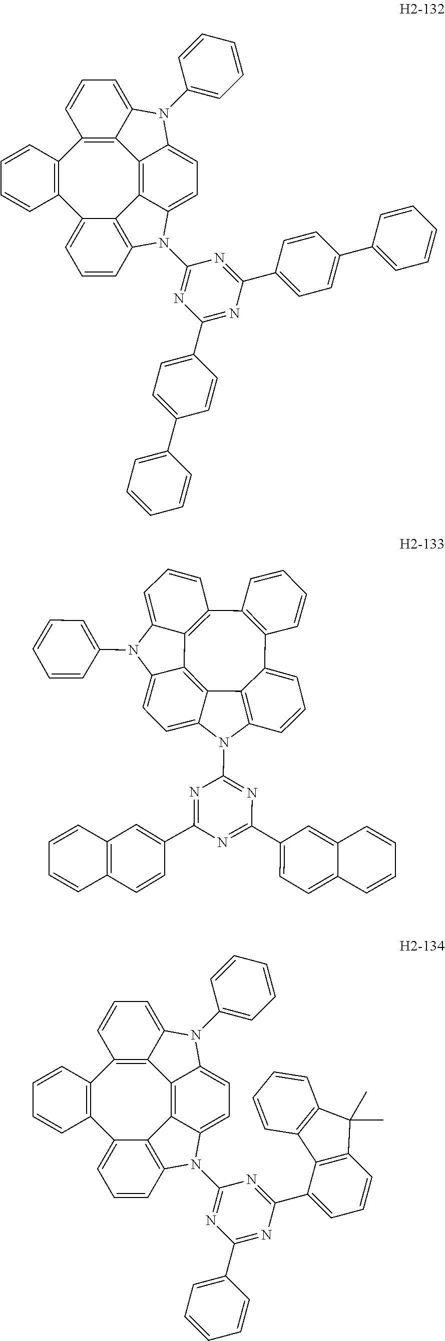 [SMILES: c1ccc(-c2ccc(-c3nc(-c4ccc(-c5ccccc5)cc4)nc(-n4c5cccc6c7ccccc7c7cccc8c7c7c(c65)c4ccc7n8-c4ccccc4)n3)cc2)cc1 ; c1ccc(-n2c3cccc4c5ccccc5c5cccc6c5c5c(c43)c2ccc5n6-c2nc(-c3ccc4ccccc4c3)nc(-c3ccc4ccccc4c3)n2)cc1 ; CC1(C)c2ccccc2-c2c(-c3nc(-c4ccccc4)nc(-n4c5cccc6c7ccccc7c7cccc8c7c7c(c65)c4ccc7n8-c4ccccc4)n3)cccc21]

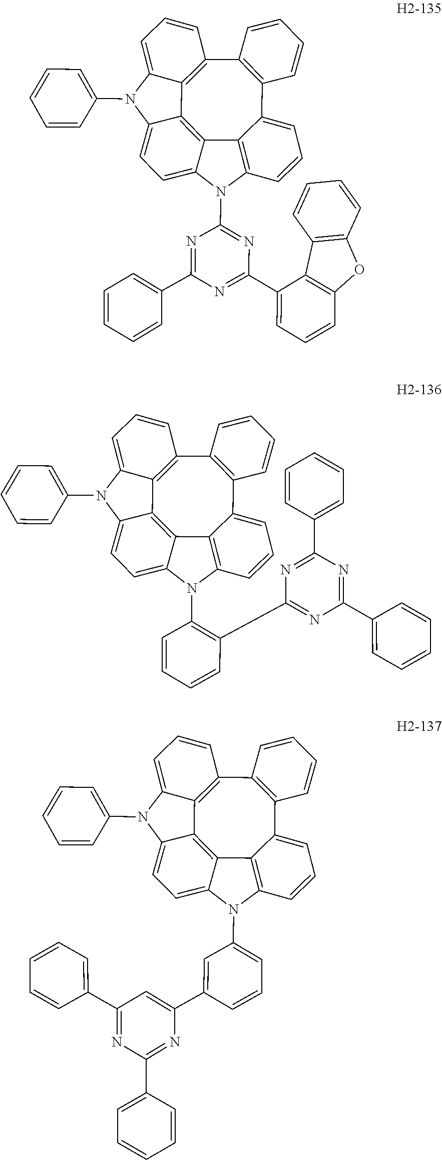

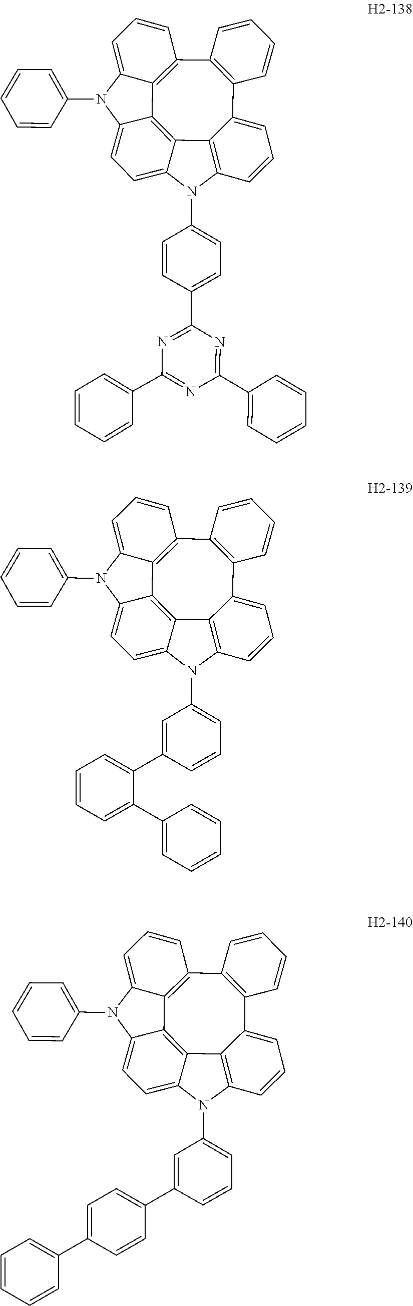 [SMILES: c1ccc(-c2nc(-c3ccccc3)nc(-c3ccc(-n4c5cccc6c7ccccc7c7cccc8c7c7c(c65)c4ccc7n8-c4ccccc4)cc3)n2)cc1 ; c1ccc(-c2ccccc2-c2cccc(-n3c4cccc5c6ccccc6c6cccc7c6c6c(c54)c3ccc6n7-c3ccccc3)c2)cc1 ; c1ccc(-c2ccc(-c3cccc(-n4c5cccc6c7ccccc7c7cccc8c7c7c(c65)c4ccc7n8-c4ccccc4)c3)cc2)cc1]

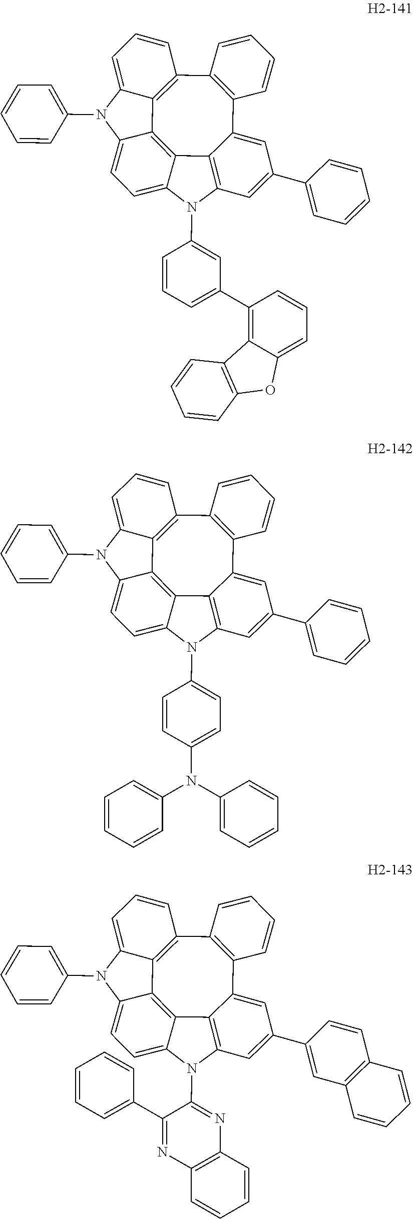 [SMILES: c1ccc(-c2cc3c4ccccc4c4cccc5c4c4c6c3c(c2)n(-c2cccc(-c3cccc7oc8ccccc8c37)c2)c6ccc4n5-c2ccccc2)cc1 ; c1ccc(-c2cc3c4ccccc4c4cccc5c4c4c6c3c(c2)n(-c2ccc(N(c3ccccc3)c3ccccc3)cc2)c6ccc4n5-c2ccccc2)cc1 ; c1ccc(-c2nc3ccccc3nc2-n2c3cc(-c4ccc5ccccc5c4)cc4c5ccccc5c5cccc6c5c5c(c43)c2ccc5n6-c2ccccc2)cc1]

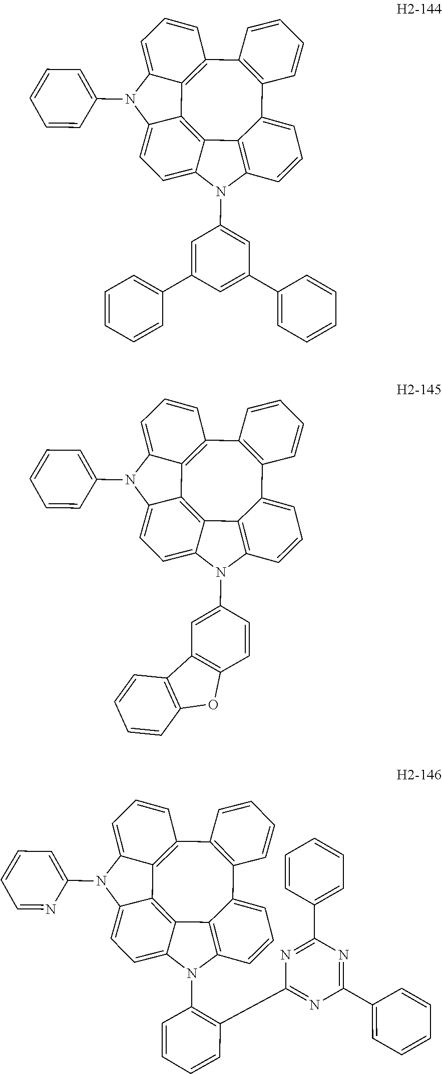 [SMILES: c1ccc(-c2cc(-c3ccccc3)cc(-n3c4cccc5c6ccccc6c6cccc7c6c6c(c54)c3ccc6n7-c3ccccc3)c2)cc1 ; c1ccc(-n2c3cccc4c5ccccc5c5cccc6c5c5c(c43)c2ccc5n6-c2ccc3oc4ccccc4c3c2)cc1 ; c1ccc(-c2nc(-c3ccccc3)nc(-c3ccccc3-n3c4cccc5c6ccccc6c6cccc7c6c6c(c54)c3ccc6n7-c3ccccn3)n2)cc1]

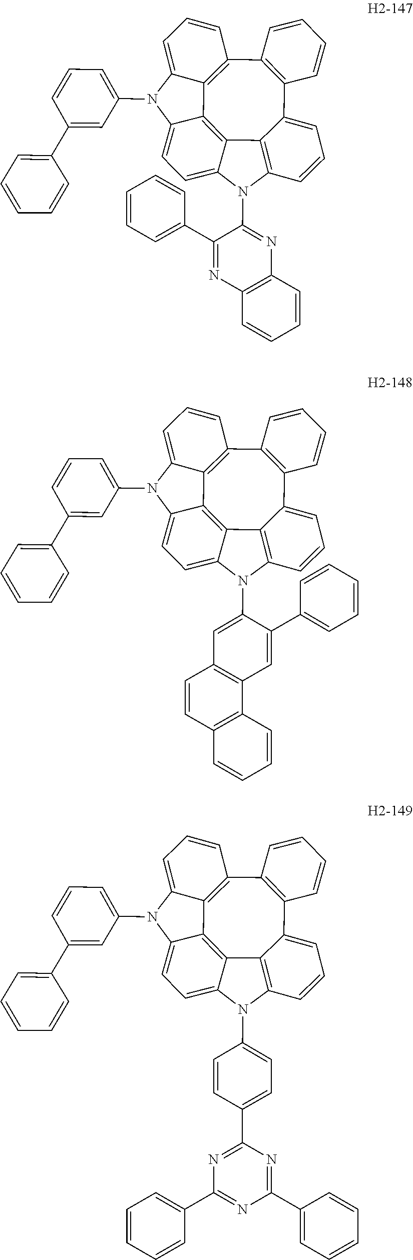 [SMILES: c1ccc(-c2cccc(-n3c4cccc5c6ccccc6c6cccc7c6c6c(c54)c3ccc6n7-c3nc4ccccc4nc3-c3ccccc3)c2)cc1 ; c1ccc(-c2cccc(-n3c4cccc5c6ccccc6c6cccc7c6c6c(c54)c3ccc6n7-c3cc4ccc5ccccc5c4cc3-c3ccccc3)c2)cc1 ; c1ccc(-c2cccc(-n3c4cccc5c6ccccc6c6cccc7c6c6c(c54)c3ccc6n7-c3ccc(-c4nc(-c5ccccc5)nc(-c5ccccc5)n4)cc3)c2)cc1]

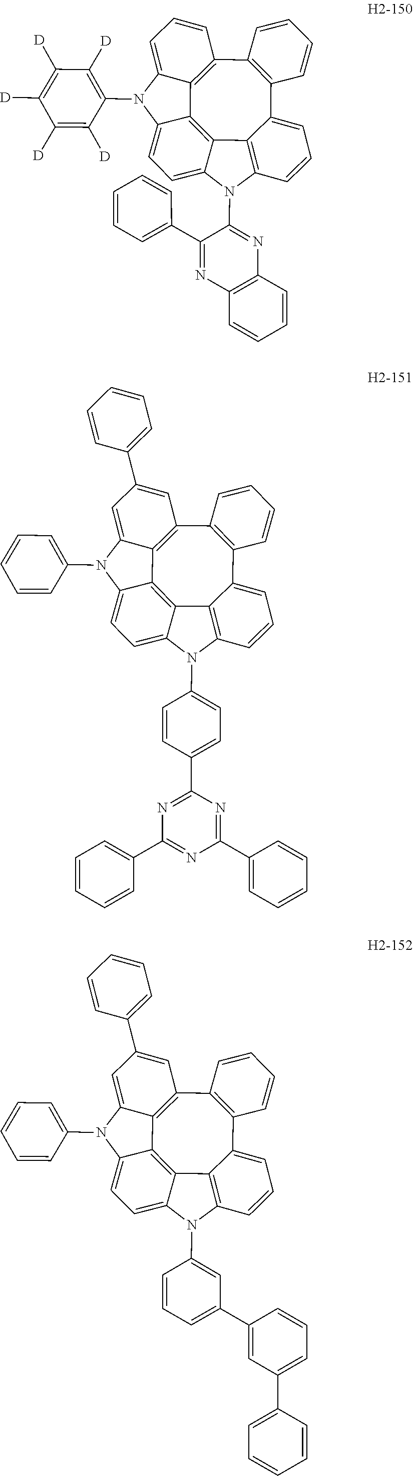 [SMILES: [2H]c1c([2H])c([2H])c(-n2c3cccc4c5ccccc5c5cccc6c5c5c(c43)c2ccc5n6-c2nc3ccccc3nc2-c2ccccc2)c([2H])c1[2H] ; c1ccc(-c2cc3c4ccccc4c4cccc5c4c4c6c3c(c2)n(-c2ccccc2)c6ccc4n5-c2ccc(-c3nc(-c4ccccc4)nc(-c4ccccc4)n3)cc2)cc1 ; c1ccc(-c2cccc(-c3cccc(-n4c5cccc6c7ccccc7c7cc(-c8ccccc8)cc8c7c7c(c65)c4ccc7n8-c4ccccc4)c3)c2)cc1]

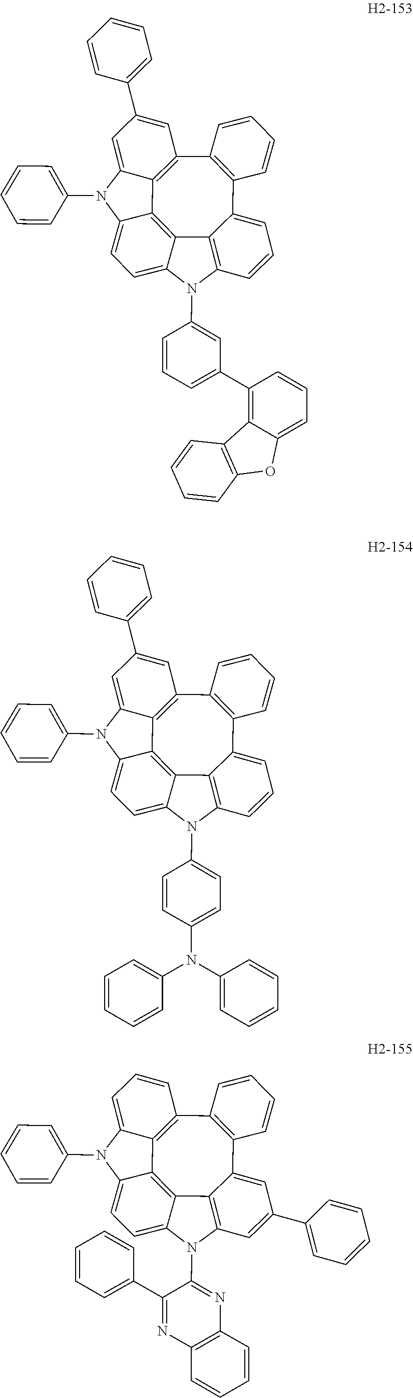 [SMILES: c1ccc(-c2cc3c4ccccc4c4cccc5c4c4c6c3c(c2)n(-c2ccccc2)c6ccc4n5-c2cccc(-c3cccc4oc5ccccc5c34)c2)cc1 ; c1ccc(-c2cc3c4ccccc4c4cccc5c4c4c6c3c(c2)n(-c2ccccc2)c6ccc4n5-c2ccc(N(c3ccccc3)c3ccccc3)cc2)cc1 ; c1ccc(-c2cc3c4ccccc4c4cccc5c4c4c6c3c(c2)n(-c2nc3ccccc3nc2-c2ccccc2)c6ccc4n5-c2ccccc2)cc1]

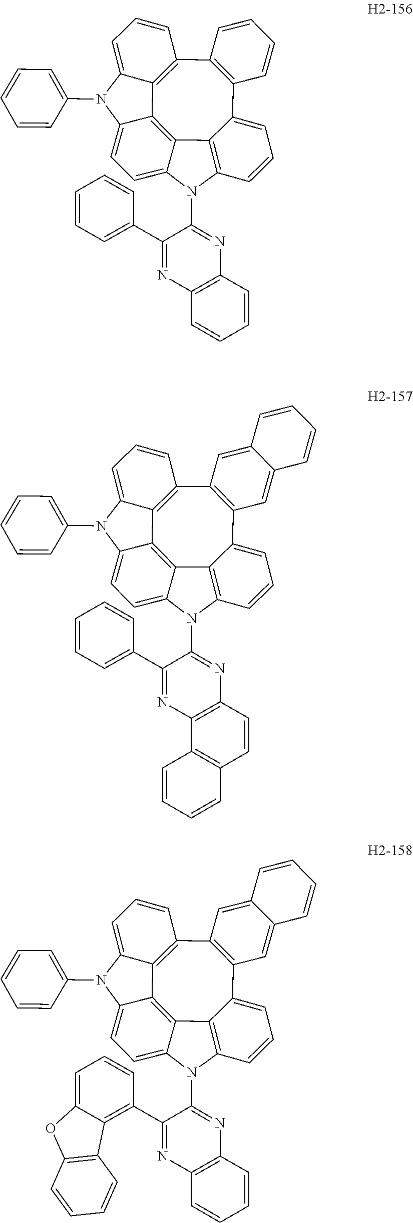 [SMILES: c1ccc(-c2nc3ccccc3nc2-n2c3cccc4c5ccccc5c5cccc6c5c5c(c43)c2ccc5n6-c2ccccc2)cc1 ; c1ccc(-c2nc3c(ccc4ccccc43)nc2-n2c3cccc4c5cc6ccccc6cc5c5cccc6c5c5c(c43)c2ccc5n6-c2ccccc2)cc1 ; c1ccc(-n2c3cccc4c5cc6ccccc6cc5c5cccc6c5c5c(c43)c2ccc5n6-c2nc3ccccc3nc2-c2cccc3oc4ccccc4c23)cc1]

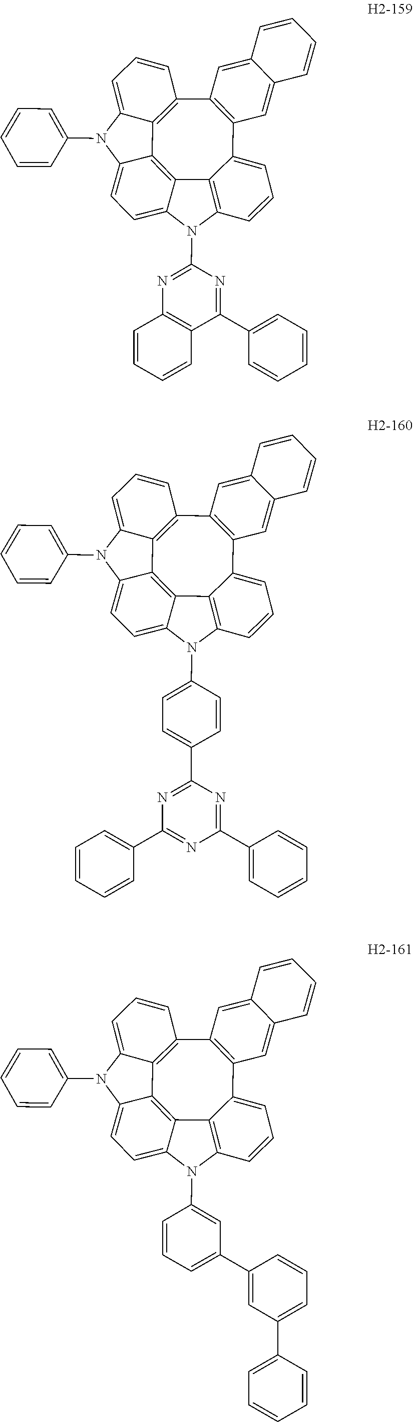 [SMILES: c1ccc(-c2nc(-n3c4cccc5c6cc7ccccc7cc6c6cccc7c6c6c(c54)c3ccc6n7-c3ccccc3)nc3ccccc23)cc1 ; c1ccc(-c2nc(-c3ccccc3)nc(-c3ccc(-n4c5cccc6c7cc8ccccc8cc7c7cccc8c7c7c(c65)c4ccc7n8-c4ccccc4)cc3)n2)cc1 ; c1ccc(-c2cccc(-c3cccc(-n4c5cccc6c7cc8ccccc8cc7c7cccc8c7c7c(c65)c4ccc7n8-c4ccccc4)c3)c2)cc1]

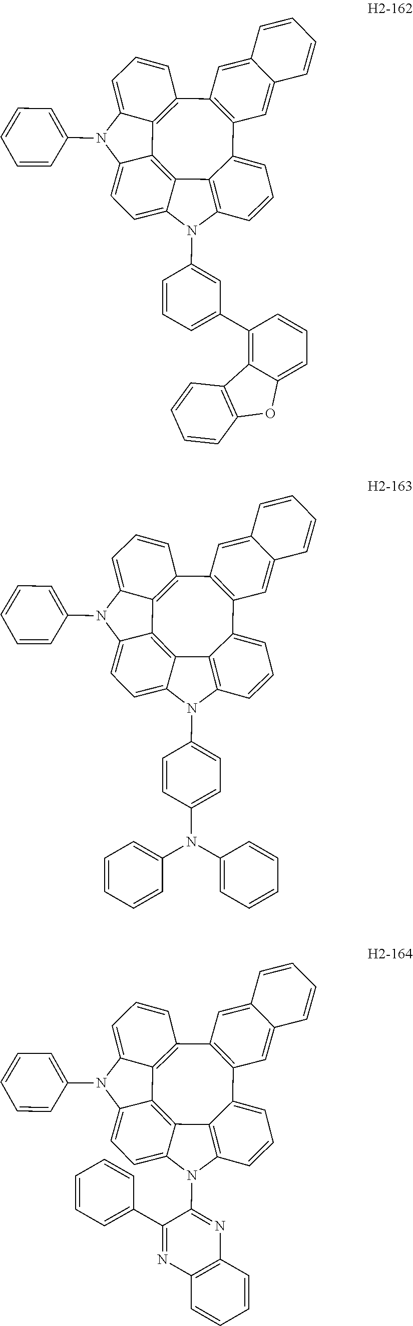 [SMILES: c1ccc(-n2c3cccc4c5cc6ccccc6cc5c5cccc6c5c5c(c43)c2ccc5n6-c2cccc(-c3cccc4oc5ccccc5c34)c2)cc1 ; c1ccc(N(c2ccccc2)c2ccc(-n3c4cccc5c6cc7ccccc7cc6c6cccc7c6c6c(c54)c3ccc6n7-c3ccccc3)cc2)cc1 ; c1ccc(-c2nc3ccccc3nc2-n2c3cccc4c5cc6ccccc6cc5c5cccc6c5c5c(c43)c2ccc5n6-c2ccccc2)cc1]

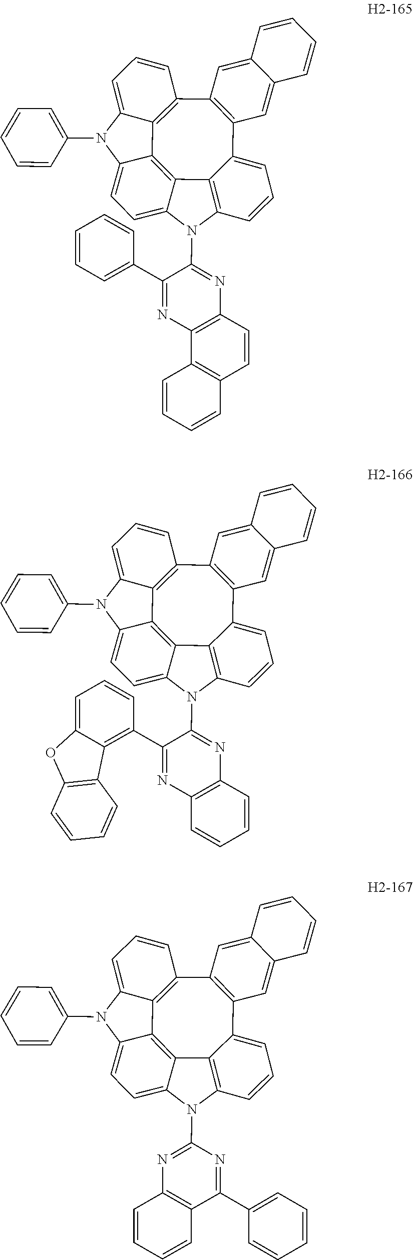 [SMILES: c1ccc(-c2nc3c(ccc4ccccc43)nc2-n2c3cccc4c5cc6ccccc6cc5c5cccc6c5c5c(c43)c2ccc5n6-c2ccccc2)cc1 ; c1ccc(-n2c3cccc4c5cc6ccccc6cc5c5cccc6c5c5c(c43)c2ccc5n6-c2nc3ccccc3nc2-c2cccc3oc4ccccc4c23)cc1 ; c1ccc(-c2nc(-n3c4cccc5c6cc7ccccc7cc6c6cccc7c6c6c(c54)c3ccc6n7-c3ccccc3)nc3ccccc23)cc1]

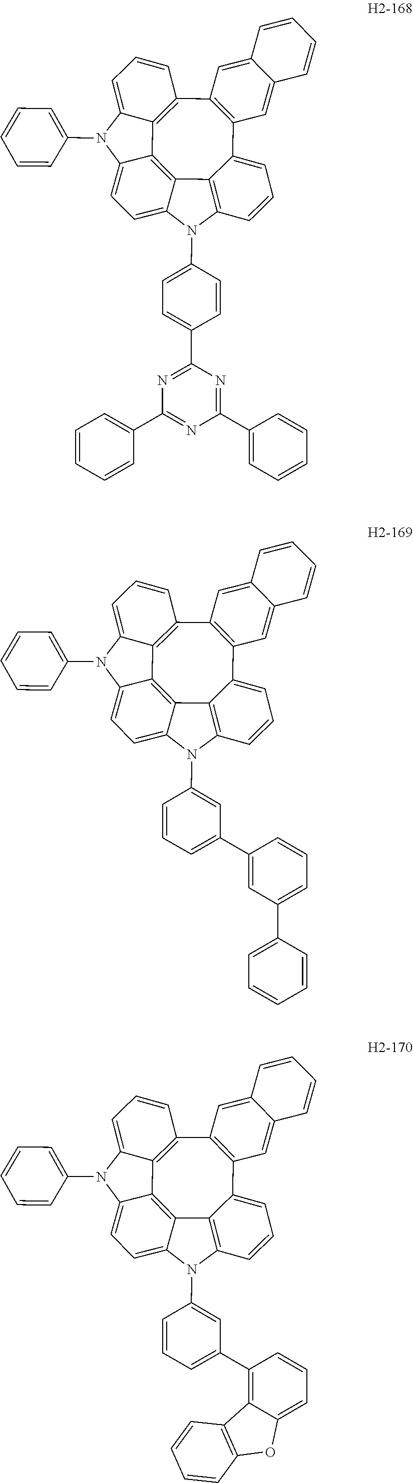 [SMILES: c1ccc(-c2nc(-c3ccccc3)nc(-c3ccc(-n4c5cccc6c7cc8ccccc8cc7c7cccc8c7c7c(c65)c4ccc7n8-c4ccccc4)cc3)n2)cc1 ; c1ccc(-c2cccc(-c3cccc(-n4c5cccc6c7cc8ccccc8cc7c7cccc8c7c7c(c65)c4ccc7n8-c4ccccc4)c3)c2)cc1 ; c1ccc(-n2c3cccc4c5cc6ccccc6cc5c5cccc6c5c5c(c43)c2ccc5n6-c2cccc(-c3cccc4oc5ccccc5c34)c2)cc1]

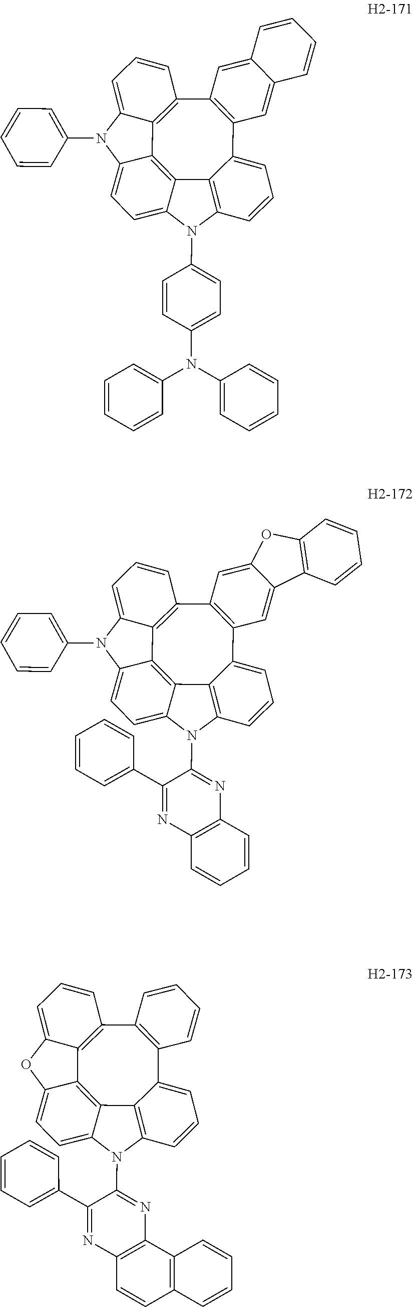 [SMILES: c1ccc(N(c2ccccc2)c2ccc(-n3c4cccc5c6cc7ccccc7cc6c6cccc7c6c6c(c54)c3ccc6n7-c3ccccc3)cc2)cc1 ; c1ccc(-c2nc3ccccc3nc2-n2c3cccc4c5cc6c(cc5c5cccc7c5c5c(c43)c2ccc5n7-c2ccccc2)oc2ccccc26)cc1 ; c1ccc(-c2nc3ccc4ccccc4c3nc2-n2c3cccc4c5ccccc5c5cccc6oc7ccc2c(c7c65)c43)cc1]

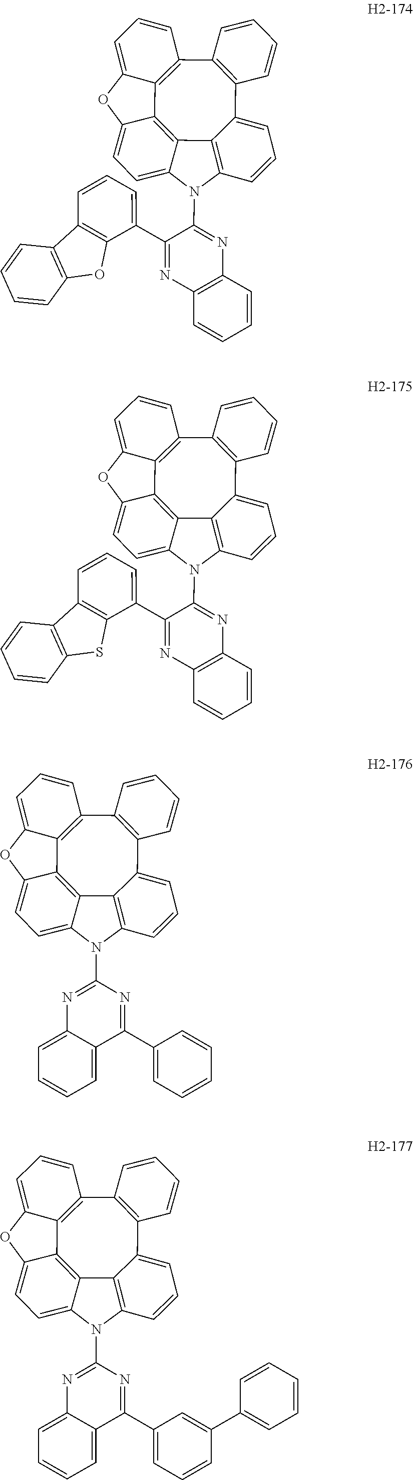 [SMILES: c1ccc2nc(-n3c4cccc5c6ccccc6c6cccc7oc8ccc3c(c8c76)c54)c(-c3cccc4c3oc3ccccc34)nc2c1 ; c1ccc2nc(-n3c4cccc5c6ccccc6c6cccc7oc8ccc3c(c8c76)c54)c(-c3cccc4c3sc3ccccc34)nc2c1 ; c1ccc(-c2nc(-n3c4cccc5c6ccccc6c6cccc7oc8ccc3c(c8c76)c54)nc3ccccc23)cc1 ; c1ccc(-c2cccc(-c3nc(-n4c5cccc6c7ccccc7c7cccc8oc9ccc4c(c9c87)c65)nc4ccccc34)c2)cc1]

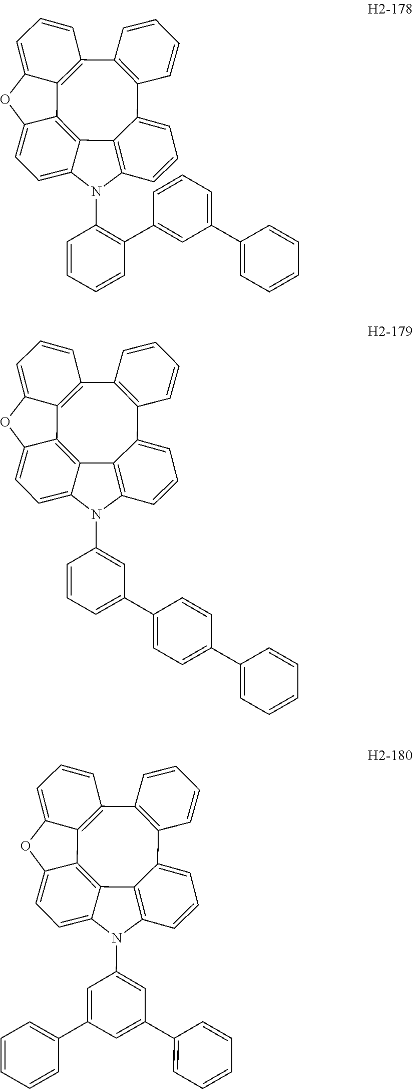 [SMILES: c1ccc(-c2cccc(-c3ccccc3-n3c4cccc5c6ccccc6c6cccc7oc8ccc3c(c8c76)c54)c2)cc1 ; c1ccc(-c2ccc(-c3cccc(-n4c5cccc6c7ccccc7c7cccc8oc9ccc4c(c9c87)c65)c3)cc2)cc1 ; c1ccc(-c2cc(-c3ccccc3)cc(-n3c4cccc5c6ccccc6c6cccc7oc8ccc3c(c8c76)c54)c2)cc1]

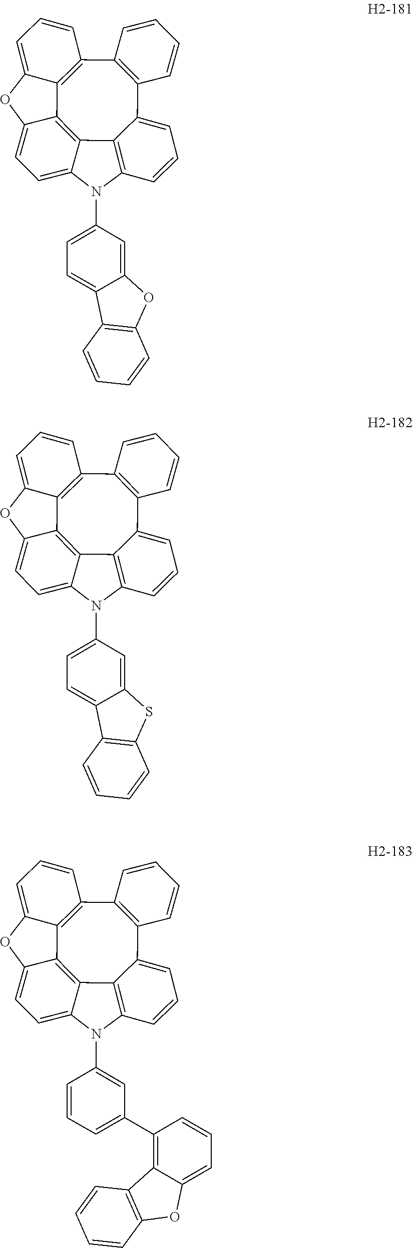 [SMILES: c1ccc2c(c1)oc1cc(-n3c4cccc5c6ccccc6c6cccc7oc8ccc3c(c8c76)c54)ccc12 ; c1ccc2c(c1)sc1cc(-n3c4cccc5c6ccccc6c6cccc7oc8ccc3c(c8c76)c54)ccc12 ; c1cc(-c2cccc3oc4ccccc4c23)cc(-n2c3cccc4c5ccccc5c5cccc6oc7ccc2c(c7c65)c43)c1]

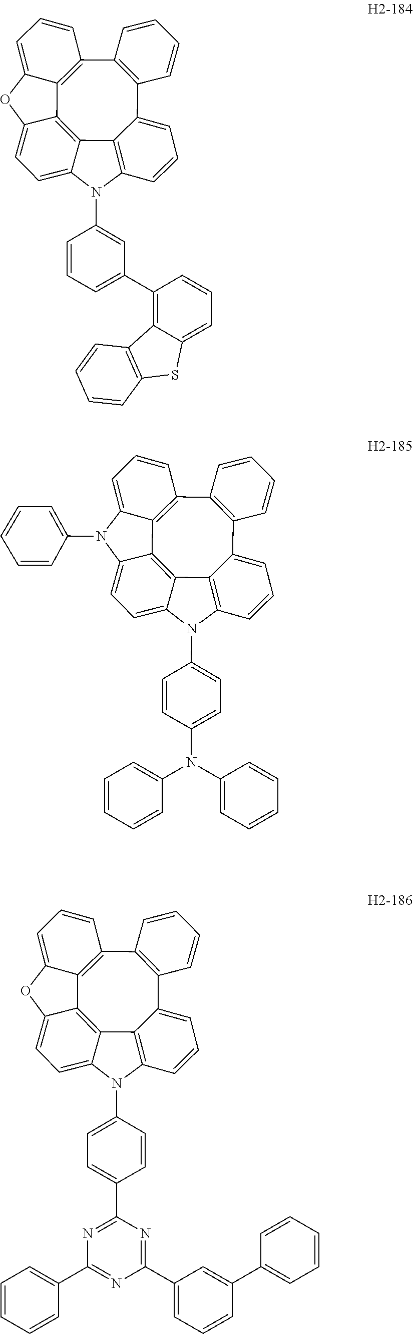 [SMILES: c1cc(-c2cccc3sc4ccccc4c23)cc(-n2c3cccc4c5ccccc5c5cccc6oc7ccc2c(c7c65)c43)c1 ; c1ccc(N(c2ccccc2)c2ccc(-n3c4cccc5c6ccccc6c6cccc7c6c6c(c54)c3ccc6n7-c3ccccc3)cc2)cc1 ; c1ccc(-c2cccc(-c3nc(-c4ccccc4)nc(-c4ccc(-n5c6cccc7c8ccccc8c8cccc9oc%10ccc5c(c%10c98)c76)cc4)n3)c2)cc1]

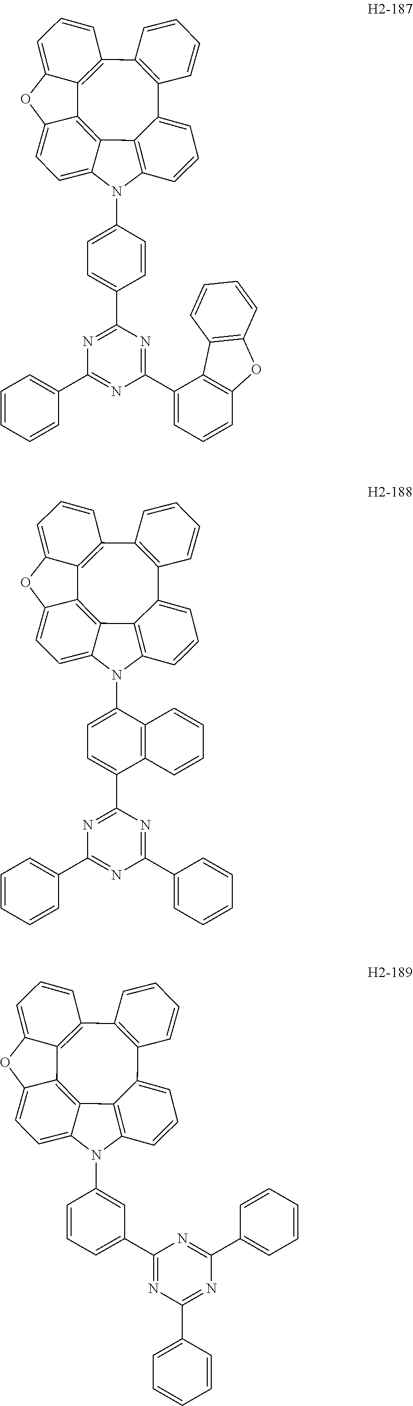 [SMILES: c1ccc(-c2nc(-c3ccc(-n4c5cccc6c7ccccc7c7cccc8oc9ccc4c(c9c87)c65)cc3)nc(-c3cccc4oc5ccccc5c34)n2)cc1 ; c1ccc(-c2nc(-c3ccccc3)nc(-c3ccc(-n4c5cccc6c7ccccc7c7cccc8oc9ccc4c(c9c87)c65)c4ccccc34)n2)cc1 ; c1ccc(-c2nc(-c3ccccc3)nc(-c3cccc(-n4c5cccc6c7ccccc7c7cccc8oc9ccc4c(c9c87)c65)c3)n2)cc1]

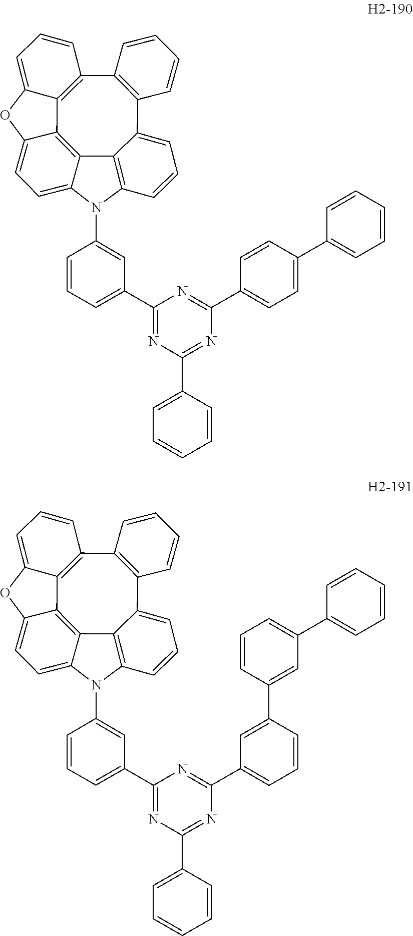 [SMILES: c1ccc(-c2ccc(-c3nc(-c4ccccc4)nc(-c4cccc(-n5c6cccc7c8ccccc8c8cccc9oc%10ccc5c(c%10c98)c76)c4)n3)cc2)cc1 ; c1ccc(-c2cccc(-c3cccc(-c4nc(-c5ccccc5)nc(-c5cccc(-n6c7cccc8c9ccccc9c9cccc%10oc%11ccc6c(c%11c%109)c87)c5)n4)c3)c2)cc1]

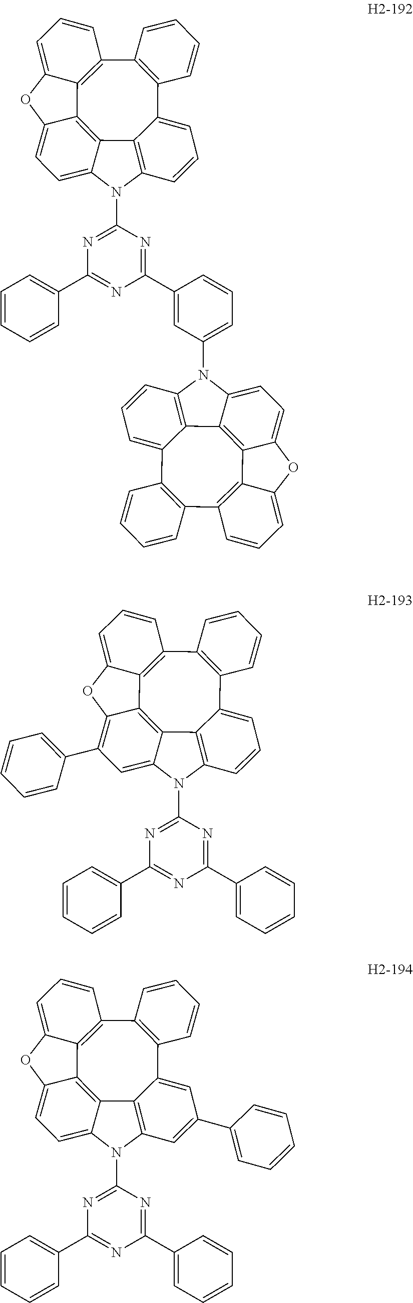 [SMILES: c1ccc(-c2nc(-c3cccc(-n4c5cccc6c7ccccc7c7cccc8oc9ccc4c(c9c87)c65)c3)nc(-n3c4cccc5c6ccccc6c6cccc7oc8ccc3c(c8c76)c54)n2)cc1 ; c1ccc(-c2nc(-c3ccccc3)nc(-n3c4cccc5c6ccccc6c6cccc7oc8c(-c9ccccc9)cc3c(c8c76)c54)n2)cc1 ; c1ccc(-c2cc3c4ccccc4c4cccc5oc6ccc7c(c6c54)c3c(c2)n7-c2nc(-c3ccccc3)nc(-c3ccccc3)n2)cc1]

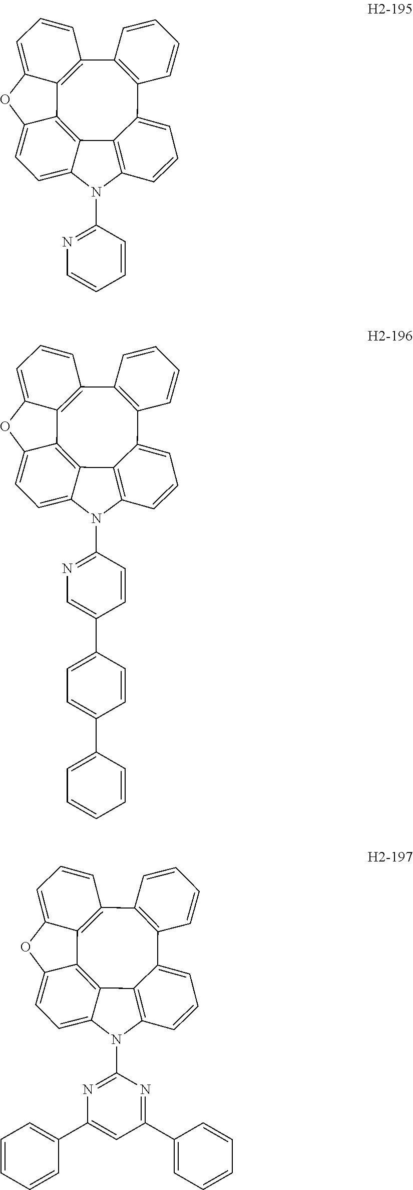 [SMILES: c1ccc(-n2c3cccc4c5ccccc5c5cccc6oc7ccc2c(c7c65)c43)nc1 ; c1ccc(-c2ccc(-c3ccc(-n4c5cccc6c7ccccc7c7cccc8oc9ccc4c(c9c87)c65)nc3)cc2)cc1 ; c1ccc(-c2cc(-c3ccccc3)nc(-n3c4cccc5c6ccccc6c6cccc7oc8ccc3c(c8c76)c54)n2)cc1]

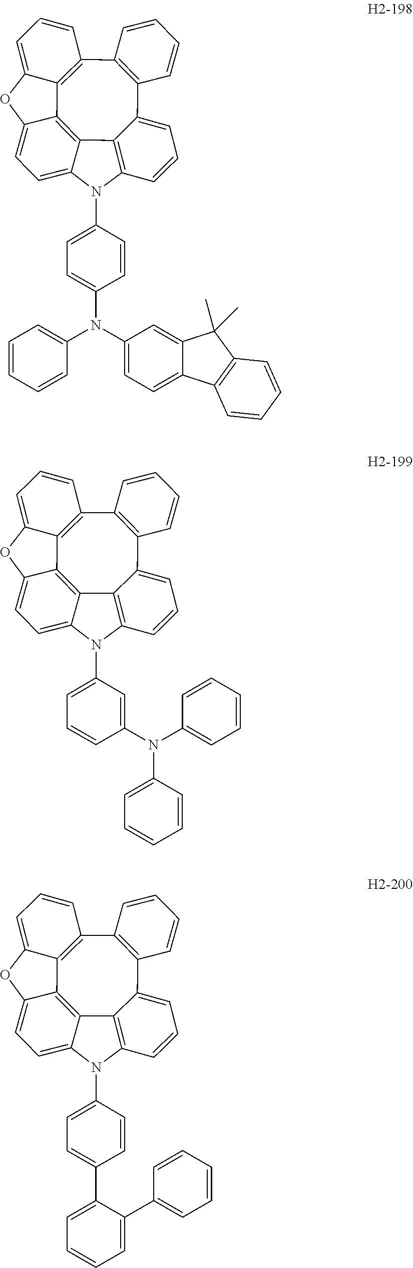 [SMILES: CC1(C)c2ccccc2-c2ccc(N(c3ccccc3)c3ccc(-n4c5cccc6c7ccccc7c7cccc8oc9ccc4c(c9c87)c65)cc3)cc21 ; c1ccc(N(c2ccccc2)c2cccc(-n3c4cccc5c6ccccc6c6cccc7oc8ccc3c(c8c76)c54)c2)cc1 ; c1ccc(-c2ccccc2-c2ccc(-n3c4cccc5c6ccccc6c6cccc7oc8ccc3c(c8c76)c54)cc2)cc1]

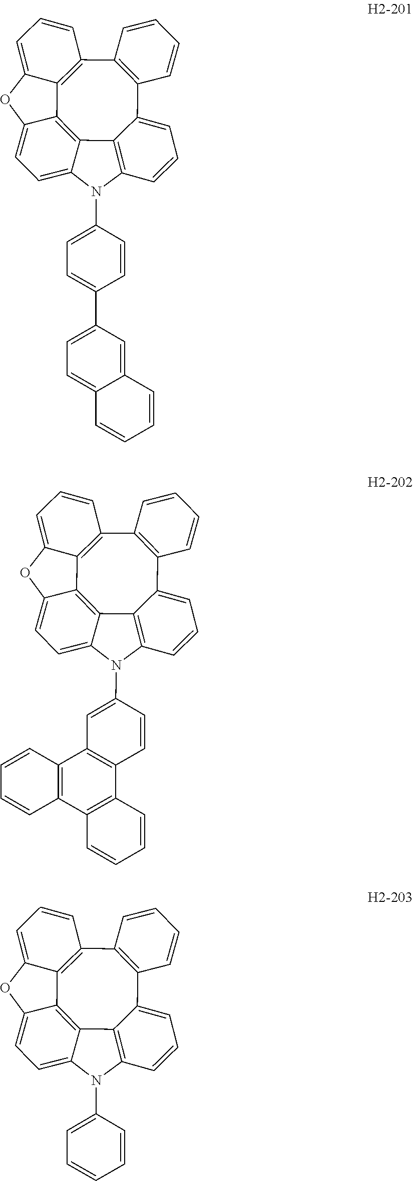 [SMILES: c1ccc2cc(-c3ccc(-n4c5cccc6c7ccccc7c7cccc8oc9ccc4c(c9c87)c65)cc3)ccc2c1 ; c1ccc2c(c1)c1ccccc1c1cc(-n3c4cccc5c6ccccc6c6cccc7oc8ccc3c(c8c76)c54)ccc21 ; c1ccc(-n2c3cccc4c5ccccc5c5cccc6oc7ccc2c(c7c65)c43)cc1]

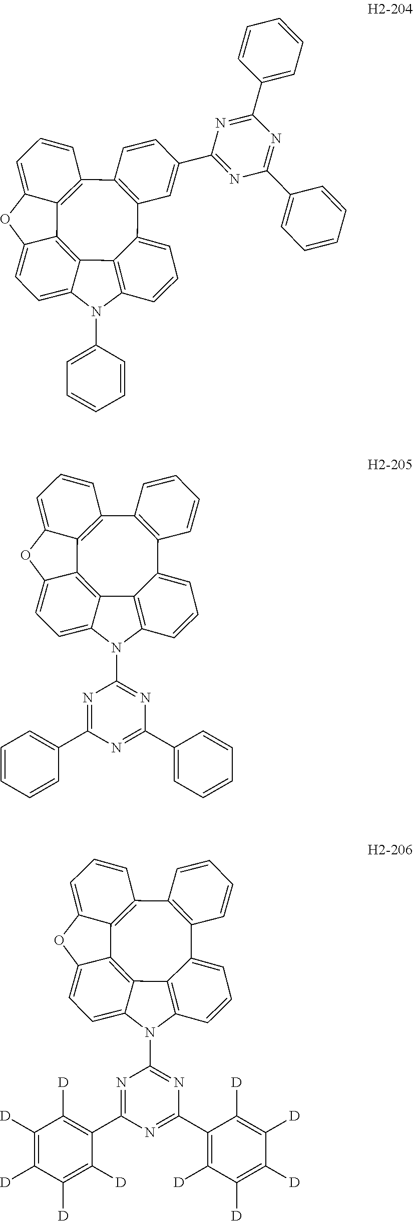 [SMILES: c1ccc(-c2nc(-c3ccccc3)nc(-c3ccc4c(c3)c3cccc5c3c3c6c(ccc3n5-c3ccccc3)oc3cccc4c36)n2)cc1 ; c1ccc(-c2nc(-c3ccccc3)nc(-n3c4cccc5c6ccccc6c6cccc7oc8ccc3c(c8c76)c54)n2)cc1 ; [2H]c1c([2H])c([2H])c(-c2nc(-c3c([2H])c([2H])c([2H])c([2H])c3[2H])nc(-n3c4cccc5c6ccccc6c6cccc7oc8ccc3c(c8c76)c54)n2)c([2H])c1[2H]]

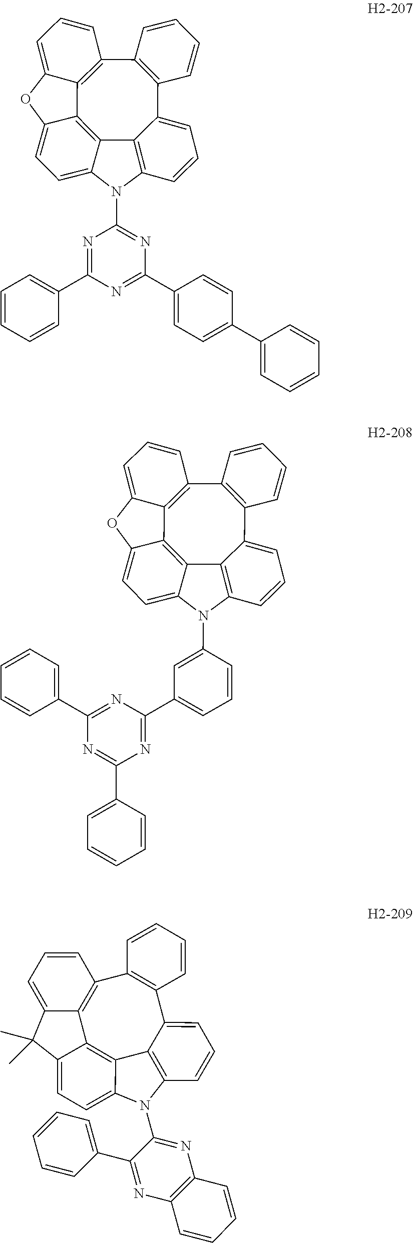 [SMILES: c1ccc(-c2ccc(-c3nc(-c4ccccc4)nc(-n4c5cccc6c7ccccc7c7cccc8oc9ccc4c(c9c87)c65)n3)cc2)cc1 ; c1ccc(-c2nc(-c3ccccc3)nc(-c3cccc(-n4c5cccc6c7ccccc7c7cccc8oc9ccc4c(c9c87)c65)c3)n2)cc1 ; CC1(C)c2cccc3c4ccccc4c4cccc5c4c4c(c1ccc4n5-c1nc4ccccc4nc1-c1ccccc1)c23]

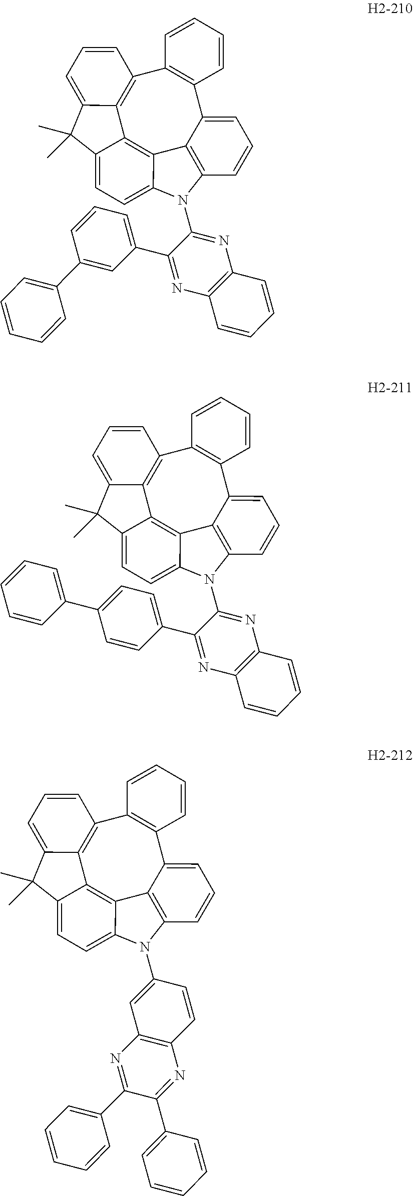 [SMILES: CC1(C)c2cccc3c4ccccc4c4cccc5c4c4c(c1ccc4n5-c1nc4ccccc4nc1-c1cccc(-c4ccccc4)c1)c23 ; CC1(C)c2cccc3c4ccccc4c4cccc5c4c4c(c1ccc4n5-c1nc4ccccc4nc1-c1ccc(-c4ccccc4)cc1)c23 ; CC1(C)c2cccc3c4ccccc4c4cccc5c4c4c(c1ccc4n5-c1ccc4nc(-c5ccccc5)c(-c5ccccc5)nc4c1)c23]

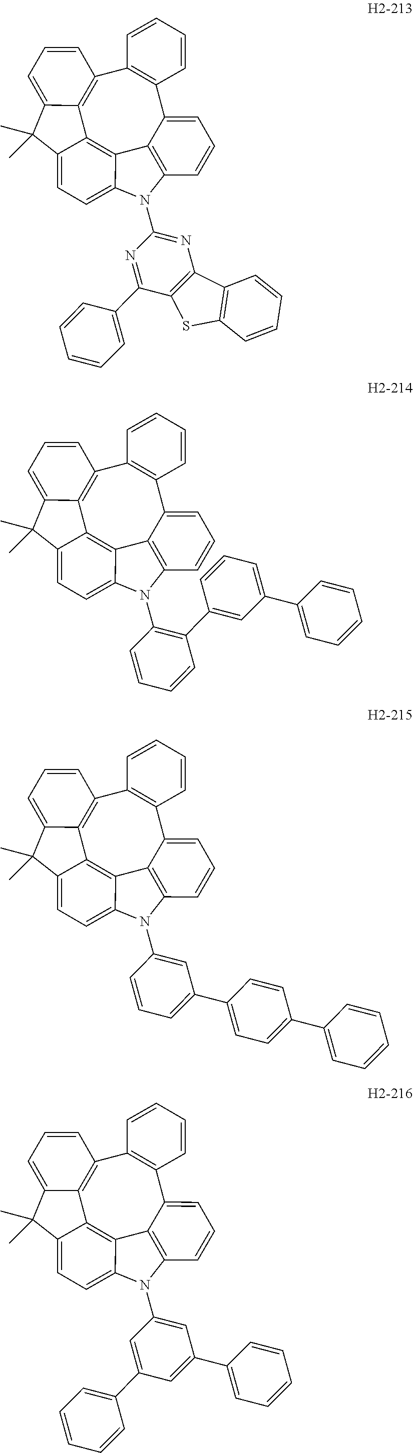 [SMILES: CC1(C)c2cccc3c4ccccc4c4cccc5c4c4c(c1ccc4n5-c1nc(-c4ccccc4)c4sc5ccccc5c4n1)c23 ; CC1(C)c2cccc3c4ccccc4c4cccc5c4c4c(c1ccc4n5-c1ccccc1-c1cccc(-c4ccccc4)c1)c23 ; CC1(C)c2cccc3c4ccccc4c4cccc5c4c4c(c1ccc4n5-c1cccc(-c4ccc(-c5ccccc5)cc4)c1)c23 ; CC1(C)c2cccc3c4ccccc4c4cccc5c4c4c(c1ccc4n5-c1cc(-c4ccccc4)cc(-c4ccccc4)c1)c23]

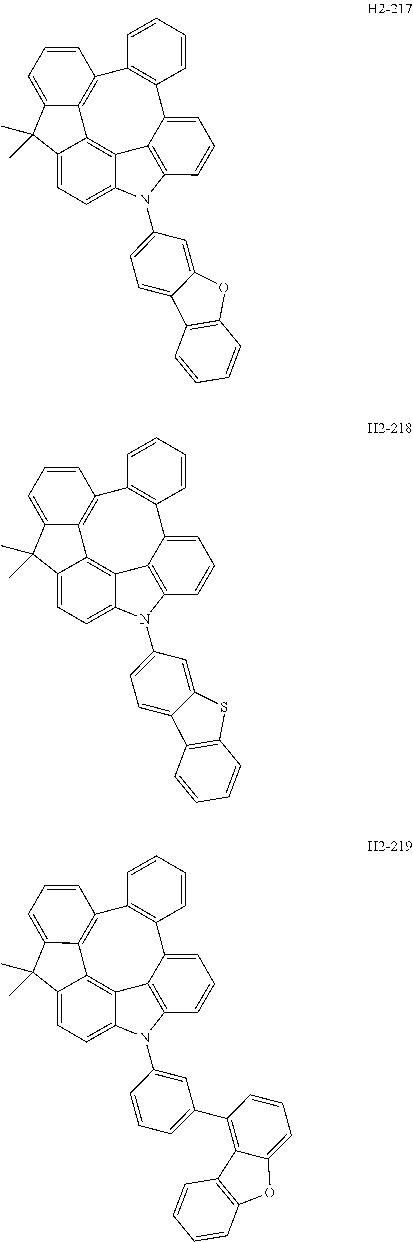 [SMILES: CC1(C)c2cccc3c4ccccc4c4cccc5c4c4c(c1ccc4n5-c1ccc4c(c1)oc1ccccc14)c23 ; CC1(C)c2cccc3c4ccccc4c4cccc5c4c4c(c1ccc4n5-c1ccc4c(c1)sc1ccccc14)c23 ; CC1(C)c2cccc3c4ccccc4c4cccc5c4c4c(c1ccc4n5-c1cccc(-c4cccc5oc6ccccc6c45)c1)c23]

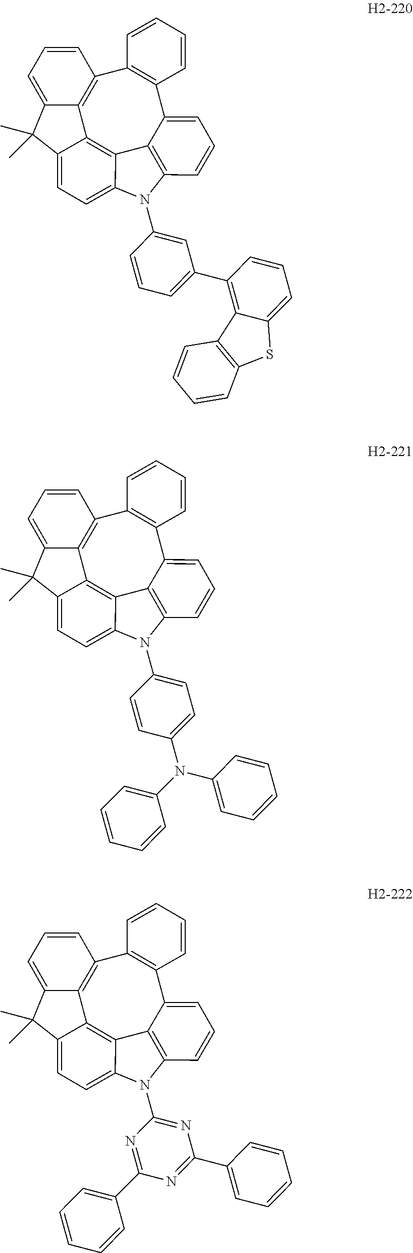 [SMILES: CC1(C)c2cccc3c4ccccc4c4cccc5c4c4c(c1ccc4n5-c1cccc(-c4cccc5sc6ccccc6c45)c1)c23 ; CC1(C)c2cccc3c4ccccc4c4cccc5c4c4c(c1ccc4n5-c1ccc(N(c4ccccc4)c4ccccc4)cc1)c23 ; CC1(C)c2cccc3c4ccccc4c4cccc5c4c4c(c1ccc4n5-c1nc(-c4ccccc4)nc(-c4ccccc4)n1)c23]

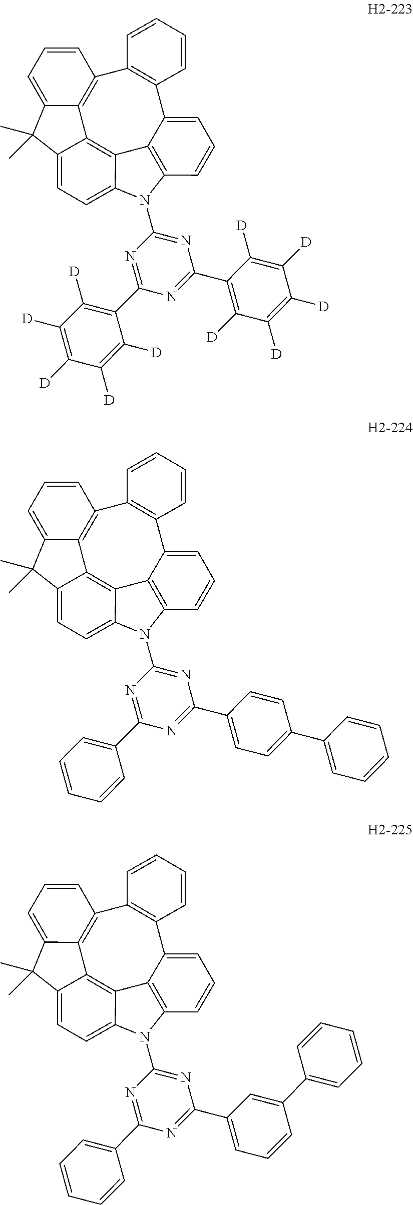 [SMILES: [2H]c1c([2H])c([2H])c(-c2nc(-c3c([2H])c([2H])c([2H])c([2H])c3[2H])nc(-n3c4cccc5c6ccccc6c6cccc7c6c6c(ccc3c6c54)C7(C)C)n2)c([2H])c1[2H] ; CC1(C)c2cccc3c4ccccc4c4cccc5c4c4c(c1ccc4n5-c1nc(-c4ccccc4)nc(-c4ccc(-c5ccccc5)cc4)n1)c23 ; CC1(C)c2cccc3c4ccccc4c4cccc5c4c4c(c1ccc4n5-c1nc(-c4ccccc4)nc(-c4cccc(-c5ccccc5)c4)n1)c23]

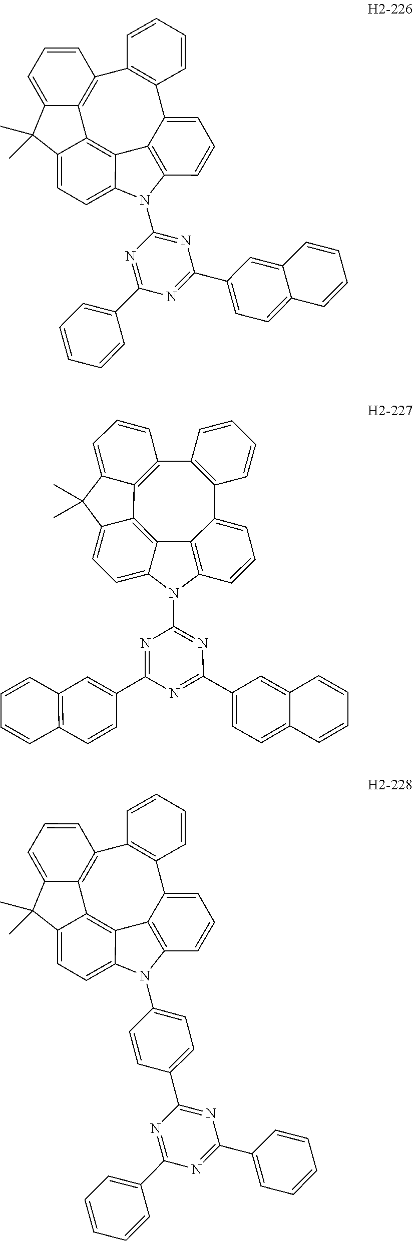 [SMILES: CC1(C)c2cccc3c4ccccc4c4cccc5c4c4c(c1ccc4n5-c1nc(-c4ccccc4)nc(-c4ccc5ccccc5c4)n1)c23 ; CC1(C)c2cccc3c4ccccc4c4cccc5c4c4c(c1ccc4n5-c1nc(-c4ccc5ccccc5c4)nc(-c4ccc5ccccc5c4)n1)c23 ; CC1(C)c2cccc3c4ccccc4c4cccc5c4c4c(c1ccc4n5-c1ccc(-c4nc(-c5ccccc5)nc(-c5ccccc5)n4)cc1)c23]

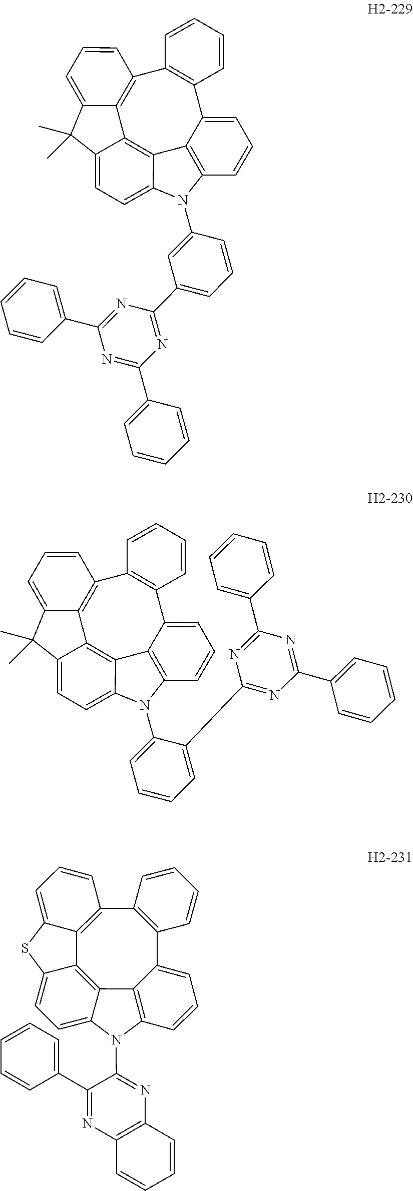 [SMILES: CC1(C)c2cccc3c4ccccc4c4cccc5c4c4c(c1ccc4n5-c1cccc(-c4nc(-c5ccccc5)nc(-c5ccccc5)n4)c1)c23 ; CC1(C)c2cccc3c4ccccc4c4cccc5c4c4c(c1ccc4n5-c1ccccc1-c1nc(-c4ccccc4)nc(-c4ccccc4)n1)c23 ; c1ccc(-c2nc3ccccc3nc2-n2c3cccc4c5ccccc5c5cccc6sc7ccc2c(c7c65)c43)cc1]

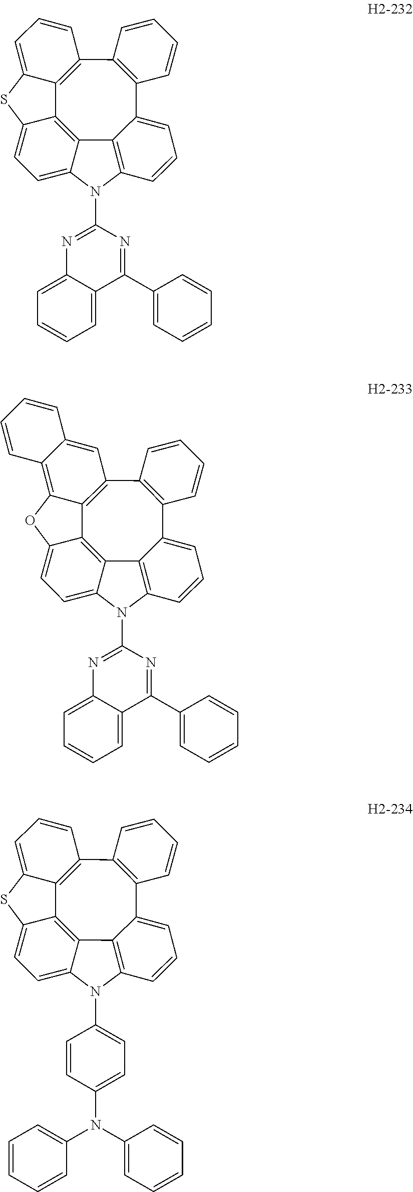 [SMILES: c1ccc(-c2nc(-n3c4cccc5c6ccccc6c6cccc7sc8ccc3c(c8c76)c54)nc3ccccc23)cc1 ; c1ccc(-c2nc(-n3c4cccc5c6ccccc6c6cc7ccccc7c7oc8ccc3c(c8c67)c54)nc3ccccc23)cc1 ; c1ccc(N(c2ccccc2)c2ccc(-n3c4cccc5c6ccccc6c6cccc7sc8ccc3c(c8c76)c54)cc2)cc1]

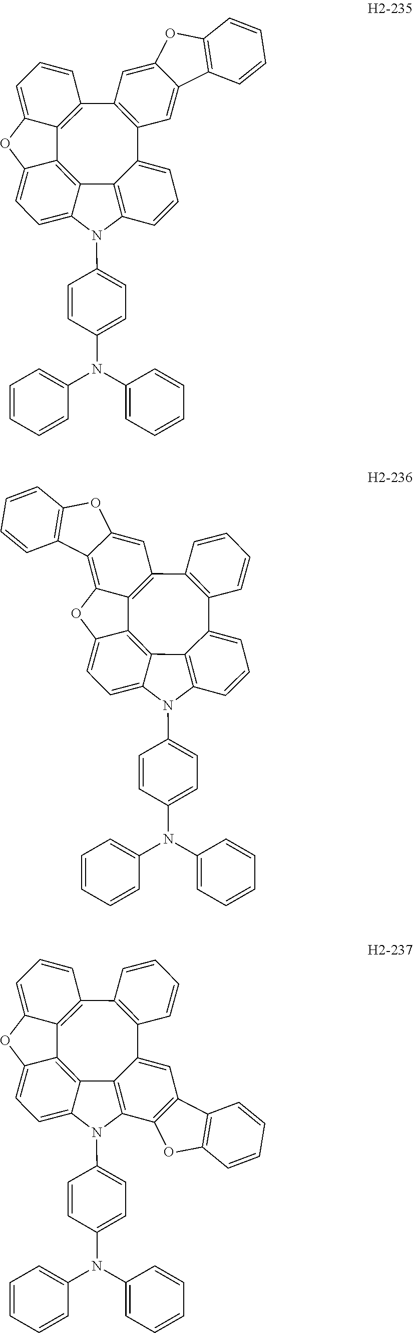 [SMILES: c1ccc(N(c2ccccc2)c2ccc(-n3c4cccc5c6cc7c(cc6c6cccc8oc9ccc3c(c9c86)c54)oc3ccccc37)cc2)cc1 ; c1ccc(N(c2ccccc2)c2ccc(-n3c4cccc5c6ccccc6c6cc7oc8ccccc8c7c7oc8ccc3c(c8c67)c54)cc2)cc1 ; c1ccc(N(c2ccccc2)c2ccc(-n3c4ccc5oc6cccc7c8ccccc8c8cc9c%10ccccc%10oc9c3c8c4c5c67)cc2)cc1]

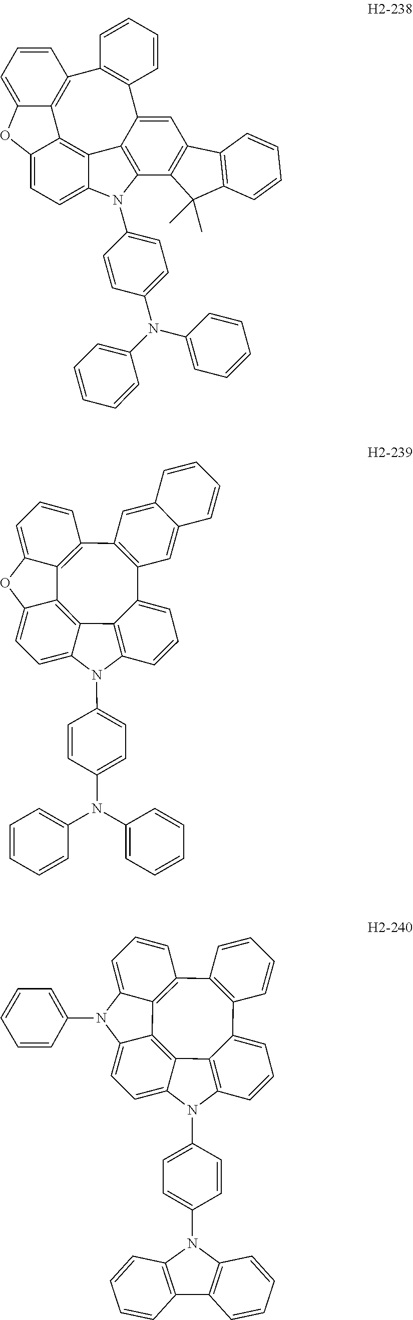 [SMILES: CC1(C)c2ccccc2-c2cc3c4ccccc4c4cccc5oc6ccc7c(c6c54)c3c(c21)n7-c1ccc(N(c2ccccc2)c2ccccc2)cc1 ; c1ccc(N(c2ccccc2)c2ccc(-n3c4cccc5c6cc7ccccc7cc6c6cccc7oc8ccc3c(c8c76)c54)cc2)cc1 ; c1ccc(-n2c3cccc4c5ccccc5c5cccc6c5c5c(c43)c2ccc5n6-c2ccc(-n3c4ccccc4c4ccccc43)cc2)cc1]

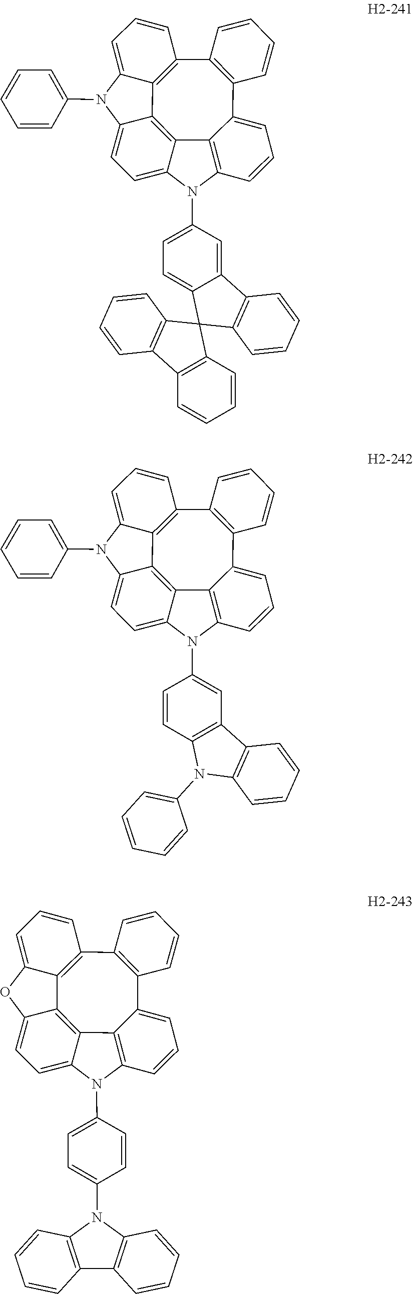 [SMILES: c1ccc(-n2c3cccc4c5ccccc5c5cccc6c5c5c(c43)c2ccc5n6-c2ccc3c(c2)-c2ccccc2C32c3ccccc3-c3ccccc32)cc1 ; c1ccc(-n2c3ccccc3c3cc(-n4c5cccc6c7ccccc7c7cccc8c7c7c(c65)c4ccc7n8-c4ccccc4)ccc32)cc1 ; c1ccc2c(c1)c1cccc3oc4ccc5c(c4c31)c1c2cccc1n5-c1ccc(-n2c3ccccc3c3ccccc32)cc1]

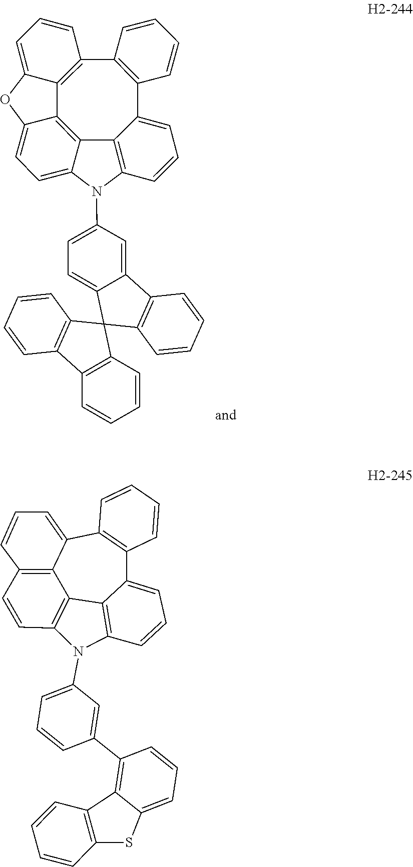 [SMILES: c1ccc2c(c1)-c1ccccc1C21c2ccccc2-c2cc(-n3c4cccc5c6ccccc6c6cccc7oc8ccc3c(c8c76)c54)ccc21 ; c1cc(-c2cccc3sc4ccccc4c23)cc(-n2c3cccc4c3c3c5c(cccc5ccc32)-c2ccccc2-4)c1]

16. A plurality of host materials comprising a first host material and a second host material, wherein the first host material comprises an organic electroluminescent compound according to claim 1 and the second host material comprises an organic electroluminescent compound represented by the following formula 4: 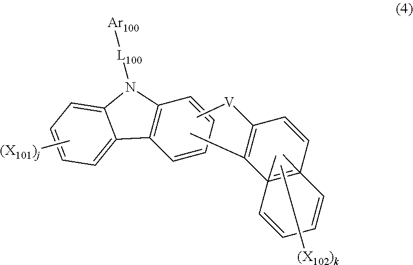
wherein,
V represents CX₁₁X₁₂, NX₁₃, O, or S;
L₁₀₀ represents a single bond, a substituted or unsubstituted (C6-C30)arylene, or a substituted or unsubstituted (3- to 30-membered)heteroarylene;
Ar₁₀₀ represents a substituted or unsubstituted (C6-C30)aryl, a substituted or unsubstituted (3- to 30-membered)heteroaryl, or —NX₉X₁₀;
X₉ and X₁₀ each independently represent a substituted or unsubstituted (C1-C30)alkyl, a substituted or unsubstituted (C3-C30)cycloalkyl, a substituted or unsubstituted (C6-C30)aryl, or a substituted or unsubstituted (3- to 30-membered)heteroaryl;
X₁₁ to X₁₃, X₁₀₁, and X₁₀₂ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted (C2-C30)alkenyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(3- to 30-membered)heteroarylamino, or a substituted or unsubstituted (C6-C30)aryl(3- to 30-membered)heteroarylamino;
j is an integer of 1 to 4, and k is an integer of 1 to 6; and
when j and k are 2 or more, each of X₁₀₁ and each of X₁₀₂ may be the same or different.

17. The plurality of host materials according to claim 16, wherein the compound represented by formula 4 is selected from the following compounds:

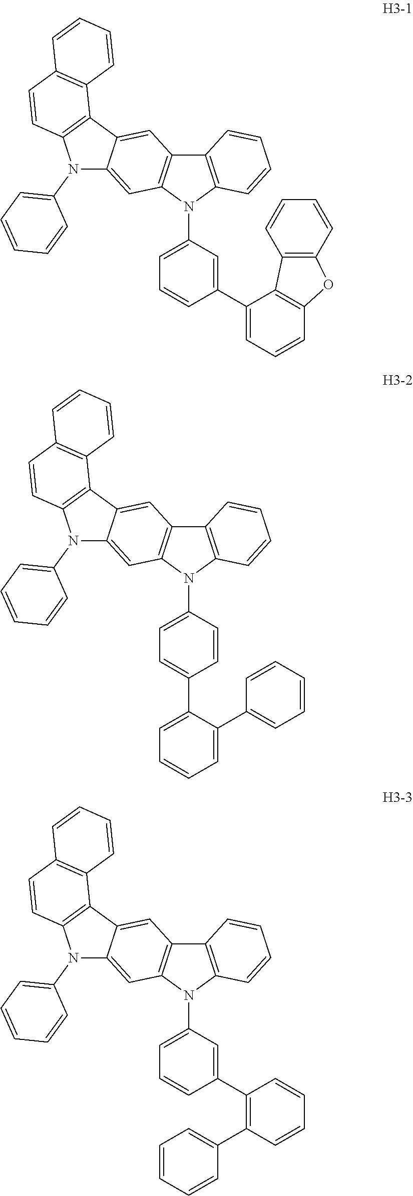 [SMILES: c1ccc(-n2c3cc4c(cc3c3c5ccccc5ccc32)c2ccccc2n4-c2cccc(-c3cccc4oc5ccccc5c34)c2)cc1 ; c1ccc(-c2ccccc2-c2ccc(-n3c4ccccc4c4cc5c6c7ccccc7ccc6n(-c6ccccc6)c5cc43)cc2)cc1 ; c1ccc(-c2ccccc2-c2cccc(-n3c4ccccc4c4cc5c6c7ccccc7ccc6n(-c6ccccc6)c5cc43)c2)cc1]

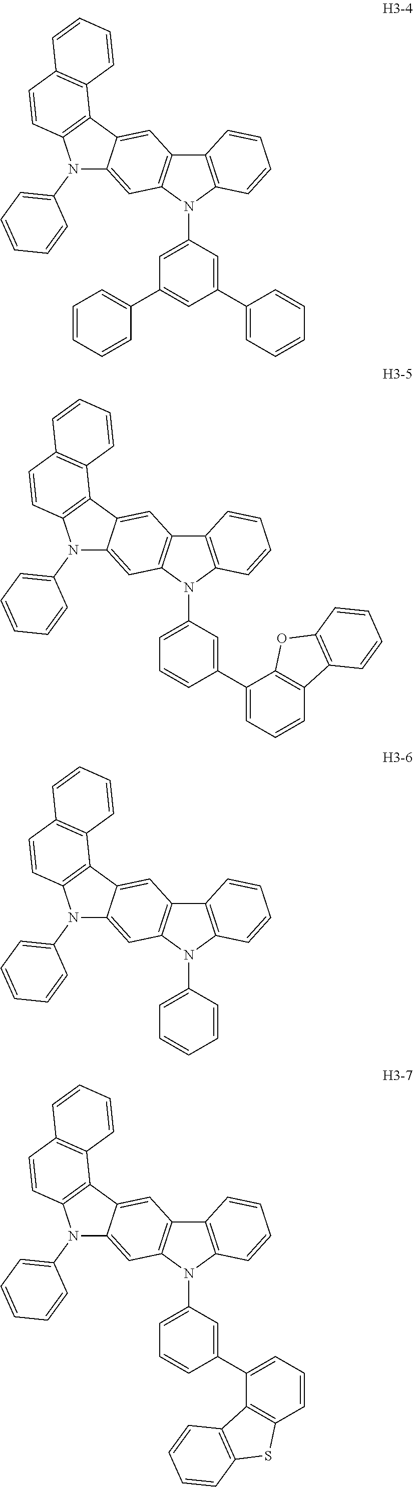 [SMILES: c1ccc(-c2cc(-c3ccccc3)cc(-n3c4ccccc4c4cc5c6c7ccccc7ccc6n(-c6ccccc6)c5cc43)c2)cc1 ; c1ccc(-n2c3cc4c(cc3c3c5ccccc5ccc32)c2ccccc2n4-c2cccc(-c3cccc4c3oc3ccccc34)c2)cc1 ; c1ccc(-n2c3ccccc3c3cc4c5c6ccccc6ccc5n(-c5ccccc5)c4cc32)cc1 ; c1ccc(-n2c3cc4c(cc3c3c5ccccc5ccc32)c2ccccc2n4-c2cccc(-c3cccc4sc5ccccc5c34)c2)cc1]

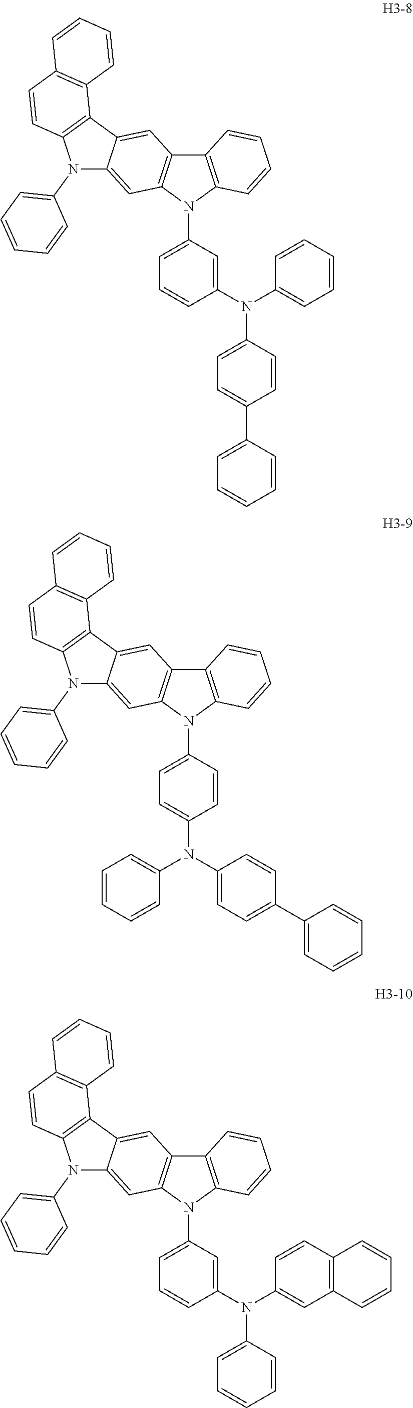 [SMILES: c1ccc(-c2ccc(N(c3ccccc3)c3cccc(-n4c5ccccc5c5cc6c7c8ccccc8ccc7n(-c7ccccc7)c6cc54)c3)cc2)cc1 ; c1ccc(-c2ccc(N(c3ccccc3)c3ccc(-n4c5ccccc5c5cc6c7c8ccccc8ccc7n(-c7ccccc7)c6cc54)cc3)cc2)cc1 ; c1ccc(N(c2cccc(-n3c4ccccc4c4cc5c6c7ccccc7ccc6n(-c6ccccc6)c5cc43)c2)c2ccc3ccccc3c2)cc1]

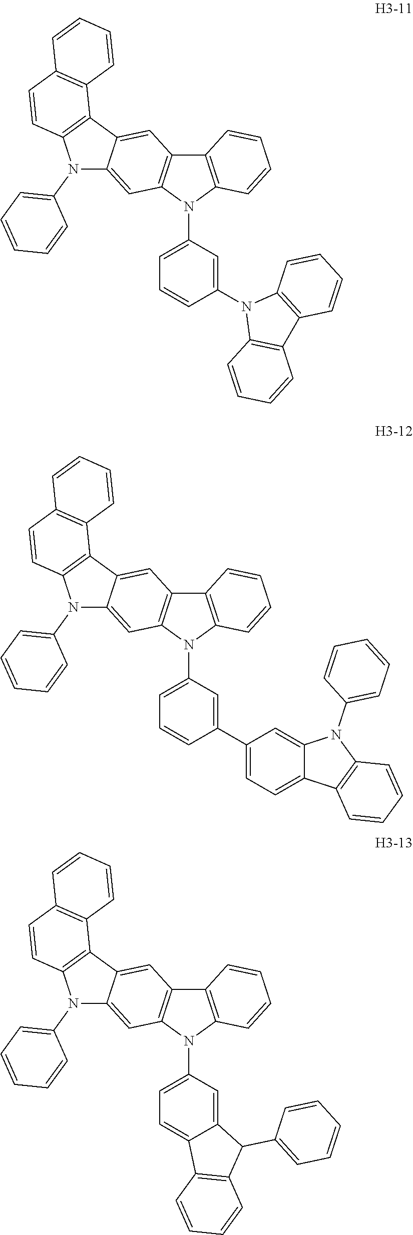 [SMILES: c1ccc(-n2c3cc4c(cc3c3c5ccccc5ccc32)c2ccccc2n4-c2cccc(-n3c4ccccc4c4ccccc43)c2)cc1 ; c1ccc(-n2c3ccccc3c3ccc(-c4cccc(-n5c6ccccc6c6cc7c8c9ccccc9ccc8n(-c8ccccc8)c7cc65)c4)cc32)cc1 ; c1ccc(C2c3ccccc3-c3ccc(-n4c5ccccc5c5cc6c7c8ccccc8ccc7n(-c7ccccc7)c6cc54)cc32)cc1]

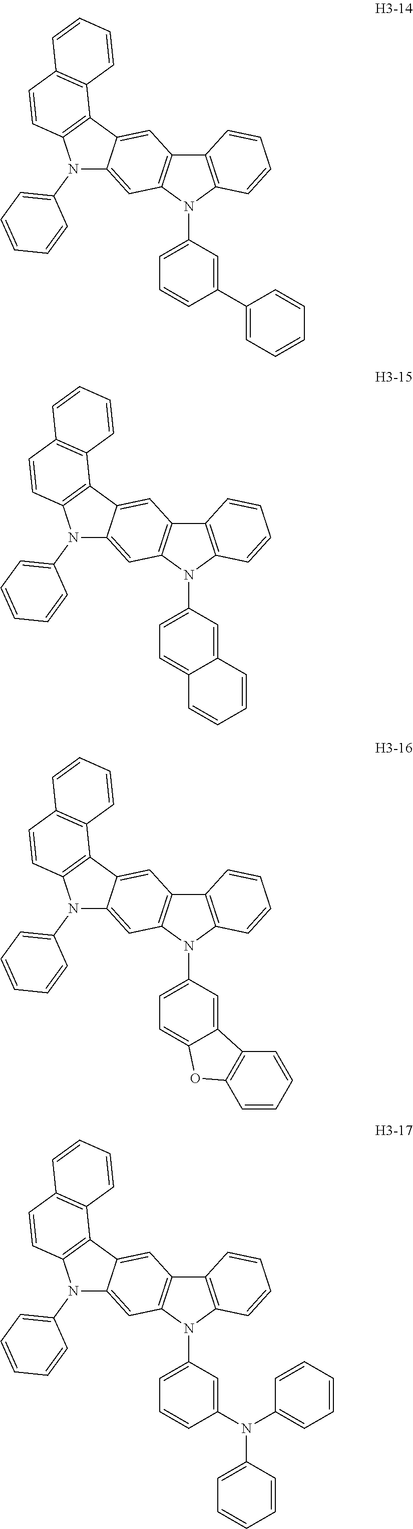 [SMILES: c1ccc(-c2cccc(-n3c4ccccc4c4cc5c6c7ccccc7ccc6n(-c6ccccc6)c5cc43)c2)cc1 ; c1ccc(-n2c3cc4c(cc3c3c5ccccc5ccc32)c2ccccc2n4-c2ccc3ccccc3c2)cc1 ; c1ccc(-n2c3cc4c(cc3c3c5ccccc5ccc32)c2ccccc2n4-c2ccc3oc4ccccc4c3c2)cc1 ; c1ccc(N(c2ccccc2)c2cccc(-n3c4ccccc4c4cc5c6c7ccccc7ccc6n(-c6ccccc6)c5cc43)c2)cc1]

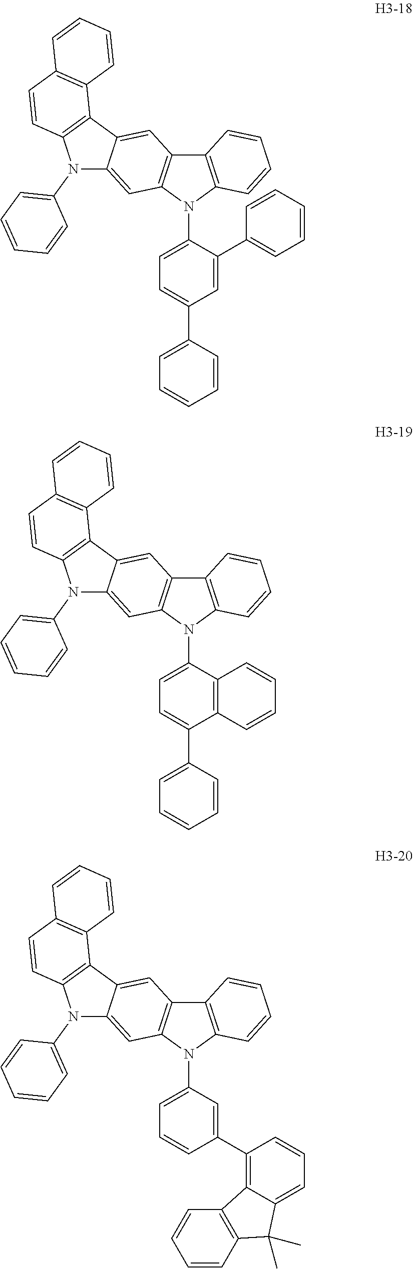 [SMILES: c1ccc(-c2ccc(-n3c4ccccc4c4cc5c6c7ccccc7ccc6n(-c6ccccc6)c5cc43)c(-c3ccccc3)c2)cc1 ; c1ccc(-c2ccc(-n3c4ccccc4c4cc5c6c7ccccc7ccc6n(-c6ccccc6)c5cc43)c3ccccc23)cc1 ; CC1(C)c2ccccc2-c2c(-c3cccc(-n4c5ccccc5c5cc6c7c8ccccc8ccc7n(-c7ccccc7)c6cc54)c3)cccc21]

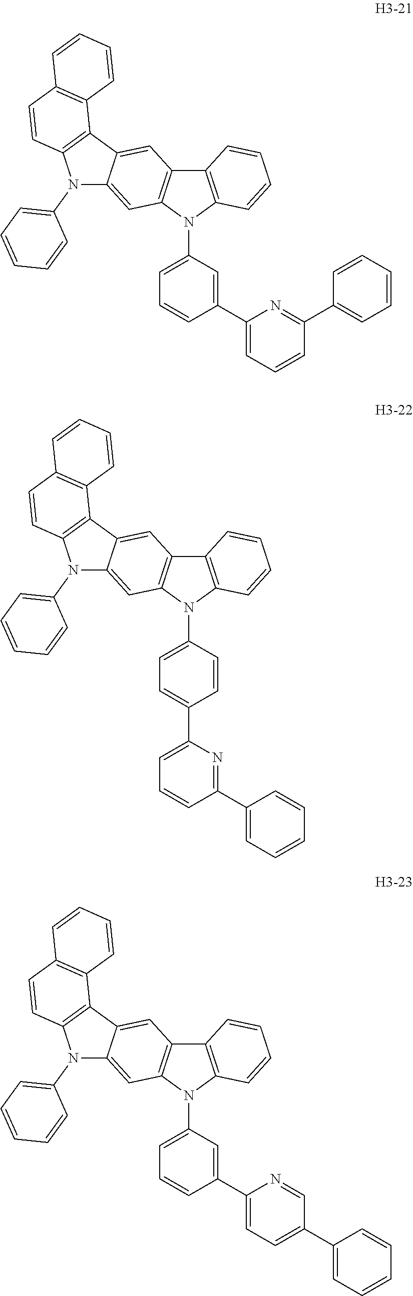 [SMILES: c1ccc(-c2cccc(-c3cccc(-n4c5ccccc5c5cc6c7c8ccccc8ccc7n(-c7ccccc7)c6cc54)c3)n2)cc1 ; c1ccc(-c2cccc(-c3ccc(-n4c5ccccc5c5cc6c7c8ccccc8ccc7n(-c7ccccc7)c6cc54)cc3)n2)cc1 ; c1ccc(-c2ccc(-c3cccc(-n4c5ccccc5c5cc6c7c8ccccc8ccc7n(-c7ccccc7)c6cc54)c3)nc2)cc1]

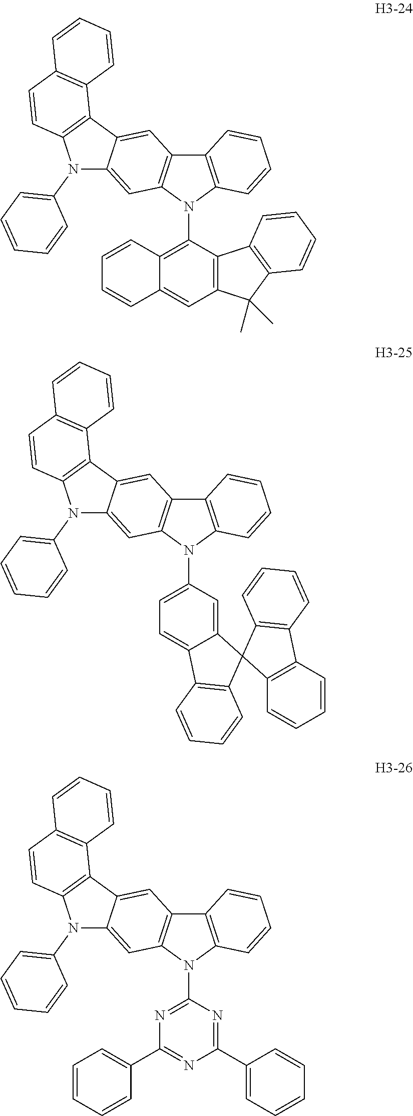 [SMILES: CC1(C)c2ccccc2-c2c1cc1ccccc1c2-n1c2ccccc2c2cc3c4c5ccccc5ccc4n(-c4ccccc4)c3cc21 ; c1ccc(-n2c3cc4c(cc3c3c5ccccc5ccc32)c2ccccc2n4-c2ccc3c(c2)C2(c4ccccc4-c4ccccc42)c2ccccc2-3)cc1 ; c1ccc(-c2nc(-c3ccccc3)nc(-n3c4ccccc4c4cc5c6c7ccccc7ccc6n(-c6ccccc6)c5cc43)n2)cc1]

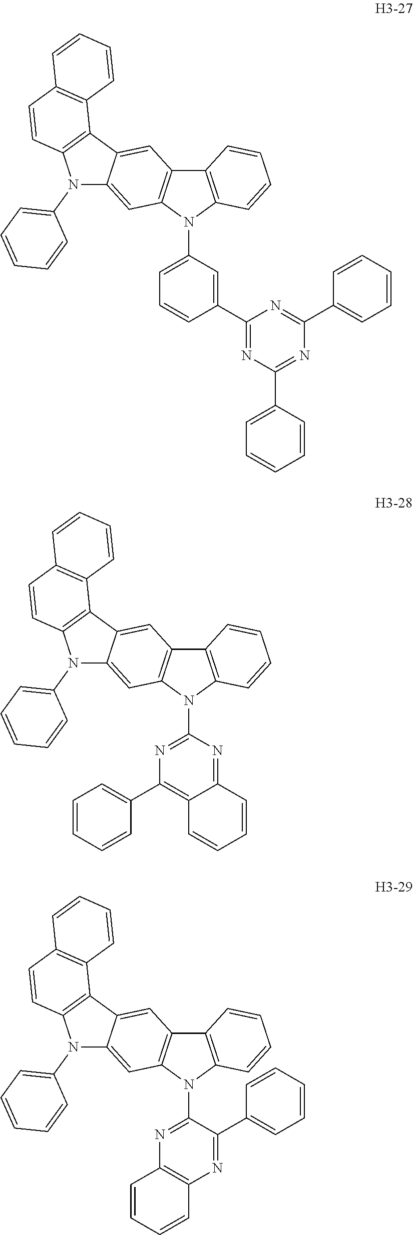 [SMILES: c1ccc(-c2nc(-c3ccccc3)nc(-c3cccc(-n4c5ccccc5c5cc6c7c8ccccc8ccc7n(-c7ccccc7)c6cc54)c3)n2)cc1 ; c1ccc(-c2nc(-n3c4ccccc4c4cc5c6c7ccccc7ccc6n(-c6ccccc6)c5cc43)nc3ccccc23)cc1 ; c1ccc(-c2nc3ccccc3nc2-n2c3ccccc3c3cc4c5c6ccccc6ccc5n(-c5ccccc5)c4cc32)cc1]

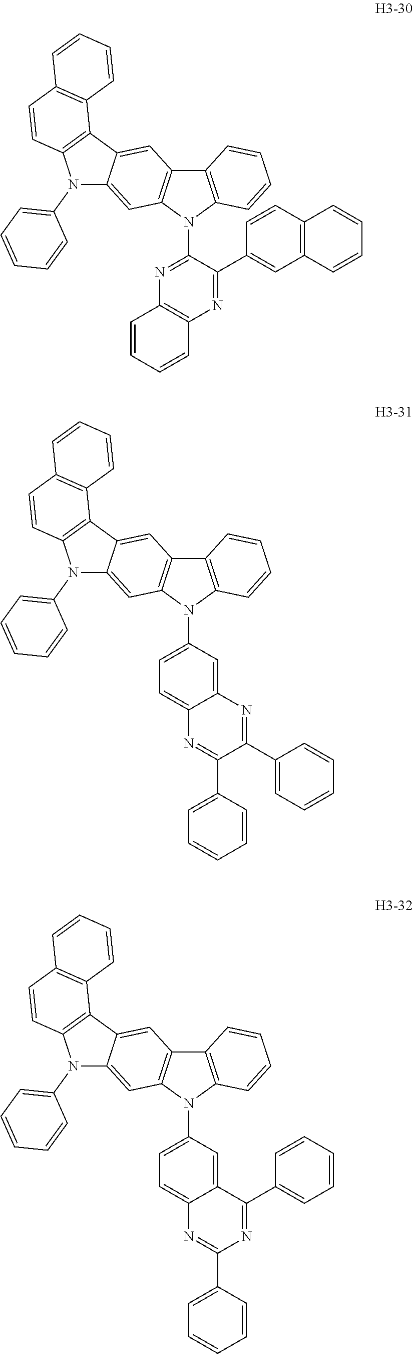 [SMILES: c1ccc(-n2c3cc4c(cc3c3c5ccccc5ccc32)c2ccccc2n4-c2nc3ccccc3nc2-c2ccc3ccccc3c2)cc1 ; c1ccc(-c2nc3ccc(-n4c5ccccc5c5cc6c7c8ccccc8ccc7n(-c7ccccc7)c6cc54)cc3nc2-c2ccccc2)cc1 ; c1ccc(-c2nc(-c3ccccc3)c3cc(-n4c5ccccc5c5cc6c7c8ccccc8ccc7n(-c7ccccc7)c6cc54)ccc3n2)cc1]

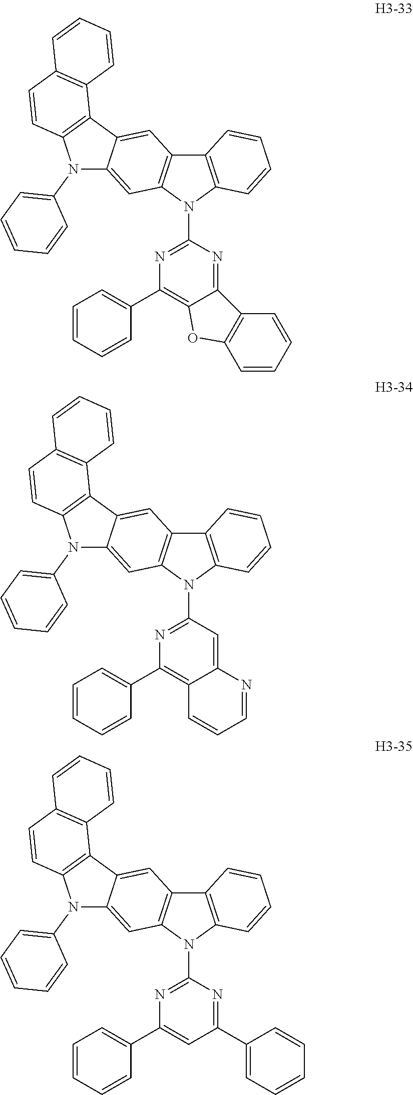

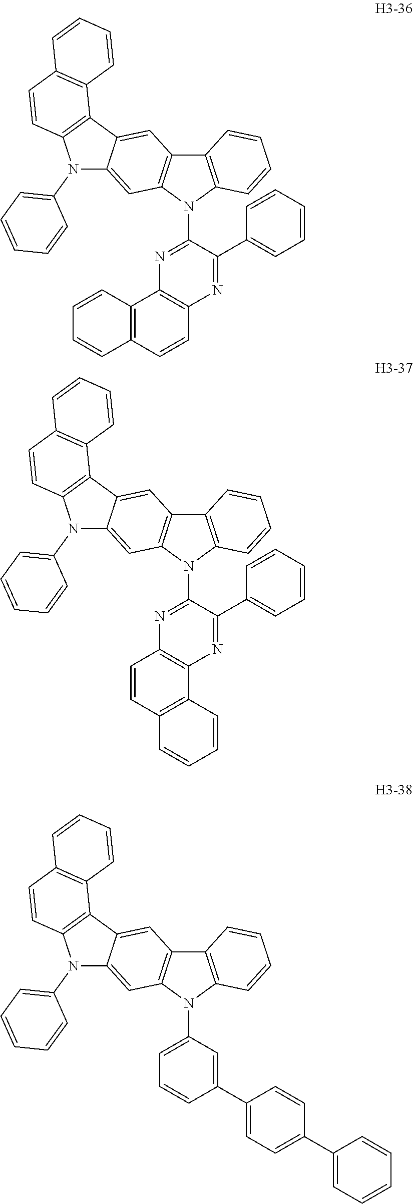

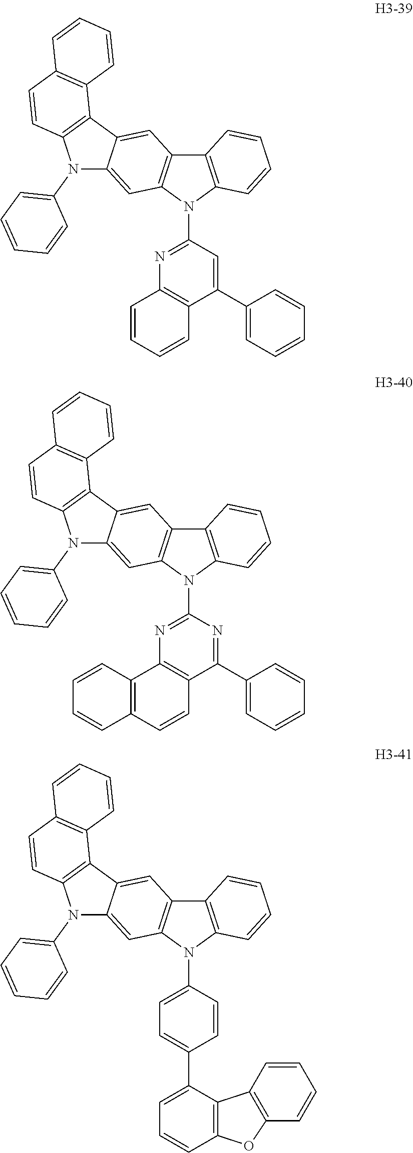

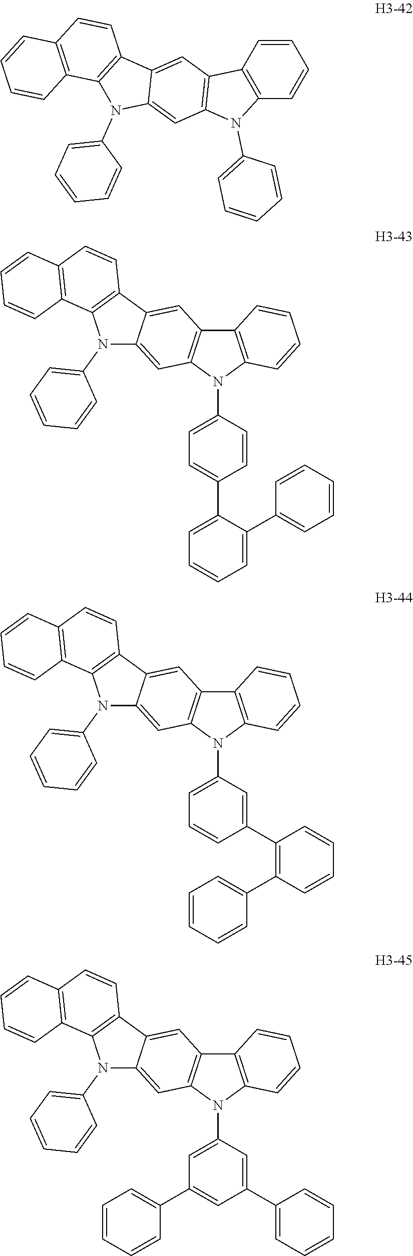

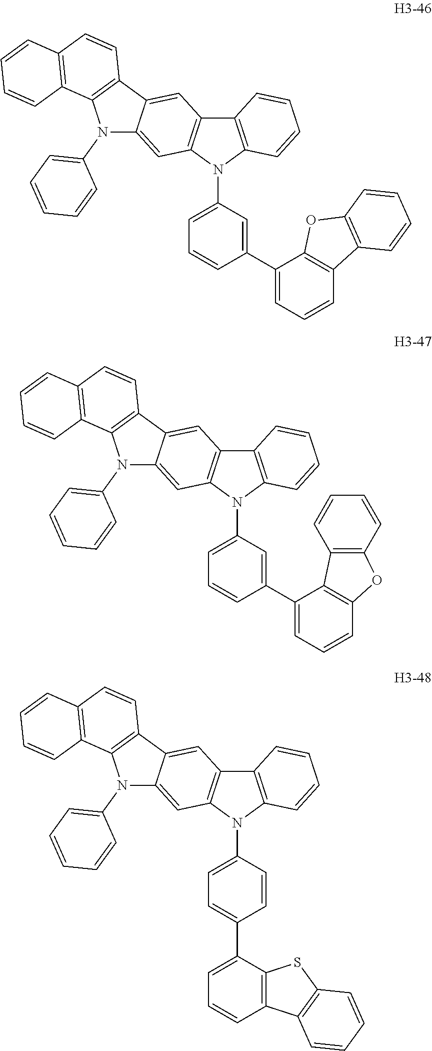 [SMILES: c1ccc(-n2c3cc4c(cc3c3ccc5ccccc5c32)c2ccccc2n4-c2cccc(-c3cccc4c3oc3ccccc34)c2)cc1 ; c1ccc(-n2c3cc4c(cc3c3ccc5ccccc5c32)c2ccccc2n4-c2cccc(-c3cccc4oc5ccccc5c34)c2)cc1 ; c1ccc(-n2c3cc4c(cc3c3ccc5ccccc5c32)c2ccccc2n4-c2ccc(-c3cccc4c3sc3ccccc34)cc2)cc1]

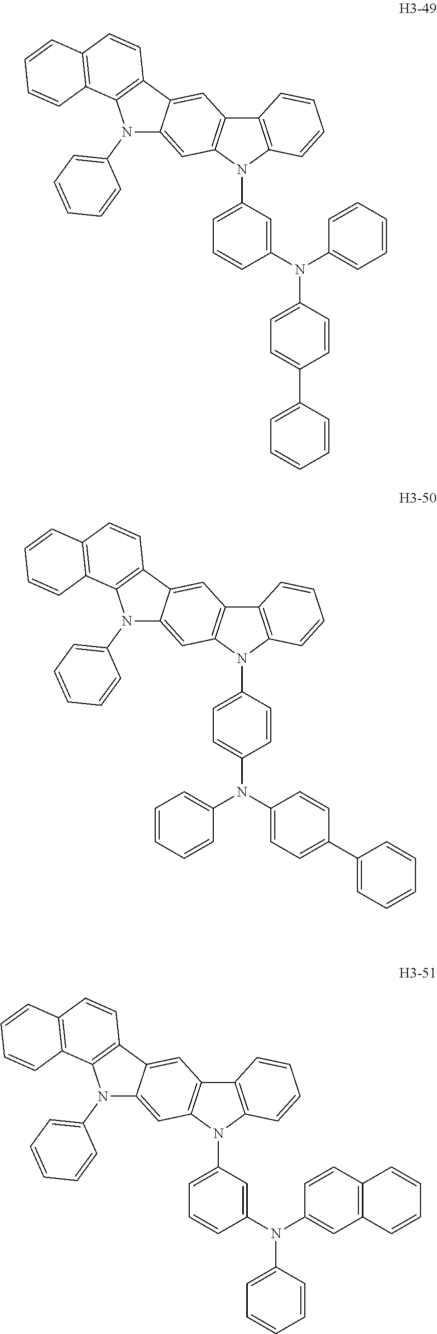 [SMILES: c1ccc(-c2ccc(N(c3ccccc3)c3cccc(-n4c5ccccc5c5cc6c7ccc8ccccc8c7n(-c7ccccc7)c6cc54)c3)cc2)cc1 ; c1ccc(-c2ccc(N(c3ccccc3)c3ccc(-n4c5ccccc5c5cc6c7ccc8ccccc8c7n(-c7ccccc7)c6cc54)cc3)cc2)cc1 ; c1ccc(N(c2cccc(-n3c4ccccc4c4cc5c6ccc7ccccc7c6n(-c6ccccc6)c5cc43)c2)c2ccc3ccccc3c2)cc1]

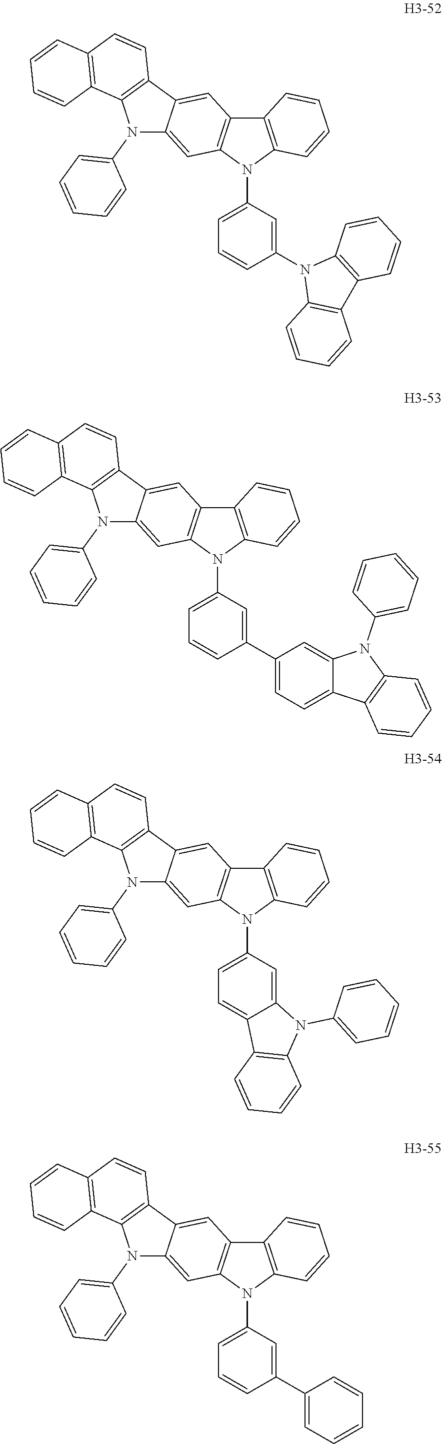 [SMILES: c1ccc(-n2c3cc4c(cc3c3ccc5ccccc5c32)c2ccccc2n4-c2cccc(-n3c4ccccc4c4ccccc43)c2)cc1 ; c1ccc(-n2c3ccccc3c3ccc(-c4cccc(-n5c6ccccc6c6cc7c8ccc9ccccc9c8n(-c8ccccc8)c7cc65)c4)cc32)cc1 ; c1ccc(-n2c3ccccc3c3ccc(-n4c5ccccc5c5cc6c7ccc8ccccc8c7n(-c7ccccc7)c6cc54)cc32)cc1 ; c1ccc(-c2cccc(-n3c4ccccc4c4cc5c6ccc7ccccc7c6n(-c6ccccc6)c5cc43)c2)cc1]

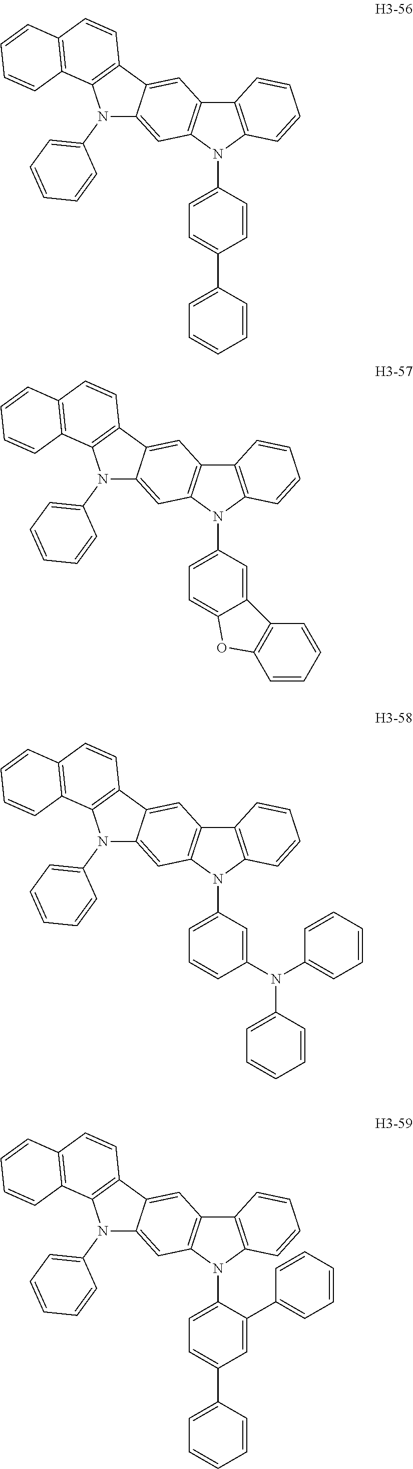 [SMILES: c1ccc(-c2ccc(-n3c4ccccc4c4cc5c6ccc7ccccc7c6n(-c6ccccc6)c5cc43)cc2)cc1 ; c1ccc(-n2c3cc4c(cc3c3ccc5ccccc5c32)c2ccccc2n4-c2ccc3oc4ccccc4c3c2)cc1 ; c1ccc(N(c2ccccc2)c2cccc(-n3c4ccccc4c4cc5c6ccc7ccccc7c6n(-c6ccccc6)c5cc43)c2)cc1 ; c1ccc(-c2ccc(-n3c4ccccc4c4cc5c6ccc7ccccc7c6n(-c6ccccc6)c5cc43)c(-c3ccccc3)c2)cc1]

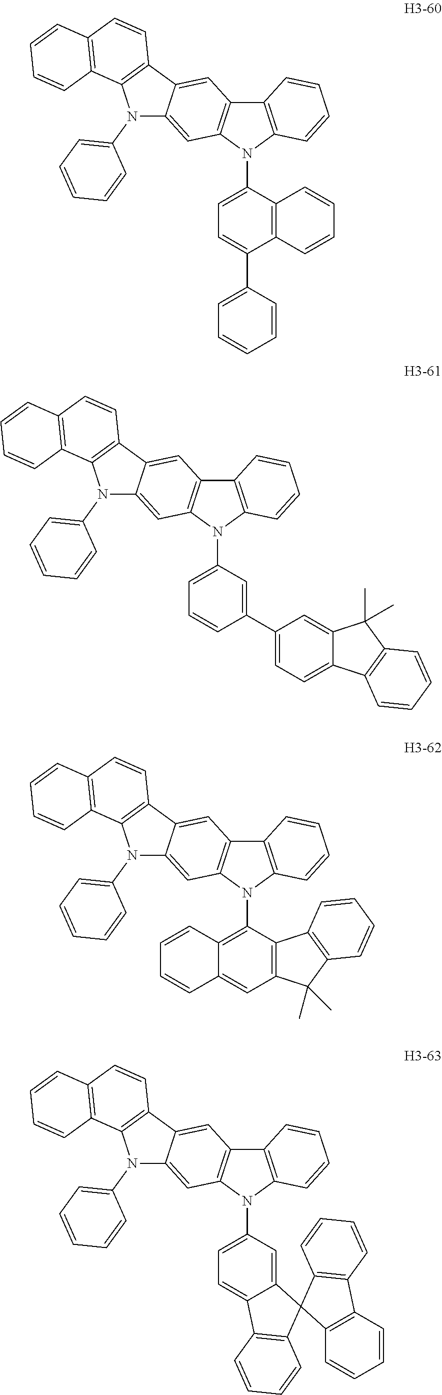 [SMILES: c1ccc(-c2ccc(-n3c4ccccc4c4cc5c6ccc7ccccc7c6n(-c6ccccc6)c5cc43)c3ccccc23)cc1 ; CC1(C)c2ccccc2-c2ccc(-c3cccc(-n4c5ccccc5c5cc6c7ccc8ccccc8c7n(-c7ccccc7)c6cc54)c3)cc21 ; CC1(C)c2ccccc2-c2c1cc1ccccc1c2-n1c2ccccc2c2cc3c4ccc5ccccc5c4n(-c4ccccc4)c3cc21 ; c1ccc(-n2c3cc4c(cc3c3ccc5ccccc5c32)c2ccccc2n4-c2ccc3c(c2)C2(c4ccccc4-c4ccccc42)c2ccccc2-3)cc1]

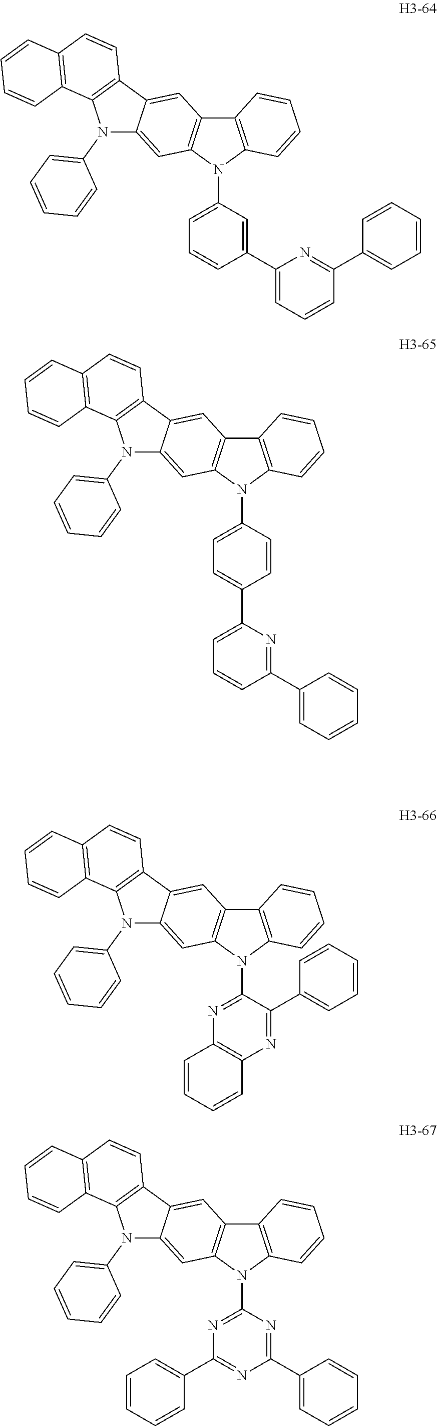 [SMILES: c1ccc(-c2cccc(-c3cccc(-n4c5ccccc5c5cc6c7ccc8ccccc8c7n(-c7ccccc7)c6cc54)c3)n2)cc1 ; c1ccc(-c2cccc(-c3ccc(-n4c5ccccc5c5cc6c7ccc8ccccc8c7n(-c7ccccc7)c6cc54)cc3)n2)cc1 ; c1ccc(-c2nc3ccccc3nc2-n2c3ccccc3c3cc4c5ccc6ccccc6c5n(-c5ccccc5)c4cc32)cc1 ; c1ccc(-c2nc(-c3ccccc3)nc(-n3c4ccccc4c4cc5c6ccc7ccccc7c6n(-c6ccccc6)c5cc43)n2)cc1]

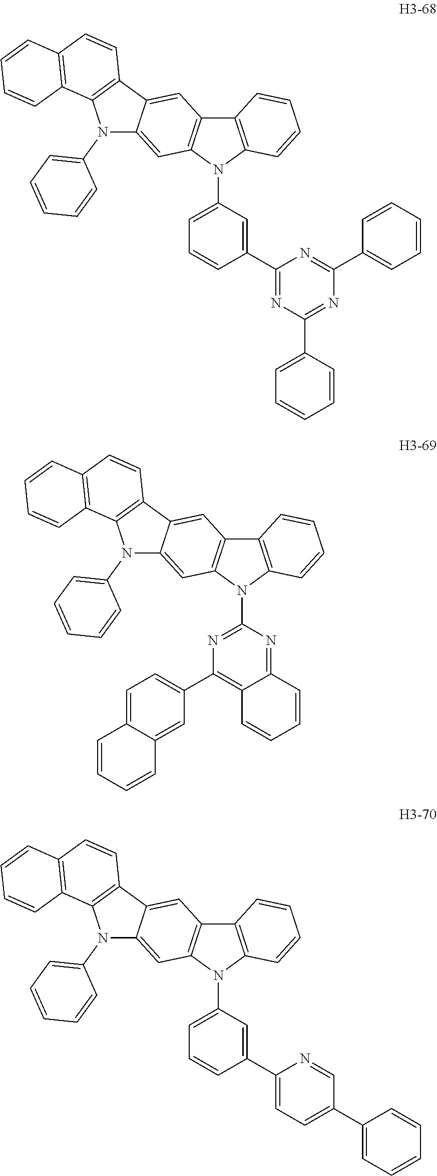 [SMILES: c1ccc(-c2nc(-c3ccccc3)nc(-c3cccc(-n4c5ccccc5c5cc6c7ccc8ccccc8c7n(-c7ccccc7)c6cc54)c3)n2)cc1 ; c1ccc(-n2c3cc4c(cc3c3ccc5ccccc5c32)c2ccccc2n4-c2nc(-c3ccc4ccccc4c3)c3ccccc3n2)cc1 ; c1ccc(-c2ccc(-c3cccc(-n4c5ccccc5c5cc6c7ccc8ccccc8c7n(-c7ccccc7)c6cc54)c3)nc2)cc1]

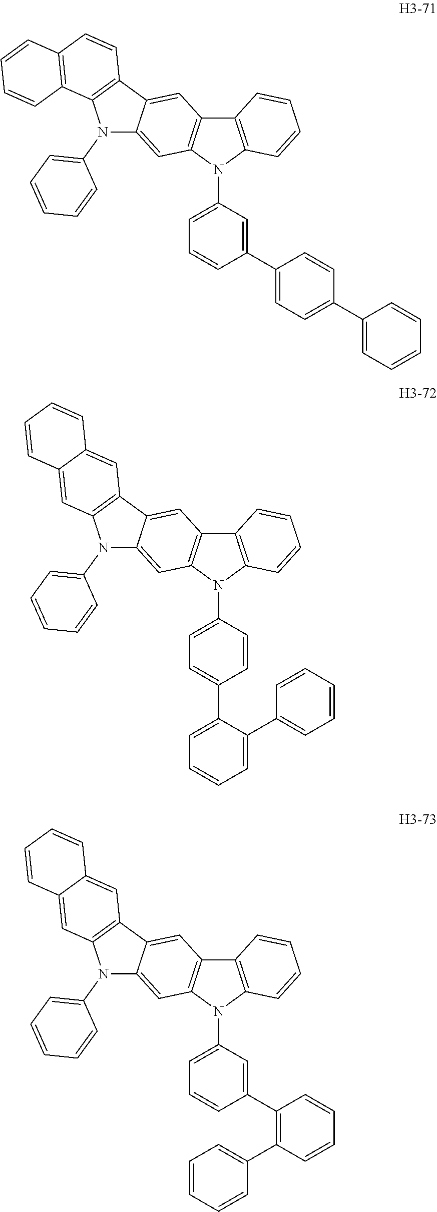 [SMILES: c1ccc(-c2ccc(-c3cccc(-n4c5ccccc5c5cc6c7ccc8ccccc8c7n(-c7ccccc7)c6cc54)c3)cc2)cc1 ; c1ccc(-c2ccccc2-c2ccc(-n3c4ccccc4c4cc5c6cc7ccccc7cc6n(-c6ccccc6)c5cc43)cc2)cc1 ; c1ccc(-c2ccccc2-c2cccc(-n3c4ccccc4c4cc5c6cc7ccccc7cc6n(-c6ccccc6)c5cc43)c2)cc1]

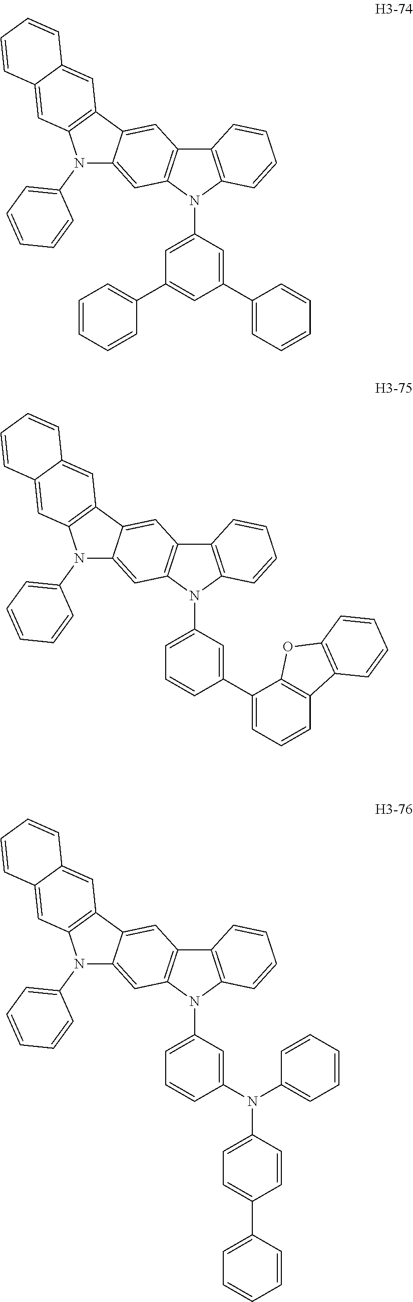 [SMILES: c1ccc(-c2cc(-c3ccccc3)cc(-n3c4ccccc4c4cc5c6cc7ccccc7cc6n(-c6ccccc6)c5cc43)c2)cc1 ; c1ccc(-n2c3cc4ccccc4cc3c3cc4c5ccccc5n(-c5cccc(-c6cccc7c6oc6ccccc67)c5)c4cc32)cc1 ; c1ccc(-c2ccc(N(c3ccccc3)c3cccc(-n4c5ccccc5c5cc6c7cc8ccccc8cc7n(-c7ccccc7)c6cc54)c3)cc2)cc1]

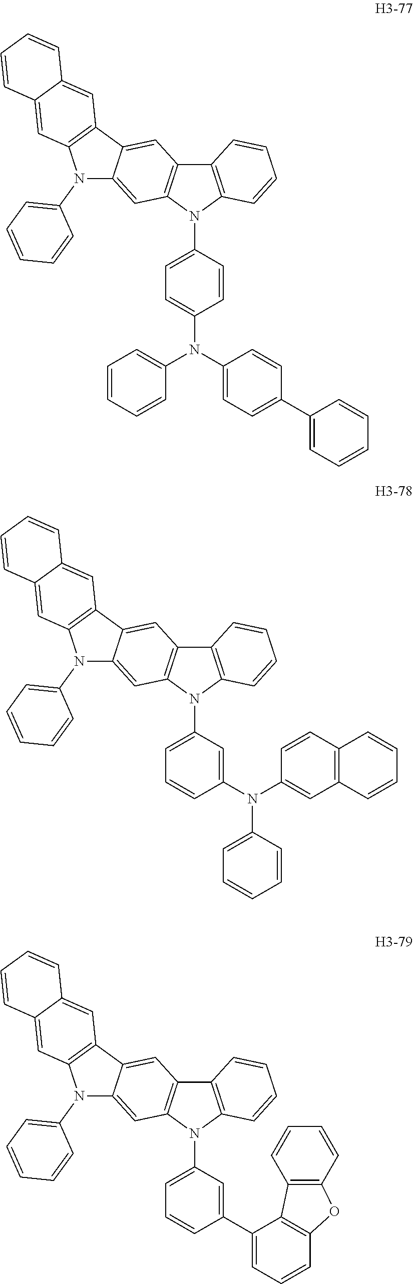 [SMILES: c1ccc(-c2ccc(N(c3ccccc3)c3ccc(-n4c5ccccc5c5cc6c7cc8ccccc8cc7n(-c7ccccc7)c6cc54)cc3)cc2)cc1 ; c1ccc(N(c2cccc(-n3c4ccccc4c4cc5c6cc7ccccc7cc6n(-c6ccccc6)c5cc43)c2)c2ccc3ccccc3c2)cc1 ; c1ccc(-n2c3cc4ccccc4cc3c3cc4c5ccccc5n(-c5cccc(-c6cccc7oc8ccccc8c67)c5)c4cc32)cc1]

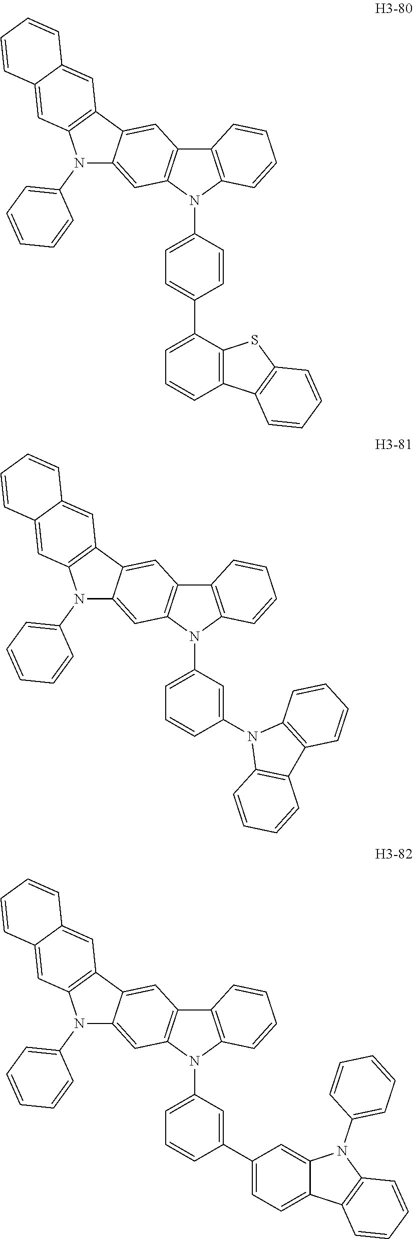 [SMILES: c1ccc(-n2c3cc4ccccc4cc3c3cc4c5ccccc5n(-c5ccc(-c6cccc7c6sc6ccccc67)cc5)c4cc32)cc1 ; c1ccc(-n2c3cc4ccccc4cc3c3cc4c5ccccc5n(-c5cccc(-n6c7ccccc7c7ccccc76)c5)c4cc32)cc1 ; c1ccc(-n2c3ccccc3c3ccc(-c4cccc(-n5c6ccccc6c6cc7c8cc9ccccc9cc8n(-c8ccccc8)c7cc65)c4)cc32)cc1]

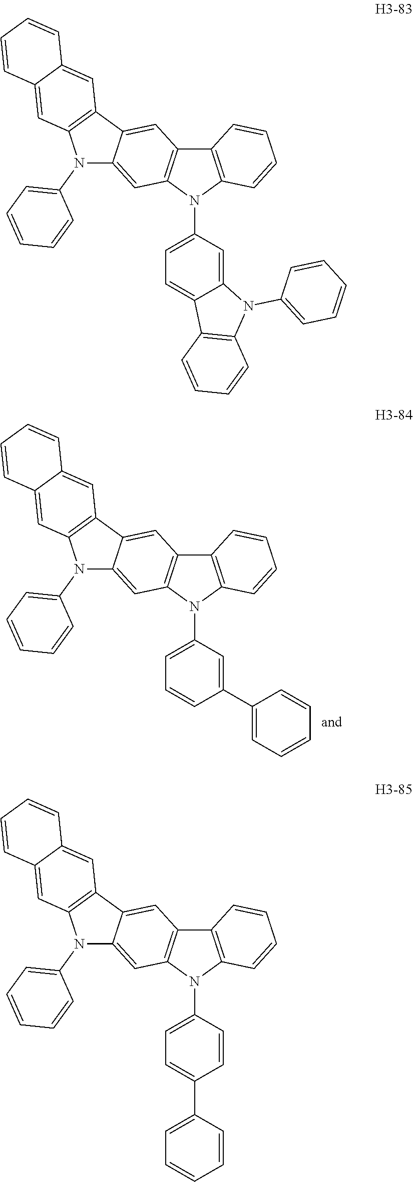 [SMILES: c1ccc(-n2c3ccccc3c3ccc(-n4c5ccccc5c5cc6c7cc8ccccc8cc7n(-c7ccccc7)c6cc54)cc32)cc1 ; c1ccc(-c2cccc(-n3c4ccccc4c4cc5c6cc7ccccc7cc6n(-c6ccccc6)c5cc43)c2)cc1 ; c1ccc(-c2ccc(-n3c4ccccc4c4cc5c6cc7ccccc7cc6n(-c6ccccc6)c5cc43)cc2)cc1]

18. A plurality of host materials comprising a first host material and a second host material, wherein the first host material comprises an organic electroluminescent compound according to claim 1 and the second host material comprises an organic electroluminescent compound represented by the following formula 5:
T-Lₚ-Arₚ (5)
wherein,
Arₚ represents a substituted or unsubstituted (C6-C30)aryl, a substituted or unsubstituted (3- to 30-membered)heteroaryl, or 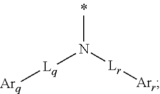
Ar_{q} and Arᵣ each independently represent a substituted or unsubstituted (C6-C30)aryl, a substituted or unsubstituted (3- to 30-membered)heteroaryl, or a substituted or unsubstituted (C3-C30)cycloalkyl;
Lₚ to Lᵣ each independently represent a single bond, a substituted or unsubstituted (C6-C30)arylene, or a substituted or unsubstituted (3- to 30-membered)heteroarylene; and
T is represented by the following formula 5-1 or 5-2; 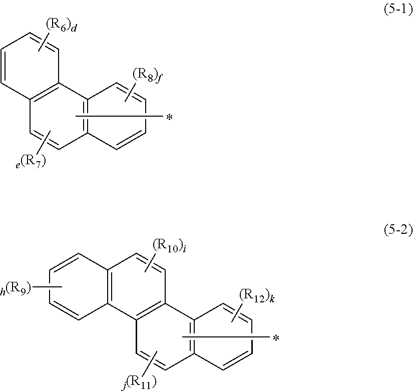
wherein,
R₆ to R₁₂ each independently represent hydrogen, deuterium, halogen, cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring of (C3-C30) aliphatic ring and an (C6-C30) aromatic ring, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted (C1-C30)alkyl(C2-C30)alkenylamino, a substituted or unsubstituted mono- or di-(C6-C30)arylamino, a substituted or unsubstituted (C1-C30)alkyl(C6-C30)arylamino, a substituted or unsubstituted mono- or di-(3- to 30-membered)heteroarylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino;
d, f, h, and k are each independently an integer of 1 to 4, and e, i, and j are each independently an integer of 1 or 2; and
when d to f and h to k are 2 or more, each of R₆ to R₁₁ may be the same or different.

19. The plurality of host materials according to claim 18, wherein the formula 5 is represented by the following formula 5-1-1 or 5-2-1: 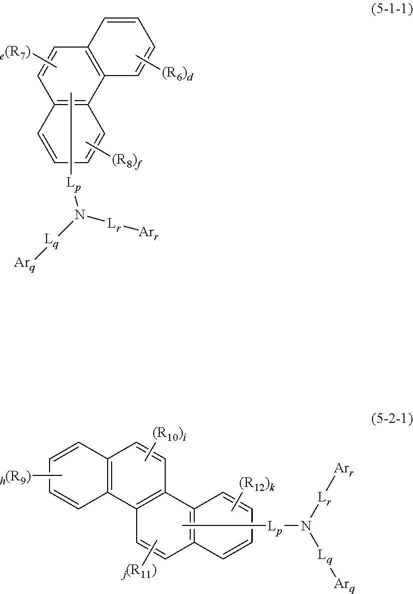
wherein,
R₆ to R₁₂, Lₚ to Lᵣ, Ar_{q}, Arᵣ, d to f, and h to k are as defined in claim 18.

20. The plurality of host materials according to claim 18, wherein the compound represented by formula 5 is selected from the following compounds:

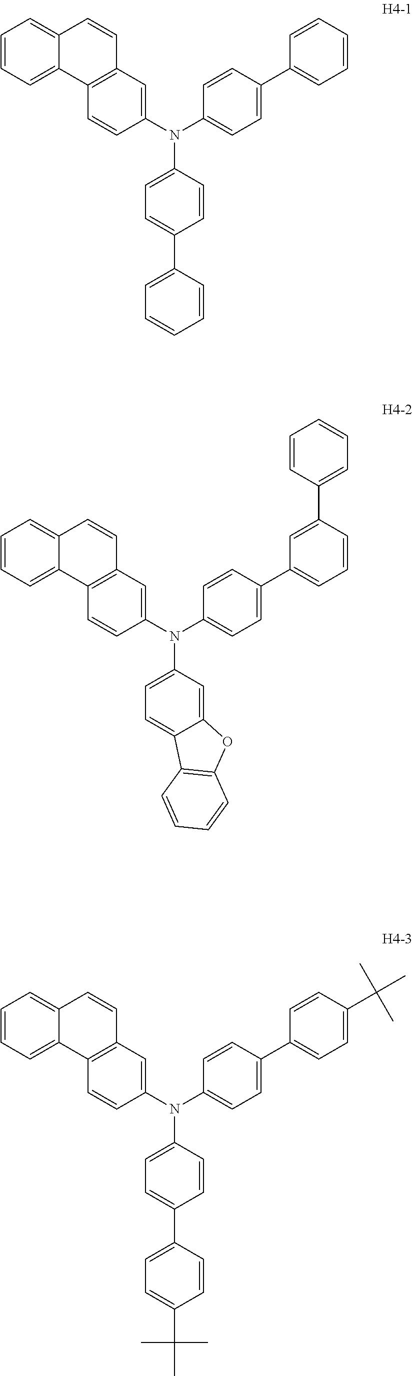 [SMILES: c1ccc(-c2ccc(N(c3ccc(-c4ccccc4)cc3)c3ccc4c(ccc5ccccc54)c3)cc2)cc1 ; c1ccc(-c2cccc(-c3ccc(N(c4ccc5c(ccc6ccccc65)c4)c4ccc5c(c4)oc4ccccc45)cc3)c2)cc1 ; CC(C)(C)c1ccc(-c2ccc(N(c3ccc(-c4ccc(C(C)(C)C)cc4)cc3)c3ccc4c(ccc5ccccc54)c3)cc2)cc1]

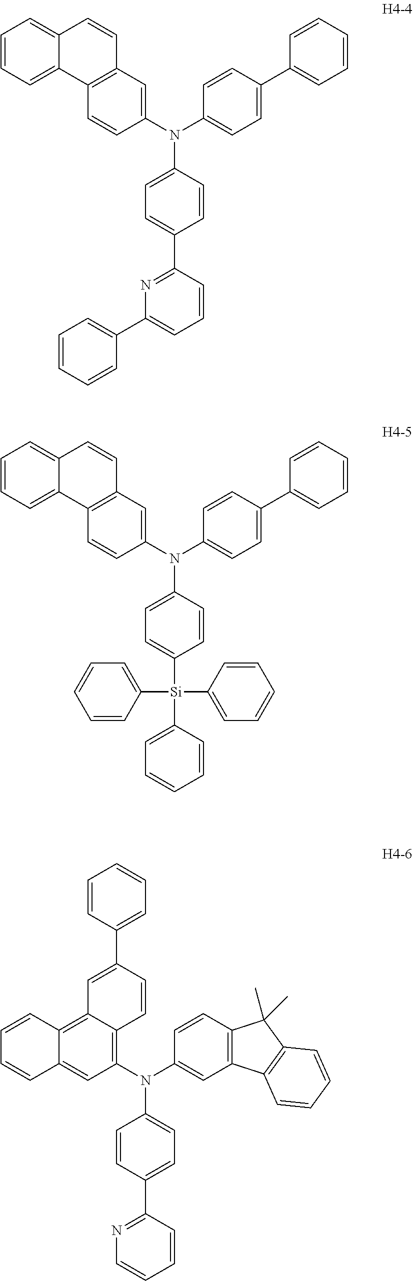 [SMILES: c1ccc(-c2ccc(N(c3ccc(-c4cccc(-c5ccccc5)n4)cc3)c3ccc4c(ccc5ccccc54)c3)cc2)cc1 ; c1ccc(-c2ccc(N(c3ccc([Si](c4ccccc4)(c4ccccc4)c4ccccc4)cc3)c3ccc4c(ccc5ccccc54)c3)cc2)cc1 ; CC1(C)c2ccccc2-c2cc(N(c3ccc(-c4ccccn4)cc3)c3cc4ccccc4c4cc(-c5ccccc5)ccc34)ccc21]

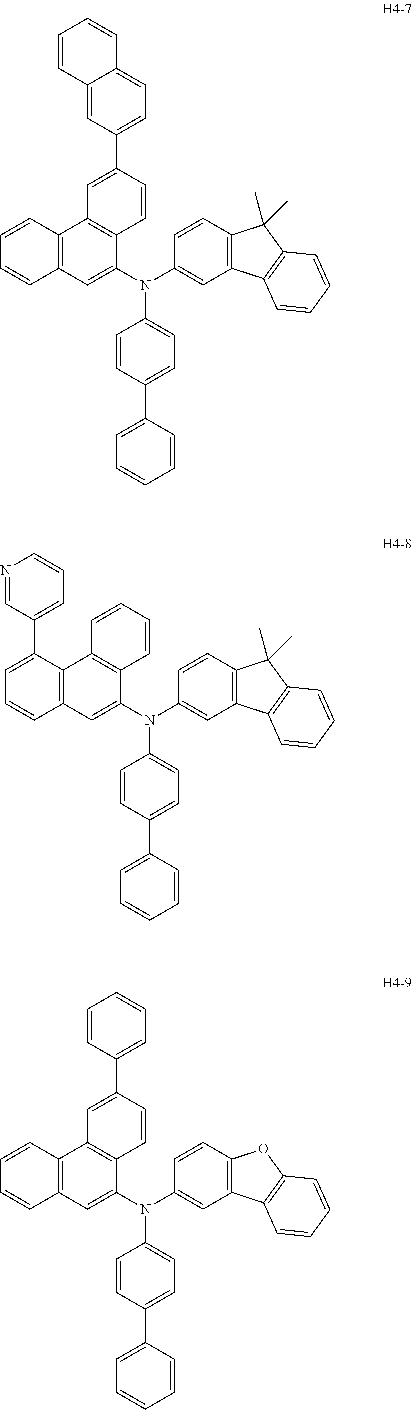 [SMILES: CC1(C)c2ccccc2-c2cc(N(c3ccc(-c4ccccc4)cc3)c3cc4ccccc4c4cc(-c5ccc6ccccc6c5)ccc34)ccc21 ; CC1(C)c2ccccc2-c2cc(N(c3ccc(-c4ccccc4)cc3)c3cc4cccc(-c5cccnc5)c4c4ccccc34)ccc21 ; c1ccc(-c2ccc(N(c3ccc4oc5ccccc5c4c3)c3cc4ccccc4c4cc(-c5ccccc5)ccc34)cc2)cc1]

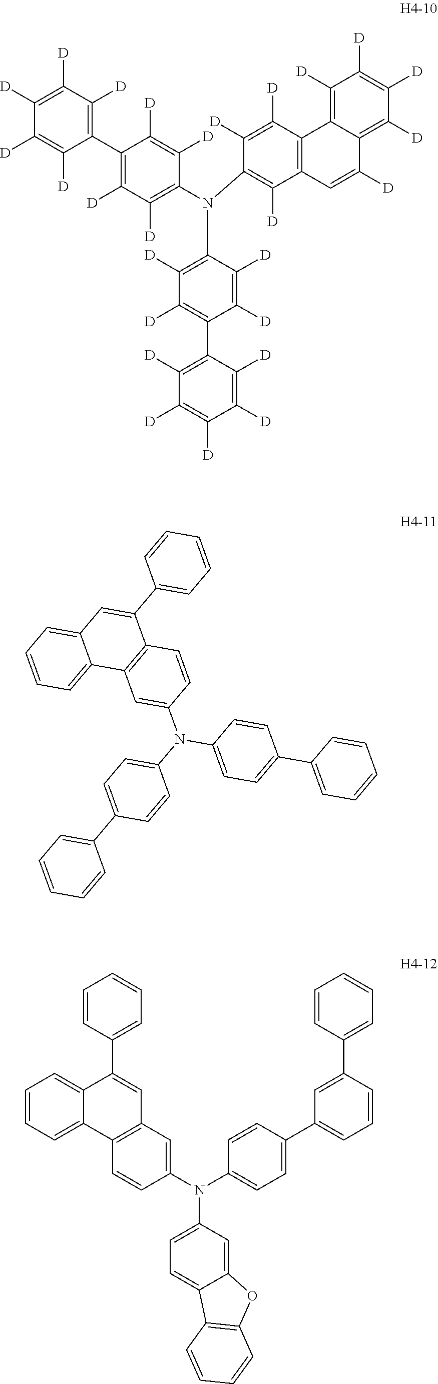 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(cc([2H])c5c([2H])c([2H])c([2H])c([2H])c54)c3[2H])c([2H])c2[2H])c([2H])c1[2H] ; c1ccc(-c2ccc(N(c3ccc(-c4ccccc4)cc3)c3ccc4c(-c5ccccc5)cc5ccccc5c4c3)cc2)cc1 ; c1ccc(-c2cccc(-c3ccc(N(c4ccc5c(c4)cc(-c4ccccc4)c4ccccc45)c4ccc5c(c4)oc4ccccc45)cc3)c2)cc1]

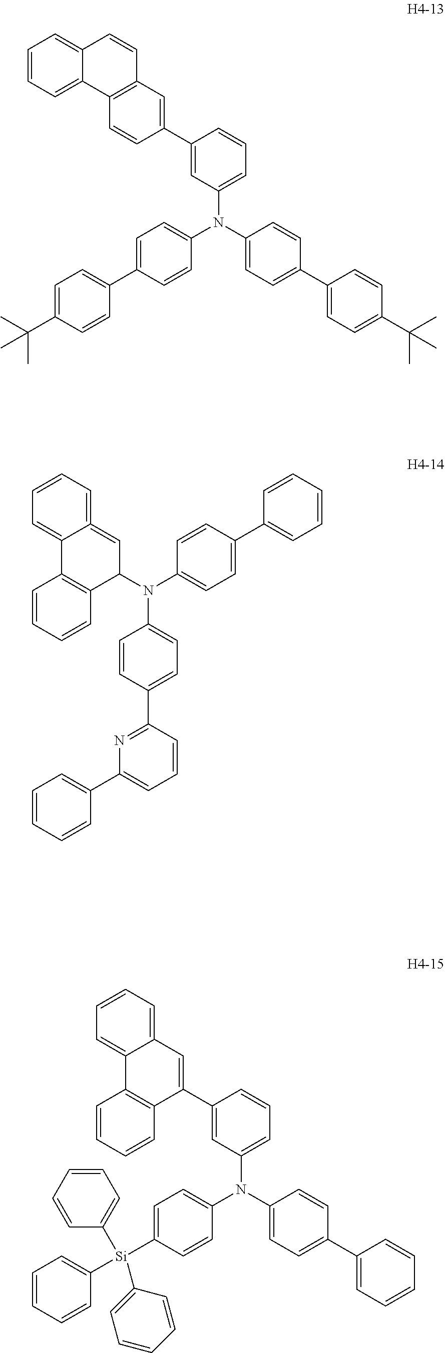

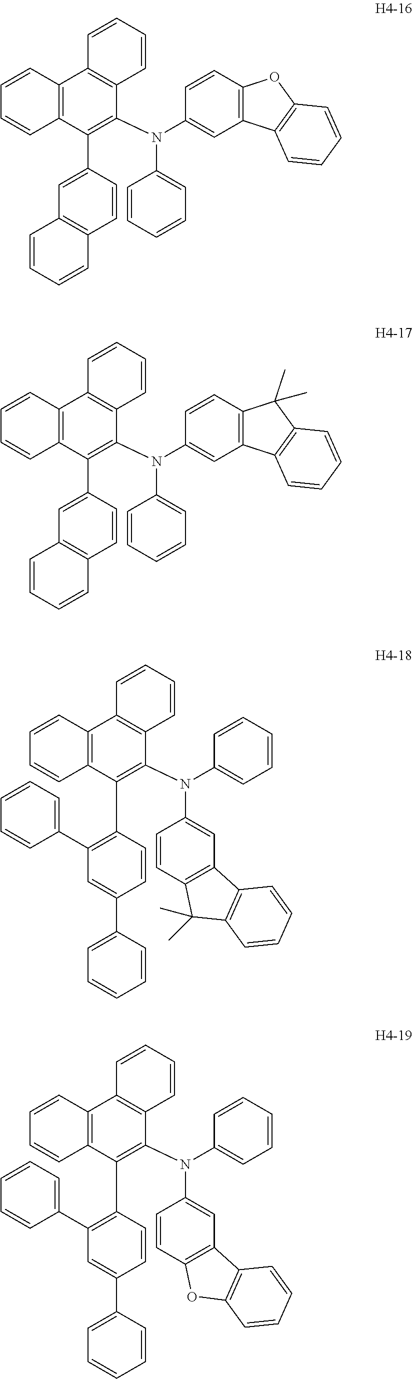 [SMILES: c1ccc(N(c2ccc3oc4ccccc4c3c2)c2c(-c3ccc4ccccc4c3)c3ccccc3c3ccccc23)cc1 ; CC1(C)c2ccccc2-c2cc(N(c3ccccc3)c3c(-c4ccc5ccccc5c4)c4ccccc4c4ccccc34)ccc21 ; CC1(C)c2ccccc2-c2cc(N(c3ccccc3)c3c(-c4ccc(-c5ccccc5)cc4-c4ccccc4)c4ccccc4c4ccccc34)ccc21 ; c1ccc(-c2ccc(-c3c(N(c4ccccc4)c4ccc5oc6ccccc6c5c4)c4ccccc4c4ccccc34)c(-c3ccccc3)c2)cc1]

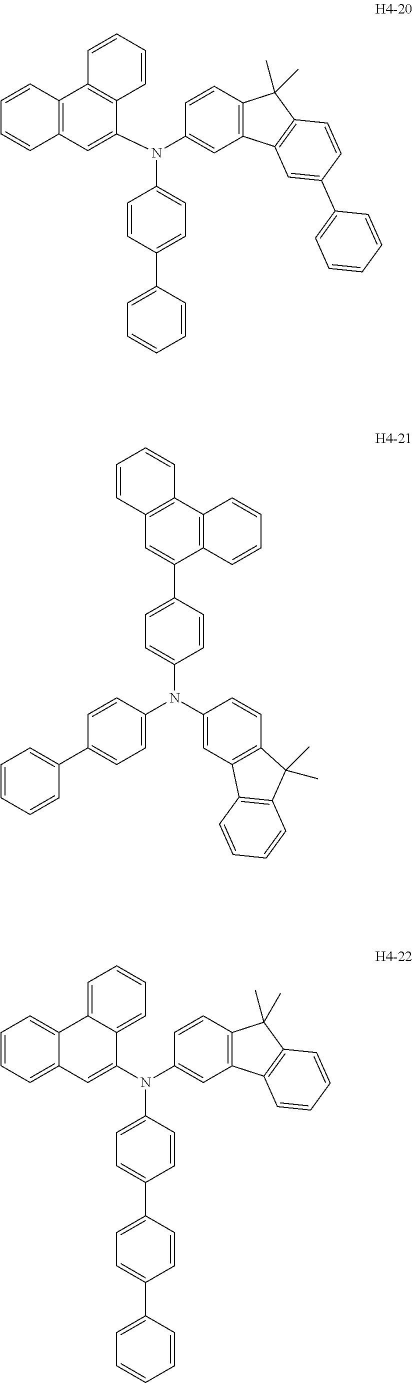 [SMILES: CC1(C)c2ccc(-c3ccccc3)cc2-c2cc(N(c3ccc(-c4ccccc4)cc3)c3cc4ccccc4c4ccccc34)ccc21 ; CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2cc(N(c3ccc(-c4ccc(-c5ccccc5)cc4)cc3)c3cc4ccccc4c4ccccc34)ccc21]

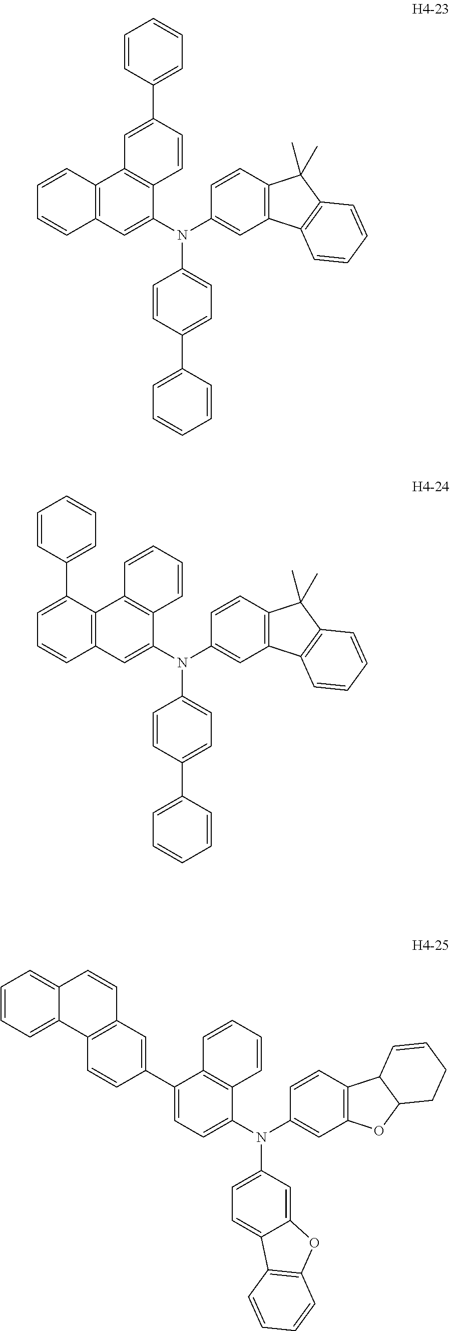 [SMILES: CC1(C)c2ccccc2-c2cc(N(c3ccc(-c4ccccc4)cc3)c3cc4ccccc4c4cc(-c5ccccc5)ccc34)ccc21 ; CC1(C)c2ccccc2-c2cc(N(c3ccc(-c4ccccc4)cc3)c3cc4cccc(-c5ccccc5)c4c4ccccc34)ccc21 ; C1=CC2c3ccc(N(c4ccc5c(c4)oc4ccccc45)c4ccc(-c5ccc6c(ccc7ccccc76)c5)c5ccccc45)cc3OC2CC1]

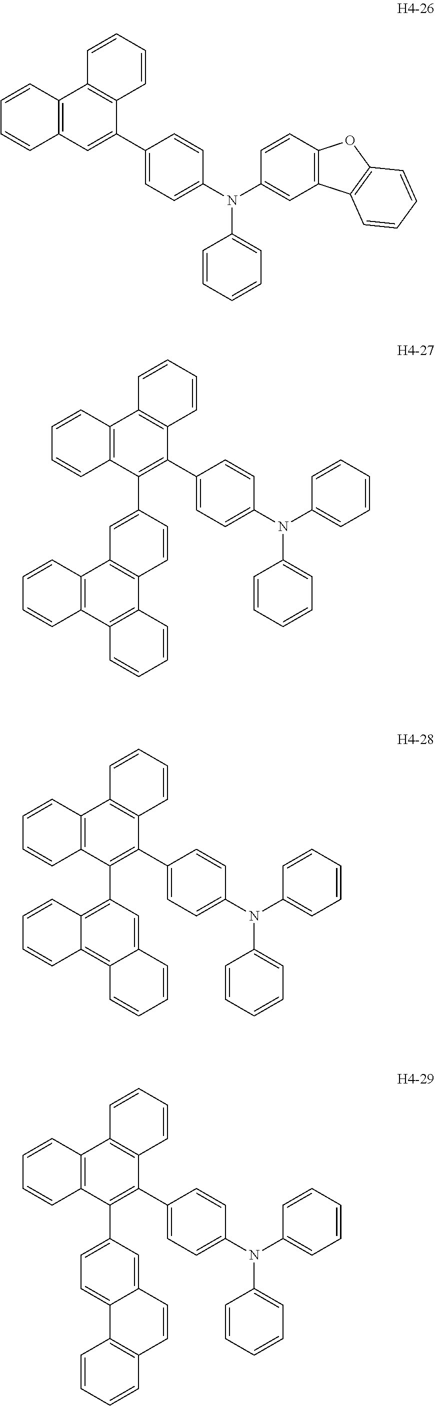 [SMILES: c1ccc(N(c2ccc(-c3cc4ccccc4c4ccccc34)cc2)c2ccc3oc4ccccc4c3c2)cc1 ; c1ccc(N(c2ccccc2)c2ccc(-c3c(-c4ccc5c6ccccc6c6ccccc6c5c4)c4ccccc4c4ccccc34)cc2)cc1 ; c1ccc(N(c2ccccc2)c2ccc(-c3c(-c4cc5ccccc5c5ccccc45)c4ccccc4c4ccccc34)cc2)cc1 ; c1ccc(N(c2ccccc2)c2ccc(-c3c(-c4ccc5c(ccc6ccccc65)c4)c4ccccc4c4ccccc34)cc2)cc1]

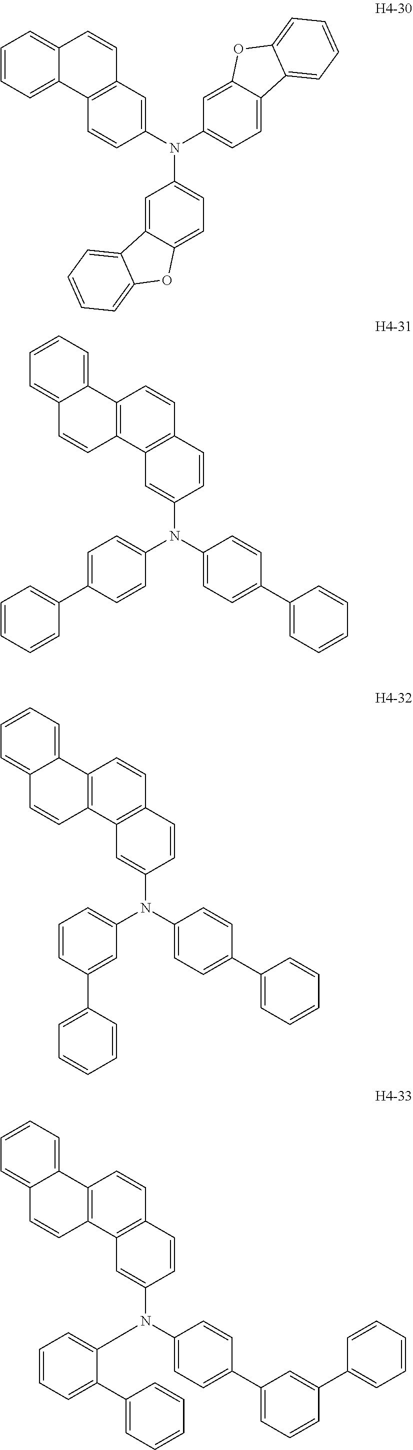

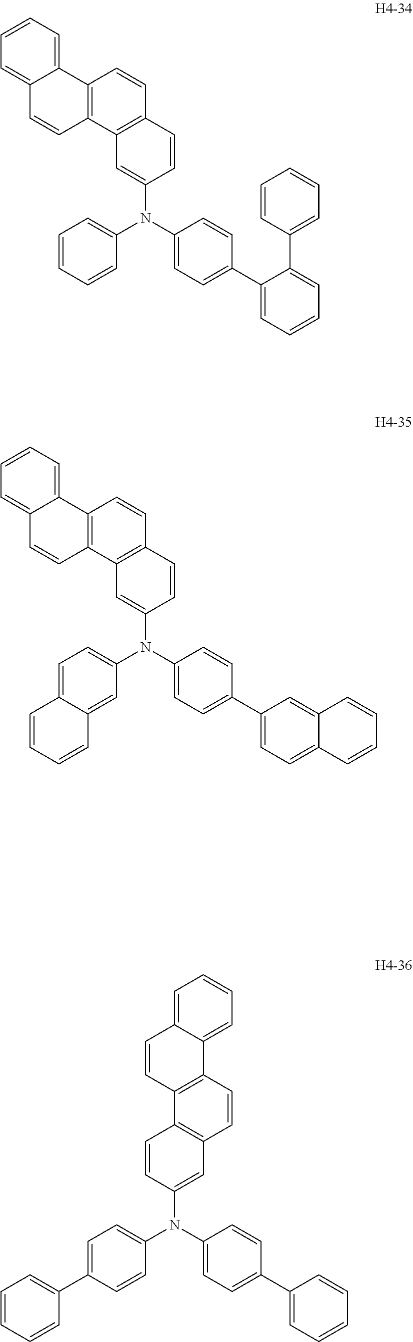 [SMILES: c1ccc(-c2ccccc2-c2ccc(N(c3ccccc3)c3ccc4ccc5c6ccccc6ccc5c4c3)cc2)cc1 ; c1ccc2cc(-c3ccc(N(c4ccc5ccccc5c4)c4ccc5ccc6c7ccccc7ccc6c5c4)cc3)ccc2c1 ; c1ccc(-c2ccc(N(c3ccc(-c4ccccc4)cc3)c3ccc4c(ccc5c6ccccc6ccc45)c3)cc2)cc1]

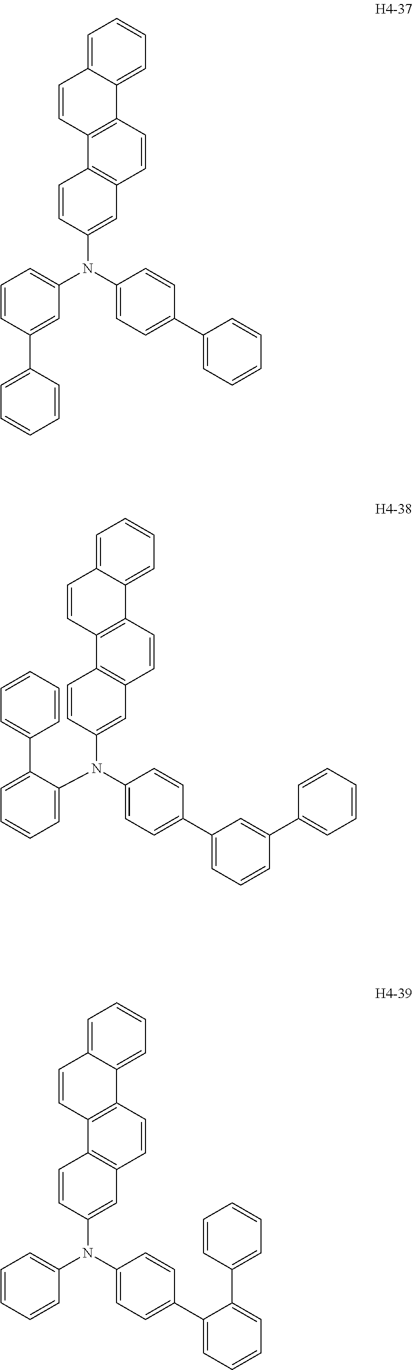 [SMILES: c1ccc(-c2ccc(N(c3cccc(-c4ccccc4)c3)c3ccc4c(ccc5c6ccccc6ccc45)c3)cc2)cc1 ; c1ccc(-c2cccc(-c3ccc(N(c4ccc5c(ccc6c7ccccc7ccc56)c4)c4ccccc4-c4ccccc4)cc3)c2)cc1 ; c1ccc(-c2ccccc2-c2ccc(N(c3ccccc3)c3ccc4c(ccc5c6ccccc6ccc45)c3)cc2)cc1]

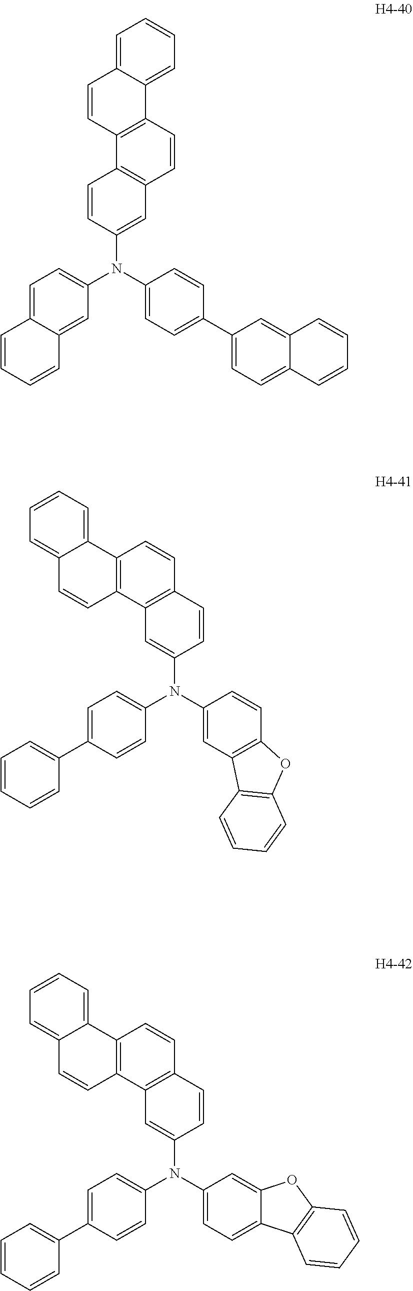 [SMILES: c1ccc2cc(-c3ccc(N(c4ccc5ccccc5c4)c4ccc5c(ccc6c7ccccc7ccc56)c4)cc3)ccc2c1 ; c1ccc(-c2ccc(N(c3ccc4ccc5c6ccccc6ccc5c4c3)c3ccc4oc5ccccc5c4c3)cc2)cc1 ; c1ccc(-c2ccc(N(c3ccc4c(c3)oc3ccccc34)c3ccc4ccc5c6ccccc6ccc5c4c3)cc2)cc1]

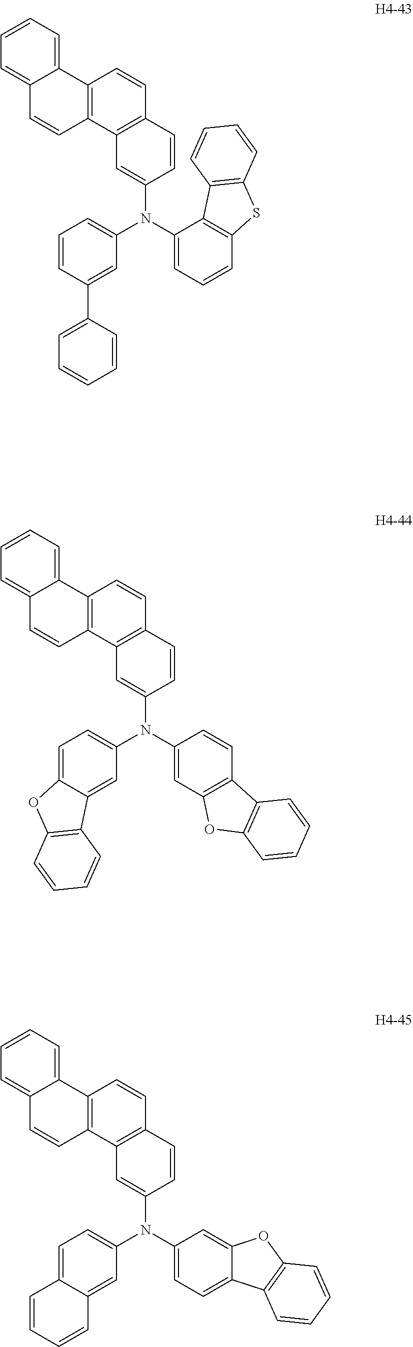 [SMILES: c1ccc(-c2cccc(N(c3ccc4ccc5c6ccccc6ccc5c4c3)c3cccc4sc5ccccc5c34)c2)cc1 ; c1ccc2c(c1)ccc1c3cc(N(c4ccc5c(c4)oc4ccccc45)c4ccc5oc6ccccc6c5c4)ccc3ccc21 ; c1ccc2cc(N(c3ccc4c(c3)oc3ccccc34)c3ccc4ccc5c6ccccc6ccc5c4c3)ccc2c1]

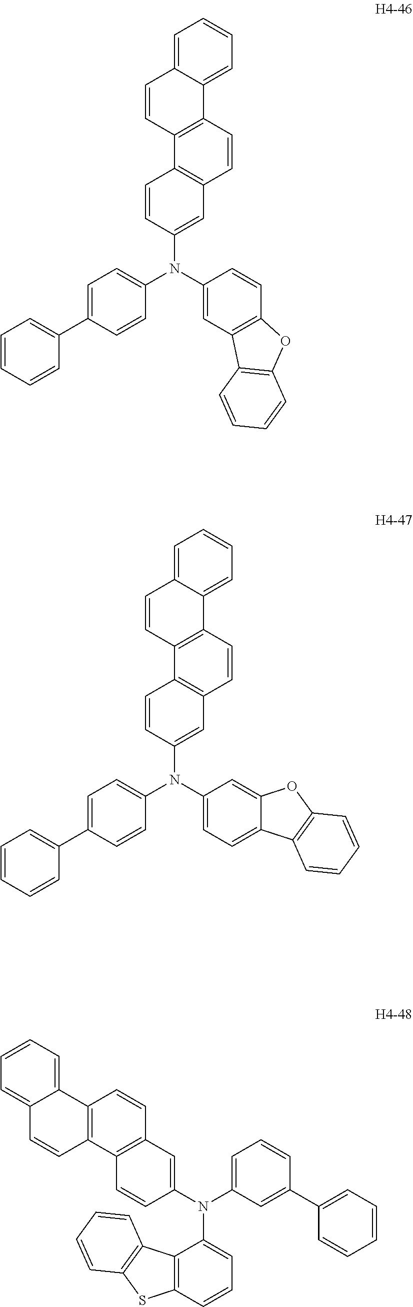 [SMILES: c1ccc(-c2ccc(N(c3ccc4c(ccc5c6ccccc6ccc45)c3)c3ccc4oc5ccccc5c4c3)cc2)cc1 ; c1ccc(-c2ccc(N(c3ccc4c(ccc5c6ccccc6ccc45)c3)c3ccc4c(c3)oc3ccccc34)cc2)cc1 ; c1ccc(-c2cccc(N(c3ccc4c(ccc5c6ccccc6ccc45)c3)c3cccc4sc5ccccc5c34)c2)cc1]

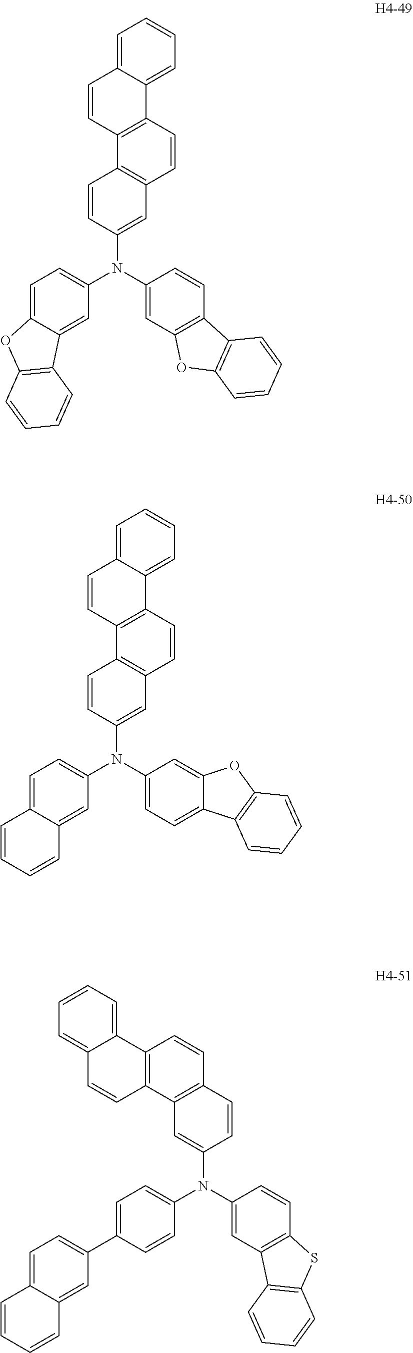 [SMILES: c1ccc2c(c1)ccc1c3ccc(N(c4ccc5c(c4)oc4ccccc45)c4ccc5oc6ccccc6c5c4)cc3ccc21 ; c1ccc2cc(N(c3ccc4c(ccc5c6ccccc6ccc45)c3)c3ccc4c(c3)oc3ccccc34)ccc2c1 ; c1ccc2cc(-c3ccc(N(c4ccc5ccc6c7ccccc7ccc6c5c4)c4ccc5sc6ccccc6c5c4)cc3)ccc2c1]

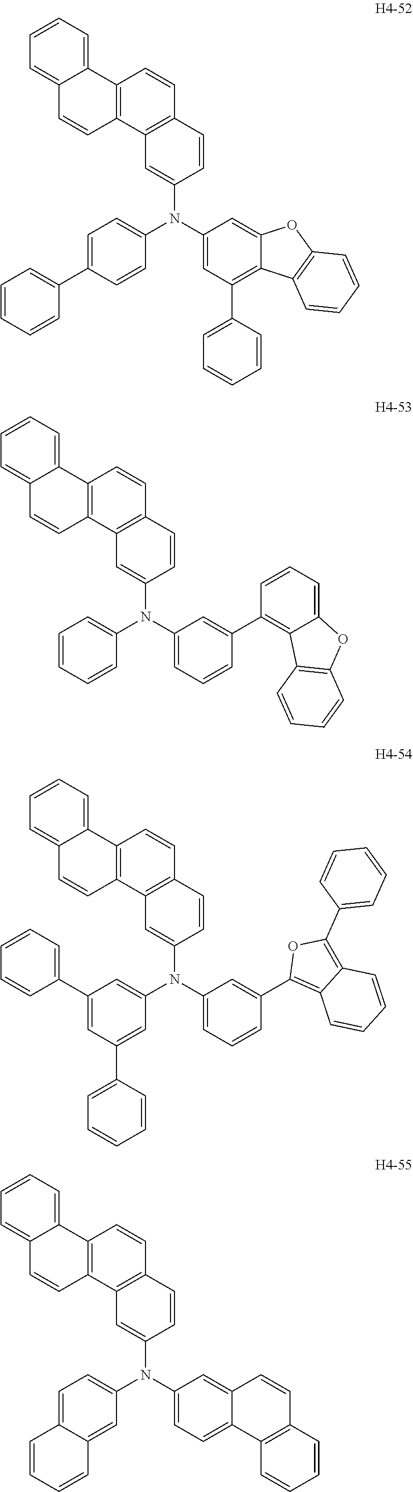 [SMILES: c1ccc(-c2ccc(N(c3cc(-c4ccccc4)c4c(c3)oc3ccccc34)c3ccc4ccc5c6ccccc6ccc5c4c3)cc2)cc1 ; c1ccc(N(c2cccc(-c3cccc4oc5ccccc5c34)c2)c2ccc3ccc4c5ccccc5ccc4c3c2)cc1 ; c1ccc(-c2cc(-c3ccccc3)cc(N(c3cccc(-c4oc(-c5ccccc5)c5ccccc45)c3)c3ccc4ccc5c6ccccc6ccc5c4c3)c2)cc1 ; c1ccc2cc(N(c3ccc4c(ccc5ccccc54)c3)c3ccc4ccc5c6ccccc6ccc5c4c3)ccc2c1]

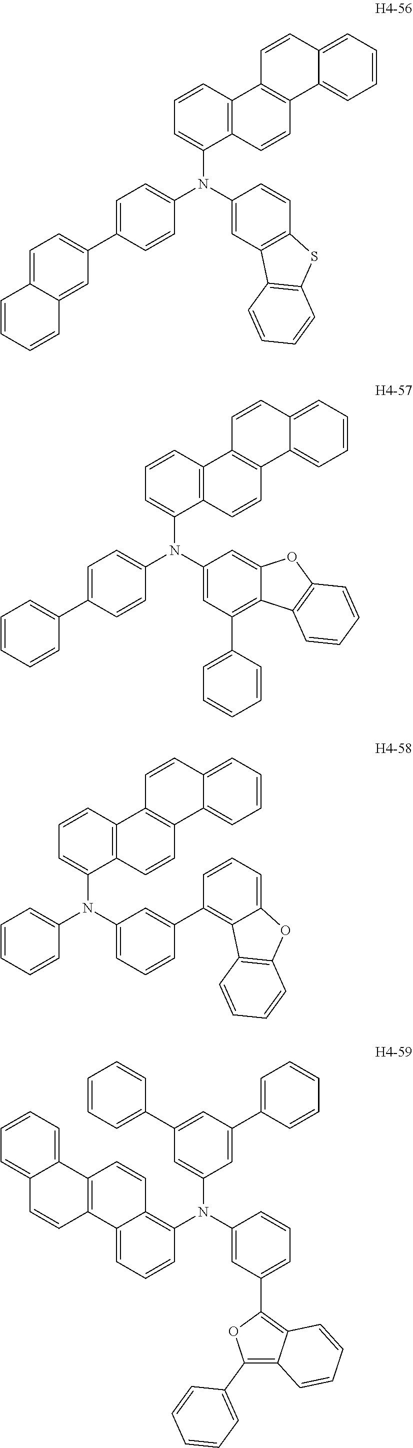

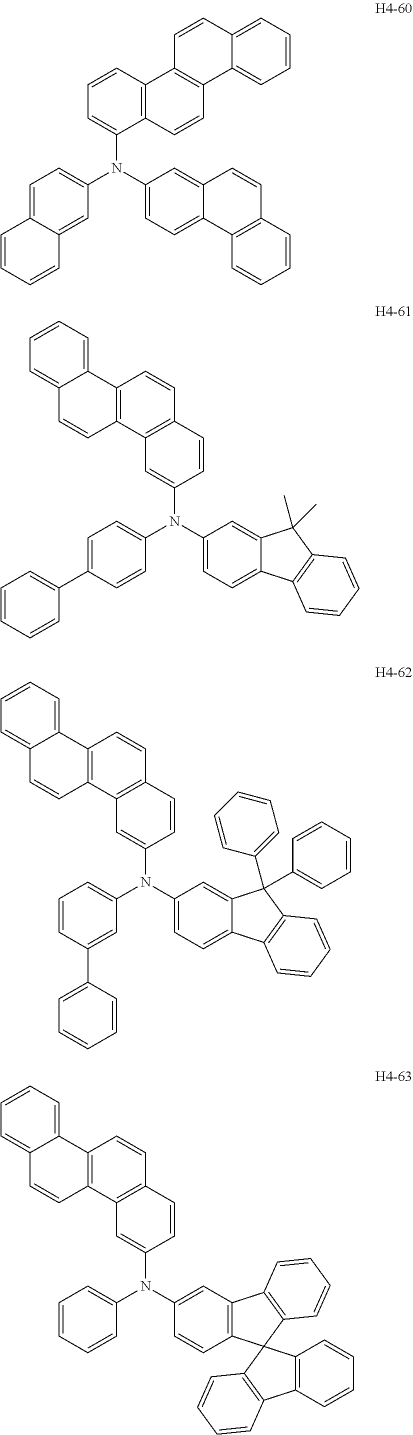

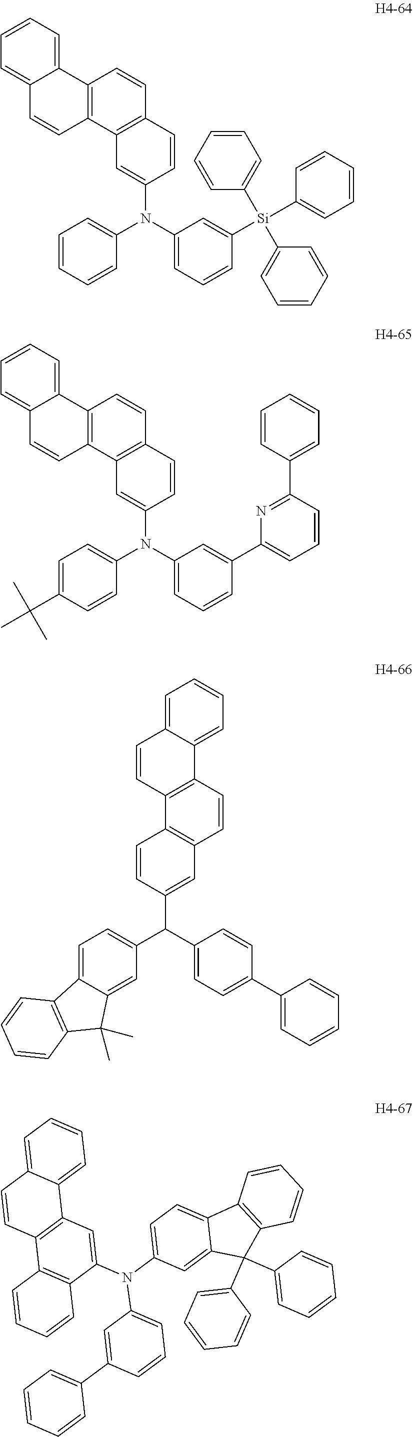

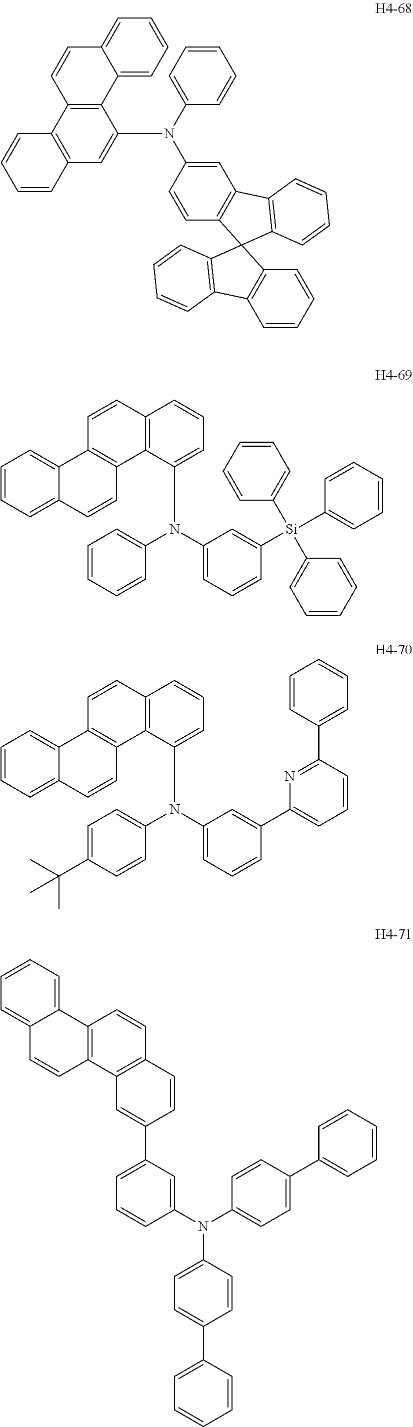

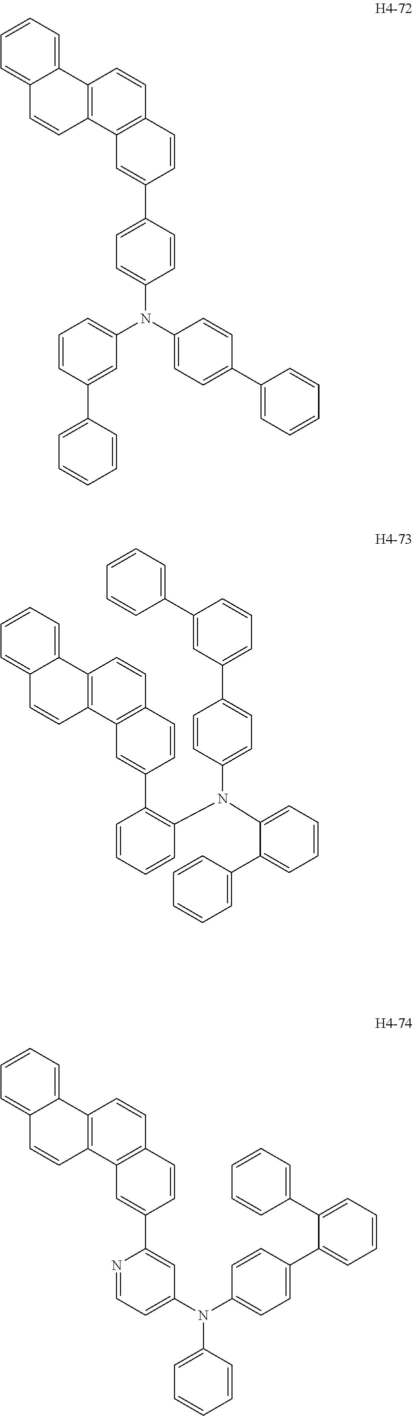 [SMILES: c1ccc(-c2ccc(N(c3ccc(-c4ccc5ccc6c7ccccc7ccc6c5c4)cc3)c3cccc(-c4ccccc4)c3)cc2)cc1 ; c1ccc(-c2cccc(-c3ccc(N(c4ccccc4-c4ccccc4)c4ccccc4-c4ccc5ccc6c7ccccc7ccc6c5c4)cc3)c2)cc1 ; c1ccc(-c2ccccc2-c2ccc(N(c3ccccc3)c3ccnc(-c4ccc5ccc6c7ccccc7ccc6c5c4)c3)cc2)cc1]

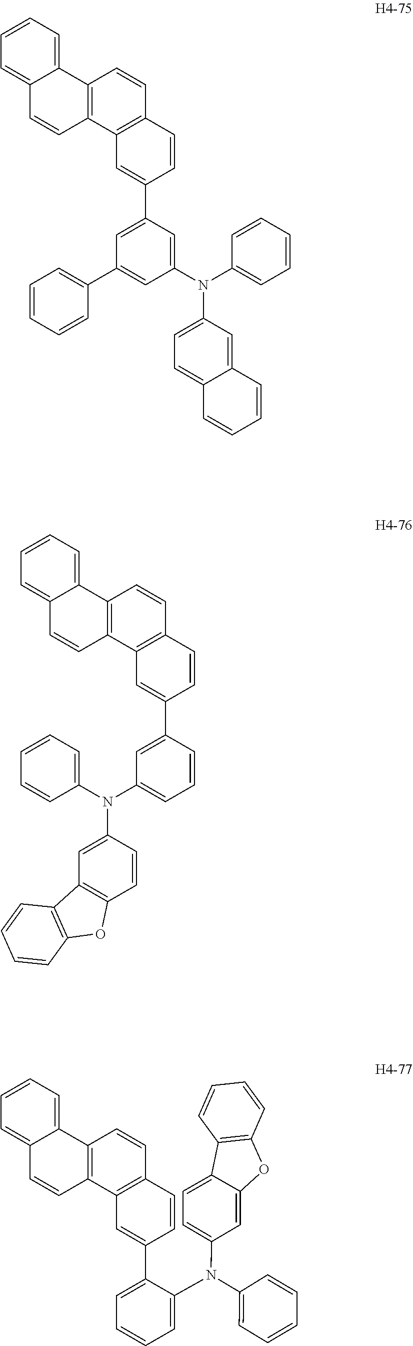 [SMILES: c1ccc(-c2cc(-c3ccc4ccc5c6ccccc6ccc5c4c3)cc(N(c3ccccc3)c3ccc4ccccc4c3)c2)cc1 ; c1ccc(N(c2cccc(-c3ccc4ccc5c6ccccc6ccc5c4c3)c2)c2ccc3oc4ccccc4c3c2)cc1 ; c1ccc(N(c2ccc3c(c2)oc2ccccc23)c2ccccc2-c2ccc3ccc4c5ccccc5ccc4c3c2)cc1]

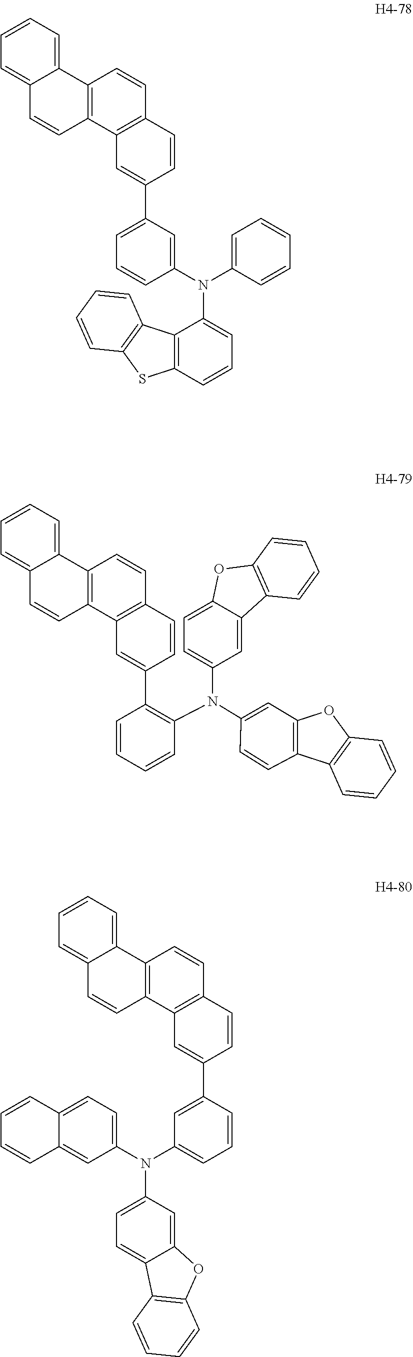 [SMILES: c1ccc(N(c2cccc(-c3ccc4ccc5c6ccccc6ccc5c4c3)c2)c2cccc3sc4ccccc4c23)cc1 ; c1ccc(N(c2ccc3c(c2)oc2ccccc23)c2ccc3oc4ccccc4c3c2)c(-c2ccc3ccc4c5ccccc5ccc4c3c2)c1 ; c1cc(-c2ccc3ccc4c5ccccc5ccc4c3c2)cc(N(c2ccc3ccccc3c2)c2ccc3c(c2)oc2ccccc23)c1]

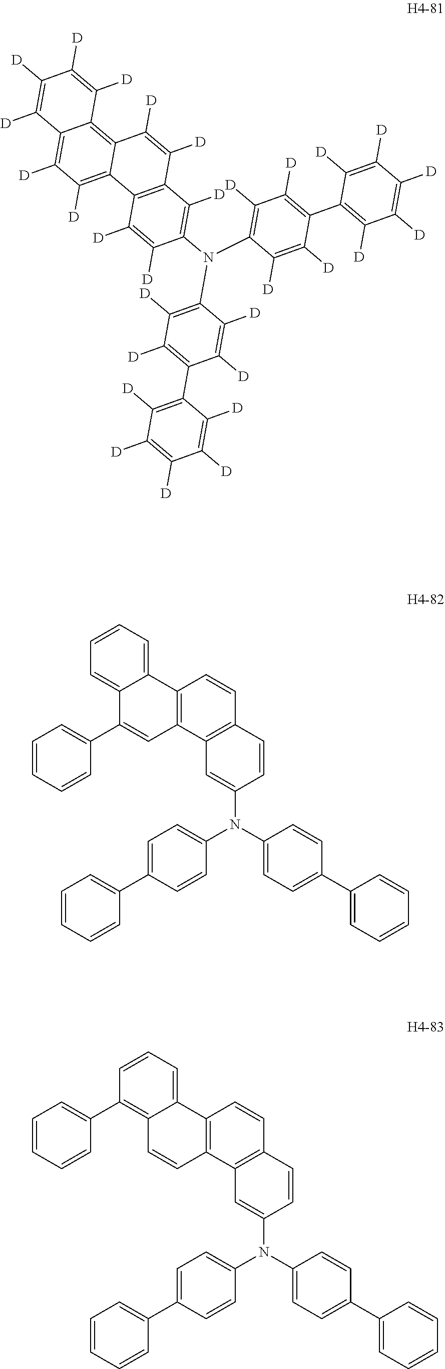 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c5c([2H])c([2H])c([2H])c([2H])c5c([2H])c([2H])c43)c([2H])c2[2H])c([2H])c1[2H] ; c1ccc(-c2ccc(N(c3ccc(-c4ccccc4)cc3)c3ccc4ccc5c6ccccc6c(-c6ccccc6)cc5c4c3)cc2)cc1 ; c1ccc(-c2ccc(N(c3ccc(-c4ccccc4)cc3)c3ccc4ccc5c6cccc(-c7ccccc7)c6ccc5c4c3)cc2)cc1]

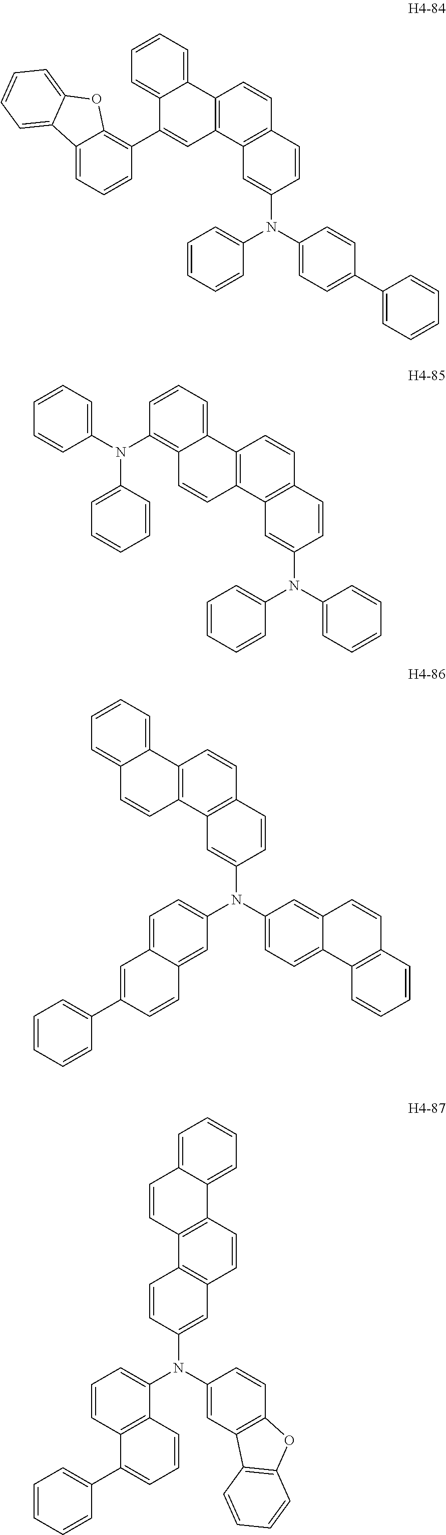 [SMILES: c1ccc(-c2ccc(N(c3ccccc3)c3ccc4ccc5c6ccccc6c(-c6cccc7c6oc6ccccc67)cc5c4c3)cc2)cc1 ; c1ccc(N(c2ccccc2)c2ccc3ccc4c5cccc(N(c6ccccc6)c6ccccc6)c5ccc4c3c2)cc1 ; c1ccc(-c2ccc3cc(N(c4ccc5c(ccc6ccccc65)c4)c4ccc5ccc6c7ccccc7ccc6c5c4)ccc3c2)cc1 ; c1ccc(-c2cccc3c(N(c4ccc5c(ccc6c7ccccc7ccc56)c4)c4ccc5oc6ccccc6c5c4)cccc23)cc1]

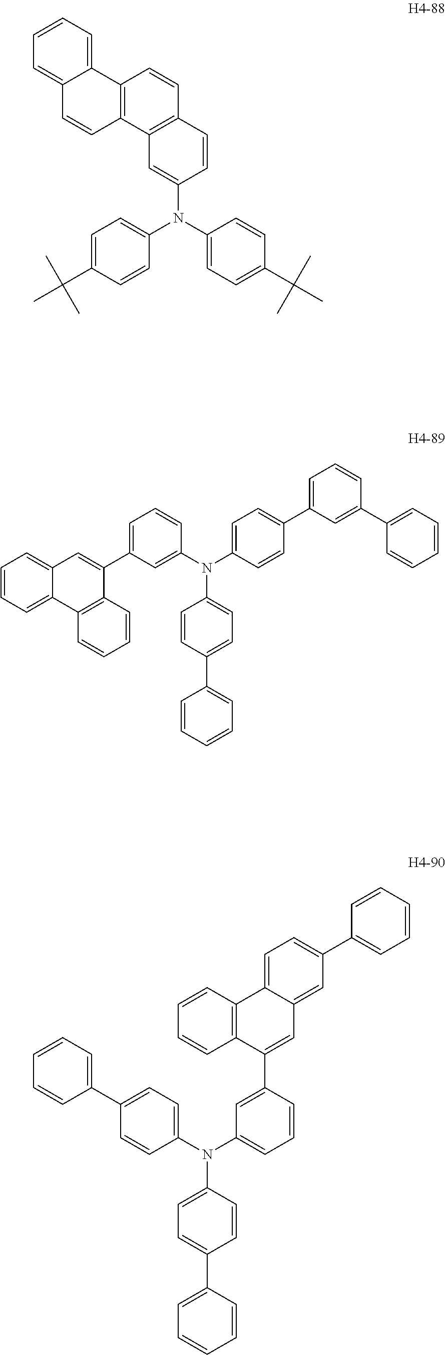 [SMILES: CC(C)(C)c1ccc(N(c2ccc(C(C)(C)C)cc2)c2ccc3ccc4c5ccccc5ccc4c3c2)cc1 ; c1ccc(-c2ccc(N(c3ccc(-c4cccc(-c5ccccc5)c4)cc3)c3cccc(-c4cc5ccccc5c5ccccc45)c3)cc2)cc1 ; c1ccc(-c2ccc(N(c3ccc(-c4ccccc4)cc3)c3cccc(-c4cc5cc(-c6ccccc6)ccc5c5ccccc45)c3)cc2)cc1]

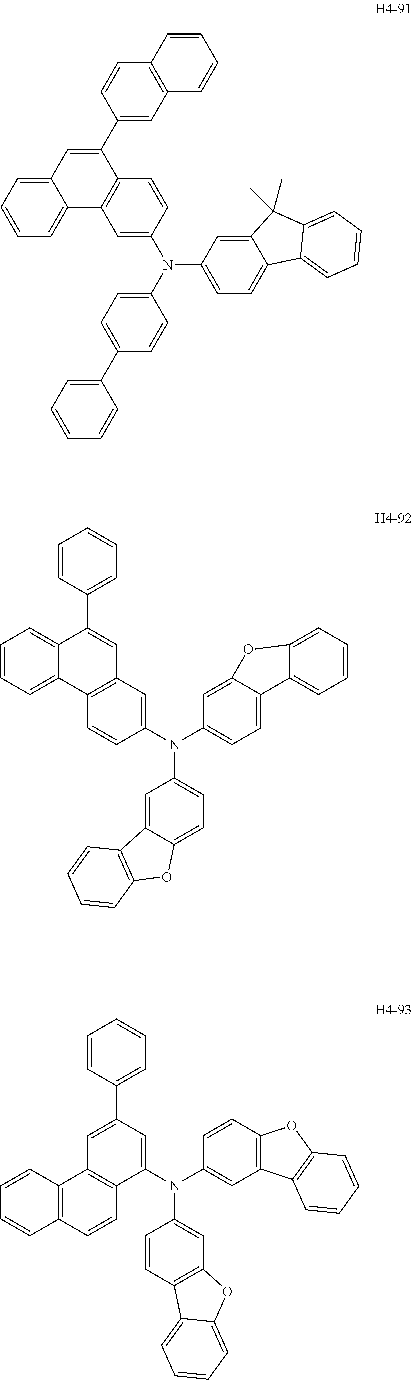 [SMILES: CC1(C)c2ccccc2-c2ccc(N(c3ccc(-c4ccccc4)cc3)c3ccc4c(-c5ccc6ccccc6c5)cc5ccccc5c4c3)cc21 ; c1ccc(-c2cc3cc(N(c4ccc5c(c4)oc4ccccc45)c4ccc5oc6ccccc6c5c4)ccc3c3ccccc23)cc1 ; c1ccc(-c2cc(N(c3ccc4c(c3)oc3ccccc34)c3ccc4oc5ccccc5c4c3)c3ccc4ccccc4c3c2)cc1]

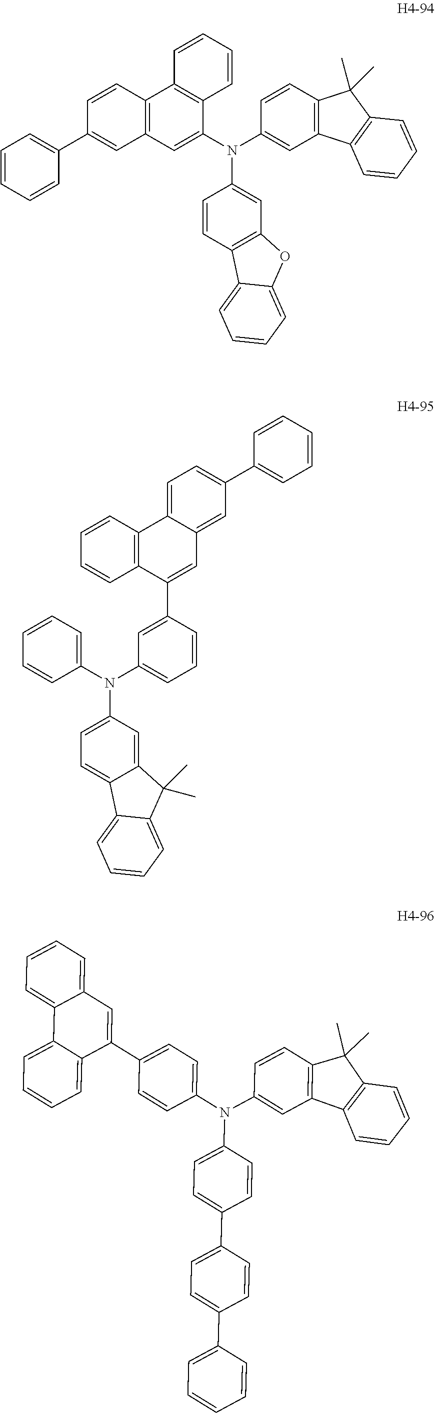 [SMILES: CC1(C)c2ccccc2-c2cc(N(c3ccc4c(c3)oc3ccccc34)c3cc4cc(-c5ccccc5)ccc4c4ccccc34)ccc21 ; CC1(C)c2ccccc2-c2ccc(N(c3ccccc3)c3cccc(-c4cc5cc(-c6ccccc6)ccc5c5ccccc45)c3)cc21 ; CC1(C)c2ccccc2-c2cc(N(c3ccc(-c4ccc(-c5ccccc5)cc4)cc3)c3ccc(-c4cc5ccccc5c5ccccc45)cc3)ccc21]

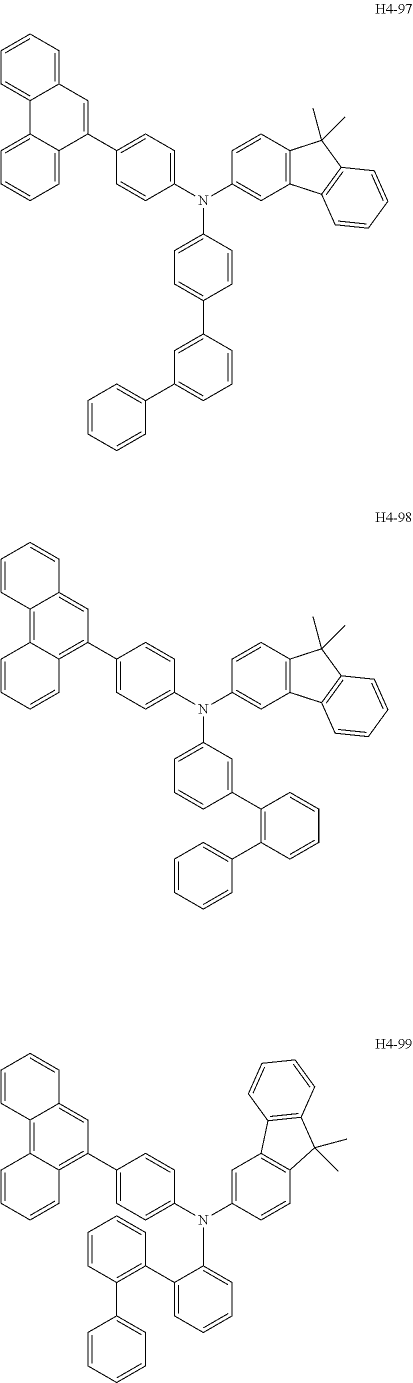 [SMILES: CC1(C)c2ccccc2-c2cc(N(c3ccc(-c4cccc(-c5ccccc5)c4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3cccc(-c4ccccc4-c4ccccc4)c3)ccc21 ; CC1(C)c2ccccc2-c2cc(N(c3ccc(-c4cc5ccccc5c5ccccc45)cc3)c3ccccc3-c3ccccc3-c3ccccc3)ccc21]

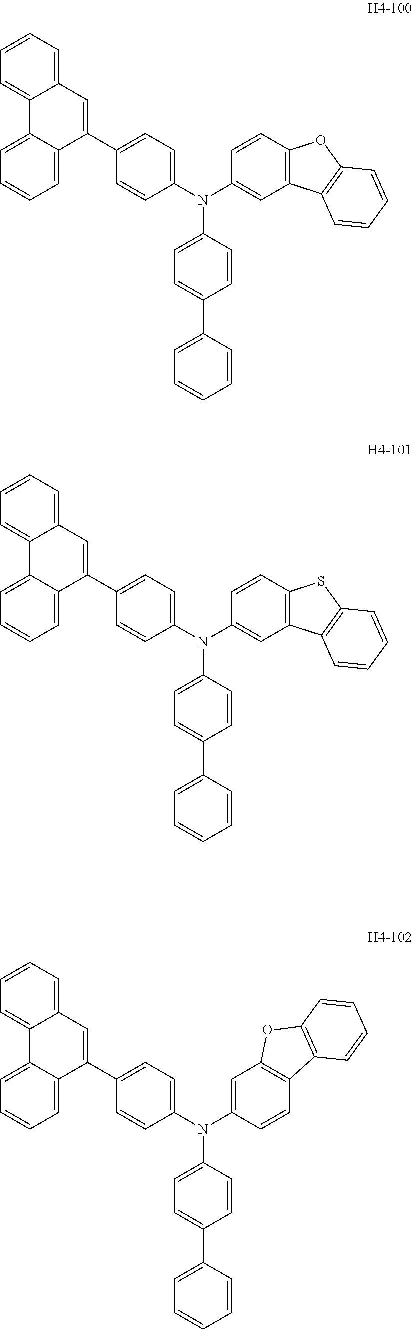 [SMILES: c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4sc5ccccc5c4c3)cc2)cc1 ; c1ccc(-c2ccc(N(c3ccc(-c4cc5ccccc5c5ccccc45)cc3)c3ccc4c(c3)oc3ccccc34)cc2)cc1]

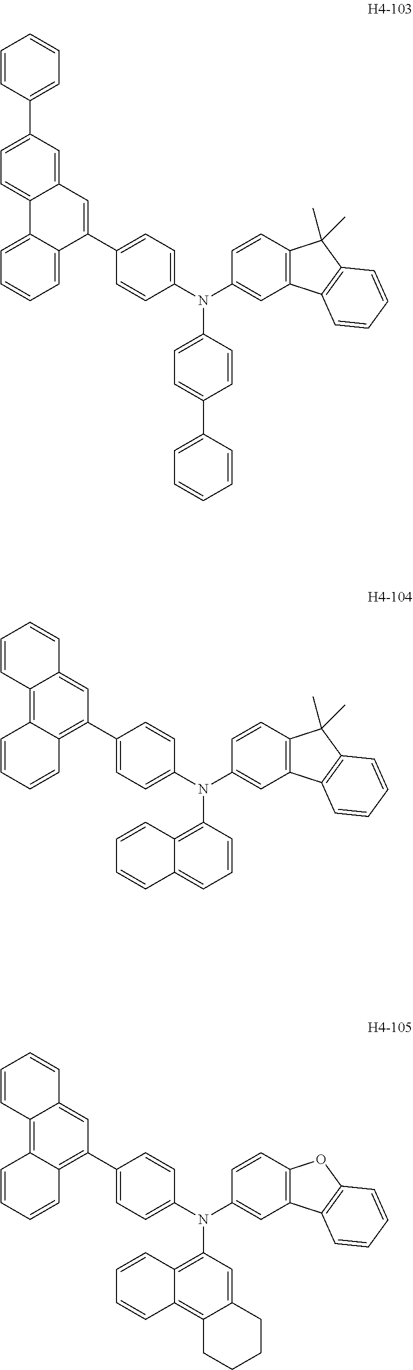 [SMILES: CC1(C)c2ccccc2-c2cc(N(c3ccc(-c4ccccc4)cc3)c3ccc(-c4cc5cc(-c6ccccc6)ccc5c5ccccc45)cc3)ccc21 ; CC1(C)c2ccccc2-c2cc(N(c3ccc(-c4cc5ccccc5c5ccccc45)cc3)c3cccc4ccccc34)ccc21 ; c1ccc2c(c1)cc(-c1ccc(N(c3ccc4oc5ccccc5c4c3)c3cc4c(c5ccccc35)CCCC4)cc1)c1ccccc12]

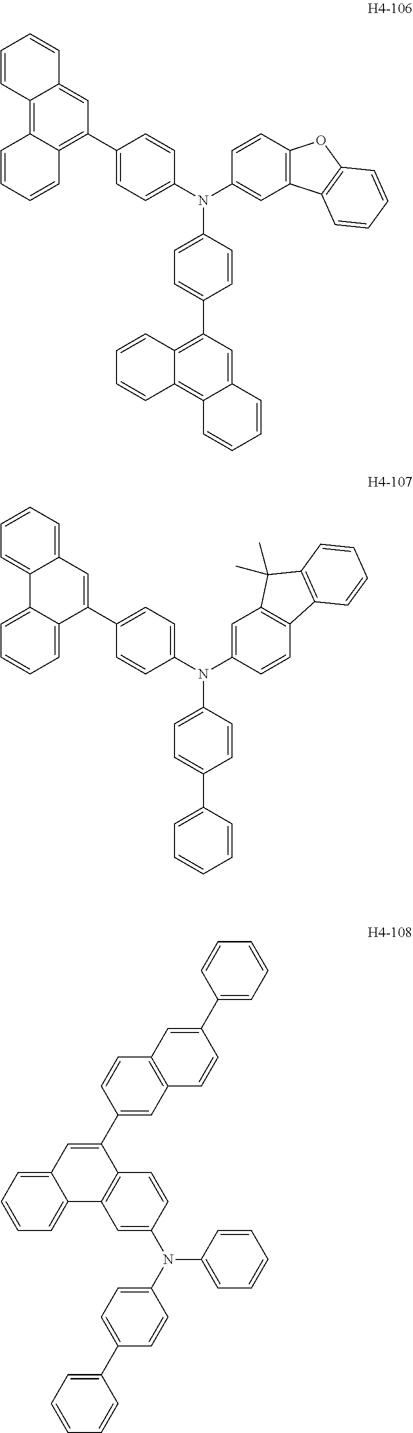 [SMILES: c1ccc2c(c1)cc(-c1ccc(N(c3ccc(-c4cc5ccccc5c5ccccc45)cc3)c3ccc4oc5ccccc5c4c3)cc1)c1ccccc12 ; CC1(C)c2ccccc2-c2ccc(N(c3ccc(-c4ccccc4)cc3)c3ccc(-c4cc5ccccc5c5ccccc45)cc3)cc21 ; c1ccc(-c2ccc(N(c3ccccc3)c3ccc4c(-c5ccc6cc(-c7ccccc7)ccc6c5)cc5ccccc5c4c3)cc2)cc1]

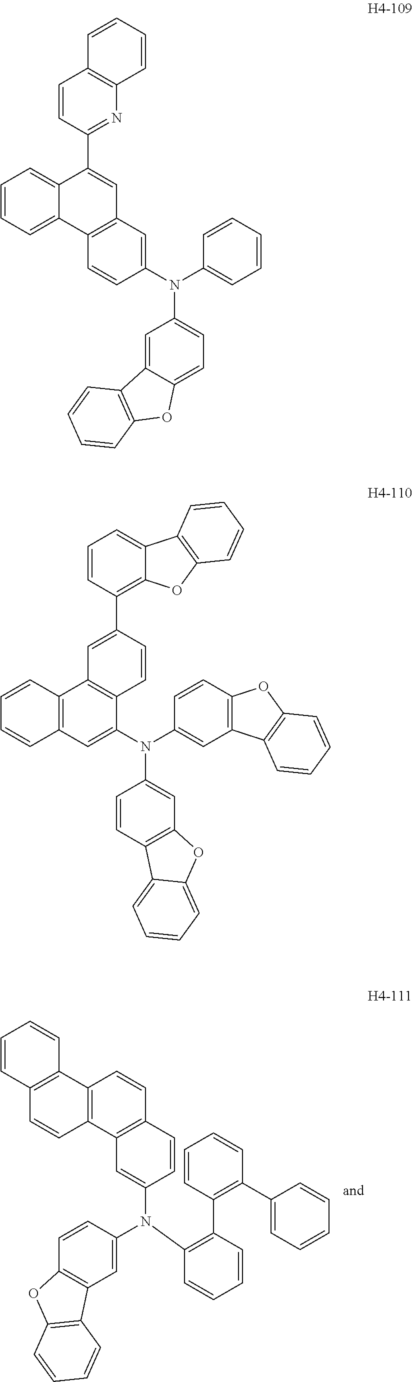 [SMILES: c1ccc(N(c2ccc3c(c2)cc(-c2ccc4ccccc4n2)c2ccccc23)c2ccc3oc4ccccc4c3c2)cc1 ; c1ccc2c(c1)cc(N(c1ccc3c(c1)oc1ccccc13)c1ccc3oc4ccccc4c3c1)c1ccc(-c3cccc4c3oc3ccccc34)cc12 ; c1ccc(-c2ccccc2-c2ccccc2N(c2ccc3ccc4c5ccccc5ccc4c3c2)c2ccc3oc4ccccc4c3c2)cc1]

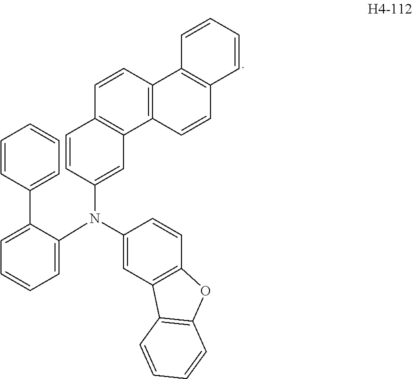 [SMILES: c1ccc(-c2ccccc2N(c2ccc3ccc4c5ccccc5ccc4c3c2)c2ccc3oc4ccccc4c3c2)cc1]

21. An organic electroluminescent device comprising the organic electroluminescent compound according to claim 1.

22. An organic electroluminescent device comprising: a first electrode; a second electrode; and at least one light-emitting layer(s) between the first electrode and the second electrode, wherein the at least one light-emitting layer(s) comprises the plurality of host materials according to claim 8.

23. An organic electroluminescent device comprising: a first electrode; a second electrode; and at least one light-emitting layer(s) between the first electrode and the second electrode, wherein the at least one light-emitting layer(s) comprises the plurality of host materials according to claim 10.

24. An organic electroluminescent device comprising: a first electrode; a second electrode; and at least one light-emitting layer(s) between the first electrode and the second electrode, wherein the at least one light-emitting layer(s) comprises the plurality of host materials according to claim 16.

25. An organic electroluminescent device comprising: a first electrode; a second electrode; and at least one light-emitting layer(s) between the first electrode and the second electrode, wherein the at least one light-emitting layer(s) comprises the plurality of host materials according to claim 18.
